(19) United States
(12) Patent Application Publication
(10) Pub. No.: US 2025/0320226 A1
(43) Pub. Date: Oct. 16, 2025

(54) METHODS OF PREPARING AND CRYSTALLINE FORMS OF (6A,12A)-17-AMINO-12-METHYL-6,15-BIS(TRIFLUOROMETHYL)-13,19-DIOXA-3,4,18-TRIAZATRICYCLO[12.3.1.12,5]NONADECA-1(18),2,4,14,16-PENTAEN-6-OL

(71) Applicant: Vertex Pharmaceuticals Incorporated, Boston, MA (US)

(72) Inventors: Paul Timothy ANGELL, Carlsbad, CA (US); Minson BAEK, San Diego, CA (US); Kevin GAGNON, Acton, MA (US); Daniel James MACK, Boston, MA (US); Ales MEDEK, Winchester, MA (US); Andrey PERESYPKIN, Newton, MA (US); Stefanie ROEPER, Medford, MA (US); Muna SHRESTHA, Belmont, MA (US); David A SIESEL, San Diego, CA (US); Jinglan ZHOU, San Diego, CA (US); Yi SHI, Natick, MA (US)

(21) Appl. No.: 18/835,242

(22) PCT Filed: Feb. 2, 2023
(86) PCT No.: PCT/US2023/012229

§ 371 Date: Aug. 1, 2024

Related U.S. Application Data
(60) Provisional application No. 63/306,443, filed on Feb. 3, 2022.
(60) Provisional application No. 63/308,456, filed on Feb. 9, 2022.

(51) Int. Cl. C07D 498/18; C07D 413/04; C07D 498/08

(52) U.S. Cl. CPC C07D 498/18; C07D 413/04; C07D 498/08

(57) ABSTRACT

Processes and methods of preparing Compound (I) are disclosed. Crystalline forms of Compound (I), pharmaceutically acceptable salts, solvates, hydrates, and cocrystals thereof, pharmaceutical compositions comprising the same, methods of treating cystic fibrosis using the same, and methods for making the same are also disclosed.

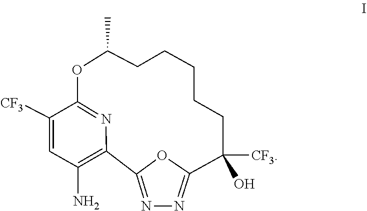 [SMILES: C[C@@H]1CCCCC[C@](O)(C(F)(F)F)C2=NN=C(O2)c2nc(c(C(F)(F)F)cc2N)O1]

This application claims the benefit of U.S. Provisional Application No. 63/306,443, filed on Feb. 3, 2022, and U.S. Provisional Application No. 63/308,456, filed on Feb. 9, 2022, the contents of which are incorporated by reference in their entirety.

Disclosed herein are processes and methods of preparing modulators of cystic fibrosis transmembrane conductance regulator (CFTR) and crystalline and amorphous solid forms of CFTR modulators, pharmaceutical compositions thereof, methods of treating cystic fibrosis with any of the foregoing, and processes for making the crystalline and amorphous forms.

Cystic fibrosis (CF) is a recessive genetic disease that affects approximately 88,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S. et al. (1990) Proc. Natl. Acad. Sci. USA 87:8447-8451). To date, greater than 2000 mutations in the CF gene have been identified; currently, the CFTR2 database contains information on at least 322 of these identified mutations, with sufficient evidence to define at least 281 mutations as disease-causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC (epithelial sodium channel) and CFTR present on the apical membrane and the Na⁺—K⁺-ATPase pump and Cl⁻ channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K-ATPase pump and the basolateral membrane K⁺ channels on the basolateral surface and CFTR on the luminal side coordinate the secretion of chloride. Because water is probably never actively transported itself, its flow across epithelia depends on tiny transepithelial osmotic gradients generated by the bulk flow of sodium and chloride.

A number of CFTR modulators have recently been identified. These modulators can be characterized as, for example, potentiators, correctors, potentiator enhancers/co-potentiators, amplifiers, readthrough agents, and nucleic acid therapies. CFTR modulators that increase the channel gating activity of mutant and wild-type CFTR at the epithelial cell surface are known as potentiators. Correctors improve faulty protein processing and resulting trafficking to the epithelial surface. Ghelani and Schneider-Futschik (2020) ACS Pharmacol. Transl. Sci. 3:4-10. There are three CFTR correctors approved by the U.S. FDA for treatment of cystic fibrosis. However, monotherapy with some CFTR correctors has not been found to be effective enough and as a result combination therapy with a potentiator is needed to enhance CFTR activity. There is currently only one CFTR potentiator that is approved for the treatment of cystic fibrosis. Thus, although the treatment of cystic fibrosis has been transformed by these new small molecule CFTR modulators, new and better modulators are needed to prevent disease progression, reduce the severity of the cystic fibrosis and other CFTR-mediated diseases, and to treat the more severe forms of these diseases.

The compound, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol (Compound I), is a modulator of CFTR activity and thus useful in treating CFTR-mediated diseases such as CF. Compound I has the following structure:

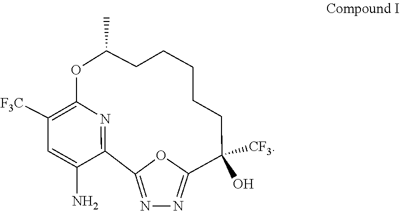 [SMILES: C[C@@H]1CCCCC[C@](O)(C(F)(F)F)C2=NN=C(O2)c2nc(c(C(F)(F)F)cc2N)O1]

Compound I is disclosed in PCT International Application No. PCT/US2021/044895, which published as WO 2022/032068, and which is incorporated herein by reference in its entirety. There remains, however, a need for efficient processes for the synthesis of Compound I that delivers this compound, or pharmaceutically acceptable salts thereof, for example, in higher yield, with higher selectivity, or with higher purity relative to known processes.

Thus, one aspect of the disclosure provides methods of preparing Compound I, stereoisomers of Compound I, deuterated derivatives of Compound I and its stereoisomers, and pharmaceutically acceptable salts of any of the foregoing.

A further aspect of the disclosure provides solid forms of Compound I and pharmaceutically acceptable salts thereof. Compound I was first described in WO 2022/032068 as a heptane solvate.

Crystalline forms are of interest in the pharmaceutical industry, where the control of the crystalline form(s) of the active ingredient may be desirable or even required. Reproducible processes for producing a compound with a particular crystalline form in high purity may be desirable for compounds intended to be used in pharmaceuticals, as different crystalline forms may possess different properties. For example, different crystalline forms may possess different chemical, physical, and/or pharmaceutical properties. In some embodiments, one or more crystalline forms disclosed herein may exhibit a higher level of purity, chemical stability, and/or physical stability compared to the forms produced in WO 2022/032068. Certain crystalline forms (e.g., crystalline free form, crystalline salt, crystalline salt solvate, and crystalline salt hydrate forms of Compound I (collectively referred to as “crystalline forms”)) may exhibit lower hygroscopicity than the forms produced in WO 2022/032068. Thus, the crystalline forms of this disclosure may provide advantages during drug substance manufacturing, storage, and handling over the amorphous forms produced in WO 2022/032068. Thus, pharmaceutically acceptable crystalline forms of Compound I may be particularly useful for the production of drugs for the treatment of CFTR-mediated diseases.

In some embodiments, the crystalline form of Compound I is Compound I neat Form A. In some embodiments, the crystalline form of Compound I is Compound I neat Form B. In some embodiments, the crystalline form of Compound I is Compound I hemihydrate Form C. In some embodiments, the crystalline form of Compound I is Compound I neat Form D. In some embodiments, the crystalline form of Compound I is Compound I neat Form E. In some embodiments, the crystalline form of Compound I is Compound I acetic acid solvate. In some embodiments, the crystalline form of Compound I is Compound I heptane solvate Form B. In some embodiments, the crystalline form of Compound I is Compound I heptane solvate Form C. In some embodiments, the crystalline form of Compound I is Compound I octane solvate. In some embodiments, the crystalline form of Compound I is Compound I cyclohexane solvate Form A. In some embodiments, the crystalline form of Compound I is Compound I cyclohexane solvate Form B. In some embodiments, the crystalline form of Compound I is Compound I cyclohexane solvate Form C. In some embodiments, the crystalline form of Compound I is Compound I ethanol solvate. In some embodiments, the crystalline form of Compound I is Compound I solvate/hydrate (dry). In some embodiments, the crystalline form of Compound I is Compound I solvate/hydrate (wet). In some embodiments, the crystalline form of Compound I is Compound I L-lysine cocrystal. In some embodiments, the crystalline form of Compound I is Compound I L-arginine cocrystal. In some embodiments, the crystalline form of Compound I is Compound I L-phenylalanine cocrystal. In some embodiments, the crystalline form of Compound I is Compound I succinic acid cocrystal (wet). In some embodiments, the crystalline form of Compound I is Compound I succinic acid cocrystal (dry). In some embodiments, the crystalline form of Compound I is Compound I methanol solvate/hydrate.

In some embodiments, the solid form of Compound I is an amorphous form. In some embodiments, the solid amorphous form of Compound I is Compound I neat amorphous form.

Another aspect of the invention provides pharmaceutical compositions comprising at least one solid form chosen from solid forms of Compound I, pharmaceutically acceptable salts thereof, and deuterated derivives of any of the foregoing disclosed herein, which compositions may further include at least one additional active pharmaceutical ingredient and/or at least one carrier.

In certain embodiments, the pharmaceutical compositions of the invention comprise Compound I in any of the pharmaceutically acceptable solid forms disclosed herein. In some embodiments, compositions comprising Compound I in any of the pharmaceutically acceptable crystalline forms disclosed herein may optionally further comprise at least one compound chosen from Compound II, Compound III, Compound III-d, Compound IV, Compound V, Compound VI, Compound VII, Compound VIII, Compound IX, Compound X, and pharmaceutically acceptable salts and deuterated derivatives thereof.

Another aspect of the invention provides methods of treating the CFTR-mediated disease cystic fibrosis comprising administering Compound I in any of the pharmaceutically acceptable solid forms disclosed herein, optionally as part of a pharmaceutical composition comprising at least one additional component (such as a carrier or additional active agent), to a subject in need thereof. In some embodiments, methods of treating the CFTR-mediated disease cystic fibrosis comprise administering Compound I in any of the pharmaceutically acceptable solid forms disclosed herein, and optionally further administering one or more additional CFTR modulating agents selected from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II), N-[2,4-bis(1,1-dimethylethyl)-5-hydroxyphenyl]-1,4-dihydro-4-oxoquinoline-3-carboxamide (Compound III) or N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound III-d), 3-(6-(1-(2,2-difluorobenzo[d][1,3]dioxol-5-yl)cyclopropane carboxamido)-3-methylpyridin-2-yl)benzoic acid (Compound IV), N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound V), N-(benzenesulfonyl)-6-[3-[2-[1-(trifluoromethyl) cyclopropyl]ethoxy]pyrazol-1-yl]-2-[(4S)-2,2,4-trimethylpyrrolidin-1-yl]pyridine-3-carboxamide (Compound VI), (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione (Compound VII), (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione (Compound VIII); N-(benzenesulfonyl)-6-(3-fluoro-5-isobutoxy-phenyl)-2-[(4S)-2,2,4-trimethylpyrrolidin-1-yl]pyridine-3-carboxamide (Compound IX), and N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound X).

A further aspect of the disclosure provides processes of making the solid forms of Compound I disclosed herein.

Another aspect of the invention provides solid forms of Compound I, pharmaceutically acceptable salts thereof, and deuterated derivives of any of the foregoing disclosed herein, for use in any of the methods described herein.

### BRIEF DESCRIPTION OF THE FIGURES

FIG. 1 provides an X-ray power diffraction (XRPD) pattern of Compound I neat amorphous form.

FIG. 2 provides a thermogravimetric analysis (TGA) curve for Compound I neat amorphous form.

FIG. 3 provides a differential scanning calorimetry (DSC) analysis of Compound I neat amorphous form.

FIG. 4 provides a ¹³C solid-state NMR (SSNMR) spectrum of Compound I neat amorphous form.

FIG. 5 provides a ¹⁹F SSNMR spectrum of Compound I neat amorphous form.

FIG. 6 provides an XRPD pattern of crystalline Compound I neat Form A.

FIG. 7 provides a TGA curve for crystalline Compound I neat Form A.

FIG. 8 provides a DSC analysis of crystalline Compound I neat Form A.

FIG. 9 provides an XRPD pattern of crystalline Compound I neat Form B.

FIG. 10 provides a TGA curve for crystalline Compound I neat Form B.

FIG. 11 provides a DSC analysis of crystalline Compound I neat Form B.

FIG. 12 provides a ¹³C SSNMR spectrum of crystalline Compound I neat Form B.

FIG. 13 provides a ¹⁹F SSNMR spectrum of crystalline Compound I neat Form B.

FIG. 14 provides an XRPD pattern of crystalline Compound I hemihydrate Form C.

FIG. 15 provides a TGA curve for crystalline Compound I hemihydrate Form C.

FIG. 16 provides a DSC analysis of crystalline Compound I hemihydrate Form C.

FIG. 17 provides a ¹³C SSNMR spectrum of crystalline Compound I hemihydrate Form C.

FIG. 18 provides a ¹⁹F SSNMR spectrum of crystalline Compound I hemihydrate Form C.

FIG. 19 provides an XRPD pattern of crystalline Compound I neat Form D.

FIG. 20 provides a TGA curve for crystalline Compound I neat Form D.

FIG. 21 provides a DSC analysis of crystalline Compound I neat Form D.

FIG. 22 provides a ¹³C SSNMR spectrum of crystalline Compound I neat Form D.

FIG. 23 provides a ¹⁹F SSNMR spectrum of crystalline Compound I neat Form D.

FIG. 24 provides a DSC analysis of crystalline Compound I neat Form E.

FIG. 25 provides an XRPD pattern of crystalline Compound I acetic acid solvate.

FIG. 26 provides a DSC analysis of crystalline Compound I acetic acid solvate.

FIG. 27 provides an XRPD pattern of crystalline Compound I heptane solvate Form B.

FIG. 28 provides a DSC analysis of crystalline Compound I heptane solvate Form B.

FIG. 29 provides a ¹³C SSNMR spectrum of crystalline Compound I heptane solvate Form B.

FIG. 30 provides a ¹⁹F SSNMR spectrum of crystalline Compound I heptane solvate Form B.

FIG. 31 provides an XRPD pattern of crystalline Compound I heptane solvate Form C.

FIG. 32 provides a TGA curve for crystalline Compound I heptane solvate Form C.

FIG. 33 provides a DSC analysis of crystalline Compound I heptane solvate Form C.

FIG. 34 provides a ¹³C SSNMR spectrum of crystalline Compound I heptane solvate Form C.

FIG. 35 provides an XRPD pattern of crystalline Compound I octane solvate.

FIG. 36 provides a ¹³C SSNMR spectrum of crystalline Compound I octane solvate.

FIG. 37 provides a ¹⁹F SSNMR spectrum of crystalline Compound I octane solvate.

FIG. 38 provides an XRPD pattern of crystalline Compound I cyclohexane solvate Form A.

FIG. 39 provides a ¹³C SSNMR spectrum of crystalline Compound I cyclohexane solvate Form A.

FIG. 40 provides a ¹⁹F SSNMR spectrum of crystalline Compound I cyclohexane solvate Form A.

FIG. 41 provides an XRPD pattern of crystalline Compound I cyclohexane solvate Form B.

FIG. 42 provides a DSC analysis of crystalline Compound I cyclohexane solvate Form B.

FIG. 43 provides a ¹³C SSNMR spectrum of crystalline Compound I cyclohexane solvate Form B.

FIG. 44 provides a ¹⁹F SSNMR spectrum of crystalline Compound I cyclohexane solvate Form B.

FIG. 45 provides an XRPD pattern of crystalline Compound I cyclohexane solvate Form C.

FIG. 46 provides an XRPD pattern of crystalline Compound I ethanol solvate.

FIG. 47 provides a ¹³C SSNMR spectrum of crystalline Compound I ethanol solvate.

FIG. 48 provides a ¹⁹F SSNMR spectrum of crystalline Compound I ethanol solvate.

FIG. 49 provides an XRPD pattern of crystalline Compound I solvate/hydrate (dry).

FIG. 50 provides a TGA curve for crystalline Compound I solvate/hydrate (dry).

FIG. 51 provides a DSC analysis of crystalline Compound I solvate/hydrate (dry).

FIG. 52 provides an XRPD pattern of crystalline Compound I solvate/hydrate (wet).

FIG. 53 provides a ¹³C SSNMR spectrum of crystalline Compound I solvate/hydrate (wet).

FIG. 54 provides a ¹⁹F SSNMR spectrum of crystalline Compound I solvate/hydrate (wet).

FIG. 55 provides an XRPD pattern of crystalline Compound I L-lysine cocrystal.

FIG. 56 provides a TGA curve for crystalline Compound I L-lysine cocrystal.

FIG. 57 provides a DSC analysis of crystalline Compound I L-lysine cocrystal.

FIG. 58 provides a ¹³C SSNMR spectrum of crystalline Compound I L-lysine cocrystal.

FIG. 59 provides an XRPD pattern of crystalline Compound I L-arginine cocrystal.

FIG. 60 provides a TGA curve for crystalline Compound I L-arginine cocrystal.

FIG. 61 provides a DSC analysis of crystalline Compound I L-arginine cocrystal.

FIG. 62 provides an XRPD pattern of crystalline Compound I L-phenylalanine cocrystal.

FIG. 63 provides a DSC analysis of crystalline Compound I L-phenylalanine cocrystal.

FIG. 64 provides an XRPD pattern of crystalline Compound I succinic acid cocrystal (wet).

FIG. 65 provides an XRPD pattern of crystalline Compound I succinic acid cocrystal (dry).

FIG. 66 provides a DSC analysis of crystalline Compound I succinic acid cocrystal (dry).

FIG. 67 provides an XRPD pattern of crystalline Compound I methanol solvate/hydrate.

FIG. 68 provides a ¹³C SSNMR spectrum of crystalline Compound I methanol solvate/hydrate.

FIG. 69 provides a ¹⁹F SSNMR spectrum of crystalline Compound I methanol solvate/hydrate.

FIG. 70 provides an X-ray power diffraction (XRPD) pattern of crystalline Compound I heptane solvate Form A.

FIG. 71 provides an XRPD patterns of crystalline Compound I heptane solvate Form A prepared under three different drying conditions.

FIG. 72 provides a DSC analysis of crystalline Compound I heptane solvate Form A.

FIG. 73 provides a ¹³C SSNMR spectrum of crystalline Compound I heptane solvate Form A.

FIG. 74 provides a ¹⁹F SSNMR of crystalline Compound I heptane solvate Form A.

FIG. 75 provides a TGA curve for crystalline Compound I heptane solvate Form A (Drying Condition 1).

FIG. 76 provides a TGA curve for crystalline Compound I heptane solvate Form A (Drying Condition 2).

FIG. 77 provides a TGA curve for crystalline Compound I heptane solvate Form A (Drying Condition 3).

### DEFINITIONS

“Compound I” as used throughout this disclosure refers to (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, which can be depicted as having the following structure:

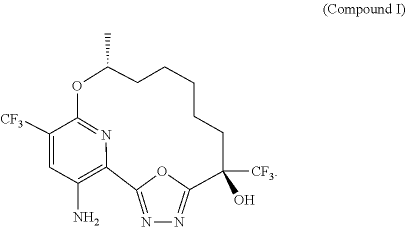 [SMILES: C[C@@H]1CCCCC[C@](O)(C(F)(F)F)C2=NN=C(O2)c2nc(c(C(F)(F)F)cc2N)O1]

Compound I may be a racemic mixture or an enantioenriched (e.g., >90% ee, >95% ee, >98% ee) mixture of isomers. Compound I may be in the form of a pharmaceutically acceptable salt, solvate, and/or hydrate. Compound I and methods for making and using Compound I, stereoisomers of Compound I, deuterated derivatives of Compound I and its stereoisomers, and pharmaceutically acceptable salts of any of the foregoing are disclosed in WO 2022/032068, incorporated herein by reference.

“Compound II” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

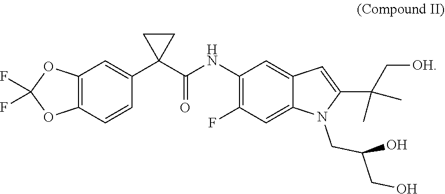 [SMILES: CC(C)(CO)c1cc2cc(NC(=O)C3(c4ccc5c(c4)OC(F)(F)O5)CC3)c(F)cc2n1C[C@@H](O)CO]

Compound II may be in the form of a pharmaceutically acceptable salt. Compound II and methods of making and using Compound II are disclosed in WO 2010/053471, WO 2011/119984, WO 2011/133751, WO 2011/133951, and WO 2015/160787, each incorporated herein by reference. [00100]“Compound III” as used throughout this disclosure refers to N-(5-hydroxy-2,4-di-tert-butyl-phenyl)-4-oxo-1H-quinoline-3-carboxamide which is depicted by the structure:

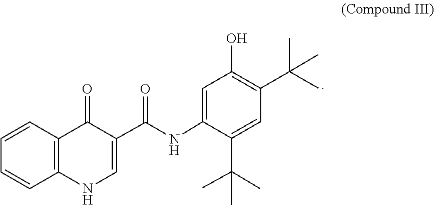 [SMILES: CC(C)(C)c1cc(C(C)(C)C)c(NC(=O)c2c[nH]c3ccccc3c2=O)cc1O]

Compound III may also be in the form of a pharmaceutically acceptable salt. Compound III and methods of making and using Compound III are disclosed in WO 2006/002421, WO 2007/079139, WO 2010/108162, and WO 2010/019239, each incorporated herein by reference.

In some embodiments, a deuterated derivative of Compound III (Compound III-d) is employed in the compositions and methods disclosed herein. A chemical name for Compound III-d is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

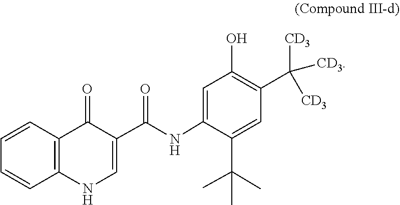 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Compound III-d may be in the form of a pharmaceutically acceptable salt. Compound III-d and methods of making and using Compound III-d are disclosed in WO 2012/158885, WO 2014/078842, and U.S. Pat. No. 8,865,902, incorporated herein by reference.

“Compound IV” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

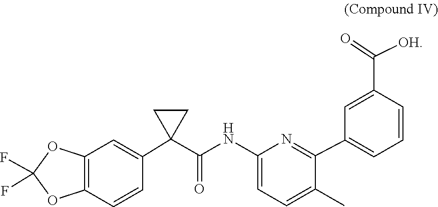 [SMILES: Cc1ccc(NC(=O)C2(c3ccc4c(c3)OC(F)(F)O4)CC2)nc1-c1cccc(C(=O)O)c1]

Compound IV may be in the form of a pharmaceutically acceptable salt. Compound IV and methods of making and using Compound IV are disclosed in WO 2007/056341, WO 2009/073757, and WO 2009/076142, incorporated herein by reference.

“Compound V” as used herein, refers to N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide, which is depicted by the chemical structure:

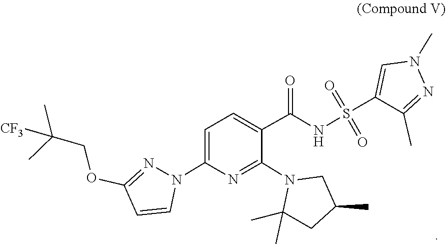 [SMILES: Cc1nn(C)cc1S(=O)(=O)NC(=O)c1ccc(-n2ccc(OCC(C)(C)C(F)(F)F)n2)nc1N1C[C@@H](C)CC1(C)C]

Compound V may be in the form of a pharmaceutically acceptable salt. Compound V and methods of making and using Compound V are disclosed in WO 2018/107100 and WO 2019/113476, incorporated herein by reference.

“Compound VI” as used herein, refers to N-(benzenesulfonyl)-6-[3-[2-[1-(trifluoromethyl) cyclopropyl]ethoxy]pyrazol-1-yl]-2-[(4S)-2,2,4-trimethylpyrrolidin-1-yl]pyridine-3-carboxamide, which is depicted by the chemical structure

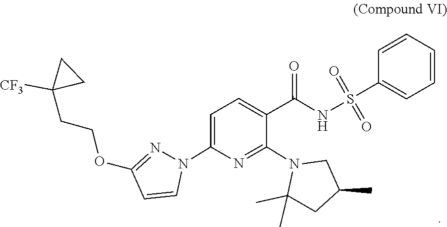 [SMILES: C[C@@H]1CN(c2nc(-n3ccc(OCCC4(C(F)(F)F)CC4)n3)ccc2C(=O)NS(=O)(=O)c2ccccc2)C(C)(C)C1]

Compound VI may be in the form of a pharmaceutically acceptable salt. Compound VI and methods of making and using Compound VI are disclosed in WO 2018/064632, incorporated herein by reference.

“Compound VII” as used herein, refers to (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione, which is depicted by the chemical structure:

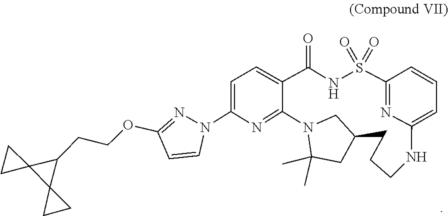 [SMILES: CC1(C)C[C@@H]2CCCNc3cccc(n3)S(=O)(=O)NC(=O)c3ccc(-n4ccc(OCCC5C6(CC6)C56CC6)n4)nc3N1C2]

Compound VII may be in the form of a pharmaceutically acceptable salt. Compound VII and methods of making and using Compound VII are disclosed in WO 2019/161078, WO 2020/102346, and PCT Application No. PCT/US2020/046116, incorporated herein by reference.

“Compound VIII” as used herein, refers to (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione, which is depicted by the chemical structure:

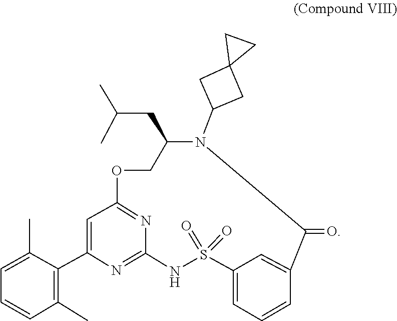 [SMILES: Cc1cccc(C)c1-c1cc2nc(n1)NS(=O)(=O)c1cccc(c1)C(=O)N(C1CC3(CC3)C1)[C@H](CC(C)C)CO2]

Compound VIII may be in the form of a pharmaceutically acceptable salt. Compound VIII and methods of making and using Compound VIII are disclosed in WO 2020/206080, incorporated herein by reference.

“Compound IX” as used herein, refers to N-(benzenesulfonyl)-6-(3-fluoro-5-isobutoxy-phenyl)-2-[(4S)-2,2,4-trimethylpyrrolidin-1-yl]pyridine-3-carboxamide, which is depicted by the chemical structure:

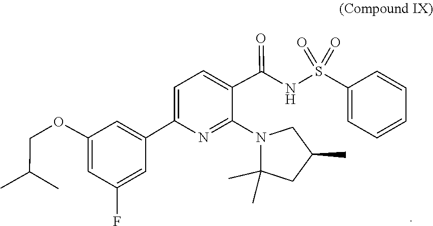 [SMILES: CC(C)COc1cc(F)cc(-c2ccc(C(=O)NS(=O)(=O)c3ccccc3)c(N3C[C@@H](C)CC3(C)C)n2)c1]

Compound IX may be in the form of a pharmaceutically acceptable salt. Compound IX and methods of making and using Compound IX are disclosed in WO 2016/057572, incorporated herein by reference.

“Compound X” as used herein, refers to N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide, which is depicted by the chemical structure:

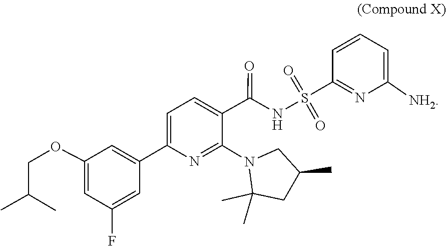 [SMILES: CC(C)COc1cc(F)cc(-c2ccc(C(=O)NS(=O)(=O)c3cccc(N)n3)c(N3C[C@@H](C)CC3(C)C)n2)c1]

Compound X may be in the form of a pharmaceutically acceptable salt. Compound X and methods of making and using Compound X are disclosed in WO 2016/057572, incorporated herein by reference.

As used herein, “CFTR” means cystic fibrosis transmembrane conductance regulator.

As used herein, the terms “CFTR modulator” and “CFTR modulating compound” interchangeably refer to a compound that directly or indirectly increases the activity of CFTR. The increase in activity resulting from a CFTR modulator includes but is not limited to compounds that correct, potentiate, stabilize, and/or amplify CFTR.

As used herein, the term “CFTR corrector” refers to a compound that facilitates the processing and trafficking of CFTR to increase the amount of CFTR at the cell surface.

As used herein, the term “CFTR potentiator” refers to a compound that increases the channel activity of CFTR protein located at the cell surface, resulting in enhanced ion transport.

As used herein, the term “CFTR potentiator enhancer,” “CFTR potentiation enhancer,” and “CFTR co-potentiator” are used interchangeably and refer to a compound that enhances CFTR potentiation.

As used herein, the term “active pharmaceutical ingredient” (“API”) or “therapeutic agent” refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement of CF or one or more of its symptoms or lessening the severity of CF or one or more of its symptoms in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrently with, or subsequent to each other.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. In general, a genetic defect or mutation, or a change in the nucleotides in a gene, results in a mutation in the CFTR protein translated from that gene, or a frame shift(s).

As used herein, the term “F508del” refers to a mutant CFTR protein which is lacking the amino acid phenylalanine at position 508, or to a mutant CFTR gene which encodes for a CFTR protein lacking the amino acid phenylalanine at position 508.

As used herein, the term “alkyl” means a saturated or partially saturated, branched, or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms) in which one or more adjacent carbon atoms is interrupted by a double (alkenyl) or triple (alkynyl) bond. Alkyl groups may be substituted or unsubstituted.

As used herein, the term “unsaturated” means that a moiety has one or more units of unsaturation.

As used herein, the term “pi bond” means a covalent bond formed by the p orbitals of adjacent atoms. Pi bonds exist where there is a multiple bond, i.e., a double or triple bond, between two atoms. For example, a carbon-carbon double bond consists of one pi bond, and a carbon-carbon triple bond consists of two pi bonds.

The term “aliphatic” or “aliphatic group,” as used herein, means a straight-chain (i.e., unbranched) or branched, substituted, or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic,” “carbocycle,” or “cycloalkyl”), that has a single point of attachment to the rest of the molecule. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. In some embodiments, “cycloaliphatic” (or “carbocycle” or “cycloalkyl”) refers to a monocyclic C₃₋₈ hydrocarbon or bicyclic or tricyclic C₈₋₁₄ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof, such as (cycloalkyl)alkyl, (cycloalkenyl)alkyl, and (cycloalkyl)alkenyl. Suitable cycloaliphatic groups include cycloalkyl, bicyclic cycloalkyl (e.g., decalin), bridged bicycloalkyl such as norbornyl or [2.2.2]bicyclo-octyl, and bridged tricyclic such as adamantyl.

As used herein, the term “halogen” or “halo” means F, Cl, Br, or I.

As used herein, the term “haloalkyl group” refers to an alkyl group substituted with one or more halogen atoms, e.g., fluoroalkyl, which refers to an alkyl group substituted with one or more fluorine atoms.

As used herein, term “alkoxy” refers to an alkyl or cycloalkyl covalently bonded to an oxygen atom. Alkoxy groups may be substituted or unsubstituted.

As used herein, the terms “haloaliphatic” and “haloalkoxy” mean aliphatic or alkoxy, as the case may be, substituted with one or more halo atoms. Examples of haloaliphatic include —CHF₂, —CH₂F, —CF₃, —CF₂—, and perhaloalkyl, such as —CF₂CF₃.

As used herein, “cycloalkyl group” refers to a cyclic, non-aromatic hydrocarbon group containing 3-12 carbons in a ring (such as, for example, 3-10 carbons). Cycloalkyl groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, spiro[2.2]pentane, and dispiro[2.0.2.1]heptane. Cycloalkyl groups may be substituted or unsubstituted.

The term “heteroatom” means one or more of oxygen, sulfur, nitrogen, phosphorus, or silicon (including any oxidized form of nitrogen, sulfur, phosphorus, or silicon; the quaternized form of any basic nitrogen; and a substitutable nitrogen of a heterocyclic ring, for example, N (as in 3,4-dihydro-2H-pyrrolyl), NH (as in pyrrolidinyl) or NR⁺ (as in N-substituted pyrrolidinyl)).

The term “heteroaliphatic,” as used herein, means aliphatic groups wherein one or two carbon atoms are independently replaced with one or more heteroatoms, for example, oxygen, sulfur, nitrogen, phosphorus, or silicon. Heteroaliphatic groups may be substituted or unsubstituted, branched or unbranched, cyclic or acyclic, and include “heterocycle,” “heterocyclyl,” “heterocycloaliphatic,” and “heterocyclic” groups.

The term “heterocyclyl,” “heterocycle,” “heterocycloaliphatic,” or “heterocyclic” as used herein means non-aromatic monocyclic, bicyclic, tricyclic, polycyclic, bridged, fused, and spiro ring systems, including mono spiro and dispiro ring systems, in which one or more ring members is an independently chosen heteroatom. In some embodiments, the “heterocycle,” “heterocyclyl,” “heterocycloaliphatic,” or “heterocyclic” group has three to fourteen ring members in which one or more ring members is a heteroatom independently chosen from oxygen, sulfur, nitrogen, and phosphorus, and each ring in the system contains three to seven ring members.

As used herein, the term “aryl” is a functional group or substituent derived from an aromatic ring and encompasses monocyclic aromatic rings and bicyclic, tricyclic, and fused ring systems wherein at least one ring in the system is aromatic. An aryl group may be optionally substituted with one or more substituents. Non-limiting examples of aryl groups include phenyl, naphthyl, and 1,2,3,4-tetrahydronaphthalenyl.

As used herein, the term “heteroaryl” refers to an aromatic ring comprising at least one ring atom that is a heteroatom, such as O, N, or S. Heteroaryl groups encompass monocyclic, bicyclic, and tricyclic ring systems having a total of five to fourteen ring members, wherein at least one ring in the system is aromatic, at least one ring in the system contains one or more heteroatoms, and wherein each ring in the system contains three to seven ring members. Non-limiting examples of heteroaryl rings include pyridine, quinoline, indole, and indoline. A heteroaryl group may be optionally substituted with one or more substituents. In certain embodiments, the term “heteroaryl ring” encompasses heteroaryl rings with various oxidation states, such as heteroaryl rings containing N-oxides and sulfoxides. Non-limiting examples of such heteroaryl rings include pyrimidine N-oxides, quinoline N-oxides, thiophene S-oxides, and pyrimidine N-oxides.

The term “stable,” as used herein, refers to compounds that are not substantially altered when subjected to conditions to allow for their production, detection, and preferably their recovery, purification, and use for one or more of the purposes disclosed herein.

The term “chemically stable,” as used herein, means that the solid form of Compound I does not decompose into one or more different chemical compounds when subjected to specified conditions, e.g., 40° C./75% relative humidity, for a specific period of time, e.g., 1 day, 2 days, 3 days, 1 week, 2 weeks, or longer. In some embodiments, less than 25% of the solid form of Compound I decomposes. In some embodiments, less than about 20%, less than about 15%, less than about 10%, less than about 5%, less than about 3%, less than about 1%, less than about 0.5% of the form of Compound I decomposes under the conditions specified. In some embodiments, no detectable amount of the solid form of Compound I decomposes.

The term “physically stable,” as used herein, means that the solid form of Compound I does not change into one or more different physical forms of Compound I (e.g., different solid forms as measured by XRPD, DSC, etc.) when subjected to specific conditions, e.g., 40° C./75% relative humidity, for a specific period of time, e.g, 1 day, 2 days, 3 days, 1 week, 2 weeks, or longer. In some embodiments, less than 25% of the solid form of Compound I changes into one or more different physical forms when subjected to specified conditions. In some embodiments, less than about 20%, less than about 15%, less than about 10%, less than about 5%, less than about 3%, less than about 10%, less than about 0.5% of the solid form of Compound I changes into one or more different physical forms of Compound I when subjected to specified conditions. In some embodiments, no detectable amount of the solid form of Compound I changes into one or more physically different solid forms of Compound I.

“Selected from” and “chosen from” are used interchangeably herein.

As used herein, the term “ambient conditions” means room temperature, open air condition and uncontrolled humidity condition. As used herein, the terms “room temperature” and “ambient temperature” mean 15° C. to 30° C.

As used herein, the term “solvent” refers to any liquid in which the product is at least partially soluble (solubility of product >1 g/L).

Non-limiting examples of suitable solvents that may be used in this disclosure include, for example, water (H₂O), methanol (MeOH), methylene chloride or dichloromethane (DCM; CH₂Cl₂), acetonitrile (MeCN; CH₃CN), N,N-dimethylformamide (DMF), dimethylsulfoxide (DMSO), methyl acetate (MeOAc), ethyl acetate (EtOAc), isopropyl acetate (IPAc), tert-butyl acetate (t-BuOAc), isopropyl alcohol (IPA), tetrahydrofuran (THF), 2-methyl tetrahydrofuran (2-MeTHF), methyl ethyl ketone (MEK), tert-butanol, diethyl ether (Et₂O), methyl tert-butyl ether (MTBE), 1,4-dioxane, and N-methylpyrrolidone (NMP).

The term “protecting group,” as used herein, refers to any chemical group introduced into a molecule by chemical modification of a functional group to obtain chemoselectivity in a subsequent chemical reaction.

Methods of adding (a process generally referred to as “protecting”) and removing (process generally referred to as “deprotecting”) protecting groups are well-known in the art and available, for example, in P. J. Kocienski, Protecting Groups, 3^{rd} edition (Thieme, 2005), and in Greene and Wuts, Protective Groups in Organic Synthesis, 4th edition (John Wiley & Sons, New York, 2007), both of which are hereby incorporated by reference in their entirety.

Non-limiting examples of useful protecting groups for amines that may be used in this disclosure include monovalent protecting groups, for example, t-butyloxycarbonyl (Boc), benzyl (Bn), β-methoxyethoxytrityl (MEM), tetrahydropyranyl (THP), 9-fluorenylmethyloxycarbonyl (Fmoc), benzyloxycarbonyl (Cbz), formyl, acetyl (Ac), trifluoroacetyl (TFA), trityl (Tr), and p-toluenesulfonyl (Ts); and divalent protecting groups, for example, benzylidene, 4,5-diphenyl-3-oxazolin-2-one, N-phthalimide, N-dichlorophthalimide, N-tetrachlorophthalimide, N-4-nitrophthalimide, N-thiodiglycoloyl amine, N-dithiasuccinimide, N-2,3-diphenylmaleimide, N-2,3-dimethylmaleimide, N-2,5-dimethylpyrrole, N-2,5-bis(triisopropylsiloxy)pyrrole (BIPSOP), N-1,1,4,4-tetramethyldisilylazacyclopentane (STABASE), N-1,1,3,3-tetramethyl-1,3-disilaisoindoline (Benzostabase, BSB), N-diphenylsilyldiethylene, N-5-substituted 1,3-dimethyl-1,3,5-triazacyclohexan-2-one, N-5-substituted 1,3-dibenzyl-1,3,5-triazacyclohexan-2-one, 1-substituted 3,5-dinitro-4-pyridone, and 1,3,5-dioxazine.

Non-limiting examples of useful protecting groups for alcohols that may be used in this disclosure include, for example, acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), trimethylsilyl (TMS), triethylsilyl (TES), triisopropylsilyl (TIPS), t-butyldimethylsilyl (TBS), and t-butyldiphenylsilyl (TBDPS).

Non-limiting examples of useful protecting groups for carboxylic acids that may be used in this disclosure include, for example, methyl or ethyl esters, substituted alkyl esters such as 9-fluorenylmethyl, methoxymethyl (MOM), methylthiomethyl (MTM), tetrahydropyranyl (THP), tetrahydrofuranyl, β-methoxyethoxymethyl (MEM), 2-(trimethylsilyl)ethoxymethyl (SEM), benzyloxymethyl (BOM), pivaloyloxymethyl (POM), phenylacetoxymethyl, and cyanomethyl, acetyl (Ac), phenacyl, substituted phenacyl esters, 2,2,2-trichloroethyl, 2-haloethyl, ω-chloroalkyl, 2-(trimethylsilyl)ethyl, 2-methylthioethyl, t-butyl, 3-methyl-3-pentyl, dicyclopropylmethyl, cyclopentyl, cyclohexyl, allyl, methallyl, cinnamyl, phenyl (Ph), silyl esters, benzyl and substituted benzyl esters, 2,6-dialkylphenyl, and pentafluorophenyl (PFP).

Non-limiting examples of amine bases that may be used in this disclosure include, for example, 1,8-diazabicyclo[5.4.0]undec-7-ene (DBU), N-methylmorpholine (NMM), triethylamine (Et₃N; TEA), diisopropylethyl amine (i-Pr₂EtN; DIPEA), pyridine, 2,2,6,6-tetramethylpiperidine, 1,5,7-triazabicyclo[4.4.0]dec-5-ene (TBD), 7-methyl-1,5,7-triazabicyclo[4.4.0]dec-5-ene (MTBD), t-Bu-tetramethylguanidine, pyridine, 1,5-diazabicyclo[4.3.0]non-5-ene (DBN), and potassium bis(trimethylsilyl)amide (KHMDS).

Non-limiting examples of carbonate bases that may be used in this disclosure include, for example, sodium carbonate (Na₂CO₃), potassium carbonate (K₂CO₃), cesium carbonate (Cs₂CO₃), lithium carbonate (Li₂CO₃), sodium bicarbonate (NaHCO₃), and potassium bicarbonate (KHCO₃).

Non-limiting examples of alkoxide bases that may be used in this disclosure include, for example, t-AmOLi (lithium t-amylate), t-AmONa (sodium t-amylate), t-AmOK (potassium t-amylate), sodium tert-butoxide (NaOtBu), potassium tert-butoxide (KOtBu), and sodium methoxide (NaOMe; NaOCH₃).

Non-limiting examples of hydroxide bases that may be used in this disclosure include, for example, lithium hydroxide (LiOH), sodium hydroxide (NaOH), and potassium hydroxide.

Non-limiting examples of phosphate bases that may be used in this disclosure include, for example, sodium phosphate tribasic (Na₃PO₄), potassium phosphate tribasic (K₃PO₄), potassium phosphate dibasic (K₂HPO₄), and potassium phosphate monobasic (KH₂PO₄).

Non-limiting examples of acids that may be used in this disclosure include, for example, trifluoroacetic acid (TFA), hydrochloric acid (HCl), methanesulfonic acid (MsOH), phosphoric acid (H₃PO₄), and sulfuric acid (H₂SO₄).

As used herein, the terms “reductant” and “reducing agent” are used interchangeably. Non-limiting examples of reducing agents and reducing conditions that may be used in this disclosure include, for example, H₂ and palladium on carbon; H₂ and palladium on alumina; sodium dithionite (Na₂S₂O₄); iron (Fe) and acetic acid (AcOH); and iron (Fe) and ammonium chloride (NH₄Cl).

As used herein, the terms “oxidant” and “oxidizing agent” are used interchangeably. Non-limiting examples of oxidizing agents and oxidizing conditions that may be used in this disclosure include, for example, manganese dioxide (MnO₂); ruthenium(III) chloride (RuCl₃), sodium periodate (NaIO₄), and water (H₂O); and osmium tetroxide (OsO₄) and sodium periodate (NaIO₄).

As used herein, the term “halogenating agent” means a reagent that introduces one or more halogens into a compound by converting certain functional groups into halides. In some embodiments, a halogenating agent converts an alkene or alkyne to a halide. In some embodiments, a halogenating agent converts a hydroxyl group into a halide. Non-limiting examples of halogenating agents that may be used in this disclosure include, for example, bromine (Br₂), iodine (I₂), and pyridinium tribromide.

As used herein, the terms “alkyl halide” and “haloalkane” are used interchangeably. Alkyl halides are compounds in which one or more hydrogen atoms in an unsubstituted or substituted alkane have been replaced by one or more halogen atoms. Non-limiting examples of alkyl halides include, for example, 1-halopropanes and benzyl halides (e.g., benzyl bromide).

As used herein, the term “alkyl triflate” means a compound in which one or more hydrogen atoms in an unsubstituted or substituted alkane have been replaced by one or more triflate groups (e.g, —OSO₂CF₃). Non-limiting examples of alkyl triflates include, for example, 1-propyltriflate, allyl triflate, and benzyl triflate.

As used herein, the term “alkyl tosylate” means a compounds in which one or more hydrogen atoms in an unsubstituted or substituted alkane have been replaced by one or more tosylate groups (e.g., 4-MeC₆H₄SO₂O—). Non-limiting examples of alkyl tosylates include, for example, 1-propyltosylate, allyl tosylate, and benzyl tosylate.

As used herein, the term “sulfonyl chloride” means a compound in which a sulfonyl group (—SO₂—) is singly bonded to a chloride atom (e.g, RSO₂Cl). Non-limiting examples of sulfonyl chlorides include, for example, methanesulfonyl chloride (MeSO₂Cl), trifluoromethanesulfonyl chloride (F₃CSO₂Cl) benzenesulfonyl chloride (PhSO₂Cl), p-toluenesulfonyl chloride (4-MeC₆H₄SO₂Cl or TsCl), 2-nitrobenzylsulfonyl chloride (2-NO₂C₆H₄SO₂Cl or 2-NsCl), and 4-nitrobenzylsulfonyl chloride (4-NO₂C₆H₄SO₂Cl or 4-NsCl).

Non-limiting examples of suitable sulfonate esters —OSO₂R that may be used in this disclosure include, for example, methanesulfonyl (R=Me), trifluoromethanesulfonyl (R=CF₃) benzenesulfonyl (R=Ph), p-toluenesulfonyl (R=4-MeC₆H₄—), 2-nitrobenzylsulfonyl (R=2-NO₂C₆H₄—), and 4-nitrobenzylsulfonyl (R=4-NO₂C₆H₄—).

The term “compound,” when referring to a compound of this disclosure, refers to a collection of molecules having an identical chemical structure, except that there may be isotopic variation among the constituent atoms of the molecules.

Compounds described herein may optionally be substituted with one or more substituents, such as are illustrated generally above, or as exemplified by particular classes, subclasses, and species of the disclosure. It will be appreciated that the phrase “optionally substituted” is used interchangeably with the phrase “substituted or unsubstituted.” In general, the term “substituted,” whether preceded by the term “optionally” or not, indicates that at least one hydrogen of the “substituted” group is replaced with a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position. Combinations of substituents envisioned by this disclosure are preferably those that result in the formation of stable or chemically feasible compounds.

The term “stable compounds,” as used herein, refers to compounds which possess sufficient stability to allow for their manufacture and which maintain the integrity of the compounds for a sufficient period of time to be useful for the purposes detailed herein (e.g., formulation into therapeutic products, intermediates for use in production of therapeutic compounds, isolatable or storable intermediates, and/or treating a disease or condition responsive to therapeutic agents).

As used herein, the term “stereoisomer” refers to both enantiomers and diastereomers.

It will be appreciated that certain compounds of this invention may exist as separate stereoisomers or enantiomers and/or mixtures of those stereoisomers or enantiomers. As used in the chemical structures disclosed herein, a “wedge” ( [symbol] ) or “hash” ( [symbol] ) bond to a stereogenic atom indicates a chiral center of known absolute stereochemistry (i.e. one stereoisomer). As used in the chemical structures disclosed herein, a “wavy” bond ( [symbol] ) to a stereogenic atom indicates a chiral center of unknown absolute stereochemistry (i.e. one stereoisomer). As used in the chemical structures disclosed herein, a “wavy” bond ( [symbol] ) to a double-bonded carbon indicates a mixture of E/Z isomers. As used in the chemical structures disclosed herein, a [symbol] (“straight”) bond to a stereogenic atom indicates where there is a mixture (e.g., a racemate or enrichment). As used herein, two [symbol] (“straight”) bonds to a double-bonded carbon indicates that the double bond possesses the E/Z stereochemistry as drawn. As used in the chemical structures disclosed herein, a

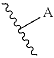

(i.e., a “wavy” line perpendicular to a “straight” bond to group “A”) indicates that group “A” is a substituent whose point of attachment is at the end of the bond that terminates at the “wavy” line.

The terms “about” and “approximately,” when used in connection with doses, amounts, or weight percents of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. As used herein, the terms “about” and “approximately,” when used in connection with amounts, volumes, reaction times, reaction temperatures, etc., in methods or processes, may refer to an acceptable error for a particular value as determined by one of skill in the art, which depends in part on how the values is measured or determined. In some embodiments, the terms “about” and “approximately” mean within 1, 2, 3, or 4 standard deviations. In certain embodiments, the terms “about” and “approximately” mean within 30%, 25%, 20%, 15%, 10%, 9%, 8%, 7%, 6%, 5%, 4%, 3%, 2%, 1%, 0.5%, 0.1%, or 0.05% of a given value or range. In some embodiments, the terms “about” and “approximately” mean within 15%, 10%, 5%, 4%, 3%, 2%, 1%, or 0.5% of a given value or range. In some embodiments, the terms “about” and “approximately” mean within 15% of a given value. In some embodiments, the terms “about” and “approximately” mean within 10% of a given value. As used herein, the symbol “˜” appearing immediately before a numerical value has the same meaning as the terms “about” and “approximately.”

Certain compounds disclosed herein may exist as tautomers and both tautomeric forms are intended, even though only a single tautomeric structure is depicted. For example, a description of Compound A is understood to include its tautomer Compound B and vice versa, as well as mixtures thereof:

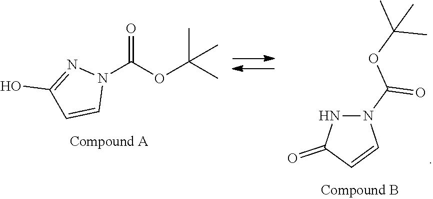

Unless otherwise stated, all tautomeric forms of the compounds of the disclosure are within the scope of the disclosure.

Unless otherwise stated, structures depicted herein are also meant to include all isomeric forms of the structure, e.g., geometric (or conformational), such as (Z) and (E) double bond isomers, and (Z) and (E) conformational isomers. Therefore, geometric and conformational mixtures of the compounds of the disclosure are within the scope of the disclosure.

“Tert” and “t-” are used interchangeably and mean tertiary.

The disclosure also provides processes for preparing salts of the compounds of the disclosure.

A salt of a compound of this disclosure is formed between an acid and a basic group of the compound, such as an amino functional group, or a base and an acidic group of the compound, such as a carboxyl functional group. In some embodiments, the salt is a pharmaceutically acceptable salt.

As used herein, the term “pharmaceutically acceptable salt” means any nontoxic salt that, upon administration to a recipient, is capable of providing, either directly or indirectly, a compound of this disclosure. Pharmaceutically acceptable salts of the compounds of this disclosure include those derived from suitable inorganic and organic acids and bases. A “pharmaceutically acceptable counterion” is an ionic portion of a salt that is not toxic when released from the salt upon administration to a recipient. One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound.

A “free base” form of a compound does not contain an ionically bonded salt. It is noted that the disclosed amounts of the compounds or their pharmaceutically acceptable salts thereof herein are based upon their free base form. For example, “10 mg of at least one compound chosen from Compound I and pharmaceutically acceptable salts thereof” includes 10 mg of Compound I and a concentration of a pharmaceutically acceptable salt of Compound I equivalent to 10 mg of Compound I.

Suitable pharmaceutically acceptable salts are, for example, those disclosed in S. M. Berge, et al. J Pharm. Sci., 1977, 66, 1-19. For example, Table 1 of that article provides the following pharmaceutically acceptable salts:

TABLE 1
Pharmaceutically Acceptable Salts

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | Triethiodide |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |

Non-limiting examples of pharmaceutically acceptable salts derived from appropriate acids include: salts formed with inorganic acids, such as hydrochloric acid, hydrobromic acid, phosphoric acid, sulfuric acid, and perchloric acid; salts formed with organic acids, such as acetic acid, oxalic acid, maleic acid, tartaric acid, citric acid, succinic acid, or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Non-limiting examples of pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions, such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

In some embodiments, the disclosure also is directed to processes for preparing isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope-labelled). Examples of isotopes which are commercially available and suitable for the disclosure include isotopes of hydrogen, carbon, nitrogen, oxygen, phosphorus, fluorine, and chlorine, for example ²H, ³H, ¹³C, ¹⁴C, ¹⁵N, ¹⁸O, ¹⁷O, ³¹P, ³²P, ³⁵S, ¹⁸F, and ³⁶Cl, respectively.

In the compounds of this disclosure, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition.

As used herein, the term “derivative” refers to a collection of molecules having a chemical structure identical to a compound of this disclosure, except that one or more atoms of the molecule may have been substituted with another atom. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C, are within the scope of this disclosure. Such compounds are useful as, for example, analytical tools, probes in biological assays, or compounds with improved therapeutic profiles.

As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group. In chemical structures, deuterium may be represented as “D.”

As used herein, the phrase “deuterated derivatives of [a compound] and its stereoisomers, and pharmaceutically acceptable salts of any of the foregoing” is intended to include deuterated derivatives of the specified compound, deuterated derivatives of any stereoisomers of that compound, and pharmaceutically acceptable salts of the specified compound, pharmaceutically acceptable salts of any of the stereoisomers of that compound, as well as pharmaceutically acceptable salts of deuterated derivatives of the specified compound or its stereoisomers.

In some embodiments, the derivative is a silicon derivative, in which at least one carbon atom in a disclosed compound has been replaced with silicon. In some embodiments, the at least one carbon atom replaced with silicon may be a non-aromatic carbon. In some embodiments, the at least one carbon atom replaced with silicon may be an aromatic carbon. In certain embodiments, the silicon derivatives of the invention may also have one or more hydrogen atoms replaced with deuterium and/or germanium.

In other embodiments, the derivative is a germanium derivative, in which at least one carbon atom in a disclosed compound has been replaced with germanium. In certain embodiments, the germanium derivatives of the invention may also have one or more hydrogen atoms replaced with deuterium and/or silicon.

Because the general properties of silicon and germanium are similar to those of carbon, replacement of carbon by silicon or germanium can result in compounds with similar biological activity to a carbon-containing original compound.

As used herein, the term “pharmaceutically acceptable solid form” refers to a solid form of Compound I of this disclosure wherein the solid form (e.g., crystalline free form, crystalline salt, crystalline salt solvate, crystalline salt hydrate, and amorphous form) of Compound I is nontoxic and suitable for use in pharmaceutical compositions.

As used herein, the term “amorphous” refers to a solid material having no long-range order in the position of its molecules. Amorphous solids are generally supercooled liquids in which the molecules are arranged in a random manner so that there is no well-defined arrangement, e.g., molecular packing, and no long-range order. Amorphous solids are generally isotropic, i.e., exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See US 2004/0006237 for a comparison of XRPDs of an amorphous material and crystalline material. In some embodiments, a solid material may comprise an amorphous compound, and the material may, for example, be characterized by a lack of sharp characteristic crystalline peak(s) in its XRPD spectrum (i.e., the material is not crystalline, but is amorphous, as determined by XRPD). Instead, one or several broad peaks (e.g., halos) may appear in the XRPD pattern of the material. See US 2004/0006237 for a comparison of XRPDs of an amorphous material and crystalline material. A solid material, comprising an amorphous compound, may be characterized by, for example, a wider temperature range for the melting of the solid material, as compared to the range for the melting of a pure crystalline solid. Other techniques, such as, for example, solid state NMR may also be used to characterize crystalline or amorphous forms.

As used herein, the terms “crystal form,” “crystalline form,” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, and ¹³C solid state nuclear magnetic resonance (¹³C SSNMR). Accordingly, as used herein, the terms “crystalline Form [X] of Compound (I)” and “crystalline Form [C] potassium salt of Compound (I)” refer to unique crystalline forms that can be identified and distinguished from each other by one or more characterization techniques including, for example, XRPD, single crystal X-ray diffraction, and ¹³C SSNMR. In some embodiments, the novel crystalline forms are characterized by an X-ray powder diffractogram having one or more signals at one or more specified degree two-theta values (° 2θ).

As used herein, the term “free form” refers to a non-ionized version of the compound in the solid state. Examples of free forms include free bases and free acids.

As used herein, the term “neat form” refers to an unsolvated and unhydrated free form version of a compound in the solid state.

As used herein, the term “solvate” refers to a crystal form comprising one or more molecules of a compound of the present disclosure and, incorporated into the crystal lattice, one or more molecules of a solvent or solvents in stoichiometric or nonstoichiometric amounts. When the solvent is water, the solvate is referred to as a “hydrate.”

In some embodiments, a solid material may comprise a mixture of crystalline solids and amorphous solids. A solid material comprising an amorphous compound may also, for example, contain up to 30% of a crystalline solid. In some embodiments, a solid material prepared to comprise an amorphous compound may also, for example, contain up to 25%, 20%, 15%, 10%, 5%, or 2% of a crystalline solid. In embodiments wherein the solid material contains a mixture of crystalline solids and amorphous solids, the characterizing data, such as XRPD, may contain indicators of both crystalline and amorphous solids. In some embodiments, a crystalline form of this disclosure may contain up to 30% amorphous compound. In some embodiments, a crystalline preparation of Compound I may contain up to 25%, 20%, 15%, 10%, 5%, or 2% of an amorphous solid.

As used herein, the term “substantially amorphous” refers to a solid material having little or no long-range order in the position of its molecules. For example, substantially amorphous materials have less than 15% crystallinity (e.g., less than 10% crystallinity, less than 5% crystallinity, or less than 2% crystallinity). It is also noted that the term “substantially amorphous” includes the descriptor, “amorphous,” which refers to materials having no (0%) crystallinity.

As used herein, the term “substantially crystalline” refers to a solid material having little or no amorphous molecules. For example, substantially crystalline materials have less than 15% amorphous molecules (e.g., less than 10% amorphous molecules, less than 5% amorphous molecules, or less than 2% amorphous molecules). It is also noted that the term “substantially crystalline” includes the descriptor “crystalline,” which refers to materials that are 100% crystalline form.

As used herein, a crystalline form is “substantially pure” when it accounts for an amount by weight equal to or greater than 90% of the sum of all solid form(s) in a sample as determined by a method in accordance with the art, such as quantitative XRPD. In some embodiments, the solid form is “substantially pure” when it accounts for an amount by weight equal to or greater than 95% of the sum of all solid form(s) in a sample. In some embodiments, the solid form is “substantially pure” when it accounts for an amount by weight equal to or greater than 99% of the sum of all solid form(s) in a sample.

As used herein, the terms “X-ray powder diffractogram,” “X-ray powder diffraction pattern,” “XRPD pattern,” “XRPD spectrum” interchangeably refer to an experimentally obtained pattern plotting signal positions (on the abscissa) versus signal intensities (on the ordinate).

A “signal” or “peak” as used herein refers to a point in the XRPD pattern where the intensity as measured in counts is at a local maximum. An XRPD peak is identified by its angular value as measured in degrees 2θ (° 2θ), depicted on the abscissa of an X-ray powder diffractogram, which may be expressed, for example, as “a signal at . . . degrees two-theta,” “a signal at [a] two-theta value(s) of . . . ” and/or “a signal at at least . . . two-theta value(s) selected from . . . .”

The repeatability of the measured angular values is in the range of ±0.2° 2θ, i.e., the angular value can be at the recited angular value +0.2 degrees two-theta, the angular value −0.2 degrees two-theta, or any value between those two end points (angular value +0.2 degrees two-theta and angular value −0.2 degrees two-theta).

One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

As used herein, an X-ray powder diffractogram is “substantially similar to that in [a particular] Figure” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal maximum values in XRPD diffractograms (in degrees two-theta) generally mean that value is identified as ±0.2 degrees two-theta of the reported value, an art-recognized variance.

As used herein, the term “TGA” refers to thermogravimetric analysis and “TGA/DSC” refers to thermogravimetric analysis and differential scnning calorimetry.

As used herein, the term “DSC” refers to the analytical method of differential scanning calorimetry.

As used herein, the term “glass transition temperature” or “Tg” refers to the temperature above which a hard and brittle “glassy” amorphous solid becomes viscous or rubbery.

As used herein, the term “melting temperature”, “melting point”, or “Tm” refers to the temperature at which a material transitions from a solid to a liquid phase.

As used herein, the term “dispersion” refers to a disperse system in which one substance, the dispersed phase, is distributed, in discrete units, throughout a second substance (the continuous phase or vehicle). The size of the dispersed phase can vary considerably (e.g., colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase); or alternatively, an amorphous drug (dispersed phase) in an amorphous polymer (continuous phase). In some embodiments, a solid dispersion includes the polymer constituting the dispersed phase, and the drug constitute the continuous phase. Or, a solid dispersion includes the drug constituting the dispersed phase, and the polymer constituting the continuous phase.

### DETAILED DESCRIPTION OF EMBODIMENTS

Another aspect of the disclosure provides solid forms of Compound I (e.g., crystalline forms, amorphous forms, solvates, hydrates, cocrystals), which can be used in the methods of treatment and pharmaceutical compositions described herein. In some embodiments, the invention provides neat amorphous forms of Compound I. In some embodiments, the invention provides neat crystalline forms of Compound I. In some embodiments, the invention provides solvate crystalline forms of Compound I. In some embodiments, the invention provides hydrate crystalline forms of Compound I. In some embodiments, the invention provides hemihydrate crystalline forms of Compound I. In some embodiments, the invention provides solvate/hydrate crystalline forms of Compound I. In some embodiments, the invention provides cocrystal crystalline forms of Compound I.

### A. Compound I Neat Amorphous Form

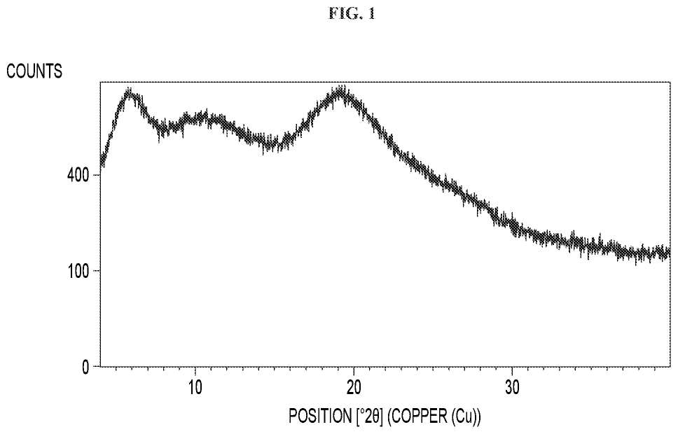

In some embodiments, the invention provides a neat amorphous form of Compound I. In some embodiments, the invention provides Compound I neat amorphous form. FIG. 1 provides an X-ray powder diffractogram of Compound I neat amorphous form at room temperature.

In some embodiments, Compound I neat amorphous form is substantially pure. In some embodiments, Compound I neat amorphous form is substantially amorphous. In some embodiments, Compound I neat amorphous form is characterized by an X-ray powder diffractogram generated by an X-ray powder diffraction analysis with an incident beam of Cu Kα radiation.

In some embodiments, Compound I neat amorphous form is characterized by an X-ray powder diffractogram substantially similar to FIG. 1.

In some embodiments, Compound I neat amorphous form is characterized as having a ¹³C SSNMR spectrum with a peak at 163.8±0.2 ppm. In some embodiments, Compound I neat amorphous form is characterized as having a ¹³C SSNMR spectrum with a peak at 151.9±0.2 ppm. In some embodiments, Compound I neat amorphous form is characterized as having a ¹³C SSNMR spectrum with a peak at 137.6±0.2 ppm. In some embodiments, Compound I neat amorphous form is characterized as having a ¹³C SSNMR spectrum with a peak at 125.8±0.2 ppm. In some embodiments, Compound I neat amorphous form is characterized as having a ¹³C SSNMR spectrum with a peak at 120.8±0.2 ppm. In some embodiments, Compound I neat amorphous form is characterized as having a ¹³C SSNMR spectrum with a peak at 117.8±0.2 ppm. In some embodiments, Compound I neat amorphous form is characterized as having a ¹³C SSNMR spectrum with a peak at 77.3±0.2 ppm. In some embodiments, Compound I neat amorphous form is characterized as having a ¹³C SSNMR spectrum with a peak at 73.6±0.2 ppm. In some embodiments, Compound I neat amorphous form is characterized as having a ¹³C SSNMR spectrum with a peak at 34.5±0.2 ppm. In some embodiments, Compound I neat amorphous form is characterized as having a ¹³C SSNMR spectrum with a peak at 31.4±0.2 ppm. In some embodiments, Compound I neat amorphous form is characterized as having a ¹³C SSNMR spectrum with a peak at 26.3±0.2 ppm. In some embodiments, Compound I neat amorphous form is characterized as having a ¹³C SSNMR spectrum with a peak at 22.5±0.2 ppm. In some embodiments, Compound I neat amorphous form is characterized as having a ¹³C SSNMR spectrum with a peak at 19.5±0.2 ppm.

In some embodiments, Compound I neat amorphous form is characterized as having a ¹³C SSNMR spectrum with one, two, three, four, five, six, seven, eight, nine, ten, or more peaks selected from 163.8±0.2 ppm, 151.9±0.2 ppm, 137.6±0.2 ppm, 125.8±0.2 ppm, 120.8±0.2 ppm, 117.8±0.2 ppm, 77.3±0.2 ppm, 73.6±0.2 ppm, 34.5±0.2 ppm, 31.4±0.2 ppm, 26.3±0.2 ppm, 22.5±0.2 ppm, and 19.5±0.2 ppm. In some embodiments, Compound I neat amorphous form is characterized as having a ¹³C SSNMR spectrum with peaks at 163.8±0.2 ppm, 151.9±0.2 ppm, 137.6±0.2 ppm, 125.8±0.2 ppm, 120.8±0.2 ppm, 117.8±0.2 ppm, 77.3±0.2 ppm, 73.6±0.2 ppm, 34.5±0.2 ppm, 31.4±0.2 ppm, 26.3±0.2 ppm, 22.5±0.2 ppm, and 19.5±0.2 ppm.

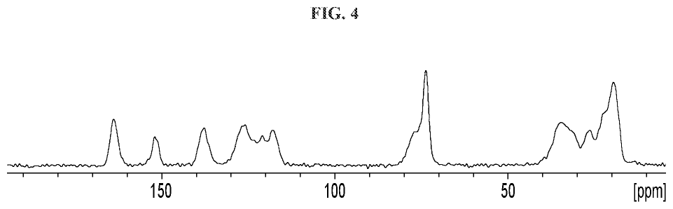

In some embodiments, Compound I neat amorphous form is characterized by a ¹³C SSNMR spectrum substantially similar to FIG. 4.

In some embodiments, Compound I neat amorphous form characterized as having a ¹⁹F SSNMR spectrum with a peak at −64.6±0.2 ppm. In some embodiments, Compound I neat amorphous form characterized as having a ¹⁹F SSNMR spectrum with a peak at −77.4±0.2 ppm.

In some embodiments, Compound I neat amorphous form C is characterized as having a ¹⁹F SSNMR spectrum with one or two peaks selected from −64.6±0.2 ppm and −77.4±0.2 ppm.

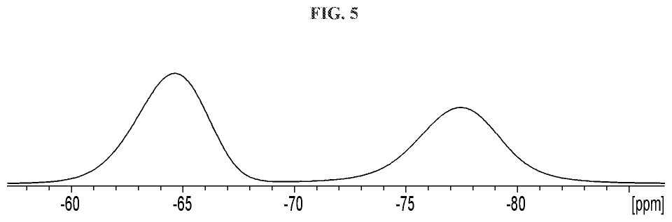

In some embodiments, Compound I neat amorphous form is characterized by a ¹⁹F SSNMR spectrum substantially similar to FIG. 5.

Another aspect of the invention provides a method of making Compound I neat amorphous form. In some embodiments, the method of making Compound I neat amorphous form comprises: (i) dissolving tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate in ethanol, (ii) adding 10% Pd/C, (iii) stirring at room temperature under hydrogen, (iv) isolating and evaporating the liquid phase, (v) redissolving in dichloromethane, (vi) cooling the solution in an ice bath and treating with trifluoroacetic acid, (viii) stirring at room temperature for 2 h, (ix) diluting the solution with heptane, evaporating, and drying to yield a solid, (x) dissolving the solid in dichloromethane and diluting with heptane, (xi) stirring the suspension at room temperature, (xii) filtering off the solids, (xiii) concentrating the mother liquor and purifying the resulting solid by reverse phase chromatography to yield Compound I neat amorphous form.

### B. Crystalline Compound I Neat Form A

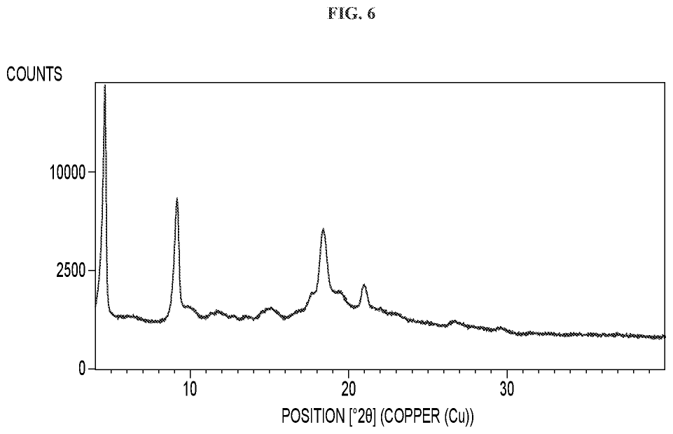

In some embodiments, the invention provides neat crystalline forms of Compound I. In some embodiments, the invention provides crystalline Compound I neat Form A. FIG. 6 provides an X-ray powder diffractogram of crystalline Compound I neat Form A.

In some embodiments, crystalline Compound I neat Form A is substantially pure. In some embodiments, crystalline Compound I neat Form A is substantially crystalline. In some embodiments, crystalline Compound I neat Form A is characterized by an X-ray powder diffractogram generated by an X-ray powder diffraction analysis with an incident beam of Cu Kα radiation.

In some embodiments, crystalline Compound I neat Form A is characterized by an X-ray powder diffractogram having a signal at 4.6±0.2 degrees two-theta. In some embodiments, crystalline Compound I neat Form A is characterized by an X-ray powder diffractogram having a signal at 20.8±0.2 degrees two-theta.

In some embodiments, crystalline Compound I neat Form A is characterized by an X-ray powder diffractogram having signals at one or two of 4.6±0.2 degrees two-theta and 20.8±0.2 degrees two-theta.

In some embodiments, crystalline Compound I neat Form A is characterized by an X-ray powder diffractogram having (a) signals at one or two of 4.6±0.2 degrees two-theta and 20.8±0.2 degrees two-theta, and (b) signals at one or two of 9.2±0.2 degrees two-theta, and 18.4±0.2 degrees two-theta.

In some embodiments, crystalline Compound I neat Form A is characterized by an X-ray powder diffractogram having signals at two, three, or four of 4.6±0.2 degrees two-theta, 9.2±0.2 degrees two-theta, 18.4±0.2 degrees two-theta, and 20.8±0.2 degrees two-theta.

In some embodiments, crystalline Compound I neat Form A is characterized by an X-ray powder diffractogram substantially similar to FIG. 6.

Another aspect of the invention provides a method of making crystalline Compound I neat Form A. In some embodiments, the method of making crystalline Compound I neat Form A comprises: (i) dissolving Compound I heptane solvate Form A in methanol, (ii) adding water, (iii) stirring at room temperature for five days, (iv) collecting the solids and drying under vacuum at 40° C. for 24 hours to yield crystalline Compound I neat Form A.

### C. Crystalline Compound I Neat Form B

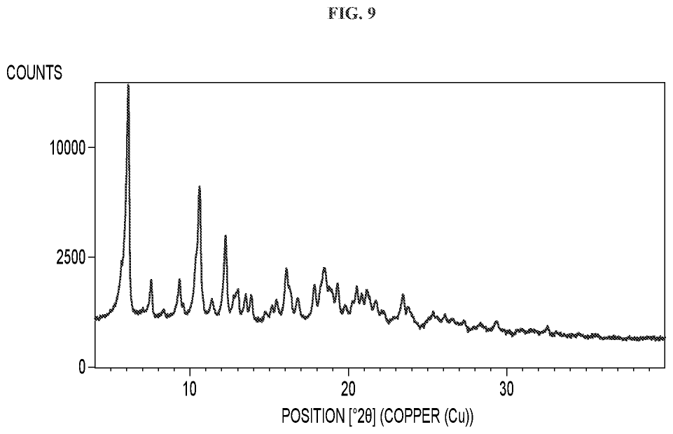

In some embodiments, the invention provides crystalline Compound I neat Form B. FIG. 9 provides an X-ray powder diffractogram of crystalline Compound I neat Form B.

In some embodiments, crystalline Compound I neat Form B is substantially pure. In some embodiments, crystalline Compound I neat Form B is substantially crystalline. In some embodiments, crystalline Compound I neat Form B is characterized by an X-ray powder diffractogram generated by an X-ray powder diffraction analysis with an incident beam of Cu Kα radiation.

In some embodiments, crystalline Compound I neat Form B is characterized by an X-ray powder diffractogram having a signal at 5.7±0.2 degrees two-theta. In some embodiments, crystalline Compound I neat Form B is characterized by an X-ray powder diffractogram having a signal at 6.1±0.2 degrees two-theta. In some embodiments, crystalline Compound I neat Form B is characterized by an X-ray powder diffractogram having a signal at 7.6±0.2 degrees two-theta. In some embodiments, crystalline Compound I neat Form B is characterized by an X-ray powder diffractogram having a signal at 10.3±0.2 degrees two-theta. In some embodiments, crystalline Compound I neat Form B is characterized by an X-ray powder diffractogram having a signal at 10.6±0.2 degrees two-theta. In some embodiments, crystalline Compound I neat Form B is characterized by an X-ray powder diffractogram having a signal at 12.3±0.2 degrees two-theta.

In some embodiments, crystalline Compound I neat Form B is characterized by an X-ray powder diffractogram having signals at one, two, three, four, five, or six of 5.7±0.2 degrees two-theta, 6.1±0.2 degrees two-theta, 7.6±0.2 degrees two-theta, 10.3±0.2 degrees two-theta, 10.6±0.2 degrees two-theta, and 12.3±0.2 degrees two-theta.

In some embodiments, crystalline Compound I neat Form B is characterized by an X-ray powder diffractogram having (a) signals at one, two, three, four, five, or six of 5.7±0.2 degrees two-theta, 6.1±0.2 degrees two-theta, 7.6±0.2 degrees two-theta, 10.3±0.2 degrees two-theta, 10.6±0.2 degrees two-theta, and 12.3±0.2 degrees two-theta, and (b) signals at one or two of 9.3±0.2 degrees two-theta, and 16.1±0.2 degrees two-theta.

In some embodiments, crystalline Compound I neat Form B is characterized by an X-ray powder diffractogram having signals at two, three, four, five, six, seven, or eight of 5.7±0.2 degrees two-theta, 6.1±0.2 degrees two-theta, 7.6±0.2 degrees two-theta, 9.3±0.2 degrees two-theta, 10.3±0.2 degrees two-theta, 10.6±0.2 degrees two-theta, 12.3±0.2 degrees two-theta, and 16.1±0.2 degrees two-theta.

In some embodiments, crystalline Compound I neat Form B is characterized by an X-ray powder diffractogram having signals at 5.7±0.2 degrees two-theta, 6.1±0.2 degrees two-theta, 7.6±0.2 degrees two-theta, 9.3±0.2 degrees two-theta, 10.3±0.2 degrees two-theta, 10.6±0.2 degrees two-theta, 12.3±0.2 degrees two-theta, and 16.1±0.2 degrees two-theta.

In some embodiments, crystalline Compound I neat Form B is characterized by an X-ray powder diffractogram substantially similar to FIG. 9.

In some embodiments, crystalline Compound I neat Form B is characterized as having a ¹³C SSNMR spectrum with a peak at 165.8±0.2 ppm. In some embodiments, crystalline Compound I neat Form B is characterized as having a ¹³C SSNMR spectrum with a peak at 154.2±0.2 ppm. In some embodiments, crystalline Compound I neat Form B is characterized as having a ¹³C SSNMR spectrum with a peak at 151.8±0.2 ppm. In some embodiments, crystalline Compound I neat Form B is characterized as having a ¹³C SSNMR spectrum with a peak at 140.1±0.2 ppm. In some embodiments, crystalline Compound I neat Form B is characterized as having a ¹³C SSNMR spectrum with a peak at 138.1±0.2 ppm. In some embodiments, crystalline Compound I neat Form B is characterized as having a ¹³C SSNMR spectrum with a peak at 136.2±0.2 ppm. In some embodiments, crystalline Compound I neat Form B is characterized as having a ¹³C SSNMR spectrum with a peak at 134.9±0.2 ppm. In some embodiments, crystalline Compound I neat Form B is characterized as having a ¹³C SSNMR spectrum with a peak at 131.7±0.2 ppm. In some embodiments, crystalline Compound I neat Form B is characterized as having a ¹³C SSNMR spectrum with a peak at 129.4±0.2 ppm. In some embodiments, crystalline Compound I neat Form B is characterized as having a ¹³C SSNMR spectrum with a peak at 125.5±0.2 ppm. In some embodiments, crystalline Compound I neat Form B is characterized as having a ¹³C SSNMR spectrum with a peak at 123.0±0.2 ppm. In some embodiments, crystalline Compound I neat Form B is characterized as having a ¹³C SSNMR spectrum with a peak at 120.2±0.2 ppm. In some embodiments, crystalline Compound I neat Form B is characterized as having a ¹³C SSNMR spectrum with a peak at 117.5±0.2 ppm. In some embodiments, crystalline Compound I neat Form B is characterized as having a ¹³C SSNMR spectrum with a peak at 78.3±0.2 ppm. In some embodiments, crystalline Compound I neat Form B is characterized as having a ¹³C SSNMR spectrum with a peak at 73.6±0.2 ppm. In some embodiments, crystalline Compound I neat Form B is characterized as having a ¹³C SSNMR spectrum with a peak at 37.6±0.2 ppm. In some embodiments, crystalline Compound I neat Form B is characterized as having a ¹³C SSNMR spectrum with a peak at 34.0±0.2 ppm. In some embodiments, crystalline Compound I neat Form B is characterized as having a ¹³C SSNMR spectrum with a peak at 29.9±0.2 ppm. In some embodiments, crystalline Compound I neat Form B is characterized as having a ¹³C SSNMR spectrum with a peak at 27.3±0.2 ppm. In some embodiments, crystalline Compound I neat Form B is characterized as having a ¹³C SSNMR spectrum with a peak at 22.7±0.2 ppm. In some embodiments, crystalline Compound I neat Form B is characterized as having a ¹³C SSNMR spectrum with a peak at 21.1±0.2 ppm. In some embodiments, crystalline Compound I neat Form B is characterized as having a ¹³C SSNMR spectrum with a peak at 18.9±0.2 ppm.

In some embodiments, crystalline Compound I neat Form B is characterized as having a ¹³C SSNMR spectrum with one, two, three, four, five, six, seven, eight, nine, ten, or more peaks selected from 165.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm. In some embodiments, crystalline Compound I neat Form B is characterized as having a ¹³C SSNMR spectrum with peaks at 165.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm.

In some embodiments, crystalline Compound I neat Form B is characterized as having a ¹³C SSNMR spectrum with (a) one, two, three, four, five, six, seven, eight, nine, ten, or more peaks selected from 165.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm, and (b) one, two, or three peaks selected from 164.7±0.2 ppm, 163.8±0.2 ppm, and 74.4±0.2 ppm.

In some embodiments, crystalline Compound I neat Form B is characterized as having a ¹³C SSNMR spectrum with four, five, six, seven, eight, nine, ten, or more peaks selected from 165.8±0.2 ppm, 164.7±0.2 ppm, 163.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 74.4±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm.

In some embodiments, crystalline Compound I neat Form B is characterized as having a ¹³C SSNMR spectrum with peaks at 165.8±0.2 ppm, 164.7±0.2 ppm, 163.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 74.4±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm.

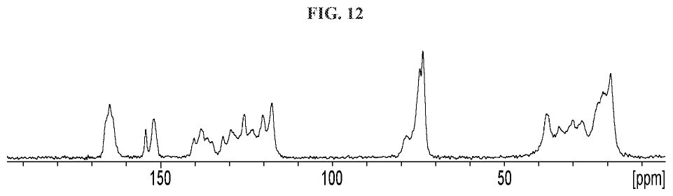

In some embodiments, crystalline Compound I neat Form B is characterized by a ¹³C SSNMR spectrum substantially similar to FIG. 12.

In some embodiments, crystalline Compound I neat Form B is characterized as having a ¹⁹F SSNMR spectrum with a peak at −64.3±0.2 ppm. In some embodiments, crystalline Compound I neat Form B is characterized as having a ¹⁹F SSNMR spectrum with a peak at −65.9±0.2 ppm. In some embodiments, crystalline Compound I neat Form B is characterized as having a ¹⁹F SSNMR spectrum with a peak at −76.5±0.2 ppm.

In some embodiments, crystalline Compound I neat Form B is characterized as having a ¹⁹F SSNMR spectrum with one or two peaks selected from −64.3±0.2 ppm, −65.9±0.2 ppm, and −76.5±0.2 ppm. In some embodiments, crystalline Compound I neat Form B is characterized as having a ¹⁹F SSNMR spectrum with peaks at −64.3±0.2 ppm, −65.9±0.2 ppm, and −76.5±0.2 ppm.

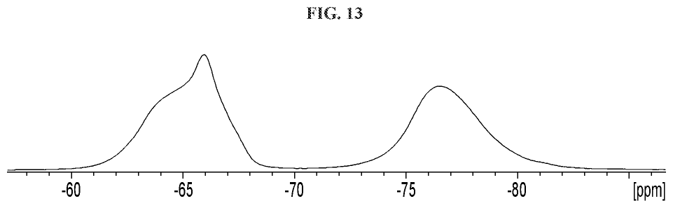

In some embodiments, crystalline Compound I neat Form B is characterized by a ¹⁹F SSNMR spectrum substantially similar to FIG. 13.

Another aspect of the invention provides a method of making crystalline Compound I neat Form B. In some embodiments, the method of making crystalline Compound I neat Form B comprises: (i) dissolving Compound I heptane solvate Form A in dichloromethane at room temperature, and (ii) evaporating the dichloromethanat slowly at room temperature to yield crystalline Compound I neat Form B.

### D. Crystalline Compound I Hemihydrate Form C

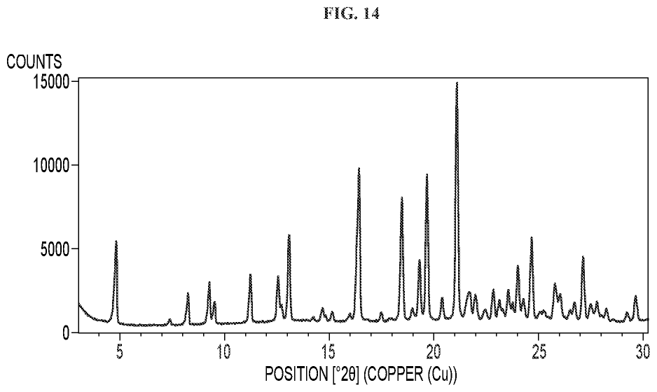

In some embodiments, the invention provides crystalline Compound I hemihydrate Form C. FIG. 14 provides an X-ray powder diffractogram of crystalline Compound I hemihydrate Form C.

In some embodiments, crystalline Compound I hemihydrate Form C is substantially pure. In some embodiments, crystalline Compound I hemihydrate Form C is substantially crystalline. In some embodiments, crystalline Compound I hemihydrate Form C is characterized by an X-ray powder diffractogram generated by an X-ray powder diffraction analysis with an incident beam of Cu Kα radiation.

In some embodiments, crystalline Compound I hemihydrate Form C is characterized by an X-ray powder diffractogram having a signal at 4.8±0.2 degrees two-theta. In some embodiments, crystalline Compound I hemihydrate Form C is characterized by an X-ray powder diffractogram having a signal at 8.2±0.2 degrees two-theta. In some embodiments, crystalline Compound I hemihydrate Form C is characterized by an X-ray powder diffractogram having a signal at 9.3±0.2 degrees two-theta. In some embodiments, crystalline Compound I hemihydrate Form C is characterized by an X-ray powder diffractogram having a signal at 11.2±0.2 degrees two-theta. In some embodiments, crystalline Compound I hemihydrate Form C is characterized by an X-ray powder diffractogram having a signal at 12.5±0.2 degrees two-theta. In some embodiments, crystalline Compound I hemihydrate Form C is characterized by an X-ray powder diffractogram having a signal at 13.1±0.2 degrees two-theta. In some embodiments, crystalline Compound I hemihydrate Form C is characterized by an X-ray powder diffractogram having a signal at 16.4±0.2 degrees two-theta. In some embodiments, crystalline Compound I hemihydrate Form C is characterized by an X-ray powder diffractogram having a signal at 18.4±0.2 degrees two-theta. In some embodiments, crystalline Compound I hemihydrate Form C is characterized by an X-ray powder diffractogram having a signal at 19.3±0.2 degrees two-theta. In some embodiments, crystalline Compound I hemihydrate Form C is characterized by an X-ray powder diffractogram having a signal at 19.6±0.2 degrees two-theta. In some embodiments, crystalline Compound I hemihydrate Form C is characterized by an X-ray powder diffractogram having a signal at 21.1±0.2 degrees two-theta. In some embodiments, crystalline Compound I hemihydrate Form C is characterized by an X-ray powder diffractogram having a signal at 21.6±0.2 degrees two-theta. In some embodiments, crystalline Compound I hemihydrate Form C is characterized by an X-ray powder diffractogram having a signal at 22.8±0.2 degrees two-theta. In some embodiments, crystalline Compound I hemihydrate Form C is characterized by an X-ray powder diffractogram having a signal at 23.5±0.2 degrees two-theta. In some embodiments, crystalline Compound I hemihydrate Form C is characterized by an X-ray powder diffractogram having a signal at 24±0.2 degrees two-theta. In some embodiments, crystalline Compound I hemihydrate Form C is characterized by an X-ray powder diffractogram having a signal at 24.6±0.2 degrees two-theta. In some embodiments, crystalline Compound I hemihydrate Form C is characterized by an X-ray powder diffractogram having a signal at 25.8±0.2 degrees two-theta. In some embodiments, crystalline Compound I hemihydrate Form C is characterized by an X-ray powder diffractogram having a signal at 27.1±0.2 degrees two-theta. In some embodiments, crystalline Compound I hemihydrate Form C is characterized by an X-ray powder diffractogram having a signal at 29.6±0.2 degrees two-theta. In some embodiments, crystalline Compound I hemihydrate Form C is characterized by an X-ray powder diffractogram having a signal at 33.4±0.2 degrees two-theta.

In some embodiments, crystalline Compound I hemihydrate Form C is characterized by an X-ray powder diffractogram having signals at one, two, three, or four of 4.8±0.2 degrees two-theta, 16.4±0.2 degrees two-theta, 19.3±0.2 degrees two-theta, and 21.1±0.2 degrees two-theta.

In some embodiments, crystalline Compound I hemihydrate Form C is characterized by an X-ray powder diffractogram having signals at one, two, three, four, or five of 13.1±0.2 degrees two-theta, 16.4±0.2 degrees two-theta, 18.4±0.2 degrees two-theta, 19.6±0.2 degrees two-theta, and 21.1±0.2 degrees two-theta.

In some embodiments, crystalline Compound I hemihydrate Form C is characterized by an X-ray powder diffractogram having signals at one, two, three, four, five, six, seven, eight, nine, or ten of 4.8±0.2 degrees two-theta, 13.1±0.2 degrees two-theta, 16.4±0.2 degrees two-theta, 18.4±0.2 degrees two-theta, 19.3±0.2 degrees two-theta, 19.6±0.2 degrees two-theta, 21.1±0.2 degrees two-theta, 24.0±0.2 degrees two-theta, 24.6±0.2 degrees two-theta, and 27.1±0.2 degrees two-theta.

In some embodiments, crystalline Compound I hemihydrate Form C is characterized by an X-ray powder diffractogram having signals at one, two, three, four, five, six, seven, eight, nine, ten, or more of 4.8±0.2 degrees two-theta, 8.2±0.2 degrees two-theta, 9.3±0.2 degrees two-theta, 11.2±0.2 degrees two-theta, 12.5±0.2 degrees two-theta, 13.1±0.2 degrees two-theta, 16.4±0.2 degrees two-theta, 18.4±0.2 degrees two-theta, 19.3±0.2 degrees two-theta, 19.6±0.2 degrees two-theta, 21.1±0.2 degrees two-theta, 21.6±0.2 degrees two-theta, 22.8±0.2 degrees two-theta, 23.5±0.2 degrees two-theta, 24.0±0.2 degrees two-theta, 24.6±0.2 degrees two-theta, 25.8±0.2 degrees two-theta, 27.1±0.2 degrees two-theta, 29.6±0.2 degrees two-theta, and 33.4±0.2 degrees two-theta. In some embodiments, crystalline Compound I hemihydrate Form C is characterized by an X-ray powder diffractogram having signals at 4.8±0.2 degrees two-theta, 8.2±0.2 degrees two-theta, 9.3±0.2 degrees two-theta, 11.2±0.2 degrees two-theta, 12.5±0.2 degrees two-theta, 13.1±0.2 degrees two-theta, 16.4±0.2 degrees two-theta, 18.4±0.2 degrees two-theta, 19.3±0.2 degrees two-theta, 19.6±0.2 degrees two-theta, 21.1±0.2 degrees two-theta, 21.6±0.2 degrees two-theta, 22.8±0.2 degrees two-theta, 23.5±0.2 degrees two-theta, 24±0.2 degrees two-theta, 24.6±0.2 degrees two-theta, 25.8±0.2 degrees two-theta, 27.1±0.2 degrees two-theta, 29.6±0.2 degrees two-theta, and 33.4±0.2 degrees two-theta.

In some embodiments, crystalline Compound I hemihydrate Form C is characterized by an X-ray powder diffractogram substantially similar to FIG. 14.

In some embodiments, crystalline Compound I hemihydrate Form C is characterized as having a ¹³C SSNMR spectrum with a peak at 163.8±0.2 ppm. In some embodiments, crystalline Compound I hemihydrate Form C is characterized as having a ¹³C SSNMR spectrum with a peak at 151.3±0.2 ppm. In some embodiments, crystalline Compound I hemihydrate Form C is characterized as having a ¹³C SSNMR spectrum with a peak at 139.1±0.2 ppm. In some embodiments, crystalline Compound I hemihydrate Form C is characterized as having a ¹³C SSNMR spectrum with a peak at 137.7±0.2 ppm. In some embodiments, crystalline Compound I hemihydrate Form C is characterized as having a ¹³C SSNMR spectrum with a peak at 127.2±0.2 ppm. In some embodiments, crystalline Compound I hemihydrate Form C is characterized as having a ¹³C SSNMR spectrum with a peak at 125.8±0.2 ppm. In some embodiments, crystalline Compound I hemihydrate Form C is characterized as having a ¹³C SSNMR spectrum with a peak at 119.9±0.2 ppm. In some embodiments, crystalline Compound I hemihydrate Form C is characterized as having a ¹³C SSNMR spectrum with a peak at 118.4±0.2 ppm. In some embodiments, crystalline Compound I hemihydrate Form C is characterized as having a ¹³C SSNMR spectrum with a peak at 75.6±0.2 ppm. In some embodiments, crystalline Compound I hemihydrate Form C is characterized as having a ¹³C SSNMR spectrum with a peak at 73.6±0.2 ppm. In some embodiments, crystalline Compound I hemihydrate Form C is characterized as having a ¹³C SSNMR spectrum with a peak at 35.8±0.2 ppm. In some embodiments, crystalline Compound I hemihydrate Form C is characterized as having a ¹³C SSNMR spectrum with a peak at 32.2±0.2 ppm. In some embodiments, crystalline Compound I hemihydrate Form C is characterized as having a ¹³C SSNMR spectrum with a peak at 29.6±0.2 ppm. In some embodiments, crystalline Compound I hemihydrate Form C is characterized as having a ¹³C SSNMR spectrum with a peak at 24.6±0.2 ppm. In some embodiments, crystalline Compound I hemihydrate Form C is characterized as having a ¹³C SSNMR spectrum with a peak at 22.1±0.2 ppm. In some embodiments, crystalline Compound I hemihydrate Form C is characterized as having a ¹³C SSNMR spectrum with a peak at 19.2±0.2 ppm.

In some embodiments, crystalline Compound I hemihydrate Form C is characterized as having a ¹³C SSNMR spectrum with one, two, three, four, five, six, seven, eight, nine, ten, or more peaks selected from 163.8±0.2 ppm, 151.3±0.2 ppm, 139.1±0.2 ppm, 137.7±0.2 ppm, 127.2±0.2 ppm, 125.8±0.2 ppm, 119.9±0.2 ppm, 118.4±0.2 ppm, 75.6±0.2 ppm, 73.6±0.2 ppm, 35.8±0.2 ppm, 32.2±0.2 ppm, 29.6±0.2 ppm, 24.6±0.2 ppm, 22.1±0.2 ppm, and 19.2±0.2 ppm. In some embodiments, crystalline Compound I hemihydrate Form C is characterized as having a ¹³C SSNMR spectrum with peaks at 163.8±0.2 ppm, 151.3±0.2 ppm, 139.1±0.2 ppm, 137.7±0.2 ppm, 127.2±0.2 ppm, 125.8±0.2 ppm, 119.9±0.2 ppm, 118.4±0.2 ppm, 75.6±0.2 ppm, 73.6±0.2 ppm, 35.8±0.2 ppm, 32.2±0.2 ppm, 29.6±0.2 ppm, 24.6±0.2 ppm, 22.1±0.2 ppm, and 19.2±0.2 ppm.

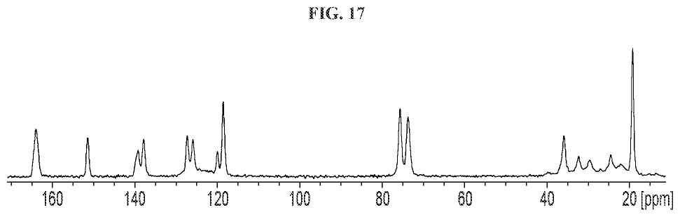

In some embodiments, crystalline Compound I hemihydrate Form C is characterized by a ¹³C SSNMR spectrum substantially similar to FIG. 17.

In some embodiments, crystalline Compound I hemihydrate Form C is characterized as having a ¹⁹F SSNMR spectrum with a peak at −65.5±0.2 ppm. In some embodiments, crystalline Compound I hemihydrate Form C is characterized as having a ¹⁹F SSNMR spectrum with a peak at −77.4±0.2 ppm. In some embodiments, crystalline Compound I hemihydrate Form C is characterized as having a ¹⁹F SSNMR spectrum with peaks at −65.5±0.2 ppm and −77.4±0.2 ppm.

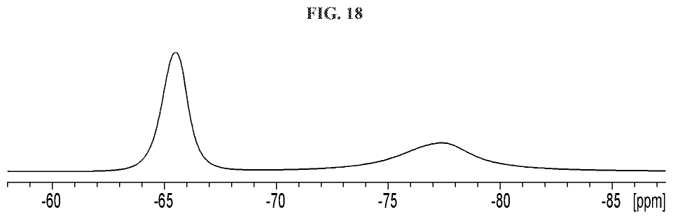

In some embodiments, crystalline Compound I hemihydrate Form C is characterized by a ¹⁹F SSNMR spectrum substantially similar to FIG. 18.

In some embodiments, crystalline Compound I hemihydrate Form C is characterized by a monoclinic crystal system, P 2₁ space group, and the following unit cell dimensions measured at 100 K on a Bruker diffractometer utilizing Cu K_{α} radiation (λ=1.54178 Å):

| | | | |
|---|---|---|---|
| a | 12.1 ± 0.1 Å | α | 90° |
| b | 8.6 ± 0.1 Å | β | 98.2 ± 0.1° |
| c | 18.9 ± 0.1 Å | γ | 90°. |

Another aspect of the invention provides a method of making crystalline Compound I hemihydrate Form C. In some embodiments, the method of making crystalline Compound I hemihydrate Form C comprises: (i) dissolving Compound I in ethanol at 25° C., (ii) adding water over 10-12 hours (ethanol to water ratio approximately 1:4 v/v), (iii) heating the slurry to 60° C. for 4 hours, (iv) cooling the slurry to 20° C. over 3 hours, (v) stirring for at least 2 hours, (vi) filtering the solids and washing with an ethanol/water solution (1:4 v/v), (vii) drying the solids in a vacuum oven at 50° C. with a slight nitrogen bleed to yield crystalline Compound I hemihydrate Form C.

### E. Crystalline Compound I Neat Form D

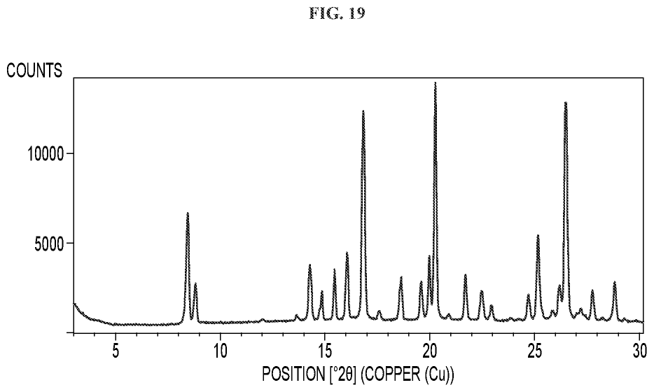

In some embodiments, the invention provides crystalline Compound I neat Form D. FIG. 19 provides an X-ray powder diffractogram of crystalline Compound I neat Form D.

In some embodiments, crystalline Compound I neat Form D is substantially pure. In some embodiments, crystalline Compound I neat Form D is substantially crystalline. In some embodiments, crystalline Compound I neat Form D is characterized by an X-ray powder diffractogram generated by an X-ray powder diffraction analysis with an incident beam of Cu Kα radiation.

In some embodiments, crystalline Compound I neat Form D is characterized by an X-ray powder diffractogram having a signal at 8.4±0.2 degrees two-theta. In some embodiments, crystalline Compound I neat Form D is characterized by an X-ray powder diffractogram having a signal at 8.8±0.2 degrees two-theta. In some embodiments, crystalline Compound I neat Form D is characterized by an X-ray powder diffractogram having a signal at 14.3±0.2 degrees two-theta. In some embodiments, crystalline Compound I neat Form D is characterized by an X-ray powder diffractogram having a signal at 14.8±0.2 degrees two-theta. In some embodiments, crystalline Compound I neat Form D is characterized by an X-ray powder diffractogram having a signal at 15.4±0.2 degrees two-theta. In some embodiments, crystalline Compound I neat Form D is characterized by an X-ray powder diffractogram having a signal at 16.77±0.2 degrees two-theta. In some embodiments, crystalline Compound I neat Form D is characterized by an X-ray powder diffractogram having a signal at 16.85±0.2 degrees two-theta. In some embodiments, crystalline Compound I neat Form D is characterized by an X-ray powder diffractogram having a signal at 19.6±0.2 degrees two-theta. In some embodiments, crystalline Compound I neat Form D is characterized by an X-ray powder diffractogram having a signal at 20.0±0.2 degrees two-theta. In some embodiments, crystalline Compound I neat Form D is characterized by an X-ray powder diffractogram having a signal at 20.3±0.2 degrees two-theta. In some embodiments, crystalline Compound I neat Form D is characterized by an X-ray powder diffractogram having a signal at 21.7±0.2 degrees two-theta. In some embodiments, crystalline Compound I neat Form D is characterized by an X-ray powder diffractogram having a signal at 22.5±0.2 degrees two-theta. In some embodiments, crystalline Compound I neat Form D is characterized by an X-ray powder diffractogram having a signal at 24.7±0.2 degrees two-theta. In some embodiments, crystalline Compound I neat Form D is characterized by an X-ray powder diffractogram having a signal at 25.2±0.2 degrees two-theta. In some embodiments, crystalline Compound I neat Form D is characterized by an X-ray powder diffractogram having a signal at 26.2±0.2 degrees two-theta. In some embodiments, crystalline Compound I neat Form D is characterized by an X-ray powder diffractogram having a signal at 26.45±0.2 degrees two-theta. In some embodiments, crystalline Compound I neat Form D is characterized by an X-ray powder diffractogram having a signal at 26.52±0.2 degrees two-theta. In some embodiments, crystalline Compound I neat Form D is characterized by an X-ray powder diffractogram having a signal at 27.8±0.2 degrees two-theta. In some embodiments, crystalline Compound I neat Form D is characterized by an X-ray powder diffractogram having a signal at 28.8±0.2 degrees two-theta.

In some embodiments, crystalline Compound I neat Form D is characterized by an X-ray powder diffractogram having signals at one, two, three, four, five, six, seven, eight, nine, ten, or more of 8.4±0.2 degrees two-theta, 8.8±0.2 degrees two-theta, 14.3±0.2 degrees two-theta, 14.8±0.2 degrees two-theta, 15.4±0.2 degrees two-theta, 16.77±0.2 degrees two-theta, 16.85±0.2 degrees two-theta, 19.6±0.2 degrees two-theta, 20.0±0.2 degrees two-theta, 20.3±0.2 degrees two-theta, 21.7±0.2 degrees two-theta, 22.5±0.2 degrees two-theta, 24.7±0.2 degrees two-theta, 25.2±0.2 degrees two-theta, 26.2±0.2 degrees two-theta, 26.45±0.2 degrees two-theta, 26.52±0.2 degrees two-theta, 27.8±0.2 degrees two-theta, and 28.8±0.2 degrees two-theta.

In some embodiments, crystalline Compound I neat Form D is characterized by an X-ray powder diffractogram having (a) signals at one, two, three, four, five, six, seven, eight, nine, ten, or more of 8.4±0.2 degrees two-theta, 8.8±0.2 degrees two-theta, 14.3±0.2 degrees two-theta, 14.8±0.2 degrees two-theta, 15.4±0.2 degrees two-theta, 16.77±0.2 degrees two-theta, 16.85±0.2 degrees two-theta, 19.6±0.2 degrees two-theta, 20.0±0.2 degrees two-theta, 20.3±0.2 degrees two-theta, 21.7±0.2 degrees two-theta, 22.5±0.2 degrees two-theta, 24.7±0.2 degrees two-theta, 25.2±0.2 degrees two-theta, 26.2±0.2 degrees two-theta, 26.45±0.2 degrees two-theta, 26.52±0.2 degrees two-theta, 27.8±0.2 degrees two-theta, and 28.8±0.2 degrees two-theta, and (b) signals at one, two, or three of 16.0±0.2 degrees two-theta, 18.55±0.2 degrees two-theta, and 18.64±0.2 degrees two-theta.

In some embodiments, crystalline Compound I neat Form D is characterized by an X-ray powder diffractogram having signals at three, four, five, six, seven, eight, nine, ten, or more of 8.4±0.2 degrees two-theta, 8.8±0.2 degrees two-theta, 14.3±0.2 degrees two-theta, 14.8±0.2 degrees two-theta, 15.4±0.2 degrees two-theta, 16.0±0.2 degrees two-theta, 16.77±0.2 degrees two-theta, 16.85±0.2 degrees two-theta, 18.55±0.2 degrees two-theta, 18.64±0.2 degrees two-theta, 19.6±0.2 degrees two-theta, 20.0±0.2 degrees two-theta, 20.3±0.2 degrees two-theta, 21.7±0.2 degrees two-theta, 22.5±0.2 degrees two-theta, 24.7±0.2 degrees two-theta, 25.2±0.2 degrees two-theta, 26.2±0.2 degrees two-theta, 26.45±0.2 degrees two-theta, 26.52±0.2 degrees two-theta, 27.8±0.2 degrees two-theta, and 28.8±0.2 degrees two-theta.

In some embodiments, crystalline Compound I neat Form D is characterized by an X-ray powder diffractogram having signals at 8.4±0.2 degrees two-theta, 8.8±0.2 degrees two-theta, 14.3±0.2 degrees two-theta, 14.8±0.2 degrees two-theta, 15.4±0.2 degrees two-theta, 16.0±0.2 degrees two-theta, 16.77±0.2 degrees two-theta, 16.85±0.2 degrees two-theta, 18.55±0.2 degrees two-theta, 18.64±0.2 degrees two-theta, 19.6±0.2 degrees two-theta, 20.0±0.2 degrees two-theta, 20.3±0.2 degrees two-theta, 21.7±0.2 degrees two-theta, 22.5±0.2 degrees two-theta, 24.7±0.2 degrees two-theta, 25.2±0.2 degrees two-theta, 26.2±0.2 degrees two-theta, 26.45±0.2 degrees two-theta, 26.52±0.2 degrees two-theta, 27.8±0.2 degrees two-theta, and 28.8±0.2 degrees two-theta.

In some embodiments, crystalline Compound I neat Form D is characterized by an X-ray powder diffractogram substantially similar to FIG. 19.

In some embodiments, crystalline Compound I neat Form D is characterized as having a ¹³C SSNMR spectrum with a peak at 152.2±0.2 ppm. In some embodiments, crystalline Compound I neat Form D is characterized as having a ¹³C SSNMR spectrum with a peak at 137.7±0.2 ppm. In some embodiments, crystalline Compound I neat Form D is characterized as having a ¹³C SSNMR spectrum with a peak at 127.3±0.2 ppm. In some embodiments, crystalline Compound I neat Form D is characterized as having a ¹³C SSNMR spectrum with a peak at 120.8±0.2 ppm. In some embodiments, crystalline Compound I neat Form D is characterized as having a ¹³C SSNMR spectrum with a peak at 118.1±0.2 ppm. In some embodiments, crystalline Compound I neat Form D is characterized as having a ¹³C SSNMR spectrum with a peak at 75.7±0.2 ppm. In some embodiments, crystalline Compound I neat Form D is characterized as having a ¹³C SSNMR spectrum with a peak at 35.9±0.2 ppm. In some embodiments, crystalline Compound I neat Form D is characterized as having a ¹³C SSNMR spectrum with a peak at 30.4±0.2 ppm. In some embodiments, crystalline Compound I neat Form D is characterized as having a ¹³C SSNMR spectrum with a peak at 22.1±0.2 ppm. In some embodiments, crystalline Compound I neat Form D is characterized as having a ¹³C SSNMR spectrum with a peak at 17.7±0.2 ppm.

In some embodiments, crystalline Compound I neat Form D is characterized as having a ¹³C SSNMR spectrum with one, two, three, four, five, six, seven, eight, or nine peaks selected from 152.2±0.2 ppm, 137.7±0.2 ppm, 127.3±0.2 ppm, 120.8±0.2 ppm, 118.1±0.2 ppm, 75.7±0.2 ppm, 35.9±0.2 ppm, 30.4±0.2 ppm, 22.1±0.2 ppm, and 17.7±0.2 ppm.

In some embodiments, crystalline Compound I neat Form D is characterized as having a ¹³C SSNMR spectrum with (a) one, two, three, four, five, six, seven, eight, nine, or ten peaks selected from 152.2±0.2 ppm, 137.7±0.2 ppm, 127.3±0.2 ppm, 120.8±0.2 ppm, 118.1±0.2 ppm, 75.7±0.2 ppm, 35.9±0.2 ppm, 30.4±0.2 ppm, 22.1±0.2 ppm, and 17.7±0.2 ppm, and (b) one, two, or three peaks selected from 164.6±0.2 ppm, 163.8±0.2 ppm, and 74.2±0.2 ppm.

In some embodiments, crystalline Compound I neat Form D is characterized as having a ¹³C SSNMR spectrum with four, five, six, seven, eight, nine, ten, or more peaks selected from 164.6±0.2 ppm, 163.8±0.2 ppm, 152.2±0.2 ppm, 137.7±0.2 ppm, 127.3±0.2 ppm, 120.8±0.2 ppm, 118.1±0.2 ppm, 75.7±0.2 ppm, 74.2±0.2 ppm, 35.9±0.2 ppm, 30.4±0.2 ppm, 22.1±0.2 ppm, and 17.7±0.2 ppm.

In some embodiments, crystalline Compound I neat Form D is characterized as having a ¹³C SSNMR spectrum with peaks at 164.6±0.2 ppm, 163.8±0.2 ppm, 152.2±0.2 ppm, 137.7±0.2 ppm, 127.3±0.2 ppm, 120.8±0.2 ppm, 118.1±0.2 ppm, 75.7±0.2 ppm, 74.2±0.2 ppm, 35.9±0.2 ppm, 30.4±0.2 ppm, 22.1±0.2 ppm, and 17.7±0.2 ppm.

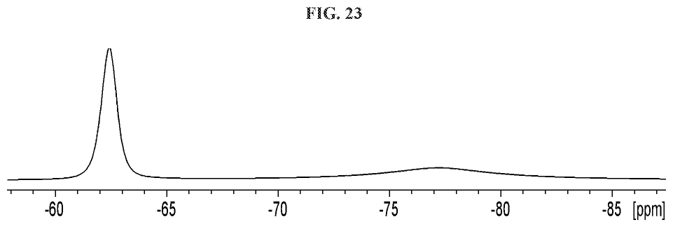

In some embodiments, crystalline Compound I neat Form D is characterized by a ¹³C SSNMR spectrum substantially similar to FIG. 23.

In some embodiments, crystalline Compound I neat Form D is characterized as having a ¹⁹F SSNMR spectrum with a peak at −62.4±0.2 ppm. In some embodiments, crystalline Compound I neat Form D is characterized as having a ¹⁹F SSNMR spectrum with a peak at −77.2±0.2 ppm. In some embodiments, crystalline Compound I neat Form D is characterized as having a ¹⁹F SSNMR spectrum with a peak at −62.4±0.2 ppm and −77.2±0.2 ppm.

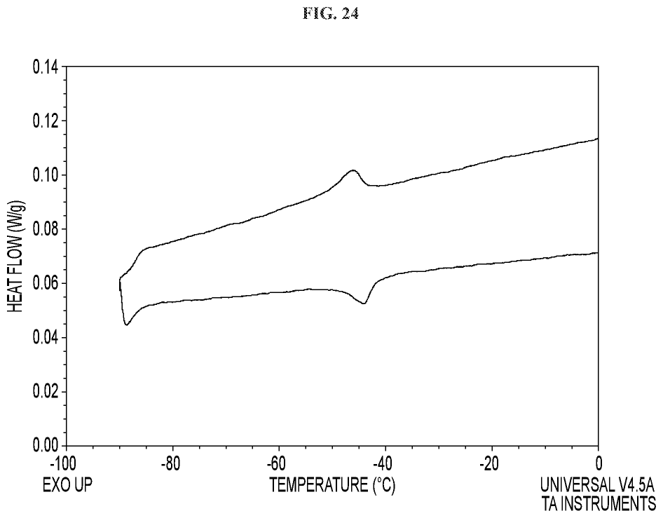

In some embodiments, crystalline Compound I neat Form D is characterized by a ¹⁹F SSNMR spectrum substantially similar to FIG. 24.

In some embodiments, crystalline Compound I neat Form D is characterized by a monoclinic crystal system, P 2₁ space group, and the following unit cell dimensions measured at 250 K on a Bruker diffractometer utilizing Cu K_{α} radiation (λ=1.54178 Å):

| | | | |
|---|---|---|---|
| a | 7.9 ± 0.1 Å | α | 90° |
| b | 11.5 ± 0.1 Å | β | 90.02 ± 0.10° |
| c | 21.0 ± 0.2 Å | γ | 90°. |

Another aspect of the invention provides a method of making crystalline Compound I neat Form D. In some embodiments, the method of making crystalline Compound I neat Form D comprises: (i) dissolving crystalline Compound I hemihydrate Form C in ethanol, (ii) placing the solution under nitrogen for a half hour, and (iii) placing the solution in an oven at 80° C. for ˜5 days to yield crystalline Compound I neat Form D. In some embodiments, the method of making crystalline Compound I neat Form D comprises: (i) slurrying Compound I hemihydrate Form C in n-heptane, (ii) heating the slurry to 85° C., (iii) adding a seed of crystalline Compound I neat Form D, (iv) holding the slurry at 85+5° C., (v) cooling the slurry to 65° C. over 4 hours, (vi) collecting the solids and washing the solids with n-heptane, and (vii) drying the solids in a vacuum oven at 50° C. with a slight nitrogen bleed to yield crystalline Compound I neat Form D.

### F. Crystalline Compound I Neat Form E

In some embodiments, the invention provides crystalline Compound I neat Form E.

In some embodiments, crystalline Compound I neat Form E is characterized by a orthorhombic crystal system, P 2₁2₁2₁ space group, and the following unit cell dimensions measured at 100 K on a Bruker diffractometer utilizing Cu K_{α} radiation (λ=1.54178 Å):

| | | | |
|---|---|---|---|
| a | 8.3 ± 0.1 Å | α | 90° |
| b | 11.2 ± 0.1 Å | β | 90° |
| c | 20.2 ± 0.1 Å | γ | 90°. |

Another aspect of the invention provides a method of making crystalline Compound I neat Form E. In some embodiments, the method of making crystalline Compound I neat Form E comprises cooling crystalline Compound I neat Form D to a temperature below −40° C. to yield crystalline Compound I neat Form E.

### G. Crystalline Compound I Acetic Acid Solvate

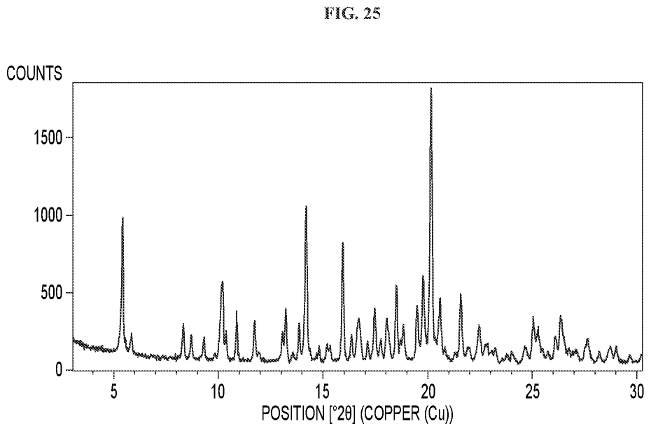

In some embodiments, the invention provides crystalline Compound I acetic acid solvate. FIG. 25 provides an X-ray powder diffractogram of crystalline Compound I acetic acid solvate.

In some embodiments, crystalline Compound I acetic acid solvate is substantially pure. In some embodiments, crystalline Compound I acetic acid solvate is substantially crystalline. In some embodiments, crystalline Compound I acetic acid solvate is characterized by an X-ray powder diffractogram generated by an X-ray powder diffraction analysis with an incident beam of Cu Kα radiation.

In some embodiments, crystalline Compound I acetic acid solvate is characterized by an X-ray powder diffractogram having a signal at 5.4±0.2 degrees two-theta. In some embodiments, crystalline Compound I acetic acid solvate is characterized by an X-ray powder diffractogram having a signal at 8.3±0.2 degrees two-theta. In some embodiments, crystalline Compound I acetic acid solvate is characterized by an X-ray powder diffractogram having a signal at 10.2±0.2 degrees two-theta. In some embodiments, crystalline Compound I acetic acid solvate is characterized by an X-ray powder diffractogram having a signal at 10.4±0.2 degrees two-theta. In some embodiments, crystalline Compound I acetic acid solvate is characterized by an X-ray powder diffractogram having a signal at 10.9±0.2 degrees two-theta. In some embodiments, crystalline Compound I acetic acid solvate is characterized by an X-ray powder diffractogram having a signal at 11.7±0.2 degrees two-theta. In some embodiments, crystalline Compound I acetic acid solvate is characterized by an X-ray powder diffractogram having a signal at 13.1±0.2 degrees two-theta. In some embodiments, crystalline Compound I acetic acid solvate is characterized by an X-ray powder diffractogram having a signal at 13.2±0.2 degrees two-theta. In some embodiments, crystalline Compound I acetic acid solvate is characterized by an X-ray powder diffractogram having a signal at 13.8±0.2 degrees two-theta. In some embodiments, crystalline Compound I acetic acid solvate is characterized by an X-ray powder diffractogram having a signal at 14.2±0.2 degrees two-theta. In some embodiments, crystalline Compound I acetic acid solvate is characterized by an X-ray powder diffractogram having a signal at 15.9±0.2 degrees two-theta. In some embodiments, crystalline Compound I acetic acid solvate is characterized by an X-ray powder diffractogram having a signal at 16.7±0.2 degrees two-theta. In some embodiments, crystalline Compound I acetic acid solvate is characterized by an X-ray powder diffractogram having a signal at 17.5±0.2 degrees two-theta. In some embodiments, crystalline Compound I acetic acid solvate is characterized by an X-ray powder diffractogram having a signal at 18.0±0.2 degrees two-theta. In some embodiments, crystalline Compound I acetic acid solvate is characterized by an X-ray powder diffractogram having a signal at 18.5±0.2 degrees two-theta. In some embodiments, crystalline Compound I acetic acid solvate is characterized by an X-ray powder diffractogram having a signal at 18.9±0.2 degrees two-theta. In some embodiments, crystalline Compound I acetic acid solvate is characterized by an X-ray powder diffractogram having a signal at 19.5±0.2 degrees two-theta. In some embodiments, crystalline Compound I acetic acid solvate is characterized by an X-ray powder diffractogram having a signal at 19.8±0.2 degrees two-theta. In some embodiments, crystalline Compound I acetic acid solvate is characterized by an X-ray powder diffractogram having a signal at 20.2±0.2 degrees two-theta. In some embodiments, crystalline Compound I acetic acid solvate is characterized by an X-ray powder diffractogram having a signal at 20.6±0.2 degrees two-theta. In some embodiments, crystalline Compound I acetic acid solvate is characterized by an X-ray powder diffractogram having a signal at 21.6±0.2 degrees two-theta. In some embodiments, crystalline Compound I acetic acid solvate is characterized by an X-ray powder diffractogram having a signal at 22.5±0.2 degrees two-theta. In some embodiments, crystalline Compound I acetic acid solvate is characterized by an X-ray powder diffractogram having a signal at 25.0±0.2 degrees two-theta. In some embodiments, crystalline Compound I acetic acid solvate is characterized by an X-ray powder diffractogram having a signal at 25.3±0.2 degrees two-theta. In some embodiments, crystalline Compound I acetic acid solvate is characterized by an X-ray powder diffractogram having a signal at 26.3±0.2 degrees two-theta.

In some embodiments, crystalline Compound I acetic acid solvate is characterized by an X-ray powder diffractogram having signals at one, two, three, four, or five of 5.4±0.2 degrees two-theta, 10.2±0.2 degrees two-theta, 14.2±0.2 degrees two-theta, 15.9±0.2 degrees two-theta, and 20.2±0.2 degrees two-theta.

In some embodiments, crystalline Compound I acetic acid solvate is characterized by an X-ray powder diffractogram having signals at one, two, three, four, five, six, seven, eight, nine, or ten of 5.4±0.2 degrees two-theta, 10.2±0.2 degrees two-theta, 14.2±0.2 degrees two-theta, 15.9±0.2 degrees two-theta, 18.5±0.2 degrees two-theta, 19.5±0.2 degrees two-theta, 19.8±0.2 degrees two-theta, 20.2±0.2 degrees two-theta, 20.6±0.2 degrees two-theta, and 21.6±0.2 degrees two-theta.

In some embodiments, crystalline Compound I acetic acid solvate is characterized by an X-ray powder diffractogram having signals at one, two, three, four, five, six, seven, eight, nine, ten, or more of 5.4±0.2 degrees two-theta, 8.3±0.2 degrees two-theta, 10.2±0.2 degrees two-theta, 10.4±0.2 degrees two-theta, 10.9±0.2 degrees two-theta, 11.7±0.2 degrees two-theta, 13.1±0.2 degrees two-theta, 13.2±0.2 degrees two-theta, 13.8±0.2 degrees two-theta, 14.2±0.2 degrees two-theta, 15.9±0.2 degrees two-theta, 16.7±0.2 degrees two-theta, 17.5±0.2 degrees two-theta, 18.0±0.2 degrees two-theta, 18.5±0.2 degrees two-theta, 18.9±0.2 degrees two-theta, 19.5±0.2 degrees two-theta, 19.8±0.2 degrees two-theta, 20.2±0.2 degrees two-theta, 20.6±0.2 degrees two-theta, 21.6±0.2 degrees two-theta, 22.5±0.2 degrees two-theta, 25.0±0.2 degrees two-theta, 25.3±0.2 degrees two-theta, and 26.3±0.2 degrees two-theta. In some embodiments, crystalline Compound I acetic acid solvate is characterized by an X-ray powder diffractogram having signals at 5.4±0.2 degrees two-theta, 8.3±0.2 degrees two-theta, 10.2±0.2 degrees two-theta, 10.4±0.2 degrees two-theta, 10.9±0.2 degrees two-theta, 11.7±0.2 degrees two-theta, 13.1±0.2 degrees two-theta, 13.2±0.2 degrees two-theta, 13.8±0.2 degrees two-theta, 14.2±0.2 degrees two-theta, 15.9±0.2 degrees two-theta, 16.7±0.2 degrees two-theta, 17.5±0.2 degrees two-theta, 18.0±0.2 degrees two-theta, 18.5±0.2 degrees two-theta, 18.9±0.2 degrees two-theta, 19.5±0.2 degrees two-theta, 19.8±0.2 degrees two-theta, 20.2±0.2 degrees two-theta, 20.6±0.2 degrees two-theta, 21.6±0.2 degrees two-theta, 22.5±0.2 degrees two-theta, 25.0±0.2 degrees two-theta, 25.3±0.2 degrees two-theta, and 26.3±0.2 degrees two-theta.

In some embodiments, crystalline Compound I acetic acid solvate is characterized by an X-ray powder diffractogram substantially similar to FIG. 25.

Another aspect of the invention provides a method of making crystalline Compound I acetic acid solvate. In some embodiments, the method of making crystalline Compound I acetic acid solvate comprises: (i) combining Compound I hemihydrate Form C and acetic acid, and (ii) ball milling at 7500 rpm for 2 cycles of 10 s each with a 60 s pause after each cycle, to yield crystalline Compound I acetic acid solvate.

### H. Crystalline Compound I Heptane Solvate Form B

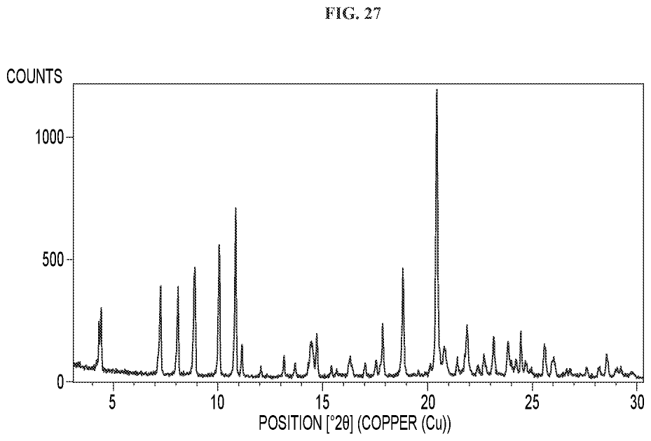

In some embodiments, the invention provides crystalline Compound I heptane solvate Form B. FIG. 27 provides an X-ray powder diffractogram of crystalline Compound I heptane solvate Form B.

In some embodiments, crystalline Compound I heptane solvate Form B is substantially pure. In some embodiments, crystalline Compound I heptane solvate Form B is substantially crystalline. In some embodiments, crystalline Compound I heptane solvate Form B is characterized by an X-ray powder diffractogram generated by an X-ray powder diffraction analysis with an incident beam of Cu Kα radiation.

In some embodiments, crystalline Compound I heptane solvate Form B is characterized by an X-ray powder diffractogram having a signal at 4.4±0.2 degrees two-theta. In some embodiments, crystalline Compound I heptane solvate Form B is characterized by an X-ray powder diffractogram having a signal at 7.3±0.2 degrees two-theta. In some embodiments, crystalline Compound I heptane solvate Form B is characterized by an X-ray powder diffractogram having a signal at 8.9±0.2 degrees two-theta. In some embodiments, crystalline Compound I heptane solvate Form B is characterized by an X-ray powder diffractogram having a signal at 10.9±0.2 degrees two-theta. In some embodiments, crystalline Compound I heptane solvate Form B is characterized by an X-ray powder diffractogram having a signal at 11.1±0.2 degrees two-theta. In some embodiments, crystalline Compound I heptane solvate Form B is characterized by an X-ray powder diffractogram having a signal at 14.4±0.2 degrees two-theta. In some embodiments, crystalline Compound I heptane solvate Form B is characterized by an X-ray powder diffractogram having a signal at 14.7±0.2 degrees two-theta. In some embodiments, crystalline Compound I heptane solvate Form B is characterized by an X-ray powder diffractogram having a signal at 18.8±0.2 degrees two-theta. In some embodiments, crystalline Compound I heptane solvate Form B is characterized by an X-ray powder diffractogram having a signal at 21.9±0.2 degrees two-theta. In some embodiments, crystalline Compound I heptane solvate Form B is characterized by an X-ray powder diffractogram having a signal at 23.2±0.2 degrees two-theta. In some embodiments, crystalline Compound I heptane solvate Form B is characterized by an X-ray powder diffractogram having a signal at 23.8±0.2 degrees two-theta. In some embodiments, crystalline Compound I heptane solvate Form B is characterized by an X-ray powder diffractogram having a signal at 24.5±0.2 degrees two-theta. In some embodiments, crystalline Compound I heptane solvate Form B is characterized by an X-ray powder diffractogram having a signal at 25.6±0.2 degrees two-theta.

In some embodiments, crystalline Compound I heptane solvate Form B is characterized by an X-ray powder diffractogram having a signal at one, two, three, four, five, six, seven, eight, nine, ten, or more of 4.4±0.2 degrees two-theta, 7.3±0.2 degrees two-theta, 8.9±0.2 degrees two-theta, 10.9±0.2 degrees two-theta, 11.1±0.2 degrees two-theta, 14.4±0.2 degrees two-theta, 14.7±0.2 degrees two-theta, 18.8±0.2 degrees two-theta, 21.9±0.2 degrees two-theta, 23.2±0.2 degrees two-theta, 23.8±0.2 degrees two-theta, 24.5±0.2 degrees two-theta, and 25.6±0.2 degrees two-theta.

In some embodiments, crystalline Compound I heptane solvate Form B is characterized by an X-ray powder diffractogram having signals at (a) one, two, three, four, five, six, seven, eight, nine, ten, or more of 4.4±0.2 degrees two-theta, 7.3±0.2 degrees two-theta, 8.9±0.2 degrees two-theta, 10.9±0.2 degrees two-theta, 11.1±0.2 degrees two-theta, 14.4±0.2 degrees two-theta, 14.7±0.2 degrees two-theta, 18.8±0.2 degrees two-theta, 21.9±0.2 degrees two-theta, 23.2±0.2 degrees two-theta, 23.8±0.2 degrees two-theta, 24.5±0.2 degrees two-theta, and 25.6±0.2 degrees two-theta, and (b) one, two, three, or four of 8.1±0.2 degrees two-theta, 10.1±0.2 degrees two-theta, 17.9±0.2 degrees two-theta, and 20.4±0.2 degrees two-theta.

In some embodiments, crystalline Compound I heptane solvate Form B is characterized by an X-ray powder diffractogram having signals at four, five, six, seven, eight, nine, ten, or more of 4.4±0.2 degrees two-theta, 7.3±0.2 degrees two-theta, 8.1±0.2 degrees two-theta, 8.9±0.2 degrees two-theta, 10.1±0.2 degrees two-theta, 10.9±0.2 degrees two-theta, 11.1±0.2 degrees two-theta, 14.4±0.2 degrees two-theta, 14.7±0.2 degrees two-theta, 17.9±0.2 degrees two-theta, 18.8±0.2 degrees two-theta, 20.4±0.2 degrees two-theta, 21.9±0.2 degrees two-theta, 23.2±0.2 degrees two-theta, 23.8±0.2 degrees two-theta, 24.5±0.2 degrees two-theta, and 25.6±0.2 degrees two-theta.

In some embodiments, crystalline Compound I heptane solvate Form B is characterized by an X-ray powder diffractogram having signals at 4.4±0.2 degrees two-theta, 7.3±0.2 degrees two-theta, 8.1±0.2 degrees two-theta, 8.9±0.2 degrees two-theta, 10.1±0.2 degrees two-theta, 10.9±0.2 degrees two-theta, 11.1±0.2 degrees two-theta, 14.4±0.2 degrees two-theta, 14.7±0.2 degrees two-theta, 17.9±0.2 degrees two-theta, 18.8±0.2 degrees two-theta, 20.4±0.2 degrees two-theta, 21.9±0.2 degrees two-theta, 23.2±0.2 degrees two-theta, 23.8±0.2 degrees two-theta, 24.5±0.2 degrees two-theta, and 25.6±0.2 degrees two-theta.

In some embodiments, crystalline Compound I heptane solvate Form B is characterized by an X-ray powder diffractogram substantially similar to FIG. 27.

In some embodiments, crystalline Compound I heptane solvate Form B is characterized as having a ¹³C SSNMR spectrum with a peak at 137.5±0.2 ppm. In some embodiments, crystalline Compound I heptane solvate Form B is characterized as having a ¹³C SSNMR spectrum with a peak at 117.4±0.2 ppm. In some embodiments, crystalline Compound I heptane solvate Form B is characterized as having a ¹³C SSNMR spectrum with a peak at 126.3±0.2 ppm. In some embodiments, crystalline Compound I heptane solvate Form B is characterized as having a ¹³C SSNMR spectrum with a peak at 75.5±0.2 ppm. In some embodiments, crystalline Compound I heptane solvate Form B is characterized as having a ¹³C SSNMR spectrum with a peak at 34.2±0.2 ppm.

In some embodiments, crystalline Compound I heptane solvate Form B is characterized as having a ¹³C SSNMR spectrum with one, two, three, four, or five peaks selected from 137.5±0.2 ppm, 126.3±0.2 ppm, 117.4±0.2 ppm, 75.5±0.2 ppm, and 34.2±0.2 ppm.

In some embodiments, crystalline Compound I heptane solvate Form B is characterized as having a ¹³C SSNMR spectrum with (a) one, two, three, four, or five peaks selected from 137.5±0.2 ppm, 126.3±0.2 ppm, 117.4±0.2 ppm, 75.5±0.2 ppm, and 34.2±0.2 ppm, and (b) one, two, three, four, five, six, seven, eight, nine, ten, or more peaks selected from 164.4±0.2 ppm, 163.0±0.2 ppm, 151.0±0.2 ppm, 139.5±0.2 ppm, 120.8±0.2 ppm, 120.0±0.2 ppm, 74.7±0.2 ppm, 74.1±0.2 ppm, 73.0±0.2 ppm, 31.1±0.2 ppm, 28.2±0.2 ppm, 22.4±0.2 ppm, 20.8±0.2 ppm, 19.5±0.2 ppm, and 13.8±0.2 ppm.

In some embodiments, crystalline Compound I heptane solvate Form B is characterized as having a ¹³C SSNMR spectrum with twelve or more peaks selected from 164.4±0.2 ppm, 163.0±0.2 ppm, 151.0±0.2 ppm, 139.5±0.2 ppm, 137.5±0.2 ppm, 126.3±0.2 ppm, 120.8±0.2 ppm, 120.0±0.2 ppm, 117.4±0.2 ppm, 75.5±0.2 ppm, 74.7±0.2 ppm, 74.1±0.2 ppm, 73.0±0.2 ppm, 34.2±0.2 ppm, 31.1±0.2 ppm, 28.2±0.2 ppm, 22.4±0.2 ppm, 20.8±0.2 ppm, 19.5±0.2 ppm, and 13.8±0.2 ppm.

In some embodiments, crystalline Compound I heptane solvate Form B is characterized as having a ¹³C SSNMR spectrum with peaks at 164.4±0.2 ppm, 163.0±0.2 ppm, 151.0±0.2 ppm, 139.5±0.2 ppm, 137.5±0.2 ppm, 126.3±0.2 ppm, 120.8±0.2 ppm, 120.0±0.2 ppm, 117.4±0.2 ppm, 75.5±0.2 ppm, 74.7±0.2 ppm, 74.1±0.2 ppm, 73.0±0.2 ppm, 34.2±0.2 ppm, 31.1±0.2 ppm, 28.2±0.2 ppm, 22.4±0.2 ppm, 20.8±0.2 ppm, 19.5±0.2 ppm, and 13.8±0.2 ppm.

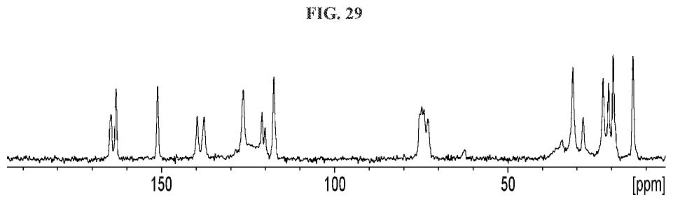

In some embodiments, crystalline Compound I heptane solvate Form B is characterized by a ¹³C SSNMR spectrum substantially similar to FIG. 29.

In some embodiments, crystalline Compound I heptane solvate Form B is characterized as having a ¹⁹F SSNMR spectrum with a peak at −78.4±0.2 ppm. In some embodiments, crystalline Compound I heptane solvate Form B is characterized as having a ¹⁹F SSNMR spectrum with a peak at −64.2±0.2 ppm. In some embodiments, crystalline Compound I heptane solvate Form B is characterized as having a ¹⁹F SSNMR spectrum with (a) one or two peaks selected from −78.4±0.2 ppm and −64.2±0.2 ppm, and (b) one or two peaks selected from −63.4±0.2 ppm and −77.4±0.2 ppm.

In some embodiments, crystalline Compound I heptane solvate Form B is characterized as having a ¹⁹F SSNMR spectrum with three or four peaks selected from −78.4±0.2 ppm, −77.4±0.2 ppm, −64.2±0.2 ppm, and −63.4±0.2 ppm.

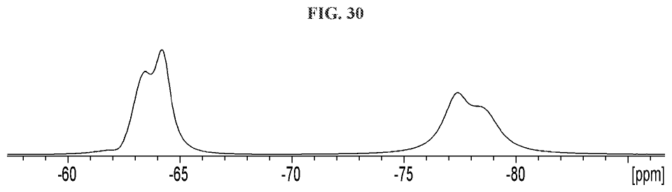

In some embodiments, crystalline Compound I heptane solvate Form B is characterized by a ¹⁹F SSNMR spectrum substantially similar to FIG. 30.

Another aspect of the invention provides a method of making crystalline Compound I heptane solvate Form B. In some embodiments, the method of making crystalline Compound I heptane solvate Form B comprises: (i) adding 1-butanol/heptane (75 v % heptane) to crystalline Compound I neat Form D and (ii) shaking the mixture at 25° C. for 2 days to yield crystalline Compound I heptane solvate Form B.

### I. Crystalline Compound I Heptane Solvate Form C

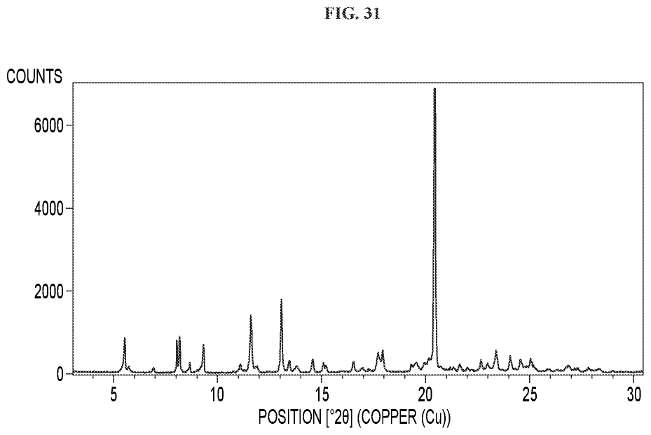

In some embodiments, the invention provides crystalline Compound I heptane solvate Form C. FIG. 31 provides an X-ray powder diffractogram of crystalline Compound I heptane solvate Form C.

In some embodiments, crystalline Compound I heptane solvate Form C is substantially pure. In some embodiments, crystalline Compound I heptane solvate Form C is substantially crystalline. In some embodiments, crystalline Compound I heptane solvate Form C is characterized by an X-ray powder diffractogram generated by an X-ray powder diffraction analysis with an incident beam of Cu Kα radiation.

In some embodiments, crystalline Compound I heptane solvate Form C is characterized by an X-ray powder diffractogram having a signal at 9.3±0.2 degrees two-theta. In some embodiments, crystalline Compound I heptane solvate Form C is characterized by an X-ray powder diffractogram having a signal at 13.1±0.2 degrees two-theta. In some embodiments, crystalline Compound I heptane solvate Form C is characterized by an X-ray powder diffractogram having a signal at 32.3±0.2 degrees two-theta.

In some embodiments, crystalline Compound I heptane solvate Form C is characterized by an X-ray powder diffractogram having a signal at one or two of 9.3±0.2 degrees two-theta, 13.1±0.2 degrees two-theta, and 32.3±0.2 degrees two-theta. In some embodiments, crystalline Compound I heptane solvate Form C is characterized by an X-ray powder diffractogram having signals at 9.3±0.2 degrees two-theta, 13.1±0.2 degrees two-theta, and 32.3±0.2 degrees two-theta.

In some embodiments, crystalline Compound I heptane solvate Form C is characterized by an X-ray powder diffractogram having signals at (a) one, two, or three of 9.3±0.2 degrees two-theta, 13.1±0.2 degrees two-theta, and 32.3±0.2 degrees two-theta, and (b) one, two, three, four, or five of 5.5±0.2 degrees two-theta, 8.0±0.2 degrees two-theta, 8.2±0.2 degrees two-theta, 11.6±0.2 degrees two-theta, and 20.4±0.2 degrees two-theta.

In some embodiments, crystalline Compound I heptane solvate Form C is characterized by an X-ray powder diffractogram having signals at five or six of 5.5±0.2 degrees two-theta, 8.0±0.2 degrees two-theta, 8.2±0.2 degrees two-theta, 9.3±0.2 degrees two-theta, 11.6±0.2 degrees two-theta, 13.1±0.2 degrees two-theta, 20.4±0.2 degrees two-theta, and 32.3±0.2 degrees two-theta.

In some embodiments, crystalline Compound I heptane solvate Form C is characterized by an X-ray powder diffractogram having signals at 5.5±0.2 degrees two-theta, 8.0±0.2 degrees two-theta, 8.2±0.2 degrees two-theta, 9.3±0.2 degrees two-theta, 11.6±0.2 degrees two-theta, 13.1±0.2 degrees two-theta, 20.4±0.2 degrees two-theta, and 32.3±0.2 degrees two-theta.

In some embodiments, crystalline Compound I heptane solvate Form C is characterized by an X-ray powder diffractogram substantially similar to FIG. 31.

In some embodiments, crystalline Compound I heptane solvate Form C is characterized as having a ¹³C SSNMR spectrum with a peak at 126.9±0.2 ppm. In some embodiments, crystalline Compound I heptane solvate Form C is characterized as having a ¹³C SSNMR spectrum with a peak at 124.1±0.2 ppm. In some embodiments, crystalline Compound I heptane solvate Form C is characterized as having a ¹³C SSNMR spectrum with a peak at 121.5±0.2 ppm. In some embodiments, crystalline Compound I heptane solvate Form C is characterized as having a ¹³C SSNMR spectrum with a peak at 118.8±0.2 ppm. In some embodiments, crystalline Compound I heptane solvate Form C is characterized as having a ¹³C SSNMR spectrum with a peak at 71.5±0.2 ppm. In some embodiments, crystalline Compound I heptane solvate Form C is characterized as having a ¹³C SSNMR spectrum with a peak at 36.1±0.2 ppm. In some embodiments, crystalline Compound I heptane solvate Form C is characterized as having a ¹³C SSNMR spectrum with a peak at 24.3±0.2 ppm. In some embodiments, crystalline Compound I heptane solvate Form C is characterized as having a ¹³C SSNMR spectrum with a peak at 14.2±0.2 ppm.

In some embodiments, crystalline Compound I heptane solvate Form C is characterized as having a ¹³C SSNMR spectrum with one, two, three, four, five, six, seven, or eight peaks selected from 126.9±0.2 ppm, 124.1±0.2 ppm, 121.5±0.2 ppm, 118.8±0.2 ppm, 71.5±0.2 ppm, 36.1±0.2 ppm, 24.3±0.2 ppm, and 14.2±0.2 ppm.

In some embodiments, crystalline Compound I heptane solvate Form C is characterized as having a ¹³C SSNMR spectrum with (a) one, two, three, four, five, six, seven, or eight peaks selected from 126.9±0.2 ppm, 124.1±0.2 ppm, 121.5±0.2 ppm, 118.8±0.2 ppm, 71.5±0.2 ppm, 36.1±0.2 ppm, 24.3±0.2 ppm, and 14.2±0.2 ppm, and (b) one, two, three, four, five, six, seven, eight, nine, ten, or more peaks selected from 164.4±0.2 ppm, 163.6±0.2 ppm, 163.1±0.2 ppm, 151.1±0.2 ppm, 139.4±0.2 ppm, 128.3±0.2 ppm, 117.9±0.2 ppm, 76.1±0.2 ppm, 73.6±0.2 ppm, 37.0±0.2 ppm, 33.6±0.2 ppm, 30.9±0.2 ppm, 27.9±0.2 ppm, 23.3±0.2 ppm, 21.0±0.2 ppm, 20.0±0.2 ppm, and 18.1±0.2 ppm.

In some embodiments, crystalline Compound I heptane solvate Form C is characterized as having a ¹³C SSNMR spectrum with seventeen or more peaks selected from 164.4±0.2 ppm, 163.6±0.2 ppm, 163.1±0.2 ppm, 151.1±0.2 ppm, 139.4±0.2 ppm, 128.3±0.2 ppm, 126.9±0.2 ppm, 124.1±0.2 ppm, 121.5±0.2 ppm, 118.8±0.2 ppm, 117.9±0.2 ppm, 76.1±0.2 ppm, 73.6±0.2 ppm, 71.5±0.2 ppm, 37.0±0.2 ppm, 36.1±0.2 ppm, 33.6±0.2 ppm, 30.9±0.2 ppm, 27.9±0.2 ppm, 24.3±0.2 ppm, 23.3±0.2 ppm, 21.0±0.2 ppm, 20.0±0.2 ppm, 18.1±0.2 ppm, and 14.2±0.2 ppm.

In some embodiments, crystalline Compound I heptane solvate Form C is characterized as having a ¹³C SSNMR spectrum with peaks at 164.4±0.2 ppm, 163.6±0.2 ppm, 163.1±0.2 ppm, 151.1±0.2 ppm, 139.4±0.2 ppm, 128.3±0.2 ppm, 126.9±0.2 ppm, 124.1±0.2 ppm, 121.5±0.2 ppm, 118.8±0.2 ppm, 117.9±0.2 ppm, 76.1±0.2 ppm, 73.6±0.2 ppm, 71.5±0.2 ppm, 37.0±0.2 ppm, 36.1±0.2 ppm, 33.6±0.2 ppm, 30.9±0.2 ppm, 27.9±0.2 ppm, 24.3±0.2 ppm, 23.3±0.2 ppm, 21.0±0.2 ppm, 20.0±0.2 ppm, 18.1±0.2 ppm, and 14.2±0.2 ppm.

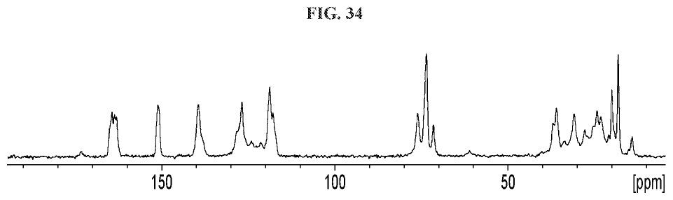

In some embodiments, crystalline Compound I heptane solvate Form C is characterized by a ¹³C SSNMR spectrum substantially similar to FIG. 34.

Another aspect of the invention provides a method of making crystalline Compound I heptane solvate Form C. In some embodiments, the method of making crystalline Compound I heptane solvate Form C comprises: (i) adding ethyl acetate/heptane (25 v % heptane) to crystalline Compound I neat Form D and (ii) shaking at 25° C. for 2 days to yield crystalline Compound I heptane solvate Form C.

### J. Crystalline Compound I Octane Solvate

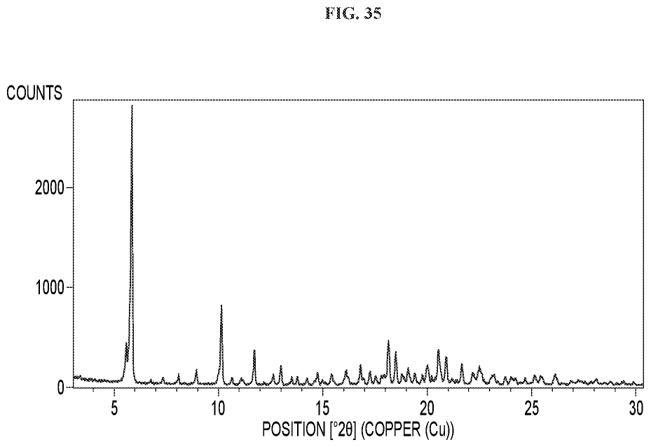

In some embodiments, the invention provides crystalline Compound I octane solvate. FIG. 35 provides an X-ray powder diffractogram of crystalline Compound I octane solvate.

In some embodiments, crystalline Compound I octane solvate is substantially pure. In some embodiments, crystalline Compound I octane solvate is substantially crystalline. In some embodiments, crystalline Compound I octane solvate is characterized by an X-ray powder diffractogram generated by an X-ray powder diffraction analysis with an incident beam of Cu Kα radiation.

In some embodiments, crystalline Compound I octane solvate is characterized by an X-ray powder diffractogram having a signal at 5.6±0.2 degrees two-theta. In some embodiments, crystalline Compound I octane solvate is characterized by an X-ray powder diffractogram having a signal at 5.9±0.2 degrees two-theta. In some embodiments, crystalline Compound I octane solvate is characterized by an X-ray powder diffractogram having a signal at 10.2±0.2 degrees two-theta. In some embodiments, crystalline Compound I octane solvate is characterized by an X-ray powder diffractogram having a signal at 11.7±0.2 degrees two-theta. In some embodiments, crystalline Compound I octane solvate is characterized by an X-ray powder diffractogram having a signal at 18.2±0.2 degrees two-theta. In some embodiments, crystalline Compound I octane solvate is characterized by an X-ray powder diffractogram having a signal at 18.5±0.2 degrees two-theta. In some embodiments, crystalline Compound I octane solvate is characterized by an X-ray powder diffractogram having a signal at 20.5±0.2 degrees two-theta.

In some embodiments, crystalline Compound I octane solvate is characterized by an X-ray powder diffractogram having signals at one, two, three, four, or five of 5.6±0.2 degrees two-theta, 5.9±0.2 degrees two-theta, 10.2±0.2 degrees two-theta, 11.7±0.2 degrees two-theta, and 18.2±0.2 degrees two-theta.

In some embodiments, crystalline Compound I octane solvate is characterized by an X-ray powder diffractogram having signals at one, two, three, four, five, six, or seven of 5.6±0.2 degrees two-theta, 5.9±0.2 degrees two-theta, 10.2±0.2 degrees two-theta, 11.7±0.2 degrees two-theta, 18.2±0.2 degrees two-theta, 18.5±0.2 degrees two-theta, and 20.5±0.2 degrees two-theta.

In some embodiments, crystalline Compound I octane solvate is characterized by an X-ray powder diffractogram substantially similar to FIG. 35.

In some embodiments, crystalline Compound I octane solvate is characterized as having a ¹³C SSNMR spectrum with a peak at 166.3±0.2 ppm. In some embodiments, crystalline Compound I octane solvate is characterized as having a ¹³C SSNMR spectrum with a peak at 164.6±0.2 ppm. In some embodiments, crystalline Compound I octane solvate is characterized as having a ¹³C SSNMR spectrum with a peak at 164.1±0.2 ppm. In some embodiments, crystalline Compound I octane solvate is characterized as having a ¹³C SSNMR spectrum with a peak at 153.8±0.2 ppm. In some embodiments, crystalline Compound I octane solvate is characterized as having a ¹³C SSNMR spectrum with a peak at 152.2±0.2 ppm. In some embodiments, crystalline Compound I octane solvate is characterized as having a ¹³C SSNMR spectrum with a peak at 151.7±0.2 ppm. In some embodiments, crystalline Compound I octane solvate is characterized as having a ¹³C SSNMR spectrum with a peak at 140.4±0.2 ppm. In some embodiments, crystalline Compound I octane solvate is characterized as having a ¹³C SSNMR spectrum with a peak at 137.6±0.2 ppm. In some embodiments, crystalline Compound I octane solvate is characterized as having a ¹³C SSNMR spectrum with a peak at 135.3±0.2 ppm. In some embodiments, crystalline Compound I octane solvate is characterized as having a ¹³C SSNMR spectrum with a peak at 134.8±0.2 ppm. In some embodiments, crystalline Compound I octane solvate is characterized as having a ¹³C SSNMR spectrum with a peak at 131.1±0.2 ppm. In some embodiments, crystalline Compound I octane solvate is characterized as having a ¹³C SSNMR spectrum with a peak at 130.2±0.2 ppm. In some embodiments, crystalline Compound I octane solvate is characterized as having a ¹³C SSNMR spectrum with a peak at 127.3±0.2 ppm. In some embodiments, crystalline Compound I octane solvate is characterized as having a ¹³C SSNMR spectrum with a peak at 125.5±0.2 ppm. In some embodiments, crystalline Compound I octane solvate is characterized as having a ¹³C SSNMR spectrum with a peak at 122.7±0.2 ppm. In some embodiments, crystalline Compound I octane solvate is characterized as having a ¹³C SSNMR spectrum with a peak at 120.8±0.2 ppm. In some embodiments, crystalline Compound I octane solvate is characterized as having a ¹³C SSNMR spectrum with a peak at 120.1±0.2 ppm. In some embodiments, crystalline Compound I octane solvate is characterized as having a ¹³C SSNMR spectrum with a peak at 118.1±0.2 ppm. In some embodiments, crystalline Compound I octane solvate is characterized as having a ¹³C SSNMR spectrum with a peak at 75.7±0.2 ppm. In some embodiments, crystalline Compound I octane solvate is characterized as having a ¹³C SSNMR spectrum with a peak at 74.4±0.2 ppm. In some embodiments, crystalline Compound I octane solvate is characterized as having a ¹³C SSNMR spectrum with a peak at 73.8±0.2 ppm. In some embodiments, crystalline Compound I octane solvate is characterized as having a ¹³C SSNMR spectrum with a peak at 40.2±0.2 ppm. In some embodiments, crystalline Compound I octane solvate is characterized as having a ¹³C SSNMR spectrum with a peak at 37.5±0.2 ppm. In some embodiments, crystalline Compound I octane solvate is characterized as having a ¹³C SSNMR spectrum with a peak at 36.1±0.2 ppm. In some embodiments, crystalline Compound I octane solvate is characterized as having a ¹³C SSNMR spectrum with a peak at 32.0±0.2 ppm. In some embodiments, crystalline Compound I octane solvate is characterized as having a ¹³C SSNMR spectrum with a peak at 29.9±0.2 ppm. In some embodiments, crystalline Compound I octane solvate is characterized as having a ¹³C SSNMR spectrum with a peak at 28.5±0.2 ppm. In some embodiments, crystalline Compound I octane solvate is characterized as having a ¹³C SSNMR spectrum with a peak at 27.0±0.2 ppm. In some embodiments, crystalline Compound I octane solvate is characterized as having a ¹³C SSNMR spectrum with a peak at 25.1±0.2 ppm. In some embodiments, crystalline Compound I octane solvate is characterized as having a ¹³C SSNMR spectrum with a peak at 22.4±0.2 ppm. In some embodiments, crystalline Compound I octane solvate is characterized as having a ¹³C SSNMR spectrum with a peak at 20.0±0.2 ppm. In some embodiments, crystalline Compound I octane solvate is characterized as having a ¹³C SSNMR spectrum with a peak at 17.7±0.2 ppm. In some embodiments, crystalline Compound I octane solvate is characterized as having a ¹³C SSNMR spectrum with a peak at 14.1±0.2 ppm. In some embodiments, crystalline Compound I octane solvate is characterized as having a ¹³C SSNMR spectrum with a peak at 13.5±0.2 ppm. In some embodiments, crystalline Compound I octane solvate is characterized as having a ¹³C SSNMR spectrum with a peak at 12.6±0.2 ppm.

In some embodiments, crystalline Compound I octane solvate is characterized as having a ¹³C SSNMR spectrum with one, two, three, four, five, six, seven, eight, nine, ten, or more peaks selected from 166.3±0.2 ppm, 164.6±0.2 ppm, 164.1±0.2 ppm, 153.8±0.2 ppm, 152.2±0.2 ppm, 151.7±0.2 ppm, 140.4±0.2 ppm, 137.6±0.2 ppm, 135.3±0.2 ppm, 134.8±0.2 ppm, 131.1±0.2 ppm, 130.2±0.2 ppm, 127.3±0.2 ppm, 125.5±0.2 ppm, 122.7±0.2 ppm, 120.8±0.2 ppm, 120.1±0.2 ppm, 118.1±0.2 ppm, 75.7±0.2 ppm, 74.4±0.2 ppm, 73.8±0.2 ppm, 40.2±0.2 ppm, 37.5±0.2 ppm, 36.1±0.2 ppm, 32.0±0.2 ppm, 29.9±0.2 ppm, 28.5±0.2 ppm, 27.0±0.2 ppm, 25.1±0.2 ppm, 22.4±0.2 ppm, 20.0±0.2 ppm, 17.7±0.2 ppm, 14.1±0.2 ppm, 13.5±0.2 ppm, and 12.6±0.2 ppm. In some embodiments, crystalline Compound I octane solvate is characterized as having a ¹³C SSNMR spectrum with peaks at 166.3±0.2 ppm, 164.6±0.2 ppm, 164.1±0.2 ppm, 153.8±0.2 ppm, 152.2±0.2 ppm, 151.7±0.2 ppm, 140.4±0.2 ppm, 137.6±0.2 ppm, 135.3±0.2 ppm, 134.8±0.2 ppm, 131.1±0.2 ppm, 130.2±0.2 ppm, 127.3±0.2 ppm, 125.5±0.2 ppm, 122.7±0.2 ppm, 120.8±0.2 ppm, 120.1±0.2 ppm, 118.1±0.2 ppm, 75.7±0.2 ppm, 74.4±0.2 ppm, 73.8±0.2 ppm, 40.2±0.2 ppm, 37.5±0.2 ppm, 36.1±0.2 ppm, 32.0±0.2 ppm, 29.9±0.2 ppm, 28.5±0.2 ppm, 27.0±0.2 ppm, 25.1±0.2 ppm, 22.4±0.2 ppm, 20.0±0.2 ppm, 17.7±0.2 ppm, 14.1±0.2 ppm, 13.5±0.2 ppm, and 12.6±0.2 ppm.

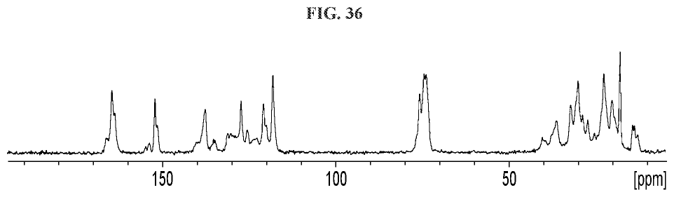

In some embodiments, crystalline Compound I octane solvate is characterized by a ¹³C SSNMR spectrum substantially similar to FIG. 36.

In some embodiments, crystalline Compound I octane solvate is characterized as having a ¹⁹F SSNMR spectrum with a peak at −62.5±0.2 ppm. In some embodiments, crystalline Compound I octane solvate is characterized as having a ¹⁹F SSNMR spectrum with a peak at −65.0±0.2 ppm. In some embodiments, crystalline Compound I octane solvate is characterized as having a ¹⁹F SSNMR spectrum with a peak at −65.6±0.2 ppm. In some embodiments, crystalline Compound I octane solvate is characterized as having a ¹⁹F SSNMR spectrum with a peak at −66.2±0.2 ppm. In some embodiments, crystalline Compound I octane solvate is characterized as having a ¹⁹F SSNMR spectrum with a peak at −67.1±0.2 ppm. In some embodiments, crystalline Compound I octane solvate is characterized as having a ¹⁹F SSNMR spectrum with a peak at −75.1±0.2 ppm. In some embodiments, crystalline Compound I octane solvate is characterized as having a ¹⁹F SSNMR spectrum with a peak at −76.5±0.2 ppm. In some embodiments, crystalline Compound I octane solvate is characterized as having a ¹⁹F SSNMR spectrum with a peak at −77.2±0.2 ppm.

In some embodiments, crystalline Compound I octane solvate is characterized as having a ¹⁹F SSNMR spectrum with one, two, three, four, five, six, seven, or more peaks selected from −62.5±0.2 ppm, −65.0±0.2 ppm, −65.6±0.2 ppm, −66.2±0.2 ppm, −67.1±0.2 ppm, −75.1±0.2 ppm, −76.5±0.2 ppm, and −77.2±0.2 ppm. In some embodiments, crystalline Compound I octane solvate is characterized as having a ¹⁹F SSNMR spectrum with peaks at −62.5±0.2 ppm, −65.0±0.2 ppm, −65.6±0.2 ppm, −66.2±0.2 ppm, −67.1±0.2 ppm, −75.1±0.2 ppm, −76.5±0.2 ppm, and −77.2±0.2 ppm.

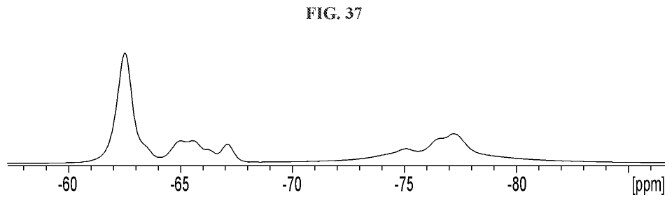

In some embodiments, crystalline Compound I octane solvate is characterized by a ¹⁹F SSNMR spectrum substantially similar to FIG. 37.

Another aspect of the invention provides a method of making crystalline Compound I octane solvate. In some embodiments, the method of making crystalline Compound I octane solvate comprises shaking crystalline Compound I hemihydrate Form C in octane at 35° C. for about one week to yield crystalline Compound I octane solvate.

### K. Crystalline Compound I Cyclohexane Solvate Form A

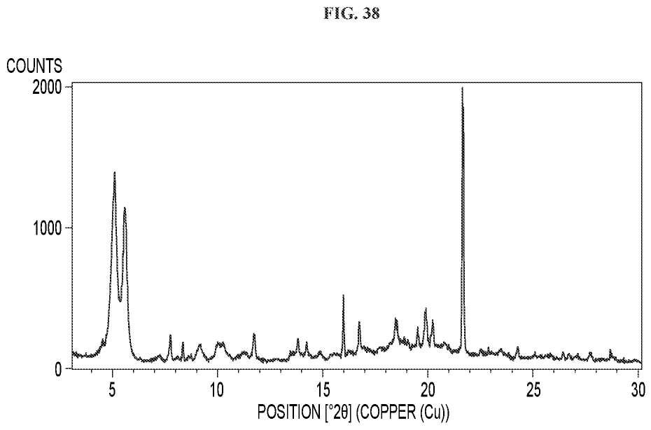

In some embodiments, the invention provides crystalline Compound I cyclohexane solvate Form A. FIG. 38 provides an X-ray powder diffractogram of crystalline Compound I cyclohexane solvate Form A.

In some embodiments, crystalline Compound I cyclohexane solvate Form A is substantially pure. In some embodiments, crystalline Compound I cyclohexane solvate Form A is substantially crystalline. In some embodiments, crystalline Compound I cyclohexane solvate Form A is characterized by an X-ray powder diffractogram generated by an X-ray powder diffraction analysis with an incident beam of Cu Ku radiation.

In some embodiments, crystalline Compound I cyclohexane solvate Form A is characterized by an X-ray powder diffractogram having a signal at 5.1±0.2 degrees two-theta. In some embodiments, crystalline Compound I cyclohexane solvate Form A is characterized by an X-ray powder diffractogram having a signal at 16.0±0.2 degrees two-theta. In some embodiments, crystalline Compound I cyclohexane solvate Form A is characterized by an X-ray powder diffractogram having a signal at 33.6±0.2 degrees two-theta.

In some embodiments, crystalline Compound I cyclohexane solvate Form A is characterized by an X-ray powder diffractogram having a signal at one or two of 5.1±0.2 degrees two-theta, 16.0±0.2 degrees two-theta, and 33.6±0.2 degrees two-theta. In some embodiments, crystalline Compound I cyclohexane solvate Form A is characterized by an X-ray powder diffractogram having signals at 5.1±0.2 degrees two-theta, 16.0±0.2 degrees two-theta, and 33.6±0.2 degrees two-theta.

In some embodiments, crystalline Compound I cyclohexane solvate Form A is characterized by an X-ray powder diffractogram having signals at (a) one, two, or three of 5.1±0.2 degrees two-theta, 16.0±0.2 degrees two-theta, and 33.6±0.2 degrees two-theta, and (b) one, two, three, four, or five of 5.6±0.2 degrees two-theta, 16.7±0.2 degrees two-theta, 18.5±0.2 degrees two-theta, 19.9±0.2 degrees two-theta, and 21.6±0.2 degrees two-theta.

In some embodiments, crystalline Compound I cyclohexane solvate Form A is characterized by an X-ray powder diffractogram having signals at five, six, seven, or more of 5.1±0.2 degrees two-theta, 5.6±0.2 degrees two-theta, 16.0±0.2 degrees two-theta, 16.7±0.2 degrees two-theta, 18.5±0.2 degrees two-theta, 19.9±0.2 degrees two-theta, 21.6±0.2 degrees two-theta, and 33.6±0.2 degrees two-theta.

In some embodiments, crystalline Compound I cyclohexane solvate Form A is characterized by an X-ray powder diffractogram having signals at 5.1±0.2 degrees two-theta, 5.6±0.2 degrees two-theta, 16.0±0.2 degrees two-theta, 16.7±0.2 degrees two-theta, 18.5±0.2 degrees two-theta, 19.9±0.2 degrees two-theta, 21.6±0.2 degrees two-theta, and 33.6±0.2 degrees two-theta.

In some embodiments, crystalline Compound I cyclohexane solvate Form A is characterized by an X-ray powder diffractogram substantially similar to FIG. 38.

In some embodiments, crystalline Compound I cyclohexane solvate Form A is characterized as having a ¹³C SSNMR spectrum with a peak at 166.6±0.2 ppm. In some embodiments, crystalline Compound I cyclohexane solvate Form A is characterized as having a ¹³C SSNMR spectrum with a peak at 152.1±0.2 ppm. In some embodiments, crystalline Compound I cyclohexane solvate Form A is characterized as having a ¹³C SSNMR spectrum with a peak at 150.8±0.2 ppm. In some embodiments, crystalline Compound I cyclohexane solvate Form A is characterized as having a ¹³C SSNMR spectrum with a peak at 140.4±0.2 ppm. In some embodiments, crystalline Compound I cyclohexane solvate Form A is characterized as having a ¹³C SSNMR spectrum with a peak at 137.6±0.2 ppm. In some embodiments, crystalline Compound I cyclohexane solvate Form A is characterized as having a ¹³C SSNMR spectrum with a peak at 135.4±0.2 ppm. In some embodiments, crystalline Compound I cyclohexane solvate Form A is characterized as having a ¹³C SSNMR spectrum with a peak at 127.3±0.2 ppm. In some embodiments, crystalline Compound I cyclohexane solvate Form A is characterized as having a ¹³C SSNMR spectrum with a peak at 125.5±0.2 ppm. In some embodiments, crystalline Compound I cyclohexane solvate Form A is characterized as having a ¹³C SSNMR spectrum with a peak at 123.4±0.2 ppm. In some embodiments, crystalline Compound I cyclohexane solvate Form A is characterized as having a ¹³C SSNMR spectrum with a peak at 119.7±0.2 ppm. In some embodiments, crystalline Compound I cyclohexane solvate Form A is characterized as having a ¹³C SSNMR spectrum with a peak at 74.3±0.2 ppm. In some embodiments, crystalline Compound I cyclohexane solvate Form A is characterized as having a ¹³C SSNMR spectrum with a peak at 37.4±0.2 ppm. In some embodiments, crystalline Compound I cyclohexane solvate Form A is characterized as having a ¹³C SSNMR spectrum with a peak at 36.2±0.2 ppm. In some embodiments, crystalline Compound I cyclohexane solvate Form A is characterized as having a ¹³C SSNMR spectrum with a peak at 30.6±0.2 ppm. In some embodiments, crystalline Compound I cyclohexane solvate Form A is characterized as having a ¹³C SSNMR spectrum with a peak at 27.4±0.2 ppm. In some embodiments, crystalline Compound I cyclohexane solvate Form A is characterized as having a ¹³C SSNMR spectrum with a peak at 17.7±0.2 ppm.

In some embodiments, crystalline Compound I cyclohexane solvate Form A is characterized as having a ¹³C SSNMR spectrum with one, two, three, four, five, six, seven, eight, nine, ten, or more peaks selected from 166.6±0.2 ppm, 152.1±0.2 ppm, 150.8±0.2 ppm, 140.4±0.2 ppm, 137.6±0.2 ppm, 135.4±0.2 ppm, 127.3±0.2 ppm, 125.5±0.2 ppm, 123.4±0.2 ppm, 119.7±0.2 ppm, 74.3±0.2 ppm, 37.4±0.2 ppm, 36.2±0.2 ppm, 30.6±0.2 ppm, 27.4±0.2 ppm, and 17.7±0.2 ppm. In some embodiments, crystalline Compound I cyclohexane solvate Form A is characterized as having a ¹³C SSNMR spectrum with peaks at 166.6±0.2 ppm, 152.1±0.2 ppm, 150.8±0.2 ppm, 140.4±0.2 ppm, 137.6±0.2 ppm, 135.4±0.2 ppm, 127.3±0.2 ppm, 125.5±0.2 ppm, 123.4±0.2 ppm, 119.7±0.2 ppm, 74.3±0.2 ppm, 37.4±0.2 ppm, 36.2±0.2 ppm, 30.6±0.2 ppm, 27.4±0.2 ppm, and 17.7±0.2 ppm.

In some embodiments, crystalline Compound I cyclohexane solvate Form A is characterized as having a ¹³C SSNMR spectrum with (a) one, two, three, four, five, six, seven, eight, nine, ten, or more peaks selected from 166.6±0.2 ppm, 152.1±0.2 ppm, 150.8±0.2 ppm, 140.4±0.2 ppm, 137.6±0.2 ppm, 135.4±0.2 ppm, 127.3±0.2 ppm, 125.5±0.2 ppm, 123.4±0.2 ppm, 119.7±0.2 ppm, 74.3±0.2 ppm, 37.4±0.2 ppm, 36.2±0.2 ppm, 30.6±0.2 ppm, 27.4±0.2 ppm, and 17.7±0.2 ppm, and (b) one, two, three, four, five, six, seven, eight, nine, ten, or more peaks selected from 164.7±0.2 ppm, 163.7±0.2 ppm, 154.4±0.2 ppm, 138.8±0.2 ppm, 131.5±0.2 ppm, 120.7±0.2 ppm, 118.1±0.2 ppm, 75.7±0.2 ppm, 73.4±0.2 ppm, 21.9±0.2 ppm, and 19.4±0.2 ppm.

In some embodiments, crystalline Compound I cyclohexane solvate Form A is characterized as having a ¹³C SSNMR spectrum with twelve or more peaks selected from 166.6±0.2 ppm, 164.7±0.2 ppm, 163.7±0.2 ppm, 154.4±0.2 ppm, 152.1±0.2 ppm, 150.8±0.2 ppm, 140.4±0.2 ppm, 138.8±0.2 ppm, 137.6±0.2 ppm, 135.4±0.2 ppm, 131.5±0.2 ppm, 127.3±0.2 ppm, 125.5±0.2 ppm, 123.4±0.2 ppm, 120.7±0.2 ppm, 119.7±0.2 ppm, 118.1±0.2 ppm, 75.7±0.2 ppm, 74.3±0.2 ppm, 73.4±0.2 ppm, 37.4±0.2 ppm, 36.2±0.2 ppm, 30.6±0.2 ppm, 27.4±0.2 ppm, 21.9±0.2 ppm, 19.4±0.2 ppm, and 17.7±0.2 ppm.

In some embodiments, crystalline Compound I cyclohexane solvate Form A is characterized as having a ¹³C SSNMR spectrum with peaks at 166.6±0.2 ppm, 164.7±0.2 ppm, 163.7±0.2 ppm, 154.4±0.2 ppm, 152.1±0.2 ppm, 150.8±0.2 ppm, 140.4±0.2 ppm, 138.8±0.2 ppm, 137.6±0.2 ppm, 135.4±0.2 ppm, 131.5±0.2 ppm, 127.3±0.2 ppm, 125.5±0.2 ppm, 123.4±0.2 ppm, 120.7±0.2 ppm, 119.7±0.2 ppm, 118.1±0.2 ppm, 75.7±0.2 ppm, 74.3±0.2 ppm, 73.4±0.2 ppm, 37.4±0.2 ppm, 36.2±0.2 ppm, 30.6±0.2 ppm, 27.4±0.2 ppm, 21.9±0.2 ppm, 19.4±0.2 ppm, and 17.7±0.2 ppm.

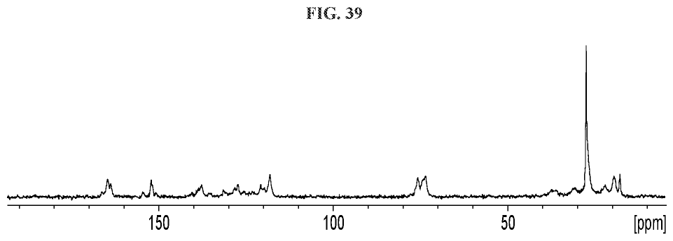

In some embodiments, crystalline Compound I cyclohexane solvate Form A is characterized by a ¹³C SSNMR spectrum substantially similar to FIG. 39.

In some embodiments, crystalline Compound I cyclohexane solvate Form A is characterized as having a ¹⁹F SSNMR spectrum with a peak at −62.6±0.2 ppm. In some embodiments, crystalline Compound I cyclohexane solvate Form A is characterized as having a ¹⁹F SSNMR spectrum with a peak at −65.9±0.2 ppm. In some embodiments, crystalline Compound I cyclohexane solvate Form A is characterized as having a ¹⁹F SSNMR spectrum with a peak at −66.8±0.2 ppm. In some embodiments, crystalline Compound I cyclohexane solvate Form A is characterized as having a ¹⁹F SSNMR spectrum with a peak at −75.4±0.2 ppm. In some embodiments, crystalline Compound I cyclohexane solvate Form A is characterized as having a ¹⁹F SSNMR spectrum with a peak at −77.6±0.2 ppm.

In some embodiments, crystalline Compound I cyclohexane solvate Form A is characterized as having a ¹⁹F SSNMR spectrum with one, two, three, or four peaks selected from −62.6±0.2 ppm, −65.9±0.2 ppm, −66.8±0.2 ppm, −75.4±0.2 ppm, and −77.6±0.2 ppm. In some embodiments, crystalline Compound I cyclohexane solvate Form A is characterized as having a ¹⁹F SSNMR spectrum with peaks at −62.6±0.2 ppm, −65.9±0.2 ppm, −66.8±0.2 ppm, −75.4±0.2 ppm, and −77.6±0.2 ppm.

In some embodiments, crystalline Compound I cyclohexane solvate Form A is characterized as having a ¹⁹F SSNMR spectrum with (a) one, two, three, four, or five peaks selected from −62.6±0.2 ppm, −65.9±0.2 ppm, −66.8±0.2 ppm, −75.4±0.2 ppm, and −77.6±0.2 ppm, and (b) one or two peaks selected from −64.5±0.2 ppm and −76.6±0.2 ppm.

In some embodiments, crystalline Compound I cyclohexane solvate Form A is characterized as having a ¹⁹F SSNMR spectrum with three, four, five, six, or seven peaks selected from −62.6±0.2 ppm, −64.5±0.2 ppm, −65.9±0.2 ppm, −66.8±0.2 ppm, −75.4±0.2 ppm, −76.6±0.2 ppm, and −77.6±0.2 ppm.

In some embodiments, crystalline Compound I cyclohexane solvate Form A is characterized as having a ¹⁹F SSNMR spectrum with peaks at −62.6±0.2 ppm, −64.5±0.2 ppm, −65.9±0.2 ppm, −66.8±0.2 ppm, −75.4±0.2 ppm, −76.6±0.2 ppm, and −77.6±0.2 ppm.

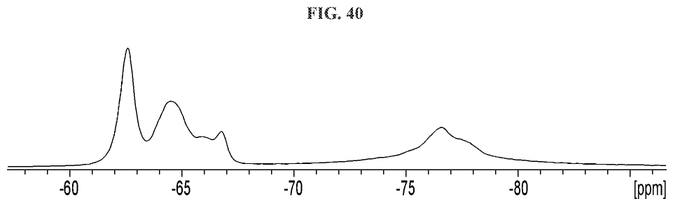

In some embodiments, crystalline Compound I cyclohexane solvate Form A is characterized by a ¹⁹F SSNMR spectrum substantially similar to FIG. 40.

Another aspect of the invention provides a method of making crystalline Compound I cyclohexane solvate Form A. In some embodiments, the method of making crystalline Compound I cyclohexane solvate Form A comprises: (i) adding cyclohexane to crystalline Compound I neat Form D and (ii) shaking the mixture at 25° C. for 3 days to yield crystalline Compound I cyclohexane solvate Form A.

### L. Crystalline Compound I Cyclohexane Solvate Form B

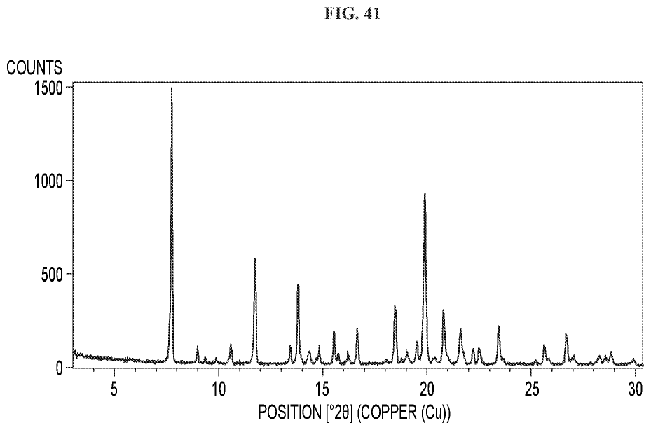

In some embodiments, the invention provides crystalline Compound I cyclohexane solvate Form B. FIG. 41 provides an X-ray powder diffractogram of crystalline Compound I cyclohexane solvate Form B.

In some embodiments, crystalline Compound I cyclohexane solvate Form B is substantially pure. In some embodiments, crystalline Compound I cyclohexane solvate Form B is substantially crystalline. In some embodiments, crystalline Compound I cyclohexane solvate Form B is characterized by an X-ray powder diffractogram generated by an X-ray powder diffraction analysis with an incident beam of Cu Ku radiation.

In some embodiments, crystalline Compound I cyclohexane solvate Form B is characterized by an X-ray powder diffractogram having a signal at 15.5±0.2 degrees two-theta. In some embodiments, crystalline Compound I cyclohexane solvate Form B is characterized by an X-ray powder diffractogram having a signal at 20.8±0.2 degrees two-theta. In some embodiments, crystalline Compound I cyclohexane solvate Form B is characterized by an X-ray powder diffractogram having a signal at 23.4±0.2 degrees two-theta. In some embodiments, crystalline Compound I cyclohexane solvate Form B is characterized by an X-ray powder diffractogram having a signal at 26.7±0.2 degrees two-theta.

In some embodiments, crystalline Compound I cyclohexane solvate Form B is characterized by an X-ray powder diffractogram having a signal at one, two, or three of 15.5±0.2 degrees two-theta, 20.8±0.2 degrees two-theta, 23.4±0.2 degrees two-theta, and 26.7±0.2 degrees two-theta. In some embodiments, crystalline Compound I cyclohexane solvate Form B is characterized by an X-ray powder diffractogram having signals at 15.5±0.2 degrees two-theta, 20.8±0.2 degrees two-theta, 23.4±0.2 degrees two-theta, and 26.7±0.2 degrees two-theta.

In some embodiments, crystalline Compound I cyclohexane solvate Form B is characterized by an X-ray powder diffractogram having signals at (a) one, two, three, or four of 15.5±0.2 degrees two-theta, 20.8±0.2 degrees two-theta, 23.4±0.2 degrees two-theta, and 26.7±0.2 degrees two-theta, and (b) one, two, three, four, five, six, or seven of 7.8±0.2 degrees two-theta, 11.8±0.2 degrees two-theta, 13.8±0.2 degrees two-theta, 16.6±0.2 degrees two-theta, 18.4±0.2 degrees two-theta, 19.9±0.2 degrees two-theta, and 21.6±0.2 degrees two-theta.

In some embodiments, crystalline Compound I cyclohexane solvate Form B is characterized by an X-ray powder diffractogram having signals at five, six, seven, eight, nine, ten, or more of 7.8±0.2 degrees two-theta, 11.8±0.2 degrees two-theta, 13.8±0.2 degrees two-theta, 15.5±0.2 degrees two-theta, 16.6±0.2 degrees two-theta, 18.4±0.2 degrees two-theta, 19.9±0.2 degrees two-theta, 20.8±0.2 degrees two-theta, 21.6±0.2 degrees two-theta, 23.4±0.2 degrees two-theta, and 26.7±0.2 degrees two-theta.

In some embodiments, crystalline Compound I cyclohexane solvate Form B is characterized by an X-ray powder diffractogram having signals at 7.8±0.2 degrees two-theta, 11.8±0.2 degrees two-theta, 13.8±0.2 degrees two-theta, 15.5±0.2 degrees two-theta, 16.6±0.2 degrees two-theta, 18.4±0.2 degrees two-theta, 19.9±0.2 degrees two-theta, 20.8±0.2 degrees two-theta, 21.6±0.2 degrees two-theta, 23.4±0.2 degrees two-theta, and 26.7±0.2 degrees two-theta.

In some embodiments, crystalline Compound I cyclohexane solvate Form B is characterized by an X-ray powder diffractogram substantially similar to FIG. 41.

In some embodiments, crystalline Compound I cyclohexane solvate Form B is characterized as having a ¹³C SSNMR spectrum with a peak at 128.0±0.2 ppm. In some embodiments, crystalline Compound I cyclohexane solvate Form B is characterized as having a ¹³C SSNMR spectrum with a peak at 34.7±0.2 ppm. In some embodiments, crystalline Compound I cyclohexane solvate Form B is characterized as having a ¹³C SSNMR spectrum with a peak at 31.5±0.2 ppm. In some embodiments, crystalline Compound I cyclohexane solvate Form B is characterized as having a ¹³C SSNMR spectrum with a peak at 26.5±0.2 ppm. In some embodiments, crystalline Compound I cyclohexane solvate Form B is characterized as having a ¹³C SSNMR spectrum with a peak at 19.0±0.2 ppm.

In some embodiments, crystalline Compound I cyclohexane solvate Form B is characterized as having a ¹³C SSNMR spectrum with one, two, three, four, or five peaks selected from 128.0±0.2 ppm, 34.7±0.2 ppm, 31.5±0.2 ppm, 26.5±0.2 ppm, and 19.0±0.2 ppm.

In some embodiments, crystalline Compound I cyclohexane solvate Form B is characterized as having a ¹³C SSNMR spectrum with (a) one, two, three, four, or five peaks selected from 128.0±0.2 ppm, 34.7±0.2 ppm, 31.5±0.2 ppm, 26.5±0.2 ppm, and 19.0±0.2 ppm, and (b) one, two, three, four, five, six, seven, eight, nine, ten, or more peaks selected from 164.7±0.2 ppm, 150.9±0.2 ppm, 138.7±0.2 ppm, 118.2±0.2 ppm, 75.6±0.2 ppm, 73.6±0.2 ppm, 36.5±0.2 ppm, and 19.5±0.2 ppm.

In some embodiments, crystalline Compound I cyclohexane solvate Form B is characterized as having a ¹³C SSNMR spectrum with nine, ten, or more peaks selected from 164.7±0.2 ppm, 150.9±0.2 ppm, 138.7±0.2 ppm, 128.0±0.2 ppm, 118.2±0.2 ppm, 75.6±0.2 ppm, 73.6±0.2 ppm, 36.5±0.2 ppm, 34.7±0.2 ppm, 31.5±0.2 ppm, 26.5±0.2 ppm, 19.5±0.2 ppm, and 19.0±0.2 ppm.

In some embodiments, crystalline Compound I cyclohexane solvate Form B is characterized as having a ¹³C SSNMR spectrum with peaks at 164.7±0.2 ppm, 150.9±0.2 ppm, 138.7±0.2 ppm, 128.0±0.2 ppm, 118.2±0.2 ppm, 75.6±0.2 ppm, 73.6±0.2 ppm, 36.5±0.2 ppm, 34.7±0.2 ppm, 31.5±0.2 ppm, 26.5±0.2 ppm, 19.5±0.2 ppm, and 19.0±0.2 ppm.

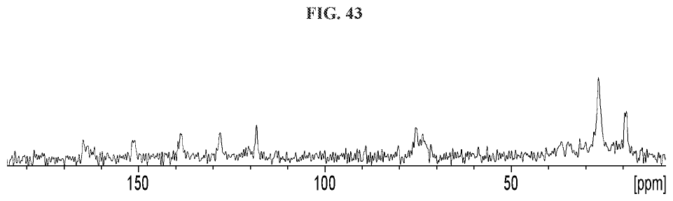

In some embodiments, crystalline Compound I cyclohexane solvate Form B is characterized by a ¹³C SSNMR spectrum substantially similar to FIG. 43.

In some embodiments, crystalline Compound I cyclohexane solvate Form B is characterized as having a ¹⁹F SSNMR spectrum with a peak at −75.0±0.2 ppm.

In some embodiments, crystalline Compound I cyclohexane solvate Form B is characterized as having a ¹⁹F SSNMR spectrum with peaks at −64.3±0.2 ppm and −75.0±0.2 ppm.

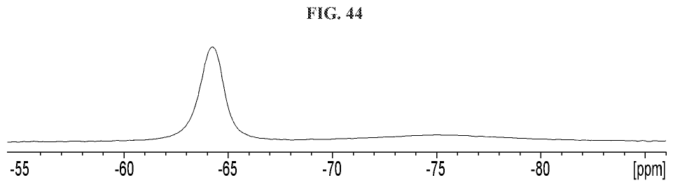

In some embodiments, crystalline Compound I cyclohexane solvate Form B is characterized by a ¹⁹F SSNMR spectrum substantially similar to FIG. 44.

Another aspect of the invention provides a method of making crystalline Compound I cyclohexane solvate Form B. In some embodiments, the method of making crystalline Compound I cyclohexane solvate Form B comprises: (i) adding cyclohexane to crystalline Compound I hemihydrate Form C and (ii) shaking the mixture at 80° C. for 3 days to yield crystalline Compound I cyclohexane solvate Form B.

### M. Crystalline Compound I Cyclohexane Solvate Form C

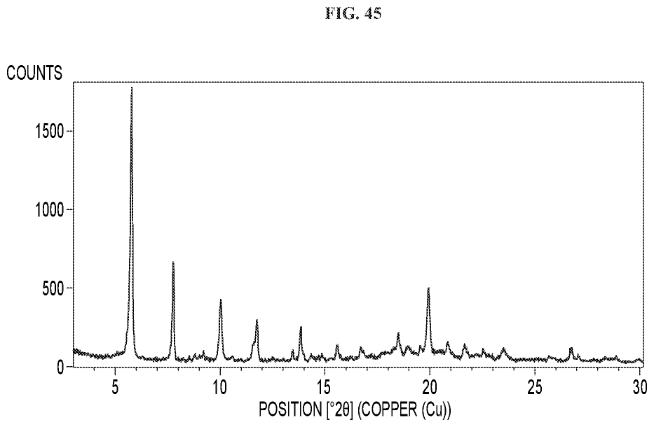

In some embodiments, the invention provides crystalline Compound I cyclohexane solvate Form C. FIG. 45 provides an X-ray powder diffractogram of crystalline Compound I cyclohexane solvate Form C.

In some embodiments, crystalline Compound I cyclohexane solvate Form C is substantially pure. In some embodiments, crystalline Compound I cyclohexane solvate Form C is substantially crystalline. In some embodiments, crystalline Compound I cyclohexane solvate Form C is characterized by an X-ray powder diffractogram generated by an X-ray powder diffraction analysis with an incident beam of Cu Ku radiation.

In some embodiments, crystalline Compound I cyclohexane solvate Form C is characterized by an X-ray powder diffractogram having a signal at 10.0±0.2 degrees two-theta.

In some embodiments, crystalline Compound I cyclohexane solvate Form C is characterized by an X-ray powder diffractogram having (a) a signal at 10.0±0.2 degrees two-theta, and (b) a signal at one, two, three, four, or five of 5.8±0.2 degrees two-theta, 7.8±0.2 degrees two-theta, 11.8±0.2 degrees two-theta, 13.9±0.2 degrees two-theta, and 19.9±0.2 degrees two-theta.

In some embodiments, crystalline Compound I cyclohexane solvate Form C is characterized by an X-ray powder diffractogram having signals at 5.8±0.2 degrees two-theta, 7.8±0.2 degrees two-theta, 10.0±0.2 degrees two-theta, 11.8±0.2 degrees two-theta, 13.9±0.2 degrees two-theta, and 19.9±0.2 degrees two-theta.

In some embodiments, crystalline Compound I cyclohexane solvate Form C is characterized by an X-ray powder diffractogram substantially similar to FIG. 45.

Another aspect of the invention provides a method of making crystalline Compound I cyclohexane solvate Form C. In some embodiments, the method of making crystalline Compound I cyclohexane solvate Form C comprises: (i) adding cyclohexane to crystalline Compound I hemihydrate Form C and (ii) shaking the mixture at 60° C. for one week to yield crystalline Compound I cyclohexane solvate Form C.

### N. Crystalline Compound I Ethanol Solvate

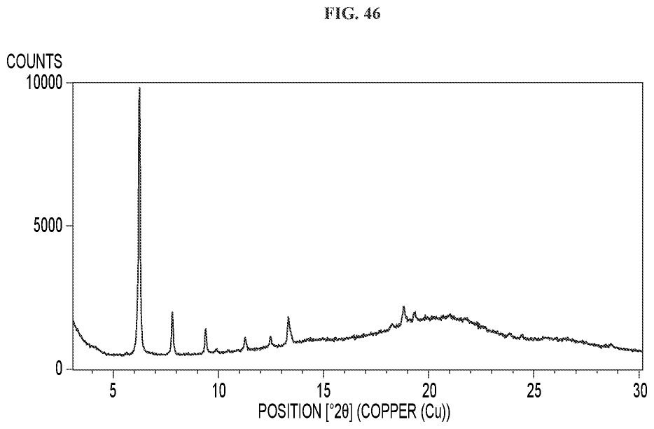

In some embodiments, the invention provides crystalline Compound I ethanol solvate. FIG. 46 provides an X-ray powder diffractogram of crystalline Compound I ethanol solvate.

In some embodiments, crystalline Compound I ethanol solvate is substantially pure. In some embodiments, crystalline Compound I ethanol solvate is substantially crystalline. In some embodiments, crystalline Compound I ethanol solvate is characterized by an X-ray powder diffractogram generated by an X-ray powder diffraction analysis with an incident beam of Cu Kα radiation.

In some embodiments, crystalline Compound I ethanol solvate is characterized by an X-ray powder diffractogram having a signal at 6.2±0.2 degrees two-theta. In some embodiments, crystalline Compound I ethanol solvate is characterized by an X-ray powder diffractogram having a signal at 7.8±0.2 degrees two-theta. In some embodiments, crystalline Compound I ethanol solvate is characterized by an X-ray powder diffractogram having a signal at 13.3±0.2 degrees two-theta.

In some embodiments, crystalline Compound I ethanol solvate is characterized by an X-ray powder diffractogram having a signal at one, two, or three of 6.2±0.2 degrees two-theta, 7.8±0.2 degrees two-theta, and 13.3±0.2 degrees two-theta.

In some embodiments, crystalline Compound I ethanol solvate is characterized by an X-ray powder diffractogram substantially similar to FIG. 46.

In some embodiments, crystalline Compound I ethanol solvate is characterized as having a ¹³C SSNMR spectrum with a peak at 162.8±0.2 ppm. In some embodiments, crystalline Compound I ethanol solvate is characterized as having a ¹³C SSNMR spectrum with a peak at 151.7±0.2 ppm. In some embodiments, crystalline Compound I ethanol solvate is characterized as having a ¹³C SSNMR spectrum with a peak at 150.7±0.2 ppm. In some embodiments, crystalline Compound I ethanol solvate is characterized as having a ¹³C SSNMR spectrum with a peak at 139.1±0.2 ppm. In some embodiments, crystalline Compound I ethanol solvate is characterized as having a ¹³C SSNMR spectrum with a peak at 138.0±0.2 ppm. In some embodiments, crystalline Compound I ethanol solvate is characterized as having a ¹³C SSNMR spectrum with a peak at 127.4±0.2 ppm. In some embodiments, crystalline Compound I ethanol solvate is characterized as having a ¹³C SSNMR spectrum with a peak at 126.9±0.2 ppm. In some embodiments, crystalline Compound I ethanol solvate is characterized as having a ¹³C SSNMR spectrum with a peak at 124.3±0.2 ppm. In some embodiments, crystalline Compound I ethanol solvate is characterized as having a ¹³C SSNMR spectrum with a peak at 120.4±0.2 ppm. In some embodiments, crystalline Compound I ethanol solvate is characterized as having a ¹³C SSNMR spectrum with a peak at 117.7±0.2 ppm. In some embodiments, crystalline Compound I ethanol solvate is characterized as having a ¹³C SSNMR spectrum with a peak at 78.7±0.2 ppm. In some embodiments, crystalline Compound I ethanol solvate is characterized as having a ¹³C SSNMR spectrum with a peak at 77.9±0.2 ppm. In some embodiments, crystalline Compound I ethanol solvate is characterized as having a ¹³C SSNMR spectrum with a peak at 72.6±0.2 ppm. In some embodiments, crystalline Compound I ethanol solvate is characterized as having a ¹³C SSNMR spectrum with a peak at 33.4±0.2 ppm. In some embodiments, crystalline Compound I ethanol solvate is characterized as having a ¹³C SSNMR spectrum with a peak at 25.9±0.2 ppm. In some embodiments, crystalline Compound I ethanol solvate is characterized as having a ¹³C SSNMR spectrum with a peak at 21.7±0.2 ppm. In some embodiments, crystalline Compound I ethanol solvate is characterized as having a ¹³C SSNMR spectrum with a peak at 20.0±0.2 ppm. In some embodiments, crystalline Compound I ethanol solvate is characterized as having a ¹³C SSNMR spectrum with a peak at 18.8±0.2 ppm. In some embodiments, crystalline Compound I ethanol solvate is characterized as having a ¹³C SSNMR spectrum with a peak at 17.9±0.2 ppm.

In some embodiments, crystalline Compound I ethanol solvate is characterized as having a ¹³C SSNMR spectrum with one, two, three, four, five, six, seven, eight, nine, ten, or more peaks selected from 162.8±0.2 ppm, 151.7±0.2 ppm, 150.7±0.2 ppm, 139.1±0.2 ppm, 138.0±0.2 ppm, 127.4±0.2 ppm, 126.9±0.2 ppm, 124.3±0.2 ppm, 120.4±0.2 ppm, 117.7±0.2 ppm, 78.7±0.2 ppm, 77.9±0.2 ppm, 72.6±0.2 ppm, 33.4±0.2 ppm, 25.9±0.2 ppm, 21.7±0.2 ppm, 20.0±0.2 ppm, 18.8±0.2 ppm, and 17.9±0.2 ppm.

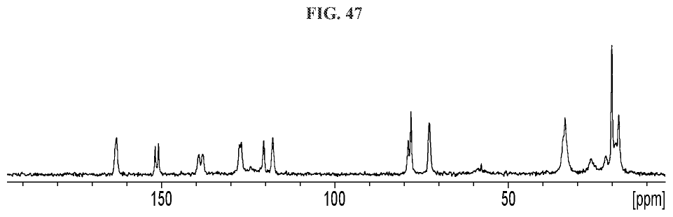

In some embodiments, crystalline Compound I ethanol solvate is characterized by a ¹³C SSNMR spectrum substantially similar to FIG. 47.

In some embodiments, crystalline Compound I ethanol solvate is characterized as having a ¹⁹F SSNMR spectrum with a peak at −63.1±0.2 ppm. In some embodiments, crystalline Compound I ethanol solvate is characterized as having a ¹⁹F SSNMR spectrum with a peak at −64.2±0.2 ppm. In some embodiments, crystalline Compound I ethanol solvate is characterized as having a ¹⁹F SSNMR spectrum with a peak at −78.0±0.2 ppm.

In some embodiments, crystalline Compound I ethanol solvate is characterized as having a ¹⁹F SSNMR spectrum with one, two, or three peaks selected from −63.1±0.2 ppm, −64.2±0.2 ppm, and −78.0±0.2 ppm.

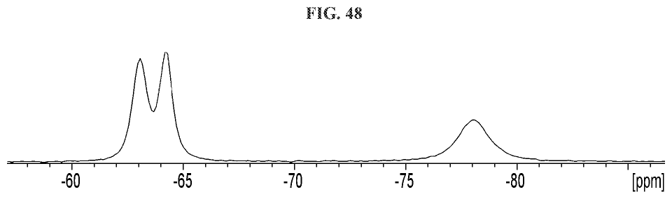

In some embodiments, crystalline Compound I ethanol solvate is characterized by a ¹⁹F SSNMR spectrum substantially similar to FIG. 48.

Another aspect of the invention provides a method of making crystalline Compound I ethanol solvate. In some embodiments, the method of making crystalline Compound I ethanol solvate comprises stirring crystalline Compound I hemihydrate Form C in ethanol at −20° C. to yield crystalline Compound I ethanol solvate.

### O. Crystalline Compound I Solvate/Hydrate (Dry)

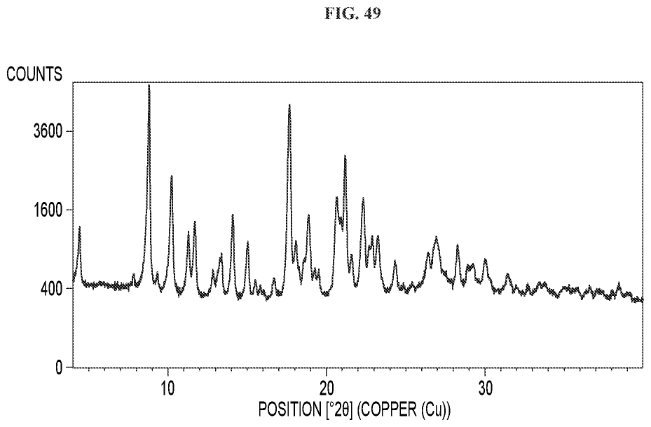

In some embodiments, the invention provides crystalline Compound I solvate/hydrate (dry). FIG. 49 provides an X-ray powder diffractogram of crystalline Compound I solvate/hydrate (dry).

In some embodiments, crystalline Compound I solvate/hydrate (dry) is substantially pure. In some embodiments, crystalline Compound I solvate/hydrate (dry) is substantially crystalline. In some embodiments, crystalline Compound I solvate/hydrate (dry) is characterized by an X-ray powder diffractogram generated by an X-ray powder diffraction analysis with an incident beam of Cu Kα radiation.

In some embodiments, crystalline Compound I solvate/hydrate (dry) is characterized by an X-ray powder diffractogram having (a) a signal at 22.7±0.2 degrees two-theta, and (b) a signal at one, two, three, four, five, six, seven, eight, nine, ten, or more of 4.4±0.2 degrees two-theta, 8.8±0.2 degrees two-theta, 10.3±0.2 degrees two-theta, 11.3±0.2 degrees two-theta, 11.7±0.2 degrees two-theta, 13.4±0.2 degrees two-theta, 14.1±0.2 degrees two-theta, 15.1±0.2 degrees two-theta, 17.7±0.2 degrees two-theta, 18.1±0.2 degrees two-theta, 18.9±0.2 degrees two-theta, 20.6±0.2 degrees two-theta, 21.2±0.2 degrees two-theta, 22.3±0.2 degrees two-theta, 22.9±0.2 degrees two-theta, 23.3±0.2 degrees two-theta, 27.0±0.2 degrees two-theta, and 28.3±0.2 degrees two-theta.

In some embodiments, crystalline Compound I solvate/hydrate (dry) is characterized by an X-ray powder diffractogram having signals at 4.4±0.2 degrees two-theta, 8.8±0.2 degrees two-theta, 10.3±0.2 degrees two-theta, 11.3±0.2 degrees two-theta, 11.7±0.2 degrees two-theta, 13.4±0.2 degrees two-theta, 14.1±0.2 degrees two-theta, 15.1±0.2 degrees two-theta, 17.7±0.2 degrees two-theta, 18.1±0.2 degrees two-theta, 18.9±0.2 degrees two-theta, 20.6±0.2 degrees two-theta, 21.2±0.2 degrees two-theta, 22.3±0.2 degrees two-theta, 22.7±0.2 degrees two-theta, 22.9±0.2 degrees two-theta, 23.3±0.2 degrees two-theta, 27.0±0.2 degrees two-theta, and 28.3±0.2 degrees two-theta.

In some embodiments, crystalline Compound I solvate/hydrate (dry) is characterized by an X-ray powder diffractogram substantially similar to FIG. 49.

Another aspect of the invention provides a method of making crystalline Compound I solvate/hydrate (dry). In some embodiments, the method of making crystalline Compound I solvate/hydrate (dry) comprises: (i) stirring crystalline Compound I heptane solvate Form A in water at room temperature for 2 weeks, (ii) filtering the solids, and (iii) air drying the solids to yield crystalline Compound I solvate/hydrate (dry). In some embodiments, the method of making crystalline Compound I solvate/hydrate (dry) comprises: (i) dissolving crystalline Compound I heptane solvate Form A in ethanol, (i) adding water (water/ethanol=1.23˜3.15), (iii) stirring at 60° C. for 3 days, (iv) filtering the solids, and (v) air drying the solids to yield crystalline Compound I solvate/hydrate (dry).

### P. Crystalline Compound I Solvate/Hydrate (Wet)

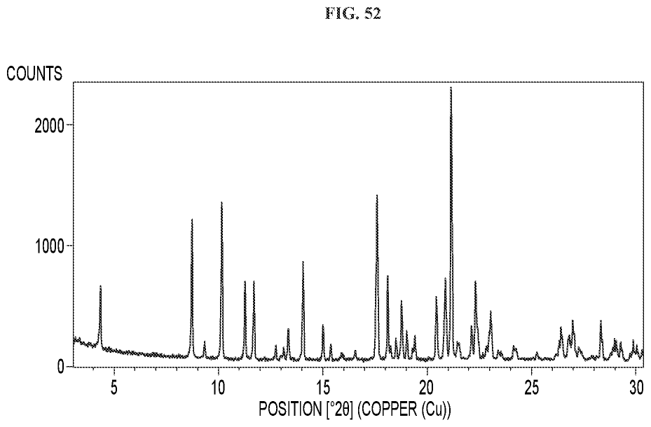

In some embodiments, the invention provides crystalline Compound I solvate/hydrate (wet). FIG. 52 provides an X-ray powder diffractogram of crystalline Compound I solvate/hydrate (wet).

In some embodiments, crystalline Compound I solvate/hydrate (wet) is substantially pure. In some embodiments, crystalline Compound I solvate/hydrate (wet) is substantially crystalline. In some embodiments, crystalline Compound I solvate/hydrate (wet) is characterized by an X-ray powder diffractogram generated by an X-ray powder diffraction analysis with an incident beam of Cu Kα radiation.

In some embodiments, crystalline Compound I solvate/hydrate (wet) is characterized by an X-ray powder diffractogram having (a) a signal at 26.4±0.2 degrees two-theta, and (b) a signal at one or more of 4.4±0.2 degrees two-theta, 8.7±0.2 degrees two-theta, 10.2±0.2 degrees two-theta, 11.3±0.2 degrees two-theta, 11.7±0.2 degrees two-theta, 13.4±0.2 degrees two-theta, 14.1±0.2 degrees two-theta, 15.0±0.2 degrees two-theta, 17.6±0.2 degrees two-theta, 18.1±0.2 degrees two-theta, 18.8±0.2 degrees two-theta, 19.0±0.2 degrees two-theta, 20.4±0.2 degrees two-theta, 20.9±0.2 degrees two-theta, 21.1±0.2 degrees two-theta, 22.1±0.2 degrees two-theta, 22.3±0.2 degrees two-theta, 23.0±0.2 degrees two-theta, 27.0±0.2 degrees two-theta, and 28.3±0.2 degrees two-theta.

In some embodiments, crystalline Compound I solvate/hydrate (wet) is characterized by an X-ray powder diffractogram having signals at 4.4±0.2 degrees two-theta, 8.7±0.2 degrees two-theta, 10.2±0.2 degrees two-theta, 11.3±0.2 degrees two-theta, 11.7±0.2 degrees two-theta, 13.4±0.2 degrees two-theta, 14.1±0.2 degrees two-theta, 15.0±0.2 degrees two-theta, 17.6±0.2 degrees two-theta, 18.1±0.2 degrees two-theta, 18.8±0.2 degrees two-theta, 19.0±0.2 degrees two-theta, 20.4±0.2 degrees two-theta, 20.9±0.2 degrees two-theta, 21.1±0.2 degrees two-theta, 22.1±0.2 degrees two-theta, 22.3±0.2 degrees two-theta, 23.0±0.2 degrees two-theta, 26.4±0.2 degrees two-theta, 27.0±0.2 degrees two-theta, and 28.3±0.2 degrees two-theta.

In some embodiments, crystalline Compound I solvate/hydrate (wet) is characterized by an X-ray powder diffractogram substantially similar to FIG. 52.

In some embodiments, crystalline Compound I solvate/hydrate (wet) is characterized as having a ¹³C SSNMR spectrum with a peak at 163.5±0.2 ppm. In some embodiments, crystalline Compound I solvate/hydrate (wet) is characterized as having a ¹³C SSNMR spectrum with a peak at 162.4±0.2 ppm. In some embodiments, crystalline Compound I solvate/hydrate (wet) is characterized as having a ¹³C SSNMR spectrum with a peak at 151.7±0.2 ppm. In some embodiments, crystalline Compound I solvate/hydrate (wet) is characterized as having a ¹³C SSNMR spectrum with a peak at 139.2±0.2 ppm. In some embodiments, crystalline Compound I solvate/hydrate (wet) is characterized as having a ¹³C SSNMR spectrum with a peak at 137.8±0.2 ppm. In some embodiments, crystalline Compound I solvate/hydrate (wet) is characterized as having a ¹³C SSNMR spectrum with a peak at 128.3±0.2 ppm. In some embodiments, crystalline Compound I solvate/hydrate (wet) is characterized as having a ¹³C SSNMR spectrum with a peak at 126.4±0.2 ppm. In some embodiments, crystalline Compound I solvate/hydrate (wet) is characterized as having a ¹³C SSNMR spectrum with a peak at 124.4±0.2 ppm. In some embodiments, crystalline Compound I solvate/hydrate (wet) is characterized as having a ¹³C SSNMR spectrum with a peak at 122.2±0.2 ppm. In some embodiments, crystalline Compound I solvate/hydrate (wet) is characterized as having a ¹³C SSNMR spectrum with a peak at 118.4±0.2 ppm. In some embodiments, crystalline Compound I solvate/hydrate (wet) is characterized as having a ¹³C SSNMR spectrum with a peak at 116.8±0.2 ppm. In some embodiments, crystalline Compound I solvate/hydrate (wet) is characterized as having a ¹³C SSNMR spectrum with a peak at 77.8±0.2 ppm. In some embodiments, crystalline Compound I solvate/hydrate (wet) is characterized as having a ¹³C SSNMR spectrum with a peak at 77.6±0.2 ppm. In some embodiments, crystalline Compound I solvate/hydrate (wet) is characterized as having a ¹³C SSNMR spectrum with a peak at 72.9±0.2 ppm. In some embodiments, crystalline Compound I solvate/hydrate (wet) is characterized as having a ¹³C SSNMR spectrum with a peak at 72.5±0.2 ppm. In some embodiments, crystalline Compound I solvate/hydrate (wet) is characterized as having a ¹³C SSNMR spectrum with a peak at 36.9±0.2 ppm. In some embodiments, crystalline Compound I solvate/hydrate (wet) is characterized as having a ¹³C SSNMR spectrum with a peak at 35.6±0.2 ppm. In some embodiments, crystalline Compound I solvate/hydrate (wet) is characterized as having a ¹³C SSNMR spectrum with a peak at 33.9±0.2 ppm. In some embodiments, crystalline Compound I solvate/hydrate (wet) is characterized as having a ¹³C SSNMR spectrum with a peak at 25.6±0.2 ppm. In some embodiments, crystalline Compound I solvate/hydrate (wet) is characterized as having a ¹³C SSNMR spectrum with a peak at 25.2±0.2 ppm. In some embodiments, crystalline Compound I solvate/hydrate (wet) is characterized as having a ¹³C SSNMR spectrum with a peak at 22.5±0.2 ppm. In some embodiments, crystalline Compound I solvate/hydrate (wet) is characterized as having a ¹³C SSNMR spectrum with a peak at 21.0±0.2 ppm. In some embodiments, crystalline Compound I solvate/hydrate (wet) is characterized as having a ¹³C SSNMR spectrum with a peak at 20.0±0.2 ppm. In some embodiments, crystalline Compound I solvate/hydrate (wet) is characterized as having a ¹³C SSNMR spectrum with a peak at 17.2±0.2 ppm.

In some embodiments, crystalline Compound I solvate/hydrate (wet) is characterized as having a ¹³C SSNMR spectrum with one, two, three, four, five, six, seven, eight, nine, ten, or more peaks selected from 163.5±0.2 ppm, 162.4±0.2 ppm, 151.7±0.2 ppm, 139.2±0.2 ppm, 137.8±0.2 ppm, 128.3±0.2 ppm, 126.4±0.2 ppm, 124.4±0.2 ppm, 122.2±0.2 ppm, 118.4±0.2 ppm, 116.8±0.2 ppm, 77.8±0.2 ppm, 77.6±0.2 ppm, 72.9±0.2 ppm, 72.5±0.2 ppm, 36.9±0.2 ppm, 35.6±0.2 ppm, 33.9±0.2 ppm, 25.6±0.2 ppm, 25.2±0.2 ppm, 22.5±0.2 ppm, 21.0±0.2 ppm, 20.0±0.2 ppm, and 17.2±0.2 ppm.

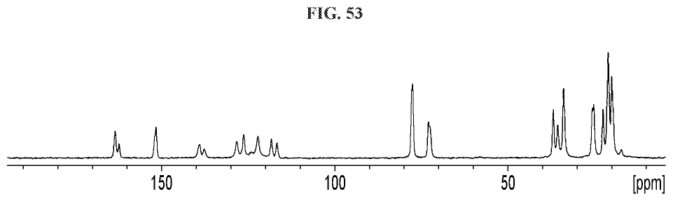

In some embodiments, crystalline Compound I solvate/hydrate (wet) is characterized by a ¹³C SSNMR spectrum substantially similar to FIG. 53.

In some embodiments, crystalline Compound I solvate/hydrate (wet) is characterized as having a ¹⁹F SSNMR spectrum with a peak at −62.3±0.2 ppm. In some embodiments, crystalline Compound I solvate/hydrate (wet) is characterized as having a ¹⁹F SSNMR spectrum with a peak at −64.5±0.2 ppm. In some embodiments, crystalline Compound I solvate/hydrate (wet) is characterized as having a ¹⁹F SSNMR spectrum with a peak at −76.1±0.2 ppm. In some embodiments, crystalline Compound I solvate/hydrate (wet) is characterized as having a ¹⁹F SSNMR spectrum with a peak at −78.2±0.2 ppm.

In some embodiments, crystalline Compound I solvate/hydrate (wet) is characterized as having a ¹⁹F SSNMR spectrum with one, two, three, or four peaks at −62.3±0.2 ppm, −64.5±0.2 ppm, −76.1±0.2 ppm, and −78.2±0.2 ppm.

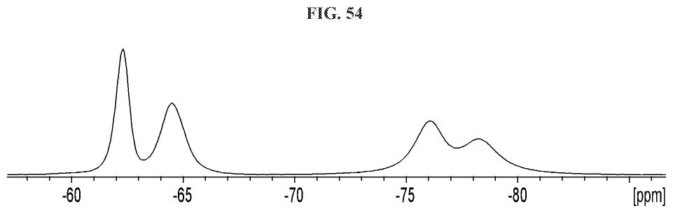

In some embodiments, crystalline Compound I solvate/hydrate (wet) is characterized by a ¹⁹F SSNMR spectrum substantially similar to FIG. 54.

Another aspect of the invention provides a method of making crystalline Compound I solvate/hydrate (wet). In some embodiments, the method of making crystalline Compound I solvate/hydrate (wet) comprises: (i) adding ethanol/water 50:50 (% V/V) to crystalline Compound I hemihydrate Form C and (ii) stirring at 5° C. to yield crystalline Compound I solvate/hydrate (wet).

### Q. Crystalline Compound I L-Lysine Cocrystal

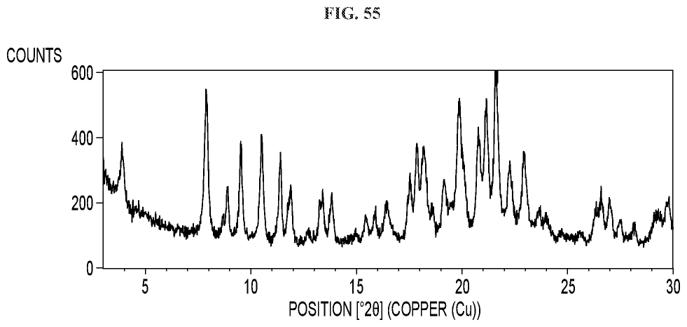

In some embodiments, the invention provides crystalline Compound I L-lysine cocrystal. FIG. 55 provides an X-ray powder diffractogram of crystalline Compound I L-lysine cocrystal.

In some embodiments, crystalline Compound I L-lysine cocrystal is substantially pure. In some embodiments, crystalline Compound I L-lysine cocrystal is substantially crystalline. In some embodiments, crystalline Compound I L-lysine cocrystal is characterized by an X-ray powder diffractogram generated by an X-ray powder diffraction analysis with an incident beam of Cu Kα radiation.

In some embodiments, crystalline Compound I L-lysine cocrystal is characterized by an X-ray powder diffractogram having a signal at 3.9±0.2 degrees two-theta. In some embodiments, crystalline Compound I L-lysine cocrystal is characterized by an X-ray powder diffractogram having a signal at 7.9±0.2 degrees two-theta. In some embodiments, crystalline Compound I L-lysine cocrystal is characterized by an X-ray powder diffractogram having a signal at 8.9±0.2 degrees two-theta. In some embodiments, crystalline Compound I L-lysine cocrystal is characterized by an X-ray powder diffractogram having a signal at 9.5±0.2 degrees two-theta. In some embodiments, crystalline Compound I L-lysine cocrystal is characterized by an X-ray powder diffractogram having a signal at 10.5±0.2 degrees two-theta. In some embodiments, crystalline Compound I L-lysine cocrystal is characterized by an X-ray powder diffractogram having a signal at 11.4±0.2 degrees two-theta. In some embodiments, crystalline Compound I L-lysine cocrystal is characterized by an X-ray powder diffractogram having a signal at 11.8±0.2 degrees two-theta. In some embodiments, crystalline Compound I L-lysine cocrystal is characterized by an X-ray powder diffractogram having a signal at 13.3±0.2 degrees two-theta. In some embodiments, crystalline Compound I L-lysine cocrystal is characterized by an X-ray powder diffractogram having a signal at 13.4±0.2 degrees two-theta. In some embodiments, crystalline Compound I L-lysine cocrystal is characterized by an X-ray powder diffractogram having a signal at 13.8±0.2 degrees two-theta. In some embodiments, crystalline Compound I L-lysine cocrystal is characterized by an X-ray powder diffractogram having a signal at 15.4±0.2 degrees two-theta. In some embodiments, crystalline Compound I L-lysine cocrystal is characterized by an X-ray powder diffractogram having a signal at 15.9±0.2 degrees two-theta. In some embodiments, crystalline Compound I L-lysine cocrystal is characterized by an X-ray powder diffractogram having a signal at 16.4±0.2 degrees two-theta. In some embodiments, crystalline Compound I L-lysine cocrystal is characterized by an X-ray powder diffractogram having a signal at 17.5±0.2 degrees two-theta. In some embodiments, crystalline Compound I L-lysine cocrystal is characterized by an X-ray powder diffractogram having a signal at 17.8±0.2 degrees two-theta. In some embodiments, crystalline Compound I L-lysine cocrystal is characterized by an X-ray powder diffractogram having a signal at 18.2±0.2 degrees two-theta. In some embodiments, crystalline Compound I L-lysine cocrystal is characterized by an X-ray powder diffractogram having a signal at 18.6±0.2 degrees two-theta. In some embodiments, crystalline Compound I L-lysine cocrystal is characterized by an X-ray powder diffractogram having a signal at 19.2±0.2 degrees two-theta. In some embodiments, crystalline Compound I L-lysine cocrystal is characterized by an X-ray powder diffractogram having a signal at 19.9±0.2 degrees two-theta. In some embodiments, crystalline Compound I L-lysine cocrystal is characterized by an X-ray powder diffractogram having a signal at 20.8±0.2 degrees two-theta. In some embodiments, crystalline Compound I L-lysine cocrystal is characterized by an X-ray powder diffractogram having a signal at 21.1±0.2 degrees two-theta. In some embodiments, crystalline Compound I L-lysine cocrystal is characterized by an X-ray powder diffractogram having a signal at 21.6±0.2 degrees two-theta. In some embodiments, crystalline Compound I L-lysine cocrystal is characterized by an X-ray powder diffractogram having a signal at 22.3±0.2 degrees two-theta. In some embodiments, crystalline Compound I L-lysine cocrystal is characterized by an X-ray powder diffractogram having a signal at 22.9±0.2 degrees two-theta. In some embodiments, crystalline Compound I L-lysine cocrystal is characterized by an X-ray powder diffractogram having a signal at 23.6±0.2 degrees two-theta. In some embodiments, crystalline Compound I L-lysine cocrystal is characterized by an X-ray powder diffractogram having a signal at 26.3±0.2 degrees two-theta. In some embodiments, crystalline Compound I L-lysine cocrystal is characterized by an X-ray powder diffractogram having a signal at 26.6±0.2 degrees two-theta. In some embodiments, crystalline Compound I L-lysine cocrystal is characterized by an X-ray powder diffractogram having a signal at 27.0±0.2 degrees two-theta. In some embodiments, crystalline Compound I L-lysine cocrystal is characterized by an X-ray powder diffractogram having a signal at 27.5±0.2 degrees two-theta. In some embodiments, crystalline Compound I L-lysine cocrystal is characterized by an X-ray powder diffractogram having a signal at 29.2±0.2 degrees two-theta. In some embodiments, crystalline Compound I L-lysine cocrystal is characterized by an X-ray powder diffractogram having a signal at 29.7±0.2 degrees two-theta.

In some embodiments, crystalline Compound I L-lysine cocrystal is characterized by an X-ray powder diffractogram having a signal at one, two, three, four, or five of 7.9±0.2 degrees two-theta, 10.5±0.2 degrees two-theta, 19.9±0.2 degrees two-theta, 21.1±0.2 degrees two-theta, and 21.6±0.2 degrees two-theta.

In some embodiments, crystalline Compound I L-lysine cocrystal is characterized by an X-ray powder diffractogram having a signal at one, two, three, four, five, six, seven, eight, nine, or ten of 7.9±0.2 degrees two-theta, 9.5±0.2 degrees two-theta, 10.5±0.2 degrees two-theta, 11.4±0.2 degrees two-theta, 17.8±0.2 degrees two-theta, 19.9±0.2 degrees two-theta, 20.8±0.2 degrees two-theta, 21.1±0.2 degrees two-theta, 21.6±0.2 degrees two-theta, and 22.9±0.2 degrees two-theta.

In some embodiments, crystalline Compound I L-lysine cocrystal is characterized by an X-ray powder diffractogram having signals at one, two, three, four, five, six, seven, eight, nine, ten, or more of 3.9±0.2 degrees two-theta, 7.9±0.2 degrees two-theta, 8.9±0.2 degrees two-theta, 9.5±0.2 degrees two-theta, 10.5±0.2 degrees two-theta, 11.4±0.2 degrees two-theta, 11.8±0.2 degrees two-theta, 13.3±0.2 degrees two-theta, 13.4±0.2 degrees two-theta, 13.8±0.2 degrees two-theta, 15.4±0.2 degrees two-theta, 15.9±0.2 degrees two-theta, 16.4±0.2 degrees two-theta, 17.5±0.2 degrees two-theta, 17.8±0.2 degrees two-theta, 18.2±0.2 degrees two-theta, 18.6±0.2 degrees two-theta, 19.2±0.2 degrees two-theta, 19.9±0.2 degrees two-theta, 20.8±0.2 degrees two-theta, 21.1±0.2 degrees two-theta, 21.6±0.2 degrees two-theta, 22.3±0.2 degrees two-theta, 22.9±0.2 degrees two-theta, 23.6±0.2 degrees two-theta, 26.3±0.2 degrees two-theta, 26.6±0.2 degrees two-theta, 27.0±0.2 degrees two-theta, 27.5±0.2 degrees two-theta, 29.2±0.2 degrees two-theta, and 29.7±0.2 degrees two-theta. In some embodiments, crystalline Compound I L-lysine cocrystal is characterized by an X-ray powder diffractogram having signals at 3.9±0.2 degrees two-theta, 7.9±0.2 degrees two-theta, 8.9±0.2 degrees two-theta, 9.5±0.2 degrees two-theta, 10.5±0.2 degrees two-theta, 11.4±0.2 degrees two-theta, 11.8±0.2 degrees two-theta, 13.3±0.2 degrees two-theta, 13.4±0.2 degrees two-theta, 13.8±0.2 degrees two-theta, 15.4±0.2 degrees two-theta, 15.9±0.2 degrees two-theta, 16.4±0.2 degrees two-theta, 17.5±0.2 degrees two-theta, 17.8±0.2 degrees two-theta, 18.2±0.2 degrees two-theta, 18.6±0.2 degrees two-theta, 19.2±0.2 degrees two-theta, 19.9±0.2 degrees two-theta, 20.8±0.2 degrees two-theta, 21.1±0.2 degrees two-theta, 21.6±0.2 degrees two-theta, 22.3±0.2 degrees two-theta, 22.9±0.2 degrees two-theta, 23.6±0.2 degrees two-theta, 26.3±0.2 degrees two-theta, 26.6±0.2 degrees two-theta, 27.0±0.2 degrees two-theta, 27.5±0.2 degrees two-theta, 29.2±0.2 degrees two-theta, and 29.7±0.2 degrees two-theta.

In some embodiments, crystalline Compound I L-lysine cocrystal is characterized by an X-ray powder diffractogram substantially similar to FIG. 55.

In some embodiments, crystalline Compound I L-lysine cocrystal is characterized as having a ¹³C SSNMR spectrum with a peak at 181.6±0.2 ppm. In some embodiments, crystalline Compound I L-lysine cocrystal is characterized as having a ¹³C SSNMR spectrum with a peak at 180.9±0.2 ppm. In some embodiments, crystalline Compound I L-lysine cocrystal is characterized as having a ¹³C SSNMR spectrum with a peak at 177.5±0.2 ppm. In some embodiments, crystalline Compound I L-lysine cocrystal is characterized as having a ¹³C SSNMR spectrum with a peak at 165.4±0.2 ppm. In some embodiments, crystalline Compound I L-lysine cocrystal is characterized as having a ¹³C SSNMR spectrum with a peak at 164.4±0.2 ppm. In some embodiments, crystalline Compound I L-lysine cocrystal is characterized as having a ¹³C SSNMR spectrum with a peak at 163.7±0.2 ppm. In some embodiments, crystalline Compound I L-lysine cocrystal is characterized as having a ¹³C SSNMR spectrum with a peak at 162.7±0.2 ppm. In some embodiments, crystalline Compound I L-lysine cocrystal is characterized as having a ¹³C SSNMR spectrum with a peak at 151.9±0.2 ppm. In some embodiments, crystalline Compound I L-lysine cocrystal is characterized as having a ¹³C SSNMR spectrum with a peak at 150.7±0.2 ppm. In some embodiments, crystalline Compound I L-lysine cocrystal is characterized as having a ¹³C SSNMR spectrum with a peak at 138.9±0.2 ppm. In some embodiments, crystalline Compound I L-lysine cocrystal is characterized as having a ¹³C SSNMR spectrum with a peak at 138.2±0.2 ppm. In some embodiments, crystalline Compound I L-lysine cocrystal is characterized as having a ¹³C SSNMR spectrum with a peak at 127.6±0.2 ppm. In some embodiments, crystalline Compound I L-lysine cocrystal is characterized as having a ¹³C SSNMR spectrum with a peak at 126.8±0.2 ppm. In some embodiments, crystalline Compound I L-lysine cocrystal is characterized as having a ¹³C SSNMR spectrum with a peak at 125.8±0.2 ppm. In some embodiments, crystalline Compound I L-lysine cocrystal is characterized as having a ¹³C SSNMR spectrum with a peak at 124.1±0.2 ppm. In some embodiments, crystalline Compound I L-lysine cocrystal is characterized as having a ¹³C SSNMR spectrum with a peak at 121.4±0.2 ppm. In some embodiments, crystalline Compound I L-lysine cocrystal is characterized as having a ¹³C SSNMR spectrum with a peak at 119.6±0.2 ppm. In some embodiments, crystalline Compound I L-lysine cocrystal is characterized as having a ¹³C SSNMR spectrum with a peak at 118.0±0.2 ppm. In some embodiments, crystalline Compound I L-lysine cocrystal is characterized as having a ¹³C SSNMR spectrum with a peak at 78.4±0.2 ppm. In some embodiments, crystalline Compound I L-lysine cocrystal is characterized as having a ¹³C SSNMR spectrum with a peak at 77.1±0.2 ppm. In some embodiments, crystalline Compound I L-lysine cocrystal is characterized as having a ¹³C SSNMR spectrum with a peak at 75.9±0.2 ppm. In some embodiments, crystalline Compound I L-lysine cocrystal is characterized as having a ¹³C SSNMR spectrum with a peak at 73.1±0.2 ppm. In some embodiments, crystalline Compound I L-lysine cocrystal is characterized as having a ¹³C SSNMR spectrum with a peak at 56.8±0.2 ppm. In some embodiments, crystalline Compound I L-lysine cocrystal is characterized as having a ¹³C SSNMR spectrum with a peak at 54.9±0.2 ppm. In some embodiments, crystalline Compound I L-lysine cocrystal is characterized as having a ¹³C SSNMR spectrum with a peak at 45.1±0.2 ppm. In some embodiments, crystalline Compound I L-lysine cocrystal is characterized as having a ¹³C SSNMR spectrum with a peak at 43.6±0.2 ppm. In some embodiments, crystalline Compound I L-lysine cocrystal is characterized as having a ¹³C SSNMR spectrum with a peak at 41.4±0.2 ppm. In some embodiments, crystalline Compound I L-lysine cocrystal is characterized as having a ¹³C SSNMR spectrum with a peak at 39.6±0.2 ppm. In some embodiments, crystalline Compound I L-lysine cocrystal is characterized as having a ¹³C SSNMR spectrum with a peak at 38.8±0.2 ppm. In some embodiments, crystalline Compound I L-lysine cocrystal is characterized as having a ¹³C SSNMR spectrum with a peak at 37.0±0.2 ppm. In some embodiments, crystalline Compound I L-lysine cocrystal is characterized as having a ¹³C SSNMR spectrum with a peak at 34.3±0.2 ppm. In some embodiments, crystalline Compound I L-lysine cocrystal is characterized as having a ¹³C SSNMR spectrum with a peak at 33.4±0.2 ppm. In some embodiments, crystalline Compound I L-lysine cocrystal is characterized as having a ¹³C SSNMR spectrum with a peak at 32.2±0.2 ppm. In some embodiments, crystalline Compound I L-lysine cocrystal is characterized as having a ¹³C SSNMR spectrum with a peak at 31.6±0.2 ppm. In some embodiments, crystalline Compound I L-lysine cocrystal is characterized as having a ¹³C SSNMR spectrum with a peak at 30.6±0.2 ppm. In some embodiments, crystalline Compound I L-lysine cocrystal is characterized as having a ¹³C SSNMR spectrum with a peak at 29.2±0.2 ppm. In some embodiments, crystalline Compound I L-lysine cocrystal is characterized as having a ¹³C SSNMR spectrum with a peak at 27.4±0.2 ppm. In some embodiments, crystalline Compound I L-lysine cocrystal is characterized as having a ¹³C SSNMR spectrum with a peak at 25.8±0.2 ppm. In some embodiments, crystalline Compound I L-lysine cocrystal is characterized as having a ¹³C SSNMR spectrum with a peak at 25.1±0.2 ppm. In some embodiments, crystalline Compound I L-lysine cocrystal is characterized as having a ¹³C SSNMR spectrum with a peak at 22.9±0.2 ppm. In some embodiments, crystalline Compound I L-lysine cocrystal is characterized as having a ¹³C SSNMR spectrum with a peak at 22.5±0.2 ppm. In some embodiments, crystalline Compound I L-lysine cocrystal is characterized as having a ¹³C SSNMR spectrum with a peak at 21.7±0.2 ppm. In some embodiments, crystalline Compound I L-lysine cocrystal is characterized as having a ¹³C SSNMR spectrum with a peak at 20.5±0.2 ppm. In some embodiments, crystalline Compound I L-lysine cocrystal is characterized as having a ¹³C SSNMR spectrum with a peak at 19.4±0.2 ppm. In some embodiments, crystalline Compound I L-lysine cocrystal is characterized as having a ¹³C SSNMR spectrum with a peak at 18.6±0.2 ppm.

In some embodiments, crystalline Compound I L-lysine cocrystal is characterized as having a ¹³C SSNMR spectrum with one, two, three, four, five, six, seven, eight, nine, ten, or more peaks selected from 181.6±0.2 ppm, 180.9±0.2 ppm, 177.5±0.2 ppm, 165.4±0.2 ppm, 164.4±0.2 ppm, 163.7±0.2 ppm, 162.7±0.2 ppm, 151.9±0.2 ppm, 150.7±0.2 ppm, 138.9±0.2 ppm, 138.2±0.2 ppm, 127.6±0.2 ppm, 126.8±0.2 ppm, 125.8±0.2 ppm, 124.1±0.2 ppm, 121.4±0.2 ppm, 119.6±0.2 ppm, 118.0±0.2 ppm, 78.4±0.2 ppm, 77.1±0.2 ppm, 75.9±0.2 ppm, 73.1±0.2 ppm, 56.8±0.2 ppm, 54.9±0.2 ppm, 45.1±0.2 ppm, 43.6±0.2 ppm, 41.4±0.2 ppm, 39.6±0.2 ppm, 38.8±0.2 ppm, 37.0±0.2 ppm, 34.3±0.2 ppm, 33.4±0.2 ppm, 32.2±0.2 ppm, 31.6±0.2 ppm, 30.6±0.2 ppm, 29.2±0.2 ppm, 27.4±0.2 ppm, 25.8±0.2 ppm, 25.1±0.2 ppm, 22.9±0.2 ppm, 22.5±0.2 ppm, 21.7±0.2 ppm, 20.5±0.2 ppm, 19.4±0.2 ppm, and 18.6±0.2 ppm. In some embodiments, crystalline Compound I L-lysine cocrystal is characterized as having a ¹³C SSNMR spectrum with peaks at 181.6±0.2 ppm, 180.9±0.2 ppm, 177.5±0.2 ppm, 165.4±0.2 ppm, 164.4±0.2 ppm, 163.7±0.2 ppm, 162.7±0.2 ppm, 151.9±0.2 ppm, 150.7±0.2 ppm, 138.9±0.2 ppm, 138.2±0.2 ppm, 127.6±0.2 ppm, 126.8±0.2 ppm, 125.8±0.2 ppm, 124.1±0.2 ppm, 121.4±0.2 ppm, 119.6±0.2 ppm, 118.0±0.2 ppm, 78.4±0.2 ppm, 77.1±0.2 ppm, 75.9±0.2 ppm, 73.1±0.2 ppm, 56.8±0.2 ppm, 54.9±0.2 ppm, 45.1±0.2 ppm, 43.6±0.2 ppm, 41.4±0.2 ppm, 39.6±0.2 ppm, 38.8±0.2 ppm, 37.0±0.2 ppm, 34.3±0.2 ppm, 33.4±0.2 ppm, 32.2±0.2 ppm, 31.6±0.2 ppm, 30.6±0.2 ppm, 29.2±0.2 ppm, 27.4±0.2 ppm, 25.8±0.2 ppm, 25.1±0.2 ppm, 22.9±0.2 ppm, 22.5±0.2 ppm, 21.7±0.2 ppm, 20.5±0.2 ppm, 19.4±0.2 ppm, and 18.6±0.2 ppm.

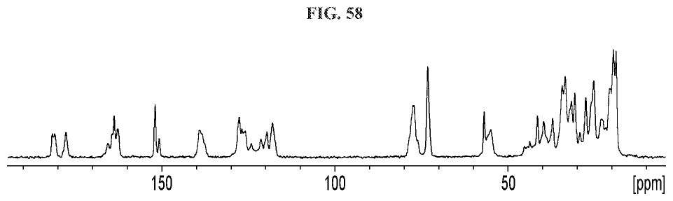

In some embodiments, crystalline Compound I L-lysine cocrystal is characterized by a ¹³C SSNMR spectrum substantially similar to FIG. 58.

Another aspect of the invention provides a method of making crystalline Compound I L-lysine cocrystal. In some embodiments, the method of making crystalline Compound I L-lysine cocrystal comprises: (i) mixing ethanol and water at ratio of 30.8% to 69.2% by volume, (ii) saturating the ethanol/water mixture with L-lysine anhydrate, (iii) saturating the mixture with crystalline Compound I hemihydrate Form C, (iv) adding crystalline Compound I hemihydrate Form C to L-lysine to make a slurry with a 1:1 molar ratio of Compound I to L-lysine, (v) mixing the slurry for 2 days, (vi) sonicating for an additional 3 hours, and (viii) isolating the solids to yield crystalline Compound I L-lysine cocrystal.

### R. Crystalline Compound I L-Arginine Cocrystal

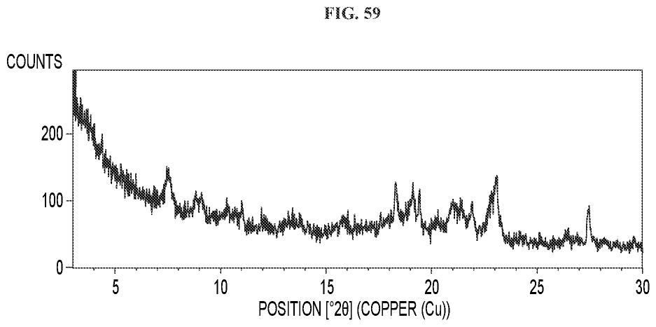

In some embodiments, the invention provides crystalline Compound I L-arginine cocrystal. FIG. 59 provides an X-ray powder diffractogram of crystalline Compound I L-arginine cocrystal.

In some embodiments, crystalline Compound I L-arginine cocrystal is substantially pure. In some embodiments, crystalline Compound I L-arginine cocrystal is substantially crystalline. In some embodiments, crystalline Compound I L-arginine cocrystal is characterized by an X-ray powder diffractogram generated by an X-ray powder diffraction analysis with an incident beam of Cu Kα radiation.

In some embodiments, crystalline Compound I L-arginine cocrystal is characterized by an X-ray powder diffractogram having a signal at 7.5±0.2 degrees two-theta. In some embodiments, crystalline Compound I L-arginine cocrystal is characterized by an X-ray powder diffractogram having a signal at 9.0±0.2 degrees two-theta. In some embodiments, crystalline Compound I L-arginine cocrystal is characterized by an X-ray powder diffractogram having a signal at 10.5±0.2 degrees two-theta. In some embodiments, crystalline Compound I L-arginine cocrystal is characterized by an X-ray powder diffractogram having a signal at 13.4±0.2 degrees two-theta. In some embodiments, crystalline Compound I L-arginine cocrystal is characterized by an X-ray powder diffractogram having a signal at 15.9±0.2 degrees two-theta. In some embodiments, crystalline Compound I L-arginine cocrystal is characterized by an X-ray powder diffractogram having a signal at 18.3±0.2 degrees two-theta. In some embodiments, crystalline Compound I L-arginine cocrystal is characterized by an X-ray powder diffractogram having a signal at 19.1±0.2 degrees two-theta. In some embodiments, crystalline Compound I L-arginine cocrystal is characterized by an X-ray powder diffractogram having a signal at 19.4±0.2 degrees two-theta. In some embodiments, crystalline Compound I L-arginine cocrystal is characterized by an X-ray powder diffractogram having a signal at 21.0±0.2 degrees two-theta. In some embodiments, crystalline Compound I L-arginine cocrystal is characterized by an X-ray powder diffractogram having a signal at 21.9±0.2 degrees two-theta. In some embodiments, crystalline Compound I L-arginine cocrystal is characterized by an X-ray powder diffractogram having a signal at 23.1±0.2 degrees two-theta. In some embodiments, crystalline Compound I L-arginine cocrystal is characterized by an X-ray powder diffractogram having a signal at 27.4±0.2 degrees two-theta.

In some embodiments, crystalline Compound I L-arginine cocrystal is characterized by an X-ray powder diffractogram having a signal at one, two, three, four, or five of 7.5±0.2 degrees two-theta, 18.3±0.2 degrees two-theta, 19.1±0.2 degrees two-theta, 19.4±0.2 degrees two-theta, and 23.1±0.2 degrees two-theta.

In some embodiments, crystalline Compound I L-arginine cocrystal is characterized by an X-ray powder diffractogram having a signal at one, two, three, four, five, six, seven, eight, nine, or ten of 7.5±0.2 degrees two-theta, 9.0±0.2 degrees two-theta, 10.5±0.2 degrees two-theta, 18.3±0.2 degrees two-theta, 19.1±0.2 degrees two-theta, 19.4±0.2 degrees two-theta, 21.0±0.2 degrees two-theta, 21.9±0.2 degrees two-theta, 23.1±0.2 degrees two-theta, and 27.4±0.2 degrees two-theta.

In some embodiments, crystalline Compound I L-arginine cocrystal is characterized by an X-ray powder diffractogram having signals at one, two, three, four, five, six, seven, eight, nine, ten, or more of 7.5±0.2 degrees two-theta, 9.0±0.2 degrees two-theta, 10.5±0.2 degrees two-theta, 13.4±0.2 degrees two-theta, 15.9±0.2 degrees two-theta, 18.3±0.2 degrees two-theta, 19.1±0.2 degrees two-theta, 19.4±0.2 degrees two-theta, 21.0±0.2 degrees two-theta, 21.9±0.2 degrees two-theta, 23.1±0.2 degrees two-theta, and 27.4±0.2 degrees two-theta. In some embodiments, crystalline Compound I L-arginine cocrystal is characterized by an X-ray powder diffractogram having signals at 7.5±0.2 degrees two-theta, 9.0±0.2 degrees two-theta, 10.5±0.2 degrees two-theta, 13.4±0.2 degrees two-theta, 15.9±0.2 degrees two-theta, 18.3±0.2 degrees two-theta, 19.1±0.2 degrees two-theta, 19.4±0.2 degrees two-theta, 21.0±0.2 degrees two-theta, 21.9±0.2 degrees two-theta, 23.1±0.2 degrees two-theta, and 27.4±0.2 degrees two-theta.

In some embodiments, crystalline Compound I L-arginine cocrystal is characterized by an X-ray powder diffractogram substantially similar to FIG. 59.

Another aspect of the invention provides a method of making crystalline Compound I L-arginine cocrystal. In some embodiments, the method of making crystalline Compound I L-arginine cocrystal comprises: (i) preparing a 1:1 molar ratio of crystalline Compound I hemihydrate Form C and L-arginine, (ii) adding ethanol/water (30.8% to 69.2% ethanol:water by volume), (iii) ball milling the mixture at 7500 RPM for 60 seconds with 10 second pauses for 5 cycles, and (iv) drying the solids in a vacuum oven at 45° C. overnight to yield crystalline Compound I L-arginine cocrystal.

### S. Crystalline Compound I L-Phenylalanine Cocrystal

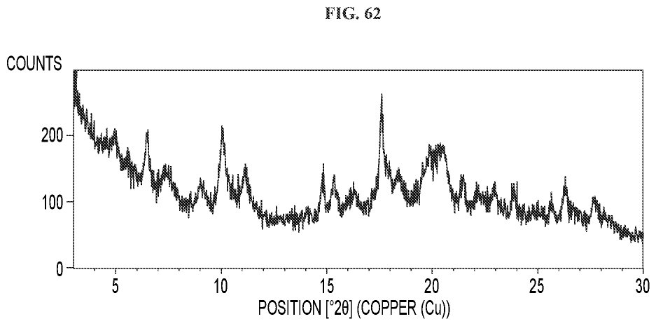

In some embodiments, the invention provides crystalline Compound I L-phenylalanine cocrystal. FIG. 62 provides an X-ray powder diffractogram of crystalline Compound I L-phenylalanine cocrystal.

In some embodiments, crystalline Compound I L-phenylalanine cocrystal is substantially pure. In some embodiments, crystalline Compound I L-phenylalanine cocrystal is substantially crystalline. In some embodiments, crystalline Compound I L-phenylalanine cocrystal is characterized by an X-ray powder diffractogram generated by an X-ray powder diffraction analysis with an incident beam of Cu Kα radiation.

In some embodiments, crystalline Compound I L-phenylalanine cocrystal is characterized by an X-ray powder diffractogram having a signal at 4.9±0.2 degrees two-theta. In some embodiments, crystalline Compound I L-phenylalanine cocrystal is characterized by an X-ray powder diffractogram having a signal at 6.5±0.2 degrees two-theta. In some embodiments, crystalline Compound I L-phenylalanine cocrystal is characterized by an X-ray powder diffractogram having a signal at 7.4±0.2 degrees two-theta. In some embodiments, crystalline Compound I L-phenylalanine cocrystal is characterized by an X-ray powder diffractogram having a signal at 9.0±0.2 degrees two-theta. In some embodiments, crystalline Compound I L-phenylalanine cocrystal is characterized by an X-ray powder diffractogram having a signal at 10.1±0.2 degrees two-theta. In some embodiments, crystalline Compound I L-phenylalanine cocrystal is characterized by an X-ray powder diffractogram having a signal at 11.1±0.2 degrees two-theta. In some embodiments, crystalline Compound I L-phenylalanine cocrystal is characterized by an X-ray powder diffractogram having a signal at 14.8±0.2 degrees two-theta. In some embodiments, crystalline Compound I L-phenylalanine cocrystal is characterized by an X-ray powder diffractogram having a signal at 15.3±0.2 degrees two-theta. In some embodiments, crystalline Compound I L-phenylalanine cocrystal is characterized by an X-ray powder diffractogram having a signal at 16.2±0.2 degrees two-theta. In some embodiments, crystalline Compound I L-phenylalanine cocrystal is characterized by an X-ray powder diffractogram having a signal at 17.6±0.2 degrees two-theta. In some embodiments, crystalline Compound I L-phenylalanine cocrystal is characterized by an X-ray powder diffractogram having a signal at 18.4±0.2 degrees two-theta. In some embodiments, crystalline Compound I L-phenylalanine cocrystal is characterized by an X-ray powder diffractogram having a signal at 19.8±0.2 degrees two-theta. In some embodiments, crystalline Compound I L-phenylalanine cocrystal is characterized by an X-ray powder diffractogram having a signal at 20.5±0.2 degrees two-theta. In some embodiments, crystalline Compound I L-phenylalanine cocrystal is characterized by an X-ray powder diffractogram having a signal at 21.4±0.2 degrees two-theta. In some embodiments, crystalline Compound I L-phenylalanine cocrystal is characterized by an X-ray powder diffractogram having a signal at 22.2±0.2 degrees two-theta. In some embodiments, crystalline Compound I L-phenylalanine cocrystal is characterized by an X-ray powder diffractogram having a signal at 22.9±0.2 degrees two-theta. In some embodiments, crystalline Compound I L-phenylalanine cocrystal is characterized by an X-ray powder diffractogram having a signal at 23.9±0.2 degrees two-theta. In some embodiments, crystalline Compound I L-phenylalanine cocrystal is characterized by an X-ray powder diffractogram having a signal at 26.3±0.2 degrees two-theta. In some embodiments, crystalline Compound I L-phenylalanine cocrystal is characterized by an X-ray powder diffractogram having a signal at 27.9±0.2 degrees two-theta.

In some embodiments, crystalline Compound I L-phenylalanine cocrystal is characterized by an X-ray powder diffractogram having a signal at one, two, three, four, or five of 6.5±0.2 degrees two-theta, 10.1±0.2 degrees two-theta, 11.1±0.2 degrees two-theta, 17.6±0.2 degrees two-theta, and 20.5±0.2 degrees two-theta.

In some embodiments, crystalline Compound I L-phenylalanine cocrystal is characterized by an X-ray powder diffractogram having a signal at one, two, three, four, five, six, seven, eight, nine, or ten of 6.5±0.2 degrees two-theta, 10.1±0.2 degrees two-theta, 11.1±0.2 degrees two-theta, 14.8±0.2 degrees two-theta, 15.3±0.2 degrees two-theta, 17.6±0.2 degrees two-theta, 18.4±0.2 degrees two-theta, 19.8±0.2 degrees two-theta, 20.5±0.2 degrees two-theta, and 21.4±0.2 degrees two-theta.

In some embodiments, crystalline Compound I L-phenylalanine cocrystal is characterized by an X-ray powder diffractogram having a signal at one, two, three, four, five, six, seven, eight, nine, ten, or more of 4.9±0.2 degrees two-theta, 6.5±0.2 degrees two-theta, 7.4±0.2 degrees two-theta, 9.0±0.2 degrees two-theta, 10.1±0.2 degrees two-theta, 11.1±0.2 degrees two-theta, 14.8±0.2 degrees two-theta, 15.3±0.2 degrees two-theta, 16.2±0.2 degrees two-theta, 17.6±0.2 degrees two-theta, 18.4±0.2 degrees two-theta, 19.8±0.2 degrees two-theta, 20.5±0.2 degrees two-theta, 21.4±0.2 degrees two-theta, 22.2±0.2 degrees two-theta, 22.9±0.2 degrees two-theta, 23.9±0.2 degrees two-theta, 26.3±0.2 degrees two-theta, and 27.9±0.2 degrees two-theta. In some embodiments, crystalline Compound I L-phenylalanine cocrystal is characterized by an X-ray powder diffractogram having signals at 4.9±0.2 degrees two-theta, 6.5±0.2 degrees two-theta, 7.4±0.2 degrees two-theta, 9.0±0.2 degrees two-theta, 10.1±0.2 degrees two-theta, 11.1±0.2 degrees two-theta, 14.8±0.2 degrees two-theta, 15.3±0.2 degrees two-theta, 16.2±0.2 degrees two-theta, 17.6±0.2 degrees two-theta, 18.4±0.2 degrees two-theta, 19.8±0.2 degrees two-theta, 20.5±0.2 degrees two-theta, 21.4±0.2 degrees two-theta, 22.2±0.2 degrees two-theta, 22.9±0.2 degrees two-theta, 23.9±0.2 degrees two-theta, 26.3±0.2 degrees two-theta, and 27.9±0.2 degrees two-theta.

In some embodiments, crystalline Compound I L-phenylalanine cocrystal is characterized by an X-ray powder diffractogram substantially similar to FIG. 62.

Another aspect of the invention provides a method of making crystalline Compound I L-phenylalanine cocrystal. In some embodiments, the method of making crystalline Compound I L-phenylalanine cocrystal comprises: (i) preparing a 1:1 molar ratio of crystalline Compound I hemihydrate Form C and L-phenylalanine, (ii) adding ethanol/water (30.8% to 69.2% ethanol:water by volume), (iii) ball milling the mixture at 7500 RPM for 60 seconds with 10 second pauses for 5 cycles, and (iv) drying the solids in a vacuum oven at 45° C. overnight to yield crystalline Compound I L-phenylalanine cocrystal.

### T. Crystalline Compound I Succinic Acid Cocrystal (Wet)

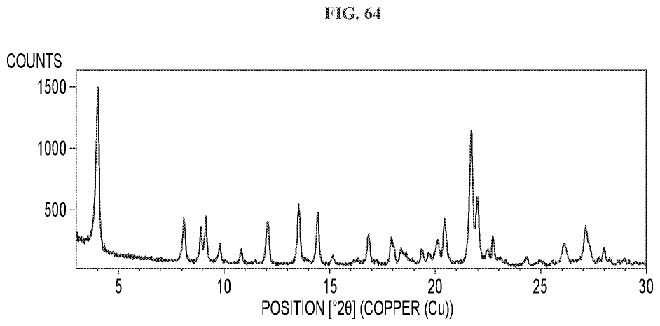

In some embodiments, the invention provides crystalline Compound I succinic acid cocrystal (wet). FIG. 64 provides an X-ray powder diffractogram of crystalline Compound I succinic acid cocrystal (wet).

In some embodiments, crystalline Compound I succinic acid cocrystal (wet) is substantially pure. In some embodiments, crystalline Compound I succinic acid cocrystal (wet) is substantially crystalline. In some embodiments, crystalline Compound I succinic acid cocrystal (wet) is characterized by an X-ray powder diffractogram generated by an X-ray powder diffraction analysis with an incident beam of Cu Ku radiation.

In some embodiments, crystalline Compound I succinic acid cocrystal (wet) is characterized by an X-ray powder diffractogram having (a) a signal at 22.7±0.2 degrees two-theta, and (b) a signal at one, two, three, four, five, six, seven, eight, nine, ten, or more of 4.0±0.2 degrees two-theta, 8.1±0.2 degrees two-theta, 8.9±0.2 degrees two-theta, 9.1±0.2 degrees two-theta, 9.8±0.2 degrees two-theta, 12.1±0.2 degrees two-theta, 13.5±0.2 degrees two-theta, 14.4±0.2 degrees two-theta, 16.8±0.2 degrees two-theta, 17.9±0.2 degrees two-theta, 20.1±0.2 degrees two-theta, 20.4±0.2 degrees two-theta, 21.7±0.2 degrees two-theta, 22.0±0.2 degrees two-theta, 26.1±0.2 degrees two-theta, 27.1±0.2 degrees two-theta, and 28.0±0.2 degrees two-theta.

In some embodiments, crystalline Compound I succinic acid cocrystal (wet) is characterized by an X-ray powder diffractogram having signals at 4.0±0.2 degrees two-theta, 8.1±0.2 degrees two-theta, 8.9±0.2 degrees two-theta, 9.1±0.2 degrees two-theta, 9.8±0.2 degrees two-theta, 12.1±0.2 degrees two-theta, 13.5±0.2 degrees two-theta, 14.4±0.2 degrees two-theta, 16.8±0.2 degrees two-theta, 17.9±0.2 degrees two-theta, 20.1±0.2 degrees two-theta, 20.4±0.2 degrees two-theta, 21.7±0.2 degrees two-theta, 22.0±0.2 degrees two-theta, 22.7±0.2 degrees two-theta, 26.1±0.2 degrees two-theta, 27.1±0.2 degrees two-theta, and 28.0±0.2 degrees two-theta.

In some embodiments, crystalline Compound I succinic acid cocrystal (wet) is characterized by an X-ray powder diffractogram substantially similar to FIG. 64.

Another aspect of the invention provides a method of making crystalline Compound I succinic acid cocrystal (wet). In some embodiments, the method of making crystalline Compound I succinic acid cocrystal (wet) comprises: (i) preparing a 1:1 molar ratio of crystalline Compound I hemihydrate Form C and succinic acid, (ii) adding ethanol/water (30.8% to 69.2% ethanol:water by volume), (iii) ball milling the mixture at 7500 RPM for 60 seconds with 10 second pauses for 5 cycles, (iv) drying the solids in a vacuum oven at 45° C. overnight, and (v) placing the solids in a humidity chamber at 40° C., 75% Relative Humidity to yield crystalline Compound I succinic acid cocrystal hydrate.

### U. Crystalline Compound I Succinic Acid Cocrystal (Dry)

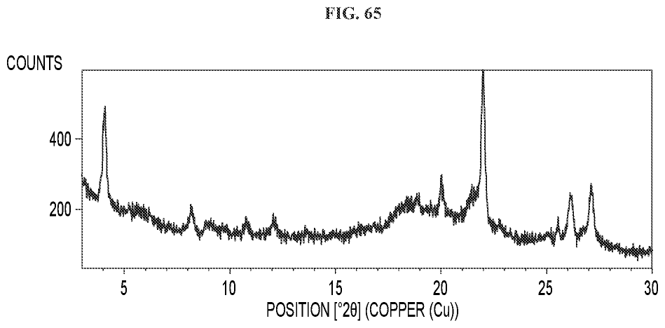

In some embodiments, the invention provides crystalline Compound I succinic acid cocrystal (dry). FIG. 65 provides an X-ray powder diffractogram of crystalline Compound I succinic acid cocrystal (dry).

In some embodiments, crystalline Compound I succinic acid cocrystal (dry) is substantially pure. In some embodiments, crystalline Compound I succinic acid cocrystal (dry) is substantially crystalline. In some embodiments, crystalline Compound I succinic acid cocrystal (dry) is characterized by an X-ray powder diffractogram generated by an X-ray powder diffraction analysis with an incident beam of Cu Ku radiation.

In some embodiments, crystalline Compound I succinic acid cocrystal (dry) is characterized by an X-ray powder diffractogram having (a) a signal at 25.5±0.2 degrees two-theta, and (b) a signal at one, two, three, four, five, six, seven, eight, nine, ten, or more of 4.1±0.2 degrees two-theta, 8.2±0.2 degrees two-theta, 20.0±0.2 degrees two-theta, 22.0±0.2 degrees two-theta, 26.1±0.2 degrees two-theta, and 27.1±0.2 degrees two-theta.

In some embodiments, crystalline Compound I succinic acid cocrystal (dry) is characterized by an X-ray powder diffractogram having signals at 4.1±0.2 degrees two-theta, 8.2±0.2 degrees two-theta, 20.0±0.2 degrees two-theta, 22.0±0.2 degrees two-theta, 25.5±0.2 degrees two-theta, 26.1±0.2 degrees two-theta, and 27.1±0.2 degrees two-theta.

In some embodiments, crystalline Compound I succinic acid cocrystal (dry) is characterized by an X-ray powder diffractogram substantially similar to FIG. 65.

Another aspect of the invention provides a method of making crystalline Compound I succinic acid cocrystal (dry). In some embodiments, the method of making crystalline Compound I succinic acid cocrystal (dry) comprises: (i) preparing a 1:1 molar ratio of crystalline Compound I hemihydrate Form C and succinic acid, (ii) adding ethanol/water (30.8% to 69.2% ethanol:water by volume), (iii) ball milling the mixture at 7500 RPM for 60 seconds with 10 second pauses for 5 cycles, and (iv) drying the solids in a vacuum oven at 45° C. overnight to yield crystalline Compound I succinic acid cocrystal (dry).

### V. Crystalline Compound I Methanol Solvate/Hydrate

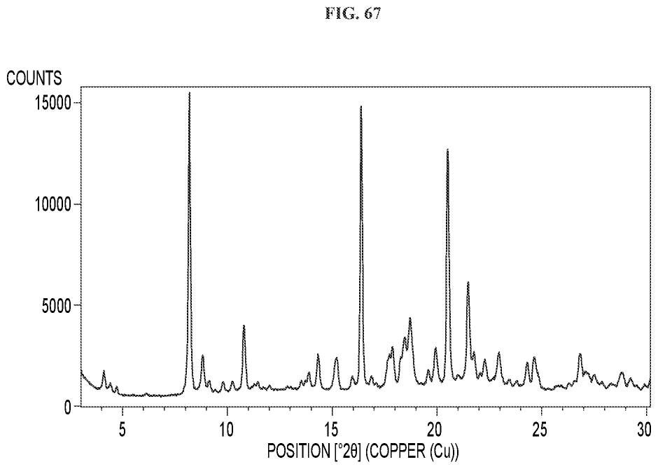

In some embodiments, the invention provides crystalline Compound I methanol solvate/hydrate. FIG. 67 provides an X-ray powder diffractogram of crystalline Compound I methanol solvate/hydrate.

In some embodiments, crystalline Compound I methanol solvate/hydrate is substantially pure. In some embodiments, crystalline Compound I methanol solvate/hydrate is substantially crystalline. In some embodiments, crystalline Compound I methanol solvate/hydrate is characterized by an X-ray powder diffractogram generated by an X-ray powder diffraction analysis with an incident beam of Cu Kα radiation.

In some embodiments, crystalline Compound I methanol solvate/hydrate is characterized by an X-ray powder diffractogram having a signal at 8.2±0.2 degrees two-theta. In some embodiments, crystalline Compound I methanol solvate/hydrate is characterized by an X-ray powder diffractogram having a signal at 8.8±0.2 degrees two-theta. In some embodiments, crystalline Compound I methanol solvate/hydrate is characterized by an X-ray powder diffractogram having a signal at 10.8±0.2 degrees two-theta. In some embodiments, crystalline Compound I methanol solvate/hydrate is characterized by an X-ray powder diffractogram having a signal at 14.3±0.2 degrees two-theta. In some embodiments, crystalline Compound I methanol solvate/hydrate is characterized by an X-ray powder diffractogram having a signal at 16.4±0.2 degrees two-theta. In some embodiments, crystalline Compound I methanol solvate/hydrate is characterized by an X-ray powder diffractogram having a signal at 17.9±0.2 degrees two-theta. In some embodiments, crystalline Compound I methanol solvate/hydrate is characterized by an X-ray powder diffractogram having a signal at 18.5±0.2 degrees two-theta. In some embodiments, crystalline Compound I methanol solvate/hydrate is characterized by an X-ray powder diffractogram having a signal at 18.7±0.2 degrees two-theta. In some embodiments, crystalline Compound I methanol solvate/hydrate is characterized by an X-ray powder diffractogram having a signal at 20.0±0.2 degrees two-theta. In some embodiments, crystalline Compound I methanol solvate/hydrate is characterized by an X-ray powder diffractogram having a signal at 20.5±0.2 degrees two-theta. In some embodiments, crystalline Compound I methanol solvate/hydrate is characterized by an X-ray powder diffractogram having a signal at 21.5±0.2 degrees two-theta. In some embodiments, crystalline Compound I methanol solvate/hydrate is characterized by an X-ray powder diffractogram having a signal at 26.9±0.2 degrees two-theta.

In some embodiments, crystalline Compound I methanol solvate/hydrate is characterized by an X-ray powder diffractogram having a signal at one, two, three, four, or five of 8.2±0.2 degrees two-theta, 16.4±0.2 degrees two-theta, 18.7±0.2 degrees two-theta, 20.5±0.2 degrees two-theta, and 21.5±0.2 degrees two-theta.

In some embodiments, crystalline Compound I methanol solvate/hydrate is characterized by an X-ray powder diffractogram having a signal at one, two, three, four, five, six, seven, eight, nine, or ten of 8.2±0.2 degrees two-theta, 8.8±0.2 degrees two-theta, 10.8±0.2 degrees two-theta, 14.3±0.2 degrees two-theta, 16.4±0.2 degrees two-theta, 17.9±0.2 degrees two-theta, 18.5±0.2 degrees two-theta, 18.7±0.2 degrees two-theta, 20.5±0.2 degrees two-theta, and 21.5±0.2 degrees two-theta.

In some embodiments, crystalline Compound I methanol solvate/hydrate is characterized by an X-ray powder diffractogram having a signal at one, two, three, four, five, six, seven, eight, nine, ten, or more of 8.2±0.2 degrees two-theta, 8.8±0.2 degrees two-theta, 10.8±0.2 degrees two-theta, 14.3±0.2 degrees two-theta, 16.4±0.2 degrees two-theta, 17.9±0.2 degrees two-theta, 18.5±0.2 degrees two-theta, 18.7±0.2 degrees two-theta, 20.0±0.2 degrees two-theta, 20.5±0.2 degrees two-theta, 21.5±0.2 degrees two-theta, and 26.9±0.2 degrees two-theta. In some embodiments, crystalline Compound I methanol solvate/hydrate is characterized by an X-ray powder diffractogram having signals at 8.2±0.2 degrees two-theta, 8.8±0.2 degrees two-theta, 10.8±0.2 degrees two-theta, 14.3±0.2 degrees two-theta, 16.4±0.2 degrees two-theta, 17.9±0.2 degrees two-theta, 18.5±0.2 degrees two-theta, 18.7±0.2 degrees two-theta, 20.0±0.2 degrees two-theta, 20.5±0.2 degrees two-theta, 21.5±0.2 degrees two-theta, and 26.9±0.2 degrees two-theta.

In some embodiments, crystalline Compound I methanol solvate/hydrate is characterized by an X-ray powder diffractogram substantially similar to FIG. 67.

In some embodiments, crystalline Compound I methanol solvate/hydrate is characterized as having a ¹³C SSNMR spectrum with a peak at 163.3±0.2 ppm. In some embodiments, crystalline Compound I methanol solvate/hydrate is characterized as having a ¹³C SSNMR spectrum with a peak at 162.2±0.2 ppm. In some embodiments, crystalline Compound I methanol solvate/hydrate is characterized as having a ¹³C SSNMR spectrum with a peak at 151.6±0.2 ppm. In some embodiments, crystalline Compound I methanol solvate/hydrate is characterized as having a ¹³C SSNMR spectrum with a peak at 150.8±0.2 ppm. In some embodiments, crystalline Compound I methanol solvate/hydrate is characterized as having a ¹³C SSNMR spectrum with a peak at 138.4±0.2 ppm. In some embodiments, crystalline Compound I methanol solvate/hydrate is characterized as having a ¹³C SSNMR spectrum with a peak at 126.4±0.2 ppm. In some embodiments, crystalline Compound I methanol solvate/hydrate is characterized as having a ¹³C SSNMR spectrum with a peak at 125.4±0.2 ppm. In some embodiments, crystalline Compound I methanol solvate/hydrate is characterized as having a ¹³C SSNMR spectrum with a peak at 122.3±0.2 ppm. In some embodiments, crystalline Compound I methanol solvate/hydrate is characterized as having a ¹³C SSNMR spectrum with a peak at 121.5±0.2 ppm. In some embodiments, crystalline Compound I methanol solvate/hydrate is characterized as having a ¹³C SSNMR spectrum with a peak at 120.7±0.2 ppm. In some embodiments, crystalline Compound I methanol solvate/hydrate is characterized as having a ¹³C SSNMR spectrum with a peak at 118.9±0.2 ppm. In some embodiments, crystalline Compound I methanol solvate/hydrate is characterized as having a ¹³C SSNMR spectrum with a peak at 118.2±0.2 ppm. In some embodiments, crystalline Compound I methanol solvate/hydrate is characterized as having a ¹³C SSNMR spectrum with a peak at 117.4±0.2 ppm. In some embodiments, crystalline Compound I methanol solvate/hydrate is characterized as having a ¹³C SSNMR spectrum with a peak at 77.5±0.2 ppm. In some embodiments, crystalline Compound I methanol solvate/hydrate is characterized as having a ¹³C SSNMR spectrum with a peak at 73.2±0.2 ppm. In some embodiments, crystalline Compound I methanol solvate/hydrate is characterized as having a ¹³C SSNMR spectrum with a peak at 49.4±0.2 ppm. In some embodiments, crystalline Compound I methanol solvate/hydrate is characterized as having a ¹³C SSNMR spectrum with a peak at 36.5±0.2 ppm. In some embodiments, crystalline Compound I methanol solvate/hydrate is characterized as having a ¹³C SSNMR spectrum with a peak at 35.2±0.2 ppm. In some embodiments, crystalline Compound I methanol solvate/hydrate is characterized as having a ¹³C SSNMR spectrum with a peak at 34.1±0.2 ppm. In some embodiments, crystalline Compound I methanol solvate/hydrate is characterized as having a ¹³C SSNMR spectrum with a peak at 33.5±0.2 ppm. In some embodiments, crystalline Compound I methanol solvate/hydrate is characterized as having a ¹³C SSNMR spectrum with a peak at 32.5±0.2 ppm. In some embodiments, crystalline Compound I methanol solvate/hydrate is characterized as having a ¹³C SSNMR spectrum with a peak at 25.4±0.2 ppm. In some embodiments, crystalline Compound I methanol solvate/hydrate is characterized as having a ¹³C SSNMR spectrum with a peak at 22.8±0.2 ppm. In some embodiments, crystalline Compound I methanol solvate/hydrate is characterized as having a ¹³C SSNMR spectrum with a peak at 22.1±0.2 ppm. In some embodiments, crystalline Compound I methanol solvate/hydrate is characterized as having a ¹³C SSNMR spectrum with a peak at 21.4±0.2 ppm. In some embodiments, crystalline Compound I methanol solvate/hydrate is characterized as having a ¹³C SSNMR spectrum with a peak at 20.5±0.2 ppm. In some embodiments, crystalline Compound I methanol solvate/hydrate is characterized as having a ¹³C SSNMR spectrum with a peak at 20.0±0.2 ppm. In some embodiments, crystalline Compound I methanol solvate/hydrate is characterized as having a ¹³C SSNMR spectrum with a peak at 19.5±0.2 ppm.

In some embodiments, crystalline Compound I methanol solvate/hydrate is characterized as having a ¹³C SSNMR spectrum with one, two, three, four, five, six, seven, eight, nine, ten, or more peaks selected from 163.3±0.2 ppm, 162.2±0.2 ppm, 151.6±0.2 ppm, 150.8±0.2 ppm, 138.4±0.2 ppm, 126.4±0.2 ppm, 125.4±0.2 ppm, 122.3±0.2 ppm, 121.5±0.2 ppm, 120.7±0.2 ppm, 118.9±0.2 ppm, 118.2±0.2 ppm, 117.4±0.2 ppm, 77.5±0.2 ppm, 73.2±0.2 ppm, 49.4±0.2 ppm, 36.5±0.2 ppm, 35.2±0.2 ppm, 34.1±0.2 ppm, 33.5±0.2 ppm, 32.5±0.2 ppm, 25.4±0.2 ppm, 22.8±0.2 ppm, 22.1±0.2 ppm, 21.4±0.2 ppm, 20.5±0.2 ppm, 20.0±0.2 ppm, and 19.5±0.2 ppm. In some embodiments, crystalline Compound I methanol solvate/hydrate is characterized as having a ¹³C SSNMR spectrum with peaks at 163.3±0.2 ppm, 162.2±0.2 ppm, 151.6±0.2 ppm, 150.8±0.2 ppm, 138.4±0.2 ppm, 126.4±0.2 ppm, 125.4±0.2 ppm, 122.3±0.2 ppm, 121.5±0.2 ppm, 120.7±0.2 ppm, 118.9±0.2 ppm, 118.2±0.2 ppm, 117.4±0.2 ppm, 77.5±0.2 ppm, 73.2±0.2 ppm, 49.4±0.2 ppm, 36.5±0.2 ppm, 35.2±0.2 ppm, 34.1±0.2 ppm, 33.5±0.2 ppm, 32.5±0.2 ppm, 25.4±0.2 ppm, 22.8±0.2 ppm, 22.1±0.2 ppm, 21.4±0.2 ppm, 20.5±0.2 ppm, 20.0±0.2 ppm, and 19.5±0.2 ppm.

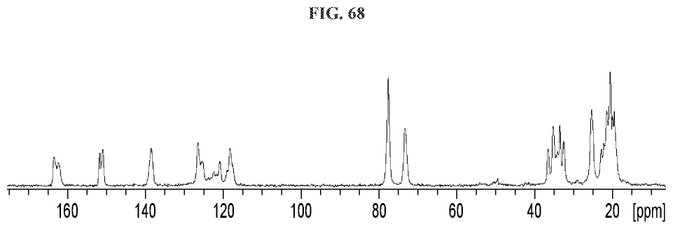

In some embodiments, crystalline Compound I methanol solvate/hydrate is characterized by a ¹³C SSNMR spectrum substantially similar to FIG. 68.

In some embodiments, crystalline Compound I methanol solvate/hydrate is characterized as having a ¹⁹F SSNMR spectrum with a peak at −64.0±0.2 ppm. In some embodiments, crystalline Compound I methanol solvate/hydrate is characterized as having a ¹⁹F SSNMR spectrum with a peak at −64.6±0.2 ppm. In some embodiments, crystalline Compound I methanol solvate/hydrate is characterized as having a ¹⁹F SSNMR spectrum with a peak at −79.0±0.2 ppm.

In some embodiments, crystalline Compound I methanol solvate/hydrate is characterized as having a ¹⁹F SSNMR spectrum with one, two, or three peaks at −64.0±0.2 ppm, −64.6±0.2 ppm, and −79.0±0.2 ppm

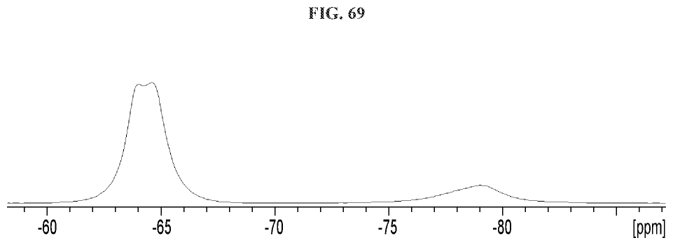

In some embodiments, crystalline Compound I methanol solvate/hydrate is characterized by a ¹⁹F SSNMR spectrum substantially similar to FIG. 69.

In some embodiments, crystalline Compound I methanol solvate/hydrate is characterized by a monoclinic crystal system, C2 space group, and the following unit cell dimensions measured at 100 K on a Bruker diffractometer utilizing Cu K_{α} radiation (λ=1.54178 Å):

| | | | |
|---|---|---|---|
| a | 12.7 ± 0.1 Å | α | 90° |
| b | 15.7 ± 0.1 Å | β | 91.0 ± 0.2° |
| c | 43.0 ± 0.2 Å | γ | 90°. |

Another aspect of the invention provides a method of making crystalline Compound I methanol solvate/hydrate. In some embodiments, the method of making crystalline Compound I methanol solvate/hydrate comprises: (i) combining crystalline Compound I hemihydrate Form C and methanol, (ii) stirring the mixture, and (iii) isolating the solids to yield crystalline Compound I methanol solvate/hydrate.

### W. Methods of Treatment

Compound I, in any one of the pharmaceutically acceptable solid forms disclosed herein, acts as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the invention provides methods of treating, lessening the severity of, or symptomatically treating cystic fibrosis in a patient comprising administering to the patient an effective amount of Compound I in any one of the pharmaceutically acceptable crystalline forms disclosed herein, alone or in combination with another active ingredient, such as another CFTR modulating agent. In some embodiments, the patient has an F508del/minimal function (MF) genotype, F508del/F508del genotype (homozygous for the F508del mutation), F508del/gating genotype, or F508del/residual function (RF) genotype. In some embodiments the patient is heterozygous and has one F508del mutation. In some embodiments the patient is homozygous for the N1303K mutation.

In some embodiments, the patient is heterozygous and has an F508del mutation on one allele and a mutation on the other allele selected from Table 1:

TABLE 1
CFTR Mutations
Mutation

| | | | | |
|---|---|---|---|---|
| Q2X | L218X | Q525X | R792X | E1104X |
| S4X | Q220X | G542X | E822X | W1145X |
| W19X | Y275X | G550X | W882X | R1158X |
| G27X | C276X | Q552X | W846X | R1162X |
| Q39X | Q290X | R553X | Y849X | S1196X |
| W57X | G330X | E585X | R851X | W1204X |
| E60X | W401X | G673X | Q890X | L1254X |
| R75X | Q414X | Q685X | S912X | S1255X |
| L88X | S434X | R709X | Y913X | W1282X |
| E92X | S466X | K710X | Q1042X | Q1313X |
| Q98X | S489X | Q715X | W1089X | Q1330X |
| Y122X | Q493X | L732X | Y1092X | E1371X |
| E193X | W496X | R764X | W1098X | Q1382X |
| W216X | C524X | R785X | R1102X | Q1411X |
| 185 + 1G→T | 711 + 5G→A | 1717 − 8G→A | 2622 + 1G→A | 3 +− 1G→A |
| 296 + 1G→A | 712 − 1G→T | 1717 − 1G→A | 2790 − 1G→C | 3500 − 2A→G |
| 296 + 1G→T | 1248 + 1G→A | 1811 + 1G→C | 3040G→C | 3600 + 2insT |
| 405 + 1G→A | 1249 − 1G→A | 1811 + 1.6kbA→G | (G970R) | 3850 − 1G→A |
| 405 + 3A→C | 1341 + 1G→A | 1811 + 1643G→T | 3120G→A | 4005 + 1G→A |
| 406 − 1G→A | 1525 − 2A→G | 1812 − 1G→A | 3120 + 1G→A | 4374 + 1G→T |
| 621 + 1G→T | 1525 − 1G→A | 1898 + 1G→A | 3121 − 2A→G | |
| 711 + 1G→T | | 1898 + 1G→C | | |
| 182delT | 1078delT | 1677delTA | 2711delT | 3737delA |
| 306insA | 1119delA | 1782delA | 2732insA | 3791delC |
| 306delTAGA | 1138insG | 1824delA | 2869insG | 3821delT |
| 365 − 366insT | 1154insTC | 1833delT | 2896insAG | 3876delA |
| 394delTT | 1161delC | 2043delG | 2942insT | 3878delG |
| 442delA | 1213delT | 2143delT | 2957delT | 3905insT |
| 444delA | 1259insA | 2183AA→G | 3007delG | 4016insT |
| 457TAT→G | 1288insTA | 2184delA | 3028delA | 4021dupT |
| 541delC | 1343delG | 2184insA | 3171delC | 4022insT |
| 574delA | 1471delA | 2307insA | 3171insC | 4040delA |
| 663delT | 1497delGG | 2347delG | 3271delGG | 4279insA |
| 849delG | 1548delG | 2585delT | 3349insT | 4326delTC |
| 935delA | 1609del CA | 2594delGT | 3659delC | |
| CFTRdele1 | | CFTRdele16-17b | 1461ins4 | |
| CFTRdele2 | | CFTRdele17a, 17b | 1924del7 | |
| CFTRdele2, 3 | | CFTRdele17a-18 | 2055del9→A | |
| CFTRdele2-4 | | CFTRdele19 | 2105-2117del13insAGAAA | |
| CFTRdele3-10, 14b-16 | | CFTRdele19-21 | 2372del8 | |
| CFTRdele4-7 | | CFTRdele21 | 2721del11 | |
| CFTRdele4-11 | | CFTRdele22-24 | 2991del32 | |
| CFTR50kbdel | | CFTRdele22, 23 | 3667ins4 | |
| CFTRdup6b-10 | | 124del23bp | 4010del4 | |
| CFTRdele11 | | 602del14 | 4209TGTT→AA | |
| CFTRdele13, 14a | | 852del22 | | |
| CFTRdele14b-17b | | 991del5 | | |
| A46D | V520F | Y569D | N1303K | |
| G85E | A559T | L1065P | | |
| R347P | R560T | R1066C | | |
| L467P | R560S | L1077P | | |
| I507del | A561E | M1101K | | |

In some embodiments, the invention provides methods of treating, lessening the severity of, or symptomatically treating cystic fibrosis in a patient comprising administering to the patient an effective amount of Compound I in any one of the pharmaceutically acceptable solid (e.g., crystalline or amorphous) forms disclosed herein. In some embodiments, the pharmaceutically acceptable solid form of Compound I is a substantially amorphous form. In some embodiments, the pharmaceutically acceptable solid form of Compound I is a substantially crystalline form.

In some embodiments, the method of treating, lessening the severity of, or symptomatically treating cystic fibrosis in a patient comprises administering to the patient an effective amount of Compound I in any one of the pharmaceutically acceptable crystalline forms disclosed herein. In some embodiments, the pharmaceutically acceptable crystalline form of Compound I is Compound I neat Form A. In some embodiments, the pharmaceutically acceptable crystalline form of Compound I is Compound I neat Form B. In some embodiments, the pharmaceutically acceptable crystalline form of Compound I is Compound I hemihydrate Form C. In some embodiments, the pharmaceutically acceptable crystalline form of Compound I is Compound I neat Form D. In some embodiments, the pharmaceutically acceptable crystalline form of Compound I is Compound I neat Form E. In some embodiments, the pharmaceutically acceptable crystalline form of Compound I is Compound I acetic acid solvate. In some embodiments, the pharmaceutically acceptable crystalline form of Compound I is Compound I heptane solvate Form B. In some embodiments, the pharmaceutically acceptable crystalline form of Compound I is Compound I heptane solvate Form C. In some embodiments, the pharmaceutically acceptable crystalline form of Compound I is Compound I octane solvate. In some embodiments, the pharmaceutically acceptable crystalline form of Compound I is Compound I cyclohexane solvate Form A. In some embodiments, the pharmaceutically acceptable crystalline form of Compound I is Compound I cyclohexane solvate Form B. In some embodiments, the pharmaceutically acceptable crystalline form of Compound I is Compound I cyclohexane solvate Form C. In some embodiments, the pharmaceutically acceptable crystalline form of Compound I is Compound I ethanol solvate. In some embodiments, the pharmaceutically acceptable crystalline form of Compound I is Compound I solvate/hydrate (dry). In some embodiments, the pharmaceutically acceptable crystalline form of Compound I is Compound I solvate/hydrate (wet). In some embodiments, the pharmaceutically acceptable crystalline form of Compound I is Compound I L-lysine cocrystal. In some embodiments, the pharmaceutically acceptable crystalline form of Compound I is Compound I L-arginine cocrystal. In some embodiments, the pharmaceutically acceptable crystalline form of Compound I is Compound I L-phenylalanine cocrystal. In some embodiments, the pharmaceutically acceptable crystalline form of Compound I is Compound I succinic acid cocrystal (wet). In some embodiments, the pharmaceutically acceptable crystalline form of Compound I is Compound I succinic acid cocrystal (dry). In some embodiments, the pharmaceutically acceptable crystalline form of Compound I is Compound I methanol solvate/hydrate.

In some embodiments, the method of treating, lessening the severity of, or symptomatically treating cystic fibrosis in a patient comprises administering to the patient an effective amount of Compound I in a pharmaceutically acceptable amorphous form disclosed herein. In some embodiments, the pharmaceutically acceptable crystalline form of Compound I is Compound I neat amorphous form.

In some embodiments, the method of treating, lessening the severity of, or symptomatically treating cystic fibrosis in a patient comprises administering to the patient an effective amount of Compound I as any one of the pharmaceutically acceptable solid (e.g., crystalline or amorphous) forms disclosed herein in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator.

In some embodiments, the method of treating, lessening the severity of, or symptomatically treating cystic fibrosis in a patient comprises administering to the patient an effective amount of Compound I as any one of the pharmaceutically acceptable solid (e.g., crystalline or amorphous) forms disclosed herein in combination with at least one additional active pharmaceutical ingredient. In some embodiments, the at least one additional active pharmaceutical ingredient is selected from Compound II, Compound III, Compound III-d, Compound IV, Compound V, Compound VI, Compound VII, Compound VIII, Compound IX, Compound X, and pharmaceutically acceptable salts and deuterated derivatives thereof.

In some embodiments, the method of treating, lessening the severity of, or symptomatically treating cystic fibrosis in a patient comprises administering to the patient an effective amount of Compound I as a solid form selected from Compound I neat Form A, Compound I neat Form B, Compound I hemihydrate Form C, Compound I neat Form D, Compound I neat Form E, Compound I acetic acid solvate, Compound I heptane solvate Form B, Compound I heptane solvate Form C, Compound I octane solvate, Compound I cyclohexane solvate Form A, Compound I cyclohexane solvate Form B, Compound I cyclohexane solvate Form C, Compound I ethanol solvate, Compound I solvate/hydrate (dry), Compound I solvate/hydrate (wet), Compound I L-lysine cocrystal, Compound I L-arginine cocrystal, Compound I L-phenylalanine cocrystal, Compound I succinic acid cocrystal (wet), Compound I succinic acid cocrystal (dry), Compound I methanol solvate/hydrate, and Compound I neat amorphous form, in combination with at least one additional active pharmaceutical ingredient. In some embodiments, the at least one additional active pharmaceutical ingredient is Compound II, Compound III, Compound III-d, Compound IV, Compound V, Compound VI, Compound VII, Compound VIII, Compound IX, Compound X, and pharmaceutically acceptable salts and deuterated derivatives thereof.

In some embodiments, the method of treating, lessening the severity of, or symptomatically treating cystic fibrosis in a patient comprises administering to the patient an effective amount of Compound I as a solid crystalline form selected from Compound I neat Form A, Compound I neat Form B, Compound I hemihydrate Form C, Compound I neat Form D, Compound I neat Form E, Compound I acetic acid solvate, Compound I heptane solvate Form B, Compound I heptane solvate Form C, Compound I octane solvate, Compound I cyclohexane solvate Form A, Compound I cyclohexane solvate Form B, Compound I cyclohexane solvate Form C, Compound I ethanol solvate, Compound I solvate/hydrate (dry), Compound I solvate/hydrate (wet), Compound I L-lysine cocrystal, Compound I L-arginine cocrystal, Compound I L-phenylalanine cocrystal, Compound I succinic acid cocrystal (wet), Compound I succinic acid cocrystal (dry), and Compound I methanol solvate/hydrate, in combination with at least one additional active pharmaceutical ingredient. In some embodiments, the at least one additional active pharmaceutical ingredient is Compound II, Compound III, Compound III-d, Compound IV, Compound V, Compound VI, Compound VII, Compound VIII, Compound IX, Compound X, and pharmaceutically acceptable salts and deuterated derivatives thereof

In some embodiments, the method of treating, lessening the severity of, or symptomatically treating cystic fibrosis in a patient comprises administering to the patient an effective amount of Compound I as a solid amorphous form that is Compound I neat amorphous form, in combination with at least one additional active pharmaceutical ingredient. In some embodiments, the at least one additional active pharmaceutical ingredient is Compound II, Compound III, Compound III-d, Compound IV, Compound V, Compound VI, Compound VII, Compound VIII, Compound IX, Compound X, and pharmaceutically acceptable salts and deuterated derivatives thereof.

In some embodiments, the method of treating, lessening the severity of, or symptomatically treating cystic fibrosis in a patient comprises administering to the patient (a) an effective amount of Compound I in a solid form selected from Compound I neat amorphous form, Compound I neat Form A, Compound I neat Form B, Compound I hemihydrate Form C, Compound I neat Form D, Compound I neat Form E, Compound I acetic acid solvate, Compound I heptane solvate Form B, Compound I heptane solvate Form C, Compound I octane solvate, Compound I cyclohexane solvate Form A, Compound I cyclohexane solvate Form B, Compound I cyclohexane solvate Form C, Compound I ethanol solvate, Compound I solvate/hydrate (dry), Compound I solvate/hydrate (wet), Compound I L-lysine cocrystal, Compound I L-arginine cocrystal, Compound I L-phenylalanine cocrystal, Compound I succinic acid cocrystal (wet), Compound I succinic acid cocrystal (dry), and Compound I methanol solvate/hydrate, in combination with (b) at least one compound chosen from Compound II, Compound III, Compound III-d, Compound IV, Compound V, Compound VI, Compound VII, Compound VIII, Compound IX, Compound X, and deuterated derivatives and pharmaceutically acceptable salts thereof, and (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; U.S. Provisional Patent Application Nos. 63/328,097 and 63/393,405; Phuan, P.-W. et al. J Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J Cyst. Fibros. 2020, 19 Suppl 1, S25-S32; Crawford, D. K. J Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J of Med. Chem. 2020, 112888.

### X. Pharmaceutical Compositions

Another aspect of the invention provides pharmaceutical compositions comprising Compound I in any one of the pharmaceutically acceptable solid (e.g., crystalline or amorphous) forms disclosed herein. In some embodiments, the pharmaceutical composition comprises Compound I in a solid form selected from Compound I neat Form A, Compound I neat Form B, Compound I hemihydrate Form C, Compound I neat Form D, Compound I neat Form E, Compound I acetic acid solvate, Compound I heptane solvate Form B, Compound I heptane solvate Form C, Compound I octane solvate, Compound I cyclohexane solvate Form A, Compound I cyclohexane solvate Form B, Compound I cyclohexane solvate Form C, Compound I ethanol solvate, Compound I solvate/hydrate (dry), Compound I solvate/hydrate (wet), Compound I L-lysine cocrystal, Compound I L-arginine cocrystal, Compound I L-phenylalanine cocrystal, Compound I succinic acid cocrystal (wet), Compound I succinic acid cocrystal (dry), Compound I methanol solvate/hydrate, and Compound I neat amorphous form. In some embodiments, the pharmaceutical composition comprises Compound I in a solid crystalline form selected from Compound I neat Form A, Compound I neat Form B, Compound I hemihydrate Form C, Compound I neat Form D, Compound I neat Form E, Compound I acetic acid solvate, Compound I heptane solvate Form B, Compound I heptane solvate Form C, Compound I octane solvate, Compound I cyclohexane solvate Form A, Compound I cyclohexane solvate Form B, Compound I cyclohexane solvate Form C, Compound I ethanol solvate, Compound I solvate/hydrate (dry), Compound I solvate/hydrate (wet), Compound I L-lysine cocrystal, Compound I L-arginine cocrystal, Compound I L-phenylalanine cocrystal, Compound I succinic acid cocrystal (wet), Compound I succinic acid cocrystal (dry), and Compound I methanol solvate/hydrate. In some embodiments, the pharmaceutical composition comprises Compound I in a solid amorphous form that is Compound I neat amorphous form.

In some embodiments, the invention provides pharmaceutical compositions comprising Compound I in any one of the pharmaceutically acceptable solid (e.g., crystalline or amorphous) forms disclosed herein in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises Compound I as any one of the pharmaceutically acceptable crystalline forms disclosed herein and at least two additional active pharmaceutical ingredients, one of which is a CFTR corrector and one of which is a CFTR potentiator. In some embodiments, at least one additional active pharmaceutical ingredient is Compound II, Compound III, Compound III-d, Compound IV, Compound V, Compound VI, Compound VII, Compound VIII, Compound IX, Compound X, and pharmaceutically acceptable salts and deuterated derivatives thereof.

In some embodiments, at least one additional active pharmaceutical ingredient is selected from mucolytic agents, bronchodilators, antibiotics, anti-infective agents, and anti-inflammatory agents.

In some embodiments, the invention provides a pharmaceutical composition comprising (a) Compound I in any one of the pharmaceutically acceptable solid (e.g., crystalline or amorphous) forms disclosed herein, and (b) at least one pharmaceutically acceptable carrier.

In some embodiments, the invention provides pharmaceutical compositions comprising (a) Compound I in any one of the pharmaceutically acceptable solid (e.g., crystalline or amorphous) forms disclosed herein, (b) at least one compound chosen from Compound II, Compound III, Compound III-d, Compound IV, Compound V, Compound VI, Compound VII, Compound VIII, Compound IX, Compound X, and pharmaceutically acceptable salts and deuterated derivatives thereof, and (c) at least one pharmaceutically acceptable carrier.

In some embodiments, the invention provides pharmaceutical compositions comprising (a) Compound I in a solid form selected from Compound I neat Form A, Compound I neat Form B, Compound I hemihydrate Form C, Compound I neat Form D, Compound I neat Form E, Compound I acetic acid solvate, Compound I heptane solvate Form B, Compound I heptane solvate Form C, Compound I octane solvate, Compound I cyclohexane solvate Form A, Compound I cyclohexane solvate Form B, Compound I cyclohexane solvate Form C, Compound I ethanol solvate, Compound I solvate/hydrate (dry), Compound I solvate/hydrate (wet), Compound I L-lysine cocrystal, Compound I L-arginine cocrystal, Compound I L-phenylalanine cocrystal, Compound I succinic acid cocrystal (wet), Compound I succinic acid cocrystal (dry), and Compound I methanol solvate/hydrate, (b) at least one compound chosen from Compound II, Compound III, Compound III-d, Compound IV, Compound V, Compound VI, Compound VII, Compound VIII, Compound IX, Compound X, and pharmaceutically acceptable salts and deuterated derivatives thereof, and (c) at least one pharmaceutically acceptable carrier.

In some embodiments, the invention provides pharmaceutical compositions comprising (a) Compound I in a solid form selected from Compound I neat amorphous form, Compound I neat Form A, Compound I neat Form B, Compound I hemihydrate Form C, Compound I neat Form D, Compound I neat Form E, Compound I acetic acid solvate, Compound I heptane solvate Form B, Compound I heptane solvate Form C, Compound I octane solvate, Compound I cyclohexane solvate Form A, Compound I cyclohexane solvate Form B, Compound I cyclohexane solvate Form C, Compound I ethanol solvate, Compound I solvate/hydrate (dry), Compound I solvate/hydrate (wet), Compound I L-lysine cocrystal, Compound I L-arginine cocrystal, Compound I L-phenylalanine cocrystal, Compound I succinic acid cocrystal (wet), Compound I succinic acid cocrystal (dry), and Compound I methanol solvate/hydrate, (b) at least one compound chosen from Compound II, Compound III, Compound III-d, Compound IV, Compound V, Compound VI, Compound VII, Compound VIII, Compound IX, Compound X, and pharmaceutically acceptable salts and deuterated derivatives thereof, (c) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; U.S. Provisional Patent Application Nos. 63/328,097 and 63/393,405; Phuan, P.-W. et al. J Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J Cyst. Fibros. 2020, 19 Suppl 1, S25-S32; Crawford, D. K. J Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J of Med. Chem. 2020, 112888, and (d) at least one pharmaceutically acceptable carrier.

The pharmaceutical compositions described herein are useful for treating cystic fibrosis and other CFTR-mediated diseases.

As described above, pharmaceutical compositions disclosed herein may optionally further comprise at least one pharmaceutically acceptable carrier. The at least one pharmaceutically acceptable carrier may be selected from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York disclose various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Y. Further Compounds of the Disclosure

In some embodiments, Compound I is prepared using a compound of the disclosure.

In some embodiments, Compound I is prepared using a compound selected from:

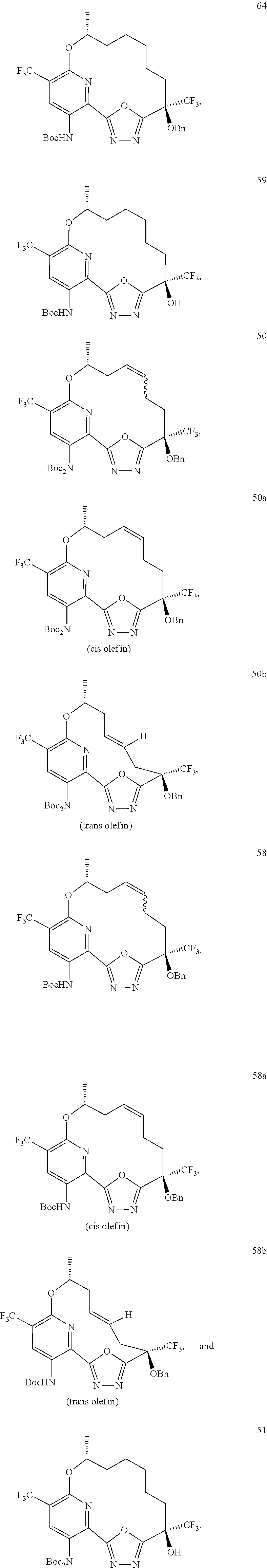

In some embodiments, Compound I is prepared using a compound selected from:

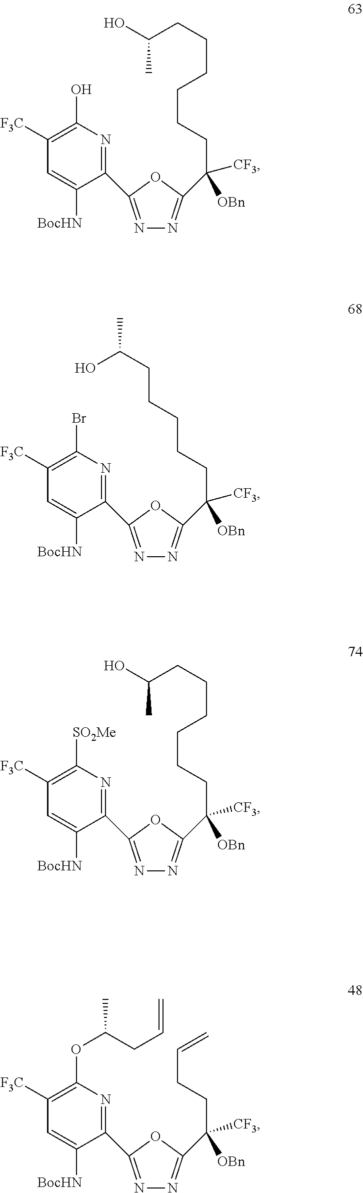 [SMILES: C[C@H](O)CCCCCC[C@@](OCc1ccccc1)(c1nnc(-c2nc(O)c(C(F)(F)F)cc2NC(=O)OC(C)(C)C)o1)C(F)(F)F ; C[C@@H](O)CCCCC[C@@](OCc1ccccc1)(c1nnc(-c2nc(Br)c(C(F)(F)F)cc2NC(=O)OC(C)(C)C)o1)C(F)(F)F ; C[C@@H](O)CCCCCC[C@@](OCc1ccccc1)(c1nnc(-c2nc(S(C)(=O)=O)c(C(F)(F)F)cc2NC(=O)OC(C)(C)C)o1)C(F)(F)F ; C=CCC[C@@](OCc1ccccc1)(c1nnc(-c2nc(O[C@H](C)CC=C)c(C(F)(F)F)cc2NC(=O)OC(C)(C)C)o1)C(F)(F)F]

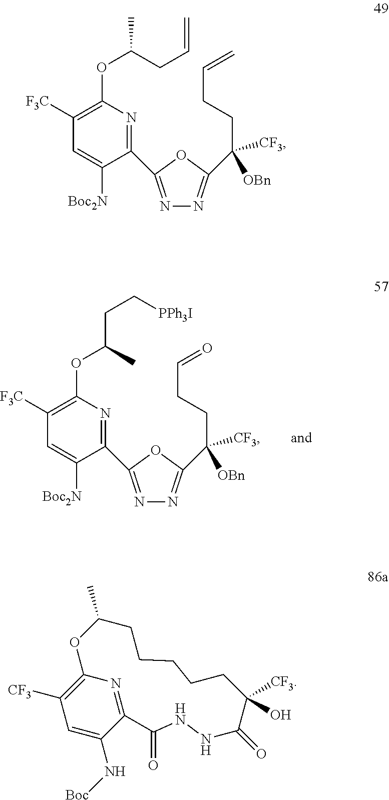

In some embodiments, Compound I is prepared using a compound selected from:

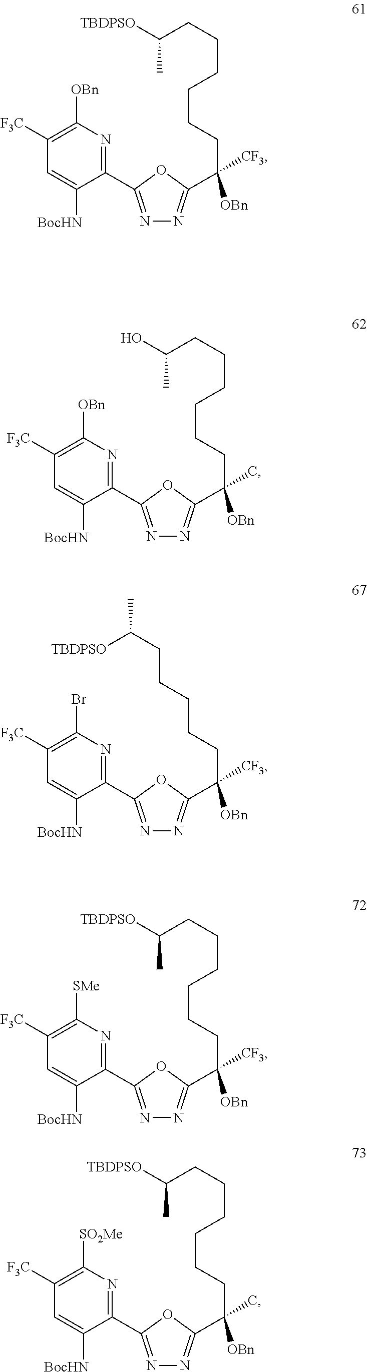

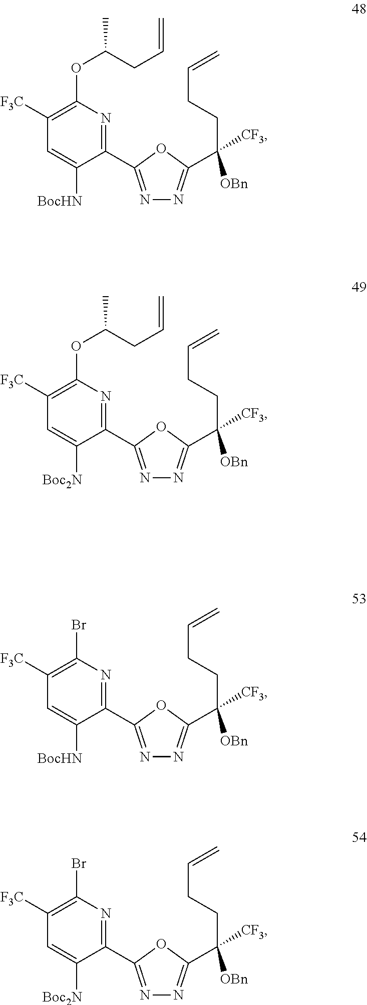

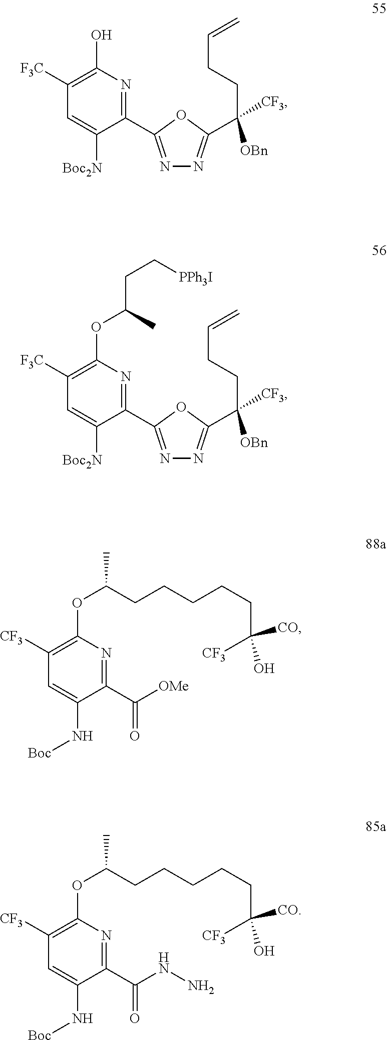

In some embodiments, Compound I can be prepared using a compound selected from:

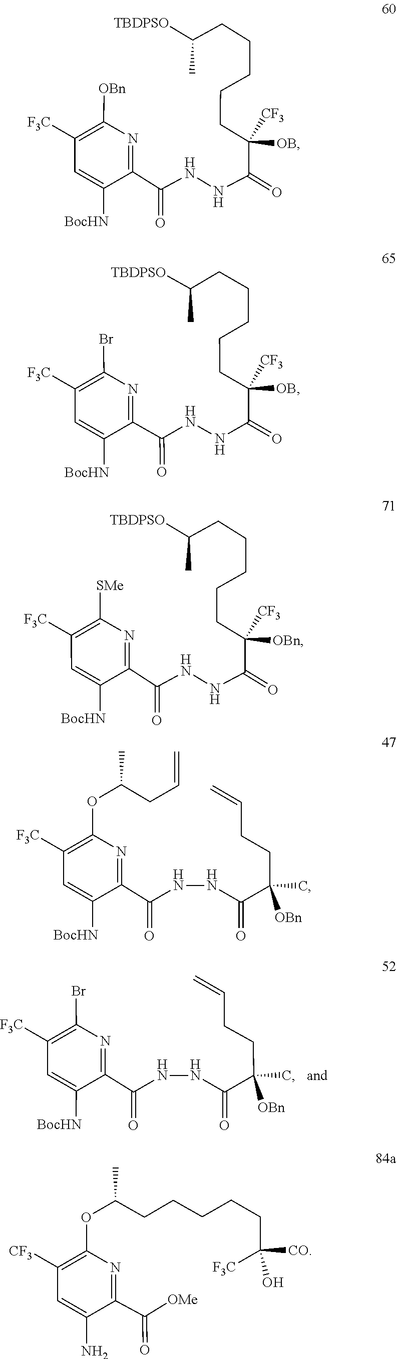

In some embodiments, Compound I can be prepared using a compound selected from:

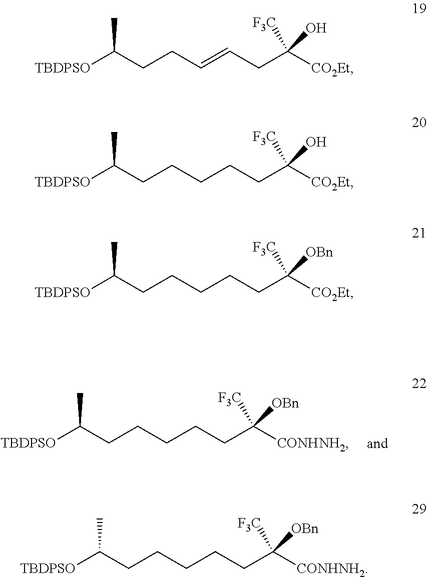

In some embodiments, Compound I can be prepared using a compound selected from:

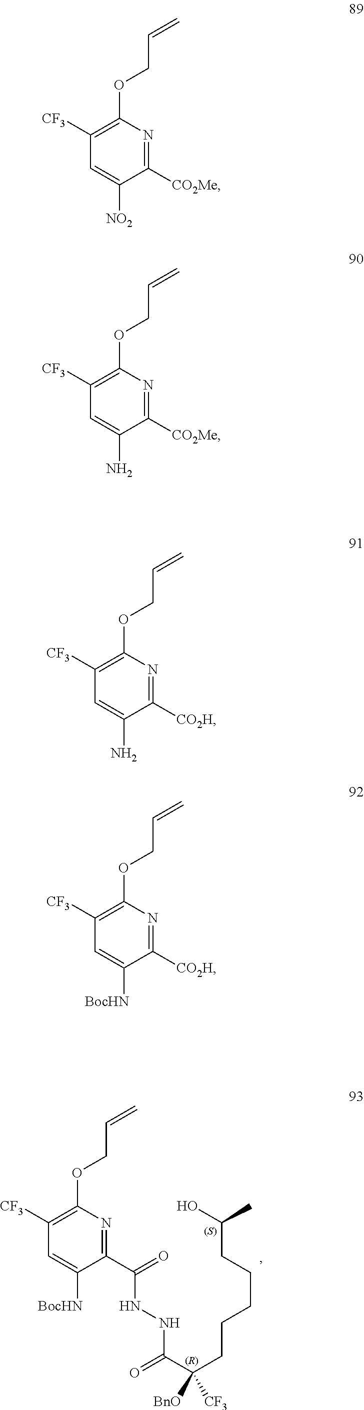

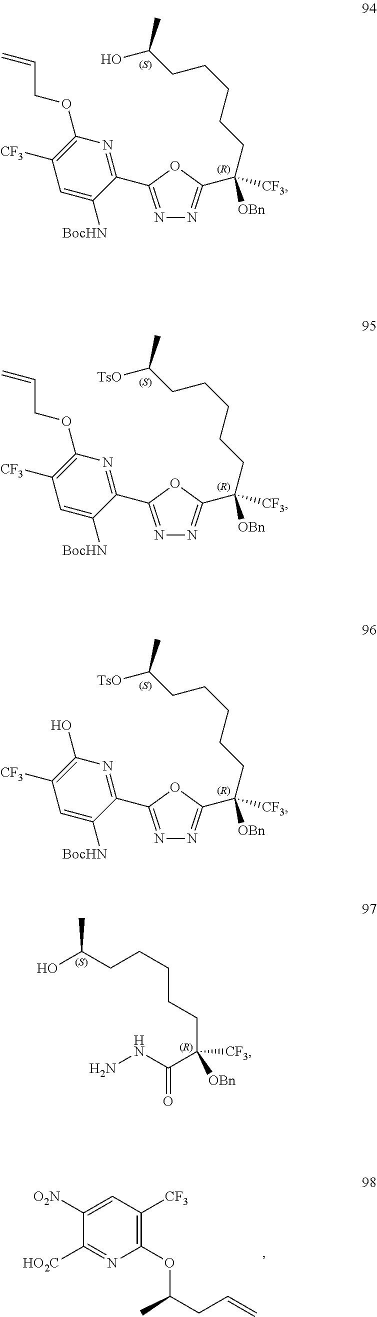

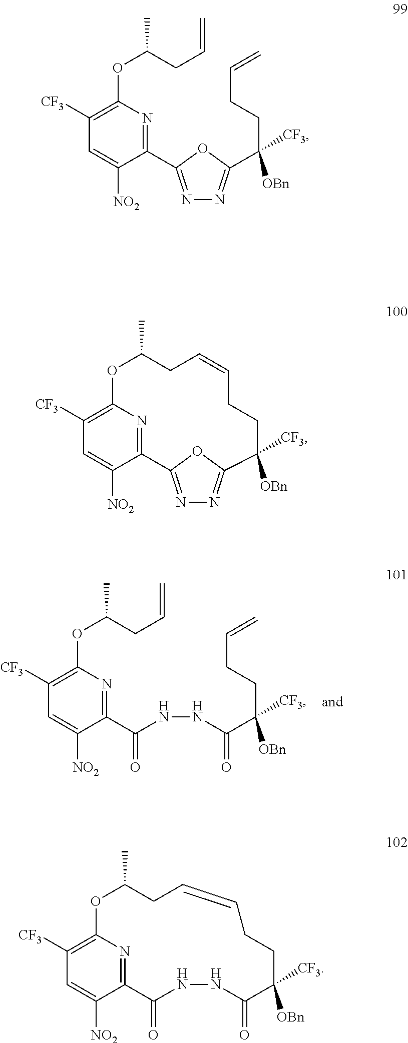 [SMILES: C=CCC[C@@](OCc1ccccc1)(c1nnc(-c2nc(O[C@H](C)CC=C)c(C(F)(F)F)cc2[N+](=O)[O-])o1)C(F)(F)F ; C[C@@H]1C/C=C\CC[C@](OCc2ccccc2)(C(F)(F)F)c2nnc(o2)-c2nc(c(C(F)(F)F)cc2[N+](=O)[O-])O1 ; C=CCC[C@@](OCc1ccccc1)(C(=O)NNC(=O)c1nc(O[C@H](C)CC=C)c(C(F)(F)F)cc1[N+](=O)[O-])C(F)(F)F ; C[C@@H]1C/C=C\CC[C@](OCc2ccccc2)(C(F)(F)F)C(=O)NNC(=O)c2nc(c(C(F)(F)F)cc2[N+](=O)[O-])O1]

In some embodiments, Compound I can be prepared using a compound selected from:

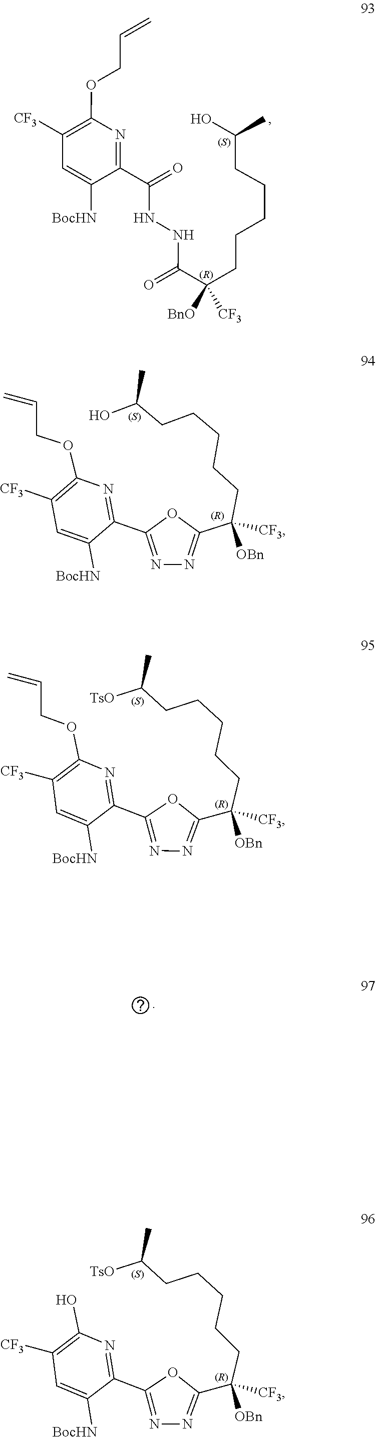

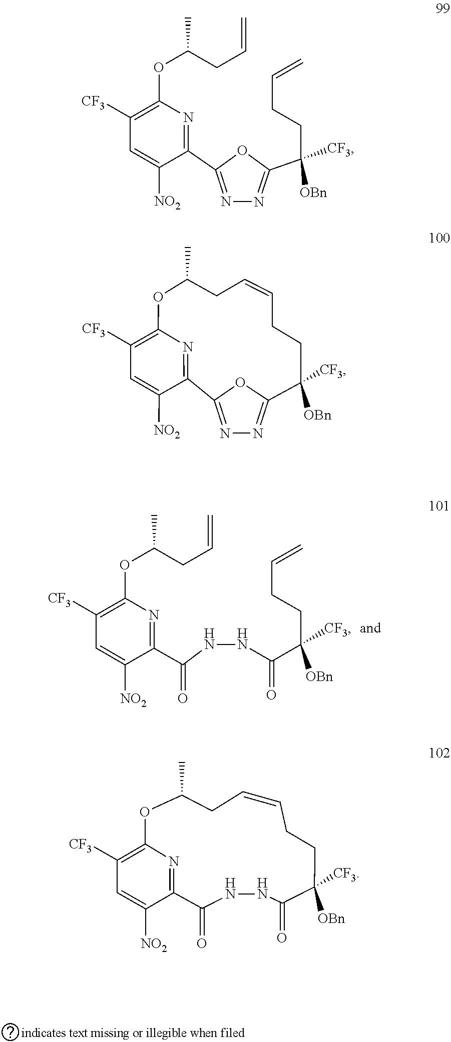 [SMILES: C=CCC[C@@](OCc1ccccc1)(c1nnc(-c2nc(O[C@H](C)CC=C)c(C(F)(F)F)cc2[N+](=O)[O-])o1)C(F)(F)F ; C[C@@H]1C/C=C\CC[C@](OCc2ccccc2)(C(F)(F)F)c2nnc(o2)-c2nc(c(C(F)(F)F)cc2[N+](=O)[O-])O1 ; C=CCC[C@@](OCc1ccccc1)(C(=O)NNC(=O)c1nc(O[C@H](C)CC=C)c(C(F)(F)F)cc1[N+](=O)[O-])C(F)(F)F ; "and" ; C[C@@H]1C/C=C\CC[C@](OCc2ccccc2)(C(F)(F)F)C(=O)NNC(=O)c2nc(c(C(F)(F)F)cc2[N+](=O)[O-])O1]

In some embodiments, a compound of the disclosure is selected from:

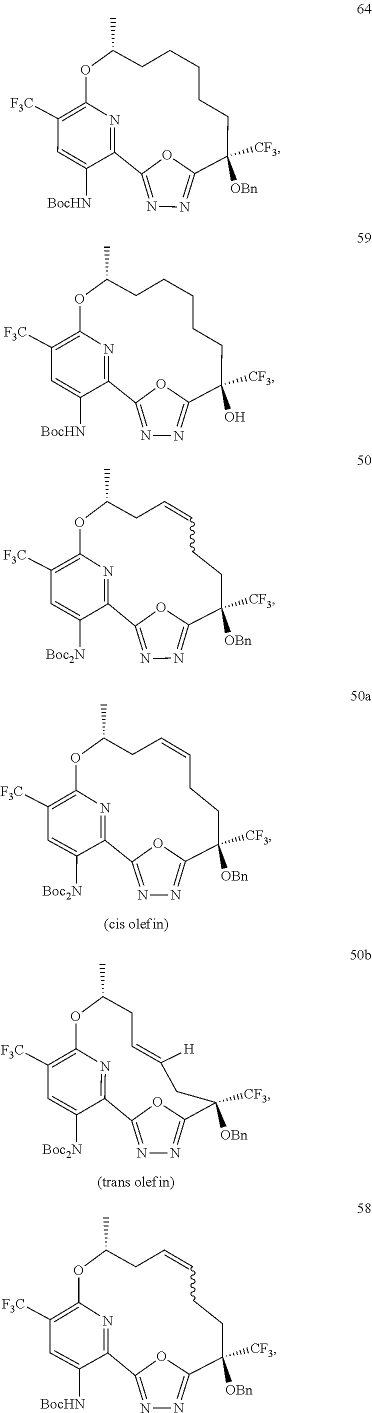

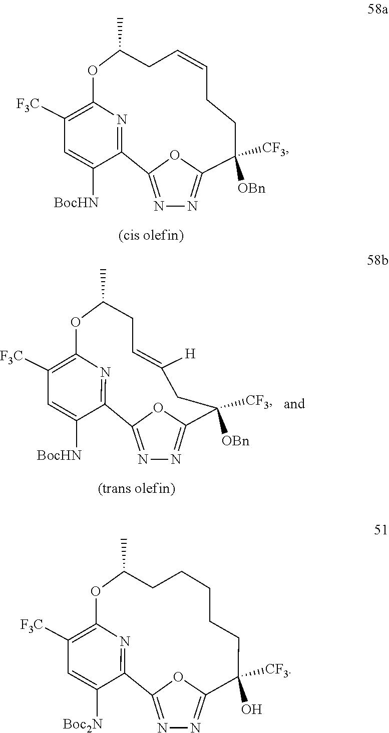

In some embodiments, a compound of the disclosure is selected from:

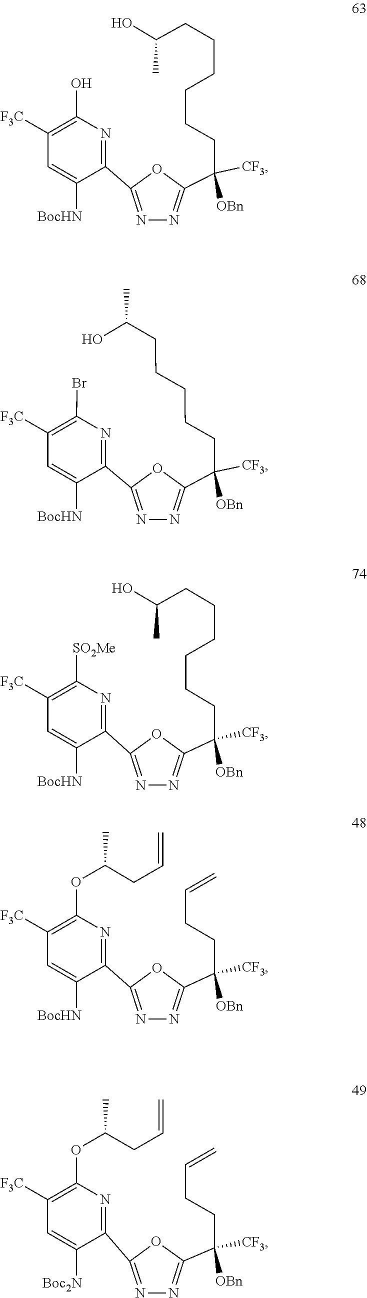

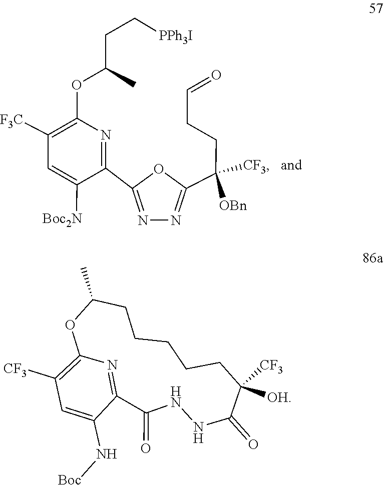

In some embodiments, a compound of the disclosure is selected from:

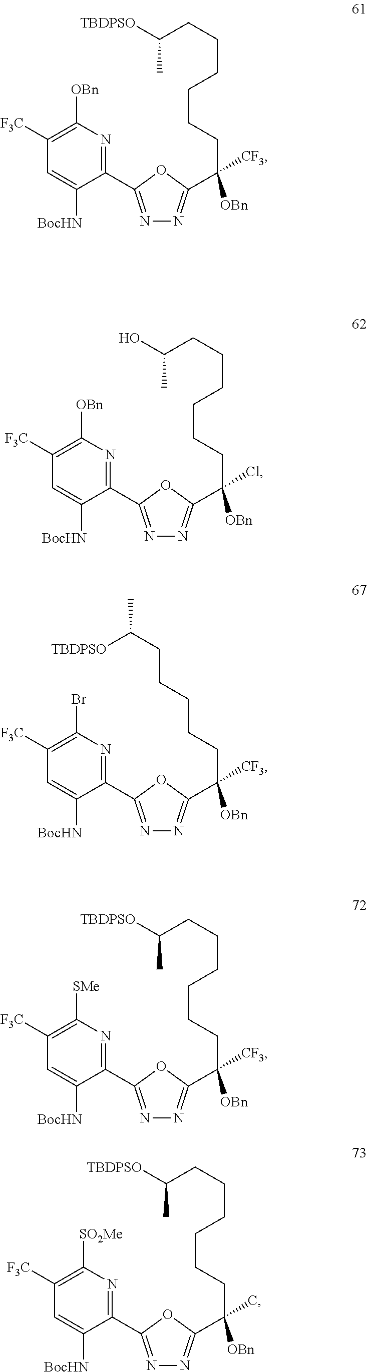

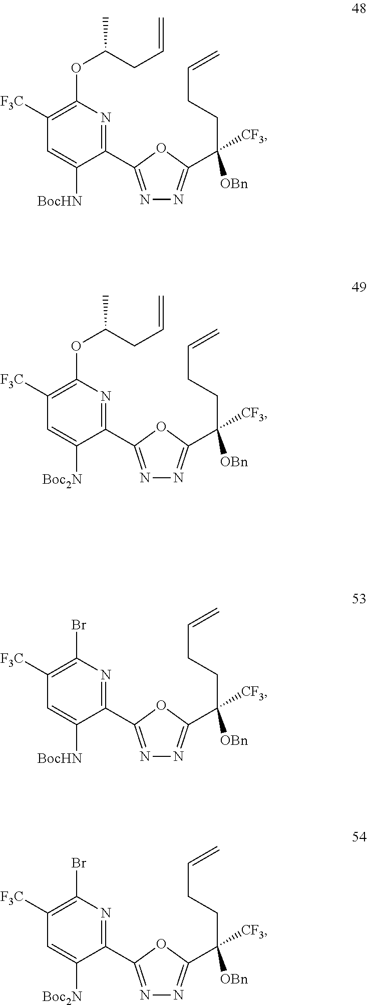

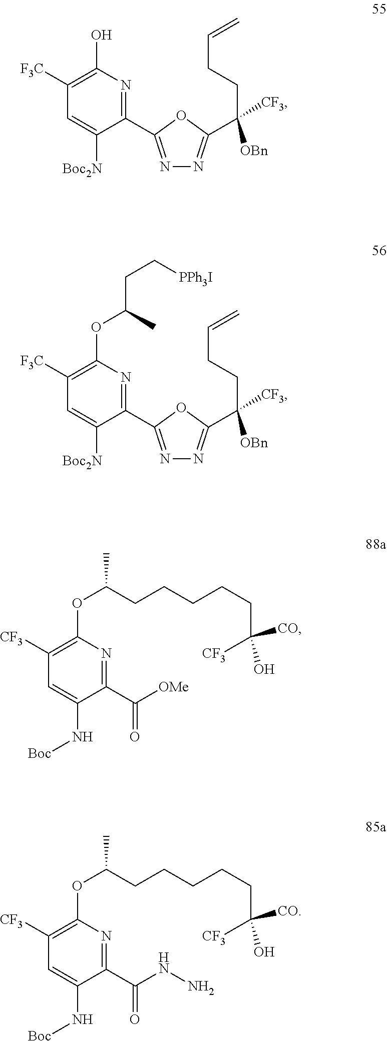

In some embodiments, a compound of the disclosure is selected from:

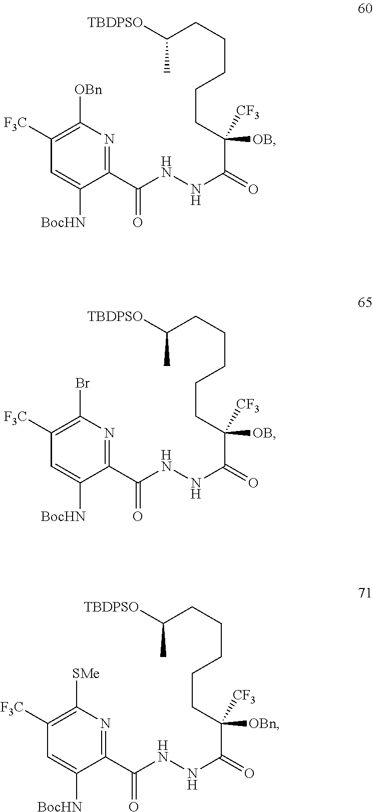

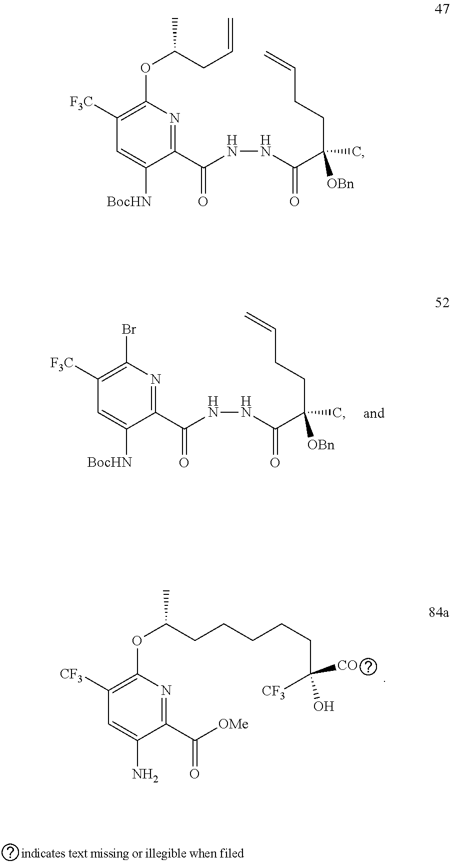

In some embodiments, a compound of the disclosure is selected from:

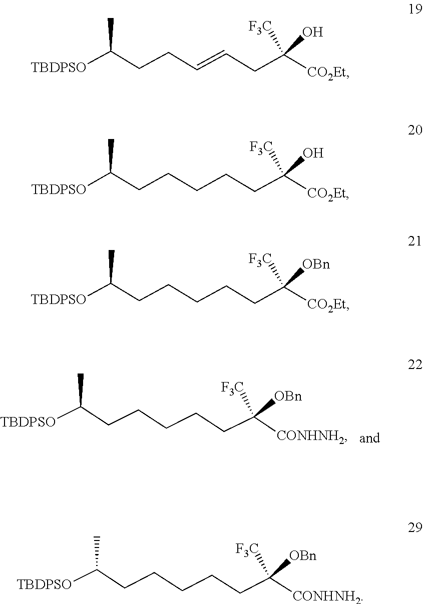

In some embodiments, a compound of the disclosure is selected from:

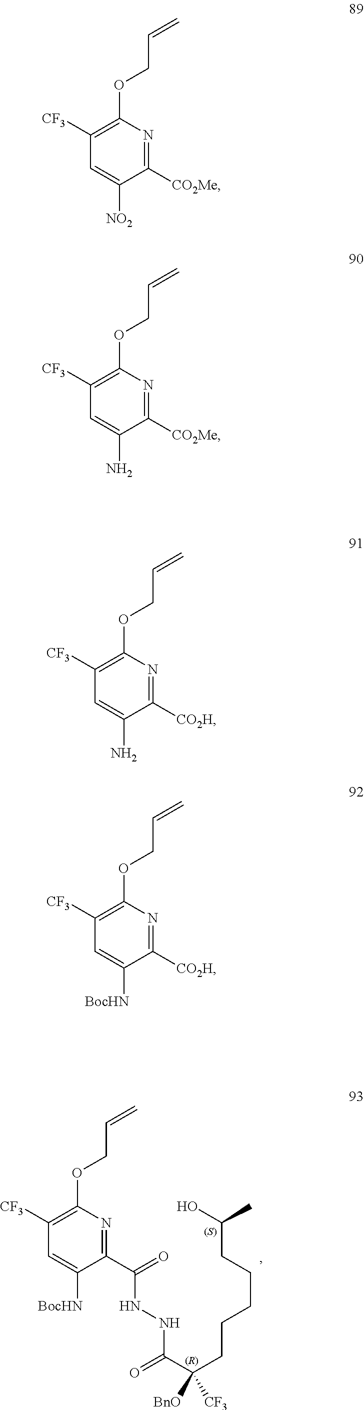

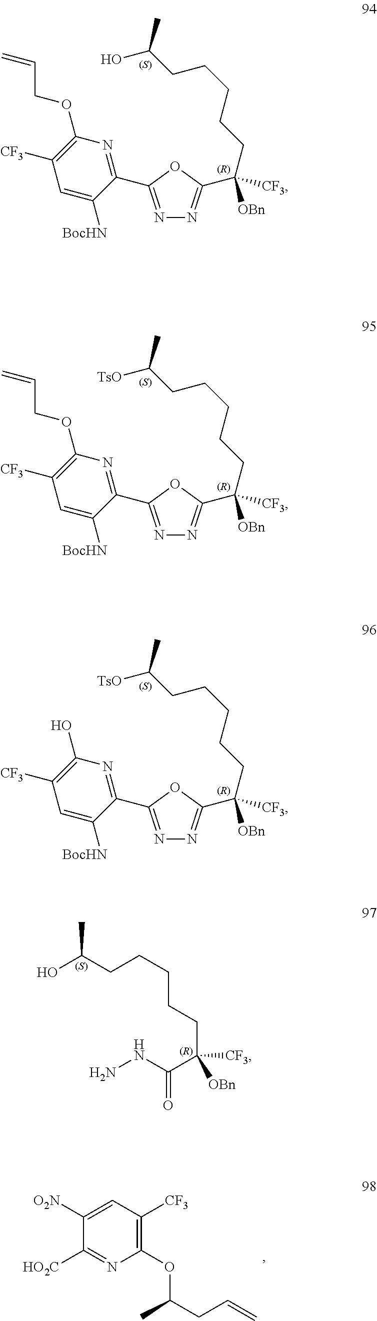

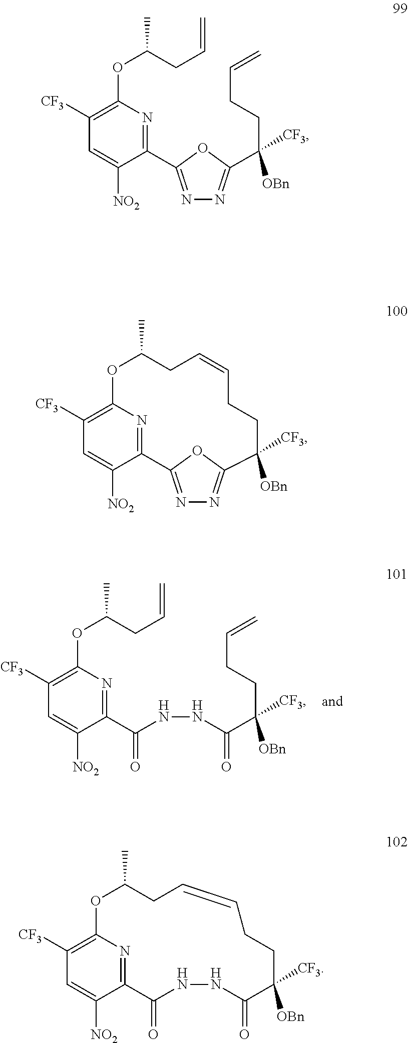 [SMILES: C=CCC[C@@](OCc1ccccc1)(c1nnc(-c2nc(O[C@H](C)CC=C)c(C(F)(F)F)cc2[N+](=O)[O-])o1)C(F)(F)F ; C[C@@H]1C/C=C\CC[C@](OCc2ccccc2)(C(F)(F)F)c2nnc(o2)-c2nc(c(C(F)(F)F)cc2[N+](=O)[O-])O1 ; C=CCC[C@@](OCc1ccccc1)(C(=O)NNC(=O)c1nc(O[C@H](C)CC=C)c(C(F)(F)F)cc1[N+](=O)[O-])C(F)(F)F ; C[C@@H]1C/C=C\CC[C@](OCc2ccccc2)(C(F)(F)F)C(=O)NNC(=O)c2nc(c(C(F)(F)F)cc2[N+](=O)[O-])O1]

In some embodiments, a compound of the disclosure is selected from:

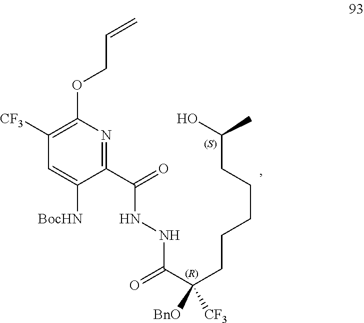

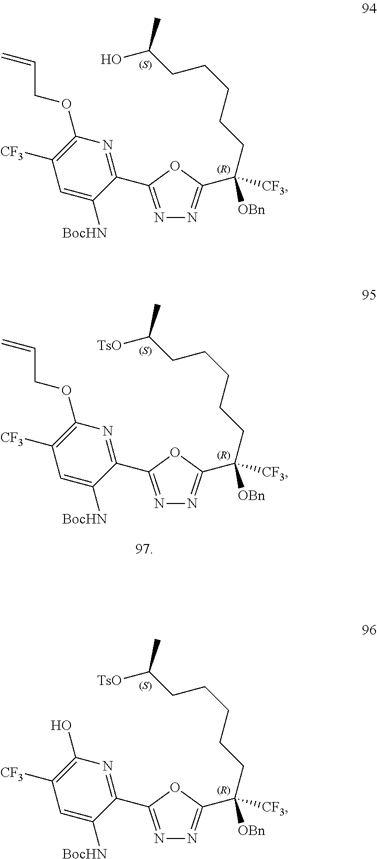

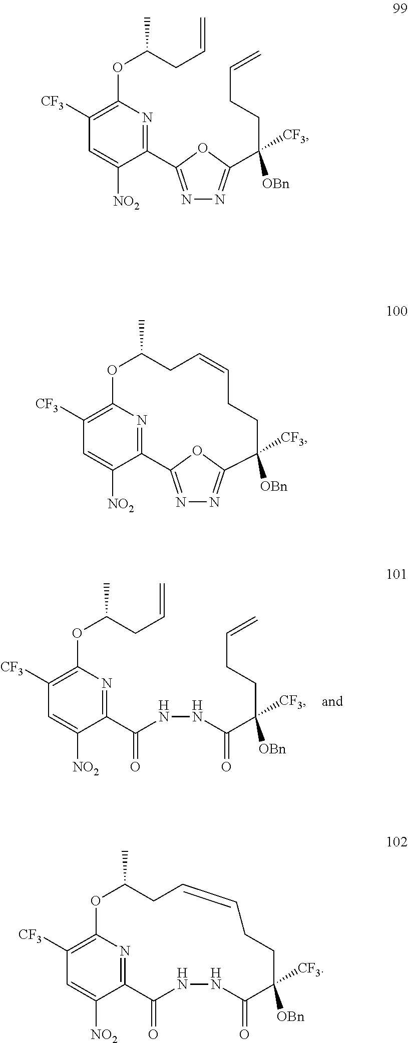 [SMILES: C=CCC[C@@](OCc1ccccc1)(c1nnc(-c2nc(O[C@H](C)CC=C)c(C(F)(F)F)cc2[N+](=O)[O-])o1)C(F)(F)F ; C[C@@H]1C/C=C\CC[C@](OCc2ccccc2)(C(F)(F)F)c2nnc(o2)-c2nc(c(C(F)(F)F)cc2[N+](=O)[O-])O1 ; C=CCC[C@@](OCc1ccccc1)(C(=O)NNC(=O)c1nc(O[C@H](C)CC=C)c(C(F)(F)F)cc1[N+](=O)[O-])C(F)(F)F ; C[C@@H]1C/C=C\CC[C@](OCc2ccccc2)(C(F)(F)F)C(=O)NNC(=O)c2nc(c(C(F)(F)F)cc2[N+](=O)[O-])O1]

### NON-LIMITING EXEMPLARY EMBODIMENTS

### A. Set 1

1. Compound I

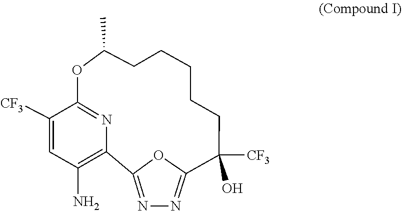 [SMILES: C[C@@H]1CCCCC[C@](O)(C(F)(F)F)C2=NN=C(O2)c2nc(c(C(F)(F)F)cc2N)O1]

as substantially amorphous Compound I neat amorphous form (i.e., wherein less than 15% of Compound I is in crystalline form, wherein less than 10% of Compound I is in crystalline form, wherein less than 5% of Compound I is in crystalline form).
2. The substantially amorphous Compound I neat amorphous form according to Embodiment 1, wherein Compound I is 100% amorphous.
3. The substantially amorphous Compound I neat amorphous form according to Embodiment 1 or Embodiment 2, characterized by an X-ray powder diffractogram substantially similar to FIG. 1.
4. The substantially amorphous Compound I neat amorphous form according to any one of Embodiments 1-3, characterized by a ¹³C SSNMR spectrum having one, two, three, four, five, six, seven, eight, nine, ten, or more peaks selected from 163.8±0.2 ppm, 151.9±0.2 ppm, 137.6±0.2 ppm, 125.8±0.2 ppm, 120.8±0.2 ppm, 117.8±0.2 ppm, 77.3±0.2 ppm, 73.6±0.2 ppm, 34.5±0.2 ppm, 31.4±0.2 ppm, 26.3±0.2 ppm, 22.5±0.2 ppm, and 19.5±0.2 ppm.
5. The substantially amorphous Compound I neat amorphous form according to any one of Embodiments 1-4, characterized by a ¹³C SSNMR spectrum having peaks at 163.8±0.2 ppm, 151.9±0.2 ppm, 137.6±0.2 ppm, 125.8±0.2 ppm, 120.8±0.2 ppm, 117.8±0.2 ppm, 77.3±0.2 ppm, 73.6±0.2 ppm, 34.5±0.2 ppm, 31.4±0.2 ppm, 26.3±0.2 ppm, 22.5±0.2 ppm, and 19.5±0.2 ppm.
6. The substantially amorphous Compound I neat amorphous form according to any one of Embodiments 1-5, characterized by a ¹³C SSNMR spectrum substantially similar to FIG. 4.
7. The substantially amorphous Compound I neat amorphous form according to any one of Embodiments 1-6, characterized by a ¹⁹F SSNMR spectrum having one or two peaks selected from −64.6±0.2 ppm and −77.4±0.2 ppm.
8. The substantially amorphous Compound I neat amorphous form according to any one of Embodiments 1-7, characterized by a ¹⁹F SSNMR spectrum substantially similar to FIG. 5.
9. Substantially crystalline Compound I neat Form A (i.e., wherein less than 15% of Compound I is in amorphous form, wherein less than 10% of Compound I is in amorphous form, wherein less than 5% of Compound I is in amorphous form).
10. The substantially crystalline Compound I neat Form A according to Embodiment 9, wherein Compound I neat Form A is 100% crystalline.
11. The substantially crystalline Compound I neat Form A according to Embodiment 9 or Embodiment 10, characterized by an X-ray powder diffractogram having one or two signals selected from 4.6±0.2 degrees two-theta and 20.8±0.2 degrees two-theta.
12. The substantially crystalline Compound I neat Form A according to any one of Embodiments 9-11, characterized by an X-ray powder diffractogram having (a) one or two signals selected from 4.6±0.2 degrees two-theta and 20.8±0.2 degrees two-theta, and (b) one or two signals selected from 9.2±0.2 degrees two-theta, and 18.4±0.2 degrees two-theta.
13. The substantially crystalline Compound I neat Form A according to any one of Embodiments 9-12, characterized by an X-ray powder diffractogram having two, three, or four signals selected from 4.6±0.2 degrees two-theta, 9.2±0.2 degrees two-theta, 18.4±0.2 degrees two-theta, and 20.8±0.2 degrees two-theta.
14. The substantially crystalline Compound I neat Form A according to any one of Embodiments 9-13, characterized by an X-ray powder diffractogram substantially similar to FIG. 6.
15. Substantially crystalline Compound I neat Form B (i.e., wherein less than 15% of Compound I is in amorphous form, wherein less than 10% of Compound I is in amorphous form, wherein less than 5% of Compound I is in amorphous form).
16. The substantially crystalline Compound I neat Form B according to Embodiment 15, wherein Compound I neat Form B is 100% crystalline.
17. The substantially crystalline Compound I neat Form B according to Embodiment 15 or Embodiment 16, characterized by an X-ray powder diffractogram having one, two, three, four, five, or six signals selected from 5.7±0.2 degrees two-theta, 6.1±0.2 degrees two-theta, 7.6±0.2 degrees two-theta, 10.3±0.2 degrees two-theta, 10.6±0.2 degrees two-theta, and 12.3±0.2 degrees two-theta.
18. The substantially crystalline Compound I neat Form B according to any one of Embodiments 15-17, characterized by an X-ray powder diffractogram having (a) one, two, three, four, five, or six signals selected from 5.7±0.2 degrees two-theta, 6.1±0.2 degrees two-theta, 7.6±0.2 degrees two-theta, 10.3±0.2 degrees two-theta, 10.6±0.2 degrees two-theta, and 12.3±0.2 degrees two-theta, and (b) one or two signals selected from 9.3±0.2 degrees two-theta, and 16.1±0.2 degrees two-theta.
19. The substantially crystalline Compound I neat Form B according to any one of Embodiments 15-18, characterized by an X-ray powder diffractogram having two, three, four, five, six, seven, or eight signals selected from 5.7±0.2 degrees two-theta, 6.1±0.2 degrees two-theta, 7.6±0.2 degrees two-theta, 9.3±0.2 degrees two-theta, 10.3±0.2 degrees two-theta, 10.6±0.2 degrees two-theta, 12.3±0.2 degrees two-theta, and 16.1±0.2 degrees two-theta.
20. The substantially crystalline Compound I neat Form B according to any one of Embodiments 15-19, characterized by an X-ray powder diffractogram having signals at 5.7±0.2 degrees two-theta, 6.1±0.2 degrees two-theta, 7.6±0.2 degrees two-theta, 9.3±0.2 degrees two-theta, 10.3±0.2 degrees two-theta, 10.6±0.2 degrees two-theta, 12.3±0.2 degrees two-theta, and 16.1±0.2 degrees two-theta.
21. The substantially crystalline Compound I neat Form B according to any one of Embodiments 15-20, characterized by an X-ray powder diffractogram substantially similar to FIG. 9.
22. The substantially crystalline Compound I neat Form B according to any one of Embodiments 15-21, characterized by a ¹³C SSNMR spectrum having one, two, three, four, five, six, seven, eight, nine, ten, or more peaks selected from 165.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm.
23. The substantially crystalline Compound I neat Form B according to any one of Embodiments 15-22, characterized by a ¹³C SSNMR spectrum having peaks at 165.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm.
24. The substantially crystalline Compound I neat Form B according to any one of Embodiments 15-23, characterized by a ¹³C SSNMR spectrum having (a) one, two, three, four, five, six, seven, eight, nine, ten, or more peaks selected from 165.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm, and (b) one, two, or three peaks selected from 164.7±0.2 ppm, 163.8±0.2 ppm, and 74.4±0.2 ppm.
25. The substantially crystalline Compound I neat Form B according to any one of Embodiments 15-24, characterized by a ¹³C SSNMR spectrum having four, five, six, seven, eight, nine, ten, or more peaks selected from 165.8±0.2 ppm, 164.7±0.2 ppm, 163.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 74.4±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm.
26. The substantially crystalline Compound I neat Form B according to any one of Embodiments 15-25, characterized by a ¹³C SSNMR spectrum having peaks at 165.8±0.2 ppm, 164.7±0.2 ppm, 163.8±0.2 ppm, 154.2±0.2 ppm, 151.8±0.2 ppm, 140.1±0.2 ppm, 138.1±0.2 ppm, 136.2±0.2 ppm, 134.9±0.2 ppm, 131.7±0.2 ppm, 129.4±0.2 ppm, 125.5±0.2 ppm, 123.0±0.2 ppm, 120.2±0.2 ppm, 117.5±0.2 ppm, 78.3±0.2 ppm, 74.4±0.2 ppm, 73.6±0.2 ppm, 37.6±0.2 ppm, 34.0±0.2 ppm, 29.9±0.2 ppm, 27.3±0.2 ppm, 22.7±0.2 ppm, 21.1±0.2 ppm, and 18.9±0.2 ppm.
27. The substantially crystalline Compound I neat Form B according to any one of Embodiments 15-26, characterized by a ¹³C SSNMR spectrum substantially similar to FIG. 12.
28. The substantially crystalline Compound I neat Form B according to any one of Embodiments 15-27, characterized by a ¹⁹F SSNMR spectrum having one, two, or three peaks selected from −64.3±0.2 ppm, −65.9±0.2 ppm, and −76.5±0.2 ppm.
29. The substantially crystalline Compound I neat Form B according to any one of Embodiments 15-28, characterized by a ¹⁹F SSNMR spectrum substantially similar to FIG. 13.
30. Substantially crystalline Compound I hemihydrate Form C (i.e., wherein less than 15% of Compound I is in amorphous form, wherein less than 10% of Compound I is in amorphous form, wherein less than 5% of Compound I is in amorphous form).
31. The substantially crystalline Compound I hemihydrate Form C according to Embodiment 30, wherein Compound I hemihydrate Form C is 100% crystalline.
32. The substantially crystalline Compound I hemihydrate Form C according to Embodiment 30 or Embodiment 31, characterized by an X-ray powder diffractogram having one, two, three, or four signals selected from 4.8±0.2 degrees two-theta, 16.4±0.2 degrees two-theta, 19.3±0.2 degrees two-theta, and 21.1±0.2 degrees two-theta
33. The substantially crystalline Compound I hemihydrate Form C according to any one of Embodiments 30-32, characterized by an X-ray powder diffractogram having one, two, three, four, or five signals selected from 13.1±0.2 degrees two-theta, 16.4±0.2 degrees two-theta, 18.4±0.2 degrees two-theta, 19.6±0.2 degrees two-theta, and 21.1±0.2 degrees two-theta.
34. The substantially crystalline Compound I hemihydrate Form C according to any one of Embodiments 30-33, characterized by an X-ray powder diffractogram having one, two, three, four, five, six, seven, eight, nine, or ten signals selected from 4.8±0.2 degrees two-theta, 13.1±0.2 degrees two-theta, 16.4±0.2 degrees two-theta, 18.4±0.2 degrees two-theta, 19.3±0.2 degrees two-theta, 19.6±0.2 degrees two-theta, 21.1±0.2 degrees two-theta, 24.0±0.2 degrees two-theta, 24.6±0.2 degrees two-theta, and 27.1±0.2 degrees two-theta.
35. The substantially crystalline Compound I hemihydrate Form C according to any one of Embodiments 30-34, characterized by an X-ray powder diffractogram having one, two, three, four, five, six, seven, eight, nine, ten, or more signals selected from 4.8±0.2 degrees two-theta, 8.2±0.2 degrees two-theta, 9.3±0.2 degrees two-theta, 11.2±0.2 degrees two-theta, 12.5±0.2 degrees two-theta, 13.1±0.2 degrees two-theta, 16.4±0.2 degrees two-theta, 18.4±0.2 degrees two-theta, 19.3±0.2 degrees two-theta, 19.6±0.2 degrees two-theta, 21.1±0.2 degrees two-theta, 21.6±0.2 degrees two-theta, 22.8±0.2 degrees two-theta, 23.5±0.2 degrees two-theta, 24±0.2 degrees two-theta, 24.6±0.2 degrees two-theta, 25.8±0.2 degrees two-theta, 27.1±0.2 degrees two-theta, 29.6±0.2 degrees two-theta, and 33.4±0.2 degrees two-theta.
36. The substantially crystalline Compound I hemihydrate Form C according to any one of Embodiments 30-35, characterized by an X-ray powder diffractogram having signals at 4.8±0.2 degrees two-theta, 8.2±0.2 degrees two-theta, 9.3±0.2 degrees two-theta, 11.2±0.2 degrees two-theta, 12.5±0.2 degrees two-theta, 13.1±0.2 degrees two-theta, 16.4±0.2 degrees two-theta, 18.4±0.2 degrees two-theta, 19.3±0.2 degrees two-theta, 19.6±0.2 degrees two-theta, 21.1±0.2 degrees two-theta, 21.6±0.2 degrees two-theta, 22.8±0.2 degrees two-theta, 23.5±0.2 degrees two-theta, 24±0.2 degrees two-theta, 24.6±0.2 degrees two-theta, 25.8±0.2 degrees two-theta, 27.1±0.2 degrees two-theta, 29.6±0.2 degrees two-theta, and 33.4±0.2 degrees two-theta.
37. The substantially crystalline Compound I hemihydrate Form C according to any one of Embodiments 30-36, characterized by an X-ray powder diffractogram substantially similar to FIG. 14.
38. The substantially crystalline Compound I hemihydrate Form C according to any one of Embodiments 30-37, characterized by a ¹³C SSNMR spectrum having one, two, three, four, five, six, seven, eight, nine, ten, or more peaks selected from 163.8±0.2 ppm, 151.3±0.2 ppm, 139.1±0.2 ppm, 137.7±0.2 ppm, 127.2±0.2 ppm, 125.8±0.2 ppm, 119.9±0.2 ppm, 118.4±0.2 ppm, 75.6±0.2 ppm, 73.6±0.2 ppm, 35.8±0.2 ppm, 32.2±0.2 ppm, 29.6±0.2 ppm, 24.6±0.2 ppm, 22.1±0.2 ppm, and 19.2±0.2 ppm.
39. The substantially crystalline Compound I hemihydrate Form C according to any one of Embodiments 30-38, characterized by a ¹³C SSNMR spectrum having peaks at 163.8±0.2 ppm, 151.3±0.2 ppm, 139.1±0.2 ppm, 137.7±0.2 ppm, 127.2±0.2 ppm, 125.8±0.2 ppm, 119.9±0.2 ppm, 118.4±0.2 ppm, 75.6±0.2 ppm, 73.6±0.2 ppm, 35.8±0.2 ppm, 32.2±0.2 ppm, 29.6±0.2 ppm, 24.6±0.2 ppm, 22.1±0.2 ppm, and 19.2±0.2 ppm.
40. The substantially crystalline Compound I hemihydrate Form C according to any one of Embodiments 30-39, characterized by a ¹³C SSNMR spectrum substantially similar to FIG. 17.
41. The substantially crystalline Compound I hemihydrate Form C according to any one of Embodiments 30-40, characterized as having a ¹⁹F SSNMR spectrum with one or two peaks selected from −65.5±0.2 ppm and −77.4±0.2 ppm.
42. The substantially crystalline Compound I hemihydrate Form C according to any one of Embodiments 30-41, characterized by a ¹⁹F SSNMR spectrum substantially similar to FIG. 18.
43. The substantially crystalline Compound I hemihydrate Form C according to any one of Embodiments 30-42, characterized by a monoclinic crystal system, P 2₁ space group, and unit cell dimensions measured at 100 K on a Bruker diffractometer utilizing Cu K_{α} radiation (λ=1.54178 Å) of

| | | | |
|---|---|---|---|
| a | 12.1 ± 0.1 Å | α | 90° |
| b | 8.6 ± 0.1 Å | β | 98.2 ± 0.1° |
| c | 18.9 ± 0.1 Å | γ | 90°. |

44. Substantially crystalline Compound I neat Form D (i.e., wherein less than 15% of Compound I is in amorphous form, wherein less than 10% of Compound I is in amorphous form, wherein less than 5% of Compound I is in amorphous form).
45. The substantially crystalline Compound I neat Form D according to Embodiment 44, wherein Compound I neat Form D is 100% crystalline.
46. The substantially crystalline Compound I neat Form D according to Embodiment 44 or Embodiment 45, characterized by an X-ray powder diffractogram having two or three signals selected from 8.4±0.2 degrees two-theta, 14.3±0.2 degrees two-theta, and 20.3±0.2 degrees two-theta.
47. The substantially crystalline Compound I neat Form D according to any one of Embodiments 44-46, characterized by an X-ray powder diffractogram having one, two, three, four, five, six, seven, eight, nine, ten, or more signals selected from 8.4±0.2 degrees two-theta, 8.8±0.2 degrees two-theta, 14.3±0.2 degrees two-theta, 14.8±0.2 degrees two-theta, 15.4±0.2 degrees two-theta, 16.77±0.2 degrees two-theta, 16.85±0.2 degrees two-theta, 19.6±0.2 degrees two-theta, 20.0±0.2 degrees two-theta, 20.3±0.2 degrees two-theta, 21.7±0.2 degrees two-theta, 22.5±0.2 degrees two-theta, 24.7±0.2 degrees two-theta, 25.2±0.2 degrees two-theta, 26.2±0.2 degrees two-theta, 26.45±0.2 degrees two-theta, 26.52±0.2 degrees two-theta, 27.8±0.2 degrees two-theta, and 28.8±0.2 degrees two-theta.
48. The substantially crystalline Compound I neat Form D according to any one of Embodiments 44-47, characterized by an X-ray powder diffractogram having (a) one, two, three, four, five, six, seven, eight, nine, ten, or more signals selected from 8.4±0.2 degrees two-theta, 8.8±0.2 degrees two-theta, 14.3±0.2 degrees two-theta, 14.8±0.2 degrees two-theta, 15.4±0.2 degrees two-theta, 16.77±0.2 degrees two-theta, 16.85±0.2 degrees two-theta, 19.6±0.2 degrees two-theta, 20.0±0.2 degrees two-theta, 20.3±0.2 degrees two-theta, 21.7±0.2 degrees two-theta, 22.5±0.2 degrees two-theta, 24.7±0.2 degrees two-theta, 25.2±0.2 degrees two-theta, 26.2±0.2 degrees two-theta, 26.45±0.2 degrees two-theta, 26.52±0.2 degrees two-theta, 27.8±0.2 degrees two-theta, and 28.8±0.2 degrees two-theta, and (b) one, two, or three signals selected from 16.0±0.2 degrees two-theta, 18.55±0.2 degrees two-theta, and 18.64±0.2 degrees two-theta.
49. The substantially crystalline Compound I neat Form D according to any one of Embodiments 44-48, characterized by an X-ray powder diffractogram having three, four, five, six, seven, eight, nine, ten, or more signals selected from 8.4±0.2 degrees two-theta, 8.8±0.2 degrees two-theta, 14.3±0.2 degrees two-theta, 14.8±0.2 degrees two-theta, 15.4±0.2 degrees two-theta, 16.0±0.2 degrees two-theta, 16.77±0.2 degrees two-theta, 16.85±0.2 degrees two-theta, 18.55±0.2 degrees two-theta, 18.64±0.2 degrees two-theta, 19.6±0.2 degrees two-theta, 20.0±0.2 degrees two-theta, 20.3±0.2 degrees two-theta, 21.7±0.2 degrees two-theta, 22.5±0.2 degrees two-theta, 24.7±0.2 degrees two-theta, 25.2±0.2 degrees two-theta, 26.2±0.2 degrees two-theta, 26.45±0.2 degrees two-theta, 26.52±0.2 degrees two-theta, 27.8±0.2 degrees two-theta, and 28.8±0.2 degrees two-theta.
50. The substantially crystalline Compound I neat Form D according to any one of Embodiments 44-49, characterized by an X-ray powder diffractogram having signals at 8.4±0.2 degrees two-theta, 8.8±0.2 degrees two-theta, 14.3±0.2 degrees two-theta, 14.8±0.2 degrees two-theta, 15.4±0.2 degrees two-theta, 16.0±0.2 degrees two-theta, 16.77±0.2 degrees two-theta, 16.85±0.2 degrees two-theta, 18.55±0.2 degrees two-theta, 18.64±0.2 degrees two-theta, 19.6±0.2 degrees two-theta, 20.0±0.2 degrees two-theta, 20.3±0.2 degrees two-theta, 21.7±0.2 degrees two-theta, 22.5±0.2 degrees two-theta, 24.7±0.2 degrees two-theta, 25.2±0.2 degrees two-theta, 26.2±0.2 degrees two-theta, 26.45±0.2 degrees two-theta, 26.52±0.2 degrees two-theta, 27.8±0.2 degrees two-theta, and 28.8±0.2 degrees two-theta.
51. The substantially crystalline Compound I neat Form D according to any one of Embodiments 44-50, characterized by an X-ray powder diffractogram substantially similar to FIG. 19.
52. The substantially crystalline Compound I neat Form D according to any one of Embodiments 44-51, characterized as having a ¹³C SSNMR spectrum with one, two, three, four, five, six, seven, eight, or nine peaks selected from 152.2±0.2 ppm, 137.7±0.2 ppm, 127.3±0.2 ppm, 120.8±0.2 ppm, 118.1±0.2 ppm, 75.7±0.2 ppm, 35.9±0.2 ppm, 30.4±0.2 ppm, 22.1±0.2 ppm, and 17.7±0.2 ppm.
53. The substantially crystalline Compound I neat Form D according to any one of Embodiments 44-52, characterized as having a ¹³C SSNMR spectrum with (a) one, two, three, four, five, six, seven, eight, nine, or ten peaks selected from 152.2±0.2 ppm, 137.7±0.2 ppm, 127.3±0.2 ppm, 120.8±0.2 ppm, 118.1±0.2 ppm, 75.7±0.2 ppm, 35.9±0.2 ppm, 30.4±0.2 ppm, 22.1±0.2 ppm, and 17.7±0.2 ppm, and (b) one, two, or three peaks selected from 164.6±0.2 ppm, 163.8±0.2 ppm, and 74.2±0.2 ppm.
54. The substantially crystalline Compound I neat Form D according to any one of Embodiments 44-53, characterized as having a ¹³C SSNMR spectrum with four, five, six, seven, eight, nine, ten, or more peaks selected from 164.6±0.2 ppm, 163.8±0.2 ppm, 152.2±0.2 ppm, 137.7±0.2 ppm, 127.3±0.2 ppm, 120.8±0.2 ppm, 118.1±0.2 ppm, 75.7±0.2 ppm, 74.2±0.2 ppm, 35.9±0.2 ppm, 30.4±0.2 ppm, 22.1±0.2 ppm, and 17.7±0.2 ppm.
55. The substantially crystalline Compound I neat Form D according to any one of Embodiments 44-54, characterized as having a ¹³C SSNMR spectrum with peaks at 164.6±0.2 ppm, 163.8±0.2 ppm, 152.2±0.2 ppm, 137.7±0.2 ppm, 127.3±0.2 ppm, 120.8±0.2 ppm, 118.1±0.2 ppm, 75.7±0.2 ppm, 74.2±0.2 ppm, 35.9±0.2 ppm, 30.4±0.2 ppm, 22.1±0.2 ppm, and 17.7±0.2 ppm.
56. The substantially crystalline Compound I neat Form D according to any one of Embodiments 44-55, characterized by a ¹³C SSNMR spectrum substantially similar to FIG. 23.
57. The substantially crystalline Compound I neat Form D according to any one of Embodiments 44-56, characterized as having a ¹⁹F SSNMR spectrum with one or two peaks selected from −62.4±0.2 ppm and −77.2±0.2 ppm.
58. The substantially crystalline Compound I neat Form D according to any one of Embodiments 44-57, characterized by a ¹⁹F SSNMR spectrum substantially similar to FIG. 24.
59. The substantially crystalline Compound I neat Form D according to any one of Embodiments 44-58, characterized by an monoclinic crystal system, P 2₁ space group, and unit cell dimensions measured at 250 K on a Bruker diffractometer utilizing Cu K_{α} radiation (λ=1.54178 Å) of

| | | | |
|---|---|---|---|
| a | 7.9 ± 0.1 Å | α | 90° |
| b | 11.5 ± 0.1 Å | β | 90.02 ± 0.10° |
| c | 21.0 ± 0.2 Å | γ | 90°. |

60. Substantially crystalline Compound I neat Form E (i.e., wherein less than 15% of Compound I is in amorphous form, wherein less than 10% of Compound I is in amorphous form, wherein less than 5% of Compound I is in amorphous form).
61. The substantially crystalline Compound I neat Form E according to Embodiment 60, wherein Compound I neat Form E is 100% crystalline.
62. The substantially crystalline Compound I neat Form E according to any one of Embodiments 60-61, characterized by an orthorhombic crystal system, P 2₁2₁2₁ space group, and unit cell dimensions measured at 100 K on a Bruker diffractometer utilizing Cu Kₐ radiation (λ=1.54178 Å) of

| | | | |
|---|---|---|---|
| a | 8.3 ± 0.1 Å | α | 90° |
| b | 11.2 ± 0.1 Å | β | 90° |
| c | 20.2 ± 0.1 Å | γ | 90°. |

63. Substantially crystalline Compound I acetic acid solvate (i.e., wherein less than 15% of Compound I is in amorphous form, wherein less than 10% of Compound I is in amorphous form, wherein less than 5% of Compound I is in amorphous form).
64. The substantially crystalline Compound I acetic acid solvate according to Embodiment 63, wherein Compound I acetic acid solvate is 100% crystalline.
65. The substantially crystalline Compound I acetic acid solvate according to Embodiment 63 or Embodiment 64, characterized by an X-ray powder diffractogram having one, two, three, four, or five signals selected from 5.4±0.2 degrees two-theta, 10.2±0.2 degrees two-theta, 14.2±0.2 degrees two-theta, 15.9±0.2 degrees two-theta, and 20.2±0.2 degrees two-theta.
66. The substantially crystalline Compound I acetic acid solvate according to any one of Embodiments 63-65, characterized by an X-ray powder diffractogram having one, two, three, four, five, six, seven, eight, nine, or ten signals selected from 5.4±0.2 degrees two-theta, 10.2±0.2 degrees two-theta, 14.2±0.2 degrees two-theta, 15.9±0.2 degrees two-theta, 18.5±0.2 degrees two-theta, 19.5±0.2 degrees two-theta, 19.8±0.2 degrees two-theta, 20.2±0.2 degrees two-theta, 20.6±0.2 degrees two-theta, and 21.6±0.2 degrees two-theta.
67. The substantially crystalline Compound I acetic acid solvate according to any one of Embodiments 63-66, characterized by an X-ray powder diffractogram having one, two, three, four, five, six, seven, eight, nine, ten, or more signals selected from 5.4±0.2 degrees two-theta, 8.3±0.2 degrees two-theta, 10.2±0.2 degrees two-theta, 10.4±0.2 degrees two-theta, 10.9±0.2 degrees two-theta, 11.7±0.2 degrees two-theta, 13.1±0.2 degrees two-theta, 13.2±0.2 degrees two-theta, 13.8±0.2 degrees two-theta, 14.2±0.2 degrees two-theta, 15.9±0.2 degrees two-theta, 16.7±0.2 degrees two-theta, 17.5±0.2 degrees two-theta, 18.0±0.2 degrees two-theta, 18.5±0.2 degrees two-theta, 18.9±0.2 degrees two-theta, 19.5±0.2 degrees two-theta, 19.8±0.2 degrees two-theta, 20.2±0.2 degrees two-theta, 20.6±0.2 degrees two-theta, 21.6±0.2 degrees two-theta, 22.5±0.2 degrees two-theta, 25.0±0.2 degrees two-theta, 25.3±0.2 degrees two-theta, and 26.3±0.2 degrees two-theta.
68. The substantially crystalline Compound I acetic acid solvate according to any one of Embodiments 63-67, characterized by an X-ray powder diffractogram having signals at 5.4±0.2 degrees two-theta, 8.3±0.2 degrees two-theta, 10.2±0.2 degrees two-theta, 10.4±0.2 degrees two-theta, 10.9±0.2 degrees two-theta, 11.7±0.2 degrees two-theta, 13.1±0.2 degrees two-theta, 13.2±0.2 degrees two-theta, 13.8±0.2 degrees two-theta, 14.2±0.2 degrees two-theta, 15.9±0.2 degrees two-theta, 16.7±0.2 degrees two-theta, 17.5±0.2 degrees two-theta, 18.0±0.2 degrees two-theta, 18.5±0.2 degrees two-theta, 18.9±0.2 degrees two-theta, 19.5±0.2 degrees two-theta, 19.8±0.2 degrees two-theta, 20.2±0.2 degrees two-theta, 20.6±0.2 degrees two-theta, 21.6±0.2 degrees two-theta, 22.5±0.2 degrees two-theta, 25.0±0.2 degrees two-theta, 25.3±0.2 degrees two-theta, and 26.3±0.2 degrees two-theta.
69. The substantially crystalline Compound I acetic acid solvate according to any one of Embodiments 63-68, characterized by an X-ray powder diffractogram substantially similar to FIG. 25.
70. Substantially Compound I heptane solvate Form B (i.e., wherein less than 15% of Compound I is in amorphous form, wherein less than 10% of Compound I is in amorphous form, wherein less than 5% of Compound I is in amorphous form).
71. The substantially crystalline Compound I heptane solvate Form B according to Embodiment 70, wherein Compound I heptane solvate Form B is 100% crystalline.
72. The substantially crystalline Compound I heptane solvate Form B according to Embodiment 70 or Embodiment 71, characterized by an X-ray powder diffractogram having one, two, three, four, five, six, seven, eight, nine, ten, or more signals selected from 4.4±0.2 degrees two-theta, 7.3±0.2 degrees two-theta, 8.9±0.2 degrees two-theta, 10.9±0.2 degrees two-theta, 11.1±0.2 degrees two-theta, 14.4±0.2 degrees two-theta, 14.7±0.2 degrees two-theta, 18.8±0.2 degrees two-theta, 21.9±0.2 degrees two-theta, 23.2±0.2 degrees two-theta, 23.8±0.2 degrees two-theta, 24.5±0.2 degrees two-theta, and 25.6±0.2 degrees two-theta.
73. The substantially crystalline Compound I heptane solvate Form B according to any one of Embodiments 70-72, characterized by an X-ray powder diffractogram having (a) one, two, three, four, five, six, seven, eight, nine, ten, or more signals selected from 4.4±0.2 degrees two-theta, 7.3±0.2 degrees two-theta, 8.9±0.2 degrees two-theta, 10.9±0.2 degrees two-theta, 11.1±0.2 degrees two-theta, 14.4±0.2 degrees two-theta, 14.7±0.2 degrees two-theta, 18.8±0.2 degrees two-theta, 21.9±0.2 degrees two-theta, 23.2±0.2 degrees two-theta, 23.8±0.2 degrees two-theta, 24.5±0.2 degrees two-theta, and 25.6±0.2 degrees two-theta, and (b) one, two, three, or four signals selected from 8.1±0.2 degrees two-theta, 10.1±0.2 degrees two-theta, 17.9±0.2 degrees two-theta, and 20.4±0.2 degrees two-theta.
74. The substantially crystalline Compound I heptane solvate Form B according to any one of Embodiments 70-73, characterized by an X-ray powder diffractogram having four, five, six, seven, eight, nine, ten, or more signals selected from 4.4±0.2 degrees two-theta, 7.3±0.2 degrees two-theta, 8.1±0.2 degrees two-theta, 8.9±0.2 degrees two-theta, 10.1±0.2 degrees two-theta, 10.9±0.2 degrees two-theta, 11.1±0.2 degrees two-theta, 14.4±0.2 degrees two-theta, 14.7±0.2 degrees two-theta, 17.9±0.2 degrees two-theta, 18.8±0.2 degrees two-theta, 20.4±0.2 degrees two-theta, 21.9±0.2 degrees two-theta, 23.2±0.2 degrees two-theta, 23.8±0.2 degrees two-theta, 24.5±0.2 degrees two-theta, and 25.6±0.2 degrees two-theta.
75. The substantially crystalline Compound I heptane solvate Form B according to any one of Embodiments 70-74, characterized by an X-ray powder diffractogram having signals at 4.4±0.2 degrees two-theta, 7.3±0.2 degrees two-theta, 8.1±0.2 degrees two-theta, 8.9±0.2 degrees two-theta, 10.1±0.2 degrees two-theta, 10.9±0.2 degrees two-theta, 11.1±0.2 degrees two-theta, 14.4±0.2 degrees two-theta, 14.7±0.2 degrees two-theta, 17.9±0.2 degrees two-theta, 18.8±0.2 degrees two-theta, 20.4±0.2 degrees two-theta, 21.9±0.2 degrees two-theta, 23.2±0.2 degrees two-theta, 23.8±0.2 degrees two-theta, 24.5±0.2 degrees two-theta, and 25.6±0.2 degrees two-theta.
76. The substantially crystalline Compound I heptane solvate Form B according to any one of Embodiments 70-75, characterized by an X-ray powder diffractogram substantially similar to FIG. 27.
77. The substantially crystalline Compound I heptane solvate Form B according to any one of Embodiments 70-76, characterized by a ¹³C SSNMR spectrum with one, two, three, four, or five peaks selected from 137.5±0.2 ppm, 126.3±0.2 ppm, 117.4±0.2 ppm, 75.5±0.2 ppm, and 34.2±0.2 ppm.
78. The substantially crystalline Compound I heptane solvate Form B according to any one of Embodiments 70-77, characterized by a ¹³C SSNMR spectrum with (a) one, two, three, four, or five peaks selected from 137.5±0.2 ppm, 126.3±0.2 ppm, 117.4±0.2 ppm, 75.5±0.2 ppm, and 34.2±0.2 ppm, and (b) one, two, three, four, five, six, seven, eight, nine, ten, or more peaks selected from 164.4±0.2 ppm, 163.0±0.2 ppm, 151.0±0.2 ppm, 139.5±0.2 ppm, 120.8±0.2 ppm, 120.0±0.2 ppm, 74.7±0.2 ppm, 74.1±0.2 ppm, 73.0±0.2 ppm, 31.1±0.2 ppm, 28.2±0.2 ppm, 22.4±0.2 ppm, 20.8±0.2 ppm, 19.5±0.2 ppm, and 13.8±0.2 ppm.
79. The substantially crystalline Compound I heptane solvate Form B according to any one of Embodiments 70-78, characterized by a ¹³C SSNMR spectrum with twelve or more peaks selected from 164.4±0.2 ppm, 163.0±0.2 ppm, 151.0±0.2 ppm, 139.5±0.2 ppm, 137.5±0.2 ppm, 126.3±0.2 ppm, 120.8±0.2 ppm, 120.0±0.2 ppm, 117.4±0.2 ppm, 75.5±0.2 ppm, 74.7±0.2 ppm, 74.1±0.2 ppm, 73.0±0.2 ppm, 34.2±0.2 ppm, 31.1±0.2 ppm, 28.2±0.2 ppm, 22.4±0.2 ppm, 20.8±0.2 ppm, 19.5±0.2 ppm, and 13.8±0.2 ppm.
80. The substantially crystalline Compound I heptane solvate Form B according to any one of Embodiments 70-79, characterized by a ¹³C SSNMR spectrum with peaks at 164.4±0.2 ppm, 163.0±0.2 ppm, 151.0±0.2 ppm, 139.5±0.2 ppm, 137.5±0.2 ppm, 126.3±0.2 ppm, 120.8±0.2 ppm, 120.0±0.2 ppm, 117.4±0.2 ppm, 75.5±0.2 ppm, 74.7±0.2 ppm, 74.1±0.2 ppm, 73.0±0.2 ppm, 34.2±0.2 ppm, 31.1±0.2 ppm, 28.2±0.2 ppm, 22.4±0.2 ppm, 20.8±0.2 ppm, 19.5±0.2 ppm, and 13.8±0.2 ppm.
81. The substantially crystalline Compound I heptane solvate Form B according to any one of Embodiments 70-80, characterized by a ¹³C SSNMR spectrum substantially similar to FIG. 29.
82. The substantially crystalline Compound I heptane solvate Form B according to any one of Embodiments 70-81, characterized by a ¹⁹F SSNMR spectrum with (a) one or two peaks selected from −78.4±0.2 ppm and −64.2±0.2 ppm, and (b) one or two peaks selected from −63.4±0.2 ppm and −77.4±0.2 ppm.
83. The substantially crystalline Compound I heptane solvate Form B according to any one of Embodiments 70-82, characterized as having a ¹⁹F SSNMR spectrum with three or four peaks selected from −78.4±0.2 ppm, −77.4±0.2 ppm, −64.2±0.2 ppm, and −63.4±0.2 ppm.
84. The substantially crystalline Compound I heptane solvate Form B according to any one of Embodiments 70-83, characterized by a ¹⁹F SSNMR spectrum substantially similar to FIG. 30.
85. Substantially crystalline Compound I heptane solvate Form C (i.e., wherein less than 15% of Compound I is in amorphous form, wherein less than 10% of Compound I is in amorphous form, wherein less than 5% of Compound I is in amorphous form).
86. The substantially crystalline Compound I heptane solvate Form C according to Embodiment 85, wherein Compound I heptane solvate Form C is 100% crystalline.
87. The substantially crystalline Compound I heptane solvate Form C according to Embodiment 85 or Embodiment 86, characterized by an X-ray powder diffractogram having one, two, or three signals selected from 9.3±0.2 degrees two-theta, 13.1±0.2 degrees two-theta, and 32.3±0.2 degrees two-theta.
88. The substantially crystalline Compound I heptane solvate Form C according to any one of Embodiments 85-87, characterized by an X-ray powder diffractogram having (a) one, two, or three signals selected from 9.3±0.2 degrees two-theta, 13.1±0.2 degrees two-theta, and 32.3±0.2 degrees two-theta, and (b) one, two, three, four, or five signals selected from 5.5±0.2 degrees two-theta, 8.0±0.2 degrees two-theta, 8.2±0.2 degrees two-theta, 11.6±0.2 degrees two-theta, and 20.4±0.2 degrees two-theta.
89. The substantially crystalline Compound I heptane solvate Form C according to any one of Embodiments 85-88, characterized by an X-ray powder diffractogram having five or six signals selected from 5.5±0.2 degrees two-theta, 8.0±0.2 degrees two-theta, 8.2±0.2 degrees two-theta, 9.3±0.2 degrees two-theta, 11.6±0.2 degrees two-theta, 13.1±0.2 degrees two-theta, 20.4±0.2 degrees two-theta, and 32.3±0.2 degrees two-theta.
90. The substantially crystalline Compound I heptane solvate Form C according to any one of Embodiments 85-89, characterized by an X-ray powder diffractogram having signals at 5.5±0.2 degrees two-theta, 8.0±0.2 degrees two-theta, 8.2±0.2 degrees two-theta, 9.3±0.2 degrees two-theta, 11.6±0.2 degrees two-theta, 13.1±0.2 degrees two-theta, 20.4±0.2 degrees two-theta, and 32.3±0.2 degrees two-theta.
91. The substantially crystalline Compound I heptane solvate Form C according to any one of Embodiments 85-90, characterized by an X-ray powder diffractogram substantially similar to FIG. 31.
92. The substantially crystalline Compound I heptane solvate Form C according to any one of Embodiments 85-91, characterized by a ¹³C SSNMR spectrum with one, two, three, four, five, six, seven, or eight peaks selected from 126.9±0.2 ppm, 124.1±0.2 ppm, 121.5±0.2 ppm, 118.8±0.2 ppm, 71.5±0.2 ppm, 36.1±0.2 ppm, 24.3±0.2 ppm, and 14.2±0.2 ppm.
93. The substantially crystalline Compound I heptane solvate Form C according to any one of Embodiments 85-92, characterized by a ¹³C SSNMR spectrum with (a) one, two, three, four, five, six, seven, or eight peaks selected from 126.9±0.2 ppm, 124.1±0.2 ppm, 121.5±0.2 ppm, 118.8±0.2 ppm, 71.5±0.2 ppm, 36.1±0.2 ppm, 24.3±0.2 ppm, and 14.2±0.2 ppm, and (b) one, two, three, four, five, six, seven, eight, nine, ten, or more peaks selected from 164.4±0.2 ppm, 163.6±0.2 ppm, 163.1±0.2 ppm, 151.1±0.2 ppm, 139.4±0.2 ppm, 128.3±0.2 ppm, 117.9±0.2 ppm, 76.1±0.2 ppm, 73.6±0.2 ppm, 37.0±0.2 ppm, 33.6±0.2 ppm, 30.9±0.2 ppm, 27.9±0.2 ppm, 23.3±0.2 ppm, 21.0±0.2 ppm, 20.0±0.2 ppm, and 18.1±0.2 ppm.
94. The substantially crystalline Compound I heptane solvate Form C according to any one of Embodiments 85-93, characterized by a ¹³C SSNMR spectrum with seventeen or more peaks selected from 164.4±0.2 ppm, 163.6±0.2 ppm, 163.1±0.2 ppm, 151.1±0.2 ppm, 139.4±0.2 ppm, 128.3±0.2 ppm, 126.9±0.2 ppm, 124.1±0.2 ppm, 121.5±0.2 ppm, 118.8±0.2 ppm, 117.9±0.2 ppm, 76.1±0.2 ppm, 73.6±0.2 ppm, 71.5±0.2 ppm, 37.0±0.2 ppm, 36.1±0.2 ppm, 33.6±0.2 ppm, 30.9±0.2 ppm, 27.9±0.2 ppm, 24.3±0.2 ppm, 23.3±0.2 ppm, 21.0±0.2 ppm, 20.0±0.2 ppm, 18.1±0.2 ppm, and 14.2±0.2 ppm.
95. The substantially crystalline Compound I heptane solvate Form C according to any one of Embodiments 85-94, characterized by a ¹³C SSNMR spectrum with peaks at 164.4±0.2 ppm, 163.6±0.2 ppm, 163.1±0.2 ppm, 151.1±0.2 ppm, 139.4±0.2 ppm, 128.3±0.2 ppm, 126.9±0.2 ppm, 124.1±0.2 ppm, 121.5±0.2 ppm, 118.8±0.2 ppm, 117.9±0.2 ppm, 76.1±0.2 ppm, 73.6±0.2 ppm, 71.5±0.2 ppm, 37.0±0.2 ppm, 36.1±0.2 ppm, 33.6±0.2 ppm, 30.9±0.2 ppm, 27.9±0.2 ppm, 24.3±0.2 ppm, 23.3±0.2 ppm, 21.0±0.2 ppm, 20.0±0.2 ppm, 18.1±0.2 ppm, and 14.2±0.2 ppm.
96. The substantially crystalline Compound I heptane solvate Form C according to any one of Embodiments 85-95, characterized by a ¹³C SSNMR spectrum substantially similar to FIG. 34.
97. Substantially crystalline Compound I octane solvate (i.e., wherein less than 15% of Compound I is in amorphous form, wherein less than 10% of Compound I is in amorphous form, wherein less than 5% of Compound I is in amorphous form).
98. The substantially crystalline Compound I octane solvate according to Embodiment 97, wherein Compound I octane solvate is 100% crystalline.
99. The substantially crystalline Compound I octane solvate according to Embodiment 97 or Embodiment 98, characterized by an X-ray powder diffractogram having one, two, three, four, or five signals selected from 5.6±0.2 degrees two-theta, 5.9±0.2 degrees two-theta, 10.2±0.2 degrees two-theta, 11.7±0.2 degrees two-theta, and 18.2±0.2 degrees two-theta.
100. The substantially crystalline Compound I octane solvate according to any one of Embodiments 97-99, characterized by an X-ray powder diffractogram having one, two, three, four, five, six, or seven signals selected from 5.6±0.2 degrees two-theta, 5.9±0.2 degrees two-theta, 10.2±0.2 degrees two-theta, 11.7±0.2 degrees two-theta, 18.2±0.2 degrees two-theta, 18.5±0.2 degrees two-theta, and 20.5±0.2 degrees two-theta.
101. The substantially crystalline Compound I octane solvate according to any one of Embodiments 97-100, characterized by an X-ray powder diffractogram substantially similar to FIG. 35.
102. The substantially crystalline Compound I octane solvate according to any one of Embodiments 97-101, characterized by a ¹³C SSNMR spectrum with one, two, three, four, five, six, seven, eight, nine, ten, or more peaks selected from 166.3±0.2 ppm, 164.6±0.2 ppm, 164.1±0.2 ppm, 153.8±0.2 ppm, 152.2±0.2 ppm, 151.7±0.2 ppm, 140.4±0.2 ppm, 137.6±0.2 ppm, 135.3±0.2 ppm, 134.8±0.2 ppm, 131.1±0.2 ppm, 130.2±0.2 ppm, 127.3±0.2 ppm, 125.5±0.2 ppm, 122.7±0.2 ppm, 120.8±0.2 ppm, 120.1±0.2 ppm, 118.1±0.2 ppm, 75.7±0.2 ppm, 74.4±0.2 ppm, 73.8±0.2 ppm, 40.2±0.2 ppm, 37.5±0.2 ppm, 36.1±0.2 ppm, 32.0±0.2 ppm, 29.9±0.2 ppm, 28.5±0.2 ppm, 27.0±0.2 ppm, 25.1±0.2 ppm, 22.4±0.2 ppm, 20.0±0.2 ppm, 17.7±0.2 ppm, 14.1±0.2 ppm, 13.5±0.2 ppm, and 12.6±0.2 ppm.
103. The substantially crystalline Compound I octane solvate according to any one of Embodiments 97-102, characterized by a ¹³C SSNMR spectrum substantially similar to FIG. 36.
104. The substantially crystalline Compound I octane solvate according to any one of Embodiments 97-103, characterized by a ¹⁹F SSNMR spectrum with one, two, three, four, five, six, seven, or eight peaks selected from −62.5±0.2 ppm, −65.0±0.2 ppm, −65.6±0.2 ppm, −66.2±0.2 ppm, −67.1±0.2 ppm, −75.1±0.2 ppm, −76.5±0.2 ppm, and −77.2±0.2 ppm.
105. The substantially crystalline Compound I octane solvate according to any one of Embodiments 97-104, characterized by a ¹⁹F SSNMR spectrum substantially similar to FIG. 37.
106. Substantially crystalline Compound I cyclohexane solvate Form A (i.e., wherein less than 15% of Compound I is in amorphous form, wherein less than 10% of Compound I is in amorphous form, wherein less than 5% of Compound I is in amorphous form).
107. The substantially crystalline Compound I cyclohexane solvate Form A according to Embodiment 106, wherein Compound I cyclohexane solvate Form A is 100% crystalline.
108. The substantially crystalline Compound I cyclohexane solvate Form A according to Embodiment 106 or Embodiment 107,
109. The substantially crystalline Compound I cyclohexane solvate Form A according to any one of Embodiments 106-108, characterized by an X-ray powder diffractogram having one, two, or three signals selected from 5.1±0.2 degrees two-theta, 16.0±0.2 degrees two-theta, and 33.6±0.2 degrees two-theta.
110. The substantially crystalline Compound I cyclohexane solvate Form A according to any one of Embodiments 106-109, characterized by an X-ray powder diffractogram having (a) one, two, or three signals selected from 5.1±0.2 degrees two-theta, 16.0±0.2 degrees two-theta, and 33.6±0.2 degrees two-theta, and (b) one, two, three, four, or five signals selected from 5.6±0.2 degrees two-theta, 16.7±0.2 degrees two-theta, 18.5±0.2 degrees two-theta, 19.9±0.2 degrees two-theta, and 21.6±0.2 degrees two-theta.
111. The substantially crystalline Compound I cyclohexane solvate Form A according to any one of Embodiments 106-110, characterized by an X-ray powder diffractogram having five, six, seven, or eight signals selected from 5.1±0.2 degrees two-theta, 5.6±0.2 degrees two-theta, 16.0±0.2 degrees two-theta, 16.7±0.2 degrees two-theta, 18.5±0.2 degrees two-theta, 19.9±0.2 degrees two-theta, 21.6±0.2 degrees two-theta, and 33.6±0.2 degrees two-theta.
112. The substantially crystalline Compound I cyclohexane solvate Form A according to any one of Embodiments 106-111, characterized by an X-ray powder diffractogram having signals at 5.1±0.2 degrees two-theta, 5.6±0.2 degrees two-theta, 16.0±0.2 degrees two-theta, 16.7±0.2 degrees two-theta, 18.5±0.2 degrees two-theta, 19.9±0.2 degrees two-theta, 21.6±0.2 degrees two-theta, and 33.6±0.2 degrees two-theta.
113. The substantially crystalline Compound I cyclohexane solvate Form A according to any one of Embodiments 106-112, characterized by an X-ray powder diffractogram substantially similar to FIG. 38.
114. The substantially crystalline Compound I cyclohexane solvate Form A according to any one of Embodiments 106-113, characterized by a ¹³C SSNMR spectrum with one, two, three, four, five, six, seven, eight, nine, or ten, or more peaks selected from 166.6±0.2 ppm, 152.1±0.2 ppm, 150.8±0.2 ppm, 140.4±0.2 ppm, 137.6±0.2 ppm, 135.4±0.2 ppm, 127.3±0.2 ppm, 125.5±0.2 ppm, 123.4±0.2 ppm, 119.7±0.2 ppm, 74.3±0.2 ppm, 37.4±0.2 ppm, 36.2±0.2 ppm, 30.6±0.2 ppm, 27.4±0.2 ppm, and 17.7±0.2 ppm.
115. The substantially crystalline Compound I cyclohexane solvate Form A according to any one of Embodiments 106-114, characterized by a ¹³C SSNMR spectrum with (a) one, two, three, four, five, six, seven, eight, nine, ten, or more peaks selected from 166.6±0.2 ppm, 152.1±0.2 ppm, 150.8±0.2 ppm, 140.4±0.2 ppm, 137.6±0.2 ppm, 135.4±0.2 ppm, 127.3±0.2 ppm, 125.5±0.2 ppm, 123.4±0.2 ppm, 119.7±0.2 ppm, 74.3±0.2 ppm, 37.4±0.2 ppm, 36.2±0.2 ppm, 30.6±0.2 ppm, 27.4±0.2 ppm, and 17.7±0.2 ppm, and (b) one, two, three, four, five, six, seven, eight, nine, ten, or more peaks selected from 164.7±0.2 ppm, 163.7±0.2 ppm, 154.4±0.2 ppm, 138.8±0.2 ppm, 131.5±0.2 ppm, 120.7±0.2 ppm, 118.1±0.2 ppm, 75.7±0.2 ppm, 73.4±0.2 ppm, 21.9±0.2 ppm, and 19.4±0.2 ppm.
116. The substantially crystalline Compound I cyclohexane solvate Form A according to any one of Embodiments 106-115, characterized by a ¹³C SSNMR spectrum with twelve or more peaks selected from 166.6±0.2 ppm, 164.7±0.2 ppm, 163.7±0.2 ppm, 154.4±0.2 ppm, 152.1±0.2 ppm, 150.8±0.2 ppm, 140.4±0.2 ppm, 138.8±0.2 ppm, 137.6±0.2 ppm, 135.4±0.2 ppm, 131.5±0.2 ppm, 127.3±0.2 ppm, 125.5±0.2 ppm, 123.4±0.2 ppm, 120.7±0.2 ppm, 119.7±0.2 ppm, 118.1±0.2 ppm, 75.7±0.2 ppm, 74.3±0.2 ppm, 73.4±0.2 ppm, 37.4±0.2 ppm, 36.2±0.2 ppm, 30.6±0.2 ppm, 27.4±0.2 ppm, 21.9±0.2 ppm, 19.4±0.2 ppm, and 17.7±0.2 ppm.
117. The substantially crystalline Compound I cyclohexane solvate Form A according to any one of Embodiments 106-116, characterized by a ¹³C SSNMR spectrum with peaks at 166.6±0.2 ppm, 164.7±0.2 ppm, 163.7±0.2 ppm, 154.4±0.2 ppm, 152.1±0.2 ppm, 150.8±0.2 ppm, 140.4±0.2 ppm, 138.8±0.2 ppm, 137.6±0.2 ppm, 135.4±0.2 ppm, 131.5±0.2 ppm, 127.3±0.2 ppm, 125.5±0.2 ppm, 123.4±0.2 ppm, 120.7±0.2 ppm, 119.7±0.2 ppm, 118.1±0.2 ppm, 75.7±0.2 ppm, 74.3±0.2 ppm, 73.4±0.2 ppm, 37.4±0.2 ppm, 36.2±0.2 ppm, 30.6±0.2 ppm, 27.4±0.2 ppm, 21.9±0.2 ppm, 19.4±0.2 ppm, and 17.7±0.2 ppm.
118. The substantially crystalline Compound I cyclohexane solvate Form A according to any one of Embodiments 106-117, characterized by a ¹³C SSNMR spectrum substantially similar to FIG. 39.
119. The substantially crystalline Compound I cyclohexane solvate Form A according to any one of Embodiments 106-118, characterized by a ¹⁹F SSNMR spectrum with one, two, three, four, or five peaks selected from −62.6±0.2 ppm, −65.9±0.2 ppm, −66.8±0.2 ppm, −75.4±0.2 ppm, and −77.6±0.2 ppm.
120. The substantially crystalline Compound I cyclohexane solvate Form A according to any one of Embodiments 106-119, characterized by a ¹⁹F SSNMR spectrum with (a) one, two, three, four, or five peaks selected from −62.6±0.2 ppm, −65.9±0.2 ppm, −66.8±0.2 ppm, −75.4±0.2 ppm, and −77.6±0.2 ppm, and (b) one or two peaks selected from −64.5±0.2 ppm and −76.6±0.2 ppm.
121. The substantially crystalline Compound I cyclohexane solvate Form A according to any one of Embodiments 106-120, characterized by a ¹⁹F SSNMR spectrum with three, four, five, six, or seven peaks selected from −62.6±0.2 ppm, −64.5±0.2 ppm, −65.9±0.2 ppm, −66.8±0.2 ppm, −75.4±0.2 ppm, −76.6±0.2 ppm, and −77.6±0.2 ppm.
122. The substantially crystalline Compound I cyclohexane solvate Form A according to any one of Embodiments 106-121, characterized by a ¹⁹F SSNMR spectrum with peaks at −62.6±0.2 ppm, −64.5±0.2 ppm, −65.9±0.2 ppm, −66.8±0.2 ppm, −75.4±0.2 ppm, −76.6±0.2 ppm, and −77.6±0.2 ppm.
123. The substantially crystalline Compound I cyclohexane solvate Form A according to any one of Embodiments 106-122, characterized by a ¹⁹F SSNMR spectrum substantially similar to FIG. 40.
124. Substantially crystalline Compound I cyclohexane solvate Form B (i.e., wherein less than 15% of Compound I is in amorphous form, wherein less than 10% of Compound I is in amorphous form, wherein less than 5% of Compound I is in amorphous form).
125. The substantially crystalline Compound I cyclohexane solvate Form B according to Embodiment 124, wherein Compound I cyclohexane solvate Form B is 100% crystalline.
126. The substantially crystalline Compound I cyclohexane solvate Form B according to Embodiment 124 or Embodiment 125, characterized by an X-ray powder diffractogram having one, two, three, or four signals selected from 15.5±0.2 degrees two-theta, 20.8±0.2 degrees two-theta, 23.4±0.2 degrees two-theta, and 26.7±0.2 degrees two-theta.
127. The substantially crystalline Compound I cyclohexane solvate Form B according to any one of Embodiments 124-126, characterized by an X-ray powder diffractogram having (a) one, two, three, or four signals selected from 15.5±0.2 degrees two-theta, 20.8±0.2 degrees two-theta, 23.4±0.2 degrees two-theta, and 26.7±0.2 degrees two-theta, and (b) one, two, three, four, five, six, or seven signals selected from 7.8±0.2 degrees two-theta, 11.8±0.2 degrees two-theta, 13.8±0.2 degrees two-theta, 16.6±0.2 degrees two-theta, 18.4±0.2 degrees two-theta, 19.9±0.2 degrees two-theta, and 21.6±0.2 degrees two-theta.
128. The substantially crystalline Compound I cyclohexane solvate Form B according to any one of Embodiments 124-127, characterized by an X-ray powder diffractogram having five, six, seven, eight, nine, ten, or more signals selected from 7.8±0.2 degrees two-theta, 11.8±0.2 degrees two-theta, 13.8±0.2 degrees two-theta, 15.5±0.2 degrees two-theta, 16.6±0.2 degrees two-theta, 18.4±0.2 degrees two-theta, 19.9±0.2 degrees two-theta, 20.8±0.2 degrees two-theta, 21.6±0.2 degrees two-theta, 23.4±0.2 degrees two-theta, and 26.7±0.2 degrees two-theta.
129. The substantially crystalline Compound I cyclohexane solvate Form B according to any one of Embodiments 124-128, characterized by an X-ray powder diffractogram having signals at 7.8±0.2 degrees two-theta, 11.8±0.2 degrees two-theta, 13.8±0.2 degrees two-theta, 15.5±0.2 degrees two-theta, 16.6±0.2 degrees two-theta, 18.4±0.2 degrees two-theta, 19.9±0.2 degrees two-theta, 20.8±0.2 degrees two-theta, 21.6±0.2 degrees two-theta, 23.4±0.2 degrees two-theta, and 26.7±0.2 degrees two-theta.
130. The substantially crystalline Compound I cyclohexane solvate Form B according to any one of Embodiments 124-129, characterized by an X-ray powder diffractogram substantially similar to FIG. 41.
131. The substantially crystalline Compound I cyclohexane solvate Form B according to any one of Embodiments 124-130, characterized by a ¹³C SSNMR spectrum with one, two, three, four, or five peaks selected from 128.0±0.2 ppm, 34.7±0.2 ppm, 31.5±0.2 ppm, 26.5±0.2 ppm, and 19.0±0.2 ppm.
132. The substantially crystalline Compound I cyclohexane solvate Form B according to any one of Embodiments 124-131, characterized by a ¹³C SSNMR spectrum with (a) one, two, three, four, or five peaks selected from 128.0±0.2 ppm, 34.7±0.2 ppm, 31.5±0.2 ppm, 26.5±0.2 ppm, and 19.0±0.2 ppm, and (b) one, two, three, four, five, six, seven, eight, nine, ten, or more peaks selected from 164.7±0.2 ppm, 150.9±0.2 ppm, 138.7±0.2 ppm, 118.2±0.2 ppm, 75.6±0.2 ppm, 73.6±0.2 ppm, 36.5±0.2 ppm, and 19.5±0.2 ppm.
133. The substantially crystalline Compound I cyclohexane solvate Form B according to any one of Embodiments 124-132, characterized by a ¹³C SSNMR spectrum with nine, ten, or more peaks selected from 164.7±0.2 ppm, 150.9±0.2 ppm, 138.7±0.2 ppm, 128.0±0.2 ppm, 118.2±0.2 ppm, 75.6±0.2 ppm, 73.6±0.2 ppm, 36.5±0.2 ppm, 34.7±0.2 ppm, 31.5±0.2 ppm, 26.5±0.2 ppm, 19.5±0.2 ppm, and 19.0±0.2 ppm.
134. The substantially crystalline Compound I cyclohexane solvate Form B according to any one of Embodiments 124-133, characterized by a ¹³C SSNMR spectrum with peaks at 164.7±0.2 ppm, 150.9±0.2 ppm, 138.7±0.2 ppm, 128.0±0.2 ppm, 118.2±0.2 ppm, 75.6±0.2 ppm, 73.6±0.2 ppm, 36.5±0.2 ppm, 34.7±0.2 ppm, 31.5±0.2 ppm, 26.5±0.2 ppm, 19.5±0.2 ppm, and 19.0±0.2 ppm.
135. The substantially crystalline Compound I cyclohexane solvate Form B according to any one of Embodiments 124-134, characterized by a ¹³C SSNMR spectrum substantially similar to FIG. 43.
136. The substantially crystalline Compound I cyclohexane solvate Form B according to any one of Embodiments 124-135, characterized by a ¹⁹F SSNMR spectrum with a peak at −75.0±0.2 ppm.
137. The substantially crystalline Compound I cyclohexane solvate Form B according to any one of Embodiments 124-136, characterized by a ¹⁹F SSNMR spectrum with peaks at −64.3±0.2 ppm and −75.0±0.2 ppm.
138. The substantially crystalline Compound I cyclohexane solvate Form B according to any one of Embodiments 124-137, characterized by a ¹⁹F SSNMR spectrum substantially similar to FIG. 44.
139. Substantially crystalline Compound I cyclohexane solvate Form C (i.e., wherein less than 15% of Compound I is in amorphous form, wherein less than 10% of Compound I is in amorphous form, wherein less than 5% of Compound I is in amorphous form).
140. The substantially crystalline Compound I cyclohexane solvate Form C according to Embodiment 139, wherein Compound I cyclohexane solvate Form C is 100% crystalline.
141. The substantially crystalline Compound I cyclohexane solvate Form C according to Embodiment 139 or Embodiment 140, characterized by an X-ray powder diffractogram having a signal at 10.0±0.2 degrees two-theta.
142. The substantially crystalline Compound I cyclohexane solvate Form C according to any one of Embodiments 139-141, characterized by an X-ray powder diffractogram having (a) a signal at 10.0±0.2 degrees two-theta, and (b) one, two, three, four, or five signals selected from 5.8±0.2 degrees two-theta, 7.8±0.2 degrees two-theta, 11.8±0.2 degrees two-theta, 13.9±0.2 degrees two-theta, and 19.9±0.2 degrees two-theta.
143. The substantially crystalline Compound I cyclohexane solvate Form C according to any one of Embodiments 139-142, characterized by an X-ray powder diffractogram having signals at 5.8±0.2 degrees two-theta, 7.8±0.2 degrees two-theta, 10.0±0.2 degrees two-theta, 11.8±0.2 degrees two-theta, 13.9±0.2 degrees two-theta, and 19.9±0.2 degrees two-theta.
144. The substantially crystalline Compound I cyclohexane solvate Form C according to any one of Embodiments 139-143, characterized by an X-ray powder diffractogram substantially similar to FIG. 45.
145. Substantially crystalline Compound I ethanol solvate (i.e., wherein less than 15% of Compound I is in amorphous form, wherein less than 10% of Compound I is in amorphous form, wherein less than 5% of Compound I is in amorphous form).
146. The substantially crystalline Compound I ethanol solvate according to Embodiment 145, wherein Compound I ethanol solvate is 100% crystalline.
147. The substantially crystalline Compound I ethanol solvate according to Embodiment 145 or Embodiment 146, characterized by an X-ray powder diffractogram having one, two, or three signals selected from 6.2±0.2 degrees two-theta, 7.8±0.2 degrees two-theta, and 13.3±0.2 degrees two-theta.
148. The substantially crystalline Compound I ethanol solvate according to any one of Embodiments 145-147, characterized by an X-ray powder diffractogram substantially similar to FIG. 46.
149. The substantially crystalline Compound I ethanol solvate according to any one of Embodiments 145-148, characterized by a ¹³C SSNMR spectrum with one, two, three, four, five, six, seven, eight, nine, ten, or more peaks selected from 162.8±0.2 ppm, 151.7±0.2 ppm, 150.7±0.2 ppm, 139.1±0.2 ppm, 138.0±0.2 ppm, 127.4±0.2 ppm, 126.9±0.2 ppm, 124.3±0.2 ppm, 120.4±0.2 ppm, 117.7±0.2 ppm, 78.7±0.2 ppm, 77.9±0.2 ppm, 72.6±0.2 ppm, 33.4±0.2 ppm, 25.9±0.2 ppm, 21.7±0.2 ppm, 20.0±0.2 ppm, 18.8±0.2 ppm, and 17.9±0.2 ppm.
150. The substantially crystalline Compound I ethanol solvate according to any one of Embodiments 145-149, characterized by a ¹³C SSNMR spectrum substantially similar to FIG. 47.
151. The substantially crystalline Compound I ethanol solvate according to any one of Embodiments 145-150, characterized by a ¹⁹F SSNMR spectrum with one, two, or three peaks selected from −63.1±0.2 ppm, −64.2±0.2 ppm, and −78.0±0.2 ppm.
152. The substantially crystalline Compound I ethanol solvate according to any one of Embodiments 145-151, characterized by a ¹⁹F SSNMR spectrum substantially similar to FIG. 48.
153. Substantially crystalline Compound I solvate/hydrate (dry) (i.e., wherein less than 15% of Compound I is in amorphous form, wherein less than 10% of Compound I is in amorphous form, wherein less than 5% of Compound I is in amorphous form).
154. The substantially crystalline Compound I solvate/hydrate (dry) according to Embodiment 153, wherein Compound I solvate/hydrate (dry) is 100% crystalline.
155. The substantially crystalline Compound I solvate/hydrate (dry) according to Embodiment 153 or Embodiment 154, characterized by an X-ray powder diffractogram having a signal at 22.7±0.2 degrees two-theta.
156. The substantially crystalline Compound I solvate/hydrate (dry) according to any one of Embodiments 153-155, characterized by an X-ray powder diffractogram having (a) a signal at 22.7±0.2 degrees two-theta, and (b) one, two, three, four, five, six, seven, eight, nine, ten, or more signals selected from 4.4±0.2 degrees two-theta, 8.8±0.2 degrees two-theta, 10.3±0.2 degrees two-theta, 11.3±0.2 degrees two-theta, 11.7±0.2 degrees two-theta, 13.4±0.2 degrees two-theta, 14.1±0.2 degrees two-theta, 15.1±0.2 degrees two-theta, 17.7±0.2 degrees two-theta, 18.1±0.2 degrees two-theta, 18.9±0.2 degrees two-theta, 20.6±0.2 degrees two-theta, 21.2±0.2 degrees two-theta, 22.3±0.2 degrees two-theta, 22.9±0.2 degrees two-theta, 23.3±0.2 degrees two-theta, 27.0±0.2 degrees two-theta, and 28.3±0.2 degrees two-theta.
157. The substantially crystalline Compound I solvate/hydrate (dry) according to any one of Embodiments 153-156, characterized by an X-ray powder diffractogram having signals at 4.4±0.2 degrees two-theta, 8.8±0.2 degrees two-theta, 10.3±0.2 degrees two-theta, 11.3±0.2 degrees two-theta, 11.7±0.2 degrees two-theta, 13.4±0.2 degrees two-theta, 14.1±0.2 degrees two-theta, 15.1±0.2 degrees two-theta, 17.7±0.2 degrees two-theta, 18.1±0.2 degrees two-theta, 18.9±0.2 degrees two-theta, 20.6±0.2 degrees two-theta, 21.2±0.2 degrees two-theta, 22.3±0.2 degrees two-theta, 22.7±0.2 degrees two-theta, 22.9±0.2 degrees two-theta, 23.3±0.2 degrees two-theta, 27.0±0.2 degrees two-theta, and 28.3±0.2 degrees two-theta
158. The substantially crystalline Compound I solvate/hydrate (dry) according to any one of Embodiments 153-157, characterized by an X-ray powder diffractogram substantially similar to FIG. 49.
159. Substantially crystalline Compound I solvate/hydrate (wet) (i.e., wherein less than 15% of Compound I is in amorphous form, wherein less than 10% of Compound I is in amorphous form, wherein less than 5% of Compound I is in amorphous form).
160. The substantially crystalline Compound I solvate/hydrate (wet) according to Embodiment 159, wherein Compound I solvate/hydrate (wet) is 100% crystalline.
161. The substantially crystalline Compound I solvate/hydrate (wet) according to Embodiment 159 or Embodiment 160, characterized by an X-ray powder diffractogram having a signal at 26.4±0.2 degrees two-theta.
162. The substantially crystalline Compound I solvate/hydrate (wet) according to any one of Embodiments 159-161, characterized by an X-ray powder diffractogram having (a) a signal at 26.4±0.2 degrees two-theta, and (b) one, two, three, four, five, six, seven, eight, nine, ten, or more signals selected from 4.4±0.2 degrees two-theta, 8.7±0.2 degrees two-theta, 10.2±0.2 degrees two-theta, 11.3±0.2 degrees two-theta, 11.7±0.2 degrees two-theta, 13.4±0.2 degrees two-theta, 14.1±0.2 degrees two-theta, 15.0±0.2 degrees two-theta, 17.6±0.2 degrees two-theta, 18.1±0.2 degrees two-theta, 18.8±0.2 degrees two-theta, 19.0±0.2 degrees two-theta, 20.4±0.2 degrees two-theta, 20.9±0.2 degrees two-theta, 21.1±0.2 degrees two-theta, 22.1±0.2 degrees two-theta, 22.3±0.2 degrees two-theta, 23.0±0.2 degrees two-theta, 27.0±0.2 degrees two-theta, and 28.3±0.2 degrees two-theta.
163. The substantially crystalline Compound I solvate/hydrate (wet) according to any one of Embodiments 159-162, characterized by an X-ray powder diffractogram having signals at 4.4±0.2 degrees two-theta, 8.7±0.2 degrees two-theta, 10.2±0.2 degrees two-theta, 11.3±0.2 degrees two-theta, 11.7±0.2 degrees two-theta, 13.4±0.2 degrees two-theta, 14.1±0.2 degrees two-theta, 15.0±0.2 degrees two-theta, 17.6±0.2 degrees two-theta, 18.1±0.2 degrees two-theta, 18.8±0.2 degrees two-theta, 19.0±0.2 degrees two-theta, 20.4±0.2 degrees two-theta, 20.9±0.2 degrees two-theta, 21.1±0.2 degrees two-theta, 22.1±0.2 degrees two-theta, 22.3±0.2 degrees two-theta, 23.0±0.2 degrees two-theta, 26.4±0.2 degrees two-theta, 27.0±0.2 degrees two-theta, and 28.3±0.2 degrees two-theta.
164. The substantially crystalline Compound I solvate/hydrate (wet) according to any one of Embodiments 159-163, characterized by an X-ray powder diffractogram substantially similar to FIG. 52.
165. The substantially crystalline Compound I solvate/hydrate (wet) according to any one of Embodiments 159-164, characterized as having a ¹³C SSNMR spectrum with one, two, three, four, five, six, seven, eight, nine, ten, or more peaks selected from 163.5±0.2 ppm, 162.4±0.2 ppm, 151.7±0.2 ppm, 139.2±0.2 ppm, 137.8±0.2 ppm, 128.3±0.2 ppm, 126.4±0.2 ppm, 124.4±0.2 ppm, 122.2±0.2 ppm, 118.4±0.2 ppm, 116.8±0.2 ppm, 77.8±0.2 ppm, 77.6±0.2 ppm, 72.9±0.2 ppm, 72.5±0.2 ppm, 36.9±0.2 ppm, 35.6±0.2 ppm, 33.9±0.2 ppm, 25.6±0.2 ppm, 25.2±0.2 ppm, 22.5±0.2 ppm, 21.0±0.2 ppm, 20.0±0.2 ppm, and 17.2±0.2 ppm.
166. The substantially crystalline Compound I solvate/hydrate (wet) according to any one of Embodiments 159-165, characterized by a ¹³C SSNMR spectrum substantially similar to FIG. 53.
167. The substantially crystalline Compound I solvate/hydrate (wet) according to any one of Embodiments 159-166, characterized by a ¹⁹F SSNMR spectrum with one, two, three, or four peaks selected from −62.3±0.2 ppm, −64.5±0.2 ppm, −76.1±0.2 ppm, and −78.2±0.2 ppm.
168. The substantially crystalline Compound I solvate/hydrate (wet) according to any one of Embodiments 159-167, characterized by a ¹⁹F SSNMR spectrum substantially similar to FIG. 54.
169. Substantially crystalline Compound I L-lysine cocrystal (i.e., wherein less than 15% of Compound I is in amorphous form, wherein less than 10% of Compound I is in amorphous form, wherein less than 5% of Compound I is in amorphous form).
170. The substantially crystalline Compound I L-lysine cocrystal according to Embodiment 169, wherein Compound I L-lysine cocrystal is 100% crystalline.
171. The substantially crystalline Compound I L-lysine cocrystal according to Embodiment 169 or Embodiment 170, characterized by an X-ray powder diffractogram having one, two, three, four, or five signals selected from 7.9±0.2 degrees two-theta, 10.5±0.2 degrees two-theta, 19.9±0.2 degrees two-theta, 21.1±0.2 degrees two-theta, and 21.6±0.2 degrees two-theta.
172. The substantially crystalline Compound I L-lysine cocrystal according to any one of Embodiments 169-171, characterized by an X-ray powder diffractogram having one, two, three, four, five, six, seven, eight, nine, or ten signals selected from 7.9±0.2 degrees two-theta, 9.5±0.2 degrees two-theta, 10.5±0.2 degrees two-theta, 11.4±0.2 degrees two-theta, 17.8±0.2 degrees two-theta, 19.9±0.2 degrees two-theta, 20.8±0.2 degrees two-theta, 21.1±0.2 degrees two-theta, 21.6±0.2 degrees two-theta, and 22.9±0.2 degrees two-theta.
173. The substantially crystalline Compound I L-lysine cocrystal according to any one of Embodiments 169-172, characterized by an X-ray powder diffractogram having one, two, three, four, five, six, seven, eight, nine, ten, or more signals selected from 3.9±0.2 degrees two-theta, 7.9±0.2 degrees two-theta, 8.9±0.2 degrees two-theta, 9.5±0.2 degrees two-theta, 10.5±0.2 degrees two-theta, 11.4±0.2 degrees two-theta, 11.8±0.2 degrees two-theta, 13.3±0.2 degrees two-theta, 13.4±0.2 degrees two-theta, 13.8±0.2 degrees two-theta, 15.4±0.2 degrees two-theta, 15.9±0.2 degrees two-theta, 16.4±0.2 degrees two-theta, 17.5±0.2 degrees two-theta, 17.8±0.2 degrees two-theta, 18.2±0.2 degrees two-theta, 18.6±0.2 degrees two-theta, 19.2±0.2 degrees two-theta, 19.9±0.2 degrees two-theta, 20.8±0.2 degrees two-theta, 21.1±0.2 degrees two-theta, 21.6±0.2 degrees two-theta, 22.3±0.2 degrees two-theta, 22.9±0.2 degrees two-theta, 23.6±0.2 degrees two-theta, 26.3±0.2 degrees two-theta, 26.6±0.2 degrees two-theta, 27.0±0.2 degrees two-theta, 27.5±0.2 degrees two-theta, 29.2±0.2 degrees two-theta, and 29.7±0.2 degrees two-theta.
174. The substantially crystalline Compound I L-lysine cocrystal according to any one of Embodiments 169-173, characterized by an X-ray powder diffractogram having signals at 3.9±0.2 degrees two-theta, 7.9±0.2 degrees two-theta, 8.9±0.2 degrees two-theta, 9.5±0.2 degrees two-theta, 10.5±0.2 degrees two-theta, 11.4±0.2 degrees two-theta, 11.8±0.2 degrees two-theta, 13.3±0.2 degrees two-theta, 13.4±0.2 degrees two-theta, 13.8±0.2 degrees two-theta, 15.4±0.2 degrees two-theta, 15.9±0.2 degrees two-theta, 16.4±0.2 degrees two-theta, 17.5±0.2 degrees two-theta, 17.8±0.2 degrees two-theta, 18.2±0.2 degrees two-theta, 18.6±0.2 degrees two-theta, 19.2±0.2 degrees two-theta, 19.9±0.2 degrees two-theta, 20.8±0.2 degrees two-theta, 21.1±0.2 degrees two-theta, 21.6±0.2 degrees two-theta, 22.3±0.2 degrees two-theta, 22.9±0.2 degrees two-theta, 23.6±0.2 degrees two-theta, 26.3±0.2 degrees two-theta, 26.6±0.2 degrees two-theta, 27.0±0.2 degrees two-theta, 27.5±0.2 degrees two-theta, 29.2±0.2 degrees two-theta, and 29.7±0.2 degrees two-theta.
175. The substantially crystalline Compound I L-lysine cocrystal according to any one of Embodiments 169-174, characterized by an X-ray powder diffractogram substantially similar to FIG. 55.
176. The substantially crystalline Compound I L-lysine cocrystal according to any one of Embodiments 169-175, characterized by a ¹³C SSNMR spectrum with one, two, three, four, five, six, seven, eight, nine, ten, or more peaks selected from 181.6±0.2 ppm, 180.9±0.2 ppm, 177.5±0.2 ppm, 165.4±0.2 ppm, 164.4±0.2 ppm, 163.7±0.2 ppm, 162.7±0.2 ppm, 151.9±0.2 ppm, 150.7±0.2 ppm, 138.9±0.2 ppm, 138.2±0.2 ppm, 127.6±0.2 ppm, 126.8±0.2 ppm, 125.8±0.2 ppm, 124.1±0.2 ppm, 121.4±0.2 ppm, 119.6±0.2 ppm, 118.0±0.2 ppm, 78.4±0.2 ppm, 77.1±0.2 ppm, 75.9±0.2 ppm, 73.1±0.2 ppm, 56.8±0.2 ppm, 54.9±0.2 ppm, 45.1±0.2 ppm, 43.6±0.2 ppm, 41.4±0.2 ppm, 39.6±0.2 ppm, 38.8±0.2 ppm, 37.0±0.2 ppm, 34.3±0.2 ppm, 33.4±0.2 ppm, 32.2±0.2 ppm, 31.6±0.2 ppm, 30.6±0.2 ppm, 29.2±0.2 ppm, 27.4±0.2 ppm, 25.8±0.2 ppm, 25.1±0.2 ppm, 22.9±0.2 ppm, 22.5±0.2 ppm, 21.7±0.2 ppm, 20.5±0.2 ppm, 19.4±0.2 ppm, and 18.6±0.2 ppm.
177. The substantially crystalline Compound I L-lysine cocrystal according to any one of Embodiments 169-176, characterized by a ¹³C SSNMR spectrum with peaks at 181.6±0.2 ppm, 180.9±0.2 ppm, 177.5±0.2 ppm, 165.4±0.2 ppm, 164.4±0.2 ppm, 163.7±0.2 ppm, 162.7±0.2 ppm, 151.9±0.2 ppm, 150.7±0.2 ppm, 138.9±0.2 ppm, 138.2±0.2 ppm, 127.6±0.2 ppm, 126.8±0.2 ppm, 125.8±0.2 ppm, 124.1±0.2 ppm, 121.4±0.2 ppm, 119.6±0.2 ppm, 118.0±0.2 ppm, 78.4±0.2 ppm, 77.1±0.2 ppm, 75.9±0.2 ppm, 73.1±0.2 ppm, 56.8±0.2 ppm, 54.9±0.2 ppm, 45.1±0.2 ppm, 43.6±0.2 ppm, 41.4±0.2 ppm, 39.6±0.2 ppm, 38.8±0.2 ppm, 37.0±0.2 ppm, 34.3±0.2 ppm, 33.4±0.2 ppm, 32.2±0.2 ppm, 31.6±0.2 ppm, 30.6±0.2 ppm, 29.2±0.2 ppm, 27.4±0.2 ppm, 25.8±0.2 ppm, 25.1±0.2 ppm, 22.9±0.2 ppm, 22.5±0.2 ppm, 21.7±0.2 ppm, 20.5±0.2 ppm, 19.4±0.2 ppm, and 18.6±0.2 ppm.
178. The substantially crystalline Compound I L-lysine cocrystal according to any one of Embodiments 169-177, characterized by a ¹³C SSNMR spectrum substantially similar to FIG. 58.
179. Substantially crystalline Compound I L-arginine cocrystal (i.e., wherein less than 15% of Compound I is in amorphous form, wherein less than 10% of Compound I is in amorphous form, wherein less than 5% of Compound I is in amorphous form).
180. The substantially crystalline Compound I L-arginine cocrystal according to Embodiment 179, wherein Compound I L-arginine cocrystal is 100% crystalline.
181. The substantially crystalline Compound I L-arginine cocrystal according to Embodiment 179 or Embodiment 180, characterized by an X-ray powder diffractogram having one, two, three, four, or five signals selected from 7.5±0.2 degrees two-theta, 18.3±0.2 degrees two-theta, 19.1±0.2 degrees two-theta, 19.4±0.2 degrees two-theta, and 23.1±0.2 degrees two-theta.
182. The substantially crystalline Compound I L-arginine cocrystal according to any one of Embodiments 179-181, characterized by an X-ray powder diffractogram having one, two, three, four, five, six, seven, eight, nine, or ten signals selected from 7.5±0.2 degrees two-theta, 9.0±0.2 degrees two-theta, 10.5±0.2 degrees two-theta, 18.3±0.2 degrees two-theta, 19.1±0.2 degrees two-theta, 19.4±0.2 degrees two-theta, 21±0.2 degrees two-theta, 21.9±0.2 degrees two-theta, 23.1±0.2 degrees two-theta, and 27.4±0.2 degrees two-theta.
183. The substantially crystalline Compound I L-arginine cocrystal according to any one of Embodiments 179-182, characterized by an X-ray powder diffractogram having signals at one, two, three, four, five, six, seven, eight, nine, ten, or more of 7.5±0.2 degrees two-theta, 9.0±0.2 degrees two-theta, 10.5±0.2 degrees two-theta, 13.4±0.2 degrees two-theta, 15.9±0.2 degrees two-theta, 18.3±0.2 degrees two-theta, 19.1±0.2 degrees two-theta, 19.4±0.2 degrees two-theta, 21.0±0.2 degrees two-theta, 21.9±0.2 degrees two-theta, 23.1±0.2 degrees two-theta, and 27.4±0.2 degrees two-theta.
184. The substantially crystalline Compound I L-arginine cocrystal according to any one of Embodiments 179-183, characterized by an X-ray powder diffractogram having signals at 7.5±0.2 degrees two-theta, 9.0±0.2 degrees two-theta, 10.5±0.2 degrees two-theta, 13.4±0.2 degrees two-theta, 15.9±0.2 degrees two-theta, 18.3±0.2 degrees two-theta, 19.1±0.2 degrees two-theta, 19.4±0.2 degrees two-theta, 21.0±0.2 degrees two-theta, 21.9±0.2 degrees two-theta, 23.1±0.2 degrees two-theta, and 27.4±0.2 degrees two-theta.
185. The substantially crystalline Compound I L-arginine cocrystal according to any one of Embodiments 179-184, characterized by an X-ray powder diffractogram substantially similar to FIG. 59.
186. Substantially crystalline Compound I L-phenylalanine cocrystal (i.e., wherein less than 15% of Compound I is in amorphous form, wherein less than 10% of Compound I is in amorphous form, wherein less than 5% of Compound I is in amorphous form).
187. The substantially crystalline Compound I L-phenylalanine cocrystal according to Embodiment 186, wherein Compound I L-phenylalanine cocrystal is 100% crystalline.
188. The substantially crystalline Compound I L-phenylalanine cocrystal according to Embodiment 186 or Embodiment 187, characterized by an X-ray powder diffractogram having one, two, three, four, or five signals selected from 6.5±0.2 degrees two-theta, 10.1±0.2 degrees two-theta, 11.1±0.2 degrees two-theta, 17.6±0.2 degrees two-theta, and 20.5±0.2 degrees two-theta.
189. The substantially crystalline Compound I L-phenylalanine cocrystal according to any one of Embodiments 186-188, characterized by an X-ray powder diffractogram having one, two, three, four, five, six, seven, eight, nine, or ten signals selected from 6.5±0.2 degrees two-theta, 10.1±0.2 degrees two-theta, 11.1±0.2 degrees two-theta, 14.8±0.2 degrees two-theta, 15.3±0.2 degrees two-theta, 17.6±0.2 degrees two-theta, 18.4±0.2 degrees two-theta, 19.8±0.2 degrees two-theta, 20.5±0.2 degrees two-theta, and 21.4±0.2 degrees two-theta.
190. The substantially crystalline Compound I L-phenylalanine cocrystal according to any one of Embodiments 186-189, characterized by an X-ray powder diffractogram having one, two, three, four, five, six, seven, eight, nine, ten, or more signals selected from 4.9±0.2 degrees two-theta, 6.5±0.2 degrees two-theta, 7.4±0.2 degrees two-theta, 9.0±0.2 degrees two-theta, 10.1±0.2 degrees two-theta, 11.1±0.2 degrees two-theta, 14.8±0.2 degrees two-theta, 15.3±0.2 degrees two-theta, 16.2±0.2 degrees two-theta, 17.6±0.2 degrees two-theta, 18.4±0.2 degrees two-theta, 19.8±0.2 degrees two-theta, 20.5±0.2 degrees two-theta, 21.4±0.2 degrees two-theta, 22.2±0.2 degrees two-theta, 22.9±0.2 degrees two-theta, 23.9±0.2 degrees two-theta, 26.3±0.2 degrees two-theta, and 27.9±0.2 degrees two-theta.
191. The substantially crystalline Compound I L-phenylalanine cocrystal according to any one of Embodiments 186-190, characterized by an X-ray powder diffractogram having signals at 4.9±0.2 degrees two-theta, 6.5±0.2 degrees two-theta, 7.4±0.2 degrees two-theta, 9.0±0.2 degrees two-theta, 10.1±0.2 degrees two-theta, 11.1±0.2 degrees two-theta, 14.8±0.2 degrees two-theta, 15.3±0.2 degrees two-theta, 16.2±0.2 degrees two-theta, 17.6±0.2 degrees two-theta, 18.4±0.2 degrees two-theta, 19.8±0.2 degrees two-theta, 20.5±0.2 degrees two-theta, 21.4±0.2 degrees two-theta, 22.2±0.2 degrees two-theta, 22.9±0.2 degrees two-theta, 23.9±0.2 degrees two-theta, 26.3±0.2 degrees two-theta, and 27.9±0.2 degrees two-theta.
192. The substantially crystalline Compound I L-phenylalanine cocrystal according to any one of Embodiments 186-191, characterized by an X-ray powder diffractogram substantially similar to FIG. 62.
193. Substantially crystalline Compound I succinic acid cocrystal (wet) (i.e., wherein less than 15% of Compound I is in amorphous form, wherein less than 10% of Compound I is in amorphous form, wherein less than 5% of Compound I is in amorphous form).
194. The substantially crystalline Compound I succinic acid cocrystal (wet) according to Embodiment 193, wherein Compound I succinic acid cocrystal (wet) is 100% crystalline.
195. The substantially crystalline Compound I succinic acid cocrystal (wet) according to Embodiment 193 or Embodiment 194, characterized by an X-ray powder diffractogram having one, two, three, four, or five signals selected from 4.0±0.2 degrees two-theta, 13.5±0.2 degrees two-theta, 14.4±0.2 degrees two-theta, 21.7±0.2 degrees two-theta, and 22.0±0.2 degrees two-theta.
196. The substantially crystalline Compound I succinic acid cocrystal (wet) according to any one of Embodiments 193-195, characterized by an X-ray powder diffractogram having one, two, three, four, five, six, seven, eight, nine, or ten signals selected from 4.0±0.2 degrees two-theta, 8.1±0.2 degrees two-theta, 9.1±0.2 degrees two-theta, 12.1±0.2 degrees two-theta, 13.5±0.2 degrees two-theta, 14.4±0.2 degrees two-theta, 20.4±0.2 degrees two-theta, 21.7±0.2 degrees two-theta, 22.0±0.2 degrees two-theta, and 27.1±0.2 degrees two-theta.
197. The substantially crystalline Compound I succinic acid cocrystal (wet) according to any one of Embodiments 193-196, characterized by an X-ray powder diffractogram having one, two, three, four, five, six, seven, eight, nine, ten, or more signals selected from 4.0±0.2 degrees two-theta, 8.1±0.2 degrees two-theta, 8.9±0.2 degrees two-theta, 9.1±0.2 degrees two-theta, 9.8±0.2 degrees two-theta, 12.1±0.2 degrees two-theta, 13.5±0.2 degrees two-theta, 14.4±0.2 degrees two-theta, 16.8±0.2 degrees two-theta, 17.9±0.2 degrees two-theta, 20.1±0.2 degrees two-theta, 20.4±0.2 degrees two-theta, 21.7±0.2 degrees two-theta, 22.0±0.2 degrees two-theta, 22.7±0.2 degrees two-theta, 26.1±0.2 degrees two-theta, 27.1±0.2 degrees two-theta, and 28.0±0.2 degrees two-theta.
198. The substantially crystalline Compound I succinic acid cocrystal hydrate according to any one of Embodiments 193-197, characterized by an X-ray powder diffractogram having signasl at 4.0±0.2 degrees two-theta, 8.1±0.2 degrees two-theta, 8.9±0.2 degrees two-theta, 9.1±0.2 degrees two-theta, 9.8±0.2 degrees two-theta, 12.1±0.2 degrees two-theta, 13.5±0.2 degrees two-theta, 14.4±0.2 degrees two-theta, 16.8±0.2 degrees two-theta, 17.9±0.2 degrees two-theta, 20.1±0.2 degrees two-theta, 20.4±0.2 degrees two-theta, 21.7±0.2 degrees two-theta, 22.0±0.2 degrees two-theta, 22.7±0.2 degrees two-theta, 26.1±0.2 degrees two-theta, 27.1±0.2 degrees two-theta, and 28.0±0.2 degrees two-theta.
199. The substantially crystalline Compound I succinic acid cocrystal (wet) according to any one of Embodiments 193-198, characterized by an X-ray powder diffractogram substantially similar to FIG. 64.
200. Substantially crystalline Compound I succinic acid cocrystal (dry) (i.e., wherein less than 15% of Compound I is in amorphous form, wherein less than 10% of Compound I is in amorphous form, wherein less than 5% of Compound I is in amorphous form).
201. The substantially crystalline Compound I succinic acid cocrystal (dry) according to Embodiment 200, wherein Compound I succinic acid cocrystal (dry) is 100% crystalline.
202. The substantially crystalline Compound I succinic acid cocrystal (dry) according to Embodiment 200 or Embodiment 201, characterized by an X-ray powder diffractogram having one, two, three, four, or five signals selected from 4.1±0.2 degrees two-theta, 20.0±0.2 degrees two-theta, 22.0±0.2 degrees two-theta, 26.1±0.2 degrees two-theta, and 27.1±0.2 degrees two-theta.
203. The substantially crystalline Compound I succinic acid cocrystal (dry) according to any one of Embodiments 200-202, characterized by an X-ray powder diffractogram having one, two, three, four, five, six, or seven signals selected from 4.1±0.2 degrees two-theta, 8.2±0.2 degrees two-theta, 20.0±0.2 degrees two-theta, 22.0±0.2 degrees two-theta, 25.5±0.2 degrees two-theta, 26.1±0.2 degrees two-theta, and 27.1±0.2 degrees two-theta.
204. The substantially crystalline Compound I succinic acid cocrystal (dry) according to any one of Embodiments 200-203, characterized by an X-ray powder diffractogram substantially similar to FIG. 65.
205. Substantially crystalline Compound I methanol solvate/hydrate (i.e., wherein less than 15% of Compound I is in amorphous form, wherein less than 10% of Compound I is in amorphous form, wherein less than 5% of Compound I is in amorphous form).
206. The substantially crystalline Compound I methanol solvate/hydrate according to Embodiment 205, wherein Compound I methanol solvate/hydrate Form C is 100% crystalline.
207. The crystalline Compound I methanol solvate/hydrate according to Embodiment 205 or Embodiment 206, characterized by an X-ray powder diffractogram having one, two, three, four, or five signals selected from 8.2±0.2 degrees two-theta, 16.4±0.2 degrees two-theta, 18.7±0.2 degrees two-theta, 20.5±0.2 degrees two-theta, and 21.5±0.2 degrees two-theta.
208. The substantially crystalline Compound I methanol solvate/hydrate according to any one of Embodiments 205-207, characterized by an X-ray powder diffractogram having one, two, three, four, five, six, seven, eight, nine, or ten signals selected from 8.2±0.2 degrees two-theta, 8.8±0.2 degrees two-theta, 10.8±0.2 degrees two-theta, 14.3±0.2 degrees two-theta, 16.4±0.2 degrees two-theta, 17.9±0.2 degrees two-theta, 18.5±0.2 degrees two-theta, 18.7±0.2 degrees two-theta, 20.5±0.2 degrees two-theta, and 21.5±0.2 degrees two-theta.
209. The substantially crystalline Compound I methanol solvate/hydrate according to any one of Embodiments 205-208, characterized by an X-ray powder diffractogram having one, two, three, four, five, six, seven, eight, nine, ten, or more signals selected from 8.2±0.2 degrees two-theta, 8.8±0.2 degrees two-theta, 10.8±0.2 degrees two-theta, 14.3±0.2 degrees two-theta, 16.4±0.2 degrees two-theta, 17.9±0.2 degrees two-theta, 18.5±0.2 degrees two-theta, 18.7±0.2 degrees two-theta, 20.0±0.2 degrees two-theta, 20.5±0.2 degrees two-theta, 21.5±0.2 degrees two-theta, and 26.9±0.2 degrees two-theta.
210. The substantially crystalline Compound I methanol solvate/hydrate according to any one of Embodiments 205-209, characterized by an X-ray powder diffractogram having signals at 8.2±0.2 degrees two-theta, 8.8±0.2 degrees two-theta, 10.8±0.2 degrees two-theta, 14.3±0.2 degrees two-theta, 16.4±0.2 degrees two-theta, 17.9±0.2 degrees two-theta, 18.5±0.2 degrees two-theta, 18.7±0.2 degrees two-theta, 20.0±0.2 degrees two-theta, 20.5±0.2 degrees two-theta, 21.5±0.2 degrees two-theta, and 26.9±0.2 degrees two-theta.
211. The substantially crystalline Compound I methanol solvate/hydrate according to any one of Embodiments 205-210, characterized by an X-ray powder diffractogram substantially similar to FIG. 67.
212. The substantially crystalline Compound I methanol solvate/hydrate according to any one of Embodiments 205-211, characterized by a ¹³C SSNMR spectrum having one, two, three, four, five, six, seven, eight, nine, ten, or more peaks selected from 163.3±0.2 ppm, 162.2±0.2 ppm, 151.6±0.2 ppm, 150.8±0.2 ppm, 138.4±0.2 ppm, 126.4±0.2 ppm, 125.4±0.2 ppm, 122.3±0.2 ppm, 121.5±0.2 ppm, 120.7±0.2 ppm, 118.9±0.2 ppm, 118.2±0.2 ppm, 117.4±0.2 ppm, 77.5±0.2 ppm, 73.2±0.2 ppm, 49.4±0.2 ppm, 36.5±0.2 ppm, 35.2±0.2 ppm, 34.1±0.2 ppm, 33.5±0.2 ppm, 32.5±0.2 ppm, 25.4±0.2 ppm, 22.8±0.2 ppm, 22.1±0.2 ppm, 21.4±0.2 ppm, 20.5±0.2 ppm, 20.0±0.2 ppm, and 19.5±0.2 ppm.
213. The substantially crystalline Compound I methanol solvate/hydrate according to any one of Embodiments 205-212, characterized by a ¹³C SSNMR spectrum having peaks at 163.3±0.2 ppm, 162.2±0.2 ppm, 151.6±0.2 ppm, 150.8±0.2 ppm, 138.4±0.2 ppm, 126.4±0.2 ppm, 125.4±0.2 ppm, 122.3±0.2 ppm, 121.5±0.2 ppm, 120.7±0.2 ppm, 118.9±0.2 ppm, 118.2±0.2 ppm, 117.4±0.2 ppm, 77.5±0.2 ppm, 73.2±0.2 ppm, 49.4±0.2 ppm, 36.5±0.2 ppm, 35.2±0.2 ppm, 34.1±0.2 ppm, 33.5±0.2 ppm, 32.5±0.2 ppm, 25.4±0.2 ppm, 22.8±0.2 ppm, 22.1±0.2 ppm, 21.4±0.2 ppm, 20.5±0.2 ppm, 20.0±0.2 ppm, and 19.5±0.2 ppm.
214. The substantially crystalline Compound I methanol solvate/hydrate according to any one of Embodiments 205-213, characterized by a ¹³C SSNMR spectrum substantially similar to FIG. 68.
215. The substantially crystalline Compound I methanol solvate/hydrate according to any one of Embodiments 205-214, characterized as having a ¹⁹F SSNMR spectrum with one, two, or three peaks selected from −64.0±0.2 ppm, −64.6±0.2 ppm, and −79.0±0.2 ppm.
216. The substantially crystalline Compound I methanol solvate/hydrate according to any one of Embodiments 205-215, characterized by a ¹⁹F SSNMR spectrum substantially similar to FIG. 69.
217. The substantially crystalline Compound I methanol solvate/hydrate according to any one of Embodiments 205-216, characterized by a monoclinic crystal system, C2 space group, and unit cell dimensions measured at 100 K on a Bruker diffractometer utilizing Cu Kₐ radiation (λ=1.54178 Å) of:

| | | | |
|---|---|---|---|
| a | 12.7 ± 0.1 Å | α | 90° |
| b | 15.7 ± 0.1 Å | β | 91.0 ± 0.2° |
| c | 43.0 ± 0.2 Å | γ | 90°. |

218. A pharmaceutical composition comprising Compound I according to any one of Embodiments 1-217, and optionally further comprising one or more additional thereapeutic agents.
219. The pharmaceutical composition according to Embodiment 218, wherein the pharmaceutical composition comprises one or more additional CFTR modulating compounds.
220. The pharmaceutical composition according to Embodiment 218 or Embodiment 219, wherein the pharmaceutical composition comprises one or more compounds selected from Compound II, Compound III, Compound III-d, Compound IV, Compound V, Compound VI, Compound VII, Compound VIII, Compound IX, Compound X, and pharmaceutically acceptable salts and deuterated derivatives thereof.
220a. The pharmaceutical composition according to any one of Embodiments 218-220, wherein the pharmaceutical composition comprises (a) one or more compounds selected from Compound II, Compound III, Compound III-d, Compound IV, Compound V, Compound VI, Compound VII, Compound VIII, Compound IX, Compound X, and pharmaceutically acceptable salts and deuterated derivatives thereof; and (b) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; U.S. Provisional Patent Application Nos. 63/328,097 and 63/393,405; Phuan, P.-W. et al. J Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J Cyst. Fibros. 2020, 19 Suppl 1, S25-S32; Crawford, D. K. J Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J. of Med. Chem. 2020, 112888.
221. The Compound I according to any one of Embodiments 1-217, or the pharmaceutical composition according to any one of Embodiments 218-220, for use in the treatment of cystic fibrosis.
222. Use of the Compound I according to any one of Embodiments 1-217, or the pharmaceutical composition according to any one of Embodiments 218-220, in the manufacture of a medicament for the treatment of cystic fibrosis.
223. A method of treating cystic fibrosis comprising administering the Compound I according to any one of Embodiments 1-217, or the pharmaceutical composition according to any one of Embodiments 218-220, to a subject in need thereof.
224. The compound or composition for use of Embodiment 221, the use of Embodiment 222, or the method of Embodiment 223, wherein the Compound I according to any one of Embodiments 1-217 or the composition according to any one of Embodiments 218-220 is administered in combination with one or more additional thereapeutic agents.
225. The compound or composition for use, the use, or the method of Embodiment 224, wherein the one or more additional thereapeutic agents comprises one or more additional CFTR modulating compounds.
226. The compound or composition for use, the use, or the method of Embodiment 224 or Embodiment 225, wherein the one or more additional thereapeutic agents comprises one or more compounds selected from Compound II, Compound III, Compound III-d, Compound IV, Compound V, Compound VI, Compound VII, Compound VIII, Compound IX, Compound X, and pharmaceutically acceptable salts and deuterated derivatives thereof.
226a. The pharmaceutical composition according to any one of Embodiments 224-226, wherein the one or more additional thereapeutic agents comprises (a) one or more compounds selected from Compound II, Compound III, Compound III-d, Compound IV, Compound V, Compound VI, Compound VII, Compound VIII, Compound IX, Compound X, and pharmaceutically acceptable salts and deuterated derivatives thereof, and (b) optionally at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; WO 2022/076618; WO 2022/076620; WO 2022/076621; WO 2022/076622; WO 2022/076624; WO 2022/076625; WO 2022/076626; WO 2022/076627; WO 2022/076628; WO 2022/076629; U.S. Provisional Patent Application Nos. 63/328,097 and 63/393,405; Phuan, P.-W. et al. J. Cyst. Fibros. 2018, 17 (5), 595-606; Pedemonte, N. et al. Sci. Adv. 2020, 6 (8), eaay9669; Phuan, P.-W. et al. Sci. Rep. 2019, 9 (1), 17640; Bose, S. et al. J Cyst. Fibros. 2020, 19 Suppl 1, S25-S32; Crawford, D. K. J Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J.-H. et al., Eur. J of Med. Chem. 2020, 112888.
227. A method of making crystalline Compound I neat Form A, comprising (i) dissolving Compound I heptane solvate Form A in methanol, (ii) adding water, (iii) stirring at room temperature for five days, (iv) collecting the solids and drying under vacuum at 40° C. for 24 hours to yield crystalline Compound I neat Form A.
228. A method of making crystalline Compound I neat Form B, comprising (i) dissolving Compound I heptane solvate Form A in dichloromethane at room temperature, and (ii) evaporating the dichloromethane slowly at room temperature to yield crystalline Compound I neat Form B.
229. A method of making crystalline Compound I hemihydrate Form C, comprising: (i) dissolving Compound I in ethanol at 25° C., (ii) adding water over 10-12 hours (ethanol to water ratio approximately 1:4 v/v), (iii) heating the slurry to 60° C. for 4 hours, (iv) cooling the slurry to 20° C. over 3 hours, (v) stirring for at least 2 hours, (vi) filtering the solids and washing with an ethanol/water solution (1:4 v/v), (vii) drying the solids in a vacuum oven at 50° C. with a slight nitrogen bleed to yield crystalline Compound I hemihydrate Form C.
230. A method of making crystalline Compound I neat Form D, comprising: (i) dissolving crystalline Compound I hemihydrate Form C in ethanol, (ii) placing the solution under nitrogen for a half hour, and (iii) placing the solution in an oven at 80° C. for ˜5 days to yield crystalline Compound I neat Form D.
231. A method of making crystalline Compound I neat Form D, comprising: (i) slurrying Compound I hemihydrate Form C in n-heptane, (ii) heating the slurry to 85° C., (iii) adding a seed of crystalline Compound I neat Form D, (iv) holding the slurry at 85±5° C., (v) cooling the slurry to 65° C. over 4 hours, (vi) collecting the solids and washing the solids with n-heptane, and (vii) drying the solids in a vacuum oven at 50° C. with a slight nitrogen bleed to yield crystalline Compound I neat Form D.
232. A method of making crystalline Compound I acetic acid solvate, comprising: (i) combining Compound I hemihydrate Form C and acetic acid, and (ii) ball milling at 7500 rpm for 2 cycles of 10 s each with a 60 s pause after each cycle, to yield crystalline Compound I acetic acid solvate.
233. A method of making crystalline Compound I heptane solvate Form B, comprising: (i) adding 1-butanol/heptane (75 v % heptane) to crystalline Compound I neat Form D and (ii) shaking the mixture at 25° C. for 2 days to yield crystalline Compound I heptane solvate Form B.
234. A method of making crystalline Compound I heptane solvate Form C, comprising: (i) adding ethyl acetate/heptane (25 v % heptane) to crystalline Compound I neat Form D and (ii) shaking at 25° C. for 2 days to yield crystalline Compound I heptane solvate Form C.
235. A method of making crystalline Compound I octane solvate, comprising shaking crystalline Compound I hemihydrate Form C in octane at 35° C. for about one week to yield crystalline Compound I octane solvate.
236. A method of making crystalline Compound I cyclohexane solvate Form A, comprising: (i) adding cyclohexane to crystalline Compound I neat Form D and (ii) shaking the mixture at 25° C. for 3 days to yield crystalline Compound I cyclohexane solvate Form A.
237. A method of making crystalline Compound I cyclohexane solvate Form B comprising: (i) adding cyclohexane to crystalline Compound I hemihydrate Form C and (ii) shaking the mixture at 80° C. for 3 days to yield crystalline Compound I cyclohexane solvate Form B.
238. A method of making crystalline Compound I cyclohexane solvate Form C comprising: (i) adding cyclohexane to crystalline Compound I hemihydrate Form C and (ii) shaking the mixture at 60° C. for one week to yield crystalline Compound I cyclohexane solvate Form C.
239. A method of making crystalline Compound I ethanol solvate comprising stirring crystalline Compound I hemihydrate Form C in ethanol at −20° C. to yield crystalline Compound I ethanol solvate.
240. A method of making crystalline Compound I solvate/hydrate (dry) comprising: (i) stirring crystalline Compound I heptane solvate Form A in water at room temperature for 2 weeks, (ii) filtering the solids, and (iii) air drying the solids to yield crystalline Compound I solvate/hydrate (dry).
241. A method of making crystalline Compound I solvate/hydrate (dry) comprising: (i) dissolving crystalline Compound I heptane solvate Form A in ethanol, (i) adding water (water/ethanol=1.23˜3.15), (iii) stirring at 60° C. for 3 days, (iv) filtering the solids, and (v) air drying the solids to yield crystalline Compound I solvate/hydrate (dry).
242. A method of making crystalline Compound I solvate/hydrate (wet) comprising: (i) adding ethanol/water 50:50 (% V/V) to crystalline Compound I hemihydrate Form C and (ii) stirring at 5° C. to yield crystalline Compound I solvate/hydrate (wet).
243. A method of making crystalline Compound I L-lysine cocrystal comprising: (i) mixing ethanol and water at ratio of 30.8% to 69.2% by volume, (ii) saturating the ethanol/water mixture with L-lysine anhydrate, (iii) saturating the mixture with crystalline Compound I hemihydrate Form C, (iv) adding crystalline Compound I hemihydrate Form C to L-lysine to make a slurry with a 1:1 molar ratio of Compound I to L-lysine, (v) mixing the slurry for 2 days, (vi) sonicating for an additional 3 hours, and (viii) isolating the solids to yield crystalline Compound I L-lysine cocrystal.
244. A method of making crystalline Compound I L-arginine cocrystal comprising: (i) preparing a 1:1 molar ratio of crystalline Compound I hemihydrate Form C and L-arginine, (ii) adding ethanol/water (30.8% to 69.2% ethanol:water by volume), (iii) ball milling the mixture at 7500 RPM for 60 seconds with 10 second pauses for 5 cycles, and (iv) drying the solids in a vacuum oven at 45° C. overnight to yield crystalline Compound I L-arginine cocrystal.
245. A method of making crystalline Compound I L-phenylalanine cocrystal comprising: (i) preparing a 1:1 molar ratio of crystalline Compound I hemihydrate Form C and L-phenylalanine, (ii) adding ethanol/water (30.8% to 69.2% ethanol:water by volume), (iii) ball milling the mixture at 7500 RPM for 60 seconds with 10 second pauses for 5 cycles, and (iv) drying the solids in a vacuum oven at 45° C. overnight to yield crystalline Compound I L-phenylalanine cocrystal.
246. A method of making crystalline Compound I succinic acid cocrystal (wet) comprising: (i) preparing a 1:1 molar ratio of crystalline Compound I hemihydrate Form C and succinic acid, (ii) adding ethanol/water (30.8% to 69.2% ethanol:water by volume), (iii) ball milling the mixture at 7500 RPM for 60 seconds with 10 second pauses for 5 cycles, (iv) drying the solids in a vacuum oven at 45° C. overnight, and (v) placing the solids in a humidity chamber at 40° C., 75% Relative Humidity to yield crystalline Compound I succinic acid cocrystal hydrate.
247. A method of making crystalline Compound I succinic acid cocrystal (dry) comprising: (i) preparing a 1:1 molar ratio of crystalline Compound I hemihydrate Form C and succinic acid, (ii) adding ethanol/water (30.8% to 69.2% ethanol:water by volume), (iii) ball milling the mixture at 7500 RPM for 60 seconds with 10 second pauses for 5 cycles, and (iv) drying the solids in a vacuum oven at 45° C. overnight to yield crystalline Compound I succinic acid cocrystal.
248. A method of making crystalline Compound I methanol solvate/hydrate comprising: (i) combining crystalline Compound I hemihydrate Form C and methanol, (ii) stirring the mixture, and (iii) isolating the solids to yield crystalline Compound I methanol solvate/hydrate.
249. A method of making Compound I neat amorphous form comprising: (i) dissolving tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate in ethanol, (ii) adding 10% Pd/C, (iii) stirring at room temperature under hydrogen, (iv) isolating and evaporating the liquid phase, (v) redissolving in dichloromethane, (vi) cooling the solution in an ice bath and treating with trifluoroacetic acid, (viii) stirring at room temperature for 2 h, (ix) diluting the solution with heptane, evaporating, and drying to yield a solid, (x) dissolving the solid in dichloromethane and diluting with heptane, (xi) stirring the suspension at room temperature, (xii) filtering off the solids, (xiii) concentrating the mother liquor and purifying the resulting solid by reverse phase chromatography to yield Compound I neat amorphous form.

### B. Set 2

1. A method of preparing Compound I:

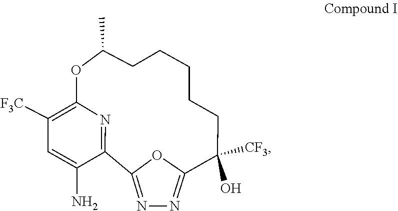 [SMILES: C[C@@H]1CCCCC[C@](O)(C(F)(F)F)C2=NN=C(O2)c2nc(c(C(F)(F)F)cc2N)O1]

or a stereoisomer of Compound I, or a deuterated derivative of Compound I or a stereoisomer thereof, or pharmaceutically acceptable salts of any of the foregoing, wherein the method comprises converting a compound of Formula (1):

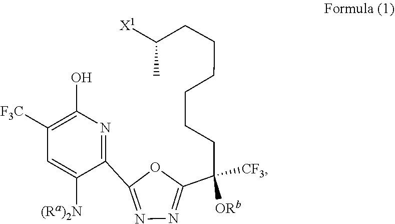

or a stereoisomer thereof, or a deuterated derivative of the compound of Formula (1) or its stereoisomer, or a salt of any of the foregoing, into Compound I, or a stereoisomer thereof or a deuterated derivative of Compound I or its stereoisomer, or pharmaceutically acceptable salt of any of the foregoing, wherein:
   X¹ is selected from OH, OTs, OMs, ONs, and OTf;
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
   R^{b} is selected from benzyl (Bn), naphthylmethyl (Nap), biphenylmethyl, Ac, TFA, and Bz.
1a. A method of preparing Compound I:

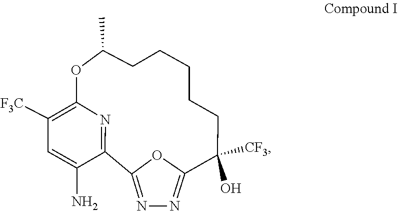 [SMILES: C[C@@H]1CCCCC[C@](O)(C(F)(F)F)C2=NN=C(O2)c2nc(c(C(F)(F)F)cc2N)O1]

or a stereoisomer of Compound I, or a deuterated derivative of Compound I or a stereoisomer thereof, or pharmaceutically acceptable salts of any of the foregoing, wherein the method comprises converting a compound of Formula (1a):

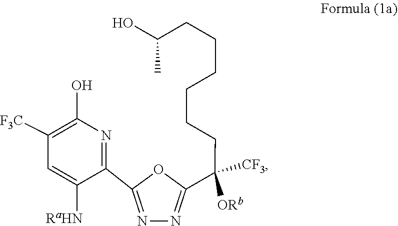

or a stereoisomer thereof, or a deuterated derivative of the compound of Formula (1a) or its stereoisomer, or a salt of any of the foregoing, into Compound I, or a stereoisomer thereof or a deuterated derivative of Compound I or its stereoisomer, or pharmaceutically acceptable salt of any of the foregoing, wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
2. The method according to Embodiment 1, wherein N(R^{a})₂ is NHBoc and R^{b} is Bn.
2a. The method according to Embodiment 1, wherein X¹ is OTs
2b. The method according to Embodiment 1a, wherein R^{a} is Boc and R^{b} is Bn.
3. The method according to Embodiment 1, wherein the method comprises converting a compound of Formula (2),

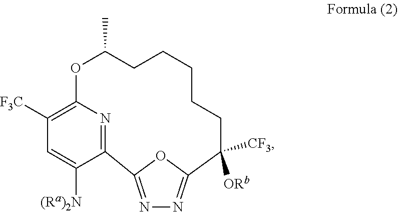

or a stereoisomer thereof, or a deuterated derivative of the compound of Formula (2) or its stereoisomer, or a salt of any of the foregoing, into Compound I, or a stereoisomer thereof, or a deuterated derivative of Compound I or its stereoisomer, or a pharmaceutically acceptable salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
   R^{b} is selected from benzyl (Bn), naphthylmethyl (Nap), biphenylmethyl, Ac, TFA, and Bz.
3a. The method according to Embodiment 1, wherein the method comprises converting a compound of Formula (2a),

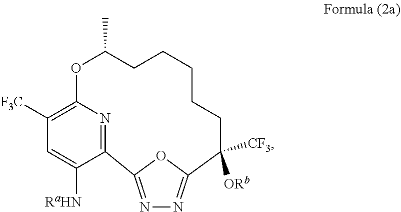

or a stereoisomer thereof, or a deuterated derivative of the compound of Formula (2a) or its stereoisomer, or a salt of any of the foregoing, into Compound I, or a stereoisomer thereof, or a deuterated derivative of Compound I or its stereoisomer, or a pharmaceutically acceptable salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
4. The method according to Embodiment 3, wherein N(R^{a})₂ is NHBoc and R^{b} is Bn.
4a. The method according to Embodiment 3a, wherein R^{a} is Boc and R^{b} is Bn.
5. The method according to Embodiment 1 or 3, wherein the method comprises converting a compound of Formula (3):

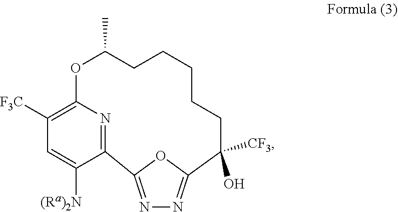

or a stereoisomer thereof, or a deuterated derivative of the compound of Formula (3) or its stereoisomer, or a salt of any of the foregoing, into Compound I, or a stereoisomer thereof, or a deuterated derivative of Compound I or its stereoisomer, or a pharmaceutically acceptable salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).
5a. The method according to Embodiment 1a or 3a, wherein the method comprises converting a compound of Formula (3a):

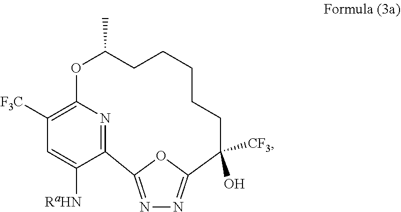

or a stereoisomer thereof, or a deuterated derivative of the compound of Formula (3a) or its stereoisomer, or a salt of any of the foregoing, into Compound I, or a stereoisomer thereof, or a deuterated derivative of Compound I or its stereoisomer, or a pharmaceutically acceptable salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).
6. The method according to Embodiment 5, wherein N(R^{a})₂ is NHBoc.
6a. The method according to Embodiment 5a, wherein R^{a} is Boc.
7. The method according to Embodiment 6 or 6a, wherein converting the compound of Formula (3), or a stereoisomer thereof, or a deuterated derivative of the compound of Formula (3) or its stereoisomer, or a salt of any of the foregoing, into Compound I, or a stereoisomer thereof, or a deuterated derivative of Compound I or its stereoisomer, or a pharmaceutically acceptable salt of any of the foregoing, or wherein converting the compound of Formula (3a), or a stereoisomer thereof, or a deuterated derivative of the compound of Formula (3a) or its stereoisomer, or a salt of any of the foregoing, into Compound I, or a stereoisomer thereof, or a deuterated derivative of Compound I or its stereoisomer, or a pharmaceutically acceptable salt of any of the foregoing, is performed in the presence of an acid.
8. The method according to Embodiment 7, wherein the acid is selected from trifluoroacetic acid (TFA), hydrochloric acid (HCl), methanesulfonic acid (MsOH), phosphoric acid (H₃PO₄), and sulfuric acid (H₂SO₄).
9. The method according to Embodiment 7 or 8, wherein the acid is trifluoroacetic acid (TFA).
10. The method according to Embodiment 5, wherein the compound of Formula (3):

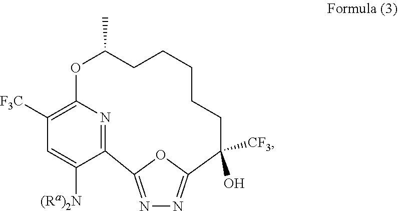

or a stereoisomer thereof, or a deuterated derivative of the compound of Formula (3) or its stereoisomer, or a salt of any of the foregoing, is prepared by converting a compound of Formula (2):

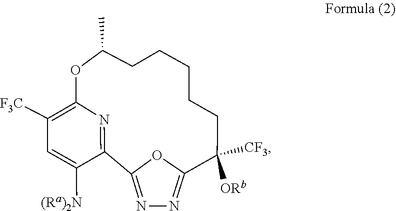

or a stereoisomer thereof, or a deuterated derivative of the compound of Formula (2) or its stereoisomer, or a salt of any of the foregoing, into the compound of Formula (3), or a stereoisomer thereof, or a deuterated derivative of the compound of Formula (3) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
   R^{b} is selected from benzyl (Bn), naphthylmethyl (Nap), biphenylmethyl, Ac, TFA, and Bz.
10a. The method according to Embodiment 5a, wherein the compound of Formula (3a):

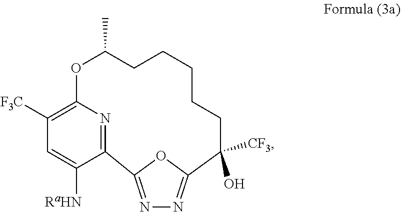

or a stereoisomer thereof, or a deuterated derivative of the compound of Formula (3a) or its stereoisomer, or a salt of any of the foregoing, is prepared by converting a compound of Formula (2a):

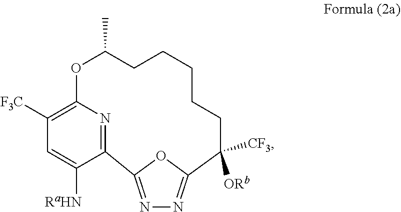

or a stereoisomer thereof, or a deuterated derivative of the compound of Formula (2a) or its stereoisomer, or a salt of any of the foregoing, into the compound of Formula (3a), or a stereoisomer thereof, or a deuterated derivative of the compound of Formula (3a) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz
11. The method according to Embodiment 10, wherein R^{a} is Boc and R^{b} is Bn.
11a. The method according to Embodiment 10a, wherein R^{a} is Boc.
12. The method according to Embodiment 11 or 11a, wherein converting the compound of Formula (2), or a stereoisomer thereof, or a deuterated derivative of the compound of Formula (2) or its stereoisomer, or a salt of any of the foregoing, into the compound of Formula (3), or a stereoisomer thereof, or a deuterated derivative of the compound of Formula (3) or its stereoisomer, or a salt of any of the foregoing; or wherein converting the compound of Formula (2a), or a stereoisomer thereof, or a deuterated derivative of the compound of Formula (2a) or its stereoisomer, or a salt of any of the foregoing, into the compound of Formula (3a), or a stereoisomer thereof, or a deuterated derivative of the compound of Formula (3a) or its stereoisomer, or a salt of any of the foregoing, is performed in the presence of hydrogen gas (H₂) and a palladium catalyst.
13. The method according to Embodiment 12, wherein the palladium catalyst is selected from palladium on carbon (Pd/C) and palladium on alumina (Pd/Al).
14. The method according to Embodiment 12 or 13, wherein the palladium catalyst is palladium on carbon (Pd/C).
15. The method according to Embodiment 3 or 10, wherein the compound of Formula (2):

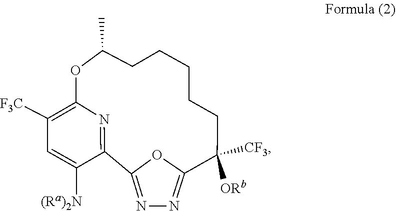

or a stereoisomer thereof, or a deuterated derivative of the compound of Formula (2) or its stereoisomer, or a salt of any of the foregoing is prepared by converting a compound of Formula (1):

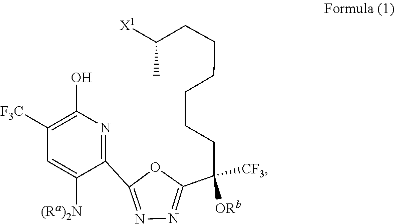

or a stereoisomer thereof, or a deuterated derivative of the compound of Formula (1) or its stereoisomer, or a salt of any of the foregoing, into the compound of Formula (2), or a stereoisomer thereof, or a deuterated derivative of the compound of Formula (2) or its stereoisomer, or a salt of any of the foregoing,
wherein:
   X¹ is selected from OTs, OMs, ONs, and OTf;
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
   R^{b} is selected from benzyl (Bn), naphthylmethyl (Nap), biphenylmethyl, Ac, TFA, and Bz.
15a. The method according to Embodiment 3a or 10a, wherein the compound of Formula (2a):

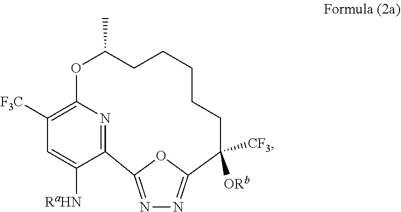

or a stereoisomer thereof, or a deuterated derivative of the compound of Formula (2a) or its stereoisomer, or a salt of any of the foregoing is prepared by converting a compound of Formula (1a):

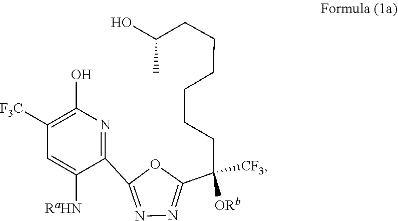

or a stereoisomer thereof, or a deuterated derivative of the compound of Formula (1a) or its stereoisomer, or a salt of any of the foregoing, into the compound of Formula (2a), or a stereoisomer thereof, or a deuterated derivative of the compound of Formula (2a) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
16. The method according to Embodiment 15, wherein N(R^{a})₂ is NHBoc and R^{b} is Bn.
16a. The method according to Embodiment 10a, wherein R^{a} is Boc and R^{b} is Bn.
16b. The method according to Embodiment 10a, wherein X¹ is OTs.
16c. The method according to any one of Embodiments 15 to 16a, wherein converting the compound of Formula (1), or a stereoisomer thereof, or a deuterated derivative of the compound of Formula (1) or its stereoisomer, or a salt of any of the foregoing, into the compound of Formula (2), or a stereoisomer thereof, or a deuterated derivative of the compound of Formula (2) or its stereoisomer, or a salt of any of the foregoing, is performed in the presence of Bu₄NOAc and NaHCO₃.
17. The method according to Embodiment 15a or 16a, wherein converting the compound of Formula (1a), or a stereoisomer thereof, or a deuterated derivative of the compound of Formula (1a) or its stereoisomer, or a salt of any of the foregoing, into the compound of Formula (2a), or a stereoisomer thereof, or a deuterated derivative of the compound of Formula (2a) or its stereoisomer, or a salt of any of the foregoing, is performed in the presence of a diazodicarboxylate and a phosphine.
18. The method according to Embodiment 17, wherein the diazodicarboxylate is selected from diisopropyl azodicarboxylate (DIAD), di-2-methoxyethyl azodicarboxylate (DMEAD), di-tert-butyl azodicarboxylate (DTBAD), di-(4-chlorobenzyl)azodicarboxylate (DCAD), 4,4′-azopyridine (AZPY) and 1,1′-(azodicarbonyl)dipiperidine (ADDP).
19. The method according to Embodiment 17 or 18, wherein the phosphine is selected from triphenylphosphine (PPh₃), tris(4-methoxyphenyl)phosphine (P(4-OMe-Ph)₃), tris(4-chlorophenyl)phosphine (P(4-Cl-Ph)₃), tricyclohexylphosphine (PCy₃), methyldiphenylphosphine (PPh₂Me), diphenyl-2-pyridylphosphine (P(2-pyridyl)Ph₂), dicyclohexylphenylphosphine (PCy₂Ph), and 1,2-bis(diphenylphosphino)ethane (dppe).
20. The method according to any one of Embodiments 17 to 19, wherein the diazodicarboxylate is diisopropyl azodicarboxylate (DIAD) and the phosphine is triphenylphosphine (PPh₃).
21. The method according to Embodiment 1 or 15, wherein the compound of Formula (1):

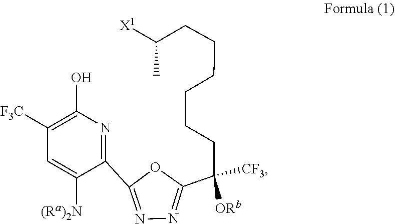

or a stereoisomer thereof, or a deuterated derivative of the compound of Formula (1) or its stereoisomer, or a salt of any of the foregoing, is prepared by converting a compound of Formula (4):

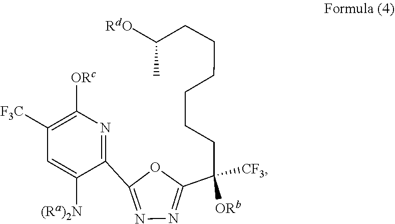

or a stereoisomer thereof, or a deuterated derivative of the compound of Formula (4) or its stereoisomer, or a salt of any of the foregoing, into the compound of Formula (1), or a stereoisomer thereof, or a deuterated derivative of the compound of Formula (1) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂;
   R^{b} is selected from benzyl (Bn), naphthylmethyl (Nap), biphenylmethyl, Ac, TFA, and Bz;
   R^{c} is selected from Bn, Me, and allyl; and
   R^{d} is selected from Ts, Ms, Ns, and Tf.
21a. The method according to Embodiment 1a or 15a, wherein the compound of Formula (1a):

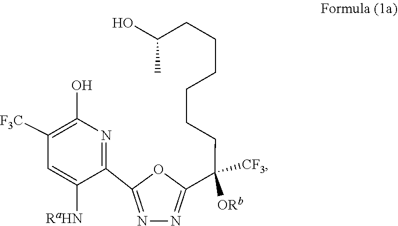

or a stereoisomer thereof, or a deuterated derivative of the compound of Formula (1) or its stereoisomer, or a salt of any of the foregoing, is prepared by converting a compound of Formula (4):

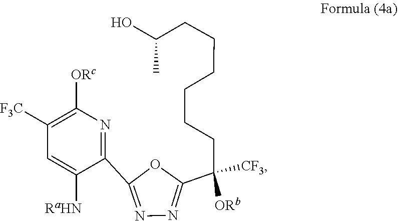

or a stereoisomer thereof, or a deuterated derivative of the compound of Formula (4a) or its stereoisomer, or a salt of any of the foregoing, into the compound of Formula (1a), or a stereoisomer thereof, or a deuterated derivative of the compound of Formula (1a) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{b} is selected from benzyl (Bn), naphthylmethyl (Nap), biphenylmethyl, Ac, TFA, and Bz; and
   R^{c} is selected from Bn, Me, and allyl.
22. The method according to Embodiment 21, wherein N(R^{a})₂ is NHBoc, R^{b} is Bn, R^{c} is allyl, and R^{d} is Ts.
22a. The method according to Embodiment 21a, wherein R^{a} is Boc, R^{b} is Bn, and R^{c} is Bn.
23. The method according to Embodiment 22, wherein converting the compound of Formula (4), or a stereoisomer thereof, or a deuterated derivative of the compound of Formula (4) or its stereoisomer, or a salt of any of the foregoing, into the compound of Formula (1), or a stereoisomer thereof, or a deuterated derivative of the compound of Formula (1) or its stereoisomer, or a salt of any of the foregoing, is performed in the presence of a palladium catalyst.
23a. The method according to Embodiment 22a, wherein converting the compound of Formula (4a), or a stereoisomer thereof, or a deuterated derivative of the compound of Formula (4a) or its stereoisomer, or a salt of any of the foregoing, into the compound of Formula (1a), or a stereoisomer thereof, or a deuterated derivative of the compound of Formula (1a) or its stereoisomer, or a salt of any of the foregoing, is performed in the presence of hydrogen gas (H₂) and a palladium catalyst.
24. The method according to Embodiment 23, wherein converting the compound of Formula (4), or a stereoisomer thereof, or a deuterated derivative of the compound of Formula (4) or its stereoisomer, or a salt of any of the foregoing, into the compound of Formula (1), or a stereoisomer thereof, or a deuterated derivative of the compound of Formula (1) or its stereoisomer, or a salt of any of the foregoing, is performed in the presence of Pd(PPh₃)₄.
24a. The method according to Embodiment 23a, wherein the palladium catalyst is selected from palladium on carbon (Pd/C) and palladium on alumina (Pd/Al).
25. The method according to Embodiment 23, 23a, or 24a, wherein the palladium catalyst is palladium on carbon (Pd/C).
26. The method according to Embodiment 21, wherein the compound of Formula (4):

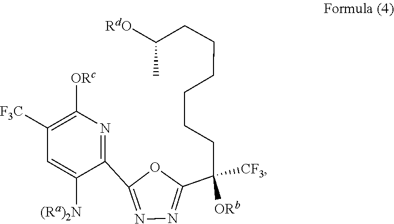

or a stereoisomer thereof, or a deuterated derivative of the compound of Formula (4) or its stereoisomer, or a salt of any of the foregoing, is prepared by converting a compound of Formula (5):

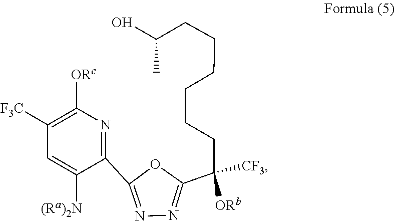

or a stereoisomer thereof, or a deuterated derivative of the compound of Formula (5) or its stereoisomer, or a salt of any of the foregoing, into the compound of Formula (4), or a stereoisomer thereof, or a deuterated derivative of the compound of Formula (4) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); or N(R^{a})₂ is NO₂;
   R^{b} is selected from benzyl (Bn), naphthylmethyl (Nap), biphenylmethyl, Ac, TFA, and Bz;
   R^{c} is selected from Bn, Me, and allyl; and
   R^{d} is selected from Ts, Ms, Ns, and Tf.
26a. The method according to Embodiment 26, wherein N(R^{a})₂ is NHBoc, R^{b} is Bn, R^{c} is allyl, and R^{d} is Ts.
26b. The method according to Embodiment 26, wherein converting the compound of Formula (5), or a stereoisomer thereof, or a deuterated derivative of the compound of Formula (5) or its stereoisomer, or a salt of any of the foregoing, into the compound of Formula (4), or a stereoisomer thereof, or a deuterated derivative of the compound of Formula (4) or its stereoisomer, or a salt of any of the foregoing, is performed in the presence of sulfonyl chloride, an amine base, and optionally in the presence of an additive.
26c. The method according to Embodiment 26b, wherein sulfonyl chloride is p-toluenesulfonyl chloride (TsCl).
26d. The method according to Embodiment 26b or 26c, wherein the amine base is N,N-diisopropylethylamine (DIPEA).
26e. The method according to any one of Embodiments 26b to 26d, wherein the additive is
   1,4-diazabicyclo[2.2.2]octane (DABCO)
26f. The method according to Embodiment 21a, wherein the compound of Formula (4a):

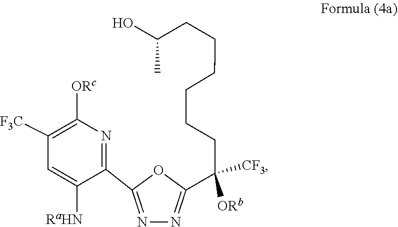

or a stereoisomer thereof, or a deuterated derivative of the compound of Formula (4a) or its stereoisomer, or a salt of any of the foregoing, is prepared by converting a compound of Formula (5a):

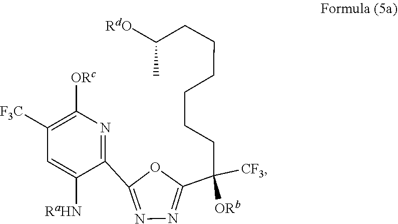

or a stereoisomer thereof, or a deuterated derivative of the compound of Formula (5a) or its stereoisomer, or a salt of any of the foregoing, into the compound of Formula (4a), or a stereoisomer thereof, or a deuterated derivative of the compound of Formula (4a) or its stereoisomer, or a salt of any of the foregoing, wherein:
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{b} is selected from benzyl (Bn), naphthylmethyl (Nap), biphenylmethyl, Ac, TFA, and Bz;
   R^{c} is selected from Bn, Me, and allyl; and
   R^{d} is selected from H, Bn, TBS, and TBDPS 27. The method according to Embodiment 26f, wherein R^{a} is Boc, R^{b} is Bn, R^{c} is Bn, and R^{d} is TBDPS.
28. The method according to Embodiment 27, wherein converting the compound of Formula (5a), or a stereoisomer thereof, or a deuterated derivative of the compound of Formula (5a) or its stereoisomer, or a salt of any of the foregoing, into the compound of Formula (4a), or a stereoisomer thereof, or a deuterated derivative of the compound of Formula (4a) or its stereoisomer, or a salt of any of the foregoing, is performed in the presence of tetrabutylammonium fluoride (TBAF), trifluoroacetic acid (TFA), sulfuric acid, and/or hydrochloric acid (HCl).
29. The method according to Embodiment 27 or 28, wherein converting the compound of Formula (5a), or a stereoisomer thereof, or a deuterated derivative of the compound of Formula (5a) or its stereoisomer, or a salt of any of the foregoing, is performed in the presence of tetrabutylammonium fluoride (TBAF).
30. The method according to Embodiment 26, wherein the compound of Formula (5):

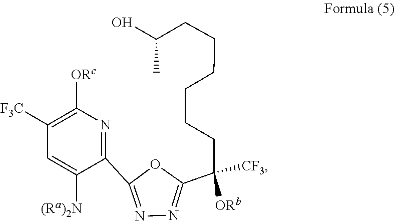

or a stereoisomer thereof, or a deuterated derivative of the compound of Formula (5) or its stereoisomer, or a salt of any of the foregoing, is prepared by converting a compound of Formula (6):

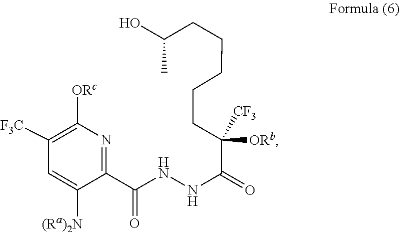

or a stereoisomer thereof, or a deuterated derivative of the compound of Formula (6) or its stereoisomer, or a salt of any of the foregoing, into the compound of Formula (5), or a stereoisomer thereof, or a deuterated derivative of the compound of Formula (5) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); or N(R^{a})₂ is NO₂;
   R^{b} is selected from benzyl (Bn), naphthylmethyl (Nap), biphenylmethyl, Ac, TFA, and Bz; and
   R^{c} is selected from Bn, Me, and allyl.
30a. The method according to Embodiment 26f, wherein the compound of Formula (5a):

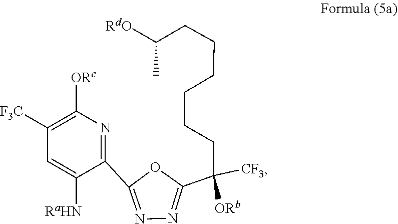

or a stereoisomer thereof, or a deuterated derivative of the compound of Formula (5) or its stereoisomer, or a salt of any of the foregoing, is prepared by converting a compound of Formula (6a):

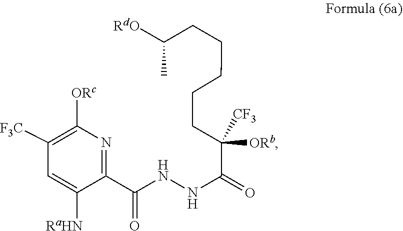

or a stereoisomer thereof, or a deuterated derivative of the compound of Formula (6a) or its stereoisomer, or a salt of any of the foregoing, into the compound of Formula (5a), or a stereoisomer thereof, or a deuterated derivative of the compound of Formula (5a) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{b} is selected from benzyl (Bn), naphthylmethyl (Nap), biphenylmethyl, Ac, TFA, and Bz;
   R^{c} is selected from Bn, Me, and allyl; and
   R^{d} is selected from H, Bn, TBS, and TBDPS.
31. The method according to Embodiment 30, wherein N(R^{a})₂ is NHBoc, R^{b} is Bn, and R^{c} is allyl.
31a. The method according to Embodiment 30a, wherein R^{a} is Boc, R^{b} is Bn, R^{c} is Bn, and R^{d} is TBDPS.
32. The method according to any one of Embodiments 30 to 31a, wherein converting the compound of Formula (6), or a stereoisomer thereof, or a deuterated derivative of the compound of Formula (6) or its stereoisomer, or a salt of any of the foregoing, into the compound of Formula (5), or a stereoisomer thereof, or a deuterated derivative of the compound of Formula (5) or its stereoisomer, or a salt of any of the foregoing, is performed in the presence of a sulfonyl chloride, an amine base, and optionally in the presence of a silyl chloride and/or an additive, or wherein converting the compound of Formula (6a), or a stereoisomer thereof, or a deuterated derivative of the compound of Formula (6a) or its stereoisomer, or a salt of any of the foregoing, into the compound of Formula (5a), or a stereoisomer thereof, or a deuterated derivative of the compound of Formula (5a) or its stereoisomer, or a salt of any of the foregoing, is performed in the presence of a sulfonyl chloride, an amine base, and optionally in the presence of an additive.
33. The method according to Embodiment 32, wherein the sulfonyl chloride is p-toluenesulfonyl chloride (TsCl).
34. The method according to Embodiment 32 or 33, wherein the amine base is N,N-diisopropylethylamine (DIPEA).
35. The method according to any one of Embodiments 32 to 34, wherein the additive is
   1,4-diazabicyclo[2.2.2]octane (DABCO).
36. The method according to any one of Embodiments 32 to 35, wherein the sulfonyl chloride is p-toluenesulfonyl chloride (TsCl), the amine base is N,N-diisopropylethylamine (DIPEA), and the additive is 1,4-diazabicyclo[2.2.2]octane (DABCO).
36a. The method according to to any one of Embodiments 32 to 36, wherein the silyl chloride is TMSCl.
37. The method according to Embodiment 30 or 30a, wherein the compound of Formula (6) or Formula (6a):

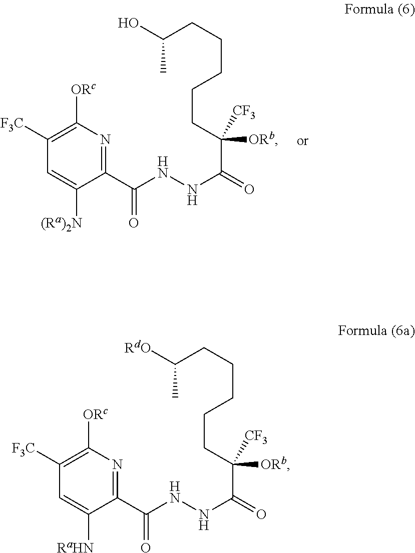

or a stereoisomer thereof, or a deuterated derivative of the compound of Formula (6) or Formula (6a) or its stereoisomer, or a salt of any of the foregoing, is prepared by combining a compound of Formula (7):

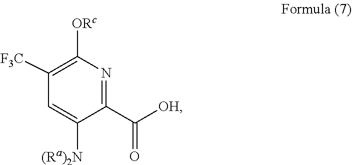

or a deuterated derivative or salt thereof, with a compound of Formula (8):

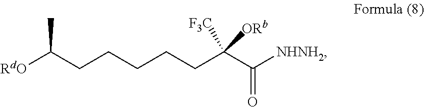

Or a stereoisomer thereof, or a deuterated derivative of the compound of Formula (8) or its stereoisomer, or a salt of any of the foregoing, to produce the compound of Formula (6) or Formula (6a), or a stereoisomer thereof, or a deuterated derivative of the compound of Formula (6) or Formula (6a), or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); or N(R^{a})₂ is NO₂;
   R^{b} is selected from benzyl (Bn), naphthylmethyl (Nap), biphenylmethyl, Ac, TFA, and Bz;
   R^{c} is selected from Bn, Me, and allyl; and
   R^{d} is selected from H, Bn, TBS, and TBDPS.
37a. The method according to Embodiment 37, wherein R^{d} is H.
37b. The method according to Embodiment 30, wherein the compound of Formula (6a):

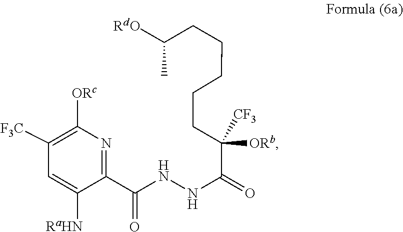

or a stereoisomer thereof, or a deuterated derivative of the compound of Formula (6) or its stereoisomer, or a salt of any of the foregoing, is prepared by combining a compound of Formula (7a):

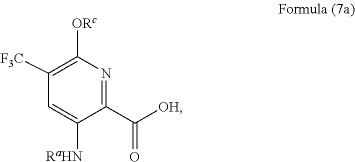

or a deuterated derivative or salt thereof, with a compound of Formula (8):

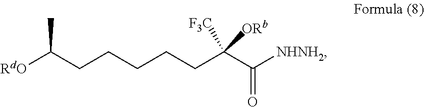

Or a stereoisomer thereof, or a deuterated derivative of the compound of Formula (8) or its stereoisomer, or a salt of any of the foregoing, to produce the compound of Formula (6a), or a stereoisomer thereof, or a deuterated derivative of the compound of Formula (6a), or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{b} is selected from benzyl (Bn), naphthylmethyl (Nap), biphenylmethyl, Ac, TFA, and Bz;
   R^{c} is selected from Bn, Me, and allyl; and
   R^{d} is selected from H, Bn, TBS, and TBDPS.
38. The method according to Embodiment 37 or 37b, wherein R^{a} is Boc, R^{b} is Bn, R^{c} is Bn, and R^{d} is TBDPS.
39. The method according to any one of Embodiments 37 to 38, wherein combining the compound of Formula (7) or Formula (7a), or a deuterated derivative thereof, or a salt of any of the foregoing, with the compound of Formula (8), or a stereoisomer thereof, or a deuterated derivative of the compound of Formula (8) or its stereoisomer, or a salt of any of the foregoing, is performed in the presence of a peptide coupling agent and an amine base.
40. The method according to Embodiment 39, wherein the peptide coupling agent is 2-chloro-4,6-dimethoxy-1,3,5-triazine (CDMT).
41. The method according to Embodiment 39 or 40, wherein the amine base is N-methylmorpholine (NMM).
42. The method according to any one of Embodiments 39 to 41, wherein the peptide coupling agent is 2-chloro-4,6-dimethoxy-1,3,5-triazine (CDMT) and the amine base is N-methylmorpholine (NMM).
43. The method according to Embodiment 37, wherein the compound of Formula (7):

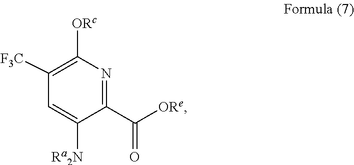

or a deuterated derivative or salt thereof, is prepared by a method comprising converting a compound of Formula (9):

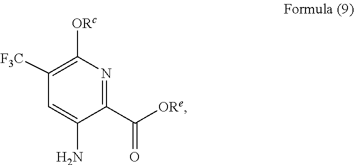

or a deuterated derivative or salt thereof, into a compound of Formula (7):
or a deuterated derivative thereof, or a salt of any of the foregoing;
wherein:
   each R^{a} is independently selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{c} is selected from Bn, Me, and allyl; and
   R^{e} is selected from Me, Et, n-Pr, i-Pr, and t-Bu.
43a. The method according to Embodiment 37a, wherein the compound of Formula (7a):

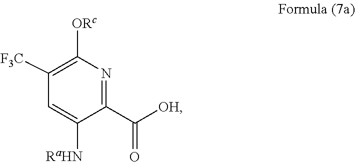

or a deuterated derivative or salt thereof, is prepared by a method comprising the following steps:
a) converting a compound of Formula (9a):

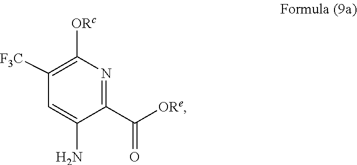

or a deuterated derivative or salt thereof, into a compound of Formula (10a):

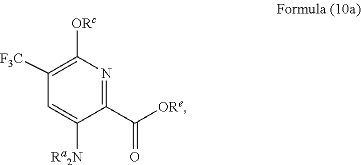

or a deuterated derivative thereof, or a salt of any of the foregoing; and
   b) converting the compound of Formula (10a), or a deuterated derivative thereof, or a salt of any of the foregoing, into the compound of Formula (7a), or a deuterated derivative or salt thereof,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{c} is selected from Bn, Me, and allyl; and
   R^{e} is selected from Me, Et, n-Pr, i-Pr, and t-Bu.
44. The method according to Embodiment 43, wherein NR^{a}₂ is NHBoc, R^{c} is Bn, and R^{e} is Me.
44a. The method according to Embodiment 43a, wherein R^{a} is Boc, R^{c} is Bn, and R^{e} is Me.
45. The method according to Embodiment 43 or 44, wherein step a) is performed in the presence of di-tert-butyl dicarbonate (Boc₂O).
46. The method according to any one of Embodiments 43 to 45, wherein step b) is performed in the presence of an aqueous hydroxide base.
47. The method according to Embodiment 46, wherein the aqueous hydroxide base is selected from aqueous lithium hydroxide (LiOH), aqueous sodium hydroxide (NaOH), and aqueous potassium hydroxide (KOH).
48. The method according to Embodiment 46 or 47, wherein the aqueous hydroxide base is aqueous lithium hydroxide (LiOH).
49. The method according to Embodiment 43, wherein the compound of Formula (9):

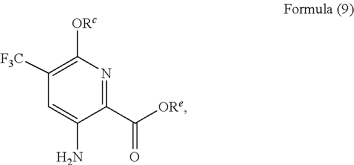

or a deuterated derivative or salt thereof, is prepared by converting a compound of Formula (11):

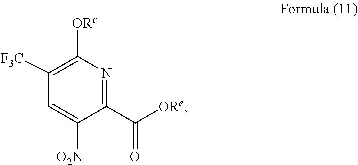

or a deuterated derivative or salt thereof, into the compound of Formula (9), or a deuterated derivative or salt thereof,
wherein:
   R^{c} is selected from Bn, Me, and allyl; and
   R^{e} is selected from Me, Et, n-Pr, i-Pr, and t-Bu.
50. The method according to Embodiment 49, wherein R^{c} is Bn and and R^{e} is Me.
51. The method according to Embodiment 49 or 50, wherein converting the compound of Formula (11), or a deuterated derivative or salt thereof, into the compound of Formula (9), or a deuterated derivative or salt thereof, is performed in the presence of reducing conditions.
52. The method according to Embodiment 51, wherein the reducing conditions are selected from:
   (i) aqueous sodium dithionite (Na₂S₂O₄) and
   (ii) iron (Fe) and acetic acid (AcOH).
53. The method according to Embodiment 49, wherein the compound of Formula (11):

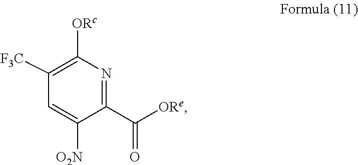

or a deuterated derivative or salt thereof, is prepared by converting a compound of Formula (12):

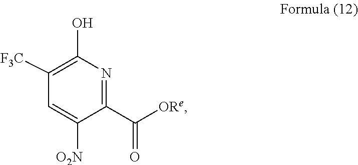

or a deuterated derivative or salt thereof, into the compound of Formula (11), or a deuterated derivative or salt thereof,
wherein:
   R^{c} is selected from Bn, Me, and allyl; and
   R^{e} is selected from Me, Et, n-Pr, i-Pr, and t-Bu.
54. The method according to Embodiment 53, wherein R^{c} is Bn and R^{e} is Me.
55. The method according to Embodiment 53 or 54, wherein converting the compound of Formula (12), or a deuterated derivative or salt thereof, into the compound of Formula (11), or a deuterated derivative or salt thereof, is performed in the presence of R^{c}OH, a diazodicarboxylate, and a phosphine.
56. The method according to Embodiment 55, wherein R^{c}OH is benzyl alcohol (BnOH).
57. The method according to Embodiment 55 or 56, wherein the diazodicarboxylate is selected from diisopropyl azodicarboxylate (DIAD), di-2-methoxyethyl azodicarboxylate (DMEAD), di-tert-butyl azodicarboxylate (DTBAD), di-(4-chlorobenzyl)azodicarboxylate (DCAD), 4,4′-azopyridine (AZPY) and 1,1′-(azodicarbonyl)dipiperidine (ADDP).
58. The method according to any one of Embodiments 55 to 57, wherein the phosphine is selected from triphenylphosphine (PPh₃), tris(4-methoxyphenyl)phosphine (P(4-OMe-Ph)₃), tris(4-chlorophenyl)phosphine (P(4-Cl-Ph)₃), tricyclohexylphosphine (PCy₃), methyldiphenylphosphine (PPh₂Me), diphenyl-2-pyridylphosphine (P(2-pyridyl)Ph₂), dicyclohexylphenylphosphine (PCy₂Ph), and 1,2-bis(diphenylphosphino)ethane (dppe).
59. The method according to any one of Embodiments 55 to 58, wherein R^{c}OH is benzyl alcohol (BnOH), the diazodicarboxylate is diisopropyl azodicarboxylate (DIAD), and the phosphine is triphenyl phosphine (PPh₃).
60. The method according to Embodiment 37 or 37b, wherein the compound of Formula (8):

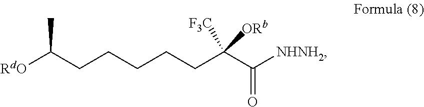

or a stereoisomer thereof, or a deuterated derivative of the compound of Formula (8) or its stereoisomer, or a salt of any of the foregoing, is prepared by converting a compound of Formula (13):

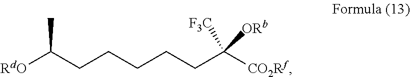

or a stereoisomer thereof, or a deuterated derivative of the compound of Formula (13) or its stereoisomer, or a salt of any of the foregoing, into the compound of Formula (8), or a stereoisomer thereof, or a deuterated derivative of the compound of Formula (8) or its stereoisomer, or a salt of any of the foregoing,
wherein:
   R^{b} is selected from benzyl (Bn), naphthylmethyl (Nap), biphenylmethyl, Ac, TFA, and Bz;
   R^{d} is selected from H, Bn, TBS, and TBDPS; and
   R^{f} is selected from Me, Et, and Bn.
61. The method according to Embodiment 60, wherein R^{b} is Bn, R^{d} is TBDPS, and R^{f} is Et.
62. The method according to Embodiment 61, wherein converting the compound of Formula (13), or a stereoisomer thereof, or a deuterated derivative of the compound of Formula (13) or its stereoisomer, or a salt of any of the foregoing, into the compound of Formula (8), or a stereoisomer thereof, or a deuterated derivative of the compound of Formula (8) or its stereoisomer, or a salt of any of the foregoing, is performed in the presence of a hydrazine source and optionally an additive.
63. The method according to Embodiment 62, wherein the hydrazine source is selected from hydrazine hydrate, hydrazine monohydrochloride, hydrazine dihydrochloride, and hydrazine sulfate salt.
64. The method according to Embodiment 62 or 63, wherein the additive is selected from guanidine bases.
65. The method according to any one of Embodiments 62 to 64, wherein the additive is 1,5,7-triazabicyclo[4.4.0]dec-5-ene (TBD).
66. The method according to any one of Embodiments 62 to 65, wherein the hydrazine source is hydrazine hydrate and the additive is 1,5,7-triazabicyclo[4.4.0]dec-5-ene (TBD).
67. The method according to Embodiment 60, wherein the compound of Formula (13):

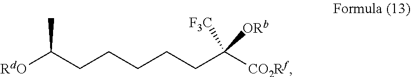

or a stereoisomer thereof, or a deuterated derivative of the compound of Formula (13) or its stereoisomer, or a salt of any of the foregoing, is prepared by converting a compound of Formula (14):

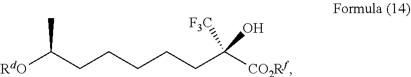

or a stereoisomer thereof, or a deuterated derivative of the compound of Formula (14) or its stereoisomer, or a salt of any of the foregoing, into the compound of Formula (13), or a stereoisomer thereof, or a deuterated derivative of the compound of Formula (13) or its stereoisomer, or a salt of any of the foregoing,
wherein:
   R^{b} is selected from benzyl (Bn), naphthylmethyl (Nap), biphenylmethyl, Ac, TFA, and Bz;
   R^{d} is selected from H, Bn, TBS, and TBDPS; and
   R^{f} is selected from Me, Et, and Bn.
68. The method according to Embodiment 67, wherein R^{b} is Bn, R^{d} is TBDPS, and R^{f} is Et.
69. The method according to Embodiment 67 or 68, wherein converting the compound of Formula (14), or a stereoisomer thereof, or a deuterated derivative of the compound of Formula (14) or its stereoisomer, or a salt of any of the foregoing, into the compound of Formula (13), or a stereoisomer thereof, or a deuterated derivative of the compound of Formula (13) or its stereoisomer, or a salt of any of the foregoing, is performed in the presence of an alkyl halide, an alkyl triflate, or an alkyl tosylate; a base; and a phase transfer catalyst.
70. The method according to Embodiment 69, wherein the alkyl halide is a benzyl halide.
71. The method according to Embodiment 70, wherein the benzyl halide is selected from benzyl chloride (BnCl), benzyl bromide (BnBr), and benzyl iodide (BnI).
72. The method according to any one of Embodiments 69 to 71, wherein the base is selected from lithium carbonate (Li₂CO₃), sodium carbonate (Na₂CO₃), potassium carbonate (K₂CO₃), cesium carbonate (Cs₂CO₃), sodium tert-butoxide (NaOt-Bu), and potassium tert-butoxide (KOt-Bu).
73. The method according to any one of Embodiments 69 to 72, wherein the phase transfer catalyst is selected from tetrabutylammonium chloride (TBAC), tetrabutylammonium bromide (TBAB), and tetrabutylammonium iodide (TBAI).
74. The method according to any one of Embodiments 69 to 73, wherein the alkyl halide is benzyl bromide (BnBr), the base is cesium carbonate (Cs₂CO₃), and the phase transfer catalyst is tetrabutylammonium iodide (TBAI).
75. The method according to Embodiment 67, wherein the compound of Formula (14):

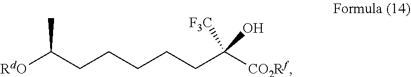

or a stereoisomer thereof, or a deuterated derivative of the compound of Formula (14) or its stereoisomer, or a salt of any of the foregoing, is prepared by converting a compound of Formula (15):

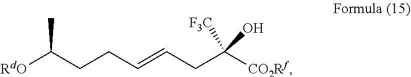

or a stereoisomer thereof, or a deuterated derivative of the compound of Formula (15) or its stereoisomer, or a salt of any of the foregoing, into the compound of Formula (14), or a stereoisomer thereof, or a deuterated derivative of the compound of Formula (14) or its stereoisomer, or a salt of any of the foregoing,
wherein:
   R^{d} is selected from H, Bn, TBS, and TBDPS; and
   R^{f} is selected from Me, Et, and Bn.
76. The method according to Embodiment 75, wherein R^{d} is TBDPS and R^{f} is Et.
77. The method according to Embodiment 75 or 76, wherein converting the compound of Formula (15), or a stereoisomer thereof, or a deuterated derivative of the compound of Formula (15) or its stereoisomer, or a salt of any of the foregoing, into the compound of Formula (14), or a stereoisomer thereof, or a deuterated derivative of the compound of Formula (15) or its stereoisomer, or a salt of any of the foregoing, is performed in the presence of hydrogen gas (H₂) and a palladium catalyst.
78. The method according to Embodiment 77, wherein the palladium catalyst is selected from palladium on carbon (Pd/C) and palladium on alumina (Pd/Al).
79. The method according to Embodiment 77 or 78, wherein the palladium catalyst is palladium on carbon (Pd/C).
80. The method according to Embodiment 75, wherein the compound of Formula (15):

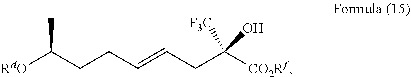

or a stereoisomer thereof, or a deuterated derivative of the compound of Formula (15) or its stereoisomer, or a salt of any of the foregoing, is prepared by combining a compound of Formula (16):

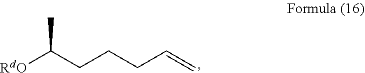

or a deuterated derivative or salt thereof, with a compound of Formula (17):

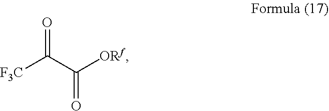

or a deuterated derivative or salt thereof, to produce the compound of Formula (15), or a stereoisomer thereof, or a deuterated derivative of the compound of Formula (15) or its stereoisomer, or a salt of any of the foregoing,
wherein:
   R^{d} is selected from H, Bn, TBS, and TBDPS; and
   R^{f} is selected from Me, Et, and Bn.
81. The method according to Embodiment 80, wherein R^{d} is TBDPS and R^{f} is Et.
82. The method according to Embodiment 80 or 81, wherein combining the compound of Formula (16), or a deuterated derivative or salt thereof, with the compound of Formula (17), or a deuterated derivative or salt thereof, is performed in the presence of a palladium salt, a phosphine ligand, and a silver salt.
83. The method according to Embodiment 82, wherein the palladium salt is selected from palladium(II) chloride (PdCl₂), palladium(II) acetate (Pd(OAc)₂), and bis(acetonitrile)dichloropalladium(II) (PdCl₂(MeCN)₂).
84. The method according to Embodiment 82 or 83, wherein the phosphine ligand is selected from (S)-(−)-(1,1′-binaphthalene-2,2′-diyl)bis(diphenylphosphine) ((S)-BINAP), (S)-(−)-2,2′-bis(di-p-tolylphosphino)-1,1′-binaphthyl ((S)-Tol-BINAP), and (S)-(−)-2,2′-bis[di(3,5-xylyl)phosphino]-1,1′-binaphthyl ((S)-Xyl-BINAP).
85. The method according to any one of Embodiments 82 to 84, wherein the silver salt is selected from silver hexafluoroantimonate (AgSbF₆) and silver tetrafluoroborate (AgBF₄).
86. The according to any one of Embodiments 82 to 85, wherein the palladium salt is palladium(II) chloride (PdCl₂), the phosphine ligand is (S)-(−)-(1,1′-binaphthalene-2,2′-diyl)bis(diphenylphosphine) ((S)-BINAP), and the silver salt is silver tetrafluoroborate (AgBF₄).
87. A method of preparing Compound I:

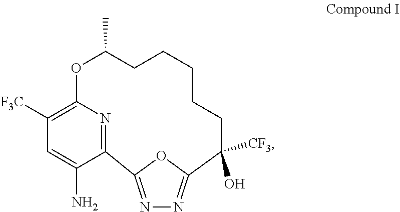 [SMILES: C[C@@H]1CCCCC[C@](O)(C(F)(F)F)C2=NN=C(O2)c2nc(c(C(F)(F)F)cc2N)O1]

or a stereoisomer thereof, or a deuterated derivative of the compound of Formula (1) or its stereoisomer, or a salt of any of the foregoing, comprising converting a compound of Formula (18):

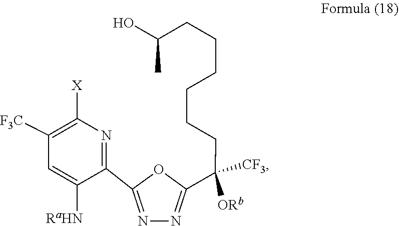

or a stereoisomer thereof, or a deuterated derivative of the compound of Formula (18) or its stereoisomer, or a salt of any of the foregoing, into Compound I, or a stereoisomer thereof, or a deuterated derivative of the compound of Formula (1) or its stereoisomer, or a salt of any of the foregoing,
wherein:
   X is selected from Cl, Br, I, —OSO₂R, and —SR,
   R is selected from Me, —CF₃, Ph, and 4-MePh;
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz;
88. The method according to Embodiment 87, wherein R^{a} is Boc and R^{b} is Bn.
89. The method according to Embodiment 87, wherein the method comprises converting a compound of Formula (2a):

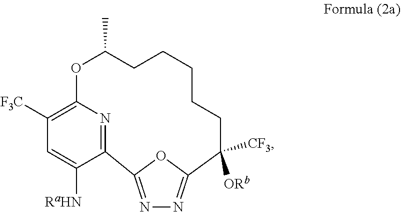

or a stereoisomer thereof, or a deuterated derivative of the compound of Formula (2a) or its stereoisomer, or a salt of any of the foregoing, into Compound I, or a stereoisomer thereof, or a deuterated derivative of the compound of Formula (1) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz;
90. The method according to Embodiment 89, wherein R^{a} is Boc and R^{b} is Bn.
91. The method according to Embodiment 87 or 89, wherein the method comprises converting a compound of Formula (3a):

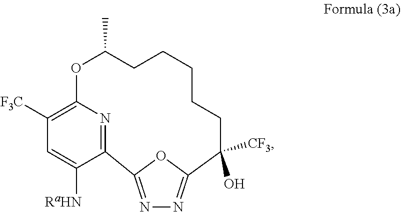

or a stereoisomer thereof, or a deuterated derivative of the compound of Formula (3a) or its stereoisomer, or a salt of any of the foregoing, into Compound I, or a stereoisomer thereof, or a deuterated derivative of the compound of Formula (1) or its stereoisomer, or a salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).
92. The method according to Embodiment 91, wherein R^{a} is Boc.
93. The method according to Embodiment 91 or 92, wherein converting the compound of Formula (3a), or a stereoisomer thereof, or a deuterated derivative of the compound of Formula (3a) or its stereoisomer, or a salt of any of the foregoing, into Compound I, or a stereoisomer thereof, or a deuterated derivative of the compound of Formula (1) or its stereoisomer, or a salt of any of the foregoing, is performed in the presence of an acid.
94. The method according to Embodiment 93, wherein the acid is selected from trifluoroacetic acid (TFA), hydrochloric acid (HCl), methanesulfonic acid (MsOH), phosphoric acid (H₃PO₄), and sulfuric acid (H₂SO₄).
95. The method according to Embodiment 93 or 94, wherein the acid is trifluoroacetic acid (TFA).
96. The method according to Embodiment 91, wherein the compound of Formula (3a):

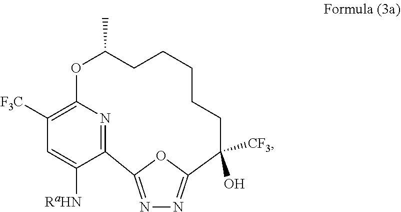

or a stereoisomer thereof, or a deuterated derivative of the compound of Formula (3a) or its stereoisomer, or a salt of any of the foregoing, is prepared by converting a compound of Formula (2a):

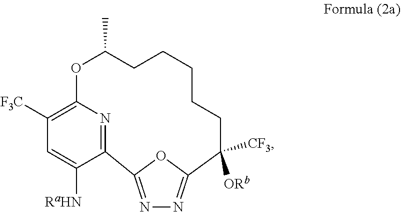

or a stereoisomer thereof, or a deuterated derivative of the compound of Formula (2a) or its stereoisomer, or a salt of any of the foregoing, into the compound of Formula (3a), or a stereoisomer thereof, or a deuterated derivative of the compound of Formula (3a) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz;
97. The method according to Embodiment 96, wherein R^{a} is Boc and R^{b} is Bn.
98. The method according to Embodiment 97, wherein converting the compound of Formula (2a), or a stereoisomer thereof, or a deuterated derivative of the compound of Formula (2a) or its stereoisomer, or a salt of any of the foregoing, into the compound of Formula (3a), or a stereoisomer thereof, or a deuterated derivative of the compound of Formula (3a) or its stereoisomer, or a salt of any of the foregoing is performed in the presence of hydrogen gas (H₂) and a palladium catalyst.
99. The method according to Embodiment 98, wherein the palladium catalyst is selected from palladium on carbon (Pd/C) and palladium on alumina (Pd/Al).
100. The method according to Embodiment 98 or 99, wherein the palladium catalyst is palladium on carbon (Pd/C).
101. The method according to Embodiment 89 or 96, wherein the compound of Formula (2a):

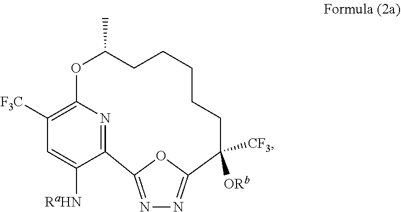

or a stereoisomer thereof, or a deuterated derivative of the compound of Formula (2a) or its stereoisomer, or a salt of any of the foregoing is prepared by converting a compound of Formula (18):

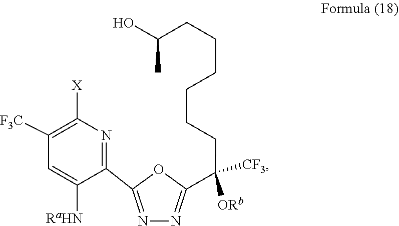

or a stereoisomer thereof, or a deuterated derivative of the compound of Formula (18) or its stereoisomer, or a salt of any of the foregoing, into the compound of Formula (2a), or a stereoisomer thereof, or a deuterated derivative of the compound of Formula (2a) or its stereoisomer, or a salt of any of the foregoing,
wherein:
   X is selected from Cl, Br, I, —OSO₂R, and —SO₂R,
   R is selected from Me, —CF₃, Ph, and 4-MePh;
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
102. The method according to Embodiment 87 or 101, wherein X is Cl, Br, or I; R^{a} is Boc; and R^{b} is Bn.
103. The method according to any one of Embodiments 87, 101, or 102, wherein X is Br, R^{a} is Boc, and R^{b} is Bn.
104. The method according to any one of Embodiments 87 and 101 to 103, wherein converting the compound of Formula (18), or a stereoisomer thereof, or a deuterated derivative of the compound of Formula (18) or its stereoisomer, or a salt of any of the foregoing, into the compound of Formula (2a), or a stereoisomer thereof, or a deuterated derivative of the compound of Formula (2a) or its stereoisomer, or a salt of any of the foregoing, is performed in the presence of a palladium catalyst and a base.
105. The method according to Embodiment 104, wherein the palladium catalyst is selected from methanesulfonato[9,9-dimethyl-4,5-bis(diphenylphosphino)xanthene](2′-methylamino-1,1′-biphenyl-2-yl)palladium(II), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II), methanesulfonato(2-(di-t-butylphosphino)-3-methoxy-6-methyl-2′,4′,6′-tri-i-propyl-1,1′-biphenyl)(2′-amino-1,1′-biphenyl-2-yl)palladium(II), methanesulfonato{(R)-(−)-1-[(S)-2-(dicyclohexylphosphino)ferrocenyl]ethyldi-t-butylphosphine}(2′-amino-1,1′-biphenyl-2-yl)palladium(II), methanesulfonato{N-[2-(di-1-adamantylphosphino)phenyl]morpholine}(2′-methylamino-1,1′-biphenyl-2-yl)palladium(II), cyclopenta-2,4-dien-1-yl(diphenyl)phosphane; iron; (N-methyl-2-phenyl-anilino)-methylsulfonyloxy-palladium(II), and methanesulfonato(2-dicyclohexylphosphino-2′,6′-bis(dimethylamino)-1,1′-biphenyl)(2′-amino-1,1′-biphenyl-2-yl)palladium(II).
106. The method according to Embodiment 104 or 105, wherein the base is selected from potassium phosphate tribasic (K₃PO₄), potassium carbonate (K₂CO₃), cesium carbonate (Cs₂CO₃), sodium tert-butoxide (KOt-Bu), and potassium tert-butoxide (KOt-Bu).
107. The method according to Embodiment 87 or 101, wherein X is —SO₂R, R is Me, R^{a} is Boc, and R^{b} is Bn.
108. The method according to Embodiment 107, wherein converting the compound of Formula (18), or a stereoisomer thereof, or a deuterated derivative of the compound of Formula (18) or its stereoisomer, or a salt of any of the foregoing, into the compound of Formula (2a), or a stereoisomer thereof, or a deuterated derivative of the compound of Formula (2a) or its stereoisomer, or a salt of any of the foregoing, is performed in the presence of a base.
109. The method according to Embodiment 108, wherein the base is potassium phosphate tribasic (K₃PO₄).
110. The method according to Embodiment 101, wherein the compound of Formula (18):

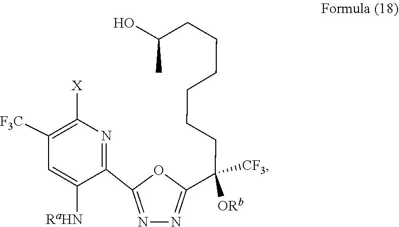

or a stereoisomer thereof, or a deuterated derivative of the compound of Formula (18) or its stereoisomer, or a salt of any of the foregoing, is prepared by converting a compound of Formula (19):

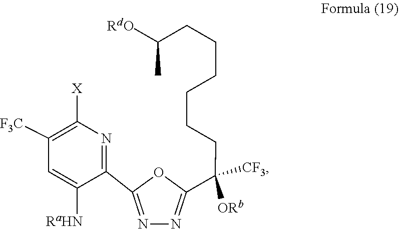

or a stereoisomer thereof, or a deuterated derivative of the compound of Formula (19) or its stereoisomer, or a salt of any of the foregoing, into the compound of Formula (18), or a stereoisomer thereof, or a deuterated derivative of the compound of Formula (18) or its stereoisomer, or a salt of any of the foregoing,
wherein:
   X is selected from Cl, Br, I, —OSO₂R, and —SO₂R,
   R is selected from Me, —CF₃, Ph, and 4-MePh;
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{b} is selected from benzyl (Bn), naphthylmethyl (Nap), biphenylmethyl, Ac, TFA, and Bz; and
   R^{d} is selected from H, Bn, TBS, and TBDPS.
111. The method according to Embodiment 110, wherein X is selected from Br and —SO₂R, R is Me, R^{a} is Boc, R^{b} is Bn, and R^{d} is TBDPS.
112. The method according to Embodiment 111, wherein converting the compound of Formula (19), or a stereoisomer thereof, or a deuterated derivative of the compound of Formula (19) or its stereoisomer, or a salt of any of the foregoing, into the compound of Formula (18), or a stereoisomer thereof, or a deuterated derivative of the compound of Formula (18) or its stereoisomer, or a salt of any of the foregoing, is performed in the presence of a quaternary ammonium fluoride or an inorganic fluoride salt.
113. The method according to Embodiment 111 or 112, wherein the quaternary ammonium fluoride is selected from tetramethylammonium fluoride (TMAF), tetraethylammonium fluoride (TEAF), and tetrabutylammonium fluoride (TBAF).
114. The method according to Embodiment 111 or 112, wherein the inorganic fluoride salt is selected from sodium fluoride (NaF), potassium fluoride (KF), and cesium fluoride (CF).
115. The method according to any one of Embodiments 111 to 113, wherein the quaternary ammonium fluoride is tetrabutylammonium fluoride (TBAF).
116. The method according to Embodiment 110, wherein the compound of Formula (19),

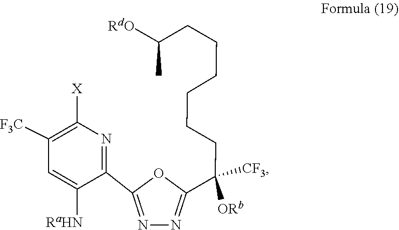

or a stereoisomer thereof, or a deuterated derivative of the compound of Formula (19) or its stereoisomer, or a salt of any of the foregoing, is prepared by converting the compound of Formula (20),

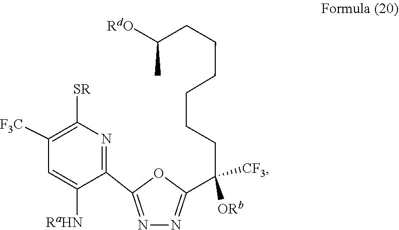

or a stereoisomer thereof, or a deuterated derivative of the compound of Formula (20) or its stereoisomer, or a salt of any of the foregoing, into the compound of Formula (19), or a stereoisomer thereof, or a deuterated derivative of the compound of Formula (19) or its stereoisomer, or a salt of any of the foregoing,
wherein:
   X is —SO₂R,
   R is selected from Me, —CF₃, Ph, and 4-MePh;
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{b} is selected from benzyl (Bn), naphthylmethyl (Nap), biphenylmethyl, Ac, TFA, and Bz; and
   R^{d} is selected from H, Bn, TBS, and TBDPS.
117. The method according to Embodiment 116, wherein X is —SO₂Me, R is Me, R^{a} is Boc, R^{b} is Bn, and R^{d} is TBDPS.
118. The method according to Embodiment 116 or 117, wherein converting the compound of Formula (20), or a deuterated derivative thereof, or a salt of any of the foregoing, into the compound of Formula (19), or a deuterated derivative thereof, or a salt of any of the foregoing, is performed in the presence of ruthenium(III) chloride (RuCl₃), sodium periodate (NaIO₄), and water (H₂O).
119. The method according to Embodiment 110, wherein the compound of Formula (19):

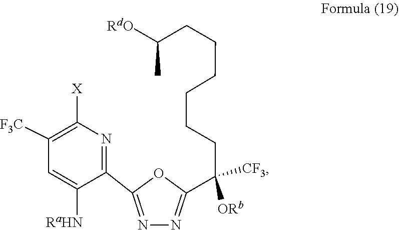

or a stereoisomer thereof, or a deuterated derivative of the compound of Formula (19) or its stereoisomer, or a salt of any of the foregoing, is prepared by converting a compound of Formula (21):

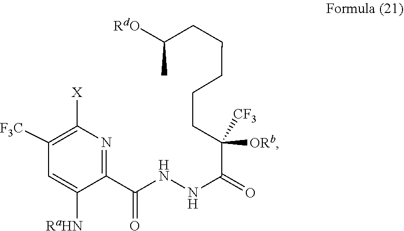

or a stereoisomer thereof, or a deuterated derivative of the compound of Formula (21) or its stereoisomer, or a salt of any of the foregoing, into the compound of Formula (19), or a stereoisomer thereof, or a deuterated derivative of the compound of Formula (19) or its stereoisomer, or a salt of any of the foregoing,
wherein:
   X is selected from Cl, Br, I, —OSO₂R, and —SR,
   R is selected from Me, —CF₃, Ph, and 4-MePh;
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{b} is selected from benzyl (Bn), naphthylmethyl (Nap), biphenylmethyl, Ac, TFA, and Bz; and
   R^{d} is selected from H, Bn, TBS, and TBDPS.
120. The method according to Embodiment 119, wherein X is selected from Br and —SR, R is Me, R^{a} is Boc, R^{b} is Bn, and R^{d} is TBDPS.
121. The method according to Embodiment 119 or 120, wherein converting the compound of Formula (21), or a stereoisomer thereof, or a deuterated derivative of the compound of Formula (21) or its stereoisomer, or a salt of any of the foregoing, into the compound of Formula (19), or a stereoisomer thereof, or a deuterated derivative of the compound of Formula (19) or its stereoisomer, or a salt of any of the foregoing, is performed in the presence of a sulfonyl chloride and amine base, and optionally in the presence of an additive.
122. The method according to Embodiment 121, wherein the sulfonyl chloride is p-toluenesulfonyl chloride (TsCl).
123. The method according to Embodiment 121 or 122, wherein the amine base is N,N-diisopropylethylamine (DIPEA).
124. The method according to any one of 121 to 123, wherein the additive is 1,4-diazabicyclo[2.2.2]octane (DABCO).
125. The method according to any one of Embodiments 121 to 124, wherein the sulfonyl chloride is p-toluenesulfonyl chloride (TsCl), the amine base is N,N-diisopropylethylamine (DIPEA), and the additive is 1,4-diazabicyclo[2.2.2]octane (DABCO).
126. The method according to Embodiment 119, wherein the compound of Formula (21):

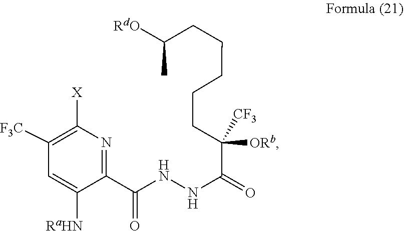

or a stereoisomer thereof, or a deuterated derivative of the compound of Formula (21) or its stereoisomer, or a salt of any of the foregoing, is prepared by combining a compound of Formula (22):

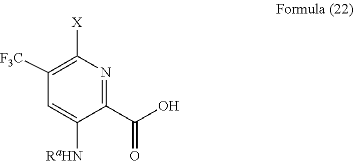

or a deuterated derivative or salt thereof, with a compound of Formula (23):

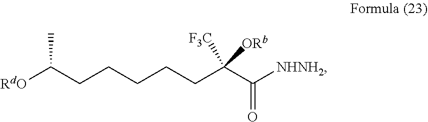

or a stereoisomer thereof, or a deuterated derivative of the compound of Formula (23) or its stereoisomer, or a salt of any of the foregoing, to produce the compound of Formula (21), or a stereoisomer thereof, or a deuterated derivative of the compound of Formula (21) or its stereoisomer, or a salt of any of the foregoing,
wherein:
   X is selected from Cl, Br, I, —OSO₂R, and —SR,
   R is selected from Me, —CF₃, Ph, and 4-MePh;
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns);
   R^{b} is selected from benzyl (Bn), naphthylmethyl (Nap), biphenylmethyl, Ac, TFA, and Bz; and
   R^{d} is selected from H, Bn, TBS, and TBDPS.
127. The method according to Embodiment 126, wherein X is selected from Br and —SR, R is Me, R^{a} is Boc, R^{b} is Bn, and R^{d} is TBDPS.
128. The method according to Embodiment 126 or 127, wherein combining the compound of Formula (22), or a deuterated derivative or salt thereof, with the compound of Formula (23), or a stereoisomer thereof, or a deuterated derivative of the compound of Formula (23) or its stereoisomer, or a salt of any of the foregoing, is performed in the presence of a peptide coupling agent and an amine base.
129. The method according to Embodiment 128, wherein the peptide coupling agent is propylphosphonic anhydride (T3P).
130. The method according to Embodiment 128 or 129, wherein the amine base is N-methylmorpholine (NMM).
131. The method according to any one of Embodiments 128 to 130, wherein the peptide coupling agent is propylphosphonic anhydride (T3P) and the amine base is N-methylmorpholine (NMM).
132. The method according to Embodiment 126, wherein the compound of Formula (23):

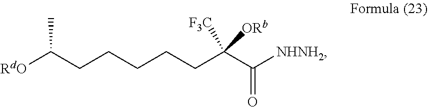

or a stereoisomer thereof, or a deuterated derivative of the compound of Formula (23) or its stereoisomer, or a salt of any of the foregoing, is prepared by converting a compound of Formula (24):

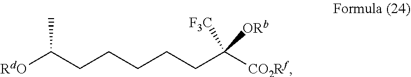

or a stereoisomer thereof, or a deuterated derivative of the compound of Formula (24) or its stereoisomer, or a salt of any of the foregoing, into the compound of Formula (23), or a salt thereof,
wherein:
   R^{b} is selected from benzyl (Bn), naphthylmethyl (Nap), biphenylmethyl, Ac, TFA, and Bz;
   R^{d} is selected from H, Bn, TBS, and TBDPS; and
   R^{f} is selected from Me, Et, and Bn.
133. The method according to Embodiment 132, wherein converting the compound of Formula (24), or a stereoisomer thereof, or a deuterated derivative of the compound of Formula (24) or its stereoisomer, or a salt of any of the foregoing, into the compound of Formula (23), or a stereoisomer thereof, or a deuterated derivative of the compound of Formula (23) or its stereoisomer, or a salt of any of the foregoing, is performed in the presence of a hydrazine source and optionally an additive.
134. The method according to Embodiment 133, wherein the hydrazine source is selected from hydrazine hydrate, hydrazine monohydrochloride, hydrazine dihydrochloride, and hydrazine sulfate salt.
135. The method according to Embodiment 133 or 134, wherein the additive is selected from guanidine bases.
136. The method according to any one of Embodiments 133 to 135, wherein the additive is 1,5,7-triazabicyclo[4.4.0]dec-5-ene (TBD).
137. The method according to any one of Embodiments 133 to 136, wherein the hydrazine source is hydrazine hydrate and the additive is 1,5,7-triazabicyclo[4.4.0]dec-5-ene (TBD).
138. The method according to Embodiment 132, wherein the compound of Formula (24):

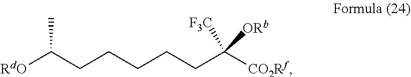

or a stereoisomer thereof, or a deuterated derivative of the compound of Formula (24) or its stereoisomer, or a salt of any of the foregoing, is prepared by converting a compound of Formula (25):

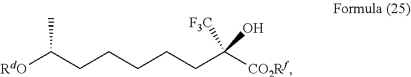

or a stereoisomer thereof, or a deuterated derivative of the compound of Formula (25) or its stereoisomer, or a salt of any of the foregoing, or a salt of any of the foregoing, into the compound of Formula (24), or a stereoisomer thereof, or a deuterated derivative of the compound of Formula (24) or its stereoisomer, or a salt of any of the foregoing,
wherein:
   R^{b} is selected from benzyl (Bn), naphthylmethyl (Nap), biphenylmethyl, Ac, TFA, and Bz;
   R^{d} is selected from H, Bn, TBS, and TBDPS; and
   R^{f} is selected from Me, Et, and Bn.
139. The method according to Embodiment 138, wherein R^{b} is Bn, R^{d} is TBDPS, and R^{f} is Et.
140. The method according to Embodiment 138 or 139, wherein converting the compound of Formula (25), or a stereoisomer thereof, or a deuterated derivative of the compound of Formula (25) or its stereoisomer, or a salt of any of the foregoing, into the compound of Formula (24), or a stereoisomer thereof, or a deuterated derivative of the compound of Formula (24) or its stereoisomer, or a salt of any of the foregoing, is performed in the presence of an alkyl halide, an alkyl triflate, or an alkyl tosylate; a base, and a phase transfer catalyst.
141. The method according to Embodiment 140, wherein the alkyl halide is a benzyl halide.
142. The method according to Embodiment 141, wherein the benzyl halide is selected from benzyl chloride (BnCl), benzyl bromide (BnBr), and benzyl iodide (BnI).
143. The method according to any one of Embodiments 140 to 142, wherein the base is selected from cesium carbonate (Cs₂CO₃), potassium carbonate (K₂CO₃), sodium tert-butoxide (NaOt-Bu).
144. The method according to any one of Embodiments 140 to 143, wherein the phase transfer catalyst is selected from tetrabutylammonium chloride (TBAC), tetrabutylammonium bromide (TBAB), and tetrabutylammonium iodide (TBAI).
145. The method according to any one of Embodiments 140 to 144, wherein the alkyl halide is benzyl bromide (BnBr), wherein the base is cesium carbonate (Cs₂CO₃), and the phase transfer catalyst is tetrabutylammonium iodide (TBAI).
146. The method according to Embodiment 138, wherein the compound of Formula (25):

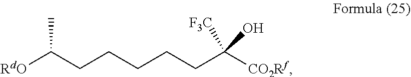

or a stereoisomer thereof, or a deuterated derivative of the compound of Formula (25) or its stereoisomer, or a salt of any of the foregoing, is prepared by converting a compound of Formula (26):

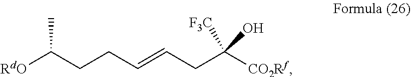

or a stereoisomer thereof, or a deuterated derivative of the compound of Formula (26) or its stereoisomer, or a salt of any of the foregoing, into the compound of Formula (25), or a stereoisomer thereof, or a deuterated derivative of the compound of Formula (25) or its stereoisomer, or a salt of any of the foregoing,
wherein:
   R^{d} is selected from H, Bn, TBS, and TBDPS; and
   R^{f} is selected from Me, Et, and Bn.
147. The method according to Embodiment 146, wherein R^{d} is TBDPS and R^{f} is Et.
148. The method according to Embodiment 146 or 147, wherein converting the compound of Formula (26), or a deuterated derivative thereof, or a salt of any of the foregoing, into the compound of Formula (25), or a deuterated derivative thereof, or a salt of any of the foregoing, is performed in the presence of hydrogen gas (H₂) and a palladium catalyst.
149. The method according to Embodiment 148, wherein the palladium catalyst is selected from palladium on carbon (Pd/C) and palladium on alumina (Pd/Al).
150. The method according to Embodiment 148 or 149, wherein the palladium catalyst is palladium on carbon (Pd/C).
151. The method according to Embodiment 146, wherein the compound of Formula (26):

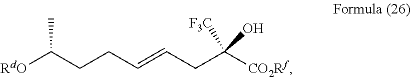

or a stereoisomer thereof, or a deuterated derivative of the compound of Formula (26) or its stereoisomer, or a salt of any of the foregoing, is prepared by combining a compound of Formula (27):

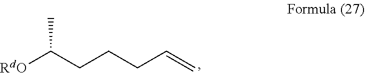

or a stereoisomer thereof, or a deuterated derivative of the compound of Formula (27) or its stereoisomer, with a compound of Formula (17),

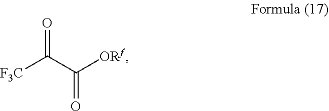

or a deuterated derivative thereof, to produce the compound of Formula (26), or a stereoisomer thereof, or a deuterated derivative of the compound of Formula (26) or its stereoisomer, or a salt of any of the foregoing,
wherein:
   R^{d} is selected from H, Bn, TBS, and TBDPS; and
   R^{f} is selected from Me, Et, and Bn.
152. The method according to Embodiment 151, wherein R^{d} is TBDPS and R^{f} is Et.
153. The method according to Embodiment 151, wherein combining the compound of Formula (27), or a stereoisomer thereof, or a deuterated derivative of the compound of Formula (27) or its stereoisomer, with the compound of Formula (17), or a deuterated derivative thereof, is performed in the presence of a palladium salt, a phosphine ligand, and a silver salt.
154. The method according to Embodiment 153, wherein the palladium salt is selected from palladium(II) chloride (PdCl₂), palladium(II) acetate (Pd(OAc)₂), and bis(acetonitrile)dichloropalladium(II) (PdCl₂(MeCN)₂).
155. The method according to Embodiment 153 or 154, wherein the phosphine ligand is selected from
   (S)-(−)-(1,1′-binaphthalene-2,2′-diyl)bis(diphenylphosphine) ((S)-BINAP), (S)-(−)-2,2′-bis(di-p-tolylphosphino)-1,1′-binaphthyl ((S)-Tol-BINAP), and (S)-(−)-2,2′-bis[di(3,5-xylyl)phosphino]-1,1′-binaphthyl ((S)-Xyl-BINAP).
156. The method according to any one of Embodiments 153 to 155, wherein the silver salt is selected from silver hexafluoroantimonate (AgSbF₆) and silver tetrafluoroborate (AgBF₄).
157. The according to any one of Embodiments 153 to 156, wherein the palladium salt is palladium(II) chloride (PdCl₂), the phosphine ligand is (S)-(−)-(1,1′-binaphthalene-2,2′-diyl)bis(diphenylphosphine) ((S)-BINAP), and the silver salt is silver tetrafluoroborate (AgBF₄).
158. The method according to Embodiment 126, wherein the compound of Formula (22):

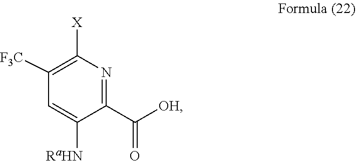

or a deuterated derivative or salt thereof, is prepared by converting a compound of Formula (28):

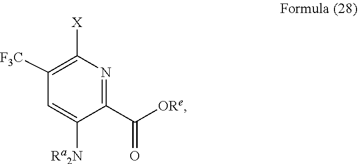

or a deuterated derivative or salt thereof, into the compound of Formula (22), or a deuterated derivative or salt thereof,
wherein:
   X is —SR,
   R is selected from Me, —CF₃, Ph, and 4-MePh;
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{e} is selected from Me, Et, n-Pr, i-Pr, and t-Bu.
159. The method according to Embodiment 158, wherein X is —SMe, R^{a} is Boc and R^{e} is Me.
160. The method according to Embodiment 158 or 159, wherein converting the compound of Formula (28), or a deuterated derivative or salt thereof, into the compound of Formula (22), or a deuterated derivative or salt thereof, is performed in the presence of aqueous hydroxide base.
161. The method according to Embodiment 160, wherein the aqueous hydroxide base is selected from aqueous lithium hydroxide (LiOH), aqueous sodium hydroxide (NaOH), and aqueous potassium hydroxide (KOH).
162. The method according to Embodiment 160 or 161, wherein the aqueous hydroxide base is aqueous lithium hydroxide (LiOH).
163. The method according to Embodiment 158, wherein the compound of Formula (28):

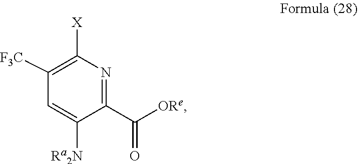

or a deuterated derivative or salt thereof, is prepared by converting a compound of Formula (29):

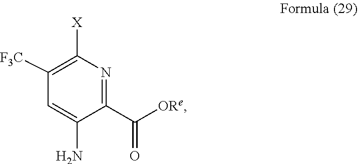

into the compound of Formula (29), or a deuterated derivative or salt thereof, wherein:
   X is —SR,
   R is selected from Me, —CF₃, Ph, and 4-MePh;
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{e} is selected from Me, Et, n-Pr, i-Pr, and t-Bu.
164. The method according to Embodiment 163, wherein X is —SMe, R^{a} is Boc, and R^{e} is Me.
165. The method according to Embodiment 163 or 164, wherein converting the compound of Formula (29), or a deuterated derivative or salt thereof, into the compound of Formula (28), or a deuterated derivative thereof, is performed in the presence of di-tert-butyl dicarbonate (Boc₂O) and 4-(dimethylamino)pyridine (DMAP).
166. The method according to Embodiment 163, wherein the compound of Formula (29):

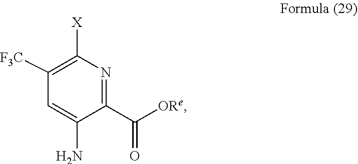

or a deuterated derivative or salt thereof, is prepared by converting a compound of Formula (30):

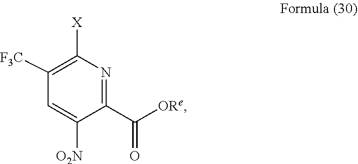

into the compound of Formula (29), or a deuterated derivative or salt thereof,
wherein:
   X is —SR,
   R is selected from Me, —CF₃, Ph, and 4-MePh; and
   R^{e} is selected from Me, Et, n-Pr, i-Pr, and t-Bu.
167. The method according to Embodiment 166, wherein X is —SMe and R^{e} is Me.
168. The method according to Embodiment 166 and 167, wherein converting the compound of Formula (30), or a deuterated derivative or salt thereof, into the compound of Formula (29), or a deuterated derivative or salt thereof, is performed in the presence of reducing conditions.
169. The method according to Embodiment 168, wherein the reducing conditions are iron (Fe) and aqueous ammonium chloride.
170. The method according to Embodiment 166, wherein the compound of Formula (30):

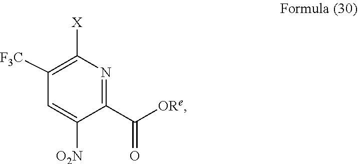

or a deuterated derivative or salt thereof, is prepared by converting a compound of Formula (31):

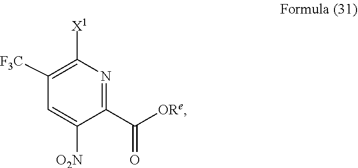

into the compound of Formula (30), or a deuterated derivative or salt thereof,
wherein:
   X is —SR
   R is selected from Me, —CF₃, Ph, and 4-MePh; and
   X¹ is Cl, Br, or I.
171. The method according to Embodiment 170, wherein X is —SMe, X¹ is Cl, and R^{e} is Me.
172. The method according to Embodiment 170 or 171, wherein converting the compound of Formula (31), or a deuterated derivative or salt thereof, into the compound of Formula (30), or a deuterated derivative or salt thereof, is performed in the presence of a thiolate salt.
173. The method according to Embodiment 172, wherein the thiolate salt is sodium methanethiolate (NaSMe).
174. A method of preparing Compound I:

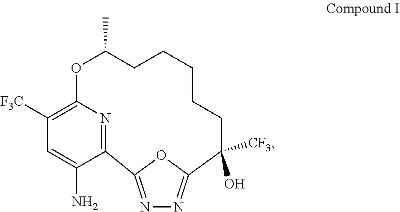 [SMILES: C[C@@H]1CCCCC[C@](O)(C(F)(F)F)C2=NN=C(O2)c2nc(c(C(F)(F)F)cc2N)O1]

or a stereoisomer thereof, or a deuterated derivative of Compound I or its stereoisomer, or a salt of any of the foregoing, comprising converting a compound of Formula (32):

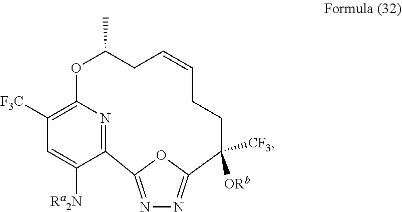

or a stereoisomer thereof, or a deuterated derivative of the compound of Formula (32) or its stereoisomer, or a salt of any of the foregoing, into Compound I, or a stereoisomer thereof, or a deuterated derivative of Compound I or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns) or N(R^{a})₂ is NO₂; and
   R^{b} is selected from benzyl (Bn), naphthylmethyl (Nap), biphenylmethyl, Ac, TFA, and Bz.
174a. A method of preparing Compound I:

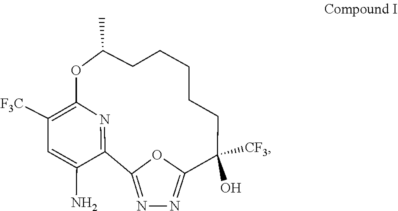 [SMILES: C[C@@H]1CCCCC[C@](O)(C(F)(F)F)C2=NN=C(O2)c2nc(c(C(F)(F)F)cc2N)O1]

or a stereoisomer thereof, or a deuterated derivative of Compound I or its stereoisomer, or a salt of any of the foregoing, comprising converting a compound of Formula (32a):

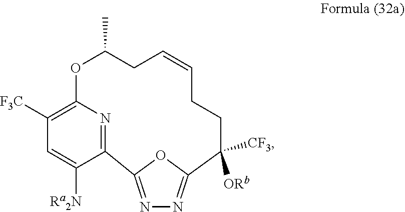

or a stereoisomer thereof, or a deuterated derivative of the compound of Formula (32a) or its stereoisomer, or a salt of any of the foregoing, into Compound I, or a stereoisomer thereof, or a deuterated derivative of Compound I or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
175. The method according to Embodiment 174, wherein N(R^{a})₂ is NO₂, and R^{b} is Bn.
175a. The method according to any one of Embodiments 174 to 175, wherein each R^{a} is independently H or Boc and R^{b} is Bn.
176. The method according to Embodiment 174a, wherein the method comprises converting a compound of Formula (33):

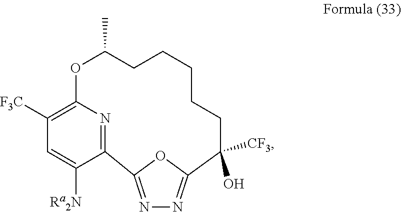

or a stereoisomer thereof, or a deuterated derivative of the compound of Formula (33) or its stereoisomer, or a salt of any of the foregoing, into Compound I, or a stereoisomer thereof, or a deuterated derivative of Compound I or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
177. The method according to Embodiment 176, wherein each R^{a} is independently H or Boc.
178. The method according to Embodiment 177, wherein converting the compound of Formula (33), or a stereoisomer thereof, or a deuterated derivative of the compound of Formula (33) or its stereoisomer, or a salt of any of the foregoing, into Compound I, or a stereoisomer thereof, or a deuterated derivative of Compound I or its stereoisomer, or a salt of any of the foregoing, is performed in the presence of an acid.
179. The method according to Embodiment 177 or 178, wherein the acid is selected from trifluoroacetic acid (TFA), hydrochloric acid (HCl), methanesulfonic acid (MsOH), phosphoric acid (H₃PO₄), and sulfuric acid (H₂SO₄).
180. The method according to any one of Embodiments 177 to 179, wherein the acid is trifluoroacetic acid (TFA).
181. The method according to Embodiment 176, wherein the compound of Formula (33):

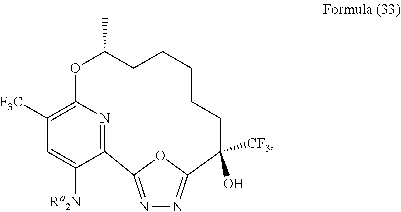

or a stereoisomer thereof, or a deuterated derivative of the compound of Formula (33) or its stereoisomer, or a salt of any of the foregoing, is prepared by converting a compound of Formula (32):

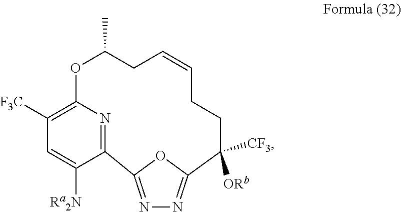

or a stereoisomer thereof, or a deuterated derivative of the compound of Formula (32) or its stereoisomer, or a salt of any of the foregoing, into the compound of Formula (33), or a stereoisomer thereof, or a deuterated derivative of the compound of Formula (33) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
   R^{b} is selected from benzyl (Bn), naphthylmethyl (Nap), biphenylmethyl, Ac, TFA, and Bz.
181a. The method according to Embodiment 176, wherein the compound of Formula (33):

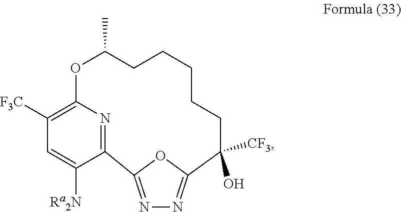

or a stereoisomer thereof, or a deuterated derivative of the compound of Formula (33) or its stereoisomer, or a salt of any of the foregoing, is prepared by converting a compound of Formula (32):

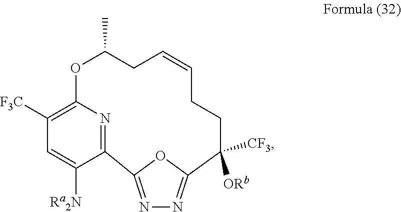

or a stereoisomer thereof, or a deuterated derivative of the compound of Formula (32) or its stereoisomer, or a salt of any of the foregoing, into the compound of Formula (33), or a stereoisomer thereof, or a deuterated derivative of the compound of Formula (33) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
182. The method according to Embodiment 181 or 181a, wherein each R^{a} is independently H or Boc and R^{b} is Bn.
183. The method according to Embodiment 182, wherein converting the compound of Formula (32), or a stereoisomer thereof, or a deuterated derivative of the compound of Formula (32) or its stereoisomer, or a salt of any of the foregoing, into the compound of Formula (33), or a stereoisomer thereof, or a deuterated derivative of the compound of Formula (33) or its stereoisomer, or a salt of any of the foregoing is performed in the presence of hydrogen gas (H₂) and a palladium catalyst.
184. The method according to Embodiment 183, wherein the palladium catalyst is selected from palladium on carbon (Pd/C) and palladium on alumina (Pd/Al).
185. The method according to Embodiment 183 or 184, wherein the palladium catalyst is palladium on carbon (Pd/C).
186. The method according to Embodiment 174, wherein the compound of Formula (32):

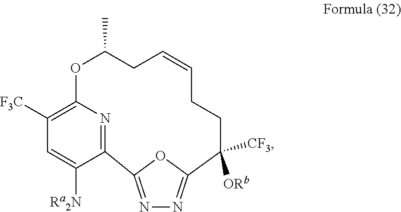

or a stereoisomer thereof, or a deuterated derivative of the compound of Formula (32) or its stereoisomer, or a salt of any of the foregoing, is prepared by converting a compound of Formula (34):

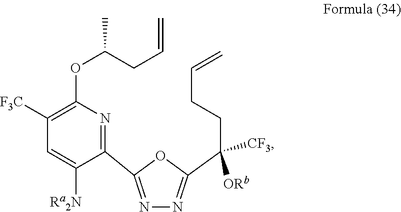

or a stereoisomer thereof, or a deuterated derivative of the compound of Formula (34) or its stereoisomer, into the compound of Formula (32), or a stereoisomer thereof, or a deuterated derivative of the compound of Formula (32) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
   R^{b} is selected from benzyl (Bn), naphthylmethyl (Nap), biphenylmethyl, Ac, TFA, and Bz.
186a. The method according to Embodiment 174, wherein the compound of Formula (32):

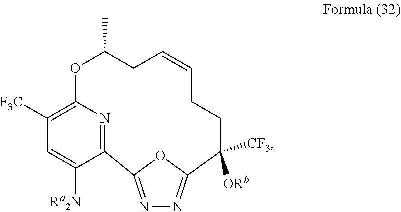

or a stereoisomer thereof, or a deuterated derivative of the compound of Formula (32) or its stereoisomer, or a salt of any of the foregoing, is prepared by converting a compound of Formula (36a):

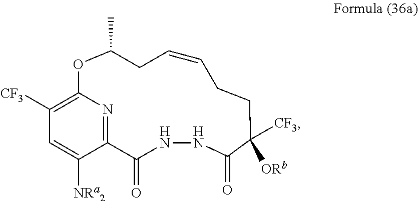

or a stereoisomer thereof, or a deuterated derivative of the compound of Formula (36a) or its stereoisomer, into the compound of Formula (32), or a stereoisomer thereof, or a deuterated derivative of the compound of Formula (32) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or NR^{a}₂ is NO₂; and
   R^{b} is selected from benzyl (Bn), naphthylmethyl (Nap), biphenylmethyl, Ac, TFA, and Bz.
186b. The method according to Embodiment 176 or 181, wherein the compound of Formula (32):

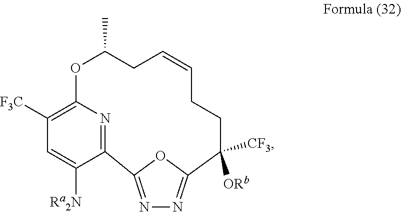

or a stereoisomer thereof, or a deuterated derivative of the compound of Formula (32) or its stereoisomer, or a salt of any of the foregoing, is prepared by converting a compound of Formula (34):

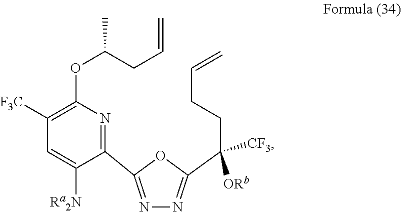

or a stereoisomer thereof, or a deuterated derivative of the compound of Formula (34) or its stereoisomer, into the compound of Formula (32), or a stereoisomer thereof, or a deuterated derivative of the compound of Formula (32) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
187. The method according to Embodiment 186 or 186a, wherein N(R^{a})₂ is NO₂ and R^{b} is Bn.
187a. The method according to Embodiment 186b, wherein each R^{a} is independently H or Boc and R^{b} is Bn.
188. The method according to any one of Embodiments 186 to 187a, wherein converting the compound of Formula (34), or a stereoisomer thereof, or a deuterated derivative of the compound of Formula (34) or its stereoisomer, into the compound of Formula (32), or a stereoisomer thereof, or a deuterated derivative of the compound of Formula (32) or its stereoisomer, or a salt of any of the foregoing, is performed in the presence of a ruthenium catalyst.
189. The method according to Embodiment 188, wherein the ruthenium catalyst is selected from dichloro(3-phenyl-1H-inden-1-ylidene)bis(tricyclohexylphosphine)ruthenium(II) (Umicore M101 Ru-catalyst), benzylidene-bis(tricyclohexylphosphine)dichlororuthenium, dichloro(2-isopropoxyphenylmethylene) (tricyclohexylphosphine)ruthenium(II), dichloro[1,3-bis(2,6-isopropylphenyl)-2-imidazolidinylidene](2-isopropoxyphenylmethylene)ruthenium(II), (1,3-bis-(2,4,6-trimethylphenyl)-2-imidazolidinylidene)dichloro(o-isopropoxyphenylmethylene) ruthenium, and dichloro[1,3-bis(2,4,6-trimethylphenyl)-2-imidazolidinylidene](3-phenyl-1H-inden-1-ylidene)(triphenylphosphine)ruthenium(II).
190. The method according to Embodiment 188 or 189, wherein the ruthenium catalyst is Umicore M101 Ru-catalyst.
190a. The method according to Embodiment 186a, wherein converting the compound of Formula (36a), or a stereoisomer thereof, or a deuterated derivative of the compound of Formula (36a) or its stereoisomer, into the compound of Formula (32), or a stereoisomer thereof, or a deuterated derivative of the compound of Formula (32) or its stereoisomer, or a salt of any of the foregoing, is performed in the presence of a sulfonyl chloride and an amine base, and optionally in the presence of an additive.
190b. The method according to Embodiment 190a, wherein the sulfonyl chloride is p-toluenesulfonyl chloride (TsCl).
190c. The method according to Embodiment 190a and 190b, wherein the amine base is N,N-diisopropylethylamine (DIPEA).
190d. The method according to any one of Embodiments 190a to 190c, wherein the additive is 1,4-diazabicyclo[2.2.2]octane (DABCO).
190f. The method according to any one of Embodiments 190a to 190d, wherein the compound of Formula (36a):

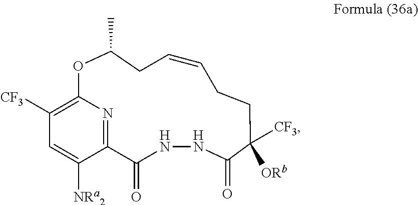

or a stereoisomer thereof, or a deuterated derivative of the compound of Formula (36a) or its stereoisomer, or a salt of any of the foregoing, is prepared by converting a compound of Formula (36b):

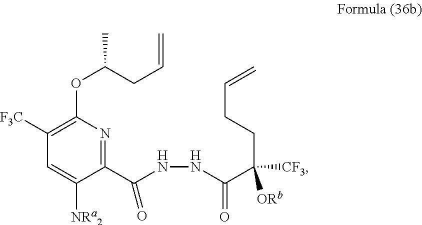

or a stereoisomer thereof, or a deuterated derivative of the compound of Formula (36b) or its stereoisomer, into the compound of Formula (36a), or a stereoisomer thereof, or a deuterated derivative of the compound of Formula (36a) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or NR^{a}₂ is NO₂; and
   R^{b} is selected from benzyl (Bn), naphthylmethyl (Nap), biphenylmethyl, Ac, TFA, and Bz.
190g. The method according to any one of Embodiments 190a to 190f, wherein N(R^{a})₂ is NO₂.
191. The method according to Embodiment 186, wherein the compound of Formula (34):

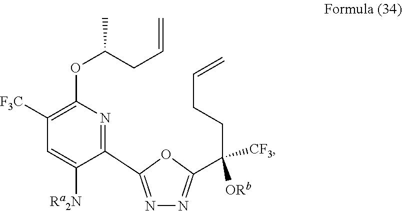

or a stereoisomer thereof, or a deuterated derivative of the compound of Formula (34) or its stereoisomer, is prepared by converting a compound of Formula (35):

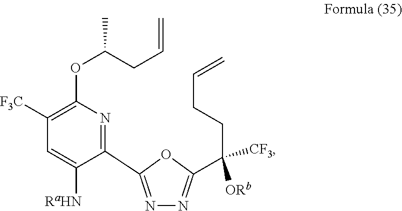

or a stereoisomer thereof, or a deuterated derivative of the compound of Formula (35) or its stereoisomer, or a salt of any of the foregoing, into the compound of Formula (34), or a stereoisomer thereof, or a deuterated derivative of the compound of Formula (34) or its stereoisomer,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
192. The method according to Embodiment 191, wherein R^{a} is Boc and R^{b} is Bn.
193. The method according to Embodiment 191 and 192, wherein converting the compound of Formula (35), or a stereoisomer thereof, or a deuterated derivative of the compound of Formula (35) or its stereoisomer, or a salt of any of the foregoing, into the compound of Formula (34), or a stereoisomer thereof, or a deuterated derivative of the compound of Formula (34) or its stereoisomer, is performed in the presence of di-tert-butyl dicarbonate (Boc₂O), N,N-diisopropylethylamine (DIPEA), and 4-(dimethylamino)pyridine (DMAP).
194. The method according to Embodiment 191, wherein the compound of Formula (35):

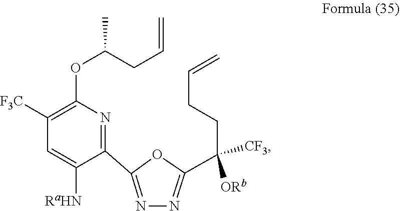

or a stereoisomer thereof, or a deuterated derivative of the compound of Formula (35) or its stereoisomer, or a salt of any of the foregoing, is prepared by converting a compound of Formula (36):

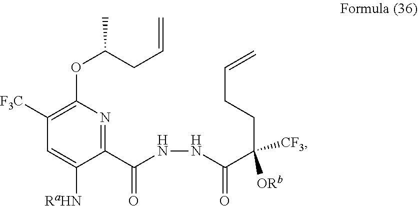

or a stereoisomer thereof, or a deuterated derivative of the compound of Formula (36) or its stereoisomer, or a salt of any of the foregoing, into the compound of Formula (35), or a stereoisomer thereof, or a deuterated derivative of the compound of Formula (35) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
195. The method according to Embodiment 194, wherein R^{a} is Boc and R^{b} is Bn.
196. The method according to Embodiment 194 and 195, wherein converting the compound of Formula (36), or a stereoisomer thereof, or a deuterated derivative of the compound of Formula (36) or its stereoisomer, or a salt of any of the foregoing, into the compound of Formula (35), or a stereoisomer thereof, or a deuterated derivative of the compound of Formula (35) or its stereoisomer, or a salt of any of the foregoing, is performed in the presence of a sulfonyl chloride and an amine base, and optionally in the presence of an additive.
197. The method according to Embodiment 196, wherein the sulfonyl chloride is p-toluenesulfonyl chloride (TsCl).
198. The method according to Embodiment 196 and 197, wherein the amine base is N,N-diisopropylethylamine (DIPEA).
199. The method according to any one of Embodiments 196 to 198, wherein the additive is 1,4-diazabicyclo[2.2.2]octane (DABCO).
200. The method according to any one of Embodiments 196 to 199, wherein the sulfonyl chloride is p-toluenesulfonyl chloride (TsCl), the amine base is N,N-diisopropylethylamine (DIPEA), and the additive is 1,4-diazabicyclo[2.2.2]octane (DABCO).
201. The method according to Embodiment 194, wherein the compound of Formula (36):

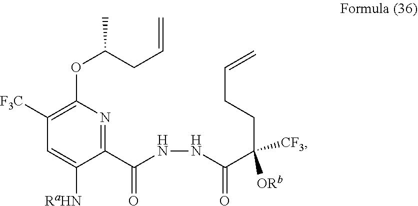

or a stereoisomer thereof, or a deuterated derivative of the compound of Formula (36) or its stereoisomer, or a salt of any of the foregoing, is prepared by combining a compound of Formula (37):

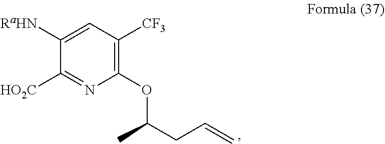

or a deuterated derivative or salt thereof, with a compound of Formula (38):

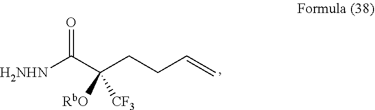

or a stereoisomer thereof, or a deuterated derivative of the compound of Formula (38) or its stereoisomer, or a salt of any of the foregoing, to produce the compound of Formula (36), or a stereoisomer thereof, or a deuterated derivative of the compound of Formula (36) or its stereoisomer, or a salt of any of the foregoing,
wherein:
   R^{a} is selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or NR^{a}₂ is NO₂; and
   R^{b} is selected from benzyl (Bn), naphthylmethyl (Nap), biphenylmethyl, Ac, TFA, and Bz.
201a. The method according to Embodiment 186 or 187, wherein the compound of Formula (34):

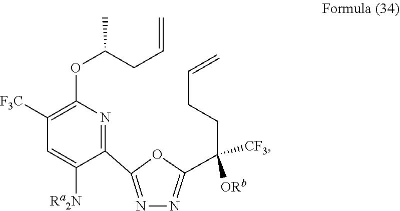

or a stereoisomer thereof, or a deuterated derivative of the compound of Formula (34) or its stereoisomer, or a salt of any of the foregoing, is prepared by combining a compound of Formula (37a):

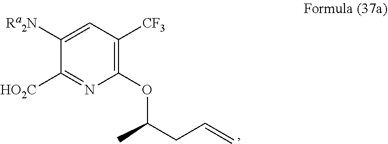

or a deuterated derivative or salt thereof, with a compound of Formula (38):

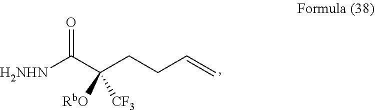

or a stereoisomer thereof, or a deuterated derivative of the compound of Formula (38) or its stereoisomer, or a salt of any of the foregoing, to produce the compound of Formula (34), or a stereoisomer thereof, or a deuterated derivative of the compound of Formula (34) or its stereoisomer, or a salt of any of the foregoing,
wherein:
   R^{a} is selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or NR^{a}₂ is NO₂; and
   R^{b} is selected from benzyl (Bn), naphthylmethyl (Nap), biphenylmethyl, Ac, TFA, and Bz.
202. The method according to Embodiment 201, wherein R^{a} is Boc and R^{b} is Bn.
202a. The method according to Embodiment 201a, wherein NR^{a}₂ is NO₂.
203. The method according to any one of Embodiments 201 to 202a, wherein combining the compound of Formula (37), or a deuterated derivative or salt thereof, with the compound of Formula (38), or a stereoisomer thereof, or a deuterated derivative of the compound of Formula (38) or its stereoisomer, or a salt of any of the foregoing, is performed in the presence of a peptide coupling agent, or wherein combining the compound of Formula (37a), or a deuterated derivative or salt thereof, with the compound of Formula (38), or a stereoisomer thereof, or a deuterated derivative of the compound of Formula (38) or its stereoisomer, or a salt of any of the foregoing, is performed in the presence of a peptide coupling agent.
204. The method according to Embodiment 203, wherein the peptide coupling agent is 1,1′-carbonyldiimidazole (CDI).
205. The method according to Embodiment 201, wherein the compound of Formula (37):

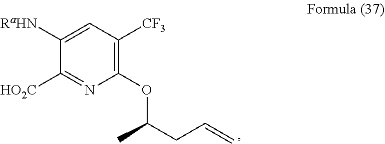

or a deuterated derivative or salt thereof, is prepared by converting a compound of Formula (39):

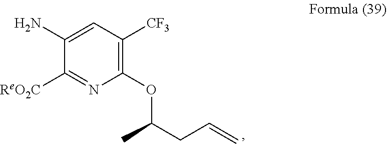

or a deuterated derivative or salt thereof, into the compound of Formula (37), or a deuterated derivative or salt thereof,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{e} is selected from Me, Et, n-Pr, i-Pr, and t-Bu.
205a. The method according to Embodiment 201a, wherein the compound of Formula (37a):

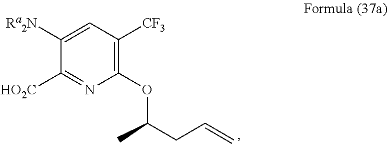

or a deuterated derivative or salt thereof, is prepared by converting a compound of Formula (39a):

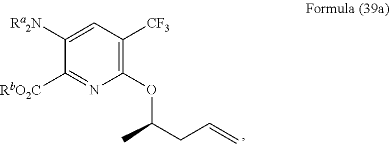

or a deuterated derivative or salt thereof, into the compound of Formula (37a), or a deuterated derivative or salt thereof,
wherein:
   R^{a} is selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or NR^{a}₂ is NO₂; and
   R^{e} is selected from Me, Et, n-Pr, i-Pr, and t-Bu.
206. The method according to Embodiment 205, wherein R^{a} is Boc and R^{e} is Me.
206a. The method according to Embodiment 205a, wherein NR^{a}₂ is NO₂ and R^{e} is Me.
207. The method according to Embodiment 205 or 206, wherein converting the compound of Formula (39), or a deuterated derivative or salt thereof, into the compound of Formula (37), or a deuterated derivative or salt thereof, is performed in the presence of di-tert-butyl dicarbonate (Boc₂O) and 4-dimethylaminopyridine (DMAP).
207a. The method according to Embodiment 205a or 206a, wherein converting the compound of Formula (39a), or a deuterated derivative or salt thereof, into the compound of Formula (37a), or a deuterated derivative or salt thereof, is performed in the presence of lithium iodide.
208. The method according to Embodiment 205, wherein converting the compound of Formula (39), or a deuterated derivative or salt thereof, into the compound of Formula (37), or a deuterated derivative or salt thereof, comprises the following steps:
   a) converting the compound of Formula (39):

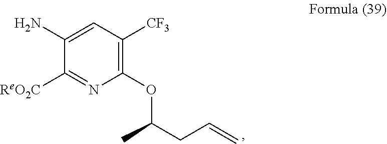

or a deuterated derivative or salt thereof, into the compound of Formula (40):

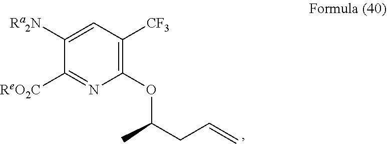

or a deuterated derivative thereof; and
   b) converting the compound of Formula (40), or a deuterated derivative thereof, into the compound of Formula (37), or a deuterated derivative or salt thereof,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{e} is selected from Me, Et, n-Pr, i-Pr, and t-Bu.
209. The method according to Embodiment 208, wherein R^{a} is Boc and R^{e} is Me.
210. The method according to Embodiment 208 or 209, wherein step a) is performed in the presence of di-tert-butyl dicarbonate (Boc₂O).
211. The method according to Embodiment 208 or 209, wherein step b) is performed in the presence of an aqueous hydroxide base.
212. The method according to Embodiment 211, wherein the aqueous hydroxide base is selected from aqueous lithium hydroxide (LiOH), aqueous sodium hydroxide (NaOH), and aqueous potassium hydroxide (KOH).
213. The method according to Embodiment 211 or 212, wherein the aqueous hydroxide base is aqueous lithium hydroxide (LiOH).
214. The method according to Embodiment 205, wherein the compound of Formula (39):

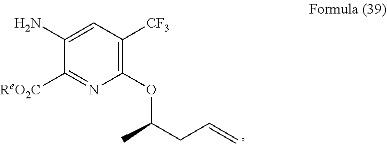

or a deuterated derivative or salt thereof, is prepared by converting a compound of Formula (41):

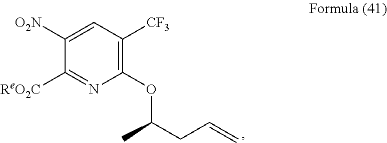

or a deuterated derivative thereof, into the compound of Formula (39), or a deuterated derivative or salt thereof, wherein R^{e} is selected from Me, Et, n-Pr, i-Pr, and t-Bu.
215. The method according to Embodiment 214, wherein R^{e} is Me.
216. The method according to Embodiment 214 or 215, wherein converting the compound of Formula (41), or a deuterated derivative thereof, into the compound of Formula (39), or a deuterated derivative or salt thereof, is performed in the presence of reducing conditions.
217. The method according to Embodiment 216, wherein the reducing conditions are selected from iron (Fe) and acetic acid (AcOH), tin(II) chloride (SnCl₂), and sodium dithionite (Na₂S₂O₄).
218. The method according to Embodiment 216 or 217, wherein the reducing conditions are iron (Fe) and acetic acid (AcOH).
219. The method according to Embodiment 214, wherein the compound of Formula (41):

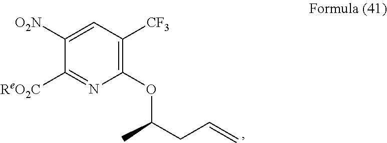

or a deuterated derivative thereof, is prepared by combining a compound of Formula (12):

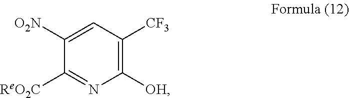

or a deuterated derivative or salt thereof, with compound 2:

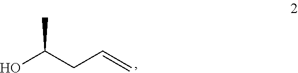

or a deuterated derivative thereof, to produce the compound of Formula (41), or a deuterated derivative thereof, wherein R^{e} is selected from Me, Et, n-Pr, i-Pr, and t-Bu.
220. The method according to Embodiment 219, wherein R^{e} is Me.
221. The method according to Embodiment 219 or 220, wherein combining the compound of Formula (12), or a deuterated derivative thereof, with compound 2, or a deuterated derivative thereof, is performed in the presence of a diazodicarboxylate and a phosphine.
222. The method according to Embodiment 221, wherein the diazodicarboxylate is selected from diisopropyl azodicarboxylate (DIAD), di-2-methoxyethyl azodicarboxylate (DMEAD), di-tert-butyl azodicarboxylate (DTBAD), di-(4-chlorobenzyl)azodicarboxylate (DCAD), 4,4′-azopyridine (AZPY) and 1,1′-(azodicarbonyl)dipiperidine (ADDP).
223. The method according to Embodiment 221 or 222, wherein the phosphine is selected from triphenylphosphine (PPh₃), tris(4-methoxyphenyl)phosphine (P(4-OMe-Ph)₃), tris(4-chlorophenyl)phosphine (P(4-Cl-Ph)₃), tricyclohexylphosphine (PCy₃), methyldiphenylphosphine (PPh₂Me), diphenyl-2-pyridylphosphine (P(2-pyridyl)Ph₂), dicyclohexylphenylphosphine (PCy₂Ph), and 1,2-bis(diphenylphosphino)ethane (dppe).
224. The method according to any one of Embodiments 221 or 224, wherein the diazodicarboxylate is diisopropyl azodicarboxylate (DIAD) and the phosphine is triphenylphosphine (PPh₃).
225. The method according to Embodiment 174 or 181, wherein the compound of Formula (32):

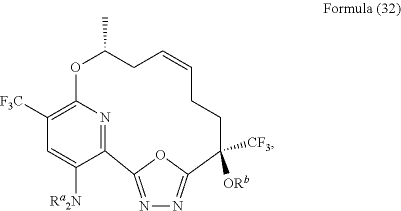

or a stereoisomer thereof, or a deuterated derivative of the compound of Formula (32) or its stereoisomer, or a salt of any of the foregoing, is prepared by converting a compound of Formula (42):

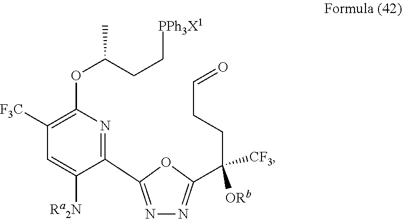

or a stereoisomer thereof, or a deuterated derivative of the compound of Formula (42) or its stereoisomer, or a salt of any of the foregoing, into the compound of Formula (32), or a stereoisomer thereof, or a deuterated derivative of the compound of Formula (32) or its stereoisomer, or a salt of any of the foregoing,
wherein:
   X¹ is selected from Cl, Br, and I;
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
226. The method according to Embodiment 225, wherein X¹ is I, each R^{a} is independently H or Boc, and R^{b} is Bn.
227. The method according to Embodiment 225 or 226, wherein converting the compound of Formula (42), or a deuterated derivative thereof, into the compound of Formula (32), or a stereoisomer thereof, or a deuterated derivative of the compound of Formula (32) or its stereoisomer, or a salt of any of the foregoing, is performed in the presence of sodium tert-butoxide (NaOt-Bu).
228. The method according to Embodiment 225, wherein the compound of Formula (42):

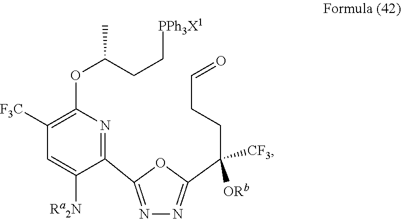

or a stereoisomer thereof, or a deuterated derivative of the compound of Formula (42) or its stereoisomer, or a salt of any of the foregoing, is prepared by converting a compound of Formula (43):

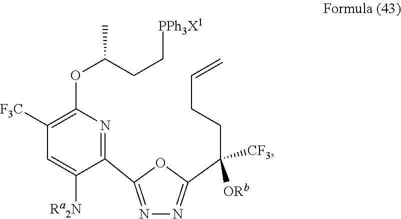

or a stereoisomer thereof, or a deuterated derivative of the compound of Formula (43) or its stereoisomer, or a salt of any of the foregoing, into the compound of Formula (42), or a stereoisomer thereof, or a deuterated derivative of the compound of Formula (42) or its stereoisomer, or a salt of any of the foregoing,
wherein:
   X¹ is selected from Cl, Br, and I;
   each R^{a} is independently selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
229. The method according to Embodiment 228, wherein X¹ is I, each R^{a} is independently H or Boc, and R^{b} is Bn.
230. The method according to Embodiment 228 or 229, wherein converting the compound of Formula (43), or a stereoisomer thereof, or a deuterated derivative of the compound of Formula (43) or its stereoisomer, or a salt of any of the foregoing, into the compound of Formula (42), or a stereoisomer thereof, or a deuterated derivative of the compound of Formula (42) or its stereoisomer, or a salt of any of the foregoing, is performed in the presence of osmium tetroxide (OsO₄) and sodium periodate (NaIO₄).
231. The method according to Embodiment 228, wherein the compound of Formula (43):

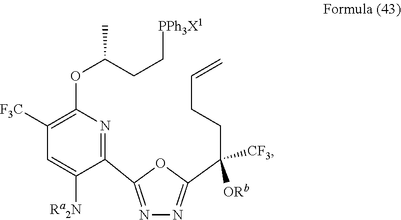

or a stereoisomer thereof, or a deuterated derivative of the compound of Formula (43) or its stereoisomer, or a salt of any of the foregoing, is prepared by combining a compound of Formula (44):

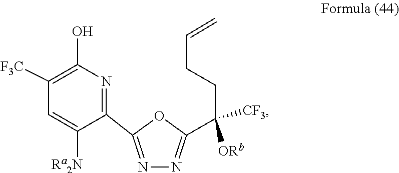

or a stereoisomer thereof, or a deuterated derivative of the compound of Formula (44) or its stereoisomer, or a salt of any of the foregoing, with compound of Formula (45):

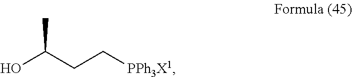

or a deuterated derivative or salt thereof, to produce the compound of Formula (43), or a stereoisomer thereof, or a deuterated derivative of the compound of Formula (43) or its stereoisomer, or a salt of any of the foregoing,
wherein:
   X¹ is selected from Cl, Br, and I;
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
232. The method according to Embodiment 231, wherein X¹ is I, each R^{a} is independently H or Boc and R^{b} is Bn.
233. The method according to Embodiment 231 or 232, wherein combining the compound of Formula (44), or a stereoisomer thereof, or a deuterated derivative of the compound of Formula (44) or its stereoisomer, or a salt of any of the foregoing, with the compound of Formula (45), or a deuterated derivative or salt thereof, is performed in the presence of performed in the presence of a diazodicarboxylate and a phosphine.
234. The method according to Embodiment 233, wherein the diazodicarboxylate is selected from diisopropyl azodicarboxylate (DIAD), di-2-methoxyethyl azodicarboxylate (DMEAD), di-tert-butyl azodicarboxylate (DTBAD), di-(4-chlorobenzyl)azodicarboxylate (DCAD), 4,4′-azopyridine (AZPY) and 1,1′-(azodicarbonyl)dipiperidine (ADDP).
235. The method according to Embodiment 233 or 234, wherein the phosphine is selected from triphenylphosphine (PPh₃), tris(4-methoxyphenyl)phosphine (P(4-OMe-Ph)₃), tris(4-chlorophenyl)phosphine (P(4-Cl-Ph)₃), tricyclohexylphosphine (PCy₃), methyldiphenylphosphine (PPh₂Me), diphenyl-2-pyridylphosphine (P(2-pyridyl)Ph₂), dicyclohexylphenylphosphine (PCy₂Ph), and 1,2-bis(diphenylphosphino)ethane (dppe).
236. The method according to any one of Embodiments 233 to 235, wherein the diazodicarboxylate is diisopropyl azodicarboxylate (DIAD) and the phosphine is triphenylphosphine (PPh₃).
237. The method according to Embodiment 234, wherein the compound of Formula (45):

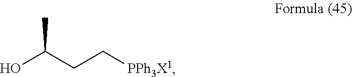

or a deuterated derivative or salt thereof, is prepared by converting a compound of Formula (46):

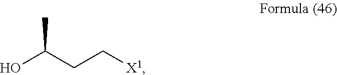

or a deuterated derivative or salt thereof, into the compound of Formula (45), or a deuterated derivative or salt thereof, wherein X¹ is selected from Cl, Br, and I.
238. The method according to Embodiment 237, wherein X¹ is I.
239. The method according to Embodiment 238, wherein converting the compound of Formula (46), or a deuterated derivative or salt thereof, into the compound of Formula (45), or a deuterated derivative or salt thereof, is performed in the presence of triphenylphosphine (PPh₃).
240. The method according to Embodiment 237, wherein the compound of Formula (46):

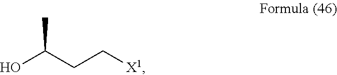

or a deuterated derivative or salt thereof, is prepared by converting (S)-1,3-butanediol (3):

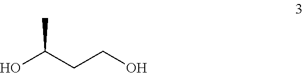

or a deuterated derivative or salt thereof, into the compound of Formula (46), or a deuterated derivative thereof, or a salt of any of the foregoing, wherein X¹ is selected from Cl, Br, and I.
241. The method according to Embodiment 240, wherein converting compound 3, or a deuterated derivative or salt thereof, into the compound of Formula (46), or a deuterated derivative thereof, or a salt of any of the foregoing, is performed in the presence of a halogenating agent, a triphenylphosphine (PPh₃), and 4-dimethylaminopyridine (DMAP).
242. The method according to Embodiment 241, wherein the halogenating agent is selected from bromine (Br₂) and iodine (I₂).
243. The method according to Embodiment 241 or 242, wherein the halogenating agent is iodine (I₂).
244. The method according to Embodiment 231, wherein the compound of Formula (44):

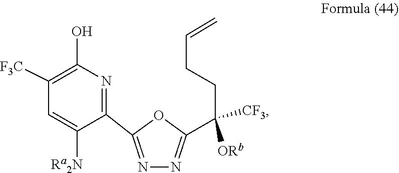

or a stereoisomer thereof, or a deuterated derivative of the compound of Formula (44) or its stereoisomer, or a salt of any of the foregoing, is prepared by converting a compound of Formula (47):

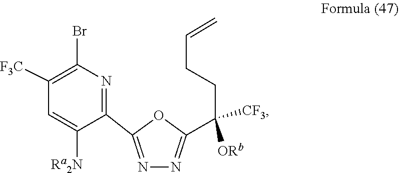

or a stereoisomer thereof, or a deuterated derivative of the compound of Formula (47) or its stereoisomer, or a salt of any of the foregoing, into the compound of Formula (44), or a stereoisomer thereof, or a deuterated derivative of the compound of Formula (44) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
245. The method according to Embodiment 244, wherein each R^{a} is independently H or Boc and R^{b} is Bn.
246. The method according to Embodiment 244 or 245, wherein converting the compound of Formula (47), or a stereoisomer thereof, or a deuterated derivative of the compound of Formula (47) or its stereoisomer, or a salt of any of the foregoing, into the compound of Formula (44), or a stereoisomer thereof, or a deuterated derivative of the compound of Formula (44) or its stereoisomer, or a salt of any of the foregoing, is performed in the presence of cesium acetate (CsOAc).
247. The method according to Embodiment 244, wherein the compound of Formula (47):

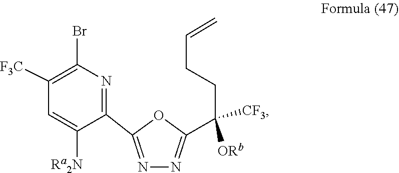

or a stereoisomer thereof, or a deuterated derivative of the compound of Formula (47) or its stereoisomer, or a salt of any of the foregoing, is prepared by converting a compound of Formula (48):

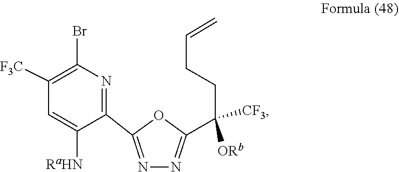

or a stereoisomer thereof, or a deuterated derivative of the compound of Formula (48) or its stereoisomer, or a salt of any of the foregoing, into the compound of Formula (47), or a stereoisomer thereof, or a deuterated derivative of the compound of Formula (47) or its stereoisomer, or a salt of any of the foregoing,
wherein:
   each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
248. The method according to Embodiment 247, wherein each R^{a} is H or Boc and R^{b} is Bn.
249. The method according to Embodiment 247 or 248, wherein converting the compound of Formula (48), or a deuterated derivative thereof, into the compound of Formula (47), or a deuterated derivative thereof, is performed in the presence of di-tert-butyl dicarbonate (Boc₂O), N,N-diisopropylethylamine (DIPEA), and 4-dimethylaminopyridine (DMAP).
250. The method according to Embodiment 247, wherein the compound of Formula (48):

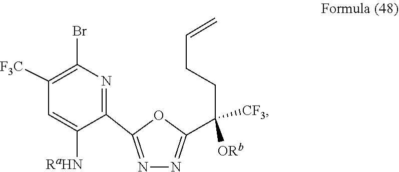

or a stereoisomer thereof, or a deuterated derivative of the compound of Formula (48) or its stereoisomer, or a salt of any of the foregoing, is prepared by converting a compound of Formula (49):

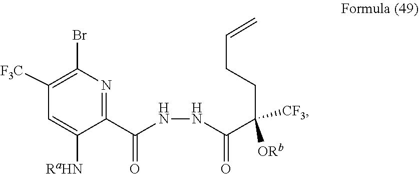

or a stereoisomer thereof, or a deuterated derivative of the compound of Formula (49) or its stereoisomer, or a salt of any of the foregoing, into the compound of Formula (48), or a stereoisomer thereof, or a deuterated derivative of the compound of Formula (48) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
251. The method according to Embodiment 250, wherein R^{a} is Boc and R^{b} is Bn.
252. The method according to Embodiment 250 or 251, wherein converting the compound of Formula (49), or a stereoisomer thereof, or a deuterated derivative of the compound of Formula (49) or its stereoisomer, or a salt of any of the foregoing, into the compound of Formula (48), or a stereoisomer thereof, or a deuterated derivative of the compound of Formula (48) or its stereoisomer, or a salt of any of the foregoing, is performed in the presence of a sulfonyl chloride and an amine base.
253. The method according to claim 252, wherein the sulfonyl chloride is p-toluenesulfonyl chloride (TsCl).
254. The method according to claim 252 or 253, wherein the amine base is N,N-diisopropylethylamine (DIPEA).
255. The method according to any one of claims 252 to 254, wherein the sulfonyl chloride is p-toluenesulfonyl chloride (TsCl) and the amine base is N,N-diisopropylethylamine (DIPEA).
256. The method according to claim 250, wherein the compound of Formula (49):

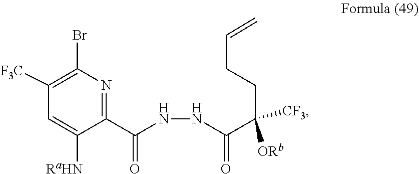

or a stereoisomer thereof, or a deuterated derivative of the compound of Formula (49) or its stereoisomer, or a salt of any of the foregoing, is prepared by combining a compound of Formula (50):

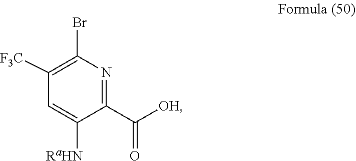

or a deuterated derivative or salt thereof, with the compound of Formula (38):

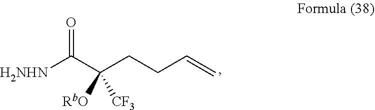

or a stereoisomer thereof, or a deuterated derivative of the compound of Formula (38) or its stereoisomer, or a salt of any of the foregoing, to produce the compound of Formula (49), or a stereoisomer thereof, or a deuterated derivative of the compound of Formula (49) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
257. The method according to Embodiment 256, wherein R^{a} is Boc and R^{b} is Bn.
258. The method according to Embodiment 256 or 257, wherein combining the compound of Formula (50), or a deuterated derivative thereof, or a salt of any of the foregoing, with the compound of Formula (38), or a deuterated derivative thereof, or a salt of any of the foregoing, is performed in the presence of a peptide coupling agent and an amine base.
259. The method according to Embodiment 258, wherein the peptide coupling agent is propylphosphonic anhydride (T3P).
260. The method according to Embodiment 258 or 259, wherein the amine base is N-methylmorpholine (NMM).
261. The method according to any one of Embodiments 258 to 260, wherein the peptide coupling agent is propylphosphonic anhydride (T3P) and the amine base is N-methylmorpholine (NMM).
262. A method of preparing Compound I:

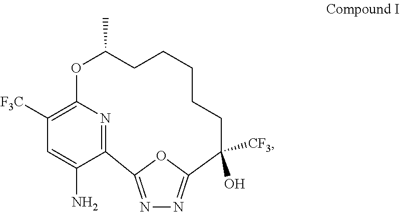 [SMILES: C[C@@H]1CCCCC[C@](O)(C(F)(F)F)C2=NN=C(O2)c2nc(c(C(F)(F)F)cc2N)O1]

or a stereoisomer thereof, or a deuterated derivative of the Compound I or its stereoisomer, or a salt of any of the foregoing, comprising converting a compound of Formula (51):

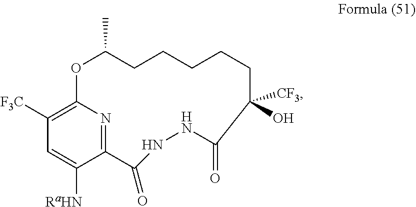

or a stereoisomer thereof, or a deuterated derivative of the compound of Formula (51) or its stereoisomer, or a salt of any of the foregoing, into Compound I, or a stereoisomer thereof, or a deuterated derivative of the Compound I or its stereoisomer, or a salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).
263. The method according to Embodiment 262, wherein R^{a} is Boc.
264. The method according to Embodiment 262, wherein the method comprises converting a compound of Formula (52):

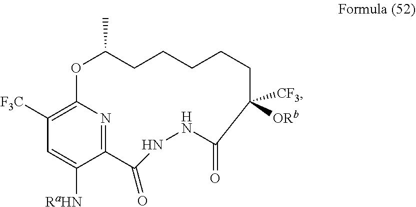

or a stereoisomer thereof, or a deuterated derivative of the compound of Formula (52) or its stereoisomer, or a salt of any of the foregoing, into Compound I, or a stereoisomer thereof, or a deuterated derivative of the Compound I or its stereoisomer, or a salt of any of the foregoing, wherein:
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.
265. The method according to Embodiment 264, wherein R^{a} is Boc and R^{b} is Bn.
266. The method according to Embodiment 264, wherein the compound of Formula (52):

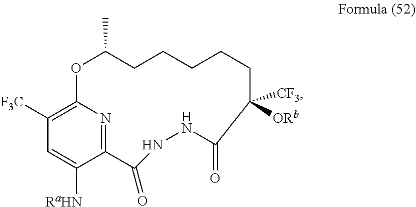

or a stereoisomer thereof, or a deuterated derivative of the compound of Formula (52) or its stereoisomer, or a salt of any of the foregoing, is prepared by converting a compound of Formula (51):

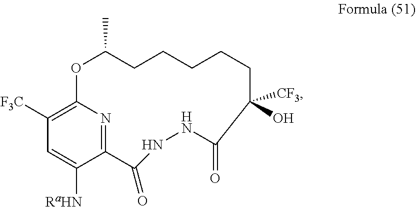

or a stereoisomer thereof, or a deuterated derivative of the compound of Formula (51) or its stereoisomer, or a salt of any of the foregoing, into the compound of Formula (52), or a stereoisomer thereof, or a deuterated derivative of the compound of Formula (52) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
267. The method according to Embodiment 262, wherein the method comprises converting a compound of Formula (3a):

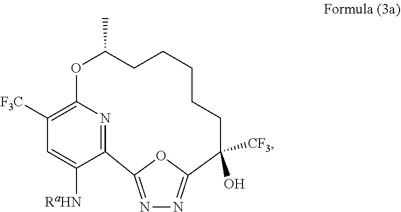

or a stereoisomer thereof, or a deuterated derivative of the compound of Formula (3a) or its stereoisomer, or a salt of any of the foregoing, into Compound I, or a stereoisomer thereof, or a deuterated derivative of the Compound I or its stereoisomer, or a salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).
268. The method according to Embodiment 267, wherein R^{a} is Boc.
269. The method according to Embodiment 267, wherein the compound of Formula (3a):

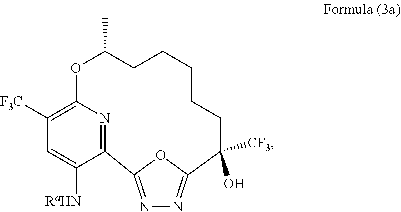

or a stereoisomer thereof, or a deuterated derivative of the compound of Formula (3a) or its stereoisomer, or a salt of any of the foregoing, is prepared by converting a compound of Formula (51):

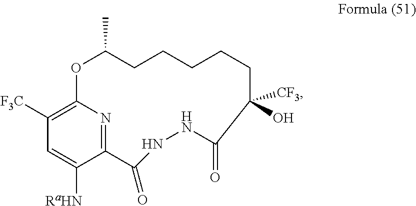

or a stereoisomer thereof, or a deuterated derivative of the compound of Formula (51) or its stereoisomer, or a salt of any of the foregoing, into the compound of Formula (3a), or a stereoisomer thereof, or a deuterated derivative of the compound of Formula (3a) or its stereoisomer, or a salt of any of the foregoing,
wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).
270. The method according to Embodiment 262, wherein the method comprises converting a compound of Formula (2a):

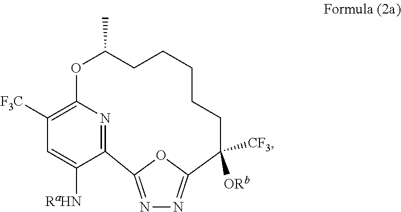

or a stereoisomer thereof, or a deuterated derivative of the compound of Formula (2a) or its stereoisomer, or a salt of any of the foregoing, into Compound I, or a stereoisomer thereof, or a deuterated derivative of the Compound I or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
271. The method according to Embodiment 270, wherein R^{a} is Boc and R^{b} is Bn.
272. The method according to Embodiment 270, wherein the compound of Formula (2a):

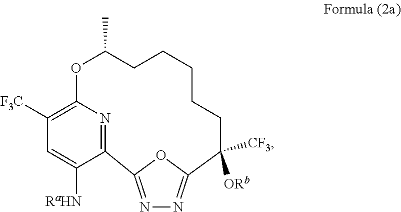

or a stereoisomer thereof, or a deuterated derivative of the compound of Formula (2a) or its stereoisomer, or a salt of any of the foregoing, is prepared by converting a compound of Formula (51):

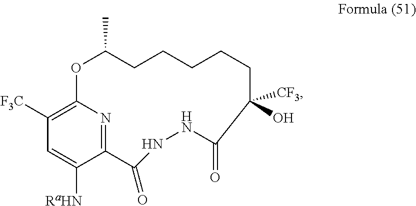

or a stereoisomer thereof, or a deuterated derivative of the compound of Formula (51) or its stereoisomer, or a salt of any of the foregoing, into the compound of Formula (2a), or a stereoisomer thereof, or a deuterated derivative of the compound of Formula (2a) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{b} is selected from benzyl (Bn), naphthylmethyl (Nap), biphenylmethyl, Ac, TFA, and Bz.
273. The method according to Embodiment 262, wherein the compound of Formula (51):

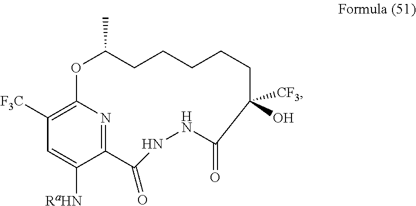

or a stereoisomer thereof, or a deuterated derivative of the compound of Formula (51) or its stereoisomer, or a salt of any of the foregoing, is prepared by converting a compound of Formula (53):

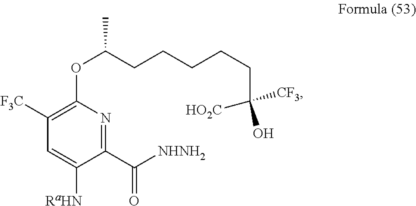

or a stereoisomer thereof, or a deuterated derivative of the compound of Formula (53) or its stereoisomer, or a salt of any of the foregoing, into the compound of Formula (51), or a stereoisomer thereof, or a deuterated derivative of the compound of Formula (51) or its stereoisomer, or a salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).
274. The method according to Embodiment 273, wherein R^{a} is Boc.
275. The method according to Embodiment 273, wherein converting the compound of Formula (53), or a stereoisomer thereof, or a deuterated derivative of the compound of Formula (53) or its stereoisomer, or a salt of any of the foregoing, into the compound of Formula (51), or a stereoisomer thereof, or a deuterated derivative of the compound of Formula (51) or its stereoisomer, or a salt of any of the foregoing, is performed peptide coupling agent and an amine base.
276. The method according to Embodiment 275, wherein the peptide coupling agent is 1-[bis(dimethylamino)methylene]-1H-1,2,3-triazolo[4,5-b]pyridinium 3-oxid hexafluorophosphate (HATU).
277. The method according to Embodiment 275 or 276, wherein the amine base is N,N-diisopropylethylamine (DIPEA).
278. The method according to any one of Embodiments 275 to 277, wherein the peptide coupling agent is 1-[bis(dimethylamino)methylene]-1H-1,2,3-triazolo[4,5-b]pyridinium 3-oxid hexafluorophosphate (HATU) and the amine base is N,N-diisopropylethylamine (DIPEA).
279. The method according to Embodiment 273, wherein the compound of Formula (53):

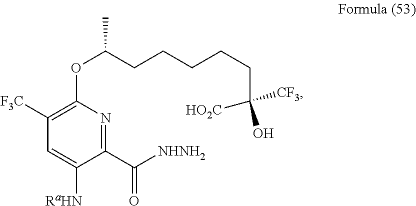

or a stereoisomer thereof, or a deuterated derivative of the compound of Formula (53) or its stereoisomer, or a salt of any of the foregoing, is prepared by converting a compound of Formula (54):

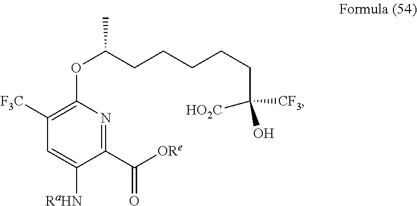

or a stereoisomer thereof, or a deuterated derivative of the compound of Formula (54) or its stereoisomer, or a salt of any of the foregoing, into the compound of Formula (53), or a stereoisomer thereof, or a deuterated derivative of the compound of Formula (53) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{e} is selected from Me, Et, n-Pr, i-Pr, and t-Bu.
280. The method according to Embodiment 279, wherein R^{a} is Boc and R^{e} is Me.
281. The method according to Embodiment 279 and 280, wherein converting the compound of Formula (54), or a stereoisomer thereof, or a deuterated derivative of the compound of Formula (54) or its stereoisomer, or a salt of any of the foregoing, is performed in the presence of hydrazine source and optionally an additive.
282. The method according to Embodiment 281, wherein the hydrazine source is selected from hydrazine hydrate, hydrazine monohydrochloride, hydrazine dihydrochloride, and hydrazine sulfate salt.
283. The method according to Embodiment 281 or 282, wherein the additive is selected from guanidine bases.
284. The method according to any one of Embodiments 281 to 283, wherein the additive is 1,5,7-triazabicyclo[4.4.0]dec-5-ene (TBD).
285. The method according to any one of Embodiments 281 to 284, wherein the hydrazine source is hydrazine hydrate and the additive is 1,5,7-triazabicyclo[4.4.0]dec-5-ene (TBD).
286. The method according to Embodiment 279, wherein the compound of Formula (54):

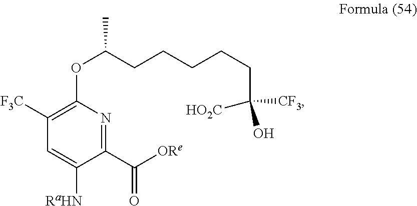

or a stereoisomer thereof, or a deuterated derivative of the compound of Formula (54) or its stereoisomer, or a salt of any of the foregoing, is prepared by converting a compound of Formula (55):

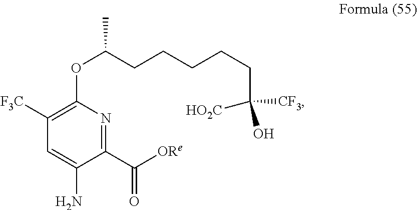

or a stereoisomer thereof, or a deuterated derivative of the compound of Formula (55) or its stereoisomer, or a salt of any of the foregoing, into the compound of Formula (54), or a stereoisomer thereof, or a deuterated derivative of the compound of Formula (54) or its stereoisomer, or a salt of any of the foregoing,
wherein:
   R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
   R^{e} is selected from Me, Et, n-Pr, i-Pr, and t-Bu.
287. The method according to Embodiment 286, wherein R^{a} is Boc and R^{e} is Me.
288. The method according to Embodiment 286 or 287, wherein converting the compound of Formula (55), or a stereoisomer thereof, or a deuterated derivative of the compound of Formula (55) or its stereoisomer, or a salt of any of the foregoing, into the compound of Formula (54), or a stereoisomer thereof, or a deuterated derivative of the compound of Formula (54) or its stereoisomer, or a salt of any of the foregoing, is performed in the presence of di-tert-butyl dicarbonate (Boc₂O), N-methylmorpholine (NMM), and 4-dimethylaminopyridine (DMAP).
289. The method according to Embodiment 286, wherein the compound of Formula (55):

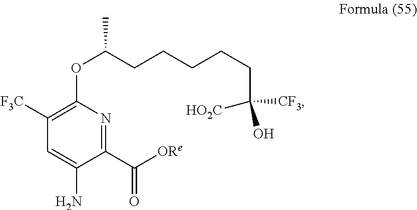

or a stereoisomer thereof, or a deuterated derivative of the compound of Formula (55) or its stereoisomer, or a salt of any of the foregoing, is prepared by converting a compound of Formula (56):

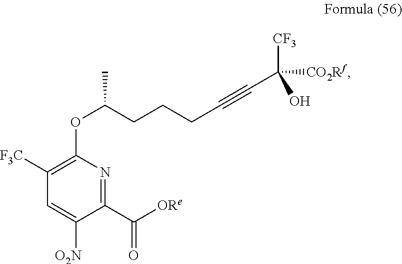

or a stereoisomer thereof, or a deuterated derivative of the compound of Formula (56) or its stereoisomer, or a salt of any of the foregoing, into the compound of Formula (55), or a stereoisomer thereof, or a deuterated derivative of the compound of Formula (55) or its stereoisomer, or a salt of any of the foregoing,
wherein:
   R^{e} is selected from Me, Et, n-Pr, i-Pr, and t-Bu.
   R^{f} is selected from Me, Et, and Bn.
290. The method according to Embodiment 289, wherein R^{e} is Me and R^{f} is Bn.
291. The method according to Embodiment 289 or 290, wherein converting the compound of Formula (56), or a stereoisomer thereof, or a deuterated derivative of the compound of Formula (56) or its stereoisomer, or a salt of any of the foregoing, into the compound of Formula (55), or a stereoisomer thereof, or a deuterated derivative of the compound of Formula (55) or its stereoisomer, or a salt of any of the foregoing, is performed in the presence of hydrogen gas (H₂) and a palladium catalyst.
292. The method according to Embodiment 291, wherein the palladium catalyst is selected from palladium on carbon (Pd/C) and palladium on alumina (Pd/Al).
293. The method according to Embodiment 291 or 292, wherein the palladium catalyst is palladium on carbon (Pd/C).
294. The method according to Embodiment 289, wherein the compound of Formula (56):

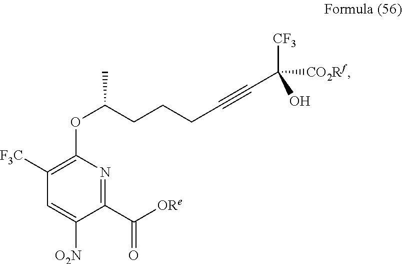

or a stereoisomer thereof, or a deuterated derivative of the compound of Formula (56) or its stereoisomer, or a salt of any of the foregoing, is prepared by combining the compound of Formula (12):

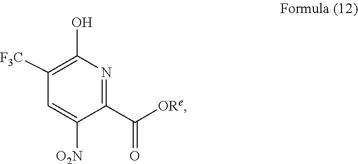

or a deuterated derivative or salt thereof, with a compound of Formula (57):

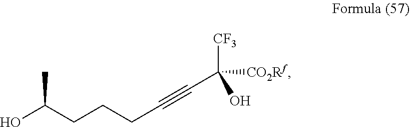

or a stereoisomer thereof, or a deuterated derivative of the compound of Formula (57) or its stereoisomer, or a salt of any of the foregoing, to produce the compound of Formula (56), or a stereoisomer thereof, or a deuterated derivative of the compound of Formula (56) or its stereoisomer, or a salt of any of the foregoing,
wherein:
   R^{e} is selected from Me, Et, n-Pr, i-Pr, and t-Bu.
   R^{f} is selected from Me, Et, and Bn.
295. The method according to Embodiment 294, wherein R^{e} is Me and R^{f} is Bn.
296. The method according to Embodiment 294 or 295, wherein combining the compound of Formula (12), or a deuterated derivative or salt thereof, with the compound of Formula (57), or a stereoisomer thereof, or a deuterated derivative of the compound of Formula (57) or its stereoisomer, or a salt of any of the foregoing, is performed in the presence of performed in the presence of a diazodicarboxylate, a phosphine, and an amine base.
297. The method according to Embodiment 296, wherein the diazodicarboxylate is selected from diisopropyl azodicarboxylate (DIAD), di-2-methoxyethyl azodicarboxylate (DMEAD), di-tert-butyl azodicarboxylate (DTBAD), di-(4-chlorobenzyl)azodicarboxylate (DCAD), 4,4′-azopyridine (AZPY) and 1,1′-(azodicarbonyl)dipiperidine (ADDP).
298. The method according to Embodiment 296 or 297, wherein the phosphine is selected from triphenylphosphine (PPh₃), tris(4-methoxyphenyl)phosphine (P(4—OMe-Ph)₃), tris(4-chlorophenyl)phosphine (P(4-Cl-Ph)₃), tricyclohexylphosphine (PCy₃), methyldiphenylphosphine (PPh₂Me), diphenyl-2-pyridylphosphine (P(2-pyridyl)Ph₂), dicyclohexylphenylphosphine (PCy₂Ph), and 1,2-bis(diphenylphosphino)ethane (dppe).
299. The method according to any one of Embodiments 296 to 298, wherein the amine base is N,N-diisopropylethylamine (DIPEA).
300. The method according to any one of Embodiments 296 to 299, wherein the diazodicarboxylate is diisopropyl azodicarboxylate (DIAD), the phosphine is triphenylphosphine (PPh₃), and the amine base is N,N-diisopropylethylamine (DIPEA).
301. The method according to Embodiment 294, wherein the compound of Formula (57):

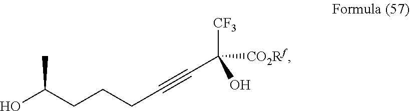

or a stereoisomer thereof, or a deuterated derivative of the compound of Formula (57) or its stereoisomer, or a salt of any of the foregoing, is prepared by converting the compound of Formula (4):

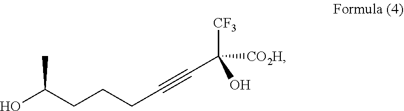 [SMILES: C[C@H](O)CCCC#C[C@@](O)(C(=O)O)C(F)(F)F]

or a stereoisomer thereof, or a deuterated derivative of the compound of Formula (4) or its stereoisomer, or a salt of any of the foregoing, into the compound of Formula (57), or a stereoisomer thereof, or a deuterated derivative of the compound of Formula (57) or its stereoisomer, or a salt of any of the foregoing, wherein R^{f} is selected from Me, Et, and Bn.
302. The method according to Embodiment 301, wherein R^{f} is Bn.
303. The method according to Embodiment 301 or 302, wherein converting the compound of Formula (4), or a stereoisomer thereof, or a deuterated derivative of the compound of Formula (4) or its stereoisomer, or a salt of any of the foregoing, into the compound of Formula (57), or a stereoisomer thereof, or a deuterated derivative of the compound of Formula (57) or its stereoisomer, or a salt of any of the foregoing, is performed in the presence of an alkyl halide, an alkyl triflate, or an alkyl tosylate; and a base.
304. The method according to Embodiment 303, wherein the alkyl halide is a benzyl halide.
305. The method according to Embodiment 303 and 304, wherein the benzyl halide is selected from benzyl chloride (BnCl), benzyl bromide (BnBr), and benzyl iodide (BnI).
306. The method according to any one of Embodiments 303 to 305, wherein the base is selected from lithium carbonate (Li₂CO₃), sodium carbonate (Na₂CO₃), potassium carbonate (K₂CO₃), cesium carbonate (Cs₂CO₃), sodium bicarbonate (NaHCO₃), sodium tert-butoxide (NaOt-Bu), and potassium tert-butoxide (KOt-Bu).
307. The method according to any one of Embodiments 303 to 306, wherein the alkyl halide is benzyl bromide (BnBr) and the base is sodium bicarbonate (NaHCO₃).
308. The method according to Embodiment 301, wherein the compound of Formula (4):

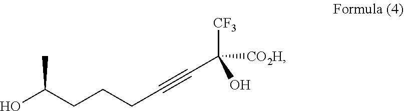 [SMILES: C[C@H](O)CCCC#C[C@@](O)(C(=O)O)C(F)(F)F]

or a stereoisomer thereof, or a deuterated derivative of the compound of Formula (4) or its stereoisomer, or a salt of any of the foregoing, is prepared by converting a compound of Formula (58):

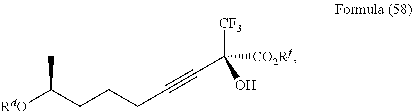

or a stereoisomer thereof, or a deuterated derivative of the compound of Formula (58) or its stereoisomer, or a salt of any of the foregoing, into the compound of Formula (4), or a stereoisomer thereof, or a deuterated derivative of the compound of Formula (4) or its stereoisomer, or a salt of any of the foregoing,
wherein:
   R^{d} is selected from Bz, Bn, pivaloyl (Piv), acetyl (Ac), and t-butyldimethylsilyl (TBDMS); and
   R^{f} is selected from Me, Et, and Bn.
309. The method according to Embodiment 308, wherein R^{d} is Bz and R^{f} is Bn.
310. The method according to Embodiment 308 or 309, wherein converting the compound of Formula (58), or a stereoisomer thereof, or a deuterated derivative of the compound of Formula (58) or its stereoisomer, or a salt of any of the foregoing, into the compound of Formula (4), or a stereoisomer thereof, or a deuterated derivative of the compound of Formula (4) or its stereoisomer, or a salt of any of the foregoing, is performed in the presence of sodium hydroxide (NaOH).
311. The method according to Embodiment 308, wherein the compound of Formula (58):

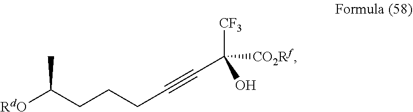

or a stereoisomer thereof, or a deuterated derivative of the compound of Formula (58) or its stereoisomer, or a salt of any of the foregoing, is prepared by combining a compound of Formula (59):

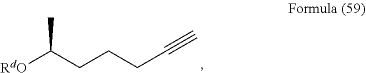

or a deuterated derivative or salt thereof, with a compound of Formula (17),

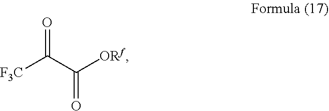

or a deuterated derivative or salt thereof, to produce the compound of Formula (58), or a stereoisomer thereof, or a deuterated derivative of the compound of Formula (58) or its stereoisomer, or a salt of any of the foregoing,
wherein:
   R^{d} is selected from Bz, Bn, pivaloyl (Piv), acetyl (Ac), and t-butyldimethylsilyl (TBDMS); and
   R^{f} is selected from Me, Et, and Bn.
312. The method according to Embodiment 311, wherein R^{d} is Bz and R^{f} is Et.
313. The method according to Embodiment 311 or 312, wherein combining the compound of Formula (59), or a deuterated derivative or salt thereof, with the compound of Formula (17), or a deuterated derivative or salt thereof, is performed in the presence of a rhodium catalyst.
314. The method according to any one of Embodiments 311 to 313, wherein the rhodium catalyst is

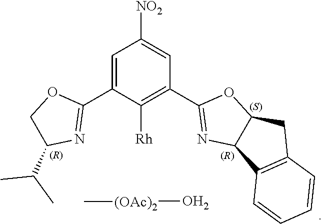

315. The method according to Embodiment 311, wherein the compound of Formula (59):

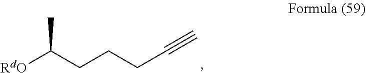

or a deuterated derivative or salt thereof, is prepared by converting compound 5:

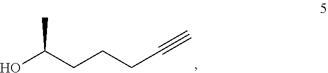

or a deuterated derivative or salt thereof into the compound of Formula (59), or a deuterated derivative or salt thereof, wherein R^{d} is selected from Bz, Bn, pivaloyl (Piv), acetyl (Ac), and t-butyldimethylsilyl (TBDMS).
316. The method according to Embodiment 315, wherein R^{d} is Bz.
317. The method according to Embodiment 315 or 316, wherein converting compound 5, or a deuterated derivative or salt thereof, into the compound of Formula (59), or a deuterated derivative or salt thereof, is performed in the presence of an acid chloride, a base, and optionally an additive.
318. The method according to Embodiment 317, wherein the acid chloride is benzoyl chloride.
319. The method according to Embodiment 317 or 318, wherein the base is triethylamine (TEA).
320. The method according to any one of Embodiments 317 to 319, wherein the additive is 4-dimethylaminopyridine (DMAP).
321. The method according to any one of Embodiments 317 to 320, wherein the acid chloride is benzoyl chloride, the base is triethylamine (TEA), and the additive is 4-dimethylaminopyridine (DMAP).
322. The method according to Embodiment 315, wherein compound 5:

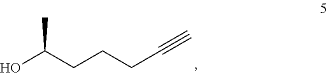

or a deuterated derivative or salt thereof, is prepared by converting compound 6:

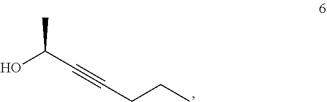

or a deuterated derivative or salt thereof, into compound 5, or a deuterated derivative or salt thereof.
323. The method according to Embodiment 322, wherein converting compound 6, or a deuterated derivative or salt thereof, into compound 5, or a deuterated derivative or salt thereof, is performed in the presence of lithium (Li), potassium tert-butoxide (KOt-Bu), and 1,3-diaminopropane.
324. The method according to Embodiment 322 or 323, wherein compound 6:

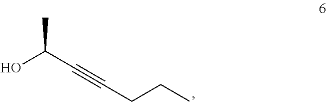

or a deuterated derivative or salt thereof, is prepared by converting compound 7:

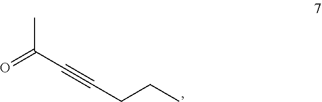

or a deuterated derivative or salt thereof, into compound 6, or a deuterated derivative or salt thereof.
325. The method according to Embodiment 324, wherein converting the compound 7, or a deuterated derivative or salt thereof, into compound 6, or a deuterated derivative or salt thereof, is performed in the presence of (1S,2S)-(+)-N-p-tosyl-1,2-diphenylethylenediamine ((S,S)-TsDPEN), dichloro(mesitylene)ruthenium(II) dimer ([RuCl₂(mesitylene)]2), hexadecyl(trimethyl)ammonium bromide, sodium formate, and water.
326. The method according to Embodiment 324 or 325, wherein compound 7:

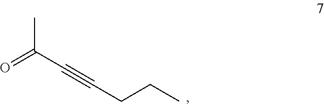

or a deuterated derivative or salt thereof, is prepared by converting compound 8:

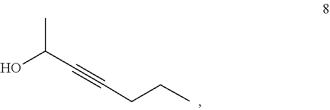

or a deuterated derivative or salt thereof, into compound 7, or a deuterated derivative or salt thereof.
327. The method according to Embodiment 328, wherein converting compound 8, or a deuterated derivative or salt thereof, into compound 7, or a deuterated derivative or salt thereof, is performed in the presence of an oxidant.
328. The method according to Embodiment 327, wherein the oxidant is manganese dioxide (MnO₂).
329. The method according to any one of Embodiments 326 to 328, wherein compound 8:

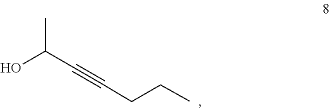

or a deuterated derivative or salt thereof, is prepared by combining compound 9:

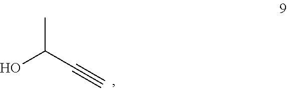

or a deuterated derivative or salt thereof, with a 1-halopropane, or a deuterated derivative thereof, a base, and optionally an additive, to produce compound 8, or a deuterated derivative or salt thereof.
330. The method according to Embodiment 329, wherein the 1-halopropane is selected from 1-chloropropane, 1-bromopropane, and 1-iodopropane.
331. The method according to Embodiment 329 or 330, wherein the base is n-butyllithium (n-BuLi).
332. The method according to any one of Embodiments 329 to 331, wherein the additive is hexamethylphosphoramide (HMPA).
333. The method according to any one of Embodiments 329 to 331, wherein the 1-halopropane is 1-iodopropane, the base is n-butyllithium, and the additive is hexamethylphosphoramide (HMPA).

### EXAMPLES

### Table of Abbreviations

| Abbreviation | Chemical Name |
|---|---|
| AcOH | acetic acid |
| anh. | anhydrous |
| aq. | aqueous |
| AgSbF₆ | silver hexafluoroantimonate(V) |
| AUC | area under curve |
| BnBr | benzyl bromide |
| BnOH | benzyl alcohol |
| BINAP | 2,2′-bis(diphenylphosphino)-1,1′-binapthalene |
| Boc | tert-butyloxycarbonyl |
| Boc—NHNH₂ | tert-butyl carbazate |
| Boc₂O | di-tert-butyl dicarbonate; Boc anhydride |
| BOP | benzotriazol-1-yloxytris(dimethylamino)phosphonium |
| | hexafluorophosphate |
| n-BuLi | n-butyl lithium |
| n-Bu₄NF•H₂O | tetra-n-butylammonium fluoride monohydrate |
| t-BuOH | tert-BuOH; tert-butanol |
| t-BuOK (KOtBu) | potassium tert-butoxide |
| t-BuONa | sodium tert-butoxide |
| Bz-Cl | benzoyl chloride |
| CDCl₃ | chloroform-d |
| CDI | 1,1-carbonyl diimidazole |
| CD₃OD | methyl-d₄ alcohol-d |
| CDMT | 2-chloro-4,6-dimethoxy-1,3,5-triazine |
| CF₃CO₂H | trifluoroacetic acid; perfluoroacetic acid; |
| | trifluoroethanoic acid |
| CH₃CN | acetonitrile |
| CO₂ | carbon dioxide |
| CsOAc | cesium acetate |
| Cs₂CO₃ | cesium carbonate |
| CuI | copper(I) iodide |
| DABCO | 1,4-diazabicyclo[2.2.2]octane |
| DCHA | dicyclohexylamine |
| DCE | 1,2-dichloroethane |
| DCM (CH₂Cl₂) | dichloromethane; methylene chloride |
| DI | deionized |
| DIAD | diisopropyl azodicarboxylate |
| DIEA (DIPEA) | N,N-diisopropylethylamine |
| DMAP | 4-(dimethylamino)pyridine |
| DMF | N,N-dimethylformamide |
| DMP | Dess-Martin periodinane |
| DMSO | dimethyl sulfoxide |
| DMSO-d6 | dimethyl sulfoxide-d6 |
| EDCI | 1-ethyl-3-(3-dimethylaminopropyl)carbodiimide |
| ELSD | evaporative light scattering detector |
| ESI-MS | electrospray ionization mass spectrometry |
| Et₂O | diethyl ether |
| Et₃N (TEA) | triethylamine |
| EtOAc | ethyl acetate |
| EtOH | ethanol |
| FIH | first in human |
| Fe | iron |
| ¹⁹F NMR | fluorine nuclear magnetic resonance |
| Grubbs catalyst 2^{nd} | [1,3-bis(2,4,6-trimethylphenyl)imidazolidin-2- |
| generation | ylidene]-dichloro-[(2- |
| | isopropoxyphenyl)methylene]ruthenium |
| H₂ | hydrogen gas |
| H₂O | water |
| HATU | 1-[bis(dimethylamino)methylene]-1H-1,2,3- |
| | triazolo[4,5-b]pyridinium 3-oxide |
| | hexafluorophosphate |
| HCl | hydrochloric acid |
| HCN | hydrogen cyanide |
| HPLC | high pressure liquid chromatography |
| HMPA | hexamethylphosphoramide |
| ¹H NMR | proton nuclear magnetic resonance |
| HNL | hydroxynitrile lyase enzyme |
| HNO₃ | nitric acid |
| HOAc (AcOH) | acetic acid |
| HOBT | hydroxy benzotriazole |
| HPLC | high performance liquid chromatography |
| H₂O | water |
| H₂O₂ | hydrogen peroxide |
| H₂SO₄ | sulfuric acid |
| I₂ | iodine |
| IPAc (iPrOAc) | isopropyl acetate |
| K₂CO₃ | potassium carbonate |
| KHCO₃ | potassium bicarbonate |
| KHSO₄ | potassium bisulfate |
| KMnO₄ | potassium permanganate |
| KOH | potassium hydroxide |
| K₃PO₄ | potassium phosphate tribasic |
| KRED | ketoreductase |
| LC | liquid chromatography |
| LCMS | liquid chromatography mass spectrometry |
| Li | lithium metal |
| LiAlH₄ | lithium aluminum hydride |
| LiOH | lithium hydroxide |
| MeOH | methanol |
| MeCN (CH₃CN; ACN) | acetonitrile |
| MeMgCl | methyl magnesium chloride |
| 2-MeTHF | 2-methyltetrahydrofuran |
| MgSO₄ | magnesium sulfate |
| MnO₂ | manganese dioxide |
| MS | mass spectrometry |
| MTBE | methyl tert-butyl ether |
| NH₄HCO₂ | ammonium formate |
| Na | sodium |
| NaCl | sodium chloride |
| NaH: | sodium hydride |
| NaHCO₃ | sodium bicarbonate |
| NaIO₄ | sodium periodate |
| NaSMe | sodium thiomethoxide (sodium methanethiolate) |
| NaOAc | sodium acetate |
| NaOH | sodium hydroxide |
| Na₂S₂O₄ | sodium dithionite |
| Na₂S₂O₃ | sodium thiosulfate |
| Na₂SO₄ | sodium sulfate |
| NBS | N-bromosuccinimide |
| NH₃ | ammonia |
| NH₄Cl | ammonium chloride |
| NH₄HCO₃ | ammonium bicarbonate |
| NLT | no less then |
| NMM | N-methylmorpholine, 4-methylmorpholine |
| NMP | N-methyl-2-pyrrolidone |
| NMR | nuclear magnetic resonance |
| N₂ | dinitrogen |
| MTBE | methyl tert-butyl ether |
| OsO₄ | osmium tetroxide |
| Pd/C | palladium on carbon |
| PdCl₂ | palladium(II) chloride |
| PdCl₂(S-BINAP) | dichloro[2,2′-bis(diphenylphosphino)-1,1′- |
| | binaphthyl]palladium(II) |
| Pd₂(dba)₃ | tris(dibenzylideneacetone)dipalladium(0) |
| Pd(dppf)Cl₂ | 1,1′-bis(diphenylphosphino)ferrocene palladium(II) |
| | chloride |
| Pd(OAc)₂ | palladium (II) acetate |
| PE:EA | petroleum ether:ethyl acetate |
| PhI(OAc)₂ | (diacetoxyiodo)benzene |
| PhMe | toluene |
| PPh₃ | triphenylphosphine |
| POCl₃ | phosphoryl chloride |
| PtO₂ | platinum oxide |
| QNMR | quantitative nuclear magnetic resonance |
| RBF | round bottom flask |
| RCM | ring closing metathesis |
| RT | room temperature |
| RuCl₃ | ruthenium(III) chloride |
| RuCl₂(mesitylene) | dichloro(mesitylene)ruthenium(II) |
| SFC | supercritical fluid chromatography |
| Silica Cat Pd | palladium on silica |
| SiO₂ | silicon dioxide; silica; silica gel |
| SM | starting material |
| TBAF | tetrabutylammonium fluoride |
| TBAI | tetrabutylammonium iodide |
| TBD | 1,5,7-Triazabicyclo[4.4.0]dec-5-ene |
| TBDPS | tert-butyldiphenylsilyl |
| TBDPSiCl (TBDPS-Cl or | tert-butyl(chloro)diphenylsilane |
| TBDPSCl) | |
| TEA (Et₃N) | triethylamine |
| TFA | trifluoroacetic acid |
| TFAA | trifluoroacetic anhydride |
| THF | tetrahydrofuran |
| Ti(OEt)₄ | titanium (IV)ethoxide |
| Tⱼ | temperature of jacket |
| TLC | thin layer chromatography |
| TMEDA | tetramethylethylenediamine |
| TMSCF₃ | trifluoromethyltrimethylsilane |
| Tᵣ | temperature of reactor |
| T3P (PPAA) | propanephosphonic acid anhydride |
| TPPO (Ph₃PO) | triphenylphospine oxide |
| p-TsCl; TsCl | p-toluenesulfonyl chloride; tosyl chloride |
| (S,S)-TsDPEN | (1S,2S)-N-(p-toluenesulfony1)-1,2- |
| | diphenylethanediamine |
| UHP | urea hydrogen peroxide |
| Umicore M101 Ru- | Grubbs Catalyst ® M101 (Sigma Aldrich Cat. No. |
| Catalyst | 774901): dichloro(3-phenyl-1H-inden-1- |
| | ylidene)bis(tricyclohexylphosphine)ruthenium(II) |
| UPLC | ultra performance liquid chromatography |
| w/v | weight by volume |
| w/w | weight by weight |
| wt | weight |
| XantPhos | 4,5-bis(diphenylphosphino)-9,9-dimethylxanthene |
| XantPhos Pd G4 | (6-diphenylphosphanyl-10H-phenoxazin-4-yl)- |
| | diphenylphosphane; methanesulfonic acid; N-methyl- |
| | 2-phenylaniline; palladium (Sigma Aldrich Catalog |
| | No. 900329) |
| Zhan catalyst-1B | dichloro[1,3-bis(2,4,6-trimethylphenyl)-2- |
| | imidazolidinylidene][[5-[(dimethylamino)sulfonyl]-2- |
| | (1-methylethoxy-O)phenyl]methylene-C]ruthenium(II) |

### General Solid State NMR (SSNMR) Method

A Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C cross-polarization (CP) MAS experiment. The fluorine relaxation time was measured using ¹⁹F MAS Ti saturation recovery relaxation experiment in order to set up proper recycle delay of the ¹⁹F MAS experiment. The CP contact time of carbon CPMAS experiment was set to 2 ins. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### General X-Ray Powder Diffraction (XRPD) Method

The X-ray powder diffraction (XRPD) pattern was recorded at room temperature in continuous mode using a PANalytical Empyrean X-ray Diffract meter (Almelo, The Netherlands). The X-ray was generated using Cu tube operated at 45 kV and 40 mA. Pixel Id detector was used with anti-scatter slit P8. The Divergence optics was Bragg Brentano High Definition (BBHD) with a 10 mm mask, ⅛ divergence slit, and ½ anti-scatter slit. The continuous scan mode utilized a 0.0131 degree step size and count time of 13.77 seconds per step, integrated over the range from 4 to 40 degrees two-theta. The powder sample was placed on an indented area within a zero background holder and flattened with a glass slide.

### General Thermogravimetric Analysis (TGA) Method

TGA was used to investigate the presence of residual solvents in the lots characterized and identify the temperature at which decomposition of the sample occurs. Unless provided otherwise in the following Examples, TGA data were collected on a Mettler Toledo TGA/DSC 3+ STARe System. TGA data for Compound 4 were collected on a TA instrument Discovery series with TRIOS system.

### General Differential Scanning Calorimetry (DSC) Method

Unless provided otherwise in the following Examples, the melting point or glass transition point of the material was measured using a Mettler Toledo TGA/DSC 3+ STARe System. DSC data for Compound 4 were collected on a TA instrument Discovery series with TRIOS system.

### Example 1: Preparation of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol (Compound I)

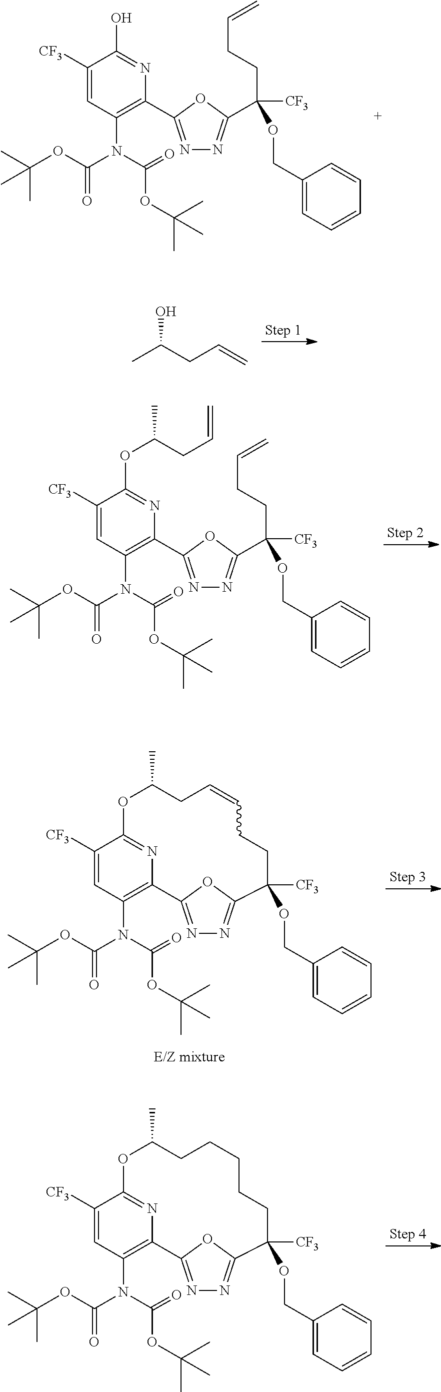

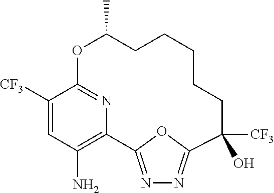 [SMILES: C[C@@H]1CCCCC[C@](O)(C(F)(F)F)C2=NN=C(O2)c2nc(c(C(F)(F)F)cc2N)O1]

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

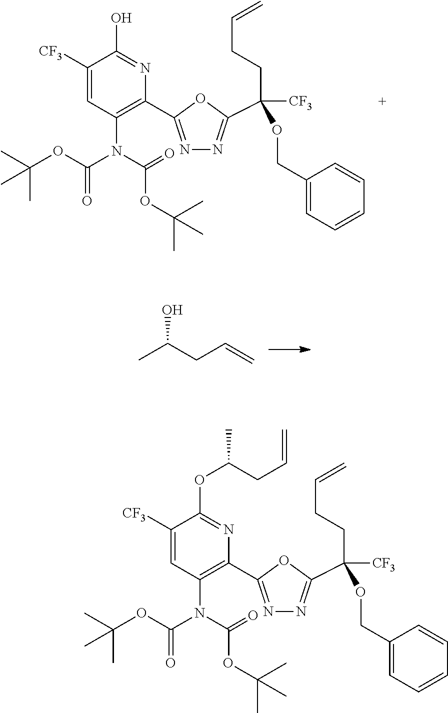

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 min. For the next approximately 2 h, the mixture was cooled to room temperature. During this cooling period, after the first 10 minutes, triphenylphosphine (6.07 g, 23.14 mmol) was added. After a further 1 h, additional triphenylphosphine (3.04 g, 11.59 mmol) was added. After a further 23 min, DIAD (2.24 mL, 11.57 mmol) was added. After the ˜2 h cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 min, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m/z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture)

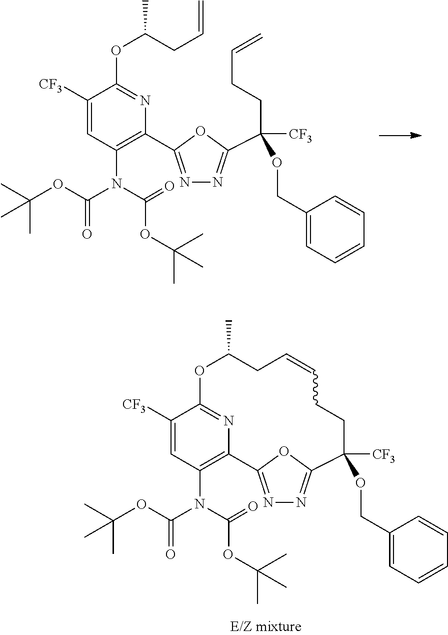 [SMILES: C=CCC[C@@](OCc1ccccc1)(c1nnc(-c2nc(O[C@H](C)CC=C)c(C(F)(F)F)cc2N(C(=O)OC(C)(C)C)C(=O)OC(C)(C)C)o1)C(F)(F)F ; C[C@@H]1CC=CCC[C@](OCc2ccccc2)(C(F)(F)F)C2=NN=C(O2)c2nc(c(C(F)(F)F)cc2N(C(=O)OC(C)(C)C)C(=O)OC(C)(C)C)O1]

The following reaction was run, split equally between two, 12 L reaction flasks run in parallel. Mechanical stirring was employed, and reactions were subjected to a constant nitrogen gas purge using a course porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st} Generation Catalyst (9 g, 10.94 mmol in each flask) was added to each reaction and stirred at 400 rpm while setting an internal temperature control to 75° C. with strong nitrogen purging (both reactions reached ˜75° C. after approximately 20 min). After 5 h 15 min, the internal temperature control was set to 45° C. After approximately 2 h, 2-sulfanylpyridine-3-carboxylic acid (11 g, 70.89 mmol in each flask) was added to each flask followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 h, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg C₁₈ column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m/z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

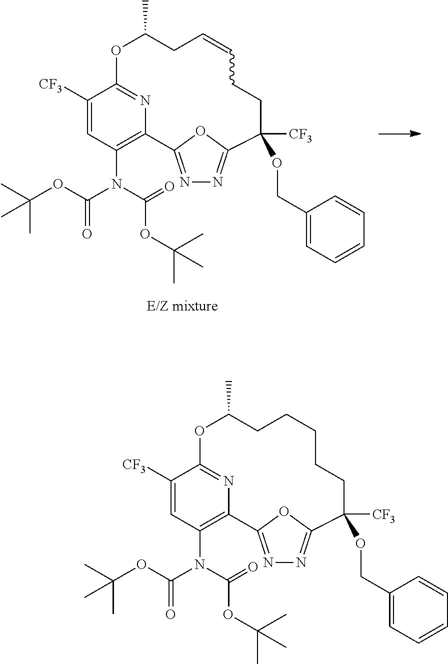 [SMILES: C[C@@H]1CC=CCC[C@](OCc2ccccc2)(C(F)(F)F)C2=NN=C(O2)c2nc(c(C(F)(F)F)cc2N(C(=O)OC(C)(C)C)C(=O)OC(C)(C)C)O1 ; C[C@@H]1CCCCC[C@](OCc2ccccc2)(C(F)(F)F)C2=NN=C(O2)c2nc(c(C(F)(F)F)cc2N(C(=O)OC(C)(C)C)C(=O)OC(C)(C)C)O1]

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 h. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL) charged with fresh 10% Pd/C (50% water wet, 2.2 g of 5% w/w, 1.034 mmol) and stirred vigorously under hydrogen (balloon) for 6 h, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m/z calc. 730.2801, found 731.0 (M+1)+; Retention time: 3.89 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol, Compound I

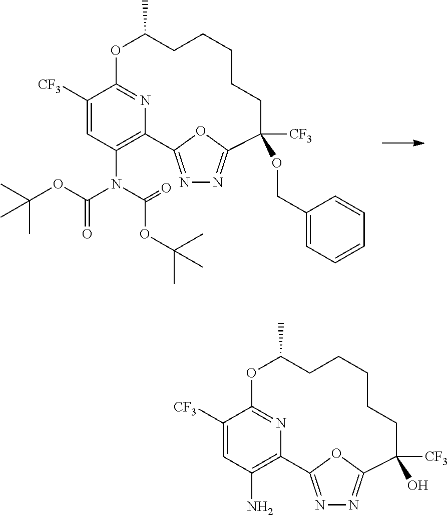 [SMILES: C[C@@H]1CCCCC[C@](OCc2ccccc2)(C(F)(F)F)C2=NN=C(O2)c2nc(c(C(F)(F)F)cc2N(C(=O)OC(C)(C)C)C(=O)OC(C)(C)C)O1 ; C[C@@H]1CCCCC[C@](O)(C(F)(F)F)C2=NN=C(O2)c2nc(c(C(F)(F)F)cc2N)O1]

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5% w/w, 0.8457 mmol) then cycled 3 times between vacuum/nitrogen and 3 times between vacuum/hydrogen and then stirred vigorously under hydrogen (balloon) at room temperature for 18 h. The mixture was cycled 3 times between vacuum/nitrogen, filtered over Celite washing with ethanol and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5]nonadeca-1(18),2,4,14,16-pentaen-17-yl]carbamate an off-white solid. This material was dissolved in dichloromethane (69 mL), cooled in an ice bath under nitrogen and slowly treated with TFA (23 mL, 298.5 mmol). The solution was stirred in the ice bath for 5 min and then at room temperature for 1 h. The pale-yellow solution was diluted with heptane (˜100 mL) and evaporated to give a yellow solid mass. The residue was diluted again with heptane (˜100-200 mL) and dichloromethane was added under warming until a yellow solution was obtained. Most of the dichloromethane was removed by rotary evaporation (35° C. water bath, 100 mbar pressure) to give a fine yellow suspension. The suspension was swirled for ˜1 h at room temperature, filtered washing the solid with dry ice chilled heptane and then dried over 3 days under vacuum with a nitrogen leak at 50° C. to give as a pale yellow solid, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol (4.68 g, 90%). ¹H NMR (400 MHz, DMSO-d₆) δ 7.77 (s, 1H), 7.55 (s, 1H), 6.34 (s, 2H), 4.90-4.70 (m, 1H), 2.47 (dd, J=7.8, 5.5 Hz, 1H), 2.29 (t, J=11.2 Hz, 1H), 2.11 (ddd, J=14.4, 8.7, 6.1 Hz, 1H), 1.73 (dt, J=12.7, 7.6 Hz, 2H), 1.59-1.38 (m, 4H), 1.35 (d, J=6.3 Hz, 3H), 1.18 (ddt, J=12.4, 9.6, 6.2 Hz, 1H) ppm. ¹H NMR (400 MHz, Chloroform-d) δ 7.42 (d, J=0.8 Hz, 1H), 5.20 (s, 2H), 4.75 (dtt, J=12.6, 6.3, 3.2 Hz, 1H), 3.98 (s, 1H), 2.68 (dtd, J=12.9, 7.6, 2.3 Hz, 1H), 2.38-2.18 (m, 2H), 2.03 (d, J=7.9 Hz, 1H), 1.75-1.46 (m, 5H), 1.41 (d, J=6.3 Hz, 3H), 1.35-1.27 (m, 1H) ppm. ¹⁹F NMR (376 MHz, Chloroform-d) δ −63.95, −77.34 ppm. ESI-MS m/z calc. 440.1283, found 441.0 (M+1)⁺; Retention time: 2.87 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 5: Solid Form Chracterization of Crystalline Compound I Heptane Solvate Form A

### A. X-Ray Powder Diffraction

The X-ray powder diffraction (XRPD) diffractogram of the product of Step 4, crystalline Compound I heptane solvate Form A, was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 2θ with a step size of 0.01313030 and 49 s per step.

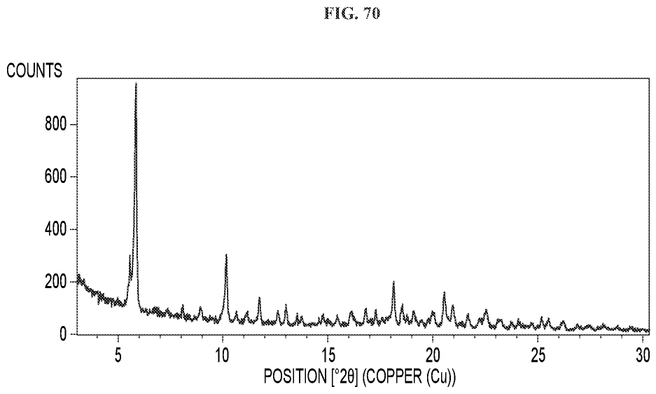

The XRPD diffractogram for crystalline Compound I heptane solvate Form A is provided in FIG. 70, and the XRPD data are summarized below in Table 2.

TABLE 2
XRPD Signals for Crystalline Compound
I Heptane Solvate Form A

| XRPD Peak No. | Angle (degrees 2-Theta ± 0.2) | Intensity % |
|---|---|---|
| 1 | 5.8 | 100.0 |
| 2 | 10.1 | 30.5 |
| 3 | 5.6 | 22.3 |
| 4 | 18.1 | 21.0 |
| 5 | 20.5 | 15.8 |
| 6 | 11.7 | 12.4 |
| 7 | 20.9 | 11.3 |

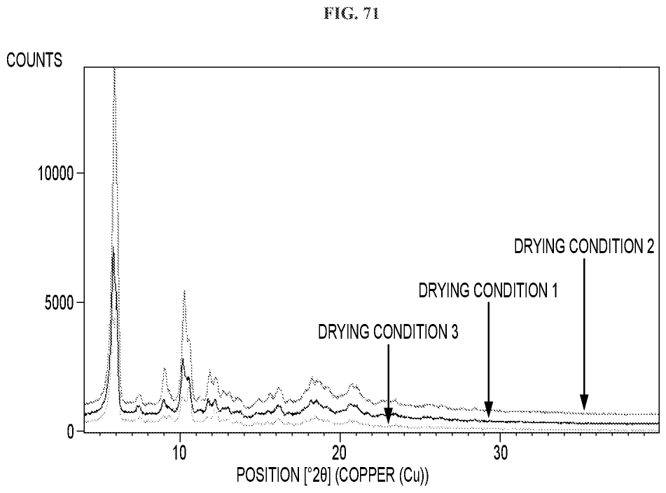

XRPD diffractograms for crystalline Compound I heptane solvate Form A samples prepared under three different drying conditions are provided in FIG. 71. The XRPD diffractograms were recorded at room temperature in continuous mode using a PANalytical Empyrean X-ray Diffract meter (Almelo, The Netherlands). The X-Ray was generated using Cu tube operated at 45 kV and 40 mA. Pixel 1d detector was used with anti-scatter slit P8. The Divergence optics is Bragg Brentano High Definition (BBHD) with a 10 mm mask, ⅛ divergence slit, and ½ anti-scatter slit. The continuous scan mode utilized a 0.0131 degree step size and count time of 13.77 seconds per step, integrated over the range from 4 to 40 degrees two-theta. The powder sample was placed on an indented area within a zero background holder and flattened with a glass slide.

Under Drying Condition 1, crystalline Compound I heptane solvate Form A was dried over the weekend under house vacuum with a nitrogen leak at 50° C. Under Drying Condition 2, crystalline Compound I heptane solvate Form A was dried over the weekend at 40-45° C. Under Drying Condition 3, crystalline Compound I heptane solvate Form A was dried for 4 days under house vacuum with a nitrogen bleed at 40-45° C.

The XRPD diffractograms for crystalline Compound I heptane solvate Form A samples prepared under Drying Condition 1, Drying Condition 2, and Drying Condition 3 are provided in FIG. 71, and the XRPD data are summarized below in Tables 3, 4, and 5. In FIG. 71, the top curve corresponds to Drying Condition 2, the middle curve corresponds to Drying Condition 1, and the bottom curve corresponds to Drying Condition 3. Each curve is substantially similar to each other and to the XRPD of FIG. 1.

TABLE 3
XRPD Signals for Crystalline Compound I Heptane
Solvate Form A, Drying Condition 1

| XRPD Peak No. | Angle (degrees 2-Theta ± 0.2) | Intensity % |
|---|---|---|
| 1 | 5.8572 | 100 |
| 2 | 6.0844 | 62.33 |
| 3 | 10.189 | 34.55 |
| 4 | 10.5918 | 21.42 |
| 5 | 18.5355 | 10.59 |
| 6 | 18.1599 | 10.55 |
| 7 | 12.2176 | 10.14 |

TABLE 4
XRPD Signals for Crystalline Compound I Heptane
Solvate Form A, Drying Condition 2

| XRPD Peak No. | Angle (degrees 2-Theta ± 0.2) | Intensity % |
|---|---|---|
| 1 | 5.9314 | 100 |
| 2 | 6.1405 | 52.48 |
| 3 | 10.2746 | 33.73 |
| 4 | 10.6383 | 18.32 |
| 5 | 11.9036 | 10 |

TABLE 5
XRPD Signals for Crystalline Compound I Heptane
Solvate Form A, Drying Condition 3

| XRPD Peak No. | Angle (degrees 2-Theta ± 0.2) | Intensity % |
|---|---|---|
| 1 | 6.0778 | 100 |
| 2 | 5.812 | 81.49 |
| 3 | 10.6059 | 29.34 |
| 4 | 10.1304 | 21.77 |
| 5 | 12.27 | 12.64 |

### B. Differential Scanning Calorimetry Analysis

The melting point of the product of Step 4, crystalline Compound I heptane solvate Form A, was measured using a TA Instruments Q2000 DSC.

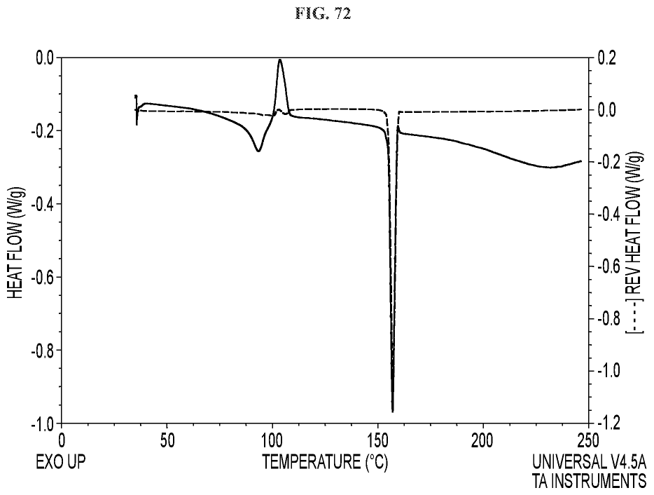

The DSC thermogram for crystalline Compound I heptane solvate Form A is provided in FIG. 72. The thermogram for crystalline Compound I heptane solvate Form A shows an endotherm at ˜93.45° C. and recrystallization at ˜103° C.

### C. Solid-State ¹³C NMR

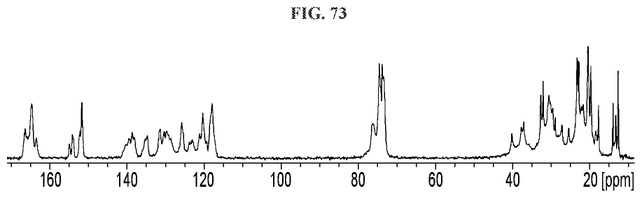

The ¹³C SSNMR of the product of Step 4, crystalline Compound I heptane solvate Form A, was acquired using the procedure described in the General SSNMR Method. The ¹³C SSNMR spectrum for crystalline Compound I heptane solvate Form A is provided in FIG. 73, and the data are summarized below in Table 6.

TABLE 6
¹³C SSNMR signals for Crystalline
Compound I Heptane Solvate Form A

| Peak # | Chem Shift [ppm] | Intensity [rel] |
|---|---|---|
| 1 | 166.3 | 26.5 |
| 2 | 165.8 | 19.3 |
| 3 | 164.6 | 48.8 |
| 4 | 163.4 | 18.2 |
| 5 | 154.8 | 12.9 |
| 6 | 154.0 | 21.1 |
| 7 | 152.1 | 24.5 |
| 8 | 151.6 | 50.5 |
| 9 | 140.2 | 12.3 |
| 10 | 139.4 | 17.5 |
| 11 | 138.5 | 23.1 |
| 12 | 138.0 | 18.9 |
| 13 | 135.1 | 17.6 |
| 14 | 134.6 | 20.4 |
| 15 | 131.3 | 26.2 |
| 16 | 130.2 | 24.2 |
| 17 | 129.6 | 24.3 |
| 18 | 128.5 | 18.0 |
| 19 | 125.7 | 31.6 |
| 20 | 123.7 | 15.1 |
| 21 | 123.2 | 14.6 |
| 22 | 122.9 | 16.7 |
| 23 | 121.1 | 21.9 |
| 24 | 120.2 | 40.6 |
| 25 | 119.2 | 15.1 |
| 26 | 117.8 | 48.6 |
| 27 | 76.2 | 31.3 |
| 28 | 74.4 | 84.8 |
| 29 | 73.7 | 84.5 |
| 30 | 73.3 | 73.1 |
| 31 | 40.0 | 22.3 |
| 32 | 38.6 | 12.0 |
| 33 | 37.6 | 27.9 |
| 34 | 36.9 | 32.7 |
| 35 | 35.7 | 12.6 |
| 36 | 33.6 | 13.5 |
| 37 | 32.5 | 57.6 |
| 38 | 32.0 | 69.7 |
| 39 | 30.4 | 56.4 |
| 40 | 30.1 | 50.3 |
| 41 | 29.5 | 44.7 |
| 42 | 28.8 | 36.63 |
| 43 | 28.1 | 21.1 |
| 44 | 27.1 | 29.6 |
| 45 | 25.3 | 27.1 |
| 46 | 23.1 | 90.9 |
| 47 | 22.7 | 88.1 |
| 48 | 22.0 | 46.4 |
| 49 | 21.6 | 48.1 |
| 50 | 20.3 | 100.0 |
| 51 | 19.6 | 83.0 |
| 52 | 18.3 | 24.6 |
| 53 | 17.6 | 47.9 |
| 54 | 13.8 | 51.6 |
| 55 | 13.1 | 39.3 |
| 56 | 12.5 | 82.3 |

### D. Solid-State ¹⁹F NMR

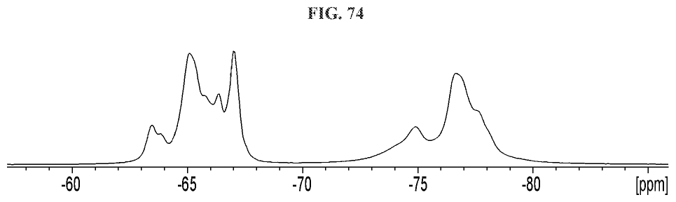

The ¹⁹F SSNMR of the product of Step 4, crystalline Compound I heptane solvate Form A, was acquired using the procedure described in the General SSNMR Method. The ¹⁹F SSNMR spectrum for crystalline Compound I heptane solvate Form A is provided in FIG. 74, and the data are summarized below in Table 7.

TABLE 7
¹⁹F SSNMR signals for Crystalline
Compound I Heptane Solvate Form A

| Peak # | Chem Shift [ppm] | Intensity [rel] |
|---|---|---|
| 1 | −63.5 | 4.4 |
| 2 | −63.8 | 3.4 |
| 3 | −65.1 | 12.2 |
| 4 | −65.8 | 7.6 |
| 5 | −66.3 | 7.8 |
| 6 | −67.0 | 12.5 |
| 7 | −74.0 | 2.1 |
| 8 | −74.9 | 4.2 |
| 9 | −76.6 | 10.0 |
| 10 | −77.6 | 5.9 |

### E. Thermogravimetric Analysis (TGA)

TGA was used to investigate the presence of residual solvents in the lots characterized and to identify the temperature at which decomposition of the sample occurs. TGA data were collected on a Mettler Toledo TGA/DSC 3+ STARe System.

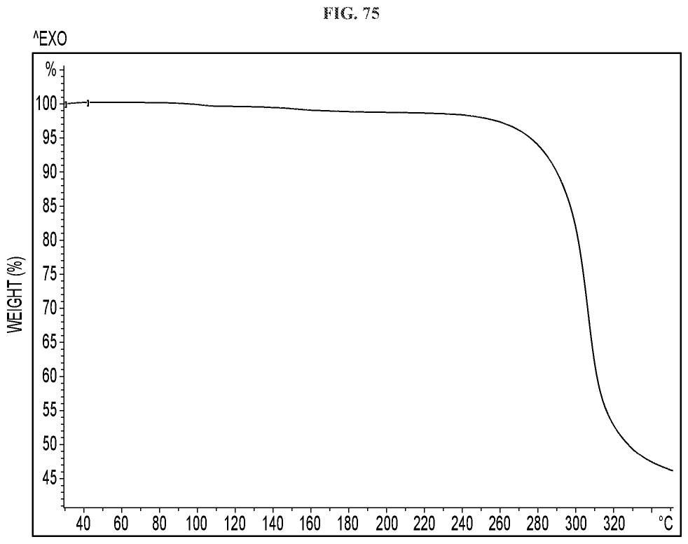

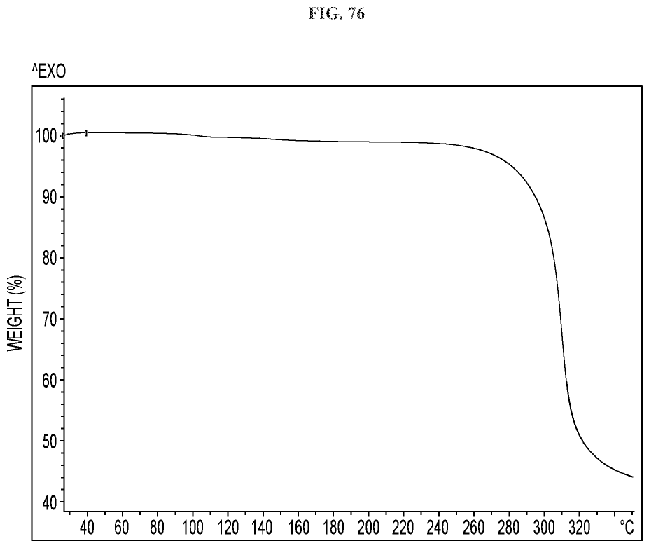

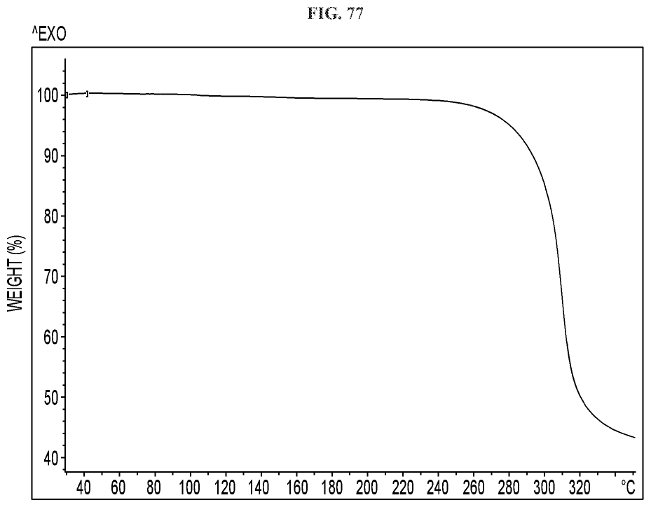

The TGA curve for crystalline Compound I heptane solvate Form A prepared under Drying Condition 1 is provided in FIG. 75. The TGA curve for crystalline Compound I heptane solvate Form A prepared under Drying Condition 2 is provided in FIG. 76. The TGA curve for crystalline Compound I heptane solvate Form A prepared under Drying Condition 3 is provided in FIG. 77. Each of the curves in FIGS. 75, 76, and 77 are substantially similar to each other.

### Example 2: Compound I Neat Amorphous Form

### A. Preparation of Compound I Neat Amorphous Form

The product of Step 3 of Example 1 (41 g, 56.27 mmol) was dissolved in ethanol (820 mL). The flask was cycled three times between vacuum/nitrogen, treated with 10% Pd/C (50% water wet, 8 g of 5% w/w, 3.759 mmol), cycled three times between vacuum/nitrogen, then cycled three times between vacuum/hydrogen. The mixture was stirred at 1200 rpm at room temperature under a hydrogen balloon for 20 h. The mixture was cycled three times between vacuum/nitrogen, filtered over celite, washed with ethanol, evaporated, and dried under high vacuum at room temperature overnight to yield an off white foam. The foam was dissolved in dichloromethane (330 mL), cooled in an ice bath and treated with trifluoroacetic acid (100 mL, 1.298 mol). The pale yellow solution was removed from the ice bath and stirred at room temperature for 2 h. The yellow solution was diluted with heptane (500 mL), evaporated (40° C.), and dried for 1 hour (40° C., 10 mbar). The yellow mass was dissolved in dichloromethane (100 ml) and diluted with heptane (500 mL) while rotating in a warm water bath (50-60° C.) to give a thick suspension. The thick yellow suspension was stirred at room temperature for 1 h, then the solids were filtered off. The mother liquor was concentrated under reduced pressure to remove most of the dichloromethane to give a yellow suspension. The solid was collected by filtration, washed with cold heptane, dried, and purified by reverse phase chromatography (415 g C18, liquid load with MeOH) with a linear gradient of 10-100% acetonitrile in water. The product fractions were evaporated and dried to yield Compound I neat amorphous form.

### B. X-Ray Powder Diffraction

The XRPD pattern for Compound I neat amorphous form was recording using the procedure described in the General XRPD Method.

The XRPD diffractogram for Compound I neat amorphous form is provided in FIG. 1.

### C. Thermogravimetric Analysis

TGA was used to investigate the presence of residual solvents in the lots characterized and identify the temperature at which decomposition of the sample occurs. TGA data for Compound I neat amorphous form was collected on a Mettler Toledo TGA/DSC 3+ STARe System.

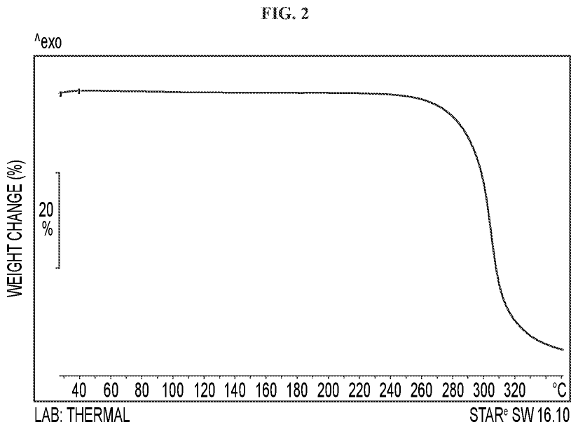

The TGA curve for Compound I neat amorphous form is provided in FIG. 2. The thermogram showed negligible weight loss from ambient temperature up until thermal degradation.

### D. Differential Scanning Calorimetry Analysis

The glass transition point of Compound I neat amorphous form was measured using a Mettler Toledo TGA/DSC 3+ STARe System.

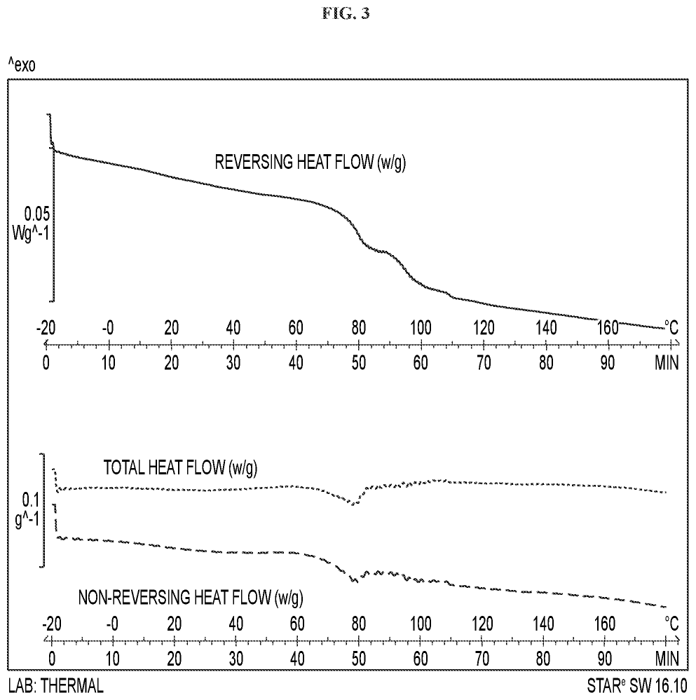

The DSC thermogram for Compound I neat amorphous form is provided in FIG. 3. Two glass transition points were observed at ˜79° C. and at ˜96° C.

### E. Solid-State ¹³C NMR

The ¹³C SSNMR of Compound I neat amorphous form was acquired using the procedure described in the General SSNMR Method. The ¹³C SSNMR spectrum for Compound I neat amorphous form is provided in FIG. 4, and the data are summarized below in Table 8.

TABLE 8
¹³C SSNMR Signals for Compound I Neat Amorphous Form

| Peak # | Chem Shift [ppm] | Intensity [rel] |
|---|---|---|
| 1 | 163.8 | 48.7 |
| 2 | 151.9 | 30.2 |
| 3 | 137.6 | 39.5 |
| 4 | 125.8 | 42.8 |
| 5 | 120.8 | 31.5 |
| 6 | 117.8 | 37.1 |
| 7 | 77.3 | 33.5 |
| 8 | 73.6 | 100.0 |
| 9 | 34.5 | 45.2 |
| 10 | 31.4 | 36.1 |
| 11 | 26.3 | 37.1 |
| 12 | 22.5 | 54.6 |
| 13 | 19.5 | 87.5 |

### F. Solid-State ¹⁹F NMR

The ¹⁹F SSNMR of Compound I neat amorphous form was acquired using the procedure described in the General SSNMR Method. The ¹⁹F SSNMR spectrum for Compound I neat amorphous form is provided in FIG. 5, and the data are summarized below in Table 9.

TABLE 9
¹⁹F SSNMR Signals for Compound I Neat Amorphous Form

| Peak # | Chem Shift [ppm] | Intensity [rel] |
|---|---|---|
| 1 | −64.6 | 12.5 |
| 2 | −77.4 | 8.7 |

### Example 3: Crystalline Compound I Neat Form A

### A. Preparation of Crystalline Compound I Neat Form A

Approximately 50 mg of crystalline Compound I neat amorphous form was weighed into a glass vial. Methanol (578 μL) was added to dissolve the solids completely. Water (422 μL) was added into the vial, and the vial was capped immediately. The system was stirred at room temperature for five days. The system was filtered and the solid was collected and dried under vacuum at 40° C. for 24 hours to yield crystalline Compound I neat Form A.

### B. X-Ray Powder Diffraction

The XRPD pattern of crystalline Compound I neat Form A was recorded using the procedure described in the General XRPD Method.

The XRPD diffractogram for crystalline Compound I neat Form A is provided in FIG. 6, and the XRPD data are summarized below in Table 10.

TABLE 10
XRPD Signals for Crystalline Compound I Neat Form A

| XRPD Peak No. | Angle (degrees 2-Theta ± 0.2) | Intensity % |
|---|---|---|
| 1 | 4.6 | 100.0 |
| 2 | 9.2 | 32.5 |
| 3 | 18.4 | 19.8 |
| 4 | 20.8 | 3.3 |

### C. Thermogravimetric Analysis

TGA was used to investigate the presence of residual solvents in the lots characterized and identify the temperature at which decomposition of the sample occurs. TGA data for crystalline Compound I neat Form A was collected on a Mettler Toledo TGA/DSC 3+ STARe System.

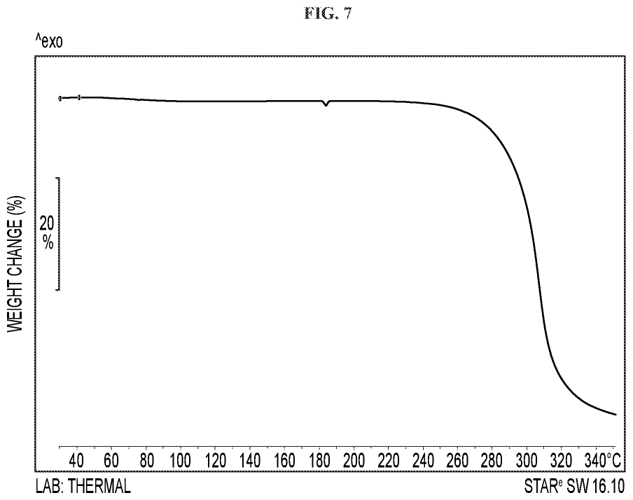

The TGA curve for crystalline Compound I neat Form A is provided in FIG. 7. The TGA thermogram showed ˜0.8% weight loss from ambient temperature up until ˜100° C.

### D. Differential Scanning Calorimetry Analysis

The melting point of crystalline Compound I neat Form A was measured using a Mettler Toledo TGA/DSC 3+ STARe System.

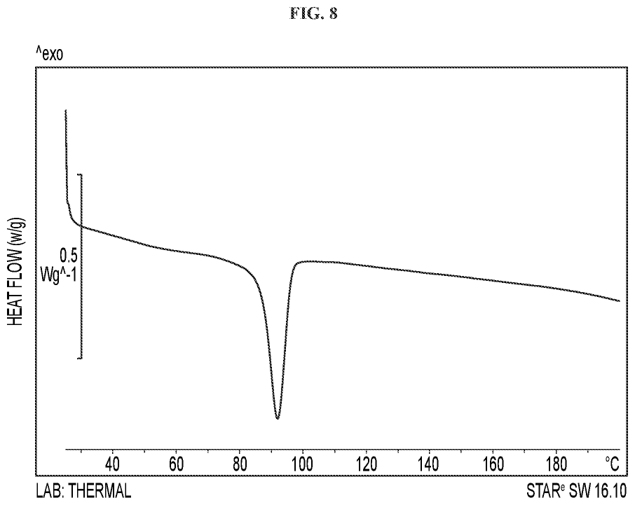

The DSC thermogram for crystalline Compound I neat Form A is provided in FIG. 8. The thermogram showed an endotherm at ˜91.7° C.

### Example 4: Crystalline Compound I Neat Form B

### A. Preparation of Crystalline Compound I Neat Form B

Approximately 60 mg of crystalline Compound I heptane solvate Form A was dissolved in dichloromethane at room temperature. The solution was evaporated slowly at room temperature to yield crystalline Compound I neat Form B.

### B. X-Ray Powder Diffraction

The XRPD pattern for crystalline Compound I neat Form B was recording using the procedure described in the General XRPD Method.

The XRPD diffractogram for crystalline Compound I neat Form B is provided in FIG. 9, and the XRPD data are summarized below in Table 11.

TABLE 11
XRPD Signals for Crystalline Compound I Neat Form B

| XRPD Peak No. | Angle (degrees 2-Theta ± 0.2) | Intensity % |
|---|---|---|
| 1 | 5.7 | 10.6 |
| 2 | 6.1 | 100.0 |
| 3 | 7.6 | 6.6 |
| 4 | 9.3 | 6.5 |
| 5 | 10.3 | 11.2 |
| 6 | 10.6 | 38.2 |
| 7 | 12.3 | 19.3 |
| 8 | 16.1 | 9.0 |

### C. Thermogravimetric Analysis

TGA was used to investigate the presence of residual solvents in the lots characterized and identify the temperature at which decomposition of the sample occurs. TGA data for crystalline Compound I neat Form B was collected on a Mettler Toledo TGA/DSC 3+ STARe System.

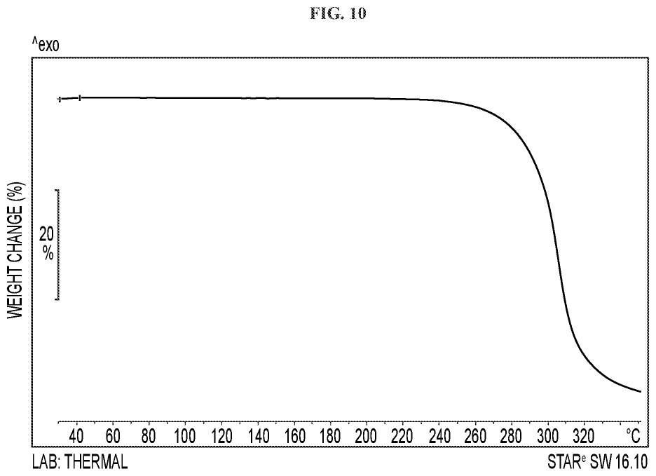

The TGA curve for crystalline Compound I neat Form B is provided in FIG. 10. The thermogram showed negligible weight loss from ambient temperature up until thermal degradation.

### D. Differential Scanning Calorimetry Analysis

The melting point of crystalline Compound I neat Form B was measured using a Mettler Toledo TGA/DSC 3+ STARe System.

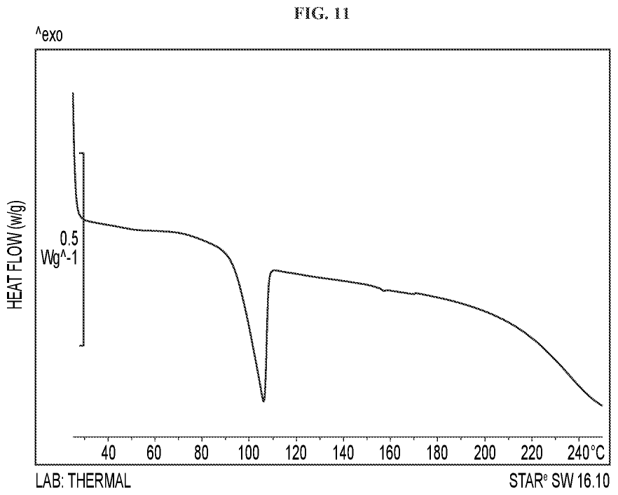

The DSC thermogram for crystalline Compound I neat Form B is provided in FIG. 11. The thermogram showed an endotherm at ˜106° C.

### E. Solid-State ¹³C NMR

The ¹³C SSNMR of crystalline Compound I neat Form B was acquired using the procedure described in the General SSNMR Method. The ¹³C SSNMR spectrum for crystalline Compound I neat Form B is provided in FIG. 12, and the data are summarized below in Table 12.

TABLE 12
¹³C SSNMR Signals for Crystalline Compound I Neat Form B

| | Chem Shift | Intensity |
|---|---|---|
| Peak # | [ppm] | [rel] |
| 1 | 165.8 | 34.4 |
| 2 | 164.7 | 50.2 |
| 3 | 163.8 | 37.7 |
| 4 | 154.2 | 26.8 |
| 5 | 151.8 | 36.4 |
| 6 | 140.1 | 18.6 |
| 7 | 138.1 | 27.1 |
| 8 | 136.2 | 18.9 |
| 9 | 134.9 | 15.3 |
| 10 | 131.7 | 20.1 |
| 11 | 129.4 | 26.7 |
| 12 | 125.5 | 40.7 |
| 13 | 123.0 | 26.7 |
| 14 | 120.2 | 40.1 |
| 15 | 117.5 | 51.3 |
| 16 | 78.3 | 20.7 |
| 17 | 74.4 | 83.5 |
| 18 | 73.6 | 100.0 |
| 19 | 37.6 | 41.2 |
| 20 | 34.0 | 29.4 |
| 21 | 29.9 | 35.3 |
| 22 | 27.3 | 34.6 |
| 23 | 22.7 | 51.0 |
| 24 | 21.1 | 61.9 |
| 25 | 18.9 | 79.3 |

### F. Solid-State ¹⁹F NMR

The ¹⁹F SSNMR of crystalline Compound I neat Form B was acquired using the procedure described in the General SSNMR Method. The ¹⁹F SSNMR spectrum for crystalline Compound I neat Form B is provided in FIG. 13, and the data are summarized below in Table 13.

TABLE 13
¹⁹F SSNMR Signals for Crystalline Compound I Neat Form B

| | Chem Shift | Intensity |
|---|---|---|
| Peak # | [ppm] | [rel] |
| 1 | − 64.3 | 8.1 |
| 2 | − 65.9 | 12.5 |
| 3 | − 76.5 | 9.1 |

### Example 5: Crystalline Compound I Hemihydrate Form C

### A. Preparation of Crystalline Compound I Hemihydrate Form C

Compound I neat Form D (3 g, 6.813 mmol) and ethanol (17.1 mL, 5.7 Vol) were added to a jacketed reactor. The solids were allowed to dissolve at 25° C. Water (68.1 mL, 0.1 M, 22.7 Vols) was added over 10-12 hours. The slurry was heated to 60° C. and the temperature was held at 60° C. for 4 hours. The slurry was cooled to 20° C. over 3 hours. The slurry was stirred for at least 2 hours. The solids were filtered and the wet cake was washed with an ethanol/water solution (675 mL, 5 volumes ethanol water 1:4 v/v). The wet cake was placed in a vacuum oven at 50° C. with a slight nitrogen bleed until a constant mass was reached, yielding 2.5 g of crystalline Compound I hemihydrate Form C (isolated yield 83.3%).

### B. X-Ray Powder Diffraction

The XRPD pattern for crystalline Compound I hemihydrate Form C was acquired at room temperature in reflection mode using a Bruker Advance equipped with Vantec-1 detector. The sample was analyzed on a silicon sample holder from 3-40° 2-theta on continuous mode with step size of 0.0144531° and time per step of 0.25 seconds. The sample was spinning at 15 rpm.

The XRPD diffractogram for crystalline Compound I hemihydrate Form C is provided in FIG. 14, and the XRPD data are summarized below in Table 14.

TABLE 14
XRPD Signals for Crystalline Compound I Hemihydrate Form C

| XRPD Peak | Angle (degrees 2- | |
|---|---|---|
| No. | Theta ± 0.2) | Intensity % |
| 1 | 21.1 | 100.0 |
| 2 | 19.6 | 59.7 |
| 3 | 16.4 | 54.4 |
| 4 | 18.4 | 47.3 |
| 5 | 13.1 | 33.7 |
| 6 | 24.6 | 31.2 |
| 7 | 4.8 | 27.8 |
| 8 | 27.1 | 26.5 |
| 9 | 19.3 | 25.1 |
| 10 | 24.0 | 21.9 |
| 11 | 11.2 | 18.1 |
| 12 | 12.5 | 17.0 |
| 13 | 9.3 | 15.2 |
| 14 | 25.8 | 13.4 |
| 15 | 33.4 | 12.5 |
| 16 | 23.5 | 11.9 |
| 17 | 22.8 | 11.5 |
| 18 | 8.2 | 11.1 |
| 19 | 29.6 | 10.9 |
| 20 | 21.6 | 10.3 |

### C. Thermogravimetric Analysis

Thermal gravimetric analysis of crystalline Compound I hemihydrate Form C was measured using a TA Instruments Q5000 TGA.

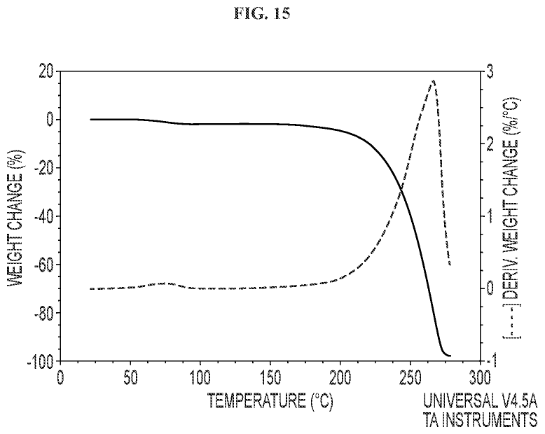

The TGA curve for crystalline Compound I hemihydrate Form C is provided in FIG. 15. The TGA thermogram shows ˜1.9% weight loss from ambient temperature up until ˜100° C.

### D. Differential Scanning Calorimetry Analysis

The melting point of crystalline Compound I hemihydrate Form C was measured using a TA Instruments Q2000 DSC.

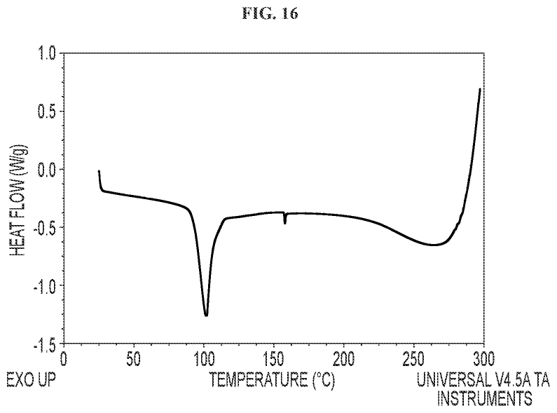

The DSC thermogram for crystalline Compound I hemihydrate Form C is provided in FIG. 16. The thermogram shows an endotherm at ˜100° C.

### E. Solid-State ¹³C NMR

The ¹³C SSNMR of crystalline Compound I hemihydrate Form C was acquired using the procedure described in the General SSNMR Method. The ¹³C SSNMR spectrum for crystalline Compound I hemihydrate Form C is provided in FIG. 17, and the data are summarized below in Table 15.

TABLE 15
¹³C SSNMR Signals for Crystalline
Compound I Hemihydrate Form C

| | Chem Shift | Intensity |
|---|---|---|
| Peak # | [ppm] | [rel] |
| 1 | 163.8 | 37.1 |
| 2 | 151.3 | 30.1 |
| 3 | 139.1 | 20.5 |
| 4 | 137.7 | 29.1 |
| 5 | 127.2 | 31.8 |
| 6 | 125.8 | 28.7 |
| 7 | 119.9 | 19.6 |
| 8 | 118.4 | 58.4 |
| 9 | 75.6 | 53.1 |
| 10 | 73.6 | 46.5 |
| 11 | 35.8 | 31.6 |
| 12 | 32.2 | 16.0 |
| 13 | 29.6 | 13.2 |
| 14 | 24.6 | 17.0 |
| 15 | 22.1 | 10.2 |
| 16 | 19.2 | 100.0 |

### F. Solid-State ¹⁹F NMR

The ¹⁹F SSNMR of crystalline Compound I hemihydrate Form C was acquired using the procedure described in the General SSNMR Method. The ¹⁹F SSNMR spectrum for crystalline Compound I hemihydrate Form C is provided in FIG. 18, and the data are summarized below in Table 16.

TABLE 16
¹⁹F SSNMR Signals for Crystalline
Compound I Hemihydrate Form C

| | Chem Shift | Intensity |
|---|---|---|
| Peak # | [ppm] | [rel] |
| 1 | − 65.5 | 12.5 |
| 2 | − 77.4 | 3.0 |

### G. Single Crystal X-Ray Diffraction

X-ray diffraction data for crystalline Compound I hemihydrate Form C were acquired at 100K on a Bruker diffractometer equipped with Cu Kα radiation (λ=1.54178 Å) and a CPAD detector. The structure was solved and refined using SHELX programs (Sheldrick, G. M., Acta Cryst., (2008) A64, 112-122) and results are summarized in Table 17 below.

TABLE 17
Single Crystal Elucidation of Crystalline
Compound I Hemihydrate Form

| Crystal System | Monoclinic |
|---|---|
| Space Group | P 2₁ |
| a (Å) | 12.1220(3) |
| b (Å) | 8.6319(3) |
| c (Å) | 18.8519(6) |
| α (°) | 90 |
| β (°) | 98.169(3) |
| γ (°) | 90 |
| V (Å3) | 1952.57(10) |
| Z/Z′ | 2/1 |
| Temperature (K) | 100(2) |
| Density (g/cm3) | 1.515 |

### Example 6: Crystalline Compound I Neat Form D

### A. Preparation of Crystalline Compound I Neat Form D

Approximately 25 mg of crystalline Compound I hemihydrate Form C was added to a 2 mL HPLC vial, followed by 30 μL of ethanol. The solution was mixed by vortexer until the solids were dissolved. The solution was transferred to the XRPD plate. The XRPD plate was placed under nitrogen for a half hour, then placed in an oven at 80° C. for ˜5 days. Large crystals of crystalline Compound I neat Form D appeared.

In an alternative procedure, a 400 mL reactor was charged with crystalline Compound I hemihydrate Form C (2.8 g, 6.359 mmol, 1 equiv.) and n-heptane (49 mL, 0.13 M, 17.5 Vols). The slurry was heated to 85° C. The slurry was charged with a seed of crystalline Compound I neat Form D (0.053 g). The slurry was held at 85±5° C. until full conversion was verified by XRPD. The slurry was cooled to 65° C. over 4 hours. The solids were filtered and the wet cake was washed with n-heptane (12.5 mL, 5 vol). The wet cake was placed in a vacuum oven at 50° C. with a slight nitrogen bleed until a constant mass was reached, yielding 2.4 g of crystalline Compound I neat Form D (isolated yield 85.7%).

### B. X-Ray Powder Diffraction

The XRPD pattern for crystalline Compound I neat Form D was acquired at room temperature in reflection mode using a Bruker Advance equipped with Vantec-1 detector. The sample was analyzed on a silicon sample holder from 3-40° 2-theta on continuous mode with step size of 0.0144531° and time per step of 0.25 seconds. The sample was spinning at 15 rpm.

The XRPD diffractogram for crystalline Compound I neat Form D is provided in FIG. 19, and the XRPD data are summarized below in Table 18.

TABLE 18
XRPD Signals for Crystalline Compound I Neat Form D

| XRPD Peak | Angle (degrees 2- | |
|---|---|---|
| No. | Theta ± 0.2) | Intensity % |
| 1 | 20.3 | 100.0 |
| 2 | 26.45 | 93.9 |
| 3 | 16.85 | 72.5 |
| 4 | 16.77 | 60.5 |
| 5 | 26.52 | 53.3 |
| 6 | 8.4 | 44.7 |
| 7 | 25.2 | 37.2 |
| 8 | 20.0 | 29.3 |
| 9 | 16.0 | 27.5 |
| 10 | 14.3 | 23.0 |
| 11 | 15.4 | 20.4 |
| 12 | 21.7 | 20.0 |
| 13 | 28.8 | 18.7 |
| 14 | 18.64 | 16.0 |
| 15 | 19.6 | 15.9 |
| 16 | 8.8 | 15.3 |
| 17 | 26.2 | 13.8 |
| 18 | 27.8 | 13.0 |
| 19 | 22.5 | 12.7 |
| 20 | 18.55 | 11.3 |
| 21 | 24.7 | 10.9 |
| 22 | 14.8 | 10.9 |

### C. Thermogravimetric Analysis

Thermal gravimetric analysis of crystalline Compound I neat Form D was measured using a TA5500 Discovery TGA.

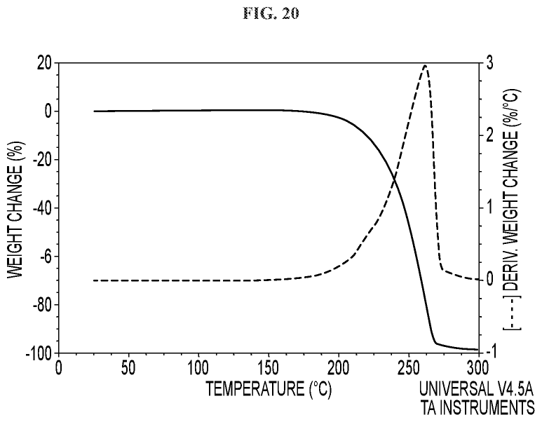

The TGA curve for crystalline Compound I neat Form D is provided in FIG. 20. The TGA thermogram shows negligible weight loss from ambient temperature up until thermal degradation.

### D. Differential Scanning Calorimetry Analysis (Room Temperature)

The melting point of crystalline Compound I neat Form D at room temperature was measured using a TA Instruments Q2000 DSC.

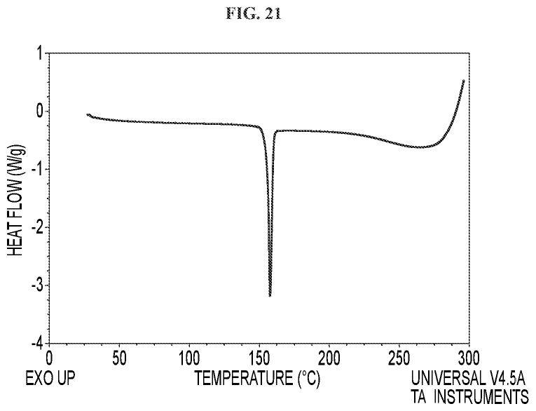

The DSC thermogram for crystalline Compound I neat Form D at room temperature is provided in FIG. 21. The thermogram shows an endotherm at ˜158° C.

### E. Solid-State ¹³C NMR

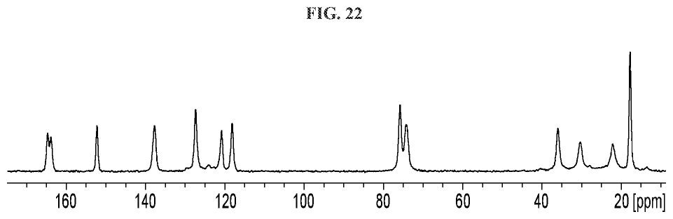

The ¹³C SSNMR of crystalline Compound I neat Form D was acquired using the procedure described in the General SSNMR Method. The ¹³C SSNMR spectrum for crystalline Compound I neat Form D is provided in FIG. 22, and the data are summarized below in Table 19.

TABLE 19
¹³C SSNMR Signals for Crystalline Compound I Neat Form D

| | Chem Shift | Intensity |
|---|---|---|
| Peak # | [ppm] | [rel] |
| 1 | 164.6 | 31.7 |
| 2 | 163.8 | 28.6 |
| 3 | 152.2 | 38.1 |
| 4 | 137.7 | 38.1 |
| 5 | 127.3 | 52.1 |
| 6 | 120.8 | 33.8 |
| 7 | 118.1 | 40.3 |
| 8 | 75.7 | 56.2 |
| 9 | 74.2 | 39.4 |
| 10 | 35.9 | 35.8 |
| 11 | 30.4 | 24.3 |
| 12 | 22.1 | 22.7 |
| 13 | 17.7 | 100.0 |

### F. Solid-State ¹⁹F NMR

The ¹⁹F SSNMR of crystalline Compound I neat Form D was acquired using the procedure described in the General SSNMR Method. The ¹⁹F SSNMR spectrum for crystalline Compound I neat Form D is provided in FIG. 23, and the data are summarized below in Table 20.

TABLE 20
19F SSNMR Signals for Crystalline Compound I Neat Form D

| | Chem Shift | Intensity |
|---|---|---|
| Peak # | [ppm] | [rel] |
| 1 | − 62.4 | 12.5 |
| 2 | − 77.2 | 1.2 |

### G. Single Crystal X-Ray Diffraction

X-ray diffraction data for crystalline Compound I neat Form D were acquired at 250 K on a Bruker diffractometer equipped with Cu Kα radiation (λ=1.54178 Å) and a CPAD detector. The structure was solved and refined using SHELX programs (Sheldrick, G. M., Acta Cryst., (2008) A64, 112-122) and results are summarized in Table 21 below.

TABLE 21
Single Crystal Elucidation of Crystalline Compound I Neat Form D

| Crystal System | Monoclinic |
|---|---|
| Space Group | P 2₁ |
| a (Å) | 7.9371(1) |
| b (Å) | 11.4870(2) |
| c (Å) | 20.9850(3) |
| α (°) | 90 |
| β (0) | 90.015(2) |
| γ (*) | 90 |
| V (Å3) | 1913.28(5) |
| Z/Z′ | 2/2 |
| Temperature (K) | 250(2) |

### Example 7: Crystalline Compound I Neat Form E

### A. Preparation of Crystalline Compound I Neat Form E

Crystalline Compound I neat Form D was cooled to 100 K to yield crystalline Compound I neat Form E.

### B. Differential Scanning Calorimetry Analysis

The melting point of crystalline Compound I neat Form E was measured using a Discovery TA Instruments DSC 2500.

The DSC thermogram for crystalline Compound I neat Form E is provided in FIG. 24. The thermogram shows an endotherm at approximately −44° C.

### C. Single Crystal X-Ray Diffraction

X-ray diffraction data for crystalline Compound I neat Form E were acquired by cooling crystalline Compound I neat Form D to 100 K on a Bruker diffractometer equipped with Cu Kα radiation (λ=1.54178 Å) and a CPAD detector. The structure was solved and refined using SHELX programs (Sheldrick, G. M., Acta Cryst., (2008) A64, 112-122) and results are summarized in Table 22 below.

TABLE 22
Single Crystal Elucidation of Crystalline Compound I Neat Form E

| Crystal System | Orthorhombic |
|---|---|
| Space Group | P 2₁2₁2₁ |
| a (Å) | 8.2636(4) |
| b (A) | 11.1838(5) |
| c (Å) | 20.2227(10) |
| α (°) | 90 |
| β (°) | 90 |
| γ (°) | 90 |
| V (Å3) | 1868.95(15) |
| Z/Z′ | 4/1 |
| Temperature (K) | 100(2) |

### Example 8: Crystalline Compound I Acetic Acid Solvate

### A. Preparation of Crystalline Compound I Acetic Acid Solvate

Approximately 15 mg of crystalline Compound I hemihydrate Form C was placed into a Precellys ball milling tube, and 2 μl of acetic acid was added. The ball milling was carried out with these settings: 7500 rpm, 10 second, 2 cycles, pause 60 s after each cycle to yield crystalline Compound I acetic acid solvate.

### B. X-Ray Powder Diffraction

The XRPD pattern for crystalline Compound I acetic acid solvate was acquired at room temperature in transmission mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 2θ with a step size of 0.0131303° and 49 s per step.

The XRPD diffractogram for crystalline Compound I acetic acid solvate is provided in FIG. 25, and the XRPD data are summarized below in Table 23.

TABLE 23
XRPD Signals for Crystalline Compound I Acetic Acid Solvate

| XRPD Peak | Angle (degrees 2- | |
|---|---|---|
| No. | Theta ± 0.2) | Intensity % |
| 1 | 20.2 | 100.0 |
| 2 | 14.2 | 57.6 |
| 3 | 5.4 | 50.4 |
| 4 | 15.9 | 43.5 |
| 5 | 10.2 | 26.7 |
| 6 | 19.8 | 26.7 |
| 7 | 18.5 | 26.5 |
| 8 | 20.6 | 22.0 |
| 9 | 21.6 | 20.2 |
| 10 | 19.5 | 19.6 |
| 11 | 13.2 | 18.8 |
| 12 | 17.5 | 18.8 |
| 13 | 10.9 | 18.1 |
| 14 | 26.3 | 15.4 |
| 15 | 25.0 | 15.3 |
| 16 | 11.7 | 14.6 |
| 17 | 16.7 | 14.2 |
| 18 | 18.0 | 14.0 |
| 19 | 13.8 | 13.9 |
| 20 | 8.3 | 12.9 |
| 21 | 22.5 | 11.9 |
| 22 | 13.1 | 10.7 |
| 23 | 25.3 | 10.6 |
| 24 | 10.4 | 10.3 |
| 25 | 18.9 | 10.1 |

### C. Differential Scanning Calorimetry Analysis

The melting point of crystalline Compound I acetic acid solvate was measured using a TA Instruments Q2000 DSC.

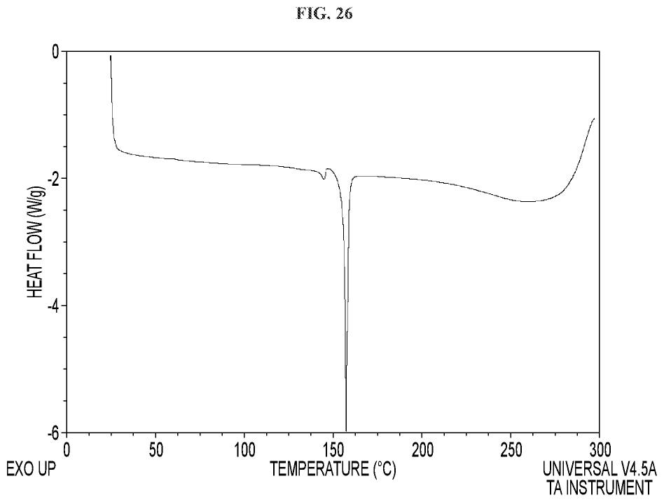

The DSC thermogram for crystalline Compound I acetic acid solvate is provided in FIG. 26. The thermogram shows an endotherm at ˜145° C. and ˜158° C.

### Example 9: Crystalline Compound I Heptane Solvate Form B

### A. Preparation of Crystalline Compound I Heptane Solvate Form B

An HPLC vial with a crimped top was charged with crystalline Compound I neat Form D (82.5 mg) and 1-butanol/heptane (0.5 mL, 75 v %0 heptane). The sealed vial was placed in a shaker block at 25° C. The vial was stirred for 2 days to yield crystalline Compound I heptane solvate Form B.

### B. X-Ray Powder Diffraction

The XRPD pattern for crystalline Compound I heptane solvate Form B was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I heptane solvate Form B is provided in FIG. 27, and the XRPD data are summarized below in Table 24.

TABLE 24
XRPD Signals for Crystalline Compound
I Heptane Solvate Form B

| XRPD Peak | Angle (degrees 2- | |
|---|---|---|
| No. | Theta ± 0.2) | Intensity % |
| 1 | 20.4 | 100.0 |
| 2 | 10.9 | 58.9 |
| 3 | 10.1 | 47.3 |
| 4 | 8.9 | 37.3 |
| 5 | 18.8 | 35.4 |
| 6 | 7.3 | 33.6 |
| 7 | 8.1 | 30.2 |
| 8 | 4.4 | 22.4 |
| 9 | 17.9 | 19.0 |
| 10 | 21.9 | 18.1 |
| 11 | 24.5 | 15.3 |
| 12 | 14.7 | 14.1 |
| 13 | 23.2 | 13.8 |
| 14 | 11.1 | 11.7 |
| 15 | 23.8 | 11.1 |
| 16 | 25.6 | 10.7 |
| 17 | 14.4 | 10.5 |

### C. Differential Scanning Calorimetry Analysis

The melting point of crystalline Compound I heptane solvate Form B was measured using a TA Instruments Q2000 DSC.

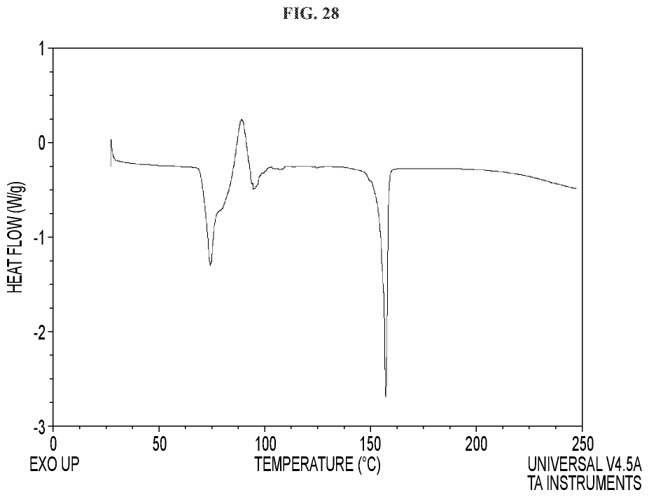

The DSC thermogram for crystalline Compound I heptane solvate Form B is provided in FIG. 28. The thermogram shows endotherms at ˜75, ˜94 and ˜157° C.

### D. Solid-State ¹³C NMR

The ¹³C SSNMR of crystalline Compound I heptane solvate Form B was acquired using the procedure described in the General SSNMR Method. The ¹³C SSNMR spectrum for crystalline Compound I heptane solvate Form B is provided in FIG. 29, and the data are summarized below in Table 25.

TABLE 25
¹³C SSNMR Signals for Crystalline
Compound I Heptane Solvate Form B

| | Chem Shift | Intensity |
|---|---|---|
| Peak # | [ppm] | [rel] |
| 1 | 164.4 | 42.5 |
| 2 | 163.0 | 66.7 |
| 3 | 151.0 | 69.0 |
| 4 | 139.5 | 40.9 |
| 5 | 137.5 | 40.0 |
| 6 | 126.3 | 66.0 |
| 7 | 120.8 | 44.7 |
| 8 | 120.0 | 30.3 |
| 9 | 117.4 | 78.5 |
| 10 | 75.5 | 38.3 |
| 11 | 74.7 | 50.1 |
| 12 | 74.1 | 47.1 |
| 13 | 73.0 | 38.4 |
| 14 | 34.2 | 18.7 |
| 15 | 31.1 | 86.8 |
| 16 | 28.2 | 39.6 |
| 17 | 22.4 | 77.5 |
| 18 | 20.8 | 72.8 |
| 19 | 19.5 | 100.0 |
| 20 | 13.8 | 97.8 |

### E. Solid-State ¹⁹F NMR

The ¹⁹F SSNMR of crystalline Compound I heptane solvate Form B was acquired using the procedure described in the General SSNMR Method. The ¹⁹F SSNMR spectrum for crystalline Compound I heptane solvate Form B is provided in FIG. 30, and the data are summarized below in Table 26.

TABLE 26
¹⁹F SSNMR Signals for Crystalline
Compound I Heptane Solvate Form B

| | Chem Shift | Intensity |
|---|---|---|
| Peak # | [ppm] | [rel] |
| 1 | − 63.4 | 9.9 |
| 2 | − 64.2 | 12.5 |
| 3 | − 77.4 | 7.4 |
| 4 | − 78.4 | 5.8 |

### Example 10: Crystalline Compound I Heptane Solvate Form C

### A. Preparation of Crystalline Compound I Heptane Solvate Form C

An HPLC vial with a crimped top was charged with crystalline Compound I neat Form D (59.6 mg) and ethyl acetate/heptane (0.5 mL, 25 v % heptane). The sealed vial was placed in a shaker block at 25° C. The vial was stirred for 2 days to yield crystalline Compound I heptane solvate Form C.

### B. X-Ray Powder Diffraction

The XRPD pattern for crystalline Compound I heptane solvate Form C was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.01313030 and 49 s per step.

The XRPD diffractogram for crystalline Compound I heptane solvate Form C is provided in FIG. 31, and the XRPD data are summarized below in Table 27.

TABLE 27
XRPD Signals for Crystalline Compound
I Heptane Solvate Form C

| | Angle (degrees 2- | |
|---|---|---|
| XRPD Peak No. | Theta ± 0.2) | Intensity % |
| 1 | 20.4 | 100.0 |
| 2 | 13.1 | 28.6 |
| 3 | 11.6 | 22.5 |
| 4 | 8.2 | 14.4 |
| 5 | 32.3 | 13.5 |
| 6 | 5.5 | 13.5 |
| 7 | 8.0 | 12.8 |
| 8 | 9.3 | 11.2 |

### C. Thermogravimetric Analysis

Thermal gravimetric analysis of crystalline Compound I heptane solvate Form C was measured using a TA5500 Discovery TGA.

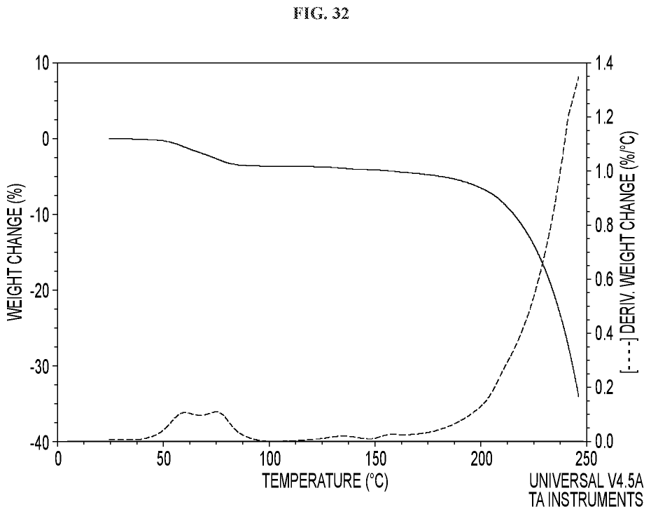

The TGA curve for crystalline Compound I heptane solvate Form C is provided in FIG. 32. The TGA thermogram shows ˜3.6% weight loss from ambient temperature to 100° C.

### D. Differential Scanning Calorimetry Analysis

The melting point of crystalline Compound I heptane solvate Form C was measured using a TA Instruments Q2000 DSC.

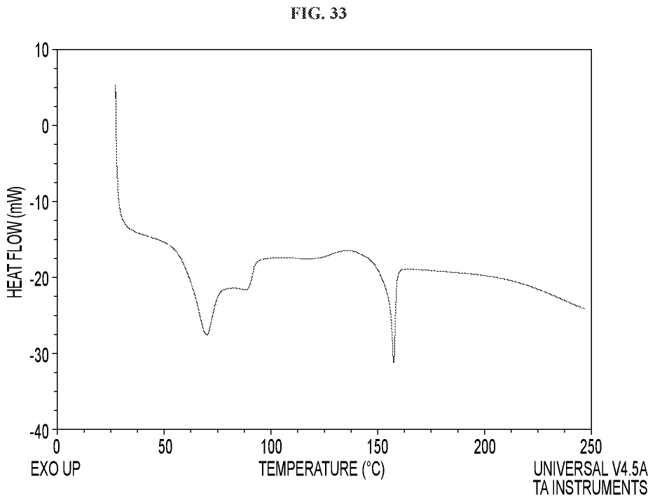

The DSC thermogram for crystalline Compound I heptane solvate Form C is provided in FIG. 33. The thermogram shows endotherms at ˜69, ˜90, ˜124 and ˜158° C.

### E. Solid-State ¹³C NMR

The ¹³C SSNMR of crystalline Compound I heptane solvate Form C was acquired using the procedure described in the General SSNMR Method. The ¹³C SSNMR spectrum for crystalline Compound I heptane solvate Form C is provided in FIG. 34, and the data are summarized below in Table 28.

TABLE 28
¹³C SSNMR Signals for Crystalline
Compound I Heptane Solvate Form C

| | Chem Shift | |
|---|---|---|
| Peak # | [ppm] | Intensity [rel] |
| 1 | 164.4 | 43.9 |
| 2 | 163.6 | 41.0 |
| 3 | 163.1 | 39.1 |
| 4 | 151.1 | 51.1 |
| 5 | 139.4 | 51.2 |
| 6 | 128.3 | 24.8 |
| 7 | 126.9 | 53.9 |
| 8 | 124.1 | 15.6 |
| 9 | 121.5 | 14.1 |
| 10 | 118.8 | 68.7 |
| 11 | 117.9 | 43.5 |
| 12 | 76.1 | 42.6 |
| 13 | 73.6 | 99.7 |
| 14 | 71.5 | 31.5 |
| 15 | 37.0 | 32.8 |
| 16 | 36.1 | 48.0 |
| 17 | 33.6 | 15.2 |
| 18 | 30.9 | 41.9 |
| 19 | 27.9 | 26.3 |
| 20 | 24.3 | 45.2 |
| 21 | 23.3 | 39.0 |
| 22 | 21.0 | 21.4 |
| 23 | 20.0 | 65.7 |
| 24 | 18.1 | 100.0 |
| 25 | 14.2 | 19.4 |

### Example 11: Crystalline Compound I Octane Solvate

### A. Preparation of Crystalline Compound I Octane Solvate

Approximately 20 mg of crystalline Compound I neat Form C was weighed into an HPLC vial. Octane (500 μL) was added. The mixture was stirred with a magnetic stirrer and placed in shaker block at 35° C. for one week to yield crystalline Compound I octane solvate.

### B. X-Ray Powder Diffraction

The XRPD diffractogram of crystalline Compound I octane solvate was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I octane solvate is provided in FIG. 35, and the XRPD data are summarized below in Table 29.

TABLE 29
XRPD Signals for Crystalline Compound I Octane Solvate

| | Angle (degrees 2- | |
|---|---|---|
| XRPD Peak No. | Theta ± 0.2) | Intensity % |
| 1 | 5.9 | 100.0 |
| 2 | 10.2 | 28.7 |
| 3 | 18.2 | 15.5 |
| 4 | 5.6 | 13.9 |
| 5 | 11.7 | 12.7 |
| 6 | 20.5 | 11.5 |
| 7 | 18.5 | 10.2 |

### C. Solid-State ¹³C NMR

The ¹³C SSNMR of crystalline Compound I octane solvate was acquired using the procedure described in the General SSNMR Method. The ¹³C SSNMR spectrum for crystalline Compound I octane solvate is provided in FIG. 36, and the data are summarized below in Table 30.

TABLE 30
¹³C SSNMR Signals for Crystalline Compound I Octane Solvate

| | Chem Shift | |
|---|---|---|
| Peak # | [ppm] | Intensity [rel] |
| 1 | 166.3 | 14.9 |
| 2 | 164.6 | 61.5 |
| 3 | 164.1 | 37.3 |
| 4 | 153.8 | 9.5 |
| 5 | 152.2 | 53.5 |
| 6 | 151.7 | 25.0 |
| 7 | 140.4 | 9.6 |
| 8 | 137.6 | 43.1 |
| 9 | 135.3 | 13.6 |
| 10 | 134.8 | 12.4 |
| 11 | 131.1 | 19.1 |
| 12 | 130.2 | 19.3 |
| 13 | 127.3 | 51.1 |
| 14 | 125.5 | 22.7 |
| 15 | 122.7 | 14.6 |
| 16 | 120.8 | 48.6 |
| 17 | 120.1 | 26.1 |
| 18 | 118.1 | 76.5 |
| 19 | 75.7 | 58.4 |
| 20 | 74.4 | 77.8 |
| 21 | 73.8 | 77.3 |
| 22 | 40.2 | 15.6 |
| 23 | 37.5 | 18.2 |
| 24 | 36.1 | 31.7 |
| 25 | 32.0 | 47.2 |
| 26 | 29.9 | 71.1 |
| 27 | 28.5 | 37.3 |
| 28 | 27.0 | 32.6 |
| 29 | 25.1 | 19.4 |
| 30 | 22.4 | 78.3 |
| 31 | 20.0 | 52.1 |
| 32 | 17.7 | 100.0 |
| 33 | 14.1 | 27.4 |
| 34 | 13.5 | 26.6 |
| 35 | 12.6 | 17.8 |

### D. Solid-State ¹⁹F NMR

The ¹⁹F SSNMR of crystalline Compound I octane solvate was acquired using the procedure described in the General SSNMR Method. The ¹⁹F SSNMR spectrum for crystalline Compound I octane solvate is provided in FIG. 37, and the data are summarized below in Table 31.

TABLE 31
¹⁹F SSNMR Signals for Crystalline Compound I Octane Solvate

| | Chem Shift | |
|---|---|---|
| Peak # | [ppm] | Intensity [rel] |
| 1 | −62.5 | 12.5 |
| 2 | −65.0 | 2.6 |
| 3 | −65.6 | 2.6 |
| 4 | −66.2 | 1.6 |
| 5 | −67.1 | 2.3 |
| 6 | −75.1 | 1.7 |
| 7 | −76.5 | 2.9 |
| 8 | −77.2 | 3.4 |

### Example 12: Crystalline Compound I Cyclohexane Solvate Form A

### A. Preparation of Crystalline Compound I Cyclohexane Solvate Form A

An HPLC vial with a crimped top was charged with crystalline Compound I neat Form D (57 mg) and cyclohexane (0.5 mL). The sealed vial was placed in a shaker block at 25° C. The vial was stirred for 3 days to yield crystalline Compound I cyclohexane solvate Form A.

### B. X-Ray Powder Diffraction

The XRPD diffractogram of crystalline Compound I cyclohexane solvate Form A was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I cyclohexane solvate Form A is provided in FIG. 38, and the XRPD data are summarized below in Table 32.

TABLE 32
XRPD Signals for Crystalline Compound
I Cyclohexane Solvate Form A

| | Angle (degrees 2- | |
|---|---|---|
| XRPD Peak No. | Theta ± 0.2) | Intensity % |
| 1 | 21.6 | 100.0 |
| 2 | 33.6 | 70.4 |
| 3 | 5.1 | 68.6 |
| 4 | 5.6 | 54.1 |
| 5 | 16.0 | 22.3 |
| 6 | 19.9 | 13.5 |
| 7 | 18.5 | 11.2 |
| 8 | 16.7 | 10.9 |

### C. Solid-State ¹³C NMR

The ¹³C SSNMR of crystalline Compound I cyclohexane solvate Form A was acquired using the procedure described in the General SSNMR Method. The ¹³C SSNMR spectrum for crystalline Compound I cyclohexane solvate Form A is provided in FIG. 39, and the data are summarized below in Table 33.

TABLE 33
¹³C SSNMR Signals for Crystalline Compound
I Cyclohexane Solvate Form A

| | Chem Shift | |
|---|---|---|
| Peak # | [ppm] | Intensity [rel] |
| 1 | 166.6 | 0.7 |
| 2 | 164.7 | 11.5 |
| 3 | 163.7 | 8.6 |
| 4 | 154.4 | 2.9 |
| 5 | 152.1 | 11.1 |
| 6 | 150.8 | 2.7 |
| 7 | 140.4 | 3.0 |
| 8 | 138.8 | 4.5 |
| 9 | 137.6 | 8.0 |
| 10 | 135.4 | 2.0 |
| 11 | 131.5 | 4.4 |
| 12 | 127.3 | 8.0 |
| 13 | 125.5 | 3.6 |
| 14 | 123.4 | 1.6 |
| 15 | 120.7 | 8.1 |
| 16 | 119.7 | 5.8 |
| 17 | 118.1 | 14.6 |
| 18 | 75.7 | 12.6 |
| 19 | 74.3 | 10.5 |
| 20 | 73.4 | 13.6 |
| 21 | 37.4 | 4.6 |
| 22 | 36.2 | 3.7 |
| 23 | 30.6 | 6.0 |
| 24 | 27.4 | 100.0 |
| 25 | 21.9 | 7.7 |
| 26 | 19.4 | 13.6 |
| 27 | 17.7 | 14.8 |

### D. Solid-State ¹⁹F NMR

The ¹⁹F SSNMR of crystalline Compound I cyclohexane solvate Form A was acquired using the procedure described in the General SSNMR Method. The ¹⁹F SSNMR spectrum for crystalline Compound I cyclohexane solvate Form A is provided in FIG. 40, and the data are summarized below in Table 34.

TABLE 34
¹⁹F SSNMR Signals for Crystalline Compound
I Cyclohexane Solvate Form A

| | Chem Shift | |
|---|---|---|
| Peak # | [ppm] | Intensity [rel] |
| 1 | −62.6 | 12.5 |
| 2 | −64.5 | 6.9 |
| 3 | −65.9 | 3.2 |
| 4 | −66.8 | 3.8 |
| 5 | −75.4 | 1.9 |
| 6 | −76.6 | 4.2 |
| 7 | −77.6 | 2.8 |

### Example 13: Crystalline Compound I Cyclohexane Solvate Form B

### A. Preparation of Crystalline Compound I Cyclohexane Solvate Form B

An HPLC vial with a crimped top was charged with crystalline Compound I hemihydrate Form C (49 mg) and cyclohexane (0.5 mL). The sealed vial was placed in a shaker block at 80° C. The vial was stirred for 3 days. The solids were filtered and collected to yield crystalline Compound I cyclohexane solvate Form B.

### B. X-Ray Powder Diffraction

The powder, X-ray powder diffraction (XRPD), diffractogram of crystalline Compound I cyclohexane solvate Form B was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I cyclohexane solvate Form B is provided in FIG. 41 and the XRPD data are summarized below in Table 35.

TABLE 35
XRPD Signals for Crystalline Compound
I Cyclohexane Solvate Form B

| | Angle (degrees 2- | |
|---|---|---|
| XRPD Peak No. | Theta ± 0.2) | Intensity % |
| 1 | 7.8 | 100.0 |
| 2 | 19.9 | 60.9 |
| 3 | 11.8 | 37.6 |
| 4 | 13.8 | 27.9 |
| 5 | 18.4 | 19.7 |
| 6 | 20.8 | 19.1 |
| 7 | 23.4 | 13.8 |
| 8 | 16.6 | 12.9 |
| 9 | 21.6 | 12.2 |
| 10 | 15.5 | 11.9 |
| 11 | 26.7 | 11.0 |

### C. Differential Scanning Calorimetry Analysis

The melting point of crystalline Compound I cyclohexane solvate Form B was measured using a TA Instruments Q2000 DSC.

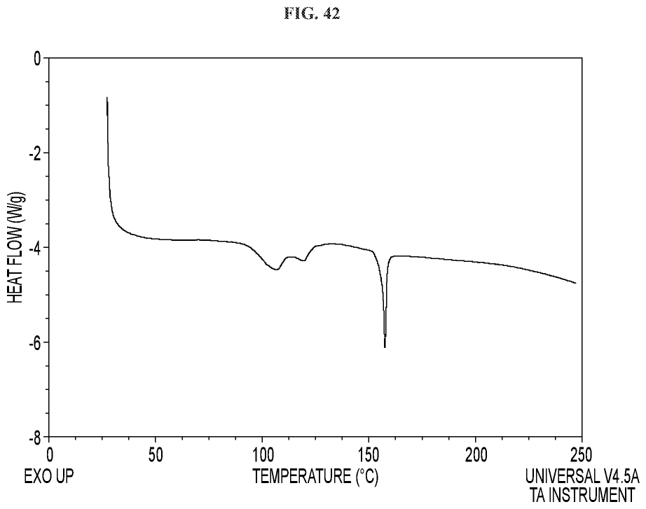

The DSC thermogram for crystalline Compound I cyclohexane solvate Form B is provided in FIG. 42. The thermogram shows an endotherms at ˜105, ˜120 and ˜158° C.

### D. Solid-State ¹³C NMR

The ¹³C SSNMR of crystalline Compound I cyclohexane solvate Form B was acquired using the procedure described in the General SSNMR Method. The ¹³C SSNMR spectrum for crystalline Compound I cyclohexane solvate Form B is provided in FIG. 43, and the data are summarized below in Table 36.

TABLE 36
¹³C SSNMR Signals for Crystalline Compound
I Cyclohexane Solvate Form B

| | Chem Shift | |
|---|---|---|
| Peak # | [ppm] | Intensity [rel] |
| 1 | 164.7 | 21.5 |
| 2 | 150.9 | 21.5 |
| 3 | 138.7 | 29.8 |
| 4 | 128.0 | 30.7 |
| 5 | 118.2 | 40.3 |
| 6 | 75.6 | 37.1 |
| 7 | 73.6 | 26.5 |
| 8 | 36.5 | 19.4 |
| 9 | 34.7 | 19.0 |
| 10 | 31.5 | 22.3 |
| 11 | 26.5 | 100.0 |
| 12 | 19.5 | 55.7 |
| 13 | 19.0 | 57.6 |

### E. Solid-State ¹⁹F NMR

The ¹⁹F SSNMR of crystalline Compound I cyclohexane solvate Form B was acquired using the procedure described in the General SSNMR Method. The ¹⁹F SSNMR spectrum for crystalline Compound I cyclohexane solvate Form B is provided in FIG. 44, and the data are summarized below in Table 37.

TABLE 37
¹⁹F SSNMR Signals for Crystalline Compound
I Cyclohexane Solvate Form B

| | Chem Shift | |
|---|---|---|
| Peak # | [ppm] | Intensity [rel] |
| 1 | −64.3 | 12.5 |
| 2 | −75.0 | 1.0 |

### Example 14: Crystalline Compound I Cyclohexane Solvate Form C

### A. Preparation of Crystalline Compound I Cyclohexane Solvate Form C

Approximately 20 mg of crystalline Compound I hemihydrate Form C was weighed into an HPLC vial. Cyclohexane (500 μL) was added. The mixture was stirred with a magnetic stirrer and placed in shaker block at 50° C. for one week to yield crystalline Compound I cyclohexane solvate Form C.

### B. X-Ray Powder Diffraction

The XRPD diffractogram of crystalline Compound I cyclohexane solvate Form C was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I cyclohexane solvate Form C is provided in FIG. 45, and the XRPD data are summarized below in Table 38.

TABLE 38
XRPD Signals for Crystalline Compound
I Cyclohexane Solvate Form C

| | Angle (degrees 2- | |
|---|---|---|
| XRPD Peak No. | Theta ± 0.2) | Intensity % |
| 1 | 5.8 | 100.0 |
| 2 | 7.8 | 36.4 |
| 3 | 19.9 | 23.6 |
| 4 | 10.0 | 22.4 |
| 5 | 11.8 | 14.1 |
| 6 | 13.9 | 12.6 |

### Example 15: Crystalline Compound I Ethanol Solvate

### A. Preparation of Crystalline Compound I Ethanol Solvate

Approximately 50 mg of crystalline Compound I hemihydrate Form C was weighed in a HPLC vial and ˜30 μL of ethanol was added. The mixture was stirred with a magnetic stirrer and kept at −20° C. to yield crystalline Compound I ethanol solvate.

### B. X-Ray Powder Diffraction

The XRPD pattern for crystalline crystalline Compound I ethanol solvate was acquired at room temperature in reflection mode using a Bruker Advance equipped with Vantec-1 detector. The sample was analyzed on a silicon sample holder from 3-40° 2-theta on continuous mode with step size of 0.0144531° and time per step of 0.25 seconds. The sample was spinning at 15 rpm.

The XRPD diffractogram for crystalline Compound I ethanol solvate is provided in FIG. 46, and the XRPD data are summarized below in Table 39.

TABLE 39
XRPD Signals for Crystalline Compound I Ethanol Solvate

| | Angle (degrees 2- | |
|---|---|---|
| XRPD Peak No. | Theta ± 0.2) | Intensity % |
| 1 | 6.2 | 100.0 |
| 2 | 7.8 | 15.8 |
| 3 | 13.3 | 10.1 |

### C. Solid-State ¹³C NMR

The ¹³C SSNMR of crystalline Compound I ethanol solvate was acquired using the procedure described in the General SSNMR Method. The ¹³C SSNMR spectrum for crystalline Compound I ethanol solvate is provided in FIG. 47, and the data are summarized below in Table 40.

TABLE 40
¹³C SSNMR Signals for Crystalline Compound I Ethanol Solvate

| | Chem Shift | |
|---|---|---|
| Peak # | [ppm] | Intensity [rel] |
| 1 | 162.8 | 28.2 |
| 2 | 151.7 | 21.3 |
| 3 | 150.7 | 23.7 |
| 4 | 139.1 | 15.5 |
| 5 | 138.0 | 15.8 |
| 6 | 127.4 | 22.8 |
| 7 | 126.9 | 25.1 |
| 8 | 124.3 | 6.0 |
| 9 | 120.4 | 26.1 |
| 10 | 117.7 | 28.4 |
| 11 | 78.7 | 26.4 |
| 12 | 77.9 | 49.0 |
| 13 | 72.6 | 39.7 |
| 14 | 33.4 | 43.9 |
| 15 | 25.9 | 12.2 |
| 16 | 21.7 | 14.4 |
| 17 | 20.0 | 100.0 |
| 18 | 18.8 | 24.5 |
| 19 | 17.9 | 46.2 |

### D. Solid-State ¹⁹F NMR

The ¹⁹F SSNMR of crystalline Compound I ethanol solvate was acquired using the procedure described in the General SSNMR Method. The ¹⁹F SSNMR spectrum for crystalline Compound I ethanol solvate is provided in FIG. 48, and the data are summarized below in Table 41.

TABLE 41
¹⁹F SSNMR Signals for Crystalline Compound I Ethanol Solvate

| | Chem Shift | |
|---|---|---|
| Peak # | [ppm] | Intensity [rel] |
| 1 | −63.1 | 11.6 |
| 2 | −64.2 | 12.5 |
| 3 | −78.0 | 4.7 |

### Example 16: Crystalline Compound I Solvate/Hydrate (Dry)

### A. Preparation of Crystalline Compound I Solvate/Hydrate (Dry)

Method 1: Crystalline Compound I heptane solvate Form A was stirred in water at room temperature for 2 weeks. Then it was filtered and air-dried to yield crystalline Compound I solvate/hydrate (dry).

Method 2: Crystalline Compound I neat amorphous form was dissolved in ethanol completely. Then water was added (water/ethanol=1.23-3.15). The system was stirred at 60° C. for 3 days. The solid phase was filtered and air-dried to yield crystalline Compound I solvate/hydrate (dry).

### B. X-Ray Powder Diffraction

The XRPD pattern for crystalline Compound I solvate/hydrate (dry) was recorded using the procedure described in the General XRPD Method.

The XRPD diffractogram for crystalline Compound I solvate/hydrate (dry) is provided in FIG. 49, and the XRPD data are summarized below in Table 42.

TABLE 42
XRPD Signals for Crystalline Compound I Solvate/Hydrate (dry)

| | Angle (degrees | |
|---|---|---|
| XRPD Peak No. | 2-Theta ± 0.2) | Intensity % |
| 1 | 4.4 | 17.6 |
| 2 | 8.8 | 100.0 |
| 3 | 10.3 | 41.9 |
| 4 | 11.3 | 17.5 |
| 5 | 11.7 | 21.6 |
| 6 | 13.4 | 10.4 |
| 7 | 14.1 | 23.4 |
| 8 | 15.1 | 13.0 |
| 9 | 17.7 | 83.4 |
| 10 | 18.1 | 13.9 |
| 11 | 18.9 | 23.5 |
| 12 | 20.6 | 31.0 |
| 13 | 21.2 | 52.7 |
| 14 | 22.3 | 28.5 |
| 15 | 22.7 | 10.8 |
| 16 | 22.9 | 14.3 |
| 17 | 23.3 | 14.0 |
| 18 | 27.0 | 13.1 |
| 19 | 28.3 | 10.8 |

### C. Thermogravimetric Analysis

TGA data for crystalline Compound I solvate/hydrate (dry) was collected on a Mettler Toledo TGA/DSC 3+ STARe System.

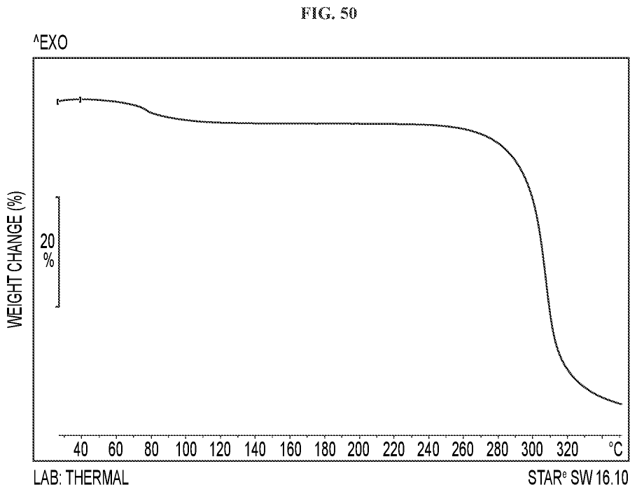

The TGA curve for crystalline Compound I solvate/hydrate (dry) is provided in FIG. 50. The thermogram showed ˜4.3% of weight loss from ambient temperature up to 100° C.

### D. Differential Scanning Calorimetry Analysis

The melting point of crystalline Compound I solvate/hydrate (dry) was measured using a Mettler Toledo TGA/DSC 3+ STARe System.

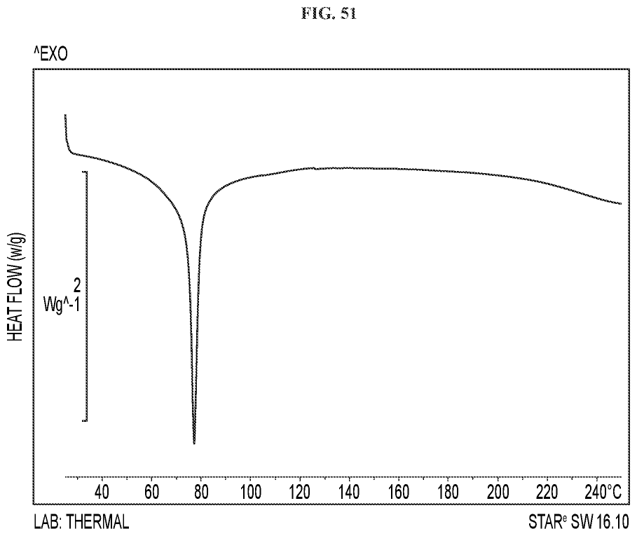

The DSC thermogram for crystalline Compound I solvate/hydrate (dry) is provided in FIG. 51. The thermogram shows an endotherm at −77° C.

### Example 17: Crystalline Compound I Solvate/Hydrate (Wet)

### A. Preparation of Crystalline Compound I Solvate/Hydrate (Wet)

Approximately 50 mg of crystalline Compound I hemihydrate Form C was weighed into a HPLC vial and 200 μl of ethanol/water 50:50 (% V/V), aw=0.83 was added. The mixture was stirred with a magnetic stirrer and kept in a cold room at 5° C. to yield crystalline Compound I solvate/hydrate (wet).

### B. X-Ray Powder Diffraction

The XRPD pattern for crystalline Compound I solvate/hydrate (wet) was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.01313030 and 49 s per step.

The XRPD diffractogram for crystalline Compound I solvate/hydrate (wet) is provided in FIG. 52, and the XRPD data are summarized below in Table 43.

TABLE 43
XRPD Signals for Crystalline Compound I Solvate/Hydrate (wet)

| | Angle (degrees | |
|---|---|---|
| XRPD Peak No. | 2-Theta ± 0.2) | Intensity % |
| 1 | 21.1 | 100.0 |
| 2 | 17.6 | 58.8 |
| 3 | 10.2 | 56.6 |
| 4 | 8.7 | 49.4 |
| 5 | 14.1 | 35.3 |
| 6 | 18.1 | 29.9 |
| 7 | 20.9 | 29.1 |
| 8 | 11.3 | 27.9 |
| 9 | 11.7 | 27.8 |
| 10 | 22.3 | 27.0 |
| 11 | 4.4 | 22.6 |
| 12 | 20.4 | 22.4 |
| 13 | 18.8 | 20.3 |
| 14 | 23.0 | 16.0 |
| 15 | 27.0 | 14.3 |
| 16 | 28.3 | 13.5 |
| 17 | 15.0 | 12.0 |
| 18 | 22.1 | 11.6 |
| 19 | 26.4 | 11.6 |
| 20 | 13.4 | 10.7 |
| 21 | 19.0 | 10.1 |

### C. Solid-State ¹³C NMR

The ¹³C SSNMR of crystalline Compound I solvate/hydrate (wet) was acquired using the procedure described in the General SSNMR Method. The ¹³C SSNMR spectrum for crystalline Compound I solvate/hydrate (wet) is provided in FIG. 53, and the data are summarized below in Table 44.

TABLE 44
¹³C SSNMR Signals for Crystalline
Compound I Solvate/Hydrate (wet)

| | Chem Shift | |
|---|---|---|
| Peak # | [ppm] | Intensity [rel] |
| 1 | 163.5 | 25.8 |
| 2 | 162.4 | 13.4 |
| 3 | 151.7 | 29.6 |
| 4 | 139.2 | 13.0 |
| 5 | 137.8 | 8.6 |
| 6 | 128.3 | 15.8 |
| 7 | 126.4 | 22.5 |
| 8 | 124.4 | 5.9 |
| 9 | 122.2 | 20.9 |
| 10 | 118.4 | 18.0 |
| 11 | 116.8 | 14.5 |
| 12 | 77.8 | 63.7 |
| 13 | 77.6 | 70.5 |
| 14 | 72.9 | 34.5 |
| 15 | 72.5 | 29.8 |
| 16 | 36.9 | 46.0 |
| 17 | 35.6 | 31.4 |
| 18 | 33.9 | 66.4 |
| 19 | 25.6 | 47.6 |
| 20 | 25.2 | 51.0 |
| 21 | 22.5 | 46.5 |
| 22 | 21.0 | 100.0 |
| 23 | 20.0 | 77.7 |
| 24 | 17.2 | 8.2 |

### D. Solid-State ¹⁹F NMR

The ¹⁹F SSNMR of crystalline Compound I solvate/hydrate (wet) was acquired using the procedure described in the General SSNMR Method. The ¹⁹F SSNMR spectrum for crystalline Compound I solvate/hydrate (wet) is provided in FIG. 54, and the data are summarized below in Table 45.

TABLE 45
¹⁹F SSNMR Signals for Crystalline
Compound I Solvate/Hydrate (wet)

| | Chem Shift | |
|---|---|---|
| Peak # | [ppm] | Intensity [rel] |
| 1 | −62.3 | 12.5 |
| 2 | −64.5 | 7.1 |
| 3 | −76.1 | 5.4 |
| 4 | −78.2 | 3.6 |

### Example 18: Crystalline Compound I L-Lysine Cocrystal

### A. Preparation of Crystalline Compound I L-Lysine Cocrystal

A mixture of ethanol and water at ratio of 30.8% to 69.2% by volume was saturated with L-lysine anhydrate. The mixture was then saturated with crystalline Compound I hemihydrate Form C. Then a 1:1 molar ratio of crystalline Compound I hemihydrate Form C to L-lysine (50 mg to 16.6 mg) was added to make a slurry. The slurry was mixed for 2 days, then sonicated and mixed on stir plate for additional 3 hours. The solids were isolated to yield crystalline Compound I L-lysine cocrystal.

### B. X-Ray Powder Diffraction

The XRPD diffractogram of crystalline Compound I L-lysine cocrystal was acquired at room temperature in transmission mode using a PANalytical Empyrean system equipped with a sealed tube source and a PIXcel 1D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I L-lysine cocrystal is provided in FIG. 55, and the XRPD data are summarized below in Table 46.

TABLE 46
XRPD Signals for Crystalline Compound I L-Lysine Cocrystal

| | Angle (degrees | |
|---|---|---|
| XRPD Peak No. | 2-Theta ± 0.2) | Intensity % |
| 1 | 21.6 | 100.0 |
| 2 | 21.1 | 77.4 |
| 3 | 10.5 | 69.7 |
| 4 | 7.9 | 69.3 |
| 5 | 19.9 | 60.4 |
| 6 | 17.8 | 47.1 |
| 7 | 9.5 | 45.8 |
| 8 | 20.8 | 43.4 |
| 9 | 11.4 | 43.0 |
| 10 | 22.9 | 41.6 |
| 11 | 18.2 | 39.7 |
| 12 | 17.5 | 36.8 |
| 13 | 22.3 | 30.8 |
| 14 | 3.9 | 24.9 |
| 15 | 11.8 | 24.5 |
| 16 | 8.9 | 24.2 |
| 17 | 19.2 | 22.7 |
| 18 | 29.7 | 22.1 |
| 19 | 13.8 | 21.9 |
| 20 | 26.6 | 21.8 |
| 21 | 13.4 | 21.0 |
| 22 | 27.0 | 20.6 |
| 23 | 13.3 | 18.2 |
| 24 | 16.4 | 16.8 |
| 25 | 26.3 | 14.2 |
| 26 | 15.9 | 12.9 |
| 27 | 15.4 | 11.5 |
| 28 | 27.5 | 11.5 |
| 29 | 29.2 | 10.4 |
| 30 | 23.6 | 10.3 |
| 31 | 18.6 | 10.2 |

### C. Thermogravimetric Analysis

The TGA of crystalline Compound I L-lysine cocrystal was measured using a TA Discovery 550 TGA from TA Instrument. A sample with a weight of approximately 1-10 mg was scanned from 25° C. to 300° C. at a heating rate of 10° C./min with a nitrogen purge.

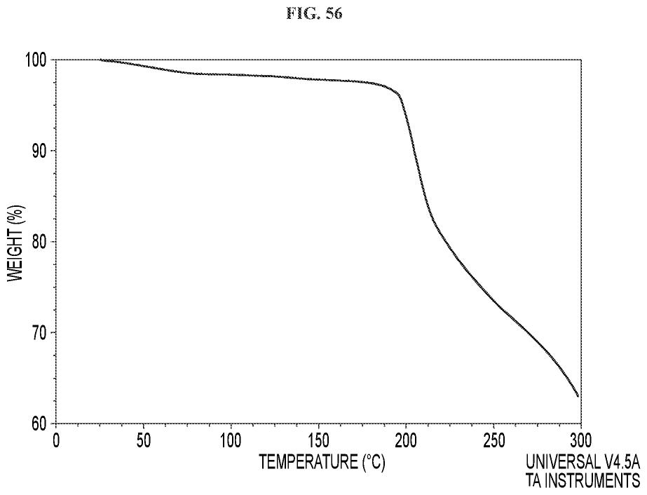

The TGA curve for crystalline Compound I L-lysine cocrystal is provided in FIG. 56. The thermogram showed gradual 1.6% weight loss from ambient temperature (23-25° C.) to 100° C.

### D. Differential Scanning Calorimetry Analysis

The DSC of VX crystalline Compound I L-lysine cocrystal was measured using a TA Instruments Q2000 DSC. A sample of between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to modulate at 0.320 every 60 seconds, then ramped at a rate of 10° C./min to a temperature of 300° C.

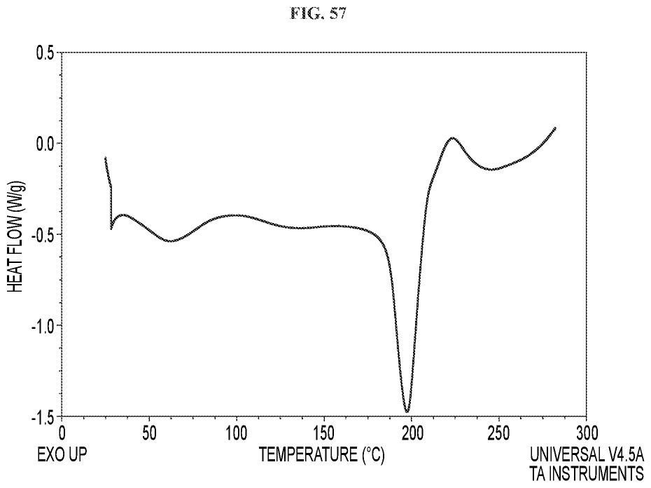

The DSC thermogram for crystalline Compound I L-lysine cocrystal is provided in FIG. 57. The thermogram showed endotherms at 62° C. and 198° C.

### E. Solid-State ¹³C NMR

The ¹³C SSNMR of crystalline Compound I L-lysine cocrystal was acquired using the procedure described in the General SSNMR Method. The ¹³C SSNMR spectrum for crystalline Compound I L-lysine cocrystal is provided in FIG. 58, and the data are summarized below in Table 47.

TABLE 47
¹³C SSNMR Signals for Crystalline
Compound I L-Lysine Cocrystal

| | Chem Shift | |
|---|---|---|
| Peak # | [ppm] | Intensity [rel] |
| 1 | 181.6 | 21.9 |
| 2 | 180.9 | 21.9 |
| 3 | 177.5 | 23.1 |
| 4 | 165.4 | 13.0 |
| 5 | 164.4 | 21.1 |
| 6 | 163.7 | 38.4 |
| 7 | 162.7 | 26.5 |
| 8 | 151.9 | 48.2 |
| 9 | 150.7 | 17.3 |
| 10 | 138.9 | 25.1 |
| 11 | 138.2 | 21.5 |
| 12 | 127.6 | 36.9 |
| 13 | 126.8 | 26.5 |
| 14 | 125.8 | 24.4 |
| 15 | 124.1 | 12.9 |
| 16 | 121.4 | 16.9 |
| 17 | 119.6 | 23.7 |
| 18 | 118.0 | 31.8 |
| 19 | 78.4 | 18.7 |
| 20 | 77.1 | 48.5 |
| 21 | 75.9 | 15.6 |
| 22 | 73.1 | 83.1 |
| 23 | 56.8 | 43.0 |
| 24 | 54.9 | 25.6 |
| 25 | 45.1 | 10.0 |
| 26 | 43.6 | 14.7 |
| 27 | 41.4 | 38.4 |
| 28 | 39.6 | 33.4 |
| 29 | 38.8 | 18.6 |
| 30 | 37.0 | 36.0 |
| 31 | 34.3 | 66.4 |
| 32 | 33.4 | 75.1 |
| 33 | 32.2 | 41.4 |
| 34 | 31.6 | 52.7 |
| 35 | 30.6 | 59.7 |
| 36 | 29.2 | 22.9 |
| 37 | 27.4 | 55.8 |
| 38 | 25.8 | 51.1 |
| 39 | 25.1 | 71.3 |
| 40 | 22.9 | 35.55 |
| 41 | 22.5 | 34.4 |
| 42 | 21.7 | 27.55 |
| 43 | 20.5 | 64.2 |
| 44 | 19.4 | 100.0 |
| 45 | 18.6 | 98.2 |

### Example 18: Crystalline Compound I L-Arginine Cocrystal

### A. Preparation of Crystalline Compound I L-Arginine Cocrystal

A solvent system of ethanol and water at ratio of 30.8% to 69.2% by volume was prepared. A 1:1 molar ratio of crystalline Compound I hemihydrate Form C and L-arginine (˜10 mg to ˜3.9 mg) was added to a 2 ml ball mill vial with 2.8 mm ceramic (zirconium oxide) beads and 2 μL of the ethanol/water solvent system. The vial was placed in the high throughput ball mill and run at 7500 RPM for 60 seconds with 10 second pauses for 5 cycles. The solid was placed in a vacuum oven at 45° C. overnight to yield crystalline Compound I L-arginine cocrystal.

### B. X-Ray Powder Diffraction

The XRPD diffractogram of crystalline Compound I L-arginine cocrystal was acquired at room temperature (23-25° C.) in transmission mode using a PANalytical Empyrean system equipped with a sealed tube source and a PIXcel 3D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I L-arginine cocrystal is provided in FIG. 59, and the XRPD data are summarized below in Table 48.

TABLE 48
XRPD Signals for Crystalline Compound I L-Arginine Cocrystal

| | Angle (degrees | |
|---|---|---|
| XRPD Peak No. | 2-Theta ± 0.2) | Intensity % |
| 1 | 23.1 | 100.0 |
| 2 | 18.3 | 83.2 |
| 3 | 19.4 | 68.9 |
| 4 | 19.1 | 64.5 |
| 5 | 7.5 | 61.1 |
| 6 | 27.4 | 59.0 |
| 7 | 21.0 | 50.9 |
| 8 | 21.9 | 48.4 |
| 9 | 9.0 | 28.6 |
| 10 | 10.5 | 22.5 |
| 11 | 15.9 | 20.9 |
| 12 | 13.4 | 19.6 |

### C. Thermogravimetric Analysis

The TGA of crystalline Compound I L-arginine cocrystal was measured using TA Discovery 550 TGA from TA Instrument. Approximately 1-10 mg was scanned from 25° C. to 300° C. at a heating rate of 10° C./min with a nitrogen purge.

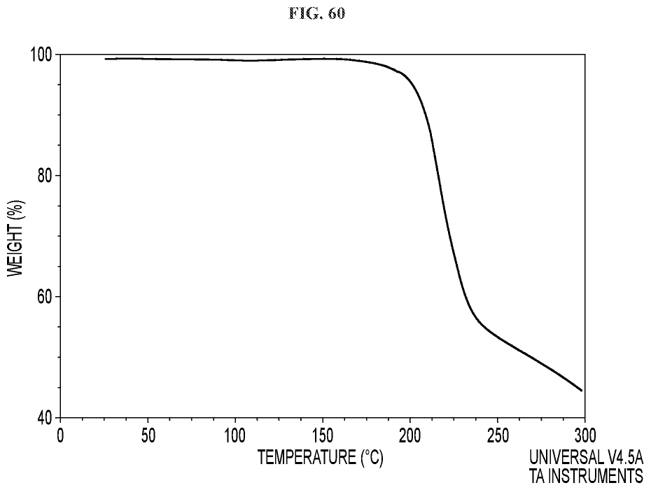

The TGA curve for crystalline Compound I L-arginine cocrystal is provided in FIG. 60. The thermogram showed little to no weight loss until degradation

### D. Differential Scanning Calorimetry Analysis

DSC of crystalline Compound I L-arginine cocrystal was measured using a TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to ramp from room temperature (23-25° C.) with a rate of 10° C./min to a temperature of 300° C.

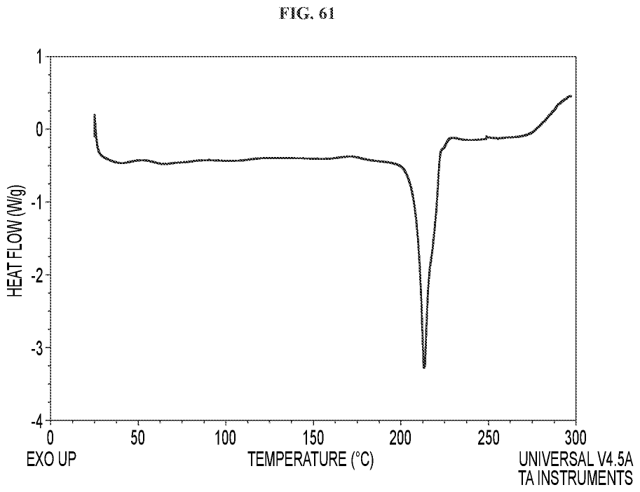

The DSC thermogram for crystalline Compound I L-arginine cocrystal is provided in FIG. 61. The thermogram showed an endotherm at 209° C.

### Example 19: Crystalline Compound I L-Phenylalanine Cocrystal

### A. Preparation of Crystalline Compound I L-Phenylalanine Cocrystal

A solvent system of ethanol and water at ratio of 30.8% to 69.2% by volume was prepared. A 1:1 molar ratio of crystalline Compound I hemihydrate Form C and L-phenylalanine (˜10 mg to ˜3.7 mg) was added to a 2 ml ball mill vial with 2.8 mm ceramic (zirconium oxide) beads and 2 μL of the ethanol/water solvent system. The vial was placed in the high throughput ball mill and run at 7500 RPM for 60 seconds with 10 second pauses for 5 cycles. The solid was placed in vacuum oven at 45° C. overnight to yield crystalline Compound I L-phenylalanine cocrystal.

### B. X-Ray Powder Diffraction

The XRPD diffractogram of crystalline Compound I L-phenylalanine cocrystal was acquired at room temperature (23-25° C.) in transmission mode using a PANalytical Empyrean system equipped with a sealed tube source and a PIXcel 3D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I L-phenylalanine cocrystal is provided in FIG. 62, and the XRPD data are summarized below in Table 49.

TABLE 49
XRPD Signals for Crystalline Compound
I L-Phenylalanine Cocrystal

| | Angle (degrees | |
|---|---|---|
| XRPD Peak No. | 2-Theta ± 0.2) | Intensity % |
| 1 | 17.6 | 100.0 |
| 2 | 10.1 | 73.3 |
| 3 | 20.5 | 57.8 |
| 4 | 6.5 | 52.2 |
| 5 | 11.1 | 42.3 |
| 6 | 19.8 | 41.2 |
| 7 | 14.8 | 39.5 |
| 8 | 15.3 | 34.3 |
| 9 | 18.4 | 32.0 |
| 10 | 21.4 | 28.6 |
| 11 | 26.3 | 26.4 |
| 12 | 22.9 | 26.4 |
| 13 | 7.4 | 24.6 |
| 14 | 23.9 | 21.7 |
| 15 | 4.9 | 21.2 |
| 16 | 9.0 | 20.8 |
| 17 | 27.9 | 19.9 |
| 18 | 16.2 | 17.4 |
| 19 | 22.2 | 14.5 |

### C. Differential Scanning Calorimetry Analysis

The DSC of crystalline Compound I L-phenylalanine cocrystal was measured using a TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to ramp from room temperature (23-25° C.) with a rate of 10° C./min to a temperature of 300° C.

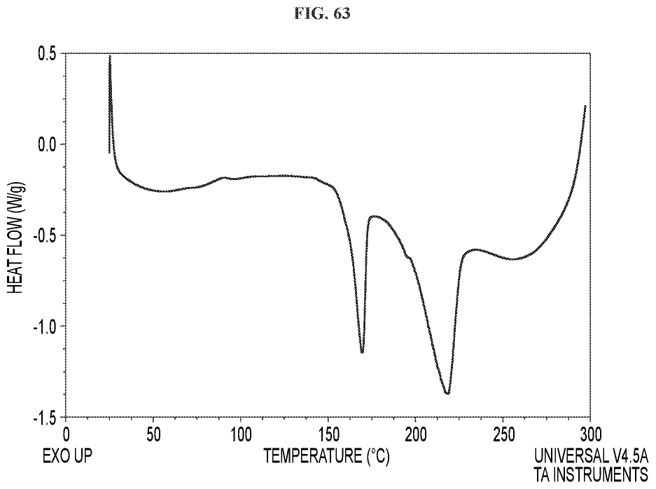

The DSC thermogram for crystalline Compound I L-phenylalanine cocrystal is provided in FIG. 63. The thermogram showed endotherm peaks at 169 and 217° C.

### Example 20: Crystalline Compound I Succinic Acid Cocrystal (Wet)

### A. Preparation of Crystalline Compound I Succinic Acid Cocrystal (Wet)

A solvent system of ethanol and water at ratio of 30.8% to 69.2% by volume was prepared. A 1:1 molar ratio of crystalline Compound I hemihydrate Form C and succinic acid (˜10 mg to ˜3.2 mg) was added to a 2 ml ball mill vial with 2.8 mm ceramic (zirconium oxide) beads and 2 μL of the ethanol/water solvent system. The vial was placed in the high throughput ball mill and run at 7500 RPM for 60 seconds with 10 second pauses for 5 cycles. The solid was placed in vacuum oven at 45° C. overnight, then placed in a humidity chamber at 40° C., 75% Relative Humidity to yield crystalline Compound I succinic acid cocrystal hydrate (wet).

### B. X-Ray Powder Diffraction

The XRPD diffractogram of crystalline Compound I succinic acid cocrystal (wet) was acquired at room temperature (23-25° C.) in transmission mode using a PANalytical Empyrean system equipped with a sealed tube source and a PIXcel 3D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I succinic acid cocrystal (wet) is provided in FIG. 64, and the XRPD data are summarized below in Table 50.

TABLE 50
XRPD Signals for Crystalline Compound
I Succinic Acid Cocrystal (wet)

| | Angle (degrees | |
|---|---|---|
| XRPD Peak No. | 2-Theta ± 0.2) | Intensity % |
| 1 | 4.0 | 100.0 |
| 2 | 21.7 | 86.9 |
| 3 | 22.0 | 43.1 |
| 4 | 13.5 | 40.8 |
| 5 | 14.4 | 36.3 |
| 6 | 27.1 | 30.2 |
| 7 | 20.4 | 29.7 |
| 8 | 9.1 | 28.7 |
| 9 | 12.1 | 27.1 |
| 10 | 8.1 | 25.8 |
| 11 | 22.7 | 21.7 |
| 12 | 16.8 | 20.9 |
| 13 | 8.9 | 19.9 |
| 14 | 17.9 | 15.5 |
| 15 | 20.1 | 14.7 |
| 16 | 26.1 | 14.0 |
| 17 | 9.8 | 12.0 |
| 18 | 28.0 | 10.5 |

### Example 21: Crystalline Compound I Succinic Acid Cocrystal (Dry)

### A. Preparation of Crystalline Compound I Succinic Acid Cocrystal (Dry)

A solvent system of ethanol and water at ratio of 30.8% to 69.2% by volume was prepared. A 1:1 molar ratio of crystalline Compound I hemihydrate Form C and succinic acid (˜10 mg to ˜3.2 mg) was added to a 2 ml ball mill vial with 2.8 mm ceramic (zirconium oxide) beads and 2 μL of the ethanol/water solvent system. The vial was placed in the high throughput ball mill and run at 7500 RPM for 60 seconds with 10 second pauses for 5 cycles. The solid was placed in vacuum oven at 45° C. overnight to yield crystalline Compound I succinic acid cocrystal (dry).

### B. X-Ray Powder Diffraction

The XRPD diffractogram of crystalline Compound I succinic acid cocrystal (dry) was acquired at room temperature (23-25° C.) in transmission mode using a PANalytical Empyrean system equipped with a sealed tube source and a PIXcel 3D Medipix-3 detector (Malvern PANalytical Inc, Westborough, Massachusetts). The X-ray generator was operated at a voltage of 45 kV and a current of 40 mA with copper radiation (1.54060 Å). The powder sample was placed on a 96 well sample holder with mylar film and loaded into the instrument. The sample was scanned over the range of about 3 to about 40 degrees two-theta with a step size of 0.0131303° and 49 s per step.

The XRPD diffractogram for crystalline Compound I succinic acid cocrystal (dry) is provided in FIG. 65, and the XRPD data are summarized below in Table 51.

TABLE 51
XRPD Signals for Crystalline Compound
I Succinic Acid Cocrystal (dry)

| | Angle (degrees | |
|---|---|---|
| XRPD Peak No. | 2-Theta ± 0.2) | Intensity % |
| 1 | 22.0 | 100.0 |
| 2 | 4.1 | 60.0 |
| 3 | 27.1 | 38.1 |
| 4 | 26.1 | 30.9 |
| 5 | 20.0 | 21.2 |
| 6 | 8.2 | 14.4 |
| 7 | 25.5 | 11.0 |

### C. Differential Scanning Calorimetry Analysis

The DSC of crystalline Compound I succinic acid cocrystal (dry) was measured using a TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to ramp from room temperature (23-25° C.) with a rate of 10° C./min to a temperature of 300° C.

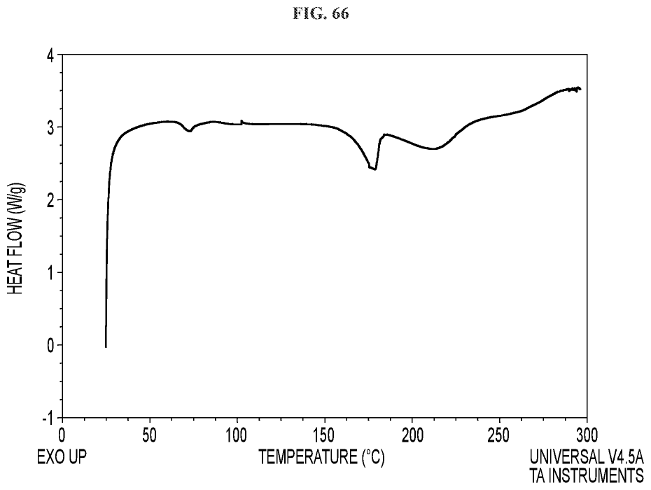

The DSC thermogram for crystalline Compound I succinic acid cocrystal (dry) is provided in FIG. 66. The thermogram showed endotherm peaks at 73, 179, and 214° C.

### Example 22: Crystalline Compound I Methanol Solvate/Hydrate

### A. Preparation of Crystalline Compound I Methanol Solvate/Hydrate

Approximately 10 mg of Compound I hemihydrate Form C was stirred in 5 μl of methanol with a spatula to yield crystalline Compound I methanol solvate/hydrate.

### B. X-Ray Powder Diffraction

The XRPD pattern for crystalline Compound I methanol solvate/hydrate was acquired at room temperature in reflection mode using a Bruker Advance equipped with Vantec-1 detector. The sample was analyzed on a silicon sample holder from 3-40° 2-theta on continuous mode with step size of 0.0144531° and time per step of 0.25 seconds. The sample was spinning at 15 rpm.

The XRPD diffractogram for crystalline Compound I methanol solvate/hydrate is provided in FIG. 67, and the XRPD data are summarized below in Table 52.

TABLE 52
XRPD Signals for Crystalline Compound
I Methanol Solvate/Hydrate

| | Angle (degrees | |
|---|---|---|
| XRPD Peak No. | 2-Theta ± 0.2) | Intensity % |
| 1 | 8.2 | 100.0 |
| 2 | 16.4 | 90.7 |
| 3 | 20.5 | 74.7 |
| 4 | 21.5 | 32.2 |
| 5 | 18.7 | 22.0 |
| 6 | 10.8 | 21.3 |
| 7 | 18.5 | 15.1 |
| 8 | 17.9 | 12.7 |
| 9 | 8.8 | 12.1 |
| 10 | 14.3 | 11.4 |
| 11 | 20.0 | 10.7 |
| 12 | 26.9 | 10.6 |

### C. Solid-State ¹³C NMR

The ¹³C SSNMR of crystalline Compound I methanol solvate/hydrate was acquired using the procedure described in the General SSNMR Method. The ¹³C SSNMR spectrum for crystalline Compound I methanol solvate/hydrate is provided in FIG. 68, and the data are summarized below in Table 53.

TABLE 53
¹³C SSNMR Signals for Crystalline
Compound I Methanol Solvate/Hydrate

| | Chem Shift | |
|---|---|---|
| Peak # | [ppm] | Intensity [rel] |
| 1 | 163.3 | 25.0 |
| 2 | 162.2 | 20.3 |
| 3 | 151.6 | 28.8 |
| 4 | 150.8 | 31.8 |
| 5 | 138.4 | 32.8 |
| 6 | 126.4 | 38.1 |
| 7 | 125.4 | 21.3 |
| 8 | 122.3 | 12.2 |
| 9 | 121.5 | 10.4 |
| 10 | 120.7 | 21.8 |
| 11 | 118.9 | 13.6 |
| 12 | 118.2 | 32.7 |
| 13 | 117.4 | 17.6 |
| 14 | 77.5 | 95.3 |
| 15 | 73.2 | 50.1 |
| 16 | 49.4 | 6.6 |
| 17 | 36.5 | 32.3 |
| 18 | 35.2 | 51.9 |
| 19 | 34.1 | 30.1 |
| 20 | 33.5 | 53.8 |
| 21 | 32.5 | 39.4 |
| 22 | 25.4 | 66.9 |
| 23 | 22.8 | 32.0 |
| 24 | 22.1 | 37.6 |
| 25 | 21.4 | 66.2 |
| 26 | 20.5 | 100.0 |
| 27 | 20.0 | 61.1 |
| 28 | 19.5 | 65.2 |

### D. Solid-State ¹⁹F NMR

The ¹⁹F SSNMR of crystalline Compound I methanol solvate/hydrate was acquired using the procedure described in the General SSNMR Method. The ¹⁹F SSNMR spectrum for crystalline Compound I methanol solvate/hydrate is provided in FIG. 69, and the data are summarized below in Table 54.

TABLE 54
¹⁹F SSNMR Signals for Crystalline
Compound I Methanol Solvate/Hydrate

| | Chem Shift | |
|---|---|---|
| Peak # | [ppm] | Intensity [rel] |
| 1 | −64.0 | 12.3 |
| 2 | −64.6 | 12.5 |
| 3 | −79.0 | 1.9 |

### E. Single Crystal X-Ray Diffraction

X-ray diffraction data for crystalline Compound I methanol solvate/hydrate were acquired at 100K on a Bruker diffractometer equipped with Cu Kα radiation (λ=1.54178 Å) and a CPAD detector. The structure was solved and refined using SHELX programs (Sheldrick, G. M., Acta Cryst., (2008) A64, 112-122) and results are summarized in Table 55 below.

TABLE 55
Single Crystal Elucidation of Crystalline
Compound I Methanol Solvate/Hydrate

| Crystal System | Monoclinic |
|---|---|
| Space Group | C₂ |
| a (Å) | 12.7248(11) |
| b (Å) | 15.7052(14) |
| c (Å) | 42.958(4) |
| α (°) | 90 |
| β (°) | 90.893(3) |
| γ (°) | 90 |
| V (Å3) | 8583.8(13) |
| Z/Z′ | 2/4 |
| Temperature (K) | 100(2) |
| Density (g/cm³) | 1.479 |

### Synthetic Examples

All the specific and generic compounds, and the intermediates disclosed for making those compounds, are considered to be part of the disclosure.

One of ordinary skill in the art will appreciate that the methods described herein are generally applicable to other stereoisomers of the compounds described herein.

Should the name of a compound conflict with the structure of the compound anywhere in the present application, the structure supersedes the name and is intended to be controlling.

### Example 23: Synthesis of Bis-amide Precursors

### Intermediate 1: Preparation of 6-(Benzyloxy)-3-((tert-butoxycarbonyl)amino)-5-(trifluoromethyl)picolinic acid (Compound 17)

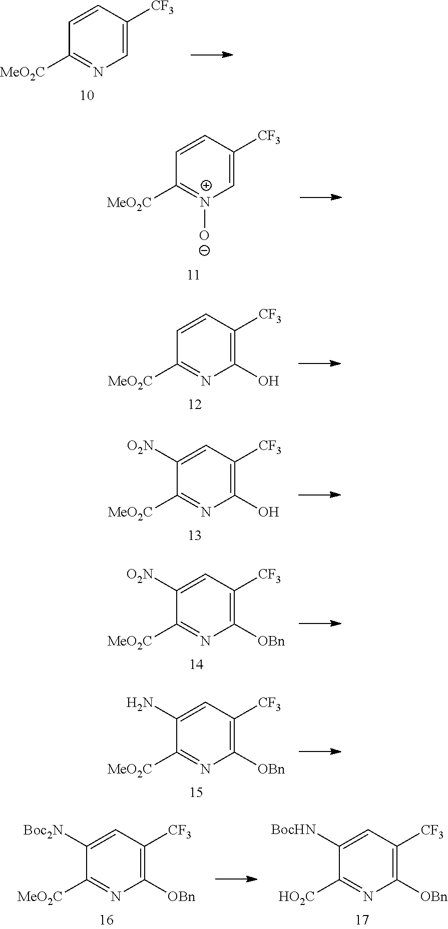

### Step 1: Methyl 1-oxido-5-(trifluoromethyl)pyridin-1-ium-2-carboxylate (11)

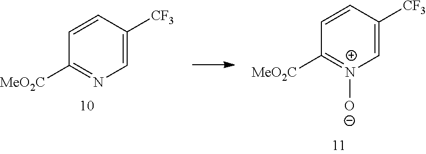 [SMILES: COC(=O)c1ccc(C(F)(F)F)cn1 ; COC(=O)c1ccc(C(F)(F)F)c[n+]1[O-]]

Urea hydrogen peroxide (62.7 g, 646.53 mmol) was added portion-wise to a stirred solution of methyl 5-(trifluoromethyl)pyridine-2-carboxylate (40 g, 191.09 mmol) in 1,2-dichloroethane (300 mL) at 0° C. Trifluoroacetic anhydride (107.70 g, 72 mL, 507.65 mmol) was then added over 30 minutes at a temperature of −10° C., with cooling bath (CO₂/acetone bath). The reaction mixture was then stirred for a further 30 minutes at a temperature of 0° C. and then for 1 hour at ambient temperature. The reaction mixture was then poured into cooled ice-water (600 mL). The mixture was diluted with dichloromethane (300 mL) and then layers were separated. The aqueous phase was extracted with dichloromethane (2×200 mL). The combined organic phase was washed with water (2×300 mL) and brine (1×200 mL), dried over anhydrous sodium sulfate, filtered and concentrated under reduced pressure to give methyl 1-oxido-5-(trifluoromethyl)pyridin-1-ium-2-carboxylate (47.6 g, 90%) as light yellow solid. ¹H NMR (300 MHz, DMSO-d₆) δ 8.89 (s, 1H), 8.02-7.90 (m, 1H), 7.86-7.72 (m, 1H), 3.89 (s, 3H) ppm. ¹⁹F NMR (282 MHz, DMSO-d₆) δ −62.00 (s, 3F) ppm. ESI-MS m z calc. 221.02998, found 222.1 (M+1)⁺; Retention time: 1.24 minutes. LCMS Method: Kinetex Polar C₁₈ 3.0×50 mm 2.6 μm, 3 min, 5-95% acetonitrile in H₂O (0.1% formic acid) 1.2 mL/min.

### Step 2: Methyl 6-hydroxy-5-(trifluoromethyl)pyridine-2-carboxylate (12)

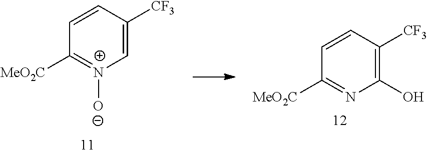 [SMILES: COC(=O)c1ccc(C(F)(F)F)c[n+]1[O-] ; COC(=O)c1ccc(C(F)(F)F)c(O)n1]

Trifluoroacetic anhydride (291.62 g, 193 mL, 1.3885 mol) was added dropwise to a mixture of methyl 1-oxido-5-(trifluoromethyl)pyridin-1-ium-2-carboxylate (51.058 g, 230.66 mmol) in DMF (305 mL) at 0° C. The mixture was then stirred at room temperature overnight. The mixture was concentrated under reduced pressure to remove excess of trifluoroacetic acid. The residual DMF solution was poured dropwise to a 0° C. cooled and stirring water volume (1000 mL). The precipitated solid was collected by filtration and then washed with water (300 mL). The solid was dried over high vacuum to afford methyl 6-hydroxy-5-(trifluoromethyl)pyridine-2-carboxylate (45.24 g, 86%) as white solid. ¹H NMR (300 MHz, DMSO-d₆) δ 7.90 (d, J=7.2 Hz, 1H), 7.03 (d, J=7.2 Hz, 1H), 4.02 (s, 3H) ppm. ¹⁹F NMR (282 MHz, DMSO-d₆) δ −66.39 (s, 3F) ppm. ESI-MS m/z calc. 221.03, found 222.1 (M+1)+; Retention time: 1.43 minutes. LCMS Method: Kinetex Polar C18 3.0×50 mm 2.6 μm, 3 min, 5-95% acetonitrile in H₂O (0.1% formic acid) 1.2 mL/min.

### Step 3: Methyl 6-hydroxy-3-nitro-5-(trifluoromethyl)pyridine-2-carboxylate (13)

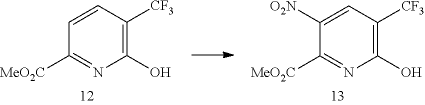 [SMILES: COC(=O)c1ccc(C(F)(F)F)c(O)n1 ; COC(=O)c1nc(O)c(C(F)(F)F)cc1[N+](=O)[O-]]

To an ice-cooled solution of methyl 6-hydroxy-5-(trifluoromethyl)pyridine-2-carboxylate (33.04 g, 149.41 mmol) in sulfuric acid (200 mL of 18.4 M, 3.6800 mol) was added nitric acid (13 mL of 15.8 M, 205.40 mmol) dropwise. After 5 min, the ice bath was removed, and the reaction mixture was stirred at 38° C. overnight. The reaction was not completed, nitric acid (3 mL of 15.8 M, 47.400 mmol) was added dropwise at room temperature and the reaction was heated at 38° C. for 4.5 hours. The reaction was poured slowly into ice-cold water (900 mL) and the mixture was cooled at 0° C. for 15 minutes. Then the resultant solid was isolated by filtration and washed with water (600 mL). The solid was dried overnight under high vacuum to give methyl 6-hydroxy-3-nitro-5-(trifluoromethyl)pyridine-2-carboxylate (39.49 g, 99%) as white solid. ¹H NMR (300 MHz, DMSO-d₆) δ 8.54 (s, 1H), 3.95 (s, 3H) ppm. ¹⁹F NMR (282 MHz, DMSO-d₆) δ −64.56 (s, 3F) ppm. ESI-MS m/z calc. 266.0151, found 267.1 (M+1)⁺; Retention time: 1.64 minutes. LCMS Method: Kinetex Polar C18 3.0×50 mm 2.6 μm, 3 min, 5-95% acetonitrile in H₂O (0.1% formic acid) 1.2 mL/min.

### Step 4: Meth 16-(benzyloxy)-3-nitro-5-(trifluoromethyl)picolinate (14)

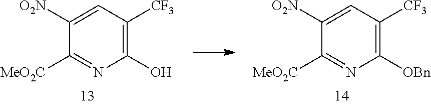 [SMILES: COC(=O)c1nc(O)c(C(F)(F)F)cc1[N+](=O)[O-] ; COC(=O)c1nc(OCc2ccccc2)c(C(F)(F)F)cc1[N+](=O)[O-]]

In a 1 L 3 necked RBF, charged with stirring bar, J-Kem temperature probe and a solution of methyl 6-hydroxy-3-nitro-5-(trifluoromethyl)pyridine-2-carboxylate (20 g, 75.151 mmol, 1 equiv.) in toluene (200 mL, 10 Vols) was added benzyl alcohol (8.939 g, 8.554 mL, 1.045 g/mL, 82.666 mmol, 1.1 equiv.) and triphenylphosphine (23.653 g, 90.181 mmol, 1.2 equiv.). The mixture was stirred at ambient for 30 min (there was still insoluble solid in the reaction mixture), cool down to 0-5° C., diisopropyl azodicarboxylate (20.056 g, 19.528 mL, 1.027 g/mL, 97.696 mmol, 1.3 equiv.) was added drop wise through addition funnel over 90 min, an exotherm was observed, maintain the reaction temp below 10° C. during the addition. During the addition, the mixture became clear then turned cloudy. After addition, the ice bath was removed, allowed the reaction warm to ambient. After 1 h, the reaction was complete by LC analysis. Added 200 mL of heptane, cooled down with ice bath, stirred, slowly warm to ambient overnight. The precipitate was filtered off (UPLC checked, no product), the filtrate was concentrated and purified by column chromatography (330 g silica gel) eluting with EtOAc/hexane 0-10% to give two fractions. Non-polar fractions were concentrated and triturated with heptane, the obtained solid was collected by filtration, dried under vacuum oven at 40° C. with N₂ bleed overnight to afford 11.16 g of product as white solid. The filtrate was concentrated and triturated with heptane, the obtained solid was collected by filtration, dried under vacuum oven at 40° C. with N₂ bleed overnight to afford 0.5 g of product as a 2nd crop. Combined crops yield 11.66 g of methyl 6-hydroxy-3-nitro-5-(trifluoromethyl)picolinate. ¹H NMR (400 MHz, DMSO-d₆) δ 8.82 (s, 1H), 7.55-7.25 (m, 5H), 5.62 (s, 2H), 3.98 (s, 3H) ppm. ESI-MS m/z calc.
356.06, found 357.09 (M+1)⁺.

### Step 5: Methyl 3-amino-6-(benzyloxy)-5-(trifluoromethyl)picolinate (15)

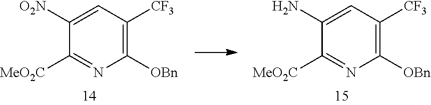 [SMILES: COC(=O)c1nc(OCc2ccccc2)c(C(F)(F)F)cc1[N+](=O)[O-] ; COC(=O)c1nc(OCc2ccccc2)c(C(F)(F)F)cc1N]

In a 500 mL 3 necked RBF, methyl 6-hydroxy-3-nitro-5-(trifluoromethyl)picolinate (10 g, 28.07 mmol, 1 equiv.) was stirred in THF (50 mL, 5 Vols)/EtOH (50 mL, 5 Vols) and cooled in in an ice bath. Na₂S₂O₄ (22.998 g, 112.278 mmol, 4 equiv., 85% w/w tech grade) in water (150 mL, 15 Vols) was added drop wise via addition funnel, keeping the temperature below 20° C. during course of addition. Ice bath was removed after complete addition (˜1 hr). After complete reaction by LC analysis, 100 mL of 2-MeTHF/50 mL of water added. Organic layer washed with 40 mL of 1:1 water/brine. 70 mL of 1 M HCl added to organic layer. After stirring ˜30 min, the phases were separated. The organic layer was washed with 40 mL of 20% aq w/v KHCO₃ then dried (Na₂SO₄), filtered, and concentrated. 100 mL of heptane added and concentrated to a solid. Crude solid triturated with 70 mL of heptane. Stirred at ambient, the solid was collected by filtration and washed with heptane (2×20 mL), dried further in vacuum oven at 40° C. overnight with N₂ bleed. Obtained 8.84 g of a solid (yield 96.5%). ¹H NMR (400 MHz, DMSO) δ 7.75 (s, 1H), 7.50-7.27 (m, 5H), 6.63 (s, 2H), 5.35 (s, 2H), 3.86 (s, 3H) ppm. ESI-MS m/z calc. 326.09, found 327.03 (M+1)⁺.

### Step 6: Methyl 6-(benzyloxy)-3-(bis(tert-butoxycarbonyl)amino)-5-(trifluoromethyl)picolinate (16)

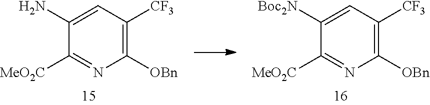

In 250 mL RBF, charged methyl 3-amino-6-(benzyloxy)-5-(trifluoromethyl)picolinate (7.6 g, 23.293 mmol, 1 equiv.), Boc₂O (15.251 g, 69.88 mmol, 3 equiv.) in 2-MeTHF (76 mL, 10 Vols), and DMAP (569.136 mg, 4.659 mmol, 0.2 equiv.) and mixture stirred at ambient temperature After complete reaction by LC analysis, added water (38 mL), stirred, phase split, aqueous layer was removed, the organic layer was washed with brine/water (1:1, 40 mL×2), dried over Na₂SO₄, filtered, concentrated to afford 14.64 g of methyl 6-(benzyloxy)-3-(bis(tert-butoxycarbonyl)amino)-5-(trifluoromethyl)picolinate as a viscous oil used as is for next step. ESI-MS m/z calc. 526.19, found 527.14 (M+1)⁺. ESI-MS m/z calc. 878.26, found 879.29 (M+1)⁺.

### Step 7: 6-(Benzyloxy)-3-((tert-butoxycarbonyl)amino)-5-(trifluoromethyl)picolinic acid (17)

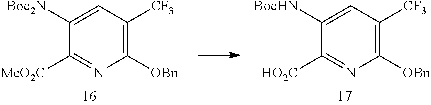

To a 500 mL RBF with a mixture of methyl 6-(benzyloxy)-3-(bis(tert-butoxycarbonyl)amino)-5-(trifluoromethyl)picolinate (12.264 g, 23.293 mmol, 1 equiv.) in THF (85.848 mL, 7 volumes) was added LiOH hydrate (9.775 g, 232.93 mmol, 10 equiv.) in water (60 mL, 4.892 volumes) through addition funnel and the mixture heated to 55° C. After complete reaction by LC analysis, the mixture was cooled to ambient temperature. Acidified with aq HCl (38.822 mL, 6 M, 232.93 mmol, 10 equiv.) to pH-2 (pH paper), the reaction mixture became clear. 2-MeTHF (90 mL) was added, stirred, and phase split. The organic layer was washed with brine/water (1:1, 40 mL), dried over Na₂SO₄, filtered, concentrated on rotary evaporator, swap solvent with heptane. The residue (solid) was triturated with heptane (70 mL), stirred at ambient overnight. The solid was collected by filtration, rinsed with heptane (7 mL×3), dried in vacuum oven at 40° C. with N₂ bleed overnight to afford 9.0 g of 6-(benzyloxy)-3-((tert-butoxycarbonyl)amino)-5-(trifluoromethyl)picolinic acid (89%). ¹H NMR (400 MHz, DMSO) δ 10.06 (s, 1H), 8.88 (s, 1H), 7.54-7.26 (m, 6H), 5.52 (s, 2H), 1.49 (s, 9H) ppm. ESI-MS m/z calc. 412.12, found 413.07 (M+1)⁺.

### Intermediate 2: Preparation of (2R,8S)-2-(Benzyloxy)-8-((tert-butyldiphenylsilyl)oxy)-2-(trifluoromethyl)nonanehydrazide (22)

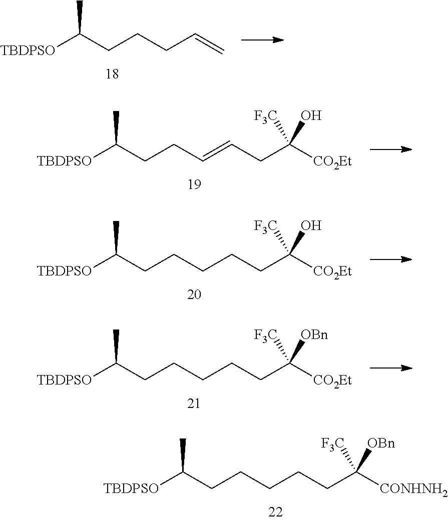

The synthesis of (R)-tert-butyl(hept-6-en-2-yloxy)diphenylsilane (18) is disclosed, for example, in Gaddam, et al. Org. Biomol. Chem., 2019, 17, 5601-5614.

### Step 1: Ethyl (2R,8S,E)-8-((tert-butyldiphenylsilyl)oxy)-2-hydroxy-2-(trifluoromethyl)non-4-enoate (19)

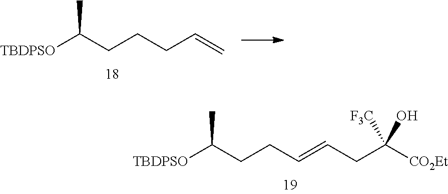

A solution of (S-BINAP)PdCl₂ (14.8 g, 18 mmol, 0.05 eq) and AgSbF₆ (12.7 g, 37 mmol, 0.1 eq) in DCM (1.3 L) was stirred under Ar at 30° C. for 1 hr (AgSbF₆ was moisture absorbed, so it was weighed in glove box). After that, a solution of (S)-tert-butyl(hept-6-en-2-yloxy)diphenylsilane (130 g, 369 mmol, 1 eq) and ethyl 3,3,3-trifluoro-2-oxo-propanoate (87 g, 509 mmol, 68 mL, 1.4 eq) in DCM (260 mL) was added to the mixture and stirred under argon gas at 30° C. for 15 hrs. TLC (petroleum ether:ethyl acetate=10:1, Rf=0.5) showed trace of (S)-tert-butyl(hept-6-en-2-yloxy)diphenylsilane remained and a new spot with larger polarity was formed. The mixture was filtered, and the filtrate was concentrated in vacuum. The residue was purified by silica gel column chromatography (ethyl acetate/petroleum ether=0/1 to 1/50) to obtain ethyl (2R,8S,E)-8-((tert-butyldiphenylsilyl)oxy)-2-hydroxy-2-(trifluoromethyl)non-4-enoate (133 g, 254 mmol, 69% yield, 99.5% purity). ¹H NMR (400 MHz, CDCl3) δ 7.6 (m, 4H), 7.4 (m, 6H), 5.5 (m, 1H), 5.2 (m, 1H), 4.3 (m, 2H), 3.8 (m, 1H), 2.6 (m, 2H), 2.0 (m, 2H), 1.4 (m, 2H), 1.3 (m, 3H), 1.0 (m, 12H) ppm. ESI-MS m/z calc. 522.24, found 545.3 (M+Na).

### Step 2: Ethyl (2R,8S)-8-((tert-butyldiphenylsilyl)oxy)-2-hydroxy-2-(trifluoromethyl)nonanoate (20)

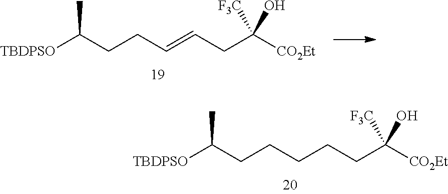

Ethyl (2R,8S,E)-8-((tert-butyldiphenylsilyl)oxy)-2-hydroxy-2-(trifluoromethyl)non-4-enoate (10.06 g, 19.247 mmol, 1 equiv.) was dissolved in EtOAc (201.2 mL, 0.096 M, 20 Vols), cycled 3 times between vacuum and N₂, treated with 10% Pd/C 50% water wet Pd/C (2.012 g, Evonik NOBLYST®P1173, Aldrich cat #330108), cycled 3 times between vacuum and hydrogen. The mixture was stirred at ambient under hydrogen balloon. The reaction was complete after 3 h by LC analysis. The mixture was cycled 3-times with between vacuum and nitrogen, filtered over celite, washing the celite pad with EtOAc. The filtrate was evaporated, swap with heptane and dried under house vacuum at ambient overnight to give the crude ethyl (2R,8S)-8-((tert-butyldiphenylsilyl)oxy)-2-hydroxy-2-(trifluoromethyl)nonanoate as a colorless oil (10.03 g, 99% yield). ¹H NMR (400 MHz, DMSO-d₆) δ 7.61 (dt, J=7.7, 1.5 Hz, 4H), 7.51-7.36 (m, 6H), 6.71 (s, 1H), 4.22 (q, J=7.1 Hz, 2H), 3.81 (h, J=6.0 Hz, 1H), 1.85 (td, J=12.7, 4.6 Hz, 1H), 1.64 (ddd, J=13.5, 11.8, 4.4 Hz, 1H), 1.47-1.28 (m, 3H), 1.28-1.09 (m, 7H), 1.00 (d, J=3.1 Hz, 13H) ppm. ESI-MS m/z calc. 524.26, found 525.34 (M+1)⁺.

### Step 3: Ethyl (2R,8S)-2-(benzyloxy)-8-((tert-butyldiphenylsilyl)oxy)-2-(trifluoromethyl)nonanoate (21)

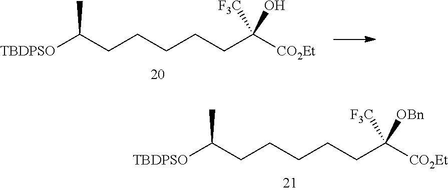

In a 250 mL RBF, ethyl (2R,8S)-8-((tert-butyldiphenylsilyl)oxy)-2-hydroxy-2-(trifluoromethyl)nonanoate (10.5 g, 20.012 mmol, 1 equiv.) in DMF (63 mL, 0.318 M, 6 Vols) was added benzyl bromide (4.191 g, 2.914 mL, 1.438 g/mL, 24.014 mmol, 1.2 equiv.). The clear mixture was stirred at ambient temperature and cesium carbonate (13.04 g, 40.023 mmol, 2 equiv.) was added as solid followed by TBAI (1.05 g, 2.843 mmol, 0.142 equiv.). Stirred at ambient until complete reaction by LC analysis (>5 h). The reaction mixture was diluted with EtOAc (100 mL) and water (50 mL). The mixture was stirred for 10 min and loaded into a separation funnel. The organic layer (light yellow) was isolated, was washed with brine/water (1:1, 20 mL×2, 10 mL×1), dried over Na₂SO₄, and concentrated via rotary evaporation. The residue was purified by column chromatography (220 g HP Silica) eluted with EtOAc/hexanes (0% for 3 min, 0-3% for 10 min, and then 3% for 11 min) over 24 min. The clean fractions were collected and concentrated, diluted with heptane, and dried under house vacuum at ambient overnight affording 11.96 g (97%) of ethyl (2R,8S)-2-(benzyloxy)-8-((tert-butyldiphenylsilyl)oxy)-2-(trifluoromethyl)nonanoate as a colorless oil. ¹H NMR (400 MHz, DMSO) δ 7.64-7.56 (m, 4H), 7.49-7.26 (m, 11H), 4.74-4.56 (m, 2H), 4.29 (q, J=7.1 Hz, 2H), 3.80 (h, J=5.9 Hz, 1H), 1.93 (dd, J=9.8, 5.5 Hz, 2H), 1.47-1.09 (m, 11H), 1.04-0.95 (m, 12H) ppm. ESI-MS m/z calc. 614.30, found 615.39 (M+1)⁺.

### Step 4: (2R,8S)-2-(Benzyloxy)-8-((tert-butyldiphenylsilyl)oxy)-2-(trifluoromethyl)nonanehydrazide (22)

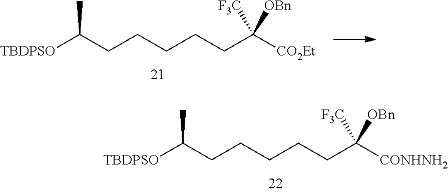

In a 40 mL of clear vial, ethyl (2R,8S)-2-(benzyloxy)-8-((tert-butyldiphenylsilyl)oxy)-2-(trifluoromethyl)nonanoate (3 g, 4.879 mmol, 1 equiv.) in THF (6 mL, 0.813 M, 2 Vols) was added 3,4,6,7,8,9-hexahydro-2H-pyrimido[1,2-a]pyrimidine (TBD, 207.923 mg, 1.464 mmol, 0.3 equiv.). Hydrazine monohydrate (572.505 mg, 64 w/w %, 7.319 mmol, 1.5 equiv.) was added drop wise to the mixture. The mixture was stirred at ambient temperature Stirred overnight. The reaction mixture was diluted with EtOAc (30 mL) and water (20 mL), stirred and phase split. The organic layer was washed with sat. NaHCO₃ (20 mL), brine/water (1:1, 20 mL), UPLC checked all the layers. The organic layer was dried over Na₂SO₄, concentrated on rotary evaporator. The residue was purified by column chromatography (40 g silica gel) eluting with EtOAc/hexane (0-40% for 14 min), clean fractions were collected and concentrated, swapped solvent with heptane, dried under house vacuum at ambient to give 2.6 g (2R,8S)-2-(benzyloxy)-8-((tert-butyldiphenylsilyl)oxy)-2-(trifluoromethyl)nonanehydrazide as colorless oil (88%). Preferred HPLC eluent for this reaction: 50-99% MeCN over 5 min with 10 mM NH₄HCO₂ in water (TFA buffer is acceptable for the first two steps). ¹H NMR (400 MHz, DMSO) δ 9.26 (s, 1H), 7.63-7.56 (m, 4H), 7.50-7.25 (m, 11H), 4.71-4.58 (m, 2H), 4.44 (d, J=4.3 Hz, 2H), 3.79 (h, J=6.0 Hz, 1H), 2.01 (q, J=6.6 Hz, 2H), 1.44-1.08 (m, 8H, corrected for presence of residual heptane), 1.00 (d, J=7.8 Hz, 12H) ppm. ESI-MS m/z calc. 600.30, found 632.30 (M+18)⁺.

### Intermediate 3: Preparation of 6-Bromo-3-(tert-butoxycarbonylamino)-5-(trifluoromethyl)pyridine-2-carboxylic acid (28)

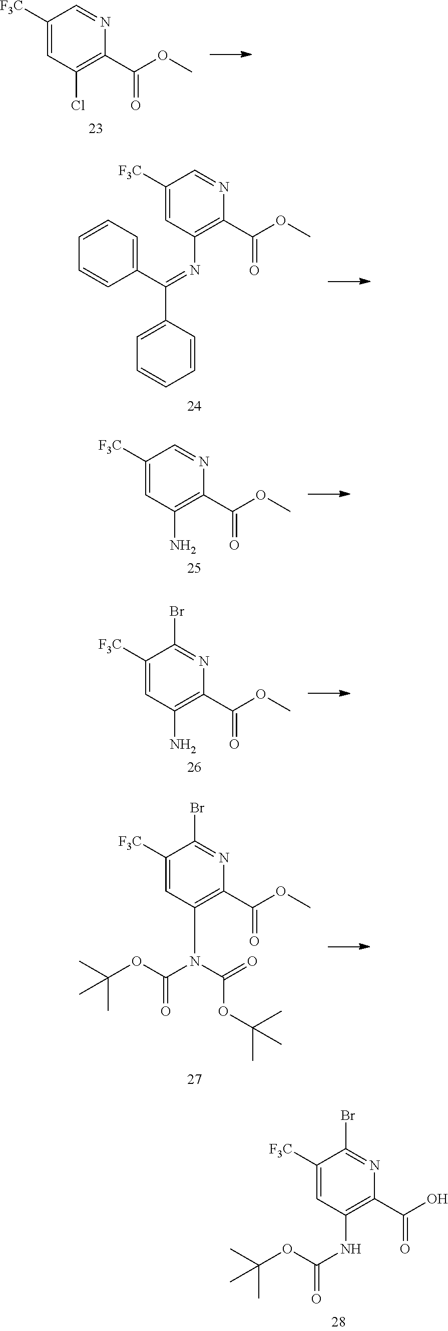 [SMILES: COC(=O)c1ncc(C(F)(F)F)cc1Cl ; COC(=O)c1ncc(C(F)(F)F)cc1N=C(c1ccccc1)c1ccccc1 ; COC(=O)c1ncc(C(F)(F)F)cc1N ; COC(=O)c1nc(Br)c(C(F)(F)F)cc1N ; COC(=O)c1nc(Br)c(C(F)(F)F)cc1N(C(=O)OC(C)(C)C)C(=O)OC(C)(C)C ; CC(C)(C)OC(=O)Nc1cc(C(F)(F)F)c(Br)nc1C(=O)O]

### Step 1: Methyl 3-(benzhydrylideneamino)-5-(trifluoromethyl)pyridine-2-carboxylate (24)

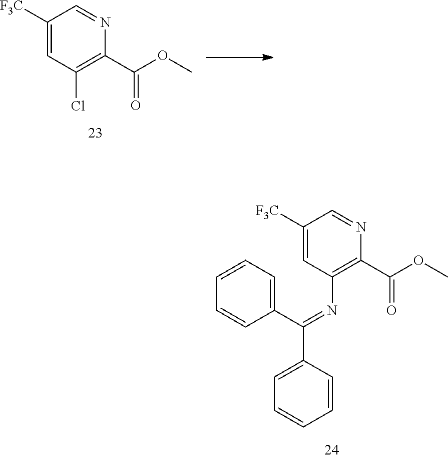 [SMILES: COC(=O)c1ncc(C(F)(F)F)cc1Cl ; COC(=O)c1ncc(C(F)(F)F)cc1N=C(c1ccccc1)c1ccccc1]

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 h. The reaction was cooled to room temperature and filtered on a pad of celite. The cake was washed with EtOAc and solvents were evaporated under reduced pressure to give methyl 3-(benzhydrylideneamino)-5-(trifluoromethyl)pyridine-2-carboxylate (90 g, 84%) as a yellow solid. ESI-MS m/z calc. 384.10855, found 385.1 (M+1)⁺; Retention time: 2.24 minutes. LCMS Method: Kinetex C₁₈ 4.6×50 mm 2.6 μM, 2.0 mL/min, 95% H₂O (0.1% formic acid)⁺5% acetonitrile (0.1% formic acid) to 95% acetonitrile (0.1% formic acid) gradient (2.0 min) then held at 95% acetonitrile (0.1% formic acid) for 1.0 min.

### Step 2: Methyl 3-amino-5-(trifluoromethyl)pyridine-2-carboxylate (25)

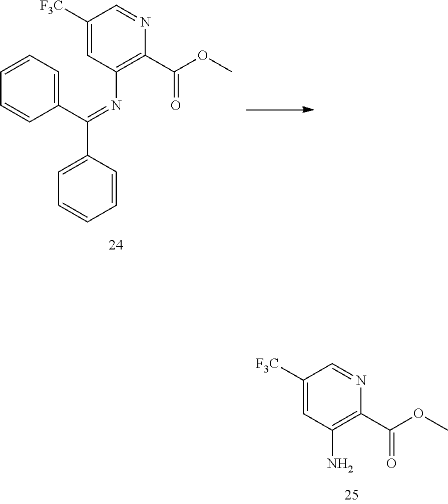 [SMILES: COC(=O)c1ncc(C(F)(F)F)cc1N=C(c1ccccc1)c1ccccc1 ; COC(=O)c1ncc(C(F)(F)F)cc1N]

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hour then the solvent was removed under reduced pressure. The residue was diluted with ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and then concentrated under reduced pressure. The residue was triturated with heptanes (2×50 mL) and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as a yellow solid. ¹H NMR (300 MHz, CDCl₃) δ 8.24 (s, 1H), 7.28 (s, 1H), 5.98 (br. s, 2H), 4.00 (s, 3H) ppm. ¹⁹F NMR (282 MHz, CDCl₃) δ −63.23 (s, 3F) ppm. ESI-MS m/z calc. 220.046, found 221.1 (M+1)⁺; Retention time: 1.62 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate (26)

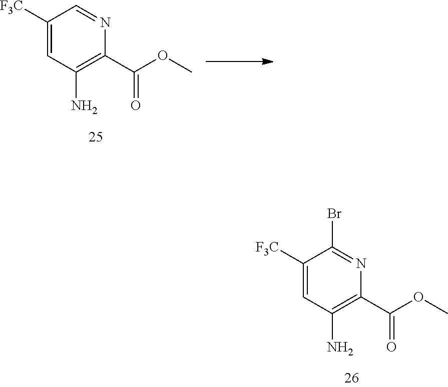 [SMILES: COC(=O)c1ncc(C(F)(F)F)cc1N ; COC(=O)c1nc(Br)c(C(F)(F)F)cc1N]

To a solution of methyl 3-amino-5-(trifluoromethyl)pyridine-2-carboxylate (18.75 g, 80.91 mmol) in acetonitrile (300 mL) at 0° C., N-bromosuccinimide (18.7 g, 105.3 mmol) was added portion wise. The mixture was stirred overnight at 25° C. Ethyl acetate (1000 mL) was added. The organic layer was washed with 10% aqueous sodium thiosulfate solution (3×200 mL), and the combined aqueous phases were extracted with ethyl acetate (2×200 mL). The combined organic extracts were then washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s, 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈ 4.6×50 mm 2.6 M. Temp: 45° C., Flow: 2.0 mL/min, Run Time: 6 min. Mobile Phase: Initial 95% H₂O (0.1% formic acid) and 5% acetonitrile (0.1% formic acid) linear gradient to 95% acetonitrile (0.1% formic acid) for 4.0 min then held at 95% acetonitrile (0.1% formic acid) for 2.0 min.

### Step 4: Methyl 3-[bis(tert-butoxycarbonyl)amino]-6-bromo-5-(trifluoromethyl)pyridine-2-carboxylate (27)

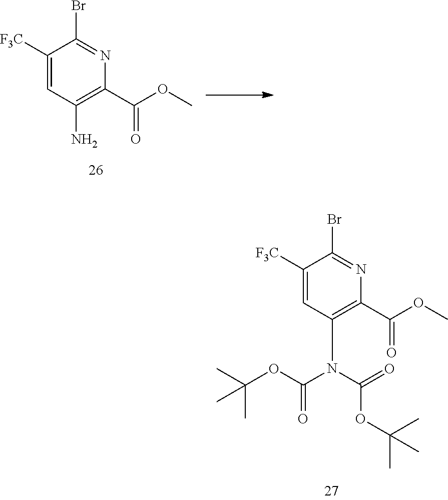 [SMILES: COC(=O)c1nc(Br)c(C(F)(F)F)cc1N ; COC(=O)c1nc(Br)c(C(F)(F)F)cc1N(C(=O)OC(C)(C)C)C(=O)OC(C)(C)C]

A mixture of methyl 3-amino-6-bromo-5-(trifluoromethyl)pyridine-2-carboxylate (5 g, 15.549 mmol), (Boc)₂O (11 g, 11.579 mL, 50.402 mmol), DMAP (310 mg, 2.5375 mmol) and CH₂Cl₂ (150 mL) was stirred at room temperature overnight. The reaction mixture was concentrated under reduced pressure and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ −63.9 (s, 3F) ppm. ESI-MS m/z calc. 498.06134, Retention time: 2.34 minutes. LCMS Method: Kinetex C18 4.6×50 mm 2.6 M. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Step 5: 6-Bromo-3-(tert-butoxycarbonylamino)-5-(trifluoromethyl)pyridine-2-carboxylic acid (28)

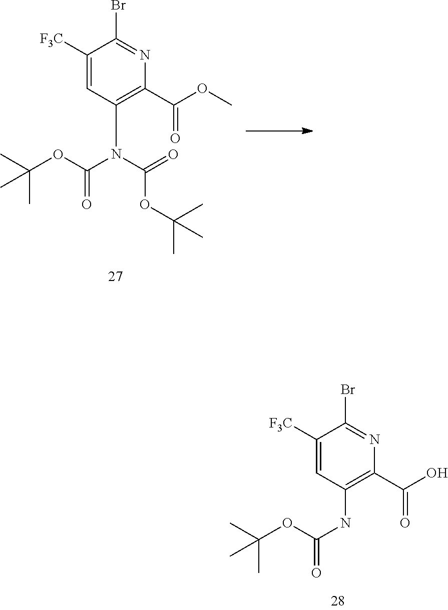 [SMILES: COC(=O)c1nc(Br)c(C(F)(F)F)cc1N(C(=O)OC(C)(C)C)C(=O)OC(C)(C)C ; CC(C)(C)OC(=O)Nc1cc(C(F)(F)F)c(Br)nc1C(=O)O]

To a mixture of methyl 3-[bis(tert-butoxycarbonyl)amino]-6-bromo-5-(trifluoromethyl)pyridine-2-carboxylate (247 g, 494.7 mmol) in THF (1.0 L) was added a solution of LiOH (47.2 g, 1.971 mol) in water (500 mL). The mixture was stirred at ambient temperature for 18 h affording a yellow slurry. The mixture was cooled with an ice-bath and slowly acidified with HCl (1000 mL of 2 M, 2.000 mol) keeping the reaction temperature <15° C. The mixture was diluted with heptane (1.5 L), mixed and the organic phase separated. The aqueous phase was extracted with heptane (500 mL). The combined organic phases were washed with brine, dried over MgSO₄, filtered, and concentrated in vacuo. The crude oil was dissolved in heptane (600 mL), seeded and stirred at ambient temperature for 18 h affording a thick slurry. The slurry was diluted with cold heptane (500 mL) and the precipitate collected using a medium frit. The filter cake was washed with cold heptane and air dried for 1 h, then in vacuo at 45° C. for 48 h to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 384.9 (M+1)⁺; Retention time: 2.55 minutes. LCMS Method Detail: Final purity was determined by reversed phase UPLC using an Acquity UPLC BEH C18 column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 4: Preparation of (2R,8R)-2-(Benzyloxy)-8-((tert-butyldiphenylsilyl)oxy)-2-(trifluoromethyl)nonanehydrazide (29)

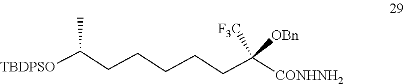

Compound 29 can be prepared using (S)-tert-butyl(hept-6-en-2-yloxy)diphenylsilane in a manner analogous to that disclosed for Intermediate 2. The synthesis of compound 18 is disclosed in, for example, Gaddam, et al. Org. Biomol. Chem., 2019, 17, 5601-5614.

### Intermediate 5: Preparation of (2R)-2-Benzyloxy-2-(trifluoromethyl)hex-5-enehydrazide (34)

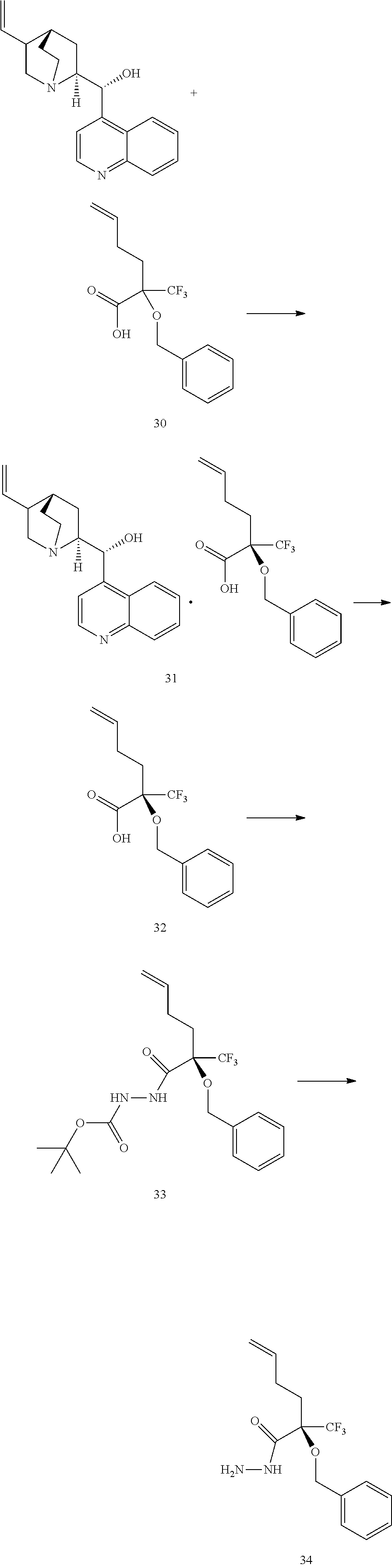

### Step 1: (2R)-2-Benzyloxy-2-(trifluoromethyl)hex-5-enoic acid; (R)-4-Quinolyl-[(2S,4S)-5-vinylquinuclidin-2-yl]methanol (31)

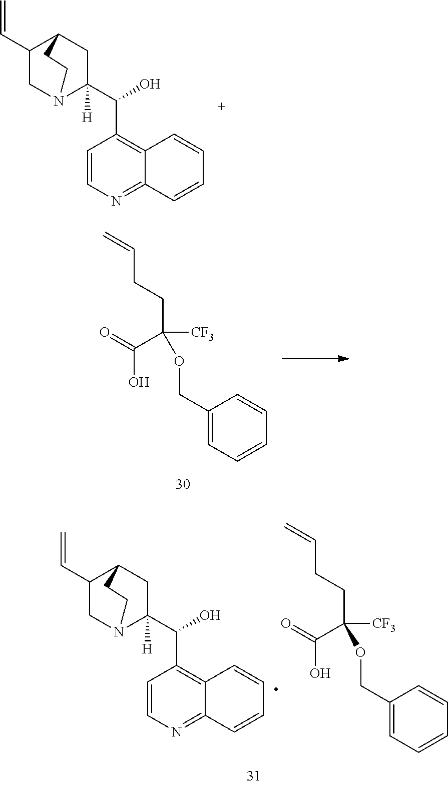

To a N₂ purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with a minimal amount of IPAC. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 h, then stirred overnight at 20° C. The material was then filtered and cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under a nitrogen atmosphere, heated to 80° C. and held at temperature at least 10 minutes, then ramped to 20° C. over 4-6 hours, and then stirred overnight. The mixture was filtered, cake washed with isopropyl acetate (6.2 L, 2 vol), pulled dry, scooped out and dried in vacuo (50° C., vacuum) to afford 2.25 kg of solid salt. The solid (2.25 kg) and isopropyl acetate (45 L, 20 vol based on salt solid wt) was slurried and added to a reactor, stirred under nitrogen atmosphere and heated to 80° C., held at temperature at least 10 minutes, then ramped to 20° C. over 4-6 hours, then stirred overnight. The mixture was filtered, cake washed with isopropyl acetate (4.5 L, 2 vol), air dried in the filter, and dried in vacuo at 50° C. to afford (2R)-2-benzyloxy-2-(trifluoromethyl)hex-5-enoic acid; (R)-4-quinolyl-[(2S,4S)-5-vinylquinuclidin-2-yl]methanol (1.886 kg, >98.0% ee) as off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.1% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic acid (32)

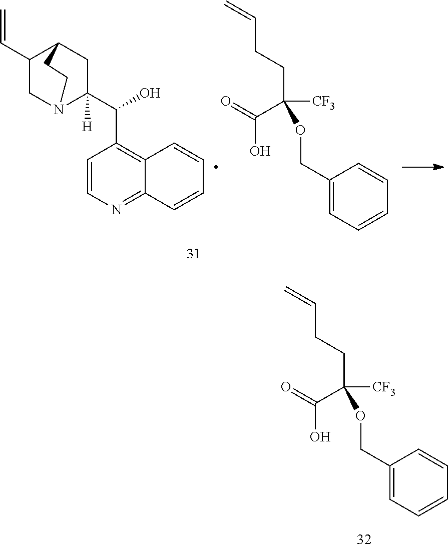 [SMILES: C=CC1CN2CC[C@H]1C[C@H]2[C@H](O)c1ccnc2ccccc12 ; C=CCC[C@@](OCc1ccccc1)(C(=O)O)C(F)(F)F ; C=CCC[C@@](OCc1ccccc1)(C(=O)O)C(F)(F)F]

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ −71.63 (br s, 3F) ppm. ESI-MS m/z calc. 288.0973, found 287.0 (M−1)⁻; Retention time: 2.15 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: tert-Butyl N-[[(2R)-2-benzyloxy-2-(trifluoromethyl)hex-5-enoyl]amino]carbamate (33)

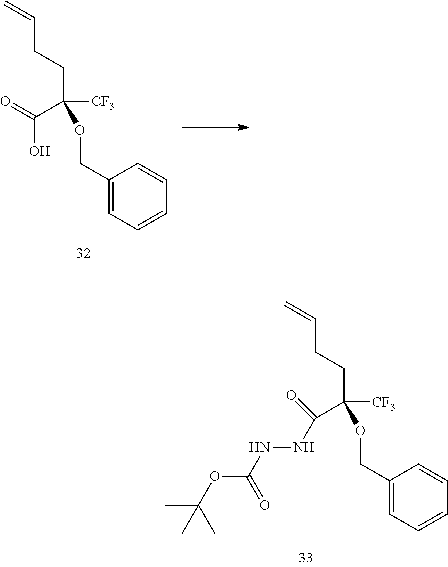 [SMILES: C=CCC[C@@](OCc1ccccc1)(C(=O)O)C(F)(F)F ; C=CCC[C@@](OCc1ccccc1)(C(=O)NNC(=O)OC(C)(C)C)C(F)(F)F]

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 min. To the mixture was added tert-butyl N-aminocarbamate (200 g, 1.513 mol) (slight exotherm upon addition) and the mixture was stirred at ambient temperature for 16 h. The reaction was poured into ice water (5 L). The resultant precipitate was collected by filtration and washed with water. The solid was dissolved in EtOAc (2 L) and washed with brine. The organic phase was dried over magnesium sulfate, filtered, and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purified by silica gel chromatography (0-40% EtOAc/hexanes) to afford tert-butyl N-[[(2R)-2-benzyloxy-2-(trifluoromethyl)hex-5-enoyl]amino]carbamate (464 g, 91%, combined with product from crystallization). ESI-MS m/z calc. 402.17664, found 303.0 (M+1-Boc)⁺; Retention time: 2.68 minutes. Final purity was determined by reversed phase UPLC using an Acquity UPLC BEH C₁₈ column (50×2.1 mm, 1.7 μm particle) made by Waters (pn: 186002350) and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 4: (2R)-2-Benzyloxy-2-(trifluoromethyl)hex-5-enehydrazide (34)

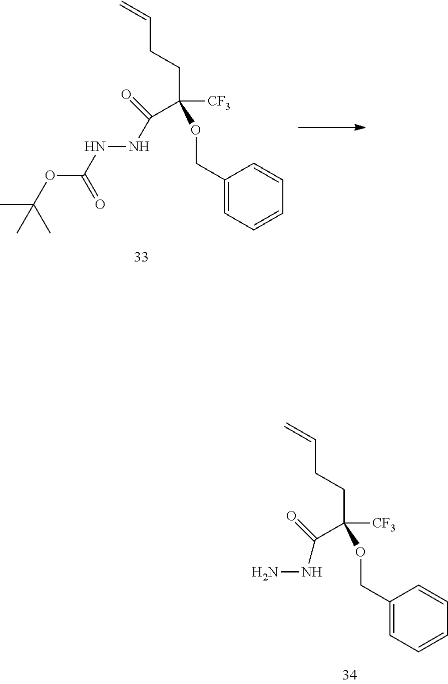 [SMILES: C=CCC[C@@](OCc1ccccc1)(C(=O)NNC(=O)OC(C)(C)C)C(F)(F)F ; C=CCC[C@@](OCc1ccccc1)(C(=O)NN)C(F)(F)F]

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 h. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with 1 L of brine and the combined aqueous phases were extracted with isopropyl acetate (1 L). The combined organic phases were dried over magnesium sulfate, filtered and concentrated in vacuo to afford a dark yellow oil of (2R)-2-benzyloxy-2-(trifluoromethyl)hex-5-enehydrazide (358 g, quantitative yield). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 6: Preparation of 3-((tert-Butoxycarbonyl)amino)-6-(methylthio)-5-(trifluoromethyl)picolinic acid (39)

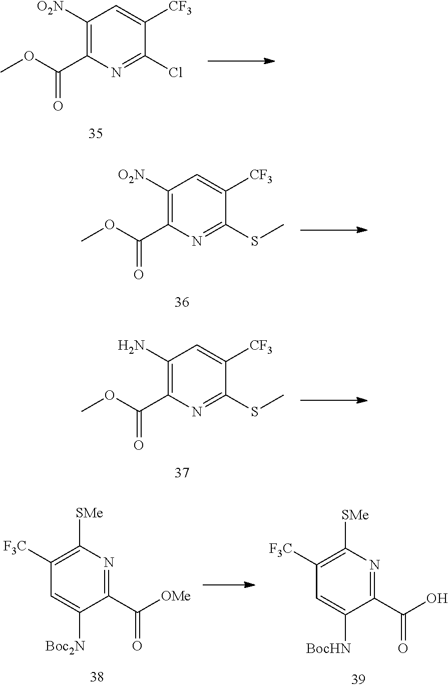

### Step 1: Preparation of Methyl 6-(methylthio)-3-nitro-5-(trifluoromethyl)picolinate (36)

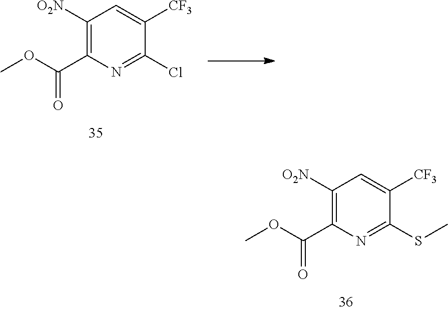

To a solution of NaSMe (29.56 g, 421.68 mmol, 26.87 mL, 1.2 eq) in THF (750 mL) and H₂O (250 mL) was added compound 35 (100 g, 351.40 mmol, 1 eq) portion wise at 0° C. (the mixture turned from colorless suspension to brown clear solution). The mixture was stirred at 20° C. for 2 hours. The mixture was extracted with EtOAc (200 mL). The organic layer was dried with sodium sulfate, filtered, and concentrated in vacuum. The residue was purified by flash MPLC (SiO₂, petroleum ether/ethyl acetate from petroleum ether to 20/1) to give a crude product which was further purified by trituration with petroleum ether/ethyl acetate=100/1 (300 mL), filtered and the cake was collected and dried under vacuum to give methyl 6-(methylthio)-3-nitro-5-(trifluoromethyl)picolinate (65 g, 219.43 mmol, 62.44% yield, 100% purity) as a white solid. LCMS of methyl 6-(methylthio)-3-nitro-5-(trifluoromethyl)picolinate: retention time=0.872 min, m z 297.0 [M+H]⁺. ¹H NMR of methyl 6-(methylthio)-3-nitro-5-(trifluoromethyl)picolinate (400 MHz, CDCl₃): δ 8.56 (s, 1H), 4.06 (s, 3H), 2.71 (s, 3H) ppm.

### Step 2: Preparation of Methyl 3-amino-6-(methylthio)-5-(trifluoromethyl)picolinate (37)

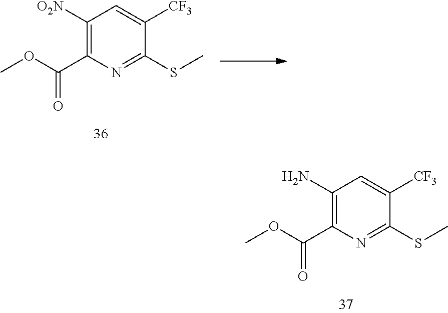

To a solution of methyl 6-(methylthio)-3-nitro-5-(trifluoromethyl)picolinate (45 g, 151.91 mmol, 1 eq) in THF (300 mL) and H₂O (150 mL) was added Fe (84.84 g, 1.52 mol, 10 eq) and NH₄Cl (81.26 g, 1.52 mol, 10 eq). The mixture was heated to 70° C. and stirred for 12 hours. LCMS showed the starting material was consumed and desired mass was detected. The mixture was cooled to room temperature, then filtered and the cake was washed with EtOAc (500 mL). Organic layer was collected, washed brine (100 mL), dried over Na₂SO₄, filtered, and concentrated in vacuum. The residue was purified by column chromatography (SiO₂, petroleum ether/ethyl acetate from 20/1 to 10/1) to give a crude product (40 g). The crude product was further purified by trituraton with petroleum ether/ethyl acetate (10/1, 100 mL), filtered and the cake was collected to give methyl 3-amino-6-(methylthio)-5-(trifluoromethyl)picolinate (30 g, 111.55 mmol, 73.43% yield, 99% purity) as a yellow solid. LCMS of methyl 3-amino-6-(methylthio)-5-(trifluoromethyl)picolinate: retention time=0.829 min, m z 267.1 [M+H]⁺. HPLC of methyl 3-amino-6-(methylthio)-5-(trifluoromethyl)picolinate: retention time=2.233 min, purity: 99.5%. ¹H NMR (400 MHz, DMSO-d₆): δ 7.67 (s, 1H), 6.82 (s, 2H), 3.85 (s, 3H), 2.52 (s, 3H) ppm.

### Step 3: Preparation of Methyl 3-(bis(tert-butoxycarbonyl)amino)-6-(methylthio)-5-(trifluoromethyl)picolinate (38)

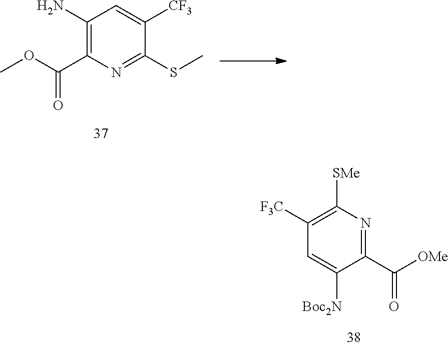

Methyl 3-amino-6-(methylthio)-5-(trifluoromethyl)picolinate (8 g, 30 mmol, 1 eq) in 60 mL of 2-MeTHF. DMAP (0.73 g, 6 mmol, 0.2 eq) was added followed by dropwise addition of Boc₂O (19.7 g, 90.1 mmol, 3 eq) in 20 mL 2-MeTHF via addition funnel. The temperature rose 6° C. during addition with visible gas evolution. After addition, reaction monitored for completion by LC analysis. 40 mL of water added. After stirring, clean phase split. Organic layer dried (Na₂SO₄), filtered, and concentrated to a solid. 80 mL heptane added and concentrated. The crude product was triturated with 50 mL of heptane. Slurry stirred at ambient temperature After stirring overnight, the solid was collected by filtration and washed with heptane (2×10 mL). The solid was dried in a vacuum oven at 50° C. with nitrogen bleed to afford 11.87 g (84% yield) of methyl 3-(bis(tert-butoxycarbonyl)amino)-6-(methylthio)-5-(trifluoromethyl)picolinate. ¹H NMR (400 MHz, DMSO-d₆) δ 8.41 (s, 1H), 3.88 (s, 3H), 2.64 (s, 3H), 1.35 (s, 18H) ppm. ESI-MS m/z calc. 466.14, found 467.0 (M+1)⁺.

### Step 4: Preparation of 3-((tert-Butoxycarbonyl)amino)-6-(methylthio)-5-(trifluoromethyl)picolinic acid (39)

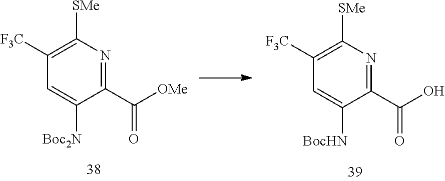

Methyl 3-(bis(tert-butoxycarbonyl)amino)-6-(methylthio)-5-(trifluoromethyl)picolinate (11 g, 23.6 mmol, 1 eq) was diluted with 77 mL of THF. LiOH H₂O (9.9 g, 236 mmol, 10 eq) in 77 mL of water added dropwise via addition funnel and the 2-phase mixture stirred vigorously at ambient temperature. The reaction was monitored for completion by LC analysis of an aliquot from the organic layer. After complete reaction, as determined by LC, 6 M HCl added dropwise. A total of 45 mL (270 mmol) was added to adjust the pH to <3 (as determined using pH paper). 2-MeTHF (70 mL) added. After stirring, the organic layer was isolated, dried over sodium sulfate, filtered, and concentrated to a solid. 70 mLThe solid was diluted with heptane and concentrated. The resulting solid was triturated with 50 mL of heptane. The solid was collected by filtration and washed with heptane (2×7 mL) and further dried in vacuum oven at 50° C. with nitrogen bleed to afford 7.01 g (84% yield) of 3-((tert-butoxycarbonyl)amino)-6-(methylthio)-5-(trifluoromethyl)picolinic acid. ¹H NMR (400 MHz, DMSO-d₆) δ 13.91 (s, 1H), 10.18 (s, 1H), 8.85 (s, 1H), 2.60 (s, 3H), 1.49 (s, 9H) ppm. ESI-MS m/z calc. 352.07, found 296.9 (M+1-C₄H₈ from Boc group)⁺.

### Intermediate 7: Preparation of [(3S)-3-Hydroxybutyl]-triphenyl-phosphonium iodide (41)

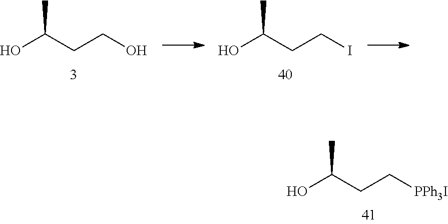

### Step 1: (2S)-4-iodobutan-2-ol (40)

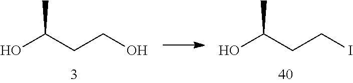

Triphenylphosphine (4.32 g, 3.8163 mL, 16.471 mmol) was dissolved in DCM (28 mL) at RT. I₂ (4.22 g, 0.8560 mL, 16.627 mmol) was added, followed by addition of polymer-bound DMAP (Sigma Aldrich Catalog No. 39410) 3 mmol/g (2 g, 26% w/w, 4.2564 mmol). The brownish mixture was stirred at RT for several minutes. (3S)-butane-1,3-diol (1 g, 11.096 mmol) in DCM (6 mL) was added quickly via pipette. Mixture was stirred for 1 hr. Na₂S₂O₃ (20 ml, sat. aq.) was added. Layers were separated. The DCM layer was dried over anh. MgSO₄, filtered and concentrated. The residue was purified by silica gel chromatography (40 g column), using 5-50% EtOAc in hexanes, to afford the desired product (2S)-4-iodobutan-2-ol (1.5 g, 64%) as pale yellow oil. ¹H NMR (500 MHz, Chloroform-d) δ 4.02-3.82 (m, 1H), 3.41-3.15 (m, 2H), 2.07-1.84 (m, 2H), 1.46 (s, 1H), 1.33-1.15 (m, 3H). ¹H NMR (500 MHz, Chloroform-d) δ 4.02-3.82 (m, 1H), 3.41-3.15 (m, 2H), 2.07-1.84 (m, 2H), 1.46 (s, 1H), 1.33-1.15 (m, 3H) ppm.

### Step 2: [(3S)-3-Hydroxybutyl]triphenylphosphonium iodide (41)

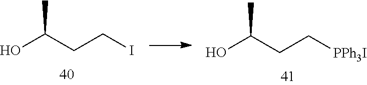

(2S)-4-Iodobutan-2-ol (1.14 g, 5.4145 mmol) was dissolved in CH₃CN (15 mL) at RT. Triphenylphosphine (1.42 g, 5.4140 mmol) was added. The mixture was stirred in an 83° C. oil bath under a N₂ balloon for 24 hours. The cloudy mixture was cooled to RT. The mixture was decanted and the precipitate was washed with more CH₃CN (10 ml×2) and decanted each time. The solid was concentrated to afford the desired [(3S)-3-hydroxybutyl]-triphenyl-phosphonium iodide (2.2 g, 86%) as a white solid. ESI-MS m/z calc. 462.0609, found 335.4 (M−127)⁺; Retention time: 2.18 minutes. LCMS Method: Merck Millipore Chromolith SpeedROD C18 column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

### Example 24: Preparation of (6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol (Compound I)

### Step 1: Methyl (R)-3-(bis(tert-butoxycarbonyl)amino)-6-(pent-4-en-2-yloxy)-5-(trifluoromethyl)picolinate (43)

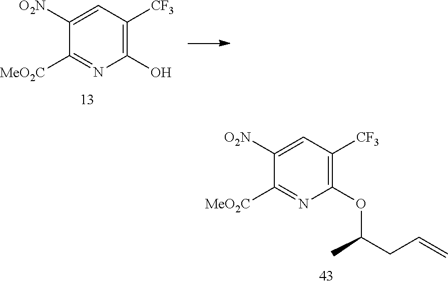 [SMILES: COC(=O)c1nc(O)c(C(F)(F)F)cc1[N+](=O)[O-] ; C=CC[C@@H](C)Oc1nc(C(=O)OC)c([N+](=O)[O-])cc1C(F)(F)F]

In a 50 L reactor with condenser was added methyl 6-hydroxy-3-nitro-5-(trifluoromethyl)pyridine-2-carboxylate (1300 g, 4884.794 mmol, 1 eq), 4-penten-2-ol (462.822 g, 5373.273 mmol, 1.1 eq), triphenyl phosphine (1537.453 g, 5861.753 mmol, 1.2 eq), and toluene (13000 mL, 0.376 M, 10 Vols). The jacket temp was set to 20° C. and stirred ˜8 min. The reaction temp was then set to 0° C. Started the addition of diisopropyl azodicarboxylate (1185.292 g, 1154.131 mL, 1.027 g/mL, 5861.753 mmol, 1.2 eq) via a 500 mL addition funnel. After addition was complete, the reaction temperature was increased to 20° C. over 30 min. Stirred until reaction complete <1% (AUC) of SM by LC analysis. An overnight sample for HPLC analysis showed there was no starting material present. Added magnesium chloride (1116.2 g, 11723.506 mmol, 2.4 eq) to the reaction mixture and diluted it with heptane (13000 mL, 0.376 M, 10 Vols). After the additions were complete, the reaction temperature was set to 60° C. The reaction mixture was set to stir ˜2 h. After 2 h, the reaction temperature was cooled down to 20° C. and left mixing for approx. 10 min at room temp. Filtered out solids using a 5 L filter. The resulting filtrate was concentrated via rotary evaporation. Left the reaction mixture in heptane (˜4 L) and mixed overnight on the rotary evaporator. Packed 5 L column with both silica and Celite. Ran the reaction mixture through to get rid of any solids and rinsed with 3 L of heptane. Concentrated the filtrate and obtained 1675 g (94%) of methyl (R)-3-(bis(tert-butoxycarbonyl)amino)-6-(pent-4-en-2-yloxy)-5-(trifluoromethyl)picolinate. ¹H NMR (400 MHz, DMSO) δ 8.77 (s, 1H), 5.82 (ddt, J=17.2, 10.2, 7.0 Hz, 1H), 5.50 (q, J=6.1 Hz, 1H), 5.19-5.03 (m, 2H), 3.99 (s, 3H), 2.51 (tdd, J=7.5, 3.9, 2.5 Hz, 2H), 1.38 (d, J=6.3 Hz, 3H) ppm. ESI-MS m/z calc. 334.08, found 267 (M+1)⁺.

### Step 2: Methyl (R)-3-amino-6-(pent-4-en-2-yloxy)-5-(trifluoromethyl)picolinate (44)

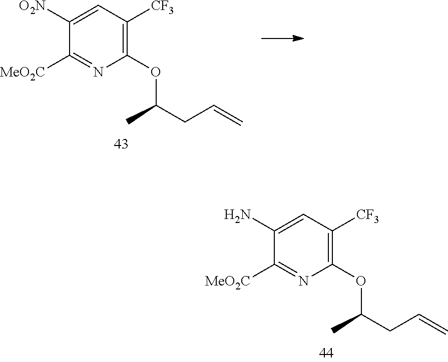 [SMILES: C=CC[C@@H](C)Oc1nc(C(=O)OC)c([N+](=O)[O-])cc1C(F)(F)F ; C=CC[C@@H](C)Oc1nc(C(=O)OC)c(N)cc1C(F)(F)F]

Methyl (R)-3-nitro-6-[(2R)-pent-4-en-2-yloxy]-5-(trifluoromethyl)pyridine-2-carboxylate (5 g, 14.959 mmol, 1 eq) in tetrahydrofuran (25 mL, 0.598 M, 5 Vols) and EtOH (25 mL, 0.598 M, 5 Vols) stirred at ambient temperature Sodium dithionite (tech, 85%, 11.72 g (10 g actual), 57.3 mmol, 3.83 eq) in 75 mL of water added dropwise via addition funnel. Addition over 5 min, temp rose from 18 to 25.8° C. (maintain temp <30° C.). Stirred until reaction complete <1% (AUC) of SM by LC analysis. Added 50 mL of 2-MeTHF. After stirring, clean phase split. Organic layer washed with 50 mL 4:1 v/v water/brine. After stirring, clean phase split. 50 mL of 1 M HCl added to organic phase. After stirring for 30 min, clean phase split. Organic layer was washed with 40 mL of 10% w/v aq KHCO₃. After stirring, clean phase split. Organic layer dried (Na₂SO₄) and filtered. Removing solvent on rotary evaporator, swap solvent with heptane, dried under vacuum oven at 40-50° C. with N₂ bleed overnight to afford 4.32 g (95%) of methyl (R)-3-amino-6-(pent-4-en-2-yloxy)-5-(trifluoromethyl)picolinate. ¹H NMR (400 MHz, DMSO) δ 7.70 (d, J=0.7 Hz, 1H), 6.57 (s, 2H), 5.81 (ddt, J=17.2, 10.1, 7.0 Hz, 1H), 5.23-5.00 (m, 3H), 3.83 (s, 3H), 2.39 (tq, J=7.0, 1.2 Hz, 2H), 1.25 (d, J=6.2 Hz, 3H) ppm. ESI-MS m/z calc. 304.10, found 305.13 (M+1)⁺.

### Step 2: Methyl (R)-3-amino-6-(pent-4-en-2-yloxy)-5-(trifluoromethyl)picolinate (Alternative Conditions)

To the 50 L reactor (internal temperature of 20° C.) was added acetic acid (6696 mL, 0.686 M, 4 Vols) and H₂O (13392 mL, 0.343 M, 8 Vols). The reactor was degassed three times before adding iron powder, (−325 mesh, 97%; 2050.864 g, 36724.22 mmol, 8 eq) and additional acetic acid (83.7 mL, 54.845 M, 0.05 Vols). Methyl (R)-3-nitro-6-(pent-4-en-2-yloxy)-5-(trifluoromethyl)pyridine-2-carboxylate (1674 g, 4590.527 mmol, 1 eq) in EtOAc (6696 mL, 0.686 M, 4 Vols) via an additional funnel over ˜50 min. Stirred until near complete consumption of methyl (R)-3-nitro-6-(pent-4-en-2-yloxy)-5-(trifluoromethyl)pyridine-2-carboxylate was observed by LC. Added ˜1.2 kg of Celite into the reaction mixture and stirred the resulting mixture overnight. Filtered mixture through a pad of silica and Celite, and washed the silica and Celite with ethyl acetate (˜6 L). Allowed the mixture to stand and isolated the organic phase. Washed organic phase with water (5 Vol) and saturated aqueous sodium bicarbonate (3×, 4 Vols. Each wash). The organic phase was then dried over sodium sulfate, filtered, and concentrated in vacuo to afford the title compound (1448 g (91%)). This material was used without further purification.

### Step 3: Methyl (R)-3-(bis(tert-butoxycarbonyl)amino)-6-(pent-4-en-2-yloxy)-5-(trifluoromethyl)picolinate (45)

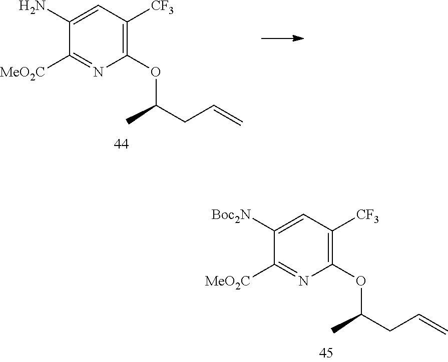

Started the 50 L reactor with stirrer at ˜100 rpm. Jacket temperature was adjusted to 20° C. 2-MeTHF (14470 mL, 0.329 M, 8 Vols), di-tert-butyl dicarbonate (2075.816 g, 9511.321 mmol, 2 eq), and 4-(dimethylamino)pyridine (58.099 g, 475.566 mmol, 0.1 equiv.) were added. Dissolved methyl (R)-3-amino-6-(pent-4-en-2-yloxy)-5-(trifluoromethyl)pyridine-2-carboxylate (1447 g, 4755.66 mmol, 1 eq) in 2-MeTHF (2894 mL, 1.643 M, 2 Vols) and added into the reactor via an addition funnel over ˜40 min. Stirred (20° C.) until nearly complete consumption of methyl (R)-3-amino-6-(pent-4-en-2-yloxy)-5-(trifluoromethyl)pyridine-2-carboxylate was observed by LC analysis. Added water (5 vol), stirred, and isolated the organic layer. The organic layer was dried over Na₂SO₄, filtered, and concentrated via rotary evaporation. The resulting residue was diluted with 4 L of heptane. The resulting mixture was then concentrated in vacuo and the title compound was dried for 2.5 hours under vacuum. The crude material was used without further purification.

### Step 4: (R)-3-((tert-Butoxycarbonyl)amino)-6-(pent-4-en-2-yloxy)-5-(trifluoromethyl)picolinic acid (46)

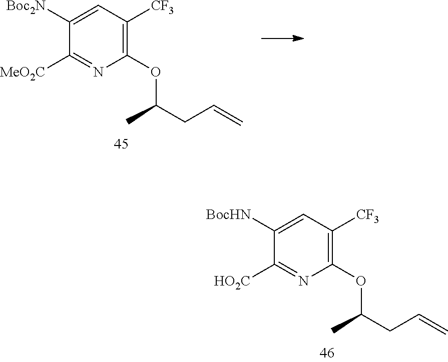

Added methyl (R)-3-[bis(tert-butoxycarbonyl)amino]-6-(pent-4-en-2-yloxy)-5-(trifluoromethyl)pyridine-2-carboxylate (2167 g, 4295.316 mmol, 1 eq) in THF (15169 mL, 0.283 M, 7 Vols) to a 50 L reactor. The mixture was stirred at 20° C., to which a solution of lithium hydroxide monohydrate (1802.47 g, 42953.164 mmol, 10 eq) in H₂O (8668 mL, 0.496 M, 4 Vols) was added (over ˜1.5 hrs). After addition was finished, the mixture was stirred until nearly complete consumption of methyl (R)-3-[bis(tert-butoxycarbonyl)amino]-6-(pent-4-en-2-yloxy)-5-(trifluoromethyl)pyridine-2-carboxylate was observed by LC analysis. The mixture was filted and the filtrated was concentrated in vacuo. Approximately 10 L of IPAc and 5 L of de-ionized water were added to an extractor. The crude mixture was diluted with 5 L of IPAc and added it to the extractor and the round bottom containing the crude mixture was washed with 3 L of IPAc, which was then added to the extractor. An additional 10 L of DI water and 7 L of IPAc were added to the extractor. The resulting mixture was mixed for approximately 15 minutes and then allowed to stand until phase separation was observed. The aqueous phase was isolated and was extracted with an additional 4 L of IPAc. The organic phases were combined and filtered using a 5 L filter. The organic phase was acidified with potassium bisulfate (10% aq w/v; 10 L). The resulting mixture was stirred for approximately 15 minutes and then allowed to stand until the phases separated. The organic phase was isolated, dried over sodium sulfate, filtered, and concentrated in vacuo. The resulting residue was diluted with acetonitrile (5 L) and concentrated to afford a dark oil (1694 g). This material was used without further purification.

### Step 4A (optional): (R)-3-((tert-Butoxycarbonyl)amino)-6-(pent-4-en-2-yloxy)-5-(trifluoromethyl)picolinate dicyclohexylammonium salt (46a)

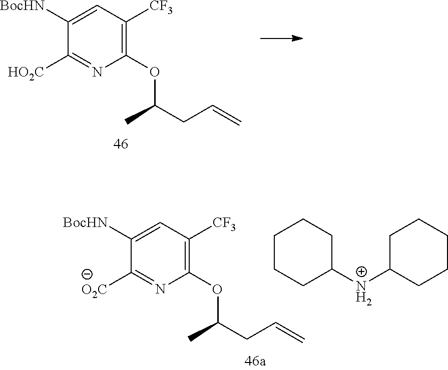

The 50 L reactor was set up, set Tj=20° C., and added 3-[(tert-butoxycarbonyl)amino]-6-(pent-4-en-2-yloxy)-5-(trifluoromethyl)pyridine-2-carboxylic acid (1693 g, 4337.033 mmol, 1 eq) dissolved in CH₃CN (6772 mL, 0.64 M, 4 Vols). The reaction temperature was set to 20° C. After reaction temperature was reached, the addition of dicyclohexylamine (707.743 g, 776.034 mL, 0.912 g/mL, 3903.33 mmol, 0.9 eq) was started using a 500 mL addition funnel. Addition was fast (˜10 min), with slight exothermic behavior. Reaction mixture stirred overnight. The solid was collected by filtration, rinsed with CH₃CN (0.8 L×2). The solid was dried under vacuum oven at 40° C. with N₂ bleed overnight to afford 1905 g of product as an off white solid (yield: 69% for 2 steps).

¹H NMR (400 MHz, DMSO) δ 12.54 (s, 1H), 8.76 (s, 1H), 5.89-5.74 (m, 1H), 5.37 (d, J=6.1 Hz, 1H), 5.15-5.00 (m, 2H), 3.04 (s, 1H), 2.47-2.35 (m, 2H), 1.97 (d, J=9.1 Hz, 4H), 1.73 (dd, J=8.9, 3.6 Hz, 4H), 1.60 (d, J=12.6 Hz, 3H), 1.46 (s, 9H), 1.37-1.16 (m, 13H), 1.09 (td, J=12.6, 3.6 Hz, 2H). ESI-MS m/z calc. 390.14, found 391.12 (M+1)⁺

### Step 5: tert-Butyl (2-(2-((R)-2-(benzyloxy)-2-(trifluoromethyl)hex-5-enoyl)hydrazine-1-carbonyl)-6-(((R)-pent-4-en-2-yl)oxy)-5-(trifluoromethyl)pyridin-3-yl)carbamate (47)

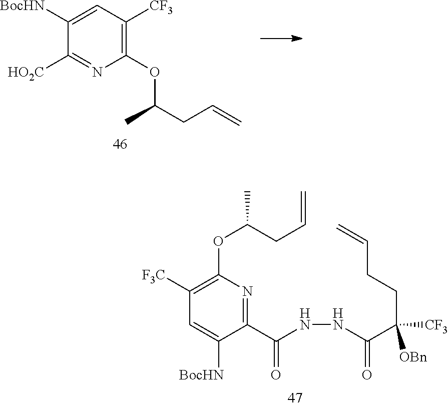 [SMILES: C=CC[C@@H](C)Oc1nc(C(=O)O)c(NC(=O)OC(C)(C)C)cc1C(F)(F)F ; C=CCC[C@@](OCc1ccccc1)(C(=O)NNC(=O)c1nc(O[C@H](C)CC=C)c(C(F)(F)F)cc1NC(=O)OC(C)(C)C)C(F)(F)F]

Added CDI (1756.034 g, 10829.775 mmol, 1.25 equiv.) and CH₃CN (10146 mL, 3 Vols) into a 100 L reactor (20° C. and stirring was set to −150 rpm). In a separate vessel, (R)-3-((tert-butoxycarbonyl)amino)-6-(pent-4-en-2-yloxy)-5-(trifluoromethyl)picolinic acid (3382 g, 8663.82 mmol, 1 equiv.) was diluted in CH₃CN (6764 mL, 2 Vols), which was added to the reactor over 30 min. After stirring for approximately 3 hours, (2R)-2-Benzyloxy-2-(trifluoromethyl)hex-5-enehydrazide (2880.951 g, 9530.202 mmol, 1.1 equiv.) was diluted with CH₃CN (6764 mL, 2 Vols) in a separate vessel. The resulting mixture was then added to the reactor over approximately 1 hour. After addition, the mixture was stirred for approximately 3 hours.

To a 100 L jacketed reactor was added water (23674 mL, 7 Vols) and, while mixing at 180 rpm, tert-butyl (2-(2-((R)-2-(benzyloxy)-2-(trifluoromethyl)hex-5-enoyl)hydrazine-1-carbonyl)-6-(((R)-pent-4-en-2-yl)oxy)-5-(trifluoromethyl)pyridin-3-yl)carbamate seed material (174 g) was added. Over the course of approximately 2 hours, the stirring mixture described in the previous paragraph was transferred to this aqueous mixture while stirring (˜193 rpm). The resulting mixture was left to stir overnight. The mixture was then filtered and the wet filter cake was washed with an acetonitrile/water mixture (1/1, 3382 mL, 1 Vols). The isolated material was dried under vacuum for approximately 1 hour at room temperature, and then further dried overnight under vacuum at 55° C. with nitrogen bleed to afford 5,336 g (91%) of the title compound as a white solid. ¹H NMR (400 MHz, DMSO) δ 10.73 (d, J=8.8 Hz, 2H), 10.34 (s, 1H), 9.03 (s, 1H), 7.54-7.47 (m, 2H), 7.43-7.34 (m, 2H), 7.37-7.28 (m, 1H), 5.94-5.75 (m, 2H), 5.15-4.98 (m, 5H), 4.91-4.79 (m, 2H), 2.48-2.29 (m, 4H), 2.20 (s, 2H), 1.48 (s, 9H), 1.26 (d, J=6.1 Hz, 3H) ppm. ESI-MS m/z calc. 674.25, found 675.66 (M+1)⁺.

### Alternative Step 5: tert-Butyl (2-(2-((R)-2-(benzyloxy)-2-(trifluoromethyl)hex-5-enoyl)hydrazine-1-carbonyl)-6-(((R)-pent-4-en-2-yl)oxy)-5-(trifluoromethyl)pyridin-3-yl)carbamate (47)

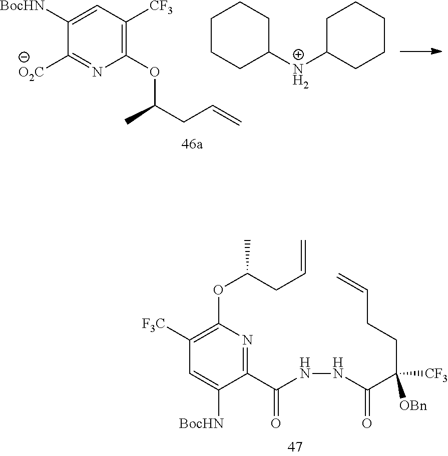

Compound 47 can be prepared from Compound 46a in a similar manner to Step 5, described above.

### Step 6: tert-Butyl (2-(5-((R)-2-(benzyloxy)-1,1,1-trifluorohex-5-en-2-yl)-1,3,4-oxadiazol-2-yl)-6-(((R)-pent-4-en-2-yl)oxy)-5-(trifluoromethyl)pyridin-3-yl)carbamate (48)

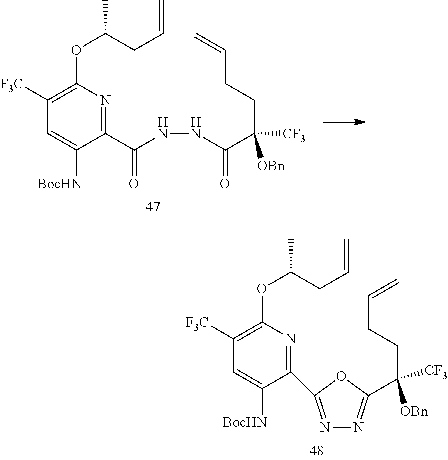 [SMILES: C=CCC[C@@](OCc1ccccc1)(C(=O)NNC(=O)c1nc(O[C@H](C)CC=C)c(C(F)(F)F)cc1NC(=O)OC(C)(C)C)C(F)(F)F ; C=CCC[C@@](OCc1ccccc1)(c1nnc(-c2nc(O[C@H](C)CC=C)c(C(F)(F)F)cc2NC(=O)OC(C)(C)C)o1)C(F)(F)F]

tert-Butyl (2-(2-((R)-2-(benzyloxy)-2-(trifluoromethyl)hex-5-enoyl)hydrazine-1-carbonyl)-6-(((R)-pent-4-en-2-yl)oxy)-5-(trifluoromethyl)pyridin-3-yl)carbamate (20 g, 29.645 mmol, 1 equiv.), DIPEA (11.494 g, 15.491 mL, 0.742 g/mL, 88.936 mmol, 3 equiv.) and 1,4-diazabicyclo[2.2.2]octane (0.333 g, 2.965 mmol, 0.1 equiv.) in CH₃CN (160 mL, 0.185 M, 8 Vols) at ambient temperature was added p-toluenesulfonyl chloride (7.6 g, 39.864 mmol, 1.345 equiv.). The reaction mixture was stirred at ambient until complete by LC analysis. Added water (100 mL) and MTBE (200 mL) to the mixture, stirred and phase split. The organic layer was washed with 1 M aq citric acid (100 mL×2) and aq satd. NaHCO₃ (200 mL). The organic layer was dried with Na₂SO₄, filtered, and concentrated via rotary evaporation to afford the title compound (21.26 g) as a viscous oil. ¹H NMR (400 MHz, DMSO) δ 9.71 (s, 1H), 8.86 (s, 1H), 7.46-7.26 (m, 5H), 5.91-5.72 (m, 2H), 5.24 (q, J=6.1 Hz, 1H), 5.14-4.98 (m, 4H), 4.78-4.64 (m, 2H), 2.44-2.27 (m, 3H), 1.49 (s, 9H), 1.32 (d, J=6.2 Hz, 3H), 1.28-1.22 (m, 3H) ppm. ESI-MS m/z calc. 656.24, found 657.22 (M+1)⁺.

### Step 7: tert-Butyl (2-(5-((R)-2-(benzyloxy)-1,1,1-trifluorohex-5-en-2-yl)-1,3,4-oxadiazol-2-yl)-6-(((R)-pent-4-en-2-yl)oxy)-5-(trifluoromethyl)pyridin-3-yl)(tert-butoxycarbonyl)carbamate (49)

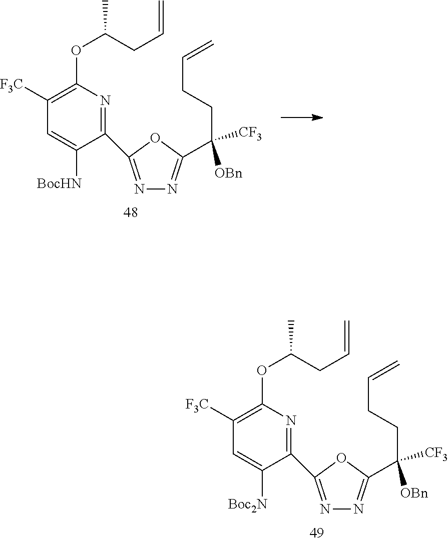

To a solution of tert-butyl (2-(5-((R)-2-(benzyloxy)-1,1,1-trifluorohex-5-en-2-yl)-1,3,4-oxadiazol-2-yl)-6-(((R)-pent-4-en-2-yl)oxy)-5-(trifluoromethyl)pyridin-3-yl)carbamate (19.466 g, 29.645 mmol, 1 equiv.) in toluene (116.794 mL, 0.254 M, 6 Vols) was added 4-(dimethylamino)pyridine (0.29 g, 2.372 mmol, 0.08 equiv.) followed by a solution of Boc₂O (7.764 g, 35.574 mmol, 1.2 equiv.) in toluene (19.466 mL, 1.523 M, 1 Vols) dropwise via addition funnel. The reaction mixture was stirred at ambient until complete reaction by LC analysis. Added water (100 mL, 5 vols) to the mixture, stirred for 10 min, phase split, remove the aqueous layer. Added 10% aq w/v KHSO₄ (4.217 g, 42.172 mL, 30.97 mmol, 1.045 equiv.) to the organic layer, stirred, phase split, removed the aqueous layer. The organic layer was washed with brine (80 mL, 4 vols), dried with Na₂SO₄ and filtered. The filtrate was then filtered through a silica gel pad (20 g, 230-400 mesh silica). The filtrate was concentrated via rotary evaporation and was used as is in the next step.

### Step 8: tert-Butyl ((4R,10R)-10-(benzyloxy)-4-methyl-25,10-bis(trifluoromethyl)-3-oxa-1(2,5)-oxadiazola-2(2,6)-pyridinacyclodecaphan-6-en-23-yl)(tert-butoxycarbonyl)carbamate (50)

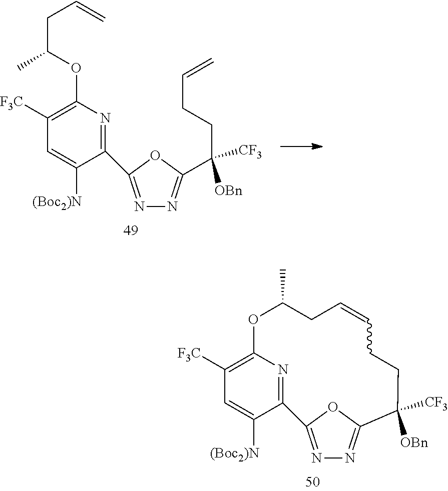

A solution of tert-butyl (2-(5-((R)-2-(benzyloxy)-1,1,1-trifluorohex-5-en-2-yl)-1,3,4-oxadiazol-2-yl)-6-(((R)-pent-4-en-2-yl)oxy)-5-(trifluoromethyl)pyridin-3-yl)(tert-butoxycarbonyl)carbamate (64.8 g, 84.95 mmol) in toluene (9,642 mL) was degassed using nitrogen with a gas dispersion tube in a 20 L reactor. Mixture was heated to 75° C. while continuing sub-surface N₂ sparge and added Umicore M101 Ru-catalyst (CAS #250220-36-1, 12.0 g, 12.76 mmol, 0.15 eq). After complete reaction, as determined by LC analysis, the mixture was cooled to 45° C. Then added 2-sulfanylpyridine-3-carboxylic acid (13.2 g, 85.07 mmol) followed by triethylamine (11.9 mL, 85.38 mmol). After stirring overnight at 45° C., mixture was cooled to ambient and then silica gel (170 g) was added to the mixture. The resulting slurry was stirred at room temp for 5 hours and then filtered through a Celite pad. The filtrate was concentrated and then diluted in a mixture of 80 mL DCM and 120 mL hexanes. The mixture was then loaded onto a 1.5 kg Gold ISCO silica column (CV=2400 mL, 600 mL/min) and purified with a gradient of 2% to 9% ethyl acetate/hexanes over 32 minutes (8 CV), then 9% ethyl acetate. All fractions containing tert-butyl ((4R,10R)-10-(benzyloxy)-4-methyl-25,10-bis(trifluoromethyl)-3-oxa-1(2,5)-oxadiazola-2(2,6)-pyridinacyclodecaphan-6-en-23-yl)(tert-butoxycarbonyl)carbamate were combined and concentrated in vacuo to afford 37.94 g (50% yield) of the title compound. ¹H NMR (400 MHz, DMSO) δ 8.57 (d, J=14.4 Hz, 1H), 7.40-7.27 (m, 5H), 5.51 (q, J=5.6 Hz, 2H), 5.07 (q, J=5.2 Hz, 1H), 4.63-4.49 (m, 2H), 2.56 (t, J=9.0 Hz, 1H), 2.34 (s, 2H), 2.13 (s, 1H), 1.49 (d, J=6.3 Hz, 3H), 1.35 (s, 2H), 1.28 (s, 15H), 1.25 (t, J=4.0 Hz, 3H) ppm. ESI-MS m/z calc. 728.26, found 729.31 (M+1)⁺.

### Step 9: tert-Butyl (tert-butoxycarbonyl)((4R,10R)-10-hydroxy-4-methyl-25,10-bis(trifluoromethyl)-3-oxa-1(2,5)-oxadiazola-2(2,6)-pyridinacyclodecaphane-23-yl)carbamate (51)

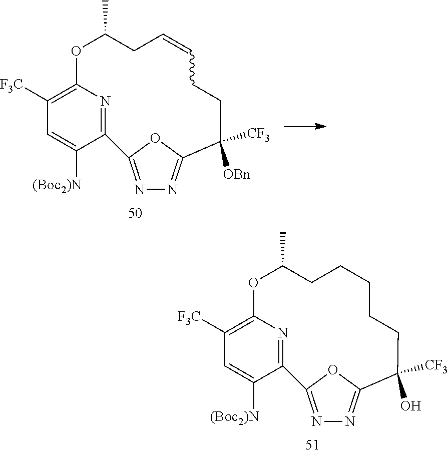

tert-Butyl ((4R,10R)-10-(benzyloxy)-4-methyl-25,10-bis(trifluoromethyl)-3-oxa-1(2,5)-oxadiazola-2(2,6)-pyridinacyclodecaphan-6-en-23-yl)(tert-butoxycarbonyl)carbamate (37.8 g, 42.537 mmol, 1 equiv.) was dissolved in EtOH (200 mL) and added to a reactor. Pd/C (10% loading, 50% wet palladium; Evonik NOBLYST® P1173, 6.292 g) was diluted in EtOH (400 mL, 0.106 M, 10.582 Vols) to create a slurry and that slurry was added to the reactor. The reactor atmosphere was purged by flushing 4 times with nitrogen and evacuating by vacuum, then flushing 4 times with hydrogen and evacuating by vacuum. The mixture was stirred (rpm=500-1000) at 25° C. under hydrogen atmosphere (pressure was ˜2.0 bar) for 24 hours. The mixture was cycled 3 times between vacuum and nitrogen, filtered over Celite, washed with ethanol (200 mL×4), and the filtrate was concentrated in vacuo to afford a foam-like residue. Heptane (200 mL) was added and the resulting mixture was rotated at 35° C. on a rotary evaporator with little-to-no vacuum which yielded a slurry. After concentrating in vacuo (40° C. bath temp., 10 mbar), an off-white solid was obtained. Heptane (150 mL) was added and the resulting slurry was stirred at ambient temperature overnight. The solid was collected by filtration, rinsed with heptane (30 mL), and dried in vacuum oven at 40° C. with N₂ bleed overnight to afford 27.7 g of tert-butyl (tert-butoxycarbonyl)((4R,10R)-10-hydroxy-4-methyl-25,10-bis(trifluoromethyl)-3-oxa-1(2,5)-oxadiazola-2(2,6)-pyridinacyclodecaphane-23-yl)carbamate. ¹H NMR (400 MHz, DMSO) δ 8.55 (s, 1H), 7.68 (s, 1H), 5.05 (ddd, J=9.3, 6.2, 2.5 Hz, 1H), 2.43 (s, 1H), 2.27 (t, J=11.1 Hz, 1H), 2.13 (dt, J=14.6, 7.4 Hz, 1H), 1.70 (s, 2H), 1.56-1.40 (m, 7H), 1.40-1.19 (m, 19H) ppm. ESI-MS m/z calc. 640.23, found 641.49 (M+1)⁺.

### Step 10: (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol (Compound I)

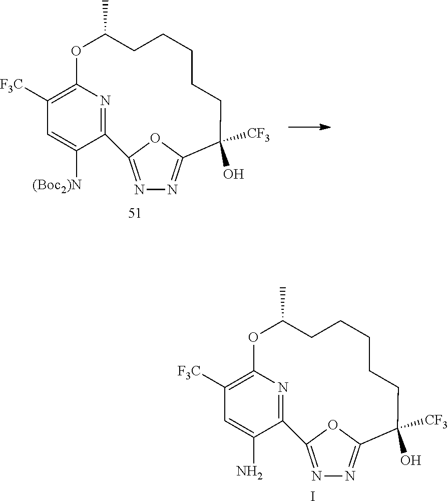

tert-Butyl (tert-butoxycarbonyl)((4R,10R)-10-hydroxy-4-methyl-25,10-bis(trifluoromethyl)-3-oxa-1(2,5)-oxadiazola-2(2,6)-pyridinacyclodecaphane-23-yl)carbamate (26.7 g, 38.346 mmol, 1 equiv.) was dissolved in DCM (213.6 mL, 0.18 M, 8 Vols), cooled in an ice bath and treated with trifluoroacetic acid (100.565 g, 67.493 mL, 1.49 g/mL, 881.969 mmol, 23 equiv.) under nitrogen, which resulted in a slight exotherm. Removed the ice bath, and stirred the yellow solution at ambient temperature until complete consumption of tert-butyl (tert-butoxycarbonyl)((4R,10R)-10-hydroxy-4-methyl-25,10-bis(trifluoromethyl)-3-oxa-1(2,5)-oxadiazola-2(2,6)-pyridinacyclodecaphane-23-yl)carbamate as determined by LC analysis. After concentrating, added DCM (10 vols), washed with cold sat. NaHCO₃ aq (100 mL×3), dried over Na₂SO₄, filtered, and concentrated on rotary evaporator. After removing solvent, the crude (37.8 g) was dissolved in 100 mL of DCM (3.75 vols), added 500 mL of heptane (18.73 vols) slowly to the solution, the solution became cloudy, slurry formed, the slurry became thicker, stirred at ambient overnight. The solid was collected by filtration, rinsed with heptane (70 mL) and dried in the vacuum oven at 40° C. with nitrogen bleed overnight to afford 14.58 g of (4R,10R)-23-amino-4-methyl-25,10-bis(trifluoromethyl)-3-oxa-1(2,5)-oxadiazola-2(2,6)-pyridinacyclodecaphan-10-ol as an off-white solid. After standing overnight, the mother liquor was filtered and the isolated solid was dried in the open air, then dried under vacuum at 40° C. with nitrogen bleed overnight affording an additional 0.58 g of (4R,10R)-23-amino-4-methyl-25,10-bis(trifluoromethyl)-3-oxa-1(2,5)-oxadiazola-2(2,6)-pyridinacyclodecaphan-10-ol as 2nd crop. Continued to dry under vacuum at 40-45° C. with N₂ bleed overnight. ¹H NMR (400 MHz, CDCl₃) δ 7.77 (s, 1H), 7.59 (s, 1H), 6.36 (s, 2H), 4.86-4.74 (m, 1H), 2.29 (t, J=11.9 Hz, 1H), 2.11 (dt, J=14.7, 7.6 Hz, 1H), 1.74 (s, 2H), 1.57-1.41 (m, 4H), 1.35 (d, J=6.3 Hz, 3H), 1.25 (d, J=2.4 Hz, 1H), 1.22-1.13 (m, 1H) ppm. ESI-MS m/z calc. 440.13, found 441.32 (M+1)⁺.

### Example 25: Alternative Synthesis of (6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol (Compound I)

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (52)

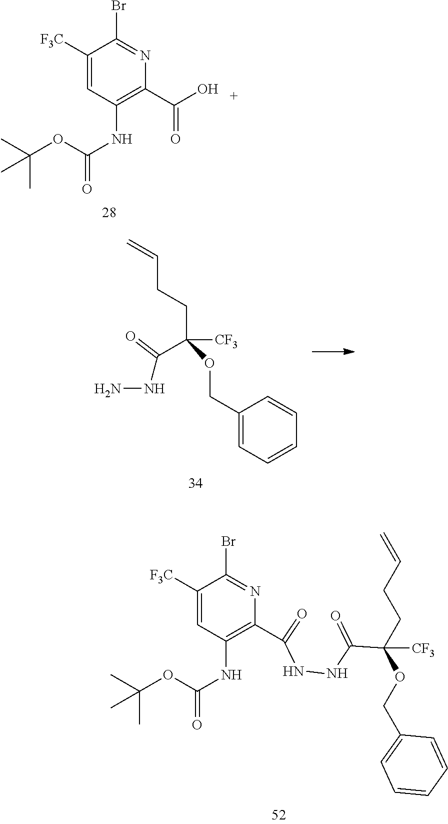 [SMILES: CC(C)(C)OC(=O)Nc1cc(C(F)(F)F)c(Br)nc1C(=O)O ; C=CCC[C@@](OCc1ccccc1)(C(=O)NN)C(F)(F)F ; C=CCC[C@@](OCc1ccccc1)(C(=O)NNC(=O)c1nc(Br)c(C(F)(F)F)cc1NC(=O)OC(C)(C)C)C(F)(F)F]

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T3P (622 g of 50% w/w, 977.4 mmol) using an ice-water bath to keep the temperature <35° C. (temperature rose to 34° C.) and the resulting mixture was stirred at ambient temperature for 18 h. Added additional DIEA (100 mL, 574.1 mmol) and T3P (95 g, 298.6 mmol) and stirred at ambient temperature for 2 days. Starting material was still observed and an additional T3P (252 g, 792 mmol) was added and stirred for 5 days. The reaction was quenched with the slow addition of water (2.5 L) and the mixture stirred for 30 min. The organic phase was separated, and the aqueous phase extracted with EtOAc (2 L). The combined organic phases were washed with brine, dried over MgSO₄, filtered, and concentrated in vacuo. The crude product was dissolved in MTBE (300 mL) and diluted with heptane (3 L), the mixture stirred at ambient temperature for 12 h affording a light yellow slurry. The slurry was filtered, and the resultant solid was air dried for 2 h, then in vacuo at 40° C. for 48 h. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/mm, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (53)

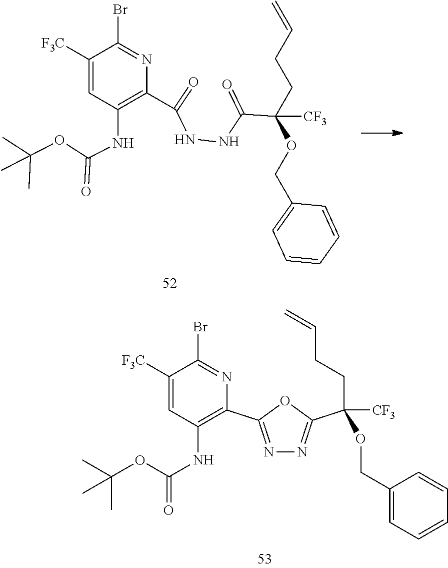 [SMILES: C=CCC[C@@](OCc1ccccc1)(C(=O)NNC(=O)c1nc(Br)c(C(F)(F)F)cc1NC(=O)OC(C)(C)C)C(F)(F)F ; C=CCC[C@@](OCc1ccccc1)(c1nnc(-c2nc(Br)c(C(F)(F)F)cc2NC(=O)OC(C)(C)C)o1)C(F)(F)F]

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 h. The mixture was stirred at 70° C. for 9 h then additional p-toluenesulfonyl chloride (6.5 g, 34.09 mmol) was added. The mixture was stirred for a total of 24 h then allowed to cool to ambient temperature. Acetonitrile was removed in vacuo affording a dark orange oil which was diluted with EtOAc (1.5 L) and water (1.5 L). The organic phase was separated and washed with 500 mL of 1 M HCl, 500 mL of brine, dried over MgSO₄, filtered, and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 3: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (54)

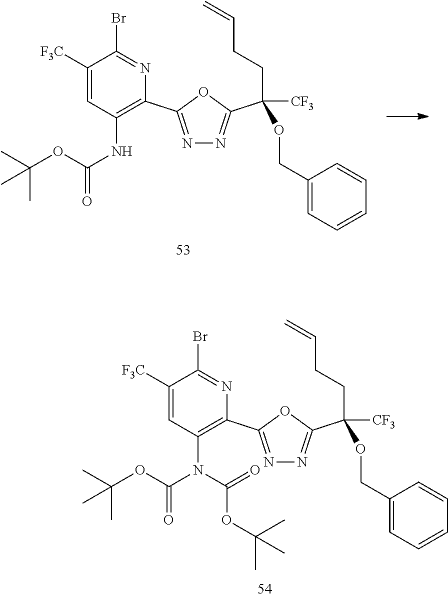 [SMILES: C=CCC[C@@](OCc1ccccc1)(c1nnc(-c2nc(Br)c(C(F)(F)F)cc2NC(=O)OC(C)(C)C)o1)C(F)(F)F ; C=CCC[C@@](OCc1ccccc1)(c1nnc(-c2nc(Br)c(C(F)(F)F)cc2N(C(=O)OC(C)(C)C)C(=O)OC(C)(C)C)o1)C(F)(F)F]

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed by DMAP (2.09 g, 17.11 mmol). Added a solution of di-tert-butyl dicarbonate (111.6 g, 511.3 mmol) in MTBE (250 mL) over approx. 8 minutes, and the resulting mixture was stirred for additional 30 min. Added 1 L of water and separated the layers. The organic layer was washed with KHSO₄ (886 mL of 0.5 M, 443.0 mmol), 300 mL brine, dried with MgSO₄ and most (>95%) of the MTBE was evaporated by rotary evaporation at 45° C., leaving a thick oil. Added 1.125 L of heptane, spun in the 45° C. rotary evaporator bath until dissolved, then evaporated out 325 mL of solvent by rotary evaporation. The rotary evaporator bath temperature was allowed to cool to room temperature and crystallization was observed during evaporation. The flask was then stored in a freezer (−20° C.) overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m/z calc. 750.14874, found 751.1 (M+1)⁺; retention time: 3.76 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 4: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (55)

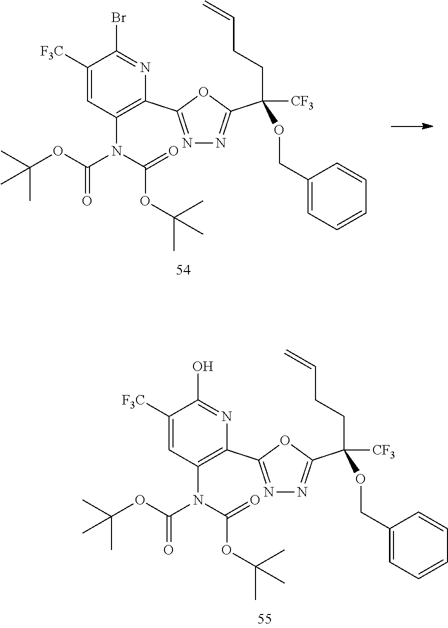 [SMILES: C=CCC[C@@](OCc1ccccc1)(c1nnc(-c2nc(Br)c(C(F)(F)F)cc2N(C(=O)OC(C)(C)C)C(=O)OC(C)(C)C)o1)C(F)(F)F ; C=CCC[C@@](OCc1ccccc1)(c1nnc(-c2nc(O)c(C(F)(F)F)cc2N(C(=O)OC(C)(C)C)C(=O)OC(C)(C)C)o1)C(F)(F)F]

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 h. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated, and the organic phase washed with water (3×3 L) and brine (1×2.5 L). The organic phase was dried with MgSO₄, filtered, and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotary evaporator at 100 mbar pressure at room temperature for 1.5 h. The solid mass was stirred mechanically for 2 h at room temperature, and the resulting fine suspension was filtered, washed with dry, ice-cold heptane, and dried under vacuum at 45° C. with a nitrogen bleed for 16 h to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d₆) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m/z calc. 688.23315, found 689.0 (M+1)⁺; retention time: 3.32 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 5: [(3R)-3-[[6-[5-[1-Benzyloxy-1-(trifluoromethyl)pent-4-enyl]-1,3,4-oxadiazol-2-yl]-5-[bis(tert-butoxycarbonyl)amino]-3-(trifluoromethyl)-2-pyridyl]oxy]butyl]-triphenyl-phosphonium; iodide (56)

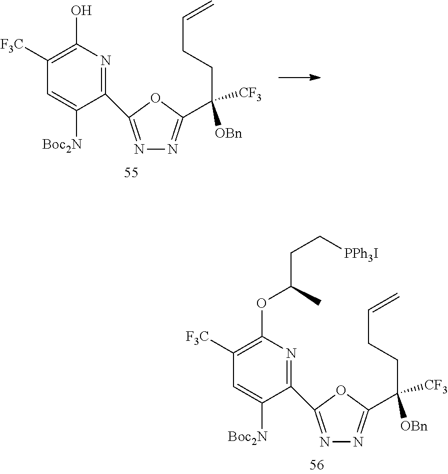

tert-Butyl N-[2-[5-[1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (850 mg, 1.1726 mmol) was dissolved in THF (6 mL) at RT. [(3S)-3-Hydroxybutyl]-triphenyl-phosphoniumiodide (550 mg, 1.1897 mmol) was added, followed by PPh₃ (868 mg, 3.3094 mmol). The cloudy mixture was stirred under a N₂ balloon. DIAD (678.60 mg, 0.65 mL, 3.3560 mmol) was added dropwise. Reaction was exothermic so the rate of addition was important to keep the reaction temperature <40° C. The mixture was then stirred for 60 h at RT. The mixture was concentrated to remove most THF. The residue was treated with ether (20 ml) and hexanes (10 ml) and sonicated. After settling for 5 min, LC/MS analysis showed most desired product was in the yellowish precipitate, while the solution contained mostly PPh₃ oxide, unreacted starting material, and other byproducts. The precipitate was purified by reverse phase HPLC (Higgins Analytical, 30 mm×100 mm, 30 to 70% B, A=Water (0.1% HCl), B=Acetonitrile (0.10% HCl), 40 m, 25 mL/min). Fractions were combined and concentrated before partitioned between EtOAc (40 ml) and NaHCO₃ (30 ml, sat. aq.). The EtOAc layer was dried over anh. MgSO₄, filtered and concentrated to afford [(3R)-3-[[6-[5-[1-benzyloxy-1-(trifluoromethyl)pent-4-enyl]-1,3,4-oxadiazol-2-yl]-5-[bis(tert-butoxycarbonyl)amino]-3-(trifluoromethyl)-2-pyridyl]oxy]butyl]-triphenyl-phosphonium; iodide (680 mg, 49%) as a white foam. ESI-MS m/z calc. 1132.2836, found 1006.1 (M−127)⁺; retention time: 8.12 minutes; LCMS Method: Merckmillipore Chromolith SpeedROD C18 column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

### Step 6: [(3R)-3-[[6-[5-[1-Benzyloxy-4-oxo-1-(trifluoromethyl)butyl]-1,3,4-oxadiazol-2-yl]-5-[bis(tert-butoxycarbonyl)amino]-3-(trifluoromethyl)-2-pyridyl]oxy]butyl]-triphenyl-phosphonium; iodide (57)

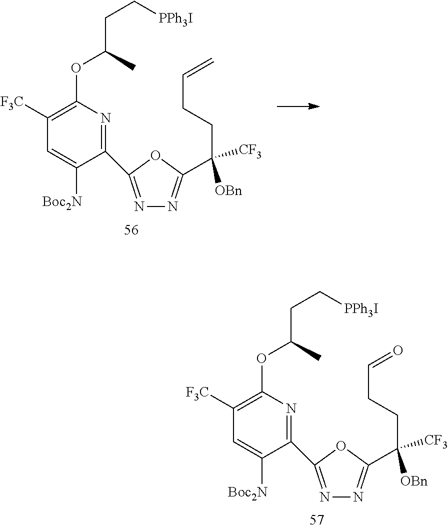

[(3R)-3-[[6-[5-[1-Benzyloxy-1-(trifluoromethyl)pent-4-enyl]-1,3,4-oxadiazol-2-yl]-5-[bis(tert-butoxycarbonyl)amino]-3-(trifluoromethyl)-2-pyridyl]oxy]butyl]-triphenyl-phosphonium; iodide (150 mg, 0.1258 mmol) was taken into a mixed solvents of THF (1 mL) and water (0.5 mL) at RT. Sodium periodate (99 mg, 0.4629 mmol) was added, followed by osmium tetroxide in t-butanol (7.6757 mg, 0.0095 mL of 2.5% w/w, 754.80 nmol). The colorless mixture turned milky after 10 min. The mixture was stirred at RT for 5 h. The crude mixture was passed through silica gel pad and the filtrate was concentrated to afford [(3R)-3-[[6-[5-[1-benzyloxy-4-oxo-1-(trifluoromethyl)butyl]-1,3,4-oxadiazol-2-yl]-5-[bis(tert-butoxycarbonyl)amino]-3-(trifluoromethyl)-2-pyridyl]oxy]butyl]-triphenyl-phosphonium; iodide (85 mg, 51%) as a pale yellow semi-solid. ESI-MS m/z calc. 1134.2628, found 1007.9 (M−127)⁺; retention time: 7.5 minutes. LCMS Method: Merckmillipore Chromolith SpeedROD C18 column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

### Step 7: tert-Butyl N-[(9Z,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]carbamate; tert-butyl N-[(9Z,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]carbamate (58)

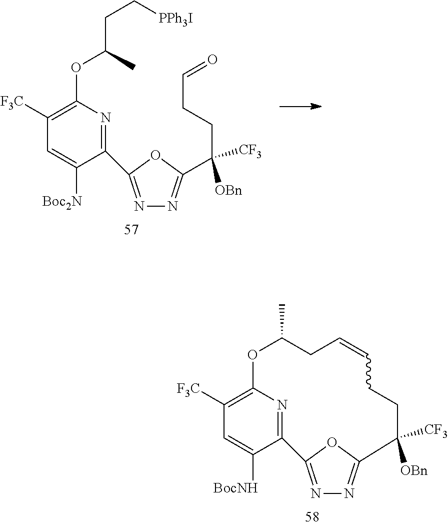

[(3R)-3-[[6-[5-[1-Benzyloxy-4-oxo-1-(trifluoromethyl)butyl]-1,3,4-oxadiazol-2-yl]-5-[bis(tert-butoxycarbonyl)amino]-3-(trifluoromethyl)-2-pyridyl]oxy]butyl]-triphenyl-phosphonium; iodide (200 mg, 0.1674 mmol) was dissolved in THF (15 mL) at RT. Sodium t-butoxide (54 mg, 0.5619 mmol) was added in one portion. The mixture was stirred under a N₂ balloon for 60 h. LC/MS analysis showed product formation, plus multiple byproducts. The mixture was combined with a mixture following the same procedure (except run on ⅕ of the scale of this reaction) and partitioned between EtOAc (40 ml) and water (20 ml). The EtOAc layer was washed with brine, dried over anhydrous magnesium sulfate, filtered and concentrated. The residue was purified by silica gel chromatography (40 g column), using 0-10% EtOAc in Hexanes to afford tert-butyl N-[(9Z,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]carbamate (55 mg of both cis and trans isomers, 50%) as colorless oil. ESI-MS m/z calc. 628.212, found 629.6 (M+1)⁺; Retention time: 8.93 minutes and ESI-MS m/z calc. 628.212, found 629.6 (M+1)⁺; Retention time: 9.13 minutes. LCMS Method: Merckmillipore Chromolith SpeedROD C18 column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

### Step 8: tert-Butyl ((4R,10R)-10-hydroxy-4-methyl-25,10-bis(trifluoromethyl)-3-oxa-1(2,5)-oxadiazola-2(2,6)-pyridinacyclodecaphane-23-yl)carbamate (59)

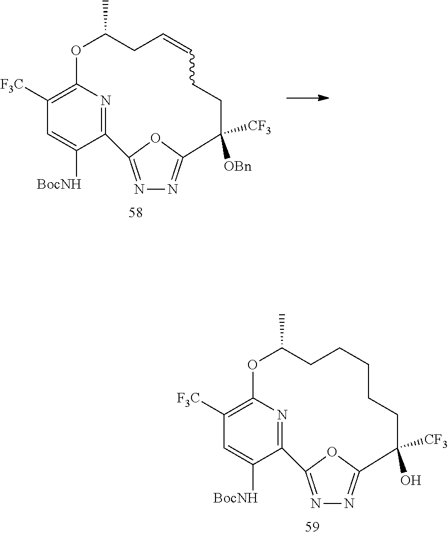 [SMILES: C[C@@H]1CC=CCC[C@](OCc2ccccc2)(C(F)(F)F)c2nnc(o2)-c2nc(c(C(F)(F)F)cc2NC(=O)OC(C)(C)C)O1 ; C[C@@H]1CCCCC[C@](O)(C(F)(F)F)c2nnc(o2)-c2nc(c(C(F)(F)F)cc2NC(=O)OC(C)(C)C)O1]

tert-Butyl ((4R,10R)-10-hydroxy-4-methyl-25,10-bis(trifluoromethyl)-3-oxa-1(2,5)-oxadiazola-2(2,6)-pyridinacyclodecaphane-23-yl)carbamate is prepared from tert-butyl N-[(9Z,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]carbamate; tert-butyl N-[(9Z,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]carbamate in a manner analogous to Step 9 of Example 24.

### Step 9: (6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol (Compound I)

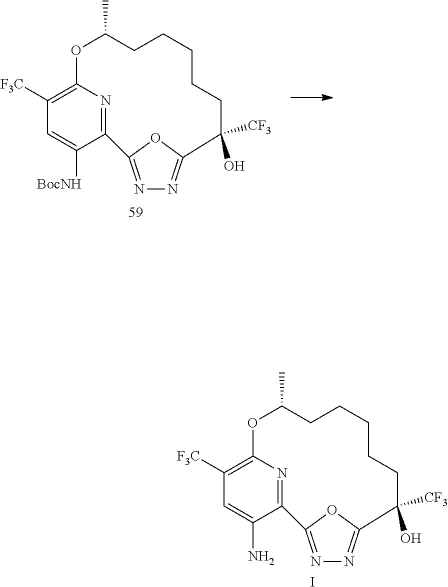

Compound I is prepared from tert-butyl ((4R,10R)-10-hydroxy-4-methyl-25,10-bis(trifluoromethyl)-3-oxa-1(2,5)-oxadiazola-2(2,6)-pyridinacyclodecaphane-23-yl)carbamate in a manner analogous to Step 10 of Example 24.

### Example 26: Preparation of (6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol (Compound I)

### Step 1: tert-Butyl (6-(benzyloxy)-2-(2-((2R,8S)-2-(benzyloxy)-8-((tert-butyldiphenylsilyl)oxy)-2-(trifluoromethyl)nonanoyl)hydrazine-1-carbonyl)-5-(trifluoromethyl)pyridin-3-yl)carbamate (60)

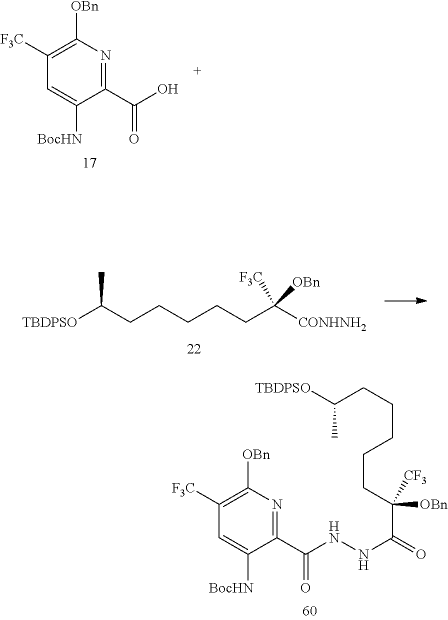

In a 250 mL RBF 6-(benzyloxy)-3-((tert-butoxycarbonyl)amino)-5-(trifluoromethyl)picolinic acid (5.39 g, 13.071 mmol, 1 equiv.) and (2R,8S)-2-(benzyloxy)-8-((tert-butyldiphenylsilyl)oxy)-2-(trifluoromethyl)nonanehydrazide (8.638 g, 14.378 mmol, 1.1 equiv.) in 2-MeTHF (35.035 mL, 6.5 Vols) was added 2-chloro-4,6-dimethoxy-1,3,5-triazine (CDMT, 2.639 g, 15.032 mmol, 1.15 equiv.). The reaction mixture was stirred for NLT 15 min, 4-methylmorpholine (1.653 g, 1.796 mL, 0.92 g/mL, 16.33 mmol, 1.25 equiv.) was added. The reaction was stirred at ambient until complete by LC analysis (typically 1 h or less). The reaction mixture was diluted with 2-MeTHF (100 mL), added 50 mL of water, stirred, phase split. The organic layer was washed with aq. sat. NaHCO₃ (30 mL×2), brine (30 mL), dried over Na₂SO₄ and concentrated. The crude was purified by column chromatograph (330 g silica) eluting with EtOAc/Hexane (0-10%), clean fractions were collected, concentrated and solvent swap with heptane. The obtained solid was dried under vacuum oven at 40° C. with nitrogen bleed overnight affording 11.41 g as an off-white solid. ¹H NMR (400 MHz, DMSO) δ 10.88-10.65 (m, 2H), 10.30 (s, 1H), 9.09 (s, 1H), 7.60 (dt, J=6.7, 1.7 Hz, 4H), 7.52-7.28 (m, 16H), 5.67 (s, 2H), 4.88-4.71 (m, 2H), 3.81 (p, J=5.9 Hz, 1H), 2.21-2.00 (m, 2H), 1.45 (s, 11H), 1.37 (d, J=6.5 Hz, 2H), 1.32-1.19 (m, 4H, corrected for heptane), 1.04-0.94 (m, 12H) ppm. ESI-MS m/z calc. 994.41, found 995.37 (M+1)⁺.

### Step 2: tert-Butyl (6-(benzyloxy)-2-(5-((2R,8S)-2-(benzyloxy)-8-((tert-butyldiphenylsilyl)oxy)-1,1,1-trifluorononan-2-yl)-1,3,4-oxadiazol-2-yl)-5-(trifluoromethyl)pyridin-3-yl)carbamate (61)

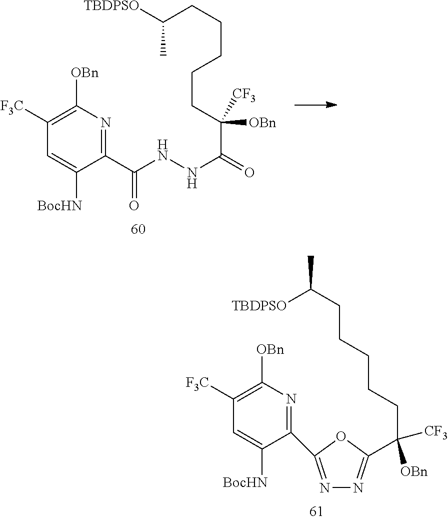

In a 250 mL RBF, tert-butyl (6-(benzyloxy)-2-(2-((2R,8S)-2-(benzyloxy)-8-((tert-butyldiphenylsilyl)oxy)-2-(trifluoromethyl)nonanoyl)hydrazine-1-carbonyl)-5-(trifluoromethyl)pyridin-3-yl)carbamate (5.664 g, 5.692 mmol, 1 equiv.), N,N-diisopropylethylamine (2.207 g, 2.974 mL, 0.742 g/mL, 17.075 mmol, 3 equiv.) and 1,4-diazabicyclo[2.2.2]octane (63.845 mg, 0.569 mmol, 0.1 equiv.) in CH₃CN (45.312 mL, 8 Vols) stirred at ambient added p-toluenesulfonyl chloride (1.459 g, 7.654 mmol, 1.345 equiv.). The mixture was stirred at ambient until complete by LC analysis (approximately 2 hours). The mixture was concentrated and IPAc (10 vols) was added to the mixture, followed by aq. 10% w/v KHSO₄ aq. (25 mL). The resulting mixture was stirred vigorously for 30 min, repeated until pH of aqueous layer was ˜2 (pH paper). To the organic layer was added 0.5 M aq NaOH (25 mL) and stirred vigorously for 1 hr. The organic layer was isolated and washed with brine/water (1:1, 20 mL×2), dried over Na₂SO₄, filtered, concentrated, diluted with heptane, and then dried on house vacuum at ambient overnight affording ˜6 g product as a dark amber oil. The crude material was used in the next step without further purification. ¹H NMR (400 MHz, DMSO) δ 9.75 (s, 1H), 8.88 (s, 1H), 7.62-7.54 (m, 4H), 7.47-7.25 (m, 16H), 5.49 (s, 2H), 4.76-4.63 (m, 2H), 3.79 (q, J=5.8 Hz, 1H), 2.46-2.23 (m, 2H), 1.48 (s, 11H), 1.32-1.18 (m, 6H, corrected for heptane), 1.01-0.93 (m, 12H) ppm. ESI-MS m/z calc. 976.40, found 977.41 (M+1)⁺.

### Step 3: tert-Butyl (6-(benzyloxy)-2-(5-((2R,8S)-2-(benzyloxy)-1,1,1-trifluoro-8-hydroxynonan-2-yl)-1,3,4-oxadiazol-2-yl)-5-(trifluoromethyl)pyridin-3-yl) arbamate (67)

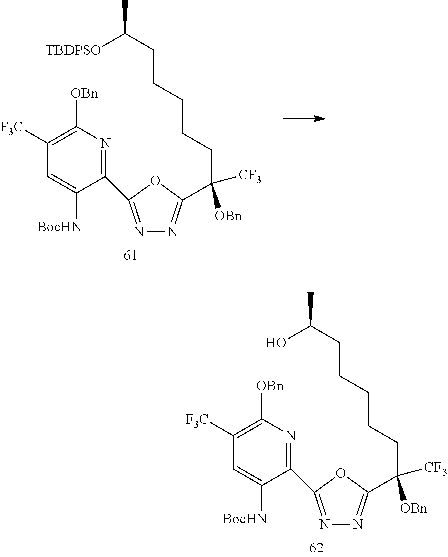

In a 20 mL vial, tert-butyl (6-(benzyloxy)-2-(5-((2R,8S)-2-(benzyloxy)-8-((tert-butyldiphenylsilyl)oxy)-1,1,1-trifluorononan-2-yl)-1,3,4-oxadiazol-2-yl)-5-(trifluoromethyl)pyridin-3-yl)carbamate (1.2 g, 1.228 mmol, 1 equiv.) in THF (3.6 mL, 3 Vols) was added tetrabutylammonium fluoride (1.0 M solution in THF, 1.842 mL, 1.842 mmol, 1.5 equiv.). The reaction mixture was stirred at ambient until complete reaction. After stirring ˜48 h, the conversion was 98.6% (AUC). The mixture was concentrated and was dissolved in MTBE (24 mL). Added aq satd. NH₄Cl (15 mL), stirred for 15 min, phase separated, repeated. The organic layer was washed with brine/water (1:1, 15 mL). The organic layer was dried over Na₂SO₄, filtered, and concentrated. The crude was purified by column (40 g, silica) chromatography eluting with EtOAc/hexane (0-20% EtOAc over 18 min), the product eluted at 20% EtOAc/hexane. Product-containing fractions were collected, concentrated, solvent swap with heptane, dried under house vacuum at ambient overnight affording 0.7 g as a viscous oil. ¹H NMR (400 MHz, DMSO) δ 9.74 (s, 1H), 8.88 (s, 1H), 7.51-7.39 (m, 4H), 7.39-7.23 (m, 6H), 5.51 (s, 2H), 4.79-4.63 (m, 2H), 4.30 (d, J=4.7 Hz, 1H), 3.54 (p, J=5.6 Hz, 1H), 2.48-2.32 (m, 2H), 1.49 (s, 11H), 1.41-1.20 (m, 6H, corrected for presence of residual heptane), 1.00 (d, J=6.1 Hz, 3H) ppm. ESI-MS m/z calc. 738.29, found 739.37 (M+1)⁺.

### Step 4: tert-Butyl (2-(5-((2R,8S)-2-(benzyloxy)-1,1,1-trifluoro-8-hydroxynonan-2-yl)-1,3,4-oxadiazol-2-yl)-6-hydroxy-5-(trifluoromethyl)pyridin-3-yl)carbamate (63)

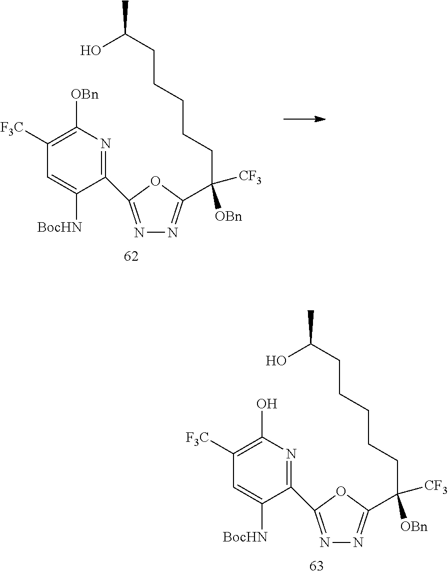 [SMILES: C[C@H](O)CCCCC[C@@](OCc1ccccc1)(c1nnc(-c2nc(OCc3ccccc3)c(C(F)(F)F)cc2NC(=O)OC(C)(C)C)o1)C(F)(F)F ; C[C@H](O)CCCCC[C@@](OCc1ccccc1)(c1nnc(-c2nc(O)c(C(F)(F)F)cc2NC(=O)OC(C)(C)C)o1)C(F)(F)F]

In a 50 mL RBF, charged tert-butyl (6-(benzyloxy)-2-(5-((2R,8S)-2-(benzyloxy)-1,1,1-trifluoro-8-hydroxynonan-2-yl)-1,3,4-oxadiazol-2-yl)-5-(trifluoromethyl)pyridin-3-yl)carbamate (0.677 g, 0.871 mmol, 1 equiv.) and EtOH (13.54 mL, 20 Vols). The solution was cycled 3 times between vacuum and N₂, added 10% Pd/C 50% w/w water wet palladium (Evonink NOBLYST®P1173, 0.322 g, 5% w/w Pd, 0.151 mmol, 0.174 equiv.) and the mixture cycled 3 times vacuum/hydrogen. The mixture was stirred at ambient under hydrogen balloon until complete conversion by LC analysis (typically less than 2 h). The mixture was cycled 3 times vacuum/N₂. The reaction mixture was filtered through Celite, filter cake washed with EtOH, and the filtrate was concentrated. The crude product was purified by column (24 g, silica) chromatography eluting with EtOAc/hexane (0-25% EtOAc over 18 min), the product eluted at 25% ethyl acetate and hexane. Product-containing fractions were collected, concentrated, solvent swap with heptane, and dried under house vacuum at ambient overnight affording tert-butyl (2-(5-((2R,8S)-2-(benzyloxy)-1,1,1-trifluoro-8-hydroxynonan-2-yl)-1,3,4-oxadiazol-2-yl)-6-hydroxy-5-(trifluoromethyl)pyridin-3-yl)carbamate (0.49 g, 86%) as a light-yellow foam. ¹H NMR (400 MHz, DMSO) δ 12.59 (s, 1H), 9.71 (s, 1H), 8.73 (s, 1H), 7.45-7.29 (m, 5H), 4.76-4.58 (m, 2H), 4.32 (s, 1H), 3.56 (d, J=7.6 Hz, 1H), 2.48-2.31 (m, 2H), 1.47 (s, 11H), 1.40-1.21 (m, 6H, corrected for presence of residual heptane), 1.02 (d, J=6.1 Hz, 3H) ppm. ESI-MS m z calc. 648.24, found 649.26 (M+1)⁺.

### Step 5: tert-Butyl ((4R,10R)-10-(benzyloxy)-4-methyl-25,10-bis(trifluoromethyl)-3-oxa-1(2,5)-oxadiazola-2(2,6)-pyridinacyclodecaphane-23-yl)carbamate (64)

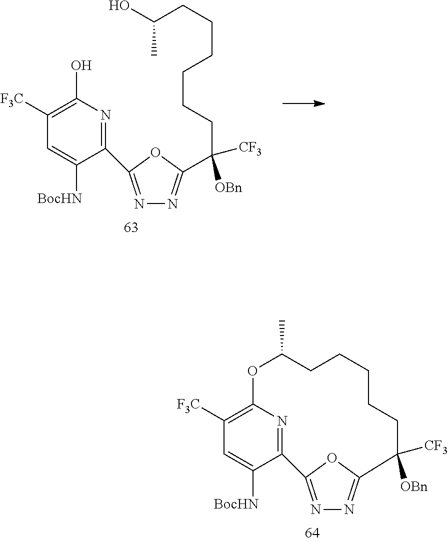 [SMILES: C[C@H](O)CCCCCC[C@@](OCc1ccccc1)(c1nnc(-c2nc(O)c(C(F)(F)F)cc2NC(=O)OC(C)(C)C)o1)C(F)(F)F ; C[C@@H]1CCCCC[C@](OCc2ccccc2)(C(F)(F)F)c2nnc(o2)-c2nc(c(C(F)(F)F)cc2NC(=O)OC(C)(C)C)O1]

To a solution of triphenylphosphine (118.889 mg, 0.453 mmol, 1.5 equiv.) in toluene (3.0 mL) cooled with an ice bath was added diisopropyl azodicarboxylate (93.053 mg, 0.091 mL, 1.027 g/mL, 0.453 mmol, 1.5 equiv.) in toluene (2 mL) keeping the temperature at 0-5° C. After stirring for 30 min tert-butyl N-(2-{5-[(2R,8S)-2-(benzyloxy)-1,1,1-trifluoro-8-hydroxynonan-2-yl]-1,3,4-oxadiazol-2-yl}-6-hydroxy-5-(trifluoromethyl) pyridin-3-yl) carbamate (200 mg, 0.302 mmol, 1 equiv.) in toluene (5 mL) was added drop wise through syringe. During the addition, the temperature was kept below 20° C. (17.8-18.5° C.). Total solvent volume was 10 mL (50 vols). After the addition was done, the mixture was stirred at ambient temperature Right after addition, LC found no starting compound was remained. (Title compound and dimer were observed, title compound:dimer=77:23.) Stopped the reaction after 30 min, the solvent was removed by rotary evaporator. Added toluene (1 mL), stirred, added heptane (1 mL), seeded with TPPO-DIAD and slurry appeared. Stirred at ambient overnight. The solid was filtered off. The filtrate was concentrated and purified by column (12 g HP silica) eluting with EtOAc/hexane (0-20% EtOAc). The product eluted at 5% EtOAc/hexane. Product-containing fractions were collected, concentrated in vacuo, and dried under house vacuum at ambient temperature overnight affording the title compound as an off-white solid (100 mg, 52% yield). ¹H NMR (400 MHz, DMSO) δ 9.30 (s, 1H), 8.83 (s, 1H), 7.46-7.34 (m, 5H), 4.97 (d, J=8.0 Hz, 1H), 4.79-4.57 (m, 2H), 2.67 (d, J=11.8 Hz, 1H), 2.36-2.21 (m, 1H), 1.86 (s, 1H), 1.76-1.62 (m, 2H), 1.56 (s, 12H), 1.46 (d, J=6.3 Hz, 3H), 1.31 (d, J=6.1 Hz, 2H) ppm. ESI-MS m/z calc. 630.23, found 631.20 (M+1)⁺.

### Step 6: tert-Butyl ((4R,10R)-10-hydroxy-4-methyl-2⁵,10-bis(trifluoromethyl)-3-oxa-1(2,5)-oxadiazola-2(2,6)-pyridinacyclodecaphane-2³-yl)carbamate (59)

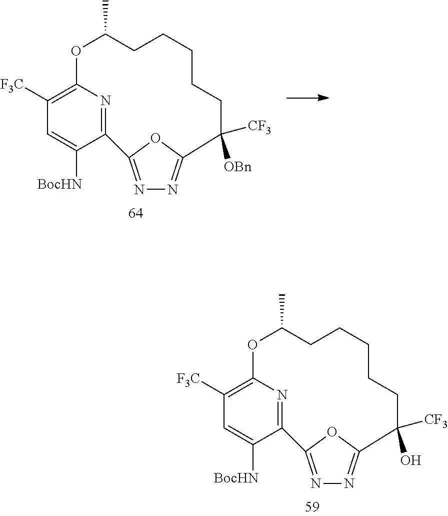 [SMILES: C[C@@H]1CCCCC[C@](OCc2ccccc2)(C(F)(F)F)c2nnc(o2)-c2nc(c(C(F)(F)F)cc2NC(=O)OC(C)(C)C)O1 ; C[C@@H]1CCCCC[C@](O)(C(F)(F)F)c2nnc(o2)-c2nc(c(C(F)(F)F)cc2NC(=O)OC(C)(C)C)O1]

To a mixture of tert-butyl ((4R,10R)-10-(benzyloxy)-4-methyl-25,10-bis(trifluoromethyl)-3-oxa-1(2,5)-oxadiazola-2(2,6)-pyridinacyclodecaphane-23-yl)carbamate (88 mg, 0.14 mmol, 1 equiv.) and EtOH (2 mL, 0.07 M, ˜22 volumes) was added Pd/C (10% loading, 50% water wet, 0.021 g). The vessel was flushed four times by purging the atmosphere with hydrogen and evacuating by vacuum. The mixture was stirred at ambient temperature under a hydrogen atmosphere (balloon). After complete consumption of tert-butyl ((4R,10R)-10-(benzyloxy)-4-methyl-25,10-bis(trifluoromethyl)-3-oxa-1(2,5)-oxadiazola-2(2,6)-pyridinacyclodecaphane-23-yl)carbamate was observed by LC, the flushed three times by purging the atmosphere with nitrogen and evacuating by vacuum. The resulting mixture was filtered through a pad of Celite, and the Celite pad and collected solids were rinsed with ethanol. The filtrate was concentrated via rotary evaporation and diluted with heptane. The resulting mixture was concentrated via rotary evaporation and this was repeated two times. The title compound was obtained as a solid (77 mg). ¹H NMR (400 MHz, DMSO) δ 9.25 (s, 1H), 8.69 (s, 1H), 7.68 (s, 1H), 4.92 (d, J=7.9 Hz, 1H), 2.26 (d, J=11.1 Hz, 1H), 2.19-2.06 (m, 1H), 1.67 (d, J=16.6 Hz, 2H), 1.54-1.36 (m, 16H, corrected for presence of residual heptane solvent), 1.24 (s, 2H) ppm. ESI-MS m/z calc. 540.18, found 541.25 (M+1)⁺.

### Step 7: (6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol (Compound I)

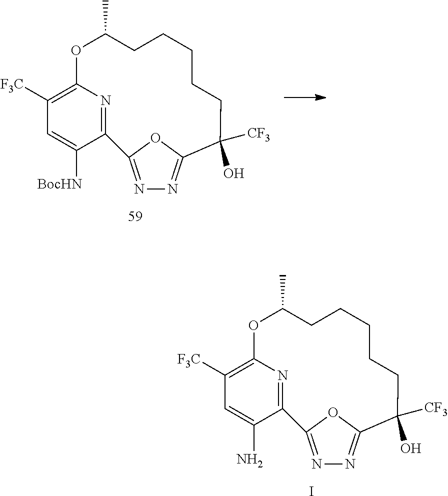

(6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol is synthesized from tert-butyl ((4R,10R)-10-hydroxy-4-methyl-2⁵,10-bis(trifluoromethyl)-3-oxa-1(2,5)-oxadiazola-2(2,6)-pyridinacyclodecaphane-2³-yl)carbamate in a manner analogous to Step 10 of Example 24.

### Example 27: Alternative Synthesis of tert-Butyl ((4S,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate (64)

### Step 1: tert-Butyl (2-(2-((2R,8S)-2-(benzyloxy)-8-((tert-butyldiphenylsilyl)oxy)-2-(trifluoromethyl)nonanoyl)hydrazine-1-carbonyl)-6-bromo-5-(trifluoromethyl)pyridin-3-yl)carbamate (65)

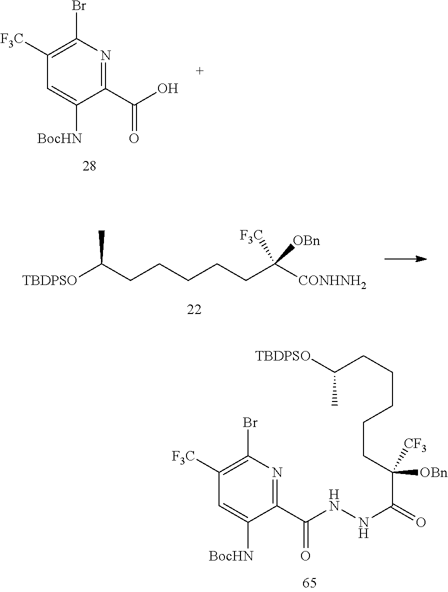

To a 250 mL RBF containing 6-bromo-3-((tert-butoxycarbonyl)amino)-5-(trifluoromethyl)picolinic acid (11.271 g, 29.265 mmol, 1 equiv.) and (2R,8S)-2-(benzyloxy)-8-((tert-butyldiphenylsilyl)oxy)-2-(trifluoromethyl)nonanehydrazide (20.22 g, 33.655 mmol, 1.15 equiv.) in ethyl acetate (90.17 mL, 8 Vols) was added 4-methylmorpholine (10.347 g, 11.271 mL, 0.918 g/mL, 102.296 mmol, 3.495 equiv.) followed by T3P (50% w/w in EtOAc, 24.21 g, 22.648 mL, 1.069 g/mL, 38.045 mmol, 1.3 equiv.). The mixture was stirred at ambient overnight, after which water (110 mL) was added to the mixture. After mixing, the organic phase was isolated and 110 mL of 5% aq. w/v KHSO₄ was added to the organic layer. After mixing, the organic phase was isolated and to it was added 110 mL of 5% aq. w/v KHCO₃. After mixing, the organic phase was isolated, dried over Na₂SO₄, filtered, and concentrated. The resulting residue was diluted with diluted with DCM, to which silica gel (40 g, 230-400 mesh) was added. The resulting mixture was loaded on silica gel pad and flushed with 10% EtOAc/hexane (1 L×3). The filtrate was concentrated, diluted with heptane, concentrated, and dried affording the title compound (20.21 g) as viscous oil. ¹H NMR (400 MHz, DMSO) δ 11.08 (s, 1H), 10.93 (s, 1H), 10.27 (s, 1H), 9.15 (s, 1H), 7.61 (dt, J=7.6, 1.8 Hz, 4H), 7.52-7.29 (m, 11H), 4.79 (q, J=11.4 Hz, 2H), 3.82 (q, J=5.9 Hz, 1H), 2.19-1.96 (m, 2H), 1.46 (s, 13H), 1.25 (d, J=2.5 Hz, 4H, corrected for presence of residual heptane), 1.01 (d, J=12.4 Hz, 12H) ppm. ESI-MS m/z calc. 966.28, found 967.15 (M+1)⁺.

### Step 2: tert-Butyl (2-(5-((2R,8S)-2-(benzyloxy)-8-((tert-butyldiphenylsilyl)oxy)-1,1,1-trifluorononan-2-yl)-1,3,4-oxadiazol-2-yl)-6-bromo-5-(trifluoromethyl)pyridin-3-yl)carbamate (66)

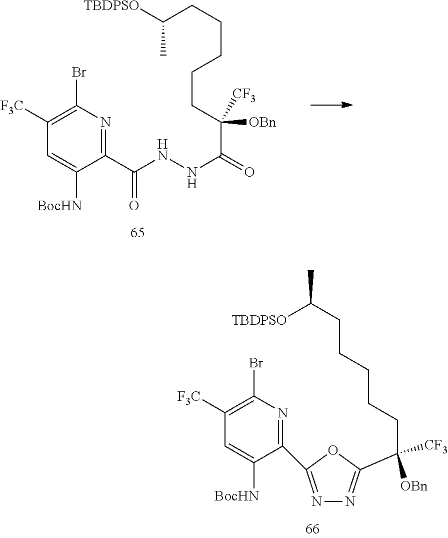

In a 500 mL RBF, tert-butyl (2-(2-((2R,8S)-2-(benzyloxy)-8-((tert-butyldiphenylsilyl)oxy)-2-(trifluoromethyl)nonanoyl)hydrazine-1-carbonyl)-6-bromo-5-(trifluoromethyl)pyridin-3-yl)carbamate (12.5 g, 12.914 mmol, 1 equiv.), N,N-diisopropylethylamine (5.007 g, 6.748 mL, 0.742 g/mL, 38.743 mmol, 3 equiv.) and 1,4-diazabicyclo[2.2.2]octane (144.863 mg, 1.291 mmol, 0.1 equiv.) in CH₃CN (100 mL, 8 Vols), at ambient added p-toluenesulfonyl chloride (3.311 g, 17.366 mmol, 1.345 equiv.). The mixture was stirred at ambient temperature After complete reaction by LC analysis, removed ACN by rotary evaporation. Added IPAc (10 vols) to the mixture, added 10% aq w/v KHSO₄ (125 mL), stirred vigorously for 30 min, repeat. pH of aqueous layer was ˜2. Added 0.5 M NaOH aq. (125 mL) to the organic layer, stirred vigorously for 1 hr and determined the pH of the aqueous layer was ˜13. The organic layer was washed with brine/water (1:1, 100 mL×2), dried over Na₂SO₄, filtered, concentrated, and exchange solvent with heptane. The crude was purified by column (220 g, HP silica) eluting with EtOAc/hexane (0-5%) for 20 min, clean fractions were collected, concentrated, heptane added, and concentrated further to afford 11.67 g (95%) of tert-butyl (2-(5-((2R,8S)-2-(benzyloxy)-8-((tert-butyldiphenylsilyl)oxy)-1,1,1-trifluorononan-2-yl)-1,3,4-oxadiazol-2-yl)-6-bromo-5-(trifluoromethyl)pyridin-3-yl)carbamate. ¹H NMR (400 MHz, DMSO) δ 10.14 (s, 1H), 9.10 (s, 1H), 7.63-7.56 (m, 4H), 7.53-7.30 (m, 11H), 4.72-4.58 (m, 2H), 3.80 (p, J=5.9 Hz, 1H), 2.44-2.21 (m, 2H), 1.51 (s, 9H), 1.48-1.33 (m, 4H), 1.33-1.16 (m, 4H, corrected for presence of residual heptane), 1.07-0.95 (m, 12H) ppm. ESI-MS m/z calc. 948.27, found 966.25 (M+18)⁺.

### Step 3: tert-Butyl (2-(5-((2R,8R)-2-(benzyloxy)-1,1,1-trifluoro-8-hydroxynonan-2-yl)-1,3,4-oxadiazol-2-yl)-6-bromo-5-(trifluoromethyl)pyridin-3-yl)carbamate (68)

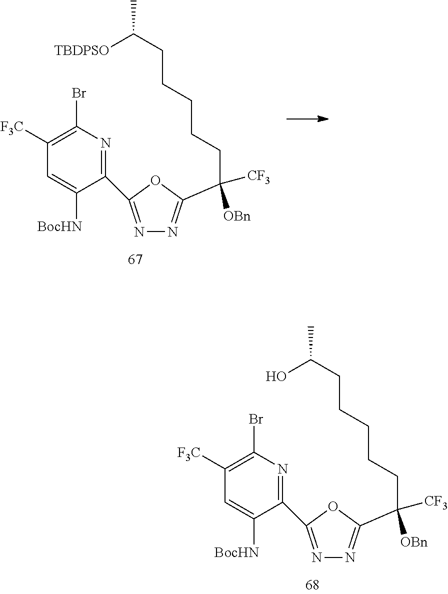

To a 250 mL RBF with tert-butyl N-(2-{5-[(2R,8R)-2-(benzyloxy)-8-[(tert-butyldiphenylsilyl)oxy]-1,1,1-trifluorononan-2-yl]-1,3,4-oxadiazol-2-yl}-6-bromo-5-(trifluoromethyl)pyridin-3-yl)carbamate (11.67 g, 12.285 mmol, 1 equiv.; is prepared in an analogous manner to the product of Example 27, Step 2) in THF (50 mL, 0.246 M, 4.284 Vols) was added tetrabutylammonium fluoride, 1.0 M solution in THF (4.818 g, 18.428 mL, 1 M, 18.428 mmol, 1.5 equiv.), the mixture was stirred at 40° C. After 41 hr, the mixture was concentrated by rotary evaporation. To the residue was added MTBE (10 vols). Added aq satd. NH₄Cl (60 mL), stirred for 30 min, phase separated, repeated, the organic layer was washed with brine/water (1:1, 60 mL). The organic layer was dried over Na₂SO₄, filtered, concentrated on rotary evaporator. The crude was purified by column (220 g, HP silica) chromatography eluting with EtOAc/hexane (0-20% EtOAc) over 25 min, the product eluted at 20% EtOAc/hexanes. Clean fractions were collected, concentrated, swap with heptane, dried under house vacuum at ambient, then dried under vacuum oven at 40° C. over 2 nights with nitrogen bleed affording the title compound as a viscous oil (4.2 g). ¹H NMR (400 MHz, DMSO) δ 10.13 (s, 1H), 9.10 (s, 1H), 7.51 (d, J=7.1 Hz, 2H), 7.47-7.31 (m, 3H), 4.75-4.60 (m, 2H), 4.30 (d, J=4.7 Hz, 1H), 3.60-3.51 (m, 1H), 2.48-2.31 (m, 2H), 1.53 (s, 11H), 1.43-1.21 (m, 6H, corrected for presence of residual heptane), 1.02 (d, J=6.1 Hz, 3H) ppm. ESI-MS m/z calc. 710.15, found 711.10 (M+1)⁺.

### Step 4: tert-Butyl ((4S,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate (70)

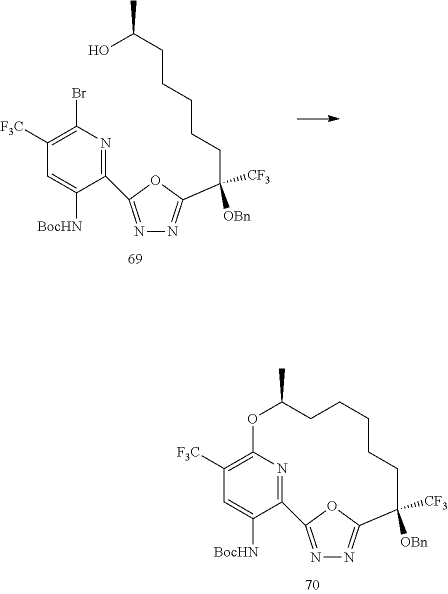 [SMILES: C[C@H](O)CCCCC[C@@](OCc1ccccc1)(c1nnc(-c2nc(Br)c(C(F)(F)F)cc2NC(=O)OC(C)(C)C)o1)C(F)(F)F ; C[C@H]1CCCCC[C@](OCc2ccccc2)(C(F)(F)F)c2nnc(o2)-c2nc(c(C(F)(F)F)cc2NC(=O)OC(C)(C)C)O1]

A room temperature solution of tert-butyl (2-(5-((2R,8R)-2-(benzyloxy)-1,1,1-trifluoro-8-hydroxynonan-2-yl)-1,3,4-oxadiazol-2-yl)-6-bromo-5-(trifluoromethyl)pyridin-3-yl)carbamate (101.0 mg; 0.142 mmoles; 1 eq is prepared in an analogous manner to the product of Example 27, Step 3) in toluene (2.8 mL; 0.05 M; 28 volumes) was treated with K₃PO₄ (93.1 mg; 0.439 mmoles; 3.09 eq). This mixture was sparged with argon before adding XantPhos G4 (14.1 mg; 0.0146 mmoles; 0.10 eq) and then sparged again with argon. After sealing the vial with a Teflon-lined cap, the reaction was heated at 100° C. for 23 hours. The reaction was cooled to room temperature and diluted with water (2 mL) and toluene (2 mL). Layers were separated, and the aq. phase was extracted with toluene (2 mL). The combined organic layers were washed with brine, dried (Na₂SO₄), filtered, and concentrated to afford 110.5 mg of crude material. The crude material was purified on 11 g of silica gel. The column was packed and loaded with 25% DCM/hexanes. The column was eluted with ˜4 column volumes (˜22 mL) of 25% DCM/hexanes and ˜3 column volumes of 1:1 DCM/hexanes. Fractions 12-17The product-containing fractions were combined, concentrated, and dried (high vacuum, room temperature, overnight). The title compound, tert-butyl ((4S,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate (R,S-diastereomer), was obtainedisolated as a white foam (53.6 mg, 59.9% yield).

tert-Butyl ((4S,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate (64):

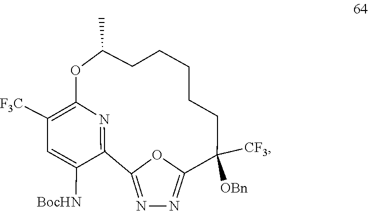 [SMILES: C[C@@H]1CCCCC[C@](OCc2ccccc2)(C(F)(F)F)C2=NN=C(O2)c2nc(c(C(F)(F)F)cc2NC(=O)OC(C)(C)C)O1]

is prepared in a manner analogous to the product of Example 27, Step 4 (tert-butyl ((4S,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate).

### Example 28: Alternative Synthesis of tert-Butyl ((4S,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate (64)

### Step 1: tert-Butyl (2-(2-((2R,8R)-2-(benzyloxy)-8-((tert-butyldiphenylsilyl)oxy)-2-(trifluoromethyl)nonanoyl)hydrazine-1-carbonyl)-6-(methylthio)-5-(trifluoromethyl)pyridin-3-yl)carbamate (71)

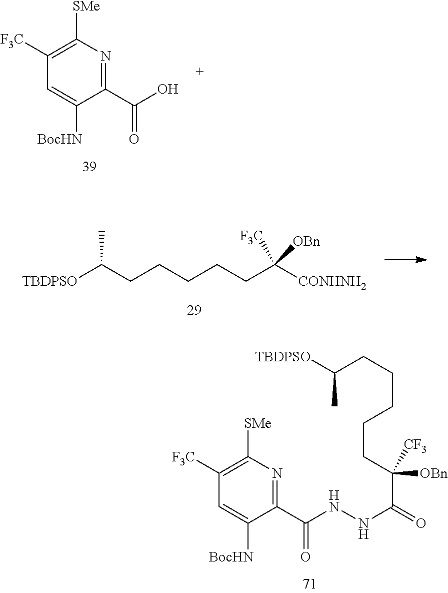

To 3-[(tert-butoxycarbonyl)amino]-6-(methylsulfanyl)-5-(trifluoromethyl)pyridine-2-carboxylic acid (2.623 g, 7.445 mmol, 1 equiv.) and (2R,8R)-2-(benzyloxy)-8-[(tert-butyldiphenylsilyl)oxy]-2-(trifluoromethyl)nonanehydrazide (4.92 g, 8.189 mmol, 1.1 equiv.) in ethyl acetate (26.23 mL, 0.284 M, 10 Vols), was added 4-methylmorpholine (1.506 g, 1.637 mL, 0.92 g/mL, 14.889 mmol, 2 equiv.). After stirring, 1-propanephosphonic acid cyclic anhydride (T3P, 5.211 g, 4.875 mL, 1.069 g/mL, 8.189 mmol, 1.1 equiv.) was added. The mixture was stirred at ambient temperature After overnight, the reaction went to completion. Added 26 mL of water, stirred and phase split, added 26 mL 10% KHSO₄ (w/v) to the organic layer, stirred and phase split. The organic layer was washed with 26 mL brine/water (1:1), dried over Na₂SO₄, filtered, and concentrated on rotary evaporator. The crude mixture was purified by silica gel column chromatography (220 g HP silica) eluting with EtOAc/Hexanes (0-5% EtOAc) over 20 min. The product-containing fractions were collected, concentrated, residue was diluted with heptane, and then dried under house vacuum overnight affording the title compound as a viscous oil (6.678 g, yield 93%). ¹H NMR (400 MHz, DMSO) δ 10.73-10.61 (m, 2H), 10.31 (s, 1H), 9.03 (s, 1H), 7.64-7.57 (m, 4H), 7.51-7.29 (m, 11H), 4.81 (t, J=8.8 Hz, 2H), 3.82 (q, J=5.9 Hz, 1H), 2.71 (s, 3H), 2.21-1.97 (m, 2H), 1.45 (s, 13H), 1.25 (s, 4H, corrected for presence of residual heptane), 1.01 (d, J=12.9 Hz, 12H) ppm. ESI-MS m/z calc. 934.36, found 935.38 (M+1)⁺.

### Step 2: tert-Butyl (2-(5-((2R,9R)-2-(benzyloxy)-9-((tert-butyldiphenylsilyl)oxy)-1,1,1-trifluorodecan-2-yl)-1,3,4-oxadiazol-2-yl)-6-(methylthio)-5-(trifluoromethyl)pyridin-3-yl)carbamate (72)

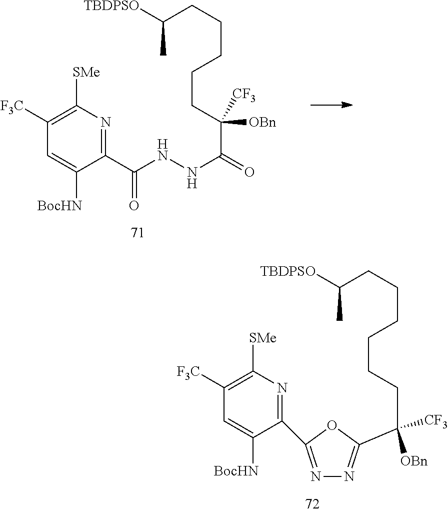

In a 100 mL RBF, tert-butyl N-(2-{[(2R,8R)-2-(benzyloxy)-8-[(tert-butyldiphenylsilyl)oxy]-2-(trifluoromethyl)nonanehydrazido]carbonyl}-6-(methylsulfanyl)-5-(trifluoromethyl)pyridin-3-yl)carbamate (2.956 g, 3.161 mmol, 1 equiv.), N,N-diisopropylethylamine (1225.662 mg, 1.652 mL, 0.742 g/mL, 9.483 mmol, 3 equiv.) and 1,4-diazabicyclo[2.2.2]octane (35.459 mg, 0.316 mmol, 0.1 equiv.) in CH₃CN (23.648 mL, 0.134 M, 8 Vols), at ambient temperature p-toluenesulfonyl chloride (810.397 mg, 4.251 mmol, 1.345 equiv.) was added. The reaction was stirred at ambient temperature After 1.5 h, the reaction went to completion by LC analysis. Removed ACN by rotary evaporator. Added IPAc (10 vols) to the mixture, added 10% KHSO₄ aq. (30 mL), stirred vigorously for 30 min, repeated until the pH of the aqueous layer was ˜2. Added 0.5 M NaOH aq. (30 mL) to the organic layer, stirred vigorously for 1 hr, at which point the pH of aqueous layer was ˜10. The organic layer was washed with brine/water (1:1.15 mL×2), dried over Na₂SO₄, filtered, concentrated, swapped solvent with heptane. The crude was purified by column (80 g, HP silica) eluting with EtOAc/hexanes (0-5% EtOAc) for 20 min, clean fractions were collected, concentrated, swapped with heptane affording 3.047 g of product as a viscous oil. ¹H NMR (400 MHz, DMSO) δ 9.89 (s, 1H), 8.92 (s, 1H), 7.63-7.54 (m, 4H), 7.47-7.27 (m, 11H), 4.75-4.58 (m, 2H), 3.80 (q, J=5.9 Hz, 1H), 2.57 (s, 3H), 2.44-2.21 (m, 2H), 1.50 (s, 17H, corrected for presence of residual heptane), 0.99 (d, J=16.6 Hz, 12H) ppm. ESI-MS m z calc. 916.35, found 917.37 (M+1)⁺.

### Step 3: tert-Butyl (2-(5-((2R,9R)-2-(benzyloxy)-9-((tert-butyldiphenylsilyl)oxy)-1,1,1-trifluorodecan-2-yl)-1,3,4-oxadiazol-2-yl)-6-(methylsulfonyl)-5-(trifluoromethyl)pyridin-3-yl)carbamate (73)

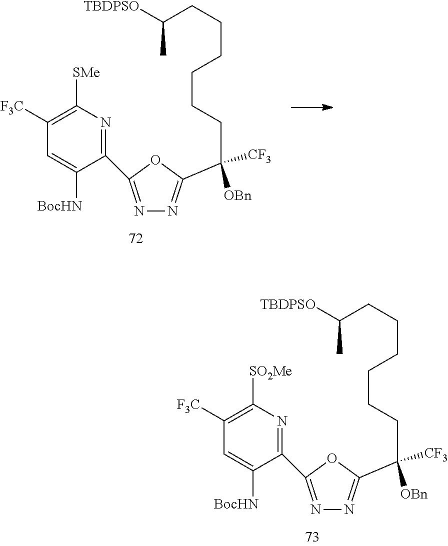

A mixture of tert-butyl N-(2-{5-[(2R,8R)-2-(benzyloxy)-8-[(tert-butyldiphenylsilyl)oxy]-1,1,1-trifluorononan-2-yl]-1,3,4-oxadiazol-2-yl}-6-(methylsulfanyl)-5-(trifluoromethyl)pyridin-3-yl)carbamate (2.834 g, 2.936 mmol, 1 equiv.) in CH₃CN (45.344 mL, 0.065 M, 16 Vols) and water (21.255 mL, 0.138 M, 7.5 Vols) was added sodium periodate (1.884 g, 8.807 mmol, 3 equiv.) followed by ruthenium(III) chloride hydrate (13.237 mg, 0.059 mmol, 0.02 equiv.). The reaction mixture was stirred at ambient temperature After 1 h, the reaction went to completion by LC analysis. Reaction was stopped, the mixture was filtered, the filtrate was diluted with 10 mL of water, and extracted with 30 mL of EtOAc. The organic layer was dried over sodium sulfate, filtered, and concentrated avia rotary evaporaion. The crude was purified by column chromatography (80 g, HP silica) eluting with EtOAc/Hexanes (0-10% EtOAc) over 20 min. Clean Product-containing fractions were collected, concentrated, diluted with heptane and dried under house vacuum overnight affording the title compound as a white solid (2.534 g, 90% yield). ¹H NMR (400 MHz, DMSO) δ 10.30 (s, 1H), 9.27 (s, 1H), 7.60 (d, J=6.7 Hz, 4H), 7.50-7.26 (m, 11H), 4.75-4.56 (m, 2H), 3.81 (q, J=5.9 Hz, 1H), 3.44 (s, 3H), 2.44-2.24 (m, 2H), 1.53 (s, 9H), 1.49-1.32 (m, 4H), 1.24 (s, 4H), 0.98 (s, 12H) ppm. ESI-MS m/z calc. 948.34, found 949.34 (M+1)⁺.

### Step 4: tert-Butyl (2-(5-((2R,9R)-2-(benzyloxy)-1,1,1-trifluoro-9-hydroxydecan-2-yl)-1,3,4-oxadiazol-2-yl)-6-(methylsulfonyl)-5-(trifluoromethyl)pyridin-3-yl)carbamate (74)

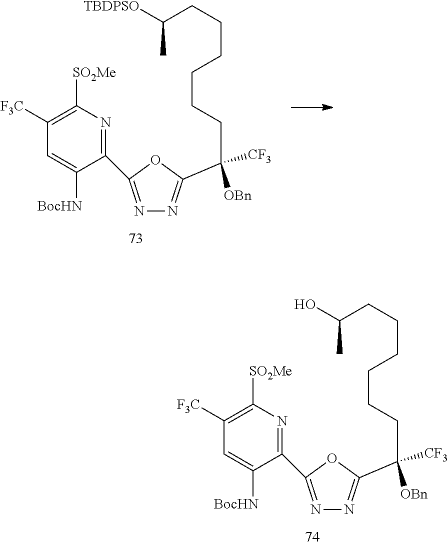

To a 40 mL vial containing tert-butyl N-(2-{5-[(2R,8R)-2-(benzyloxy)-8-[(tert-butyldiphenylsilyl)oxy]-1,1,1-trifluorononan-2-yl]-1,3,4-oxadiazol-2-yl}-6-methanesulfonyl-5-(trifluoromethyl)pyridin-3-yl)carbamate (1.9 g, 2.002 mmol, 1 equiv.) in THF (7.6 mL, 0.263 M, 4 Vols) was added tetrabutylammonium fluoride (1.309 g, 5.005 mL, 1 M in THF, 5.005 mmol, 2.5 equiv.) The mixture was stirred at 40° C. for approximately 3 days, after which the solvent was removed by rotary evaporation. To the residue was added MTBE (10 vols). Added aq. saturated NH₄Cl (10 mL), stirred for 30 min, isolated the aqueous layer, and repeated. The organic layer was washed with brine/water (1:1, 10 mL), dried over Na₂SO₄, filtered, and concentrated. The crude was purified by column (80 g, HP silica) eluting with EtOAc/hexanes (0-40% EtOAc) for 20 min, product eluted at 40% EtOAc/hexanes. Clean fractions were collected, concentrated, and triturated with heptane. The obtained solid was collected by filtration, dried under vacuum oven at 40° C. overnight with a nitrogen bleed affording the title compound as a white solid (0.96 g). ¹H NMR (400 MHz, DMSO) δ 10.30 (s, 1H), 9.28 (s, 1H), 7.46-7.27 (m, 5H), 4.75-4.62 (m, 2H), 4.30 (d, J=4.7 Hz, 1H), 3.59-3.50 (m, 1H), 3.46 (s, 3H), 2.47-2.30 (m, 2H), 1.55 (s, 11H), 1.43-1.22 (m, 6H, corrected for presence of residual heptane), 1.02 (d, J=6.1 Hz, 3H) ppm. ESI-MS m/z calc. 710.22, found 711.30 (M+1)⁺.

### Step 5: tert-Butyl ((4R,10R)-10-(benzyloxy)-4-methyl-2⁵,10-bis(trifluoromethyl)-3-oxa-1(2,5)-oxadiazola-2(2,6)-pyridinacyclodecaphane-23-yl)carbamate (64)

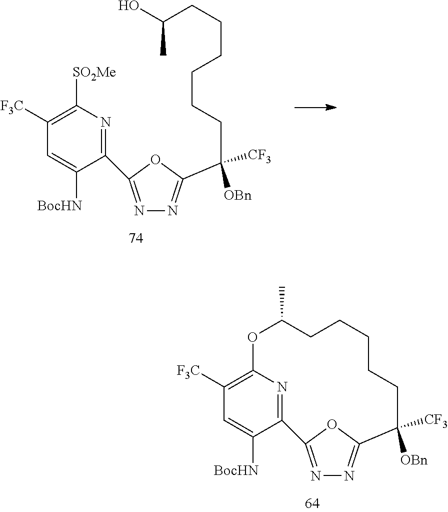 [SMILES: C[C@@H](O)CCCCCC[C@@](OCc1ccccc1)(c1nnc(-c2nc(S(C)(=O)=O)c(C(F)(F)F)cc2NC(=O)OC(C)(C)C)o1)C(F)(F)F ; C[C@@H]1CCCCC[C@](OCc2ccccc2)(C(F)(F)F)c2nnc(o2)-c2nc(c(C(F)(F)F)cc2NC(=O)OC(C)(C)C)O1]

In a 7 mL clear vial, to the tert-butyl J-(2-{5-[(2R,8R)-2-(benzyloxy)-1,1,1-trifluoro-8-hydroxynonan-2-yl]-1,3,4-oxadiazol-2-yl}-6-methanesulfonyl-5-(trifluoromethyl) pyridin-3-yl) carbamate (100 mg, 0.136 mmol, 1 equiv.) in DMF (1 mL, 0.136 M, 10 Vols) was added 2-methylpropan-2-olate (0.018 g, 0.164 mL, 1 M, 0.164 mmol, 1.2 equiv.) slowly with cooling by a water bath. The light-yellow reaction solution became red. The addition was done after 1 min, the mixture was stirred at ambient temperature After 30 min, 15% conversion by LC analysis. The conversion did not change after 2 hr. Added 2-methylpropan-2-olate (0.018 g, 0.164 mL, 1 M, 0.164 mmol, 1.2 equiv.) slowly (1 min) to the mixture under. Continued stirring at ambient temperature overnight. The resulting mixture was diluted with EtOAc and water. The organic layer was isolated, washed with brine, dried over Na₂SO₄, filtered, concentrated. The crude residue was purified by column chromatography (24 g, HP silica) eluting with EtOAc/hexanes (0-10% EtOAc). Product-containing fractions were collected, concentrated, diluted solvent with heptane, and dried to afford the title compound. ¹H NMR (400 MHz, DMSO) δ 9.23 (s, 1H), 8.77 (s, 1H), 7.39-7.28 (m, 5H), 4.96-4.86 (m, 1H), 4.72-4.51 (m, 2H), 2.62 (t, J=11.9 Hz, 1H), 2.29-2.16 (m, 1H), 1.80 (s, 1H), 1.65 (d, J=8.3 Hz, 2H), 1.50 (s, 12H), 1.40 (d, J=6.4 Hz, 3H, corrected for heptane), 1.25 (d, J=13.7 Hz, 2H) ppm. ESI-MS m/z calc. 630.23, found 631.20 (M+1)⁺.

### Example 29: Alternative Synthesis of tert-Butyl ((4S,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate (70)

### Step 1: Hept-3-yn-2-ol (8)

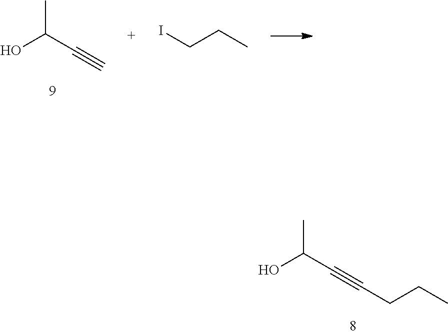

To a solution of but-3-yn-2-ol (50 g, 713.37 mmol, 55.93 mL, 1 eq) in THF (1000 mL) and HMPA (250 mL) was added dropwise n-BuLi (2.5 M, 570.70 mL, 2 eq) at −78° C. over 2 h under N₂. Then the mixture was stirred at −78° C. for 0.5 h and warmed to −20° C. Then 1-iodopropane (115.20 g, 677.70 mmol, 66.21 mL, 0.95 eq) was added dropwise to the mixture at −20° C. over 0.5 hr. The reaction was stirred at 25° C. for 1 h. TLC (PE:EA=10:1, Rf=0.33) showed new spots with larger polarity was detected. The reaction mixture was poured into ammonium chloride saturated solution (1500 mL). The organic phase was separated, the aqueous phase was extracted with DCM (1000 mL×2). The organic phase was washed with brine (2000 mL), dried with anhydrous Na₂SO₄, filtered and concentrated in vacuum at 30° C. The residue was purified by column chromatography (SiO2, Petroleum ether/Ethyl acetate=I/O to 100/1). Then the hept-3-yn-2-ol (63 g, 561.65 mmol, 39.37% yield) was obtained as yellow liquid. ¹H NMR of Compound 8: (400 MHz, chloroform-d) δ ppm 0.97 (t, J=7.6 Hz, 3H), 1.42 (d, J=6.4 Hz, 3H), 1.47-1.58 (m, 2H), 1.90 (s, 1H), 2.15-2.19 (m, 2H), 4.49-4.54 (m, 1H) ppm.

### Step 2: Hept-3-yn-2-one

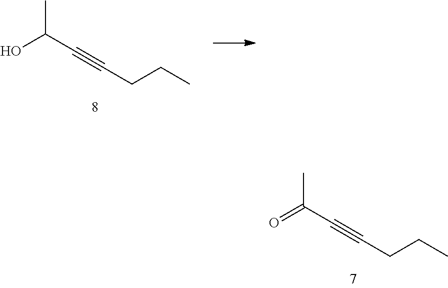

To a solution of hept-3-yn-2-ol (150 g, 1.34 mol, 1 eq) in DCM (1500 mL) was added MnO₂ (813.80 g, 9.36 mol, 7 eq) in portions at 25° C. under N₂. The reaction was stirred at 40° C. for 24 h. TLC (PE:EA=10:1, Rf=0.47) showed new spot. The reaction was filtered through a pad of Celite. The cake was washed with DCM (500×5 mL). The organic phase was concentrated in vacuum at 30° C. The product was used directly without further purification. Then hept-3-yn-2-one (125 g, 1.13 mol, 84.86% yield) was obtained as yellow liquid. ¹H NMR of Compound 7: (400 MHz, chloroform-d) 6 ppm 0.95 (t, J=7.2 Hz, 3H), 1.52-1.57 (m, 2H), 2.25-2.29 (m, 5H).

### Step 3: (R)-hept-3-yn-2-ol (75)

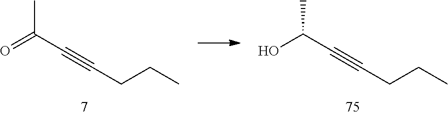

A solution of N-[(1R,2R)-2-amino-1,2-diphenyl-ethyl]-4-methyl-benzenesulfonamide (6.65 g, 18.16 mmol, 0.04 eq) and dichlororuthenium; 1-isopropyl-4-methyl-benzene (5.56 g, 9.08 mmol, 0.02 eq) in water (125 mL) was degassed 3 times and purged with N₂ 3 times. The mixture was heated to 80° C. and stirred for 1 h. Then the mixture was cooled to 25° C. A solution of hexadecyl(trimethyl)ammonium; bromide (16.54 g, 45.39 mmol, 0.1 eq), sodium formate (250.04 g, 3.68 mol, 198.44 mL, 8.1 eq) and hept-3-yn-2-one (50 g, 453.91 mmol, 1 eq) in H₂O (1725 mL) was added to the reaction mixture. The mixture was stirred at 25° C. for 16 h under N₂. TLC (PE:EA=10:1, Rf=0.21) showed one new spot. The reaction mixture was filtered through a pad of Celite and the cake was washed with MTBE (200 mL×3). Water (1500 mL) was added to the mother liquor and the organic phase was separated. The aqueous phase was extracted with MTBE (700 mL×4). The combined organic phase was dried over Na₂SO₄, filtered, and concentrated under reduced pressure at 30° C. The crude product was purified by column chromatography (SiO₂, Petroleum ether/Ethyl acetate=I/O to 25/1). (2R)-Hept-3-yn-2-ol (65 g, 579.48 mmol, 63.83% yield) was obtained as a brown liquid. ¹H NMR of Compound 75: (400 MHz, CHLOROFORM-d) δ 0.98 (t, J=7.2 Hz, 3H), 1.42-1.44 (d, J=6.80 Hz, 3H), 1.47-1.57 (m, 2H), 2.16-2.20 (m, 2H), 4.49-4.54 (m, 1H) ppm.

### Step 4: (R)-Hept-6-yn-2-ol (76)

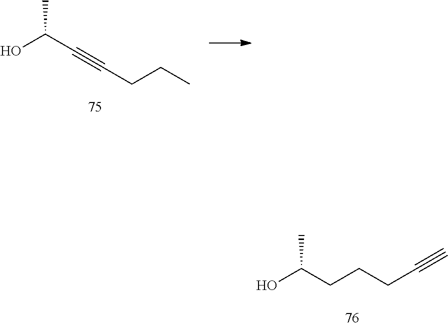

Lithium metal (24.74 g, 3.56 mol, 30.17 mL, 7.27 eq) was added to dry 1,3-diaminopropane (860 mL) in portions at 75° C. over 3 h and the mixture was stirred at 75° C. for 16 h. Then the mixture was cooled to 25° C. and t-BuOK (210.18 g, 1.87 mol, 3.82 eq) was added into the mixture in portions and stirred at 25° C. for 30 min. Then (2R)-hept-3-yn-2-ol (55 g, 490.33 mmol, 1 eq) was added to the reaction mixture and stirred at 25° C. for 3 h under N₂. TLC (PE:EA=3:1, Rf=0.36) showed anew spot. The reaction was quenched by ice water (4000 mL), follow up extracted with DCM (500 mL×9). The combined organic phases were dried over Na₂SO₄, filtered, and concentrated under reduced pressure at 30° C. to give a residue. The crude product was purified by column chromatography (SiO₂, Petroleum ether/Ethyl acetate=100/0 to 10/100). 45 g of (2R)-hept-6-yn-2-ol (45 g, 393.16 mmol, 29% yield) was obtained with 98% purity as yellow liquid. An additional 32 g of (2R)-hept-6-yn-2-ol (32 g, 268.17 mmol, 21% yield) was obtained with 94% purity as a brown liquid. ¹H NMR of Compound 76 ((R)-hept-6-yn-2-ol): ¹H NMR (400 MHz, chloroform-d), δ 1.21 (d, J=8.0 Hz, 3H), 1.55-1.73 (m, 4H), 1.96-1.97 (t, J=4.0 Hz, 1H), 2.21-2.26 (m, 2H), 3.82-3.86 (m, 1H) ppm; MS of Compound 76 ((R)-hept-6-yn-2-ol): m/z=95[M−OH]⁺.

### Step 5: (R)-Hept-6-yn-2-yl benzoate (77)

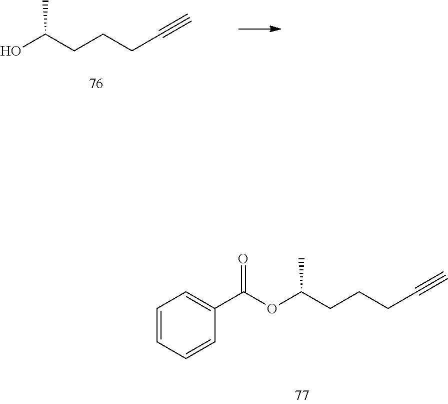

To a solution of compound 76 ((R)-hept-6-yn-2-ol) (22.5 g, 200.59 mmol, 1 eq) in DCM (450 mL) was added TEA (36.54 g, 361.06 mmol, 50.26 mL, 1.8 eq), DMAP (12.25 g, 100.30 mmol, 0.5 eq) and benzoyl chloride (39.47 g, 280.83 mmol, 32.62 mL, 1.4 eq) at 25° C. The reaction mixture was stirred at 25° C. for 3 hr. TLC (PE:EA=10:1) showed compound 76 was consumed completely and one new spot formed. The reaction mixture was quenched by adding water (2000 mL) and then extracted with DCM (500 mL×3). The combined organic layers were dried over Na₂SO₄, filtered, and concentrated under reduced pressure to give a residue. The residue was purified by column chromatography (SiO₂, Petroleum ether/Ethyl acetate=0 to 10/1) to obtain compound 77 (2 batches; 82 g, 379.15 mmol, 94.51% yield) as a colourless oil. LCMS of Compound 77: Rt=0.949 min, purity: 97.39%, m/z: 217.2 [M+H]⁺; ¹H NMR of Compound 77: (CDCl₃, 400 MHz): δ 8.07-8.03 (m, 2H), 7.56-7.53 (m, 1H), 7.46-7.42 (m, 2H), 5.22-5.17 (m, 1H), 2.27-2.22 (m, 2H), 1.97 (t, J=2.63 Hz, 1H), 1.87-1.60 (m, 4H), 1.37 (d, J=6.25 Hz, 3H) ppm.

### Step 6: (2R,8S)-8-(Ethoxycarbonyl)-9,9,9-trifluoro-8-hydroxynon-6-yn-2-yl benzoate (79)

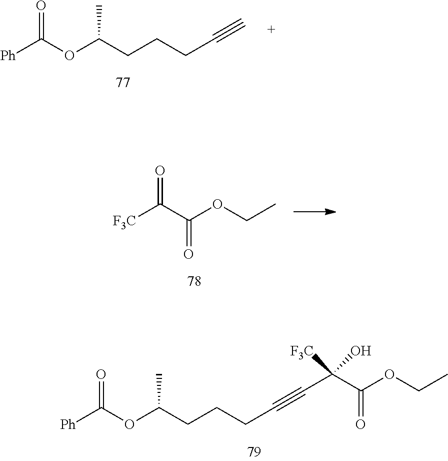 [SMILES: C#CCCC[C@@H](C)OC(=O)c1ccccc1 ; CCOC(=O)C(=O)C(F)(F)F ; CCOC(=O)[C@@](O)(C#CCCC[C@@H](C)OC(=O)c1ccccc1)C(F)(F)F]

To a solution of compound 77 ((R)-hept-6-yn-2-yl benzoate) (36 g, 166.45 mmol, 1.2 eq) and compound 78 (23.59 g, 138.71 mmol, 18.43 mL, 1 eq) in Et₂O (216 mL) was added catalyst [2-[(3aR,8bS)-4,8b-dihydro-3aH-indeno[1,2-d]oxazol-2-yl]-6-[(4S)-4-isopropyl-4,5-dihydrooxazol-2-yl]-4-nitro-phenyl]-diacetoxy-hydroxy-rhodium (4.37 g, 6.94 mmol, 0.05 eq) under N₂ atmosphere. The reaction was stirred at 25° C. for 24 h under N₂ atmosphere. TLC (PE:EA=5:1) showed a new spot was detected and HPLC showed 35.1% of compound 77 was remained and 54.8% of compound 79 was detected. The reaction mixture was concentrated under reduced pressure at 30° C. to give a residue. The residue was purified by column chromatography (SiO₂, Petroleum ether/Ethyl acetate=100/1 to 10/1) to obtained compound 79 ((2R,8S)-8-(Ethoxycarbonyl)-9,9,9-trifluoro-8-hydroxynon-6-yn-2-yl benzoate) (69.7 g, 179.32 mmol, 64.64% yield, 99.4% purity) as a light yellow oil and compound 77 (25 g) was recycled as a colorless oil. LCMS of Compound 77 ((2R,8S)-8-(Ethoxycarbonyl)-9,9,9-trifluoro-8-hydroxynon-6-yn-2-yl benzoate): Rt=0.994 min, purity: 100%, m/z: 409.1 [M+Na]⁺; SFC of Compound 77 ((2R,8S)-8-(Ethoxycarbonyl)-9,9,9-trifluoro-8-hydroxynon-6-yn-2-yl benzoate): Rt=0.745 min, 92.16% ee; ¹H NMR of Compound 77 ((2R,8S)-8-(Ethoxycarbonyl)-9,9,9-trifluoro-8-hydroxynon-6-yn-2-yl benzoate): (DMSO-d₆, 400 MHz): δ 8.00-7.89 (m, 3H), 7.68-7.63 (m, 1H), 7.55-7.49 (m, 2H), 5.07 (m, 1H) 4.30-4.17 (m, 2H), 2.34 (t, J=6.8 Hz, 2H), 1.84-1.67 (m, 2H), 1.63-1.48 (m, 2H), 1.29 (d, J=6.4 Hz, 3H), 1.20 (t, J=7.2 Hz, 3H) ppm.

### Step 7: (2R,8R)-2,8-Dihydroxy-2-(trifluoromethyl)non-3-ynoic acid (81)

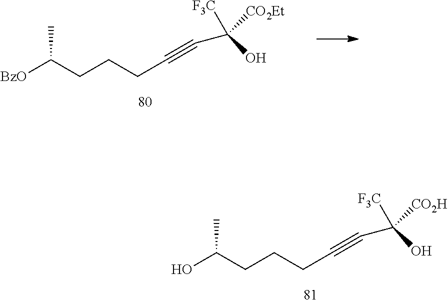

A solution of ethyl (2R,8R)-8-(benzoyloxy)-2-hydroxy-2-(trifluoromethyl)non-3-ynoate (96.5 g, 250 mmol, 1 equiv.) in methanol (772 mL, 8 Vols) was stirred at RT when 6 M NaOH (125 mL, 749 mmol, 3 equiv.) was added over 1 min. The temperature of the mixture increased from 18 to 30° C. during the addition and was increased further to 50° C. with external heating for ˜1 h when UPLC-MS indicated the reaction was completed. The mixture was cooled to RT and concentrated (40° C./40 torr) to remove most of the MeOH. The concentrated was partitioned between MTBE (772 mL, 8 Vols) and 2 M HCl (500 mL, 999 mmol, 4 equiv.) to pH 1. The phases were separated; the organic phase was washed with water (386 mL, 4 Vols, 2×), dried (Na₂SO₄), and concentrated (40° C./300-20 torr) to afford 123.2 g (126%) of a white pasty solid that was used as is for the next step.

### Step 8: Benzyl (2R,8R)-2,8-dihydroxy-2-(trifluoromethyl)non-3-ynoate

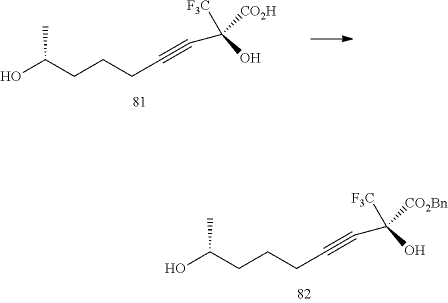 [SMILES: C[C@@H](O)CCCC#C[C@@](O)(C(=O)O)C(F)(F)F ; C[C@@H](O)CCCC#C[C@@](O)(C(=O)OCc1ccccc1)C(F)(F)F]

A suspension of the alkyne (63.5 g, 250 mmol, 1 equiv.)/benzoic acid (30.5 g, 250 mmol, 1 equiv.) mixture and NaHCO₃ (46.2 g, 550 mmol, 2.2 equiv.) in DMF (444 mL, 7 Vols) was stirred at RT then BnBr (85.4 g, 59.4 mL, 500 mmol, 2 equiv.) was added over 1 min. A slight endotherm (23.5 to 23° C.) was observed. The mixture was warmed at 60° C. for ˜2 h when UPLC-MS showed the reaction was completed. The suspension was cooled to RT and partitioned between MTBE (825 mL, 13 Vols) and water (762 mL, 12 Vols). The phases were separated, and the organic phase was washed with water (508 mL, 8 Vols, 3×), dried (Na₂SO₄), and concentrated (40° C./350-20 torr) to afford the expected benzyl esters (115.3 g; 83% combined product yield) as a yellow-tinted liquid. The crude product was split in two portions (20 g and 95.3 g) and purified via silica gel chromatography (330-g SiO₂ cartridge and 750-g SiO2 cartridge respectively) equilibrated with 10% EtOAc/hexanes and eluted with a gradient from 10-50% EtOAc. The crude material was loaded onto the columns as a DCM solution. The pure fractions were combined and concentrated to afford pure (R,R)-alkyne ester (70.4 g; 82%) as a colorless oil.

### Step 9: Methyl 6-(((2S,8R)-8-((benzyloxy)carbonyl)-9,9,9-trifluoro-8-hydroxynon-6-yn-2-yl)oxy)-3-nitro-5-(trifluoromethyl)picolinate (83)

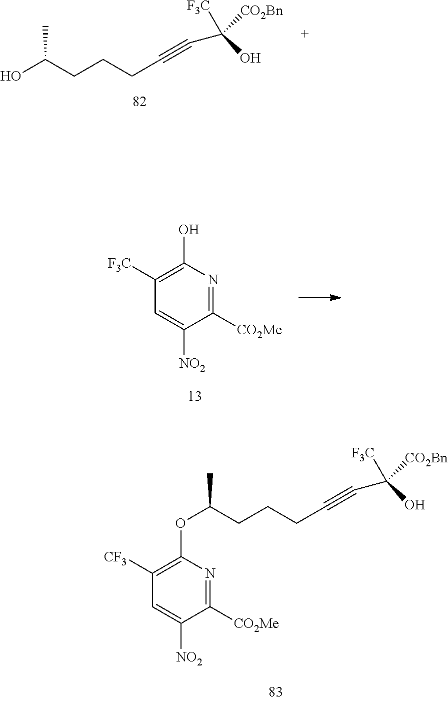 [SMILES: C[C@@H](O)CCCC#C[C@@](O)(C(=O)OCc1ccccc1)C(F)(F)F ; COC(=O)c1nc(O)c(C(F)(F)F)cc1[N+](=O)[O-] ; COC(=O)c1nc(O[C@@H](C)CCCC#C[C@@](O)(C(=O)OCc2ccccc2)C(F)(F)F)c(C(F)(F)F)cc1[N+](=O)[O-]]

A suspension of methyl 6-hydroxy-3-nitro-5-(trifluoromethyl)pyridine-2-carboxylate (35.0 g, 131 mmol, 1 equiv.), benzyl (2R,8R)-2,8-dihydroxy-2-(trifluoromethyl)non-3-ynoate (49.8 g, 145 mmol, 1.1 equiv.), and Ph₃P (41.4 g, 158 mmol, 1.2 equiv.) in PhMe (350 mL, 10 Vols) was stirred at RT. A solution of DIAD (31.9 g, 158 mmol, 1.2 equiv.) in PhMe (30 mL) was added over 20 min while keeping the reaction temperature below 25° C. Note: The addition is exothermic; the temperature increased from 15 to 22° C. when external cooling was used to maintain the temperature at −22° C. After the addition was completed, the orange reaction solution was stirred at RT for ˜1 h when UPLC-MS showed the reaction was nearly completed. The reaction solution was diluted with heptane (350 mL, 10 Vols) in an attempt to precipitate the Ph₃PO but, in this case, no filterable solid formed; however, two phases formed with the lower phase being primarily Ph₃PO. The upper phase was decanted and the lower phase was extracted with 1:1 PhMe:heptane (2×250-mL) and then 2:1 PhMe:heptane (2×250-mL). Combined the extracts with the upper phase and concentrated to afford crude product (138 g, 177% theory) as a yellow, viscous oil. The crude product was purified via silica gel chromatography (750-g SiO2 cartridge) equilibrated with 10% EtOAc/hexanes and eluted with a gradient from 10-60% EtOAc. The crude material was loaded onto the columns as a DCM solution. The pure fractions were combined and concentrated to afford pure methyl 6-(((2S,8R)-8-((benzyloxy)carbonyl)-9,9,9-trifluoro-8-hydroxynon-6-yn-2-yl)oxy)-3-nitro-5-(trifluoromethyl)picolinate (39.7 g; 51%) as a colorless oil. UPLC-MS: t_{R}=2.12 min/M+1=593.

### Step 10: (2R,8S)-8-((5-Amino-6-(methoxycarbonyl)-3-(trifluoromethyl)pyridin-2-yl)oxy)-2-hydroxy-2-(trifluoromethyl)nonanoic acid (84)

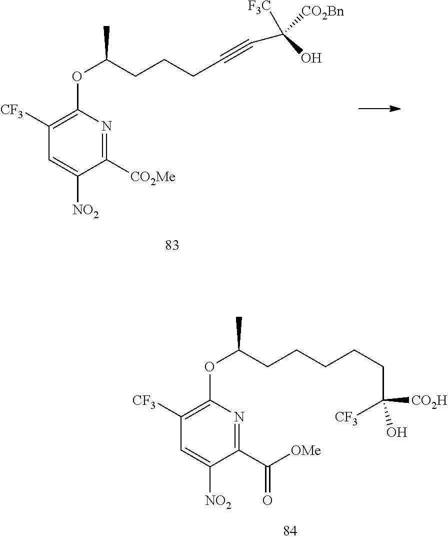 [SMILES: COC(=O)c1nc(O[C@@H](C)CCCC#C[C@@](O)(C(=O)OCc2ccccc2)C(F)(F)F)c(C(F)(F)F)cc1[N+](=O)[O-] ; COC(=O)c1nc(O[C@@H](C)CCCCC[C@@](O)(C(=O)O)C(F)(F)F)c(C(F)(F)F)cc1[N+](=O)[O-]]

A suspension of methyl 6-{[(2S,8R)-9-(benzyloxy)-8-hydroxy-9-oxo-8-(trifluoromethyl)non-6-yn-2-yl]oxy}-3-nitro-5-(trifluoromethyl)pyridine-2-carboxylate (9.5 g, 16 mmol, 1 equiv.) and 10 wt % Pd on carbon (1.7 g) in EtOAc (76 mL, 8 Vols) was evacuated/backfilled with N₂ (3×). The suspension was then evacuated and backfilled with H₂ (balloon). After 17 h, UPLC-MS showed reaction completion. The catalyst was removed by filtration (Celite pad) and the filter-bed was rinsed with EtOAc (3×20-mL). The combined filtrate and washings were concentrated (40° C./15 torr) to afford (2R,8S)-8-((5-amino-6-(methoxycarbonyl)-3-(trifluoromethyl)pyridin-2-yl)oxy)-2-hydroxy-2-(trifluoromethyl)nonanoic acid (8.13 g) as a yellow syrup (contains some residual solvent). UPLC-MS: single peak at t_{R}=1.73 min/M+1=477.

### Step 11: (2R,8S)-8-((5-((tert-Butoxycarbonyl)amino)-6-(methoxycarbonyl)-3-(trifluoromethyl)pyridin-2-yl)oxy)-2-hydroxy-2-(trifluoromethyl)nonanoic acid (88)

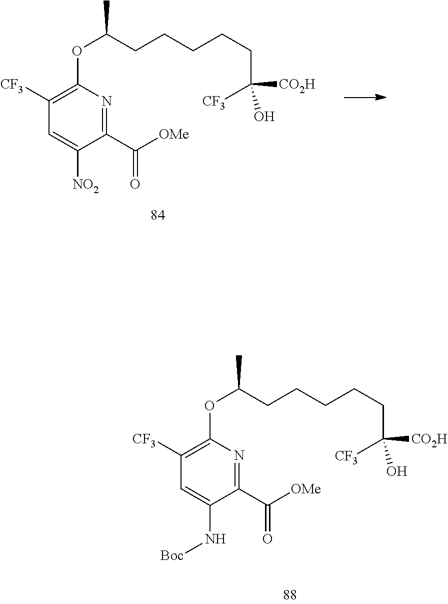 [SMILES: COC(=O)c1nc(O[C@@H](C)CCCCC[C@@](O)(C(=O)O)C(F)(F)F)c(C(F)(F)F)cc1[N+](=O)[O-] ; COC(=O)c1nc(O[C@@H](C)CCCCC[C@@](O)(C(=O)O)C(F)(F)F)c(C(F)(F)F)cc1NC(=O)OC(C)(C)C]

A solution of (2R,8S)-8-{[5-amino-6-(methoxycarbonyl)-3-(trifluoromethyl)pyridin-2-yl]oxy}-2-hydroxy-2-(trifluoromethyl)nonanoic acid (4.50 g, 9.44 mmol, 1 equiv.) and NMM (1.91 g, 2.08 mL, 18.9 mmol, 2 equiv.) in DCM (36 mL, 8 Vols) was stirred at RT when Boc₂O (5.15 g, 23.6 mmol, 2.5 equiv.) and DMAP (0.23 g, 1.9 mmol, 0.2 equiv.) were added. After 15 h, UPLC-MS showed no starting material remained. The mixture was concentrated to remove most of the DCM and then partitioned between EtOAc (50 mL) and 1 M HCl (24 mL, 1 M, 2.5 equiv.). The phases were separated (aqueous pH 1), the organic layer washed with water (25 mL), dried (Na₂SO₄), and concentrated to afford crude (2R,8S)-8-((5-((tert-butoxycarbonyl)amino)-6-(methoxycarbonyl)-3-(trifluoromethyl)pyridin-2-yl)oxy)-2-hydroxy-2-(trifluoromethyl)nonanoic acid (6.0 g; ˜105% theory) as an orange oil. The crude product was purified via silica gel chromatography (120-g SiO₂ cartridge) equilibrated with 10% EtOAc/hexanes and eluted with a gradient from 10-40% EtOAc. The crude material was loaded onto the columns as a DCM solution. Later eluting fractions were combined and concentrated to afford the title compound (2.52 g).

### Step 12: (2R,8S)-8-((5-((tert-Butoxycarbonyl)amino)-6-(hydrazinecarbonyl)-3-(trifluoromethyl)pyridin-2-yl)oxy)-2-hydroxy-2-(trifluoromethyl)nonanoic acid (85)

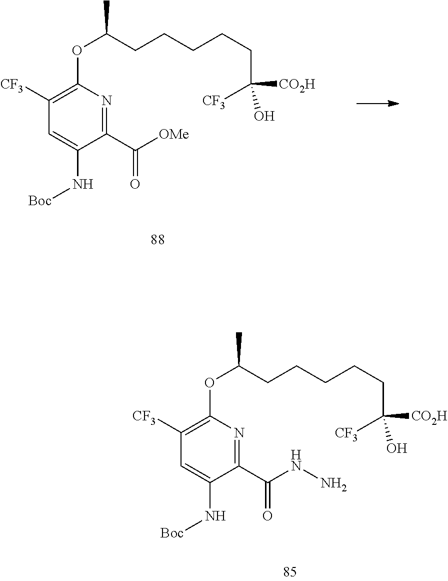 [SMILES: COC(=O)c1nc(O[C@@H](C)CCCCC[C@@](O)(C(=O)O)C(F)(F)F)c(C(F)(F)F)cc1NC(=O)OC(C)(C)C ; C[C@@H](CCCCC[C@@](O)(C(=O)O)C(F)(F)F)Oc1nc(C(=O)NN)c(NC(=O)OC(C)(C)C)cc1C(F)(F)F]

A solution of (2R,8S)-8-({5-[(tert-butoxycarbonyl)amino]-6-(methoxycarbonyl)-3-(trifluoromethyl)pyridin-2-yl}oxy)-2-hydroxy-2-(trifluoromethyl)nonanoic acid (800 mg, 1.39 mmol, 1 equiv.) in t-BuOH (4.8 mL, 6 Vols) was treated with NH₂NH₂H₂O (0.208 g, 202 μL, 4.16 mmol, 3 equiv.) and allowed to stir at RT. After 6 h, TBD (1,5,7-Triazabicyclo[4.4.0]dec-5-ene; 39 mg, 0.28 mmol, 0.2 equiv.) was added and the mixture which was stirred at RT for 4 d. The mixture was partitioned between EtOAc and 0.5 M HCl (aqueous pH ˜2). The phases were separated, and the organic phase was washed with water, 2% NaCl, dried (Na₂SO₄), and concentrated to afford the title compound (848 mg; 106%) as a yellow foam.

### Step 13: tert-Butyl ((3S,9R)-9-hydroxy-3-methyl-10,13-dioxo-1³,9-bis(trifluoromethyl)-2-oxa-11,12-diaza-1(2,6)-pyridinacyclotridecaphane-1⁵-yl)carbamate (86)

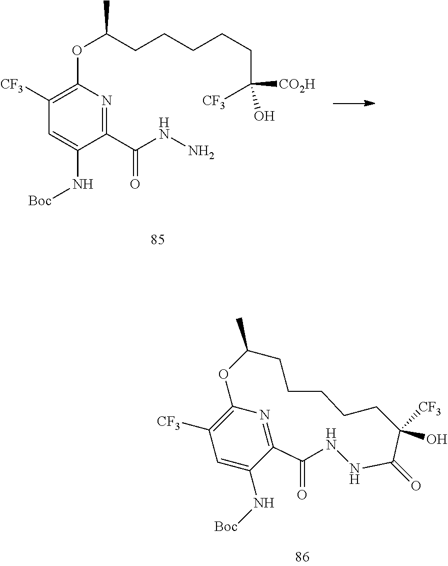 [SMILES: C[C@@H](CCCCC[C@@](O)(C(=O)O)C(F)(F)F)Oc1nc(C(=O)NN)c(NC(=O)OC(C)(C)C)cc1C(F)(F)F ; C[C@H]1CCCCC[C@](O)(C(F)(F)F)C(=O)NNC(=O)c2nc(c(C(F)(F)F)cc2NC(=O)OC(C)(C)C)O1]

A solution of (2R,8S)-8-({5-[(tert-butoxycarbonyl)amino]-6-(hydrazinecarbonyl)-3-(trifluoromethyl)pyridin-2-yl}oxy)-2-hydroxy-2-(trifluoromethyl)nonanoic acid (20 mg, 0.035 mmol, 1 equiv.) in N,N-dimethylformamide (1.6 mL, 80 Vols) was stirred at RT and then a solution of 10% w/v DIPEA in 2-MeTHF (9 mg, 90 μL, 0.069 mmol, 2 equiv.) followed by HATU (1-[Bis(dimethylamino)methylene]-1H-1,2,3-triazolo[4,5-b]pyridinium 3-oxid hexafluorophosphate (20.0 mg, 0.052 mmol, 1.5 equiv.) were added. After 20 h, the mixture was partitioned between water (8 mL)/brine (2 mL) and EtOAc (10 mL). The phases were separated, and the organic phase was washed with 2% brine (2×3 mL) and concentrated to afford 47 mg (wet with water) of a yellow oil. The crude product was purified by silica gel chromatography (4-g SiO₂ cartridge). The column was equilibrated with 5% EtOAc/hexanes and the product was eluted with a gradient from 5 to 50% EtOAc/hexanes. The major peak was concentrated (40° C./20 torr) followed by N₂ stream to afford tert-butyl ((3S,9R)-9-hydroxy-3-methyl-10,13-dioxo-1³,9-bis(trifluoromethyl)-2-oxa-11,12-diaza-1(2,6)-pyridinacyclotridecaphane-1⁵-yl)carbamate (3 mg; 15%) as a yellow-tinted film.

### Step 14: tert-Butyl ((3S,9R)-9-(benzyloxy)-3-methyl-10,13-dioxo-1³,9-bis(trifluoromethyl)-2-oxa-11,12-diaza-1(2,6)-pyridinacyclotridecaphane-1⁵-yl)carbamate (87)

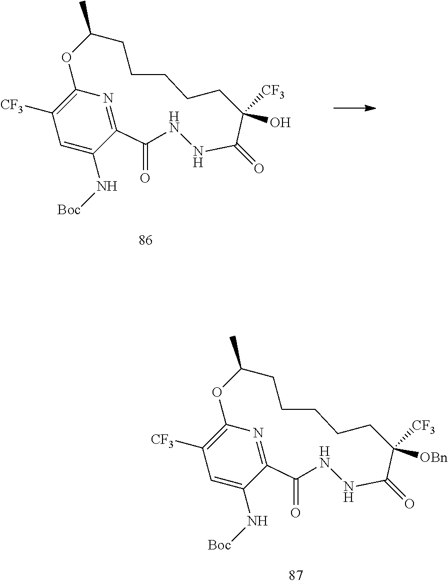 [SMILES: C[C@H]1CCCCC[C@](O)(C(F)(F)F)C(=O)NNC(=O)c2nc(c(C(F)(F)F)cc2NC(=O)OC(C)(C)C)O1 ; C[C@H]1CCCCC[C@](OCc2ccccc2)(C(F)(F)F)C(=O)NNC(=O)c2nc(c(C(F)(F)F)cc2NC(=O)OC(C)(C)C)O1]

tert-Butyl ((3S,9R)-9-(benzyloxy)-3-methyl-10,13-dioxo-1³,9-bis(trifluoromethyl)-2-oxa-11,12-diaza-1(2,6)-pyridinacyclotridecaphane-15-yl)carbamate is synthesized in a manner analogous to Example 23, Intermediate 2, Step 3.

### Step 15: tert-Butyl ((4S,10R)-10-(benzyloxy)-4-methyl-2⁵,10-bis(trifluoromethyl)-3-oxa-1(2,5)oxadiazola-2(2,6)-pyridinacyclodecaphane-2³-yl)carbamate (70)

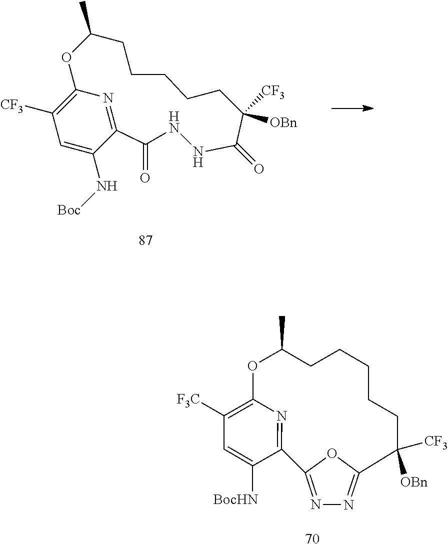 [SMILES: C[C@H]1CCCCC[C@](OCc2ccccc2)(C(F)(F)F)C(=O)NNC(=O)c2nc(c(C(F)(F)F)cc2NC(=O)OC(C)(C)C)O1 ; C[C@H]1CCCCC[C@](OCc2ccccc2)(C(F)(F)F)C2=NN=C(O2)c2nc(c(C(F)(F)F)cc2NC(=O)OC(C)(C)C)O1]

tert-Butyl ((4S,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate is synthesized in a manner analogous to Example 24, Step 6.

tert-Butyl ((4R,10R)-10-(benzyloxy)-4-methyl-25,10-bis(trifluoromethyl)-3-oxa-1(2,5)-oxadiazola-2(2,6)-pyridinacyclodecaphane-23-yl)carbamate (64):

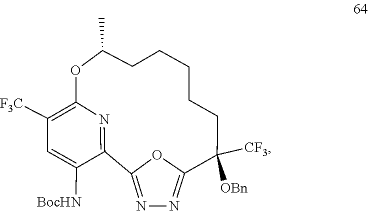 [SMILES: C[C@@H]1CCCCC[C@](OCc2ccccc2)(C(F)(F)F)C2=NN=C(O2)c2nc(c(C(F)(F)F)cc2NC(=O)OC(C)(C)C)O1]

is prepared in a manner analogous to the product of Example 29, Step 15.

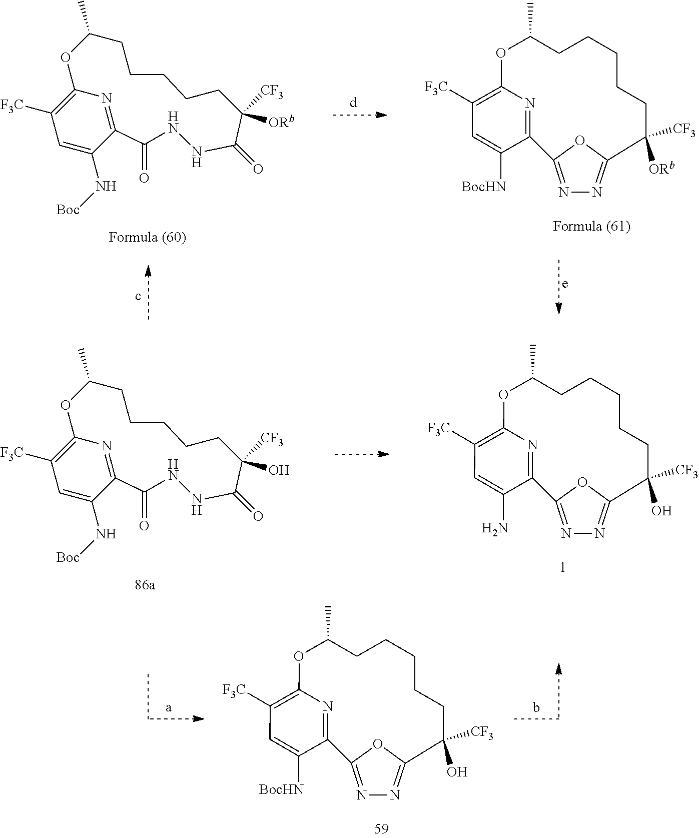

Compound I can be prepared by converting compound 86a into compound I. Compound I can be prepared by converting compound 59 into compound I. Compound I can be prepared by converting Formula (61) into compound I. Compound I can be prepared by converting Formula (60) into compound I.

In some embodiments, compound 59 can be prepared by converting compound 86a into compound 59. In some embodiments, compound Formula (61) can be prepared by converting Formula (60) into Formula (61). In some embodiments, Formula (60) can be prepared by converting compound 86a into Formula (60).

In some embodiments, compound I can be prepared by converting compound 86a into compound 59, followed by converting compound 59 into compound I.

In some embodiments, compound I can be prepared by converting compound 86a into Formula (60), followed by converting Formula (60) into Formula (61), followed by converting compound Formula (61) into compound I.

In some embodiments, step a) can be performed in the presence of acetic anhydride or trifluoroacetic anhydride.

In some embodiments, step b) can be performed in the presence of an acid. In some embodiments, the acid can be selected from trifluoroacetic acid (TFA), hydrochloric acid (HCl), methanesulfonic acid (MsOH), phosphoric acid (H₃PO4), and sulfuric acid (H₂SO₄). In some embodiments, the acid can be trifluoroacetic acid (TFA).

In some embodiments, step c) can be performed in the presence of a base and an alkyl halide, alkyl triflate, or alkyl tosylate. In some embodiments, the base can be selected from lithium carbonate (Li₂CO₃), sodium carbonate (Na₂CO₃), potassium carbonate (K₂CO₃), cesium carbonate (Cs₂CO₃), sodium tert-butoxide (NaOt-Bu), and potassium tert-butoxide (KOt-Bu). In some embodiments, the alkyl halide can be a benzyl halide and R^{b} can be Bn. In some embodiments, the benzyl halide can be selected from benzyl chloride (BnCl), benzyl bromide (BnBr), and benzyl iodide (BnI). In some embodiments, the base can be cesium carbonate (Cs₂CO₃), the alkyl halide can be benzyl bromide, and R^{b} can be Bn.

In some embodiments, step d) can be performed in the presence of a sulfonyl chloride and a base. In some embodiments, the sulfonyl chloride can bep-toluenesulfonyl chloride (TsCl). In some embodiments, the base can be an amine base. In some embodiments, the amine base can be N,N,diisopropylethylamine (DIPEA). In some embodiments, the amine base can be triethylamine (TEA). In some embodiments, R^{b} can be benzyl, the sulfonyl chloride can bep-toluenesulfonyl chloride (TsCl), and the amine base can be N,N,diisopropylethylamine (DIPEA).

In some embodiments, step e) can performed be in one step.

In some embodiments, step e) can performed in two steps. In some embodiments, R^{b} can be Bn and step e-1) can be performed in the presence of reducing conditions. In some embodiments, the reducing conditions can be hydrogen gas (H₂) and palladium on carbon (Pd/C). In some embodiments, R^{b} can be Bn and step e-2) can be performed in the presence of an acid. In some embodiments, the acid can be selected from trifluoroacetic acid (TFA), hydrochloric acid (HCl), methanesulfonic acid (MsOH), phosphoric acid (H₃PO₄), and sulfuric acid (H₂SO₄). In some embodiments, the acid can be trifluoracetic acid (TFA).

### Example 30: Synthesis of tert-Butyl ((4R,10R)-10-hydroxy-4-methyl-25,10-bis(trifluoromethyl)-3-oxa-1(2,5)-oxadiazola-2(2,6)-pyridinacyclodecaphane-23-yl)carbamate (59)

### Step 1: Methyl 6-(allyloxy)-3-nitro-5-(trifluoromethyl)picolinate (89)

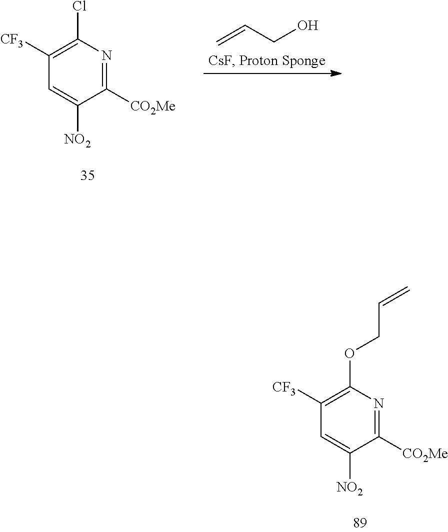

Methyl 6-chloro-3-nitro-5-(trifluoromethyl)picolinate (17.14 g, 60.23 mmol) was dissolved in acetonitrile (429 mL). 2-propen-1-ol (4.92 mL, 72.3 mmol), and N,N,N′,N′-tetramethyl-1,8-naphthalenediamine (15.5 g, 72.3 mmol) and cesium fluoride (10.5 g, 69.3 mmol) were added to the reaction flask. The reaction was stirred at room temperature overnight at which time the reaction was complete. The mixture was cooled with an ice bath and filtered to remove the solids. The solids were rinsed with acetonitrile. The reaction was concentrated in which least ½ of the acetonitrile was removed. The reaction mixture was diluted with 2-MeTHF (220 mL) and washed with 1.0 M HCl (171 mL) in two portions and with water (171 mL) in two portions. The mixture was concentrated to an oil to isolate methyl 6-(allyloxy)-3-nitro-5-(trifluoromethyl)picolinate as a low melting solid (15.4 g) in an 83.5% yield.

¹H NMR (400 MHz, CDCl₃) δ ppm 4.04 (s, 3H) 5.09 (dt, J=5.47, 1.36 Hz, 2H) 5.35 (dd, J=10.51, 1.22 Hz, 1H) 5.47 (dq, J=17.18, 1.41 Hz, 1H) 5.91-6.18 (m, 1H) 8.67 (s, 1H); ¹⁹F NMR (400 MHz, CDCl₃) δ ppm −64.45 (s, 3F).

### Step 2: Methyl 6-(allyloxy)-3-amino-5-(trifluoromethyl)picolinate (90)

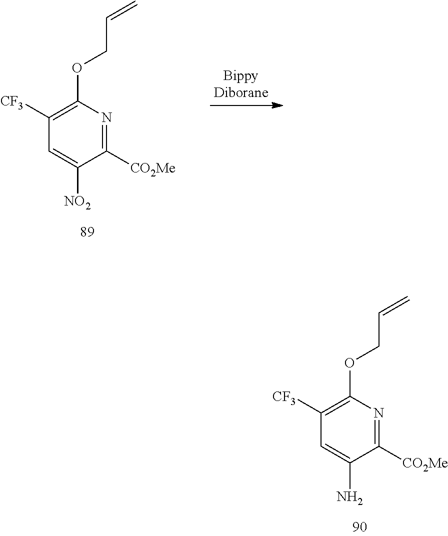 [SMILES: C=CCOc1nc(C(=O)OC)c([N+](=O)[O-])cc1C(F)(F)F ; C=CCOc1nc(C(=O)OC)c(N)cc1C(F)(F)F]

To a 3-neck RBF was added tetrahydroxydiboron (9.662 g, 107.8 mmol) and N,N-dimethylformamide (40 mL) and this was stirred at room temperature. To another flask was added 4,4′-bipyridine (28.05 mg, 0.1796 mmol), methyl 6-(allyloxy)-3-nitro-5-(trifluoromethyl)picolinate (10.00 g, 32.66 mmol) and N,N-dimethylformamide (10 mL). The substrate/catalyst solution was slowly added to the tetrahydroxydiboron/DMF solution. The addition was done at 15-20° C. but there was a delayed exotherm to 75° C. After cooling back to 30° C., the remainder of the addition at 30-40° C. with the jacket at 10° C. did not show a delayed exotherm and the addition was complete in 20 minutes. No exotherm was noted after the addition was complete. To maintain the internal temperature between 30-40° C., the jacket was set to 30° C. When the reaction was complete, product was precipitated by adding 10% aq. NaCl (100 mL) at 30° C. The mixture was stirred for a few minutes, crystallized instantly, filtered, washed with water to isolate methyl 6-(allyloxy)-3-amino-5-(trifluoromethyl)picolinate (7.8 g) in an 87% chemical yield.

¹H NMR (400 MHz, DMSO-d6) δ ppm 3.83 (s, 3H) 4.70-4.94 (m, 2H) 5.22 (dd, J=10.58, 1.53 Hz, 1H) 5.38 (dd, J=17.24, 1.71 Hz, 1H) 6.04 (ddt, J=17.18, 10.51, 5.10, 5.10 Hz, 1H) 6.61 (br. s., 2H) 7.74 (s, 1H); ¹⁹F NMR (400 MHz, DMSO-d6) δ ppm 63.01 (s, 3 F); MS (ESI+) for C₁₁H₁₁F₃N₂O₃ m/z 277.0 (M+H)⁺ and m/z 299.0 (M+Na)⁺.

### Step 3: 6-(Allyloxy)-3-amino-5-(trifluoromethyl)picolinic acid (91)

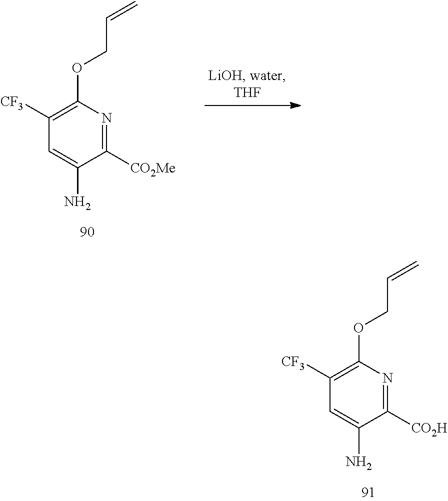 [SMILES: C=CCOc1nc(C(=O)OC)c(N)cc1C(F)(F)F ; C=CCOc1nc(C(=O)O)c(N)cc1C(F)(F)F]

6-(Allyloxy)-3-amino-5-(trifluoromethyl)picolinic acid can be prepared using a procedure similar to Example 23, Intermediate 1, Step 7.

### Step 4: 6-(Allyloxy)-3-((tert-butoxycarbonyl)amino)-5-(trifluoromethyl)picolinic acid (92)

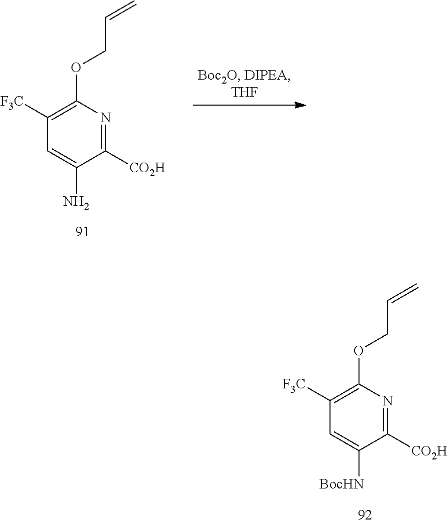 [SMILES: C=CCOc1nc(C(=O)O)c(N)cc1C(F)(F)F ; C=CCOc1nc(C(=O)O)c(NC(=O)OC(C)(C)C)cc1C(F)(F)F]

To (R)-3-amino-6-(pent-4-en-2-yloxy)-5-(trifluoromethyl)picolinic acid oil was added tetrahydrofuran (360 mL, 7.0 vol.), N,N-diisopropylethylamine (32.22 mL, 185.0 mmol, 1.0 eq.), Boc₂O (12.11 g, 55.50 mmol, 0.3 eq.) and heated to reflux overnight. The mixture was cooled in an ice bath, added HCl (453.5 mL, 222.0 mmol, 1.20 eq., 0.5 M) and heptane (360 mL, 7.0), removed the aqueous phase, washed with water (150 mL, 3 vol.), removed the aqueous phase, and concentrated on a rotovap at RT to oil. Added acetonitrile (250 mL, 5 vol.), concentrated on a rotovap to azeotrope off water to oil. Carried forward to the next synthetic steps “as is” 86% purity.

¹H NMR (400 MHz, CDCl₃) δ ppm 1.56 (s, 9H) 4.94 (dt, J=5.14, 1.59 Hz, 2H) 5.32-5.39 (m, 1H) 5.42-5.57 (m, 1H) 5.95-6.17 (m, 1H) 9.33 (s, 1H) 10.00 (br. s., 1H) 10.63 (br. s., 1H)

### Step 5: tert-Butyl (6-(allyloxy)-2-(2-((2R,8S)-2-(benzyloxy)-8-hydroxy-2-(trifluoromethyl)nonanoyl)hydrazine-1-carbonyl)-5-(trifluoromethyl)pyridin-3-yl)carbamate (93)

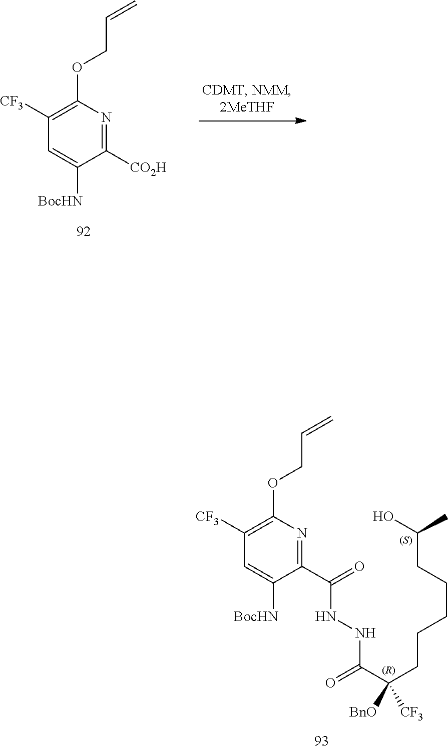

tert-Butyl (6-(allyloxy)-2-(2-((2R,8S)-2-(benzyloxy)-8-hydroxy-2-(trifluoromethyl)nonanoyl)hydrazine-1-carbonyl)-5-(trifluoromethyl)pyridin-3-yl)carbamate can be prepared using a procedure similar to Example 24, Step 5.

### Step 6: tert-Butyl (6-(allyloxy)-2-(5-((2R,8S)-2-(benzyloxy)-1,1,1-trifluoro-8-hydroxynonan-2-yl)-1,3,4-oxadiazol-2-yl)-5-(trifluoromethyl)pyridin-3-yl)carbamate (94)

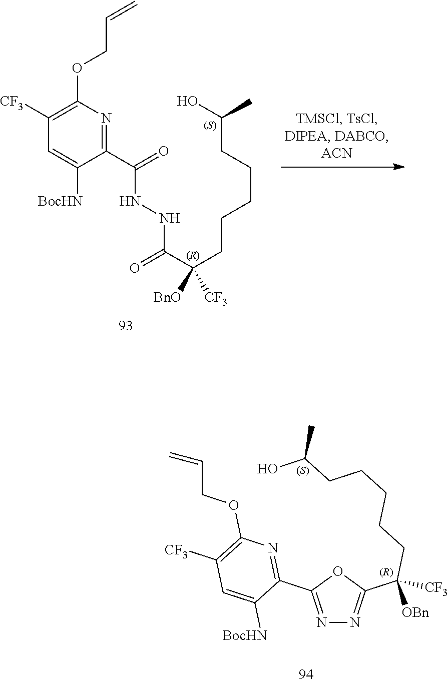

tert-Butyl (6-(allyloxy)-2-(5-((2R,8S)-2-(benzyloxy)-1,1,1-trifluoro-8-hydroxynonan-2-yl)-1,3,4-oxadiazol-2-yl)-5-(trifluoromethyl)pyridin-3-yl)carbamate can be prepared using a procedure similar to Example 24, Step 6.

### Step 7: (2S,8R)-8-(Benzyloxy)-8-(5-(3-((tert-butoxycarbonyl)amino)-6-hydroxy-5-(trifluoromethyl)pyridin-2-yl)-1,3,4-oxadiazol-2-yl)-9,9,9-trifluorononan-2-yl 4-methylbenzenesulfonate (96)

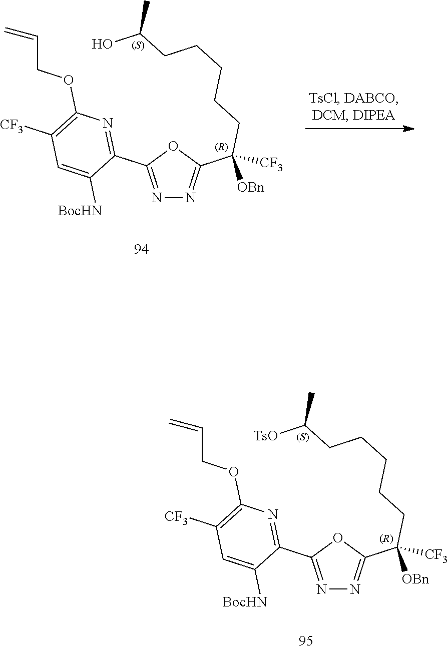

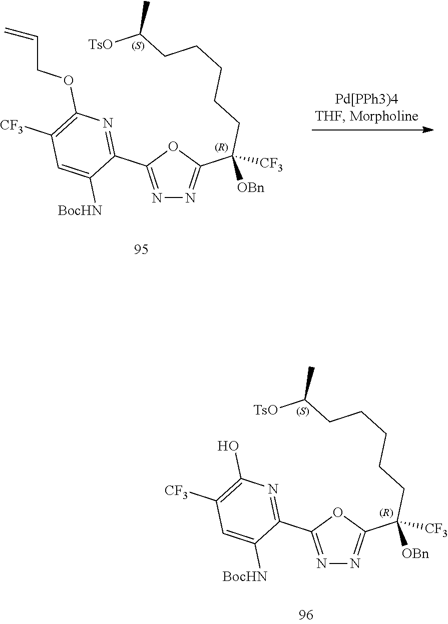

To a 3-neck RBF was added tert-butyl (6-(allyloxy)-2-(5-((2R,8S)-2-(benzyloxy)-1,1,1-trifluoro-8-hydroxynonan-2-yl)-1,3,4-oxadiazol-2-yl)-5-(trifluoromethyl)pyridin-3-yl)carbamate (71.40 g, 101.0 mmol) DCM (360 mL, 5 vol.) and the reaction was placed in a water bath. Added N,N-diisopropylethylamine (83.6 mL, 479.9 mmol, 4.75 eq.). To another flask was added p-Toluenesulfonyl chloride (52.97 g, 277.9 mmol, 2.75 eq.) and DCM (360 mL, 5 vol.). This was stirred to give a solution. The TsCl solution was added all at once to the reaction. The reaction was stirred at RT overnight. The reaction was cooled in an ice bath and DABCO (5.67 g, 50.52 mmol, 0.5 eq.) was added at 6° C. internal temperature. The reaction was exothermic, and the internal temperature reached 15° C. Added 2.0 M of potassium hydroxide in water (50.5 mL, 101.0 mmol, 1 eq.) and stirred at 10° C. for 10 minutes. Added morpholine (17.6 mL, 202.0 mmol, 2 eq.) and bubbled nitrogen into the solvent for a few minutes. Added tetrakis(triphenylphosphine)palladium(0) (1.17 g, 1.010 mmol), 0.01 eq.) and stirred for 20 minutes. Added water (150 mL, 2 vol.), removed aqueous phase, added water (200 mL, 3 vol.), removed the aqueous phase. Added 1.0 M aq. HCl (350 mL, 3.5 eq., 5 vol.), removed the aqueous phase, washed the organic phase with water (250 ml, 3 vol.), and removed the aqueous phase. Concentrated the organic phase on a rotovap to oil. Added toluene (350 ml, 5 vol.), filtered via a 3-inch plug of Magnesol. Concentrated the toluene solution to about 4 volumes to remove residual DCM. Carried forward as a toluene solution, 320 g, qNMR=25.1 wt % product or 80.3 g, 99% yield.

¹H NMR (400 MHz, CDCl₃) δ ppm 1.22 (d, J=6.24 Hz, 3H) 1.25-1.40 (m, 3H) 1.48-1.71 (m, 15H) 2.32-2.42 (m, 2H) 2.45 (s, 3H) 4.64 (m, 1H) 4.69 (d, J=10.88 Hz, 1H) 4.85 (d, J=10.88 Hz, 1H) 7.24-7.45 (m, 7H) 7.80 (d, J=8.31 Hz, 2H) 9.24 (s, 1H) 9.78 (s, 1H).

### Step 8: tert-Butyl ((4R,10R)-10-(benzyloxy)-4-methyl-2⁵,10-bis(trifluoromethyl)-3-oxa-1(2,5)-oxadiazola-2(2,6)-pyridinacyclodecaphane-2³-yl)carbamate (64)

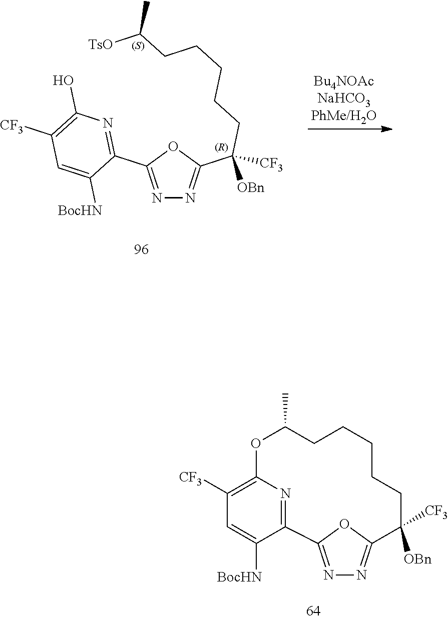

To a jacketed RBF was added toluene (210 mL, 3 vol.) sodium bicarbonate (14.12 g, 168.04 mmol, 2.0 eq.), tetrabutylammonium acetate (27.87 g, 92.42 mmol, 1.10 eq.) contains acetic acid (2.78 g, 46.3 mmol, 0.55 eq) 10% by weight and water (470 mL, 7 vol.). This solution was heated to 78° C. To another RBF was added (2S,8R)-8-(benzyloxy)-8-(5-(3-((tert-butoxycarbonyl)amino)-6-hydroxy-5-(trifluoromethyl)pyridin-2-yl)-1,3,4-oxadiazol-2-yl)-9,9,9-trifluorononan-2-yl 4-methylbenzenesulfonate (71.00 g, 84.02 mmol, 1.0 eq.) and toluene (1080 mL, 15 vol.) at 47.5 ml/hr for a target of 24 h. The solution was added with pump and stirred with a mechanical stirrer. The reaction was cooled to 30-40° C. and the aqueous phase was removed. The organic phase was washed with 0.5 M HCl (140 ml, 2 vol.) at 30-40° C. The aqueous phase was removed, and the organic wash was washed with water (3 vol.) at 30-40° C. The aqueous phase was removed. The organic phase was washed with 5 wt % NaHCO₃ (140 mL, 2 vol.) at 30-40° C., the aqueous phase was removed. The organic phase was washed with water (280 mL, 4 vol.) at 30-40° C. and the aqueous phase was removed. The organic phase was concentrated on a rotovap to 10 volumes or 710 mL and filtered via a 3-inch plug of silica gel. The silica gel was washed with toluene (75 mL 1 vol.). Concentrated the filtrate to 2 volumes on a rotovap, added 4 volumes of EtOH, concentrated to 2 volumes under partial vacuum, added 4 volumes of EtOH, concentrated to 2 vol, added 1 vol of EtOH. Heated to reflux, slowly cooled to RT, cooled to 10° C., aged for 1 h, filtered the slurry, washed with 10° C. EtOH (75 mL, 1 vol.×2). Dried the solids in a vacuum oven at 50° C. to isolate 29 g or 55% yield of tert-butyl ((4R,10R)-10-(benzyloxy)-4-methyl-25,10-bis(trifluoromethyl)-3-oxa-1(2,5)-oxadiazola-2(2,6)-pyridinacyclodecaphane-23-yl)carbamate.

¹H NMR (400 MHz, CDCl₃) δ ppm 0.76-0.93 (m, 1H) 1.25-1.37 (m, 2H) 1.47 (d, J=6.36 Hz, 3H) 1.57 (s, 9H) 1.62-1.87 (m, 4H) 2.24-2.35 (m, 1H) 2.49-2.61 (m, 2H) 4.66-4.78 (m, 2H) 4.86-4.97 (m, 1H) 7.29-7.39 (m, 5H) 9.12 (s, 1H) 9.23 (s, 1H).

### Step 9: tert-Butyl ((4R,10R)-10-hydroxy-4-methyl-2⁵,10-bis(trifluoromethyl)-3-oxa-1(2,5)-oxadiazola-2(2,6)-pyridinacyclodecaphane-2³-yl)carbamate (59)

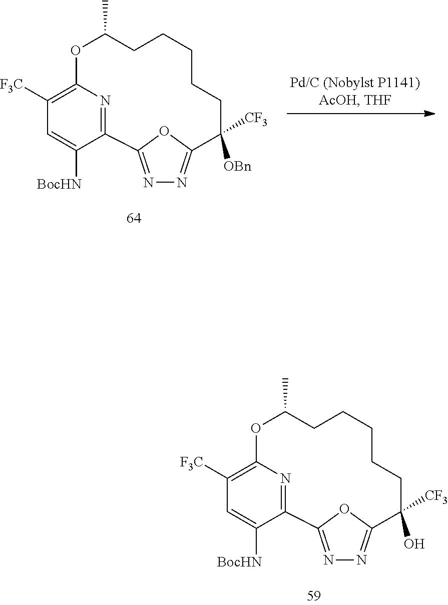

tert-Butyl ((4R,10R)-10-hydroxy-4-methyl-2⁵,10-bis(trifluoromethyl)-3-oxa-1(2,5)-oxadiazola-2(2,6)-pyridinacyclodecaphane-2³-yl)carbamate can be prepared using a procedure similar to Example 25, Step 8.

### Example 31: Synthesis of (2R,8S)-2-(Benzyloxy)-8-hydroxy-2-(trifluoromethyl)nonanehydrazide (97)

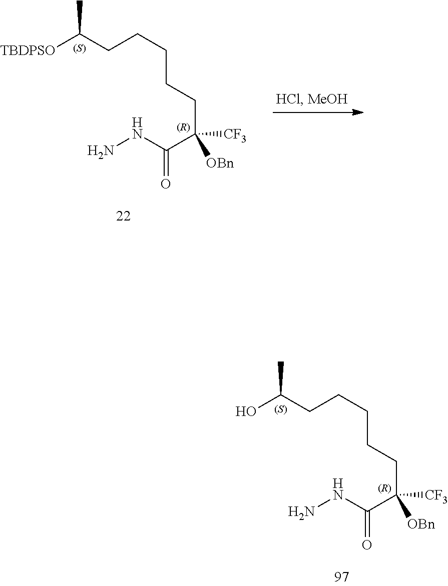

To a 3-neck RBF was added (2R,8S)-2-(benzyloxy)-8-((tert-butyldiphenylsilyl)oxy)-2-(trifluoromethyl)nonanehydrazide. To another flask was added IPA (684 mL) and 12 M HCl aqueous (340 mL, 4100 mmol) and were mixed. The IPA/HCl aqueous solution was added to the (2R,8S)-2-(benzyloxy)-8-((tert-butyldiphenylsilyl)oxy)-2-(trifluoromethyl)nonanehydrazide in a 3-neck RBF. The oil did not dissolve so the mixture was heated to 60° C. After reaching 60° C. most of the oil had dissolved. The reaction was held at 60° C. for 1 hour. An HPLC sample was taken and diluted in ACN. The reaction was cooled to RT, concentrated on a rotovap under vacuum to remove the IPA and water by azeotrope. To the mixture was added water (1000 mL). The mixture was extracted by adding hexane (330 mL) and separating in a sep. funnel. This was repeated two additional times with hexane (330 mL) to remove the TBDPSOH and hexane soluble impurities. The hexane extracts were discarded. The aqueous phase was cooled in an ice water bath and 47% KOH in water (11.7 M, 211, 2470 mmol) was added with an addition funnel while keeping the temperature <10° C. The pH was checked to be >10 with pH paper. The water was extracted with 2-MeTHF (510 mL) and the phases were separated in a sep funnel. The aqueous phase was discarded. The 2-MeTHF extract was washed with water (330 mL) and the aqueous phase was removed in a sep. funnel. The water was discarded and the 2-MeTHF extract was concentrated on a rotovap to oil. To the oil was added toluene (330 mL) and concentrated on a rotovap to oil again to azeotrope off residual water. (2R,8S)-2-(benzyloxy)-8-hydroxy-2-(trifluoromethyl)nonanehydrazide.

¹H NMR (400 MHz, CDCl₃) δ ppm 1.21 (d, J=6.11 Hz, 3H) 1.23-1.56 (m, 10H) 2.05-2.20 (m, 1H) 2.27-2.37 (m, 1H) 3.76-3.86 (m, 1H) 4.62 (d, J=10.52 Hz, 1H) 4.77 (d, J=10.39 Hz, 1H) 7.11-7.24 (m, 2H) 7.31-7.48 (m, 3H) 7.79-8.21 (m, 1H). MS: calculated M+H⁺=363.19, Found M+H⁺=363.2, M+H⁺−H₂O=345.2.

### Example 32: Alternative Synthesis of tert-Butyl (2-(5-((R)-2-(benzyloxy)-1,1,1-trifluorohex-5-en-2-yl)-1,3,4-oxadiazol-2-yl)-6-(((R)-pent-4-en-2-yl)oxy)-5-(trifluoromethyl)pyridin-3-yl)carbamate (48)

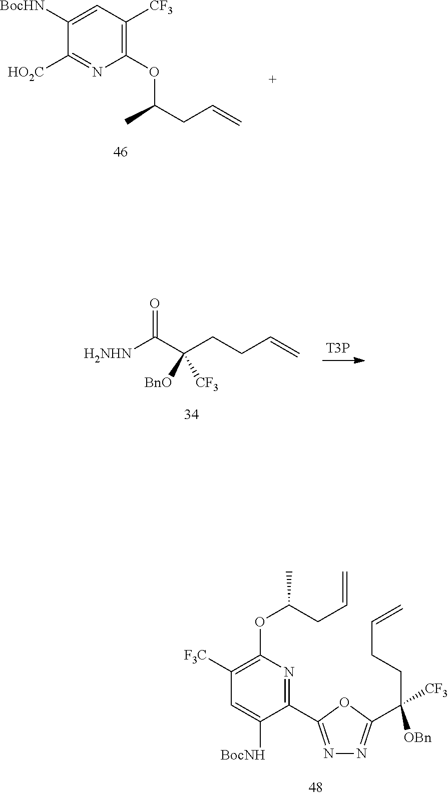

tert-Butyl (2-(5-((R)-2-(benzyloxy)-1,1,1-trifluorohex-5-en-2-yl)-1,3,4-oxadiazol-2-yl)-6-(((R)-pent-4-en-2-yl)oxy)-5-(trifluoromethyl)pyridin-3-yl)carbamate can be synthesized using a procedure similar to those reported in:
Li, C.; Dickson, H. D. Tett. Lett. 2009, 50, 6435.
Augustine, J. K.; Vairaperumal, V.; Narasimhan, S.; Alagarsamy, P.; Radhakrishnan, A. Tetrahedron, 2009, 65, 9989.

A suitable vessel with overhead stirring and nitrogen inlet is charged with (R)-3-((tert-butoxycarbonyl)amino)-6-(pent-4-en-2-yloxy)-5-(trifluoromethyl)picolinic acid (5.8 g, 14.9 mmol, basis), (R)-2-(benzyloxy)-2-(trifluoromethyl)hex-5-enehydrazide (5.0 g, 15.6 mmol, 1.05 eq), and acetonitrile (34.8 mL, 6 vol). The mixture is agitated, and T₃P (33.1 g, 50 w/w %, 52.0 mmol, 3.5 eq) and DIPEA (11.5 gm 89.2 mmol, 6 eq) is charged to the reactor. The mixture is heated to an internal temperature of 78° C. and monitored by HPLC until desired reaction conversion is met. The temperature is lowered to 25° C. and water (29 mL, 5 vol) is charged to the reactor and the mixture is stirred for 10 minutes. MTBE (46.4 mL, 8 vol) is charged, stirred for 10 minutes, settled and separated. The organic layer is subsequently washed with 5% citric acid (29 mL, 5 vol), saturated sodium bicarbonate (29 mL, 5 vol), and water (29 mL, 5 vol). The organic layer is concentrated to an oil and used as is in subsequent processing.

### Example 33: Alternative Synthesis of tert-Butyl (2-(5-((R)-2-(benzyloxy)-1,1,1-trifluorohex-5-en-2-yl)-1,3,4-oxadiazol-2-yl)-6-(((R)-pent-4-en-2-yl)oxy)-5-(trifluoromethyl)pyridin-3-yl)(tert-butoxycarbonyl)carbamate (49)

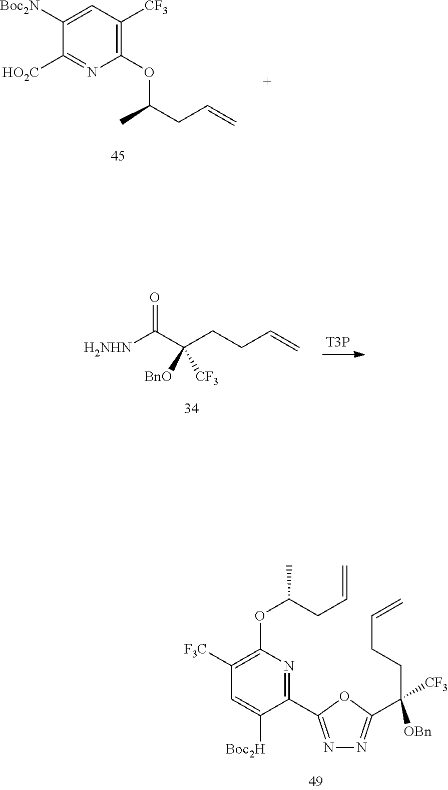

tert-Butyl (2-(5-(2-(benzyloxy)-1,1,1-trifluorohex-5-en-2-yl)-1,3,4-oxadiazol-2-yl)-6-(((R)-pent-4-en-2-yl)oxy)-5-(trifluoromethyl)pyridin-3-yl)(tert-butoxycarbonyl)carbamate can be synthesized using a procedure analogous to that of Example 32.

### Example 34: Synthesis of (R)-3-Nitro-6-(pent-4-en-2-yloxy)-5-(trifluoromethyl)picolinic acid (98)

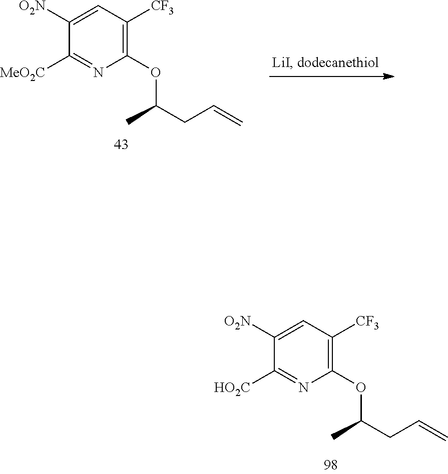 [SMILES: C=CC[C@@H](C)Oc1nc(C(=O)OC)c([N+](=O)[O-])cc1C(F)(F)F ; C=CC[C@@H](C)Oc1nc(C(=O)O)c([N+](=O)[O-])cc1C(F)(F)F]

Methyl (R)-3-nitro-6-(pent-4-en-2-yloxy)-5-(trifluoromethyl)picolinate (1.23 g, 3.68 mmol) was charged to a round bottom flask and dissolved in ethyl acetate (8.6 mL). The solution was degassed subsurface with nitrogen sparging while charging lithium iodide (0.542 g, 4.05 mmol) and 1-dodecanethiol (1.19 mL, 4.97 mmol). The mixture was held under nitrogen and heated to near reflux (oil bath at 80° C.) for 9 hours. The solution was allowed to cool to 50° C. and heptane (25.8 mL) was added. The mixture had just clouded at the end of the addition but the mixture became a clear solution when addition stopped. The mixture was allowed to cool as oil bath cooled, noting solids at about 35° C. The resulting thick slurry was stirred for 1 hour at ambient temperature and then filtered. The product was washed with heptane (3×8.6 mL). After drying the solids lithium (R)-3-nitro-6-(pent-4-en-2-yloxy)-5-(trifluoromethyl)picolinate (1.13 g) was isolated in a 94.1%. ¹H NMR (400 MHz, MeOH-d4) δ (ppm)=8.65 (s, 1H), 5.87 (tdd, J=7.0, 10.2, 17.2 Hz, 1H), 5.69-5.45 (m, 1H), 5.29-4.95 (m, 2H), 2.68-2.27 (m, 2H), 1.45-1.36 (m, 3H). ¹⁹F NMR (376 MHz, MeOH-d4) δ (ppm)=−65.44. MS (ESI-) for C₁₂H₁₀F₃LiN₂O₅ m/z 275.1 (MLiCO₂—H)⁻.

### Example 35: Alternative Synthesis of (4R,10R)-2³-Amino-4-methyl-2⁵,10-bis(trifluoromethyl)-3-oxa-1(2,5)-oxadiazola-2(2,6)-pyridinacyclodecaphan-10-ol (Compound I)

### Step 1: 2-((R)-2-(Benzyloxy)-1,1,1-trifluorohex-5-en-2-yl)-5-(3-nitro-6-(((R)-pent-4-en-2-yl)oxy)-5-(trifluoromethyl)pyridin-2-yl)-1,3,4-oxadiazole (99)

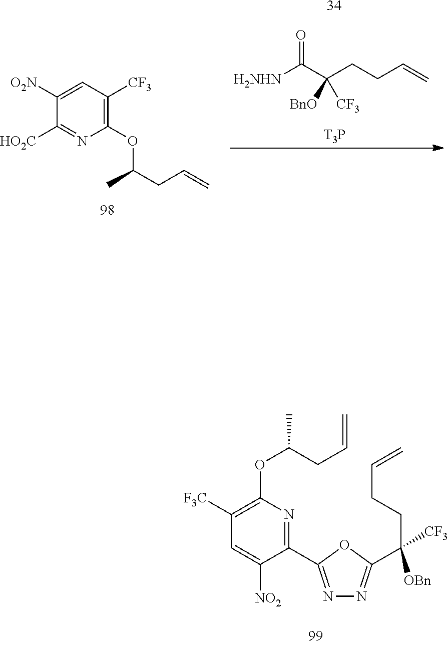

2-((R)-2-(Benzyloxy)-1,1,1-trifluorohex-5-en-2-yl)-5-(3-nitro-6-(((R)-pent-4-en-2-yl)oxy)-5-(trifluoromethyl)pyridin-2-yl)-1,3,4-oxadiazole can be synthesized using a procedure similar to that of Example 32.

¹H NMR (400 MHz, MeOH-d4) δ (ppm)=8.81 (s, 1H), 7.49-7.19 (m, 5H), 5.93-5.73 (m, 2H), 5.61-5.45 (m, 1H), 5.18-4.97 (m, 4H), 4.83-4.65 (m, 2H), 2.62-2.42 (m, 4H), 2.42-2.17 (m, 2H), 1.41 (d, J=6.2 Hz, 3H). ¹⁹F NMR (376 MHz, MeOH-d4) δ (ppm)=−74.90 (s, 3F), −65.91 (s, 3F). MS (ESI+) for C₂₆H₂₄F₆N₄O₅ m/z 587.2 (M+H)⁺.

### REFERENCES

Li, C.; Dickson, H. D. Tett. Lett. 2009, 50, 6435.
Augustine, J. K.; Vairaperumal, V.; Narasimhan, S.; Alagarsamy, P.; Radhakrishnan, A. Tetrahedron, 2009, 65, 9989.

### Step 2: (4R,10R,Z)-10-(Benzyloxy)-4-methyl-2³-nitro-2⁵,10-bis(trifluoromethyl)-3-oxa-1(2,5)-oxadiazola-2(2,6)-pyridinacyclodecaphan-6-ene (100)

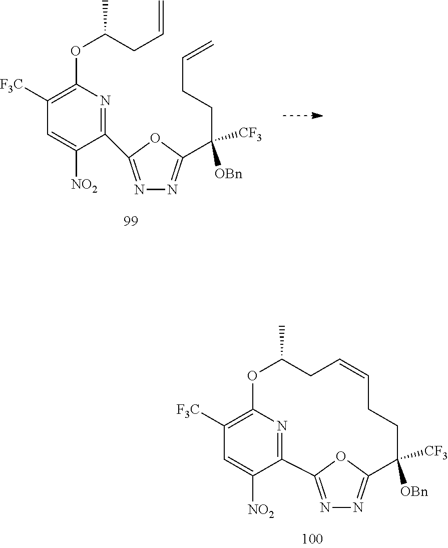 [SMILES: C=CCC[C@@](OCc1ccccc1)(c1nnc(-c2nc(O[C@H](C)CC=C)c(C(F)(F)F)cc2[N+](=O)[O-])o1)C(F)(F)F ; C[C@@H]1C/C=C\CC[C@](OCc2ccccc2)(C(F)(F)F)C2=NN=C(O2)c2nc(c(C(F)(F)F)cc2[N+](=O)[O-])O1]

(4R,10R,Z)-10-(Benzyloxy)-4-methyl-2³-nitro-2⁵,10-bis(trifluoromethyl)-3-oxa-1(2,5)-oxadiazola-2(2,6)-pyridinacyclodecaphan-6-ene can be synthesized using a procedure analogous to Example 24, Step 8.

### Step 3: (4R,10R)-2³-Amino-4-methyl-2⁵,10-bis(trifluoromethyl)-3-oxa-1(2,5)-oxadiazola-2(2,6)-pyridinacyclodecaphan-10-ol (Compound I)

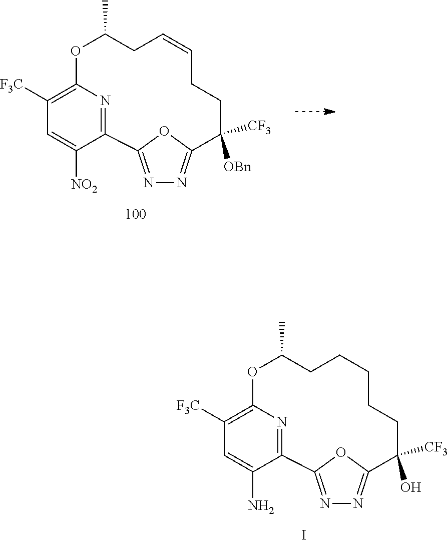

(4R,10R)-2³-Amino-4-methyl-2⁵,10-bis(trifluoromethyl)-3-oxa-1(2,5)-oxadiazola-2(2,6)-pyridinacyclodecaphan-10-ol (Compound I) can be synthesized using a procedure analogous to the following.

A mixture of (4R,10R,Z)-10-(benzyloxy)-4-methyl-2³-nitro-2⁵,10-bis(trifluoromethyl)-3-oxa-1(2,5)-oxadiazola-2(2,6)-pyridinacyclodecaphan-6-ene and 10 wt % palladium on activated carbon (Evonik Nobylst P1173; 50 w/w %, 0.5 equiv.) in EtOH (15 VolEq), EtOAc (5 VolEq), and 7 M NH₃/MeOH (1 equiv.) is evacuated and backfilled with N₂ three times. The reaction vessel is evacuated and backfilled with H₂ (balloon pressure) and rapidly stirred at RT for 16 h. The reaction vessel is evacuated/backfilled with N₂ three times and the mixture is analyzed by LC for reaction completion. A portion of diatomaceous earth is added to the reaction mixture and the suspension is filtered through a 1-cm diatomaceous earth-packed bed to remove the catalyst. The flask/filter-bed is washed with EtOH and the washings are combined with the filtrate and concentrated (45° C./20 torr) to afford crude (4R,10R)-2³-amino-4-methyl-2⁵,10-bis(trifluoromethyl)-3-oxa-1(2,5)-oxadiazola-2(2,6)-pyridinacyclodecaphan-10-ol.

The crude product is dissolved in DCM (7 VolEq) and filtered through a SiO₂-packed bed (1 g/g). The filter-bed is washed with DCM and then 20% EtOAc/DCM. The filtrate and washings are combined and concentrated. The resulting product is dissolved in warm (30° C.) DCM (7 VolEq) and diluted with heptane (7 VolEq) with rapid mixing. The solution is concentrated (45° C./360 torr), to remove most of the DCM, and then the resultant suspension is backfilled with an additional portion of heptane (7 Vols). The suspension is partially concentrated again (to remove residual DCM) and cooled to RT. The resulting product is collected by filtration and the filter-cake is washed with heptane and air-dried to yield (4R,10R)-2³-amino-4-methyl-2⁵,10-bis(trifluoromethyl)-3-oxa-1(2,5)-oxadiazola-2(2,6)-pyridinacyclodecaphan-10-ol.

(4R,10R)-2³-Amino-4-methyl-2⁵,10-bis(trifluoromethyl)-3-oxa-1(2,5)-oxadiazola-2(2,6)-pyridinacyclodecaphan-10-ol (Compound I) can also be synthesized using a procedure analogous to Example 25, Step 8.

### Example 36: Alternative Synthesis of (4R,10R,Z)-10-(Benzyloxy)-4-methyl-2³-nitro-2⁵,10-bis(trifluoromethyl)-3-oxa-1(2,5)-oxadiazola-2(2,6)-pyridinacyclodecaphan-6-ene (100)

### Step 1: N′—((R)-2-(Benzyloxy)-2-(trifluoromethyl)hex-5-enoyl)-3-nitro-6-(((R)-pent-4-en-2-yl)oxy)-5-(trifluoromethyl)picolinohydrazide (101)

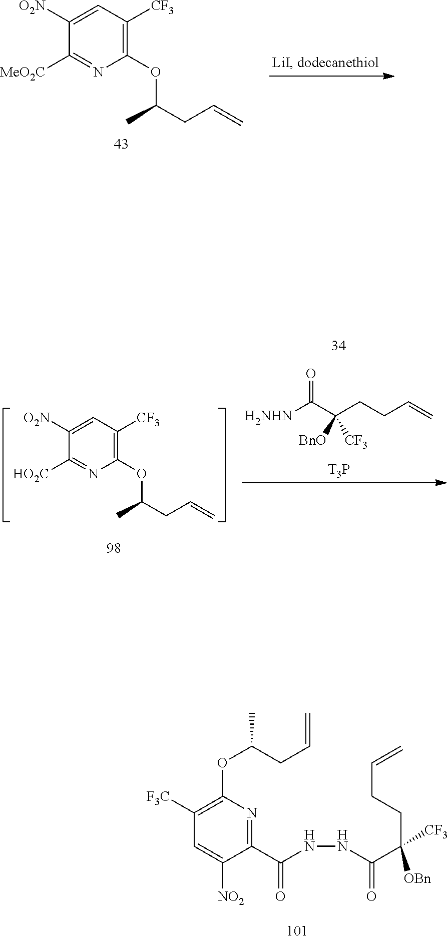

Methyl (R)-3-nitro-6-(pent-4-en-2-yloxy)-5-(trifluoromethyl)picolinate (15.1 g, 45.2 mmol) was charged to a 500 mL 3-neck round bottom flask. The flask was equipped with a stir paddle, an internal temperature probe, and a nitrogen inlet with a condenser. Ethyl acetate (106 mL) was charged and mixed at room temperature. The mixture was degassed while charging lithium iodide (6.73 g, 50.3 mmol) and 1-dodecanethiol (14.8 mL, 61.8 mmol). The reaction was set up under nitrogen with an air-cooled condenser and heated to a mild reflux (oil bath at 86° C. (Tint 75° C.)) for between 14 and 16 hours. The reaction was cooled with an ice bath <10° C., during which solids precipitated. (R)-2-(benzyloxy)-2-(trifluoromethyl)hex-5-enehydrazide (13.1 g, 43.3 mmol) was added using ethyl acetate (7.55 mL) to rinse the vessel to complete transfer of the substrate which is a viscous oil. Propylphosphonic anhydride (50:50 wt %) in ethyl acetate (32.3 mL, 54.2 mmol) was then added with an addition funnel dropwise keeping temperature <10° C. (Tmax 3.5° C.) which gave a clear yellow solution. N,N-diisopropylethylamine (10.4 mL, 59.7 mmol) was added with an addition funnel dropwise keeping temperature <10° C. (Tmax=4.8° C.), check pH (on wet paper about 3). The mixture was allowed to slowly warm to room temperature and after 1 hour the reaction was complete by HPLC. The reaction was cooled to <10° C. and diluted with 1.0 M aqueous hydrogen chloride (63.2 mL, 63.2 mmol, Tmax=5.6° C.). The phases were separated and the aqueous was back extracted with ethyl acetate (62 mL). The combined organic phases were washed two times each with water (52.8 mL). The organic solution was recharged to the reaction vessel and the volume was reduced by partial vacuum (190±10 torr) with oil bath at 65° C. The internal temperature was not measured but was approximately 45° C., the b.p. of ethyl acetate at 190±10 torr. The mixture was distilled to a targeted final volume between 40-45 mL. The thick yellow solution was held between 5° and 55° C. and heptane (317 mL) was added, maintaining temperature between 5° and 55° C. A slurry formed after about 210 mL were added. After the heptane addition was complete, the slurry was stirred at room temperature overnight. The slurry was filtered and the solids were washed six times with heptane (45 mL each). Isolated N′—((R)-2-(benzyloxy)-2-(trifluoromethyl)hex-5-enoyl)-3-nitro-6-(((R)-pent-4-en-2-yl)oxy)-5-(trifluoromethyl)picolinohydrazide (21.6 g) in a 79.1% yield. ¹H NMR (400 MHz, MeOH-d4) δ (ppm)=8.77-8.48 (m, 1H), 7.59-7.13 (m, 5H), 6.00-5.80 (m, 2H), 5.75-5.42 (m, 1H), 5.21-4.85 (m, 4H), 2.67-2.40 (m, 2H), 2.40-1.99 (m, 4H), 1.50-1.33 (m, 3H). ¹⁹F NMR (376 MHz, MeOH-d4) δ (ppm)=−74.63 (s, 3F), −65.77 (s, 3F). MS (ESI+) for C₂₆H₂₆F₆N₄O₆ m/z 605.2 (M+H)⁺ and MS (ESI-) for C₂₆H₂₆F₆N₄O₆ m/z 603.2 (M−H)⁻.

### Step 2: (3R,9R,Z)-9-(Benzyloxy)-3-methyl-1⁵-nitro-1³,9-bis(trifluoromethyl)-2-oxa-11,12-diaza-1(2,6)-pyridinacyclotridecaphan-5-ene-10,13-dione (102)

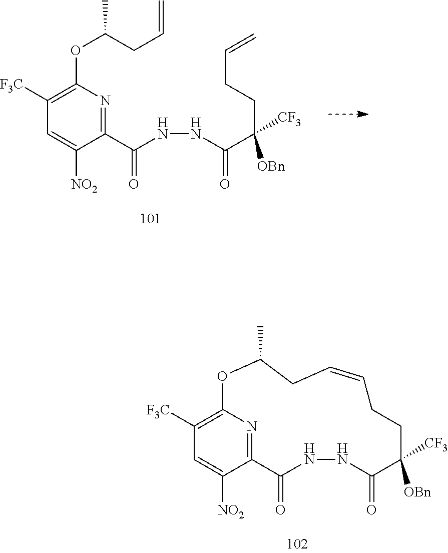 [SMILES: C=CCC[C@@](OCc1ccccc1)(C(=O)NNC(=O)c1nc(O[C@H](C)CC=C)c(C(F)(F)F)cc1[N+](=O)[O-])C(F)(F)F ; C[C@@H]1C/C=C\CC[C@](OCc2ccccc2)(C(F)(F)F)C(=O)NNC(=O)c2nc(c(C(F)(F)F)cc2[N+](=O)[O-])O1]

(3R,9R,Z)-9-(Benzyloxy)-3-methyl-1⁵-nitro-1³,9-bis(trifluoromethyl)-2-oxa-11,12-diaza-1(2,6)-pyridinacyclotridecaphan-5-ene-10,13-dione can be synthesized using a procedure analogous to Example 24, Step 8.

(3R,9R,Z)-9-(Benzyloxy)-3-methyl-1⁵-nitro-1³,9-bis(trifluoromethyl)-2-oxa-11,12-diaza-1(2,6)-pyridinacyclotridecaphan-5-ene-10,13-dione can also be synthesized using a procedure analogous to the following.

A solution of N′—((R)-2-(benzyloxy)-2-(trifluoromethyl)hex-5-enoyl)-3-nitro-6-(((R)-pent-4-en-2-yl)oxy)-5-(trifluoromethyl)picolinohydrazide (1 equiv.) in EtOAc (0.011 M, 150 Vols) is stirred at ambient temperature and sub-surface N₂ sparging is started. After 30 min of sparging, dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (Zhan Catalyst-1B, 0.1 equiv.) is added and the mixture is heated to 40° C. while continuing sub-surface N₂ sparging. After complete reaction by LC analysis, the mixture is cooled to ambient temperature. 2-Mercaptonicotinic acid (0.5 equiv.) followed by TEA (0.5 equiv.) are then charged to the reactor and the mixture is stirred overnight. Silica gel (6 equiv.) is added to mixture and stirred at least one hour. The mixture is filtered through a glass filter with a layer of silica gel and a thin layer of diatomaceous earth, washing the solids with EtOAc (40 Vols). The mixture is concerntrated to yield the crude product.

A slurry of combined crude material (1 equiv.) in MTBE (0.384 M, 4 Vols) is heated to 55° C. and stirred for 1 h. MTBE (1.921 M, 0.8 Vols) and heptane (0.48 M, 3.2 Vols) are added over 15 minutes. The mixture is stirred for an additional 30 minutes and then cooled to 10° C. over 2 h, followed by stirring overnight at 10° C.

The mixture is then filtered and the filter cake is first washed with MTBE (0.2 Vols) and then heptane (0.8 Vols). Air is pulled through the filter cake for 1 h. The filtered solvent is transferred to the rotary evaporator to dry at 50° C. for 4 h to afford (3R,9R,Z)-9-(benzyloxy)-3-methyl-1⁵-nitro-1³,9-bis(trifluoromethyl)-2-oxa-11,12-diaza-1(2,6)-pyridinacyclotridecaphan-5-ene-10,13-dione.

### Step 3: (4R,10R,Z)-10-(Benzyloxy)-4-methyl-2³-nitro-2⁵,10-bis(trifluoromethyl)-3-oxa-1(2,5)-oxadiazola-2(2,6)-pyridinacyclodecaphan-6-ene (100)

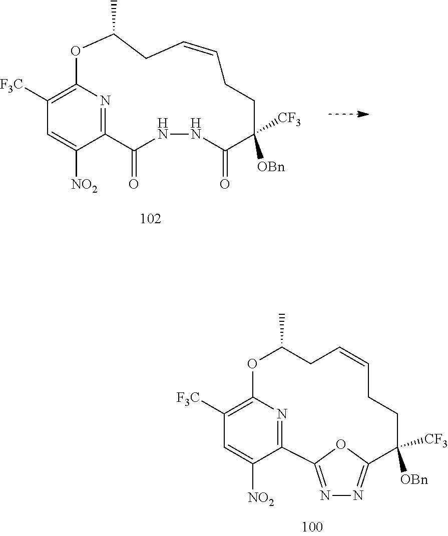 [SMILES: C[C@@H]1C/C=C\CC[C@](OCc2ccccc2)(C(F)(F)F)C(=O)NNC(=O)c2nc(c(C(F)(F)F)cc2[N+](=O)[O-])O1 ; C[C@@H]1C/C=C\CC[C@](OCc2ccccc2)(C(F)(F)F)C2=NN=C(O2)c2nc(c(C(F)(F)F)cc2[N+](=O)[O-])O1]

(4R,10R,Z)-10-(Benzyloxy)-4-methyl-2³-nitro-2⁵,10-bis(trifluoromethyl)-3-oxa-1(2,5)-oxadiazola-2(2,6)-pyridinacyclodecaphan-6-ene can be synthesized using a procedure analogous to Step 6 of Example 24.

(4R,10R,Z)-10-(Benzyloxy)-4-methyl-2³-nitro-2⁵,10-bis(trifluoromethyl)-3-oxa-1(2,5)-oxadiazola-2(2,6)-pyridinacyclodecaphan-6-ene can also be synthesized using a procedure analogous to the following.

A solution of (3R,9R,Z)-9-(benzyloxy)-3-methyl-1⁵-nitro-1³,9-bis(trifluoromethyl)-2-oxa-11,12-diaza-1(2,6)-pyridinacyclotridecaphan-5-ene-10,13-dione (1 equiv.) and 1,4-diazabicyclo[2.2.2]octane (DABCO; 1.5 equiv.) in DCM (8 VolEq) is stirred at RT when 25 w/v % 2-chloro-1,3-dimethylimidazolinium chloride (DMC; 1.1 equiv.) is added over 5 min while maintaining the reaction temperature between 15-30° C. Immediately after complete addition of DMC, the reaction mixtures is diluted with PhMe (2 VolEq) and concentrated to remove most of the DCM. The concentrate is diluted again with PhMe (8 VolEq). The suspension is heated at 100° C. The reaction temperature is maintained at 100-105° C. for 5-6 h until reaction completion. The suspension is cooled to RT and the solid (DABCO·HCl and N,N-dimethylimidazolidinone) is removed by filtration. The filter-cake is washed with MTBE (2 Vols) twice. The filtrate and washings are combined and washed sequentially, first with water (3 VolEq), then 0.5 M HCl (1 equiv.), and then water (3 VolEq). The mixture is then concentrated to afford crude (4R,10R,Z)-10-(benzyloxy)-4-methyl-2³-nitro-2⁵,10-bis(trifluoromethyl)-3-oxa-1(2,5)-oxadiazola-2(2,6)-pyridinacyclodecaphan-6-ene.

The crude (4R,10R,Z)-10-(benzyloxy)-4-methyl-2³-nitro-2⁵,10-bis(trifluoromethyl)-3-oxa-1(2,5)-oxadiazola-2(2,6)-pyridinacyclodecaphan-6-ene is dissolved in hot (75° C.) EtOH and cooled to RT over 1 h. The solid is collected by filtration and the filter-cake is washed with EtOH and then air-dried to afford (4R,10R,Z)-10-(benzyloxy)-4-methyl-2³-nitro-2⁵,10-bis(trifluoromethyl)-3-oxa-1(2,5)-oxadiazola-2(2,6)-pyridinacyclodecaphan-6-ene.

### Example 37: Bioactivity Assay Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101 and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

### Identification of Potentiator Compounds

The activity of the CFTR potentiator compounds on the CFTR-mediated I_{SC} was determined in Ussing chamber studies as described above. The F508del/MF-HBE cell cultures were incubated with the potentiator compounds at a range of concentrations in combination with 10 μM (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione for 18-24 hours at 37° C. and in the presence of 20% human serum. The concentration of potentiator compounds and (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione used during the 18-24 hours incubations was kept constant throughout the Ussing chamber measurement of the CFTR-mediated I_{SC} to ensure compounds were present throughout the entire experiment. The efficacy and potency of the putative F508del potentiators was compared to that of the known Vertex potentiator, ivacaftor (N-[2,4-bis(1,1-dimethylethyl)-5-hydroxyphenyl]-1,4-dihydro-4-oxoquinoline-3-carboxamide).

Table 56 presents CFTR modulating activity for Compound I using the assay described in this example (EC₅₀: +++ is <500 nM; ++ is 500 nM-1 μM; + is >1 μM; and ND is “not determined in this assay”).

TABLE 56
Assay Data for Compound I

| Molecule | EC₅₀ |
|---|---|
| Compound I | +++ |

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A crystalline Compound I 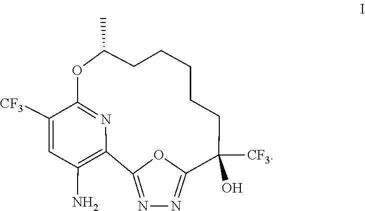 [SMILES: C[C@@H]1CCCCC[C@](O)(C(F)(F)F)C2=NN=C(O2)c2nc(c(C(F)(F)F)cc2N)O1]
wherein the crystalline Compound I is selected from substantially pure Compound I neat Form A, Compound I neat Form B, Compound I hemihydrate Form C, Compound I neat Form D, Compound I neat Form E, Compound I acetic acid solvate, Compound I heptane solvate Form B, Compound I heptane solvate Form C, Compound I octane solvate, Compound I cyclohexane solvate Form A, Compound I cyclohexane solvate Form B, Compound I cyclohexane solvate Form C, Compound I ethanol solvate, Compound I solvate/hydrate (dry), Compound I solvate/hydrate (wet), Compound I L-lysine cocrystal, Compound I L-arginine cocrystal, Compound I L-phenylalanine cocrystal, Compound I succinic acid cocrystal hydrate, Compound I succinic acid cocrystal, and Compound I methanol solvate/hydrate.

2.-3. (canceled)

4. The crystalline Compound I according to claim 1, wherein less than 5% of Compound I is in amorphous form.

5. (canceled)

6. Amorphous Compound I neat amorphous form.

7.-8. (canceled)

9. The amorphous Compound I according to claim 6, wherein less than 5% of Compound I is in crystalline form.

10.-13. (canceled)

14. A pharmaceutical composition comprising the Compound I according to any one of claims 1, 4, 6, or 9.

15. The pharmaceutical composition according to claim 14, further comprising one or more additional therapeutic agents.

16.-19. (canceled)

20. A method of treating cystic fibrosis comprising administering the Compound I according to any one of claims 1, 4, 6, or 9, or the pharmaceutical composition according to claim 14 or 15, to a subject in need thereof.

21. The method of claim 20, wherein the Compound I according to any one of claims 1, 4, 6, or 9 or the composition according to claim 14 or 15 is administered in combination with one or more additional therapeutic agents.

22.-24. (canceled)

25. A method of preparing Compound I: 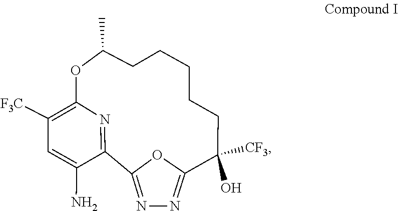 [SMILES: C[C@@H]1CCCCC[C@](O)(C(F)(F)F)C2=NN=C(O2)c2nc(c(C(F)(F)F)cc2N)O1]
or a stereoisomer of Compound I, or a deuterated derivative of Compound I or a stereoisomer thereof, or pharmaceutically acceptable salts of any of the foregoing, wherein the method comprises converting a compound of Formula (1): 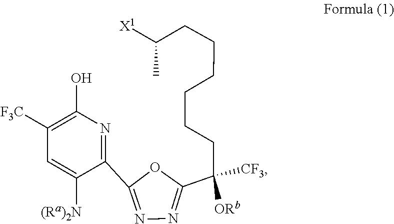
or a stereoisomer thereof, or a deuterated derivative of the compound of Formula (1) or its stereoisomer, or a salt of any of the foregoing, into Compound I, or a stereoisomer thereof or a deuterated derivative of Compound I or its stereoisomer, or pharmaceutically acceptable salt of any of the foregoing,
wherein:
X¹ is selected from OH, OTs, OMs, ONs, and OTf;
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
R^{b} is selected from benzyl (Bn), naphthylmethyl (Nap), biphenylmethyl, Ac, TFA, and Bz.

26. The method according to claim 25, wherein the method comprises converting a compound of Formula (2), 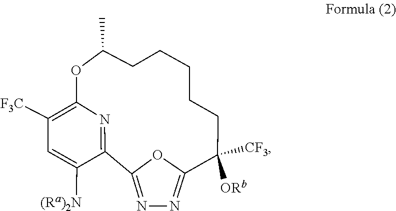
or a stereoisomer thereof, or a deuterated derivative of the compound of Formula (2) or its stereoisomer, or a salt of any of the foregoing, into Compound I, or a stereoisomer thereof, or a deuterated derivative of Compound I or its stereoisomer, or a pharmaceutically acceptable salt of any of the foregoing,
wherein:
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
R^{b} is selected from benzyl (Bn), naphthylmethyl (Nap), biphenylmethyl, Ac, TFA, and Bz.

27. The method according to claim 25, wherein the method comprises converting a compound of Formula (3): 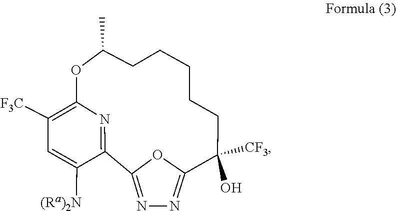
or a stereoisomer thereof, or a deuterated derivative of the compound of Formula (3) or its stereoisomer, or a salt of any of the foregoing, into Compound I, or a stereoisomer thereof, or a deuterated derivative of Compound I or its stereoisomer, or a pharmaceutically acceptable salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).

28.-36. (canceled)

37. A method of preparing Compound I: 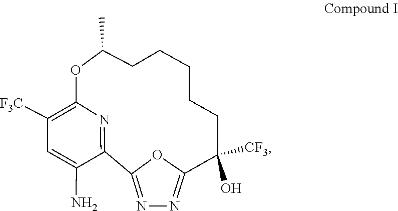 [SMILES: C[C@@H]1CCCCC[C@](O)(C(F)(F)F)C2=NN=C(O2)c2nc(c(C(F)(F)F)cc2N)O1]
or a stereoisomer thereof, or a deuterated derivative of the compound of Formula (1) or its stereoisomer, or a salt of any of the foregoing, comprising converting a compound of Formula (18): 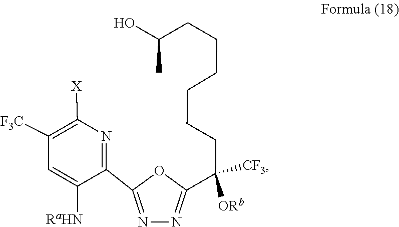
or a stereoisomer thereof, or a deuterated derivative of the compound of Formula (18) or its stereoisomer, or a salt of any of the foregoing, into Compound I, or a stereoisomer thereof, or a deuterated derivative of the compound of Formula (1) or its stereoisomer, or a salt of any of the foregoing,
wherein:
X is selected from Cl, Br, I, —OSO₂R, and —SR,
R is selected from Me, —CF₃, Ph, and 4-MePh;
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz;

38. The method according to claim 37, wherein the method comprises converting a compound of Formula (2a): 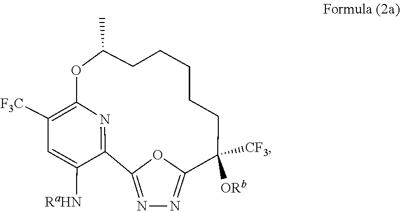
or a stereoisomer thereof, or a deuterated derivative of the compound of Formula (2a) or its stereoisomer, or a salt of any of the foregoing, into Compound I, or a stereoisomer thereof, or a deuterated derivative of the compound of Formula (1) or its stereoisomer, or a salt of any of the foregoing,
wherein:
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz;

39. The method according to claim 38, wherein the method comprises converting a compound of Formula (3a): 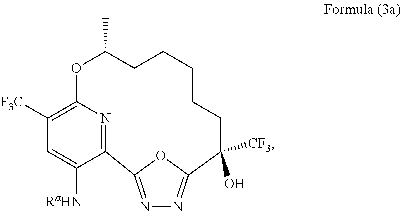
or a stereoisomer thereof, or a deuterated derivative of the compound of Formula (3a) or its stereoisomer, or a salt of any of the foregoing, into Compound I, or a stereoisomer thereof, or a deuterated derivative of the compound of Formula (1) or its stereoisomer, or a salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).

40.-46. (canceled)

47. A method of preparing Compound I: 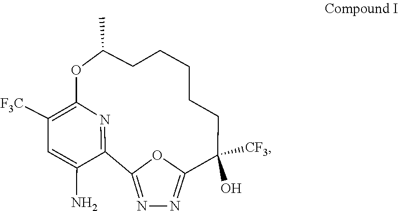 [SMILES: C[C@@H]1CCCCC[C@](O)(C(F)(F)F)C2=NN=C(O2)c2nc(c(C(F)(F)F)cc2N)O1]
or a stereoisomer thereof, or a deuterated derivative of Compound I or its stereoisomer, or a salt of any of the foregoing, comprising converting a compound of Formula (32): 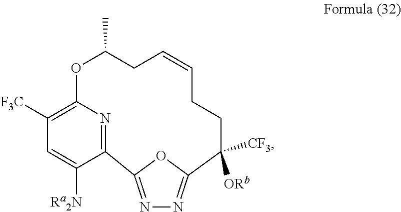
or a stereoisomer thereof, or a deuterated derivative of the compound of Formula (32) or its stereoisomer, or a salt of any of the foregoing, into Compound I, or a stereoisomer thereof, or a deuterated derivative of Compound I or its stereoisomer, or a salt of any of the foregoing,
wherein:
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns) or N(R^{a})₂ is NO₂; and
R^{b} is selected from benzyl (Bn), naphthylmethyl (Nap), biphenylmethyl, Ac, TFA, and Bz.

48. The method according to claim 47, wherein the method comprises converting a compound of Formula (33): 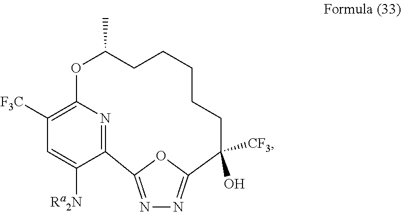
or a stereoisomer thereof, or a deuterated derivative of the compound of Formula (33) or its stereoisomer, or a salt of any of the foregoing, into Compound I, or a stereoisomer thereof, or a deuterated derivative of Compound I or its stereoisomer, or a salt of any of the foregoing,
wherein:
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

49. The method according to claim 48, wherein the compound of Formula (33): 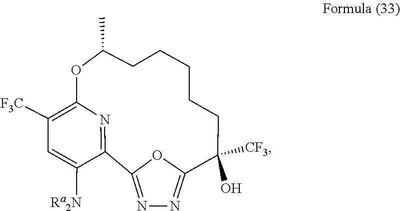
or a stereoisomer thereof, or a deuterated derivative of the compound of Formula (33) or its stereoisomer, or a salt of any of the foregoing, is prepared by converting a compound of Formula (32): 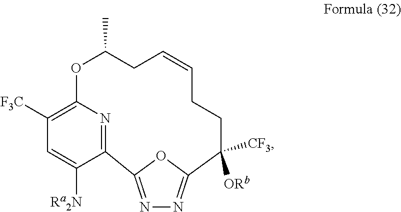
or a stereoisomer thereof, or a deuterated derivative of the compound of Formula (32) or its stereoisomer, or a salt of any of the foregoing, into the compound of Formula (33), or a stereoisomer thereof, or a deuterated derivative of the compound of Formula (33) or its stereoisomer, or a salt of any of the foregoing,
wherein:
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

50. The method according to claim 49, wherein the compound of Formula (32): 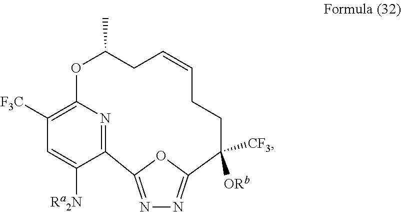
or a stereoisomer thereof, or a deuterated derivative of the compound of Formula (32) or its stereoisomer, or a salt of any of the foregoing, is prepared by converting a compound of Formula (34): 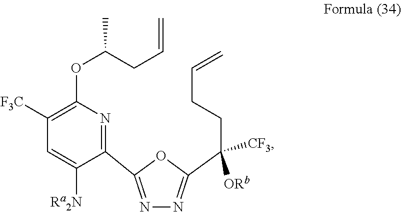
or a stereoisomer thereof, or a deuterated derivative of the compound of Formula (34) or its stereoisomer, into the compound of Formula (32), or a stereoisomer thereof, or a deuterated derivative of the compound of Formula (32) or its stereoisomer, or a salt of any of the foregoing,
wherein:
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or N(R^{a})₂ is NO₂; and
R^{b} is selected from benzyl (Bn), naphthylmethyl (Nap), biphenylmethyl, Ac, TFA, and Bz.

51. The method according to claim 50, wherein the compound of Formula (32): 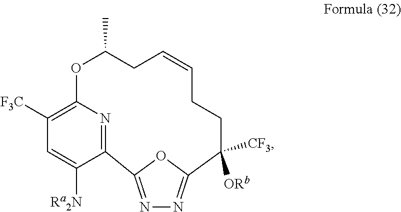
or a stereoisomer thereof, or a deuterated derivative of the compound of Formula (32) or its stereoisomer, or a salt of any of the foregoing, is prepared by converting a compound of Formula (36a): 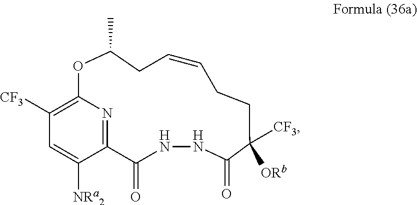
or a stereoisomer thereof, or a deuterated derivative of the compound of Formula (36a) or its stereoisomer, into the compound of Formula (32), or a stereoisomer thereof, or a deuterated derivative of the compound of Formula (32) or its stereoisomer, or a salt of any of the foregoing,
wherein:
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or NR^{a}₂ is NO₂; and
R^{b} is selected from benzyl (Bn), naphthylmethyl (Nap), biphenylmethyl, Ac, TFA, and Bz.

52. The method according to claim 51, wherein the compound of Formula (36a): 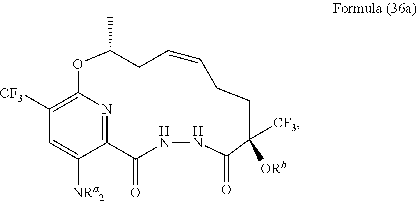
or a stereoisomer thereof, or a deuterated derivative of the compound of Formula (36a) or its stereoisomer, or a salt of any of the foregoing, is prepared by converting a compound of Formula (36b): 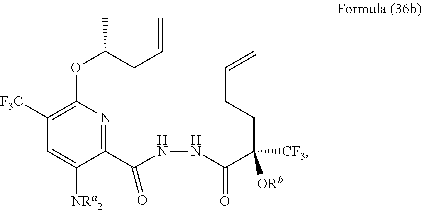
or a stereoisomer thereof, or a deuterated derivative of the compound of Formula (36b) or its stereoisomer, into the compound of Formula (36a), or a stereoisomer thereof, or a deuterated derivative of the compound of Formula (36a) or its stereoisomer, or a salt of any of the foregoing,
wherein:
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns), or NR^{a}₂ is NO₂; and
R^{b} is selected from benzyl (Bn), naphthylmethyl (Nap), biphenylmethyl, Ac, TFA, and Bz.

53. The method according to claim 50, wherein the compound of Formula (34): 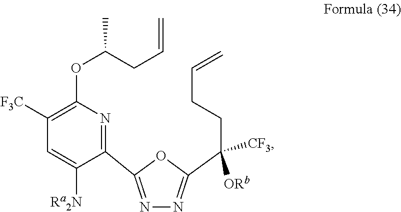
or a stereoisomer thereof, or a deuterated derivative of the compound of Formula (34) or its stereoisomer, is prepared by converting a compound of Formula (35): 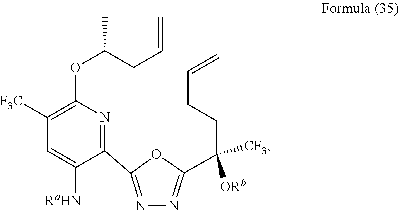
or a stereoisomer thereof, or a deuterated derivative of the compound of Formula (35) or its stereoisomer, or a salt of any of the foregoing, into the compound of Formula (34), or a stereoisomer thereof, or a deuterated derivative of the compound of Formula (34) or its stereoisomer,
wherein:
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

54. The method according to claim 53, wherein the compound of Formula (35): 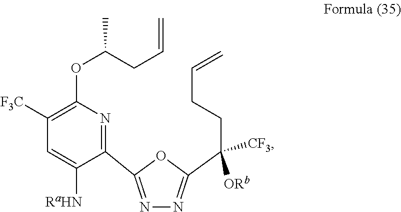
or a stereoisomer thereof, or a deuterated derivative of the compound of Formula (35) or its stereoisomer, or a salt of any of the foregoing, is prepared by converting a compound of Formula (36): 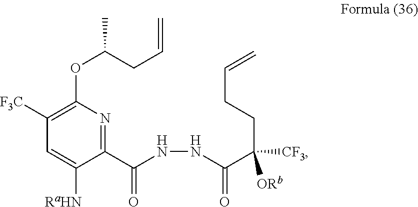
or a stereoisomer thereof, or a deuterated derivative of the compound of Formula (36) or its stereoisomer, or a salt of any of the foregoing, into the compound of Formula (35), or a stereoisomer thereof, or a deuterated derivative of the compound of Formula (35) or its stereoisomer, or a salt of any of the foregoing,
wherein:
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

55. The method according to claim 54, wherein the compound of Formula (36): 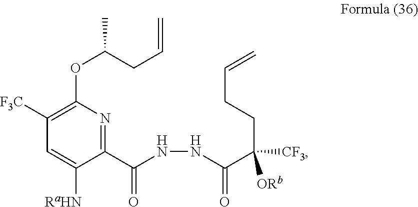
or a stereoisomer thereof, or a deuterated derivative of the compound of Formula (36) or its stereoisomer, or a salt of any of the foregoing, is prepared by combining a compound of Formula (37): 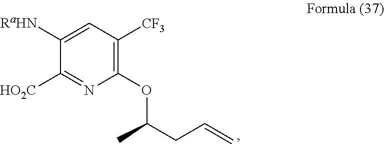
or a deuterated derivative or salt thereof, with a compound of Formula (38): 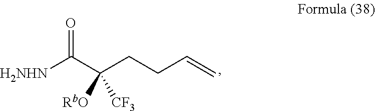
or a stereoisomer thereof, or a deuterated derivative of the compound of Formula (38) or its stereoisomer, or a salt of any of the foregoing, to produce the compound of Formula (36), or a stereoisomer thereof, or a deuterated derivative of the compound of Formula (36) or its stereoisomer, or a salt of any of the foregoing,
wherein:
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

56. (canceled)

57. The method according to claim 47 or claim 49, wherein the compound of Formula (32): 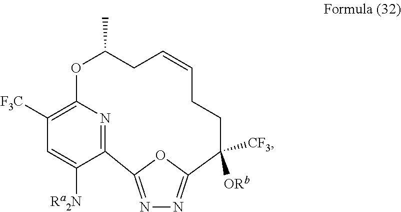
or a stereoisomer thereof, or a deuterated derivative of the compound of Formula (32) or its stereoisomer, or a salt of any of the foregoing, is prepared by converting a compound of Formula (42): 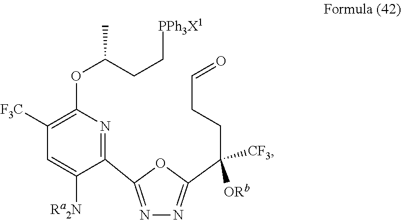
or a stereoisomer thereof, or a deuterated derivative of the compound of Formula (42) or its stereoisomer, or a salt of any of the foregoing, into the compound of Formula (32), or a stereoisomer thereof, or a deuterated derivative of the compound of Formula (32) or its stereoisomer, or a salt of any of the foregoing,
wherein:
X¹ is selected from Cl, Br, and I;
each R^{a} is independently selected from H, tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

58.-63. (canceled)

64. A method of preparing Compound I: 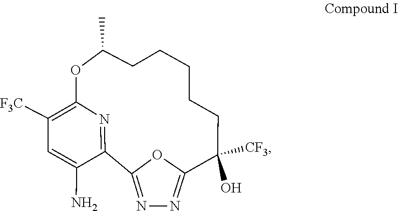 [SMILES: C[C@@H]1CCCCC[C@](O)(C(F)(F)F)C2=NN=C(O2)c2nc(c(C(F)(F)F)cc2N)O1]
or a stereoisomer thereof, or a deuterated derivative of the Compound I or its stereoisomer, or a salt of any of the foregoing, comprising converting a compound of Formula (51): 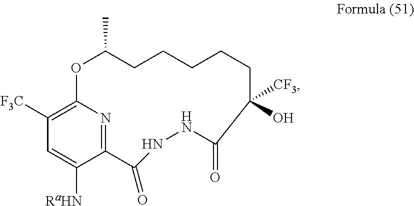
or a stereoisomer thereof, or a deuterated derivative of the compound of Formula (51) or its stereoisomer, or a salt of any of the foregoing, into Compound I, or a stereoisomer thereof, or a deuterated derivative of the Compound I or its stereoisomer, or a salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).

65. The method according to claim 64, wherein the method comprises converting a compound of Formula (52): 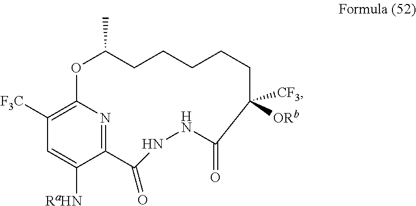
or a stereoisomer thereof, or a deuterated derivative of the compound of Formula (52) or its stereoisomer, or a salt of any of the foregoing, into Compound I, or a stereoisomer thereof, or a deuterated derivative of the Compound I or its stereoisomer, or a salt of any of the foregoing, wherein:
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

66. (canceled)

67. The method according to claim 64, wherein the method comprises converting a compound of Formula (3a): 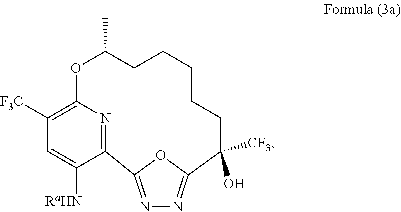
or a stereoisomer thereof, or a deuterated derivative of the compound of Formula (3a) or its stereoisomer, or a salt of any of the foregoing, into Compound I, or a stereoisomer thereof, or a deuterated derivative of the Compound I or its stereoisomer, or a salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).

68. (canceled)

69. The method according to claim 64, wherein the method comprises converting a compound of Formula (2a): 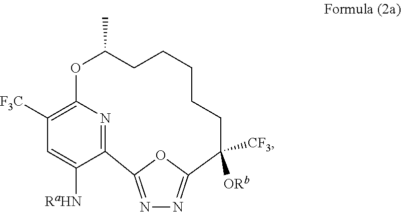
or a stereoisomer thereof, or a deuterated derivative of the compound of Formula (2a) or its stereoisomer, or a salt of any of the foregoing, into Compound I, or a stereoisomer thereof, or a deuterated derivative of the Compound I or its stereoisomer, or a salt of any of the foregoing,
wherein:
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

70. The method according to claim 69, wherein the compound of Formula (2a): 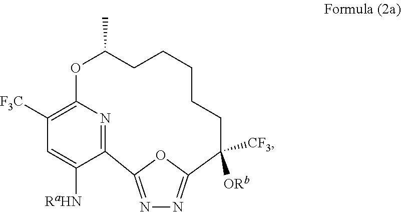
or a stereoisomer thereof, or a deuterated derivative of the compound of Formula (2a) or its stereoisomer, or a salt of any of the foregoing, is prepared by converting a compound of Formula (51): 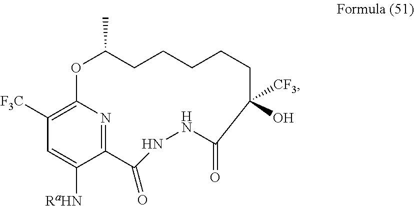
or a stereoisomer thereof, or a deuterated derivative of the compound of Formula (51) or its stereoisomer, or a salt of any of the foregoing, into the compound of Formula (2a), or a stereoisomer thereof, or a deuterated derivative of the compound of Formula (2a) or its stereoisomer, or a salt of any of the foregoing,
wherein:
R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns); and
R^{b} is selected from benzyl (Bn), naphthylmethyl (Nap), biphenylmethyl, Ac, TFA, and Bz.

71. The method according to claim 70, wherein the compound of Formula (51): 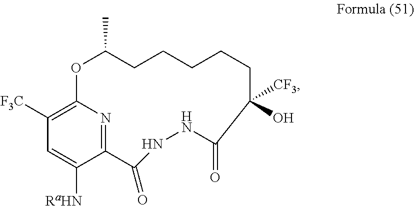
or a stereoisomer thereof, or a deuterated derivative of the compound of Formula (51) or its stereoisomer, or a salt of any of the foregoing, is prepared by converting a compound of Formula (53): 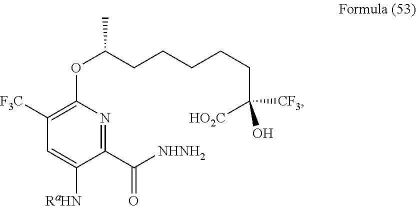
or a stereoisomer thereof, or a deuterated derivative of the compound of Formula (53) or its stereoisomer, or a salt of any of the foregoing, into the compound of Formula (51), or a stereoisomer thereof, or a deuterated derivative of the compound of Formula (51) or its stereoisomer, or a salt of any of the foregoing, wherein R^{a} is selected from tert-butyloxycarbonyl (Boc), fluorenylmethoxycarbonyl (Fmoc), phthalimido (Phth), acetyl (Ac), trifluoroacetyl (TFA), pivaloyl (Piv), benzoyl (Bz), carbobenzyloxy (Cbz), methanesulfonyl (Ms), and nitrobenzenesulfonyl (Ns).

72.-75. (canceled)

76. A compounds selected from: 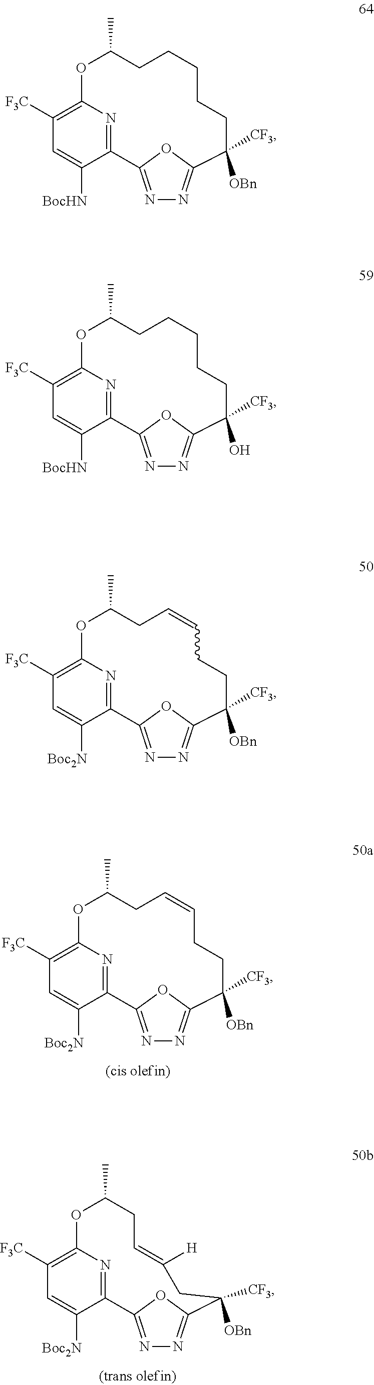 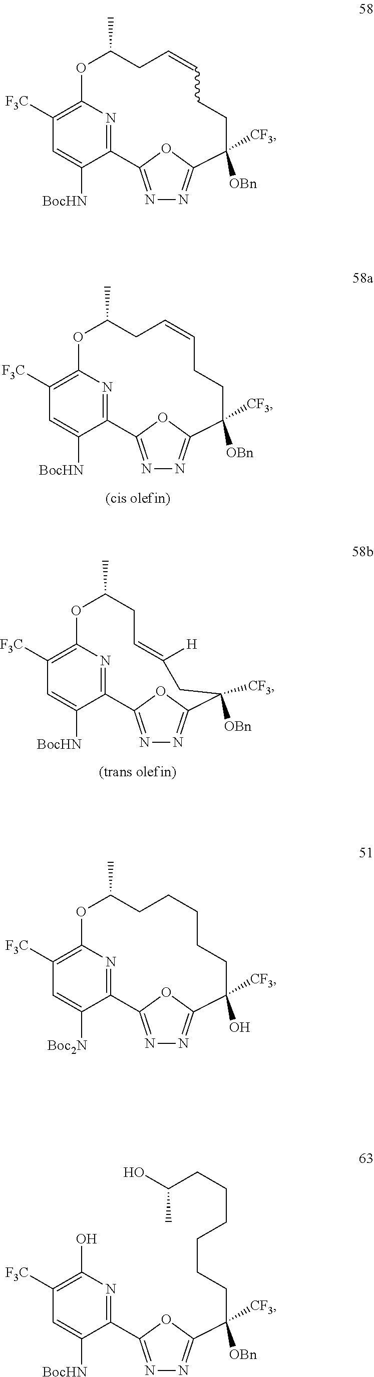 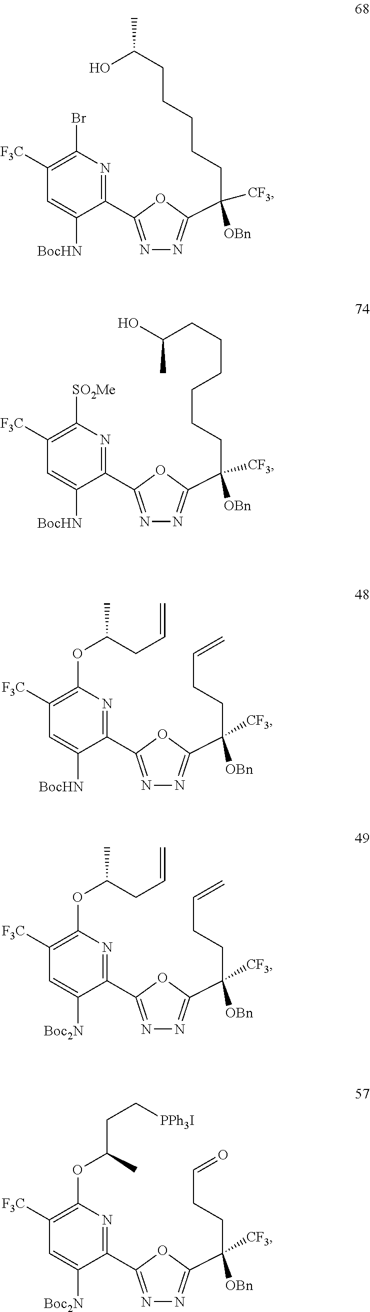 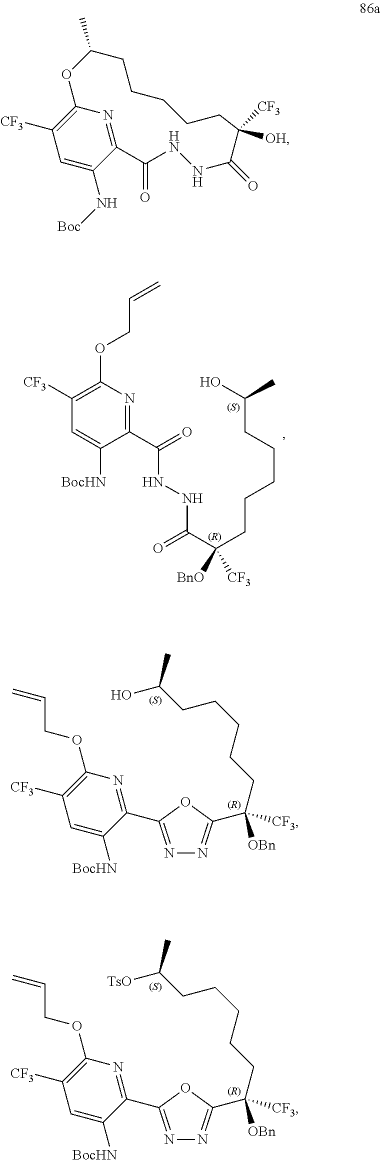
stereoisomers is thereof, deuterated derivatives of any of the foregoing, and salts of any of the foregoing.

77.-78. (canceled)
